(19) United States
(12) Patent Application Publication
(10) Pub. No.: US 2024/0382483 A1
(43) Pub. Date: Nov. 21, 2024

(54) HETEROCYCLIC EGFR INHIBITORS FOR USE IN THE TREATMENT OF CANCER

(71) Applicant: Blueprint Medicines Corporation, Cambridge, MA (US)

(72) Inventors: Natasja Brooijmans, Cambridge, MA (US); John Emmerson Campbell, Cambridge, MA (US); Christopher De Savi, Cambridge, MA (US); Thomas A. Dineen, Cambridge, MA (US); Meredith Suzanne Eno, Cambridge, MA (US); Joseph L. Kim, Cambridge, MA (US); Aysegul Ozen, Cambridge, MA (US); Emanuele Perola, Cambridge, MA (US); Brett D. Williams, Cambridge, MA (US); Douglas Wilson, Cambridge, MA (US); Kevin J. Wilson, Cambridge, MA (US)

(21) Appl. No.: 18/572,382

(22) PCT Filed: Jun. 21, 2022
(86) PCT No.: PCT/US2022/034213

§ 371 Date: Dec. 20, 2023

Related U.S. Application Data
(60) Provisional application No. 63/213,363, filed on Jun. 22, 2021.

(51) Int. Cl. A61K 31/506; C07D 401/14; C07D 413/14; C07D 471/04; C07D 495/10; C07D 519/00; C07F 9/6561

(52) U.S. Cl. CPC A61K 31/506; C07D 401/14; C07D 413/14; C07D 471/04; C07D 495/10; C07D 519/00; C07F 9/6561

(57) ABSTRACT

The present disclosure provides a compound represented by structural formula (I) or a pharmaceutically acceptable salt thereof useful for treating a cancer.

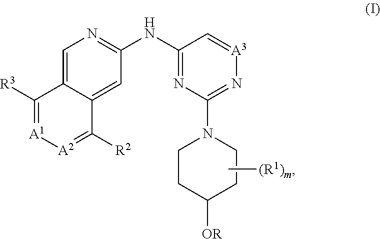

### CROSS-REFERENCE TO RELATED APPLICATIONS

This application claims priority to U.S. Provisional Application No. 63/213,363, filed on Jun. 22, 2021. The entire contents of the aforementioned application are incorporated herein by reference.

### TECHNICAL FIELD

This disclosure relates to compounds and compositions useful for treating disorders related to certain mutant forms of EGFR.

### BACKGROUND

EGFR (Epidermal Growth Factor Receptor) is a member of the erbB receptor family, which includes transmembrane protein tyrosine kinase receptors. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors, EGFR gene amplification and alterations such as mutations, deletions and the like, can promote malignant transformation of cells and play an important role in tumor cell proliferation, invasion, metastasis and angiogenesis. For example, alterations such as mutations and deletions in the EGFR gene are found in non-small lung cancer (NSCLC) tumors. The two most frequent EGFR alternations found in NSCLC tumors are short in-frame deletions in exon 19 (de119) and L858R, a single missense mutation in exon 21 (Cancer Discovery 2016 6(6) 601). These two alterations cause ligand-independent EGFR activation and are referred to as primary or activating mutations in EGFR mutant NSCLC (EGFR M+). Clinical experience shows an objective response rate (ORR) of approximately 60-85% in EGFR M+ NSCLC patients treated first line (1L) with EGFR tyrosine kinase inhibitors (TKIs) erlotinib, gefitinib, afatinib and osimertinib (Lancet Oncol. 2010 Vol. 11, 121; Lancet Oncol. 2016 Vol. 17, 577; N. Engl. J. Med. 2017 Nov. 18 Doi:10.1056/NEJMoa1713137; Lancet Oncol. 2011 Vol. 12, 735), thus demonstrating that EGFR mutant NSCLC tumors depend on oncogenic EGFR activity for survival and proliferation and establishing del19 and L858R mutated EGFR as oncogenic drivers of disease and thus, validating drug targets and biomarkers for the treatment of NSCLC.

However, after an average of 10-12 months of treatment with first generation (erlotinib and gefitinib) and second generation (afatinib) EGFR TKIs, resistance to these small molecule inhibitors has been observed in almost all NSCLC patients (Lancet Oncol. 2010 February; 11(2):121-8; Lancet Oncol. 2016 May; 17(5):577-89; Lancet Oncol. 2011 August; 12(8):735-42). The most prominent resistance mechanism to first and second generation EGFR TKIs is due to the secondary mutation in EGFR of T790M, occurs in 50% to 70% of patients progressing on 1st and 2nd generation EGFR inhibitors. (Blakely, Cancer Discov; 2(10); 872-5, 2012; Kobayashi, Cancer Res., 65:(16), 2005). This secondary mutation reduces the affinity of the drug with the target, thereby producing drug resistance, and resulting in tumor recurrence or disease progression.

In view of the prevalence of this mutation in drug resistance produced in therapy targeting EGFR of lung cancer, a number of companies have attempted to develop new small molecule EGFR inhibitors for treating these patients with drug-resistant lung cancer by inhibiting the resistant mutant EGFR-T790M. For example, osimertinib (Tagrisso®), a third generation EGFR TKI, has been developed to treat NSCLC patients if the cancer cells are positive for the primary EGFR mutations del19 or L858R with or without the T790M mutation in the gene coding for EGFR.

Although the third generation EGFR TKI, osimertinib, has shown efficacy on NSCLC patients, unfortunately, resistance mediated by an exon 20 C797 mutation in EGFR usually develops within approximately 10 months (European Journal of Medicinal Chemistry 2017 Vol. 142: 32-47) and accounts for the majority of osimertinib resistance cases (Cancer Letters 2016 Vol. 385: 51-54). The EGFR del19/L858R T790M C797S cis mutant kinase variant typically emerges in second line (2L) patients following treatment with osimertinib and is often referred to as “triple mutant” EGFR and it can no longer be inhibited by first, second, or third generation EGFR inhibitors.

No approved EGFR TKI can inhibit the triple mutant variant. Therefore, there is a need to develop new EGFR inhibitors, which can inhibit with high selectivity EGFR mutants with the triple mutant, del19/L858R T790M C797S, while at the same time have no or low activity to wild-type EGFR. In addition to treating a mutant form of EGFR for which there is no current therapy, such selective EGFR inhibitors are likely to be more suitable as therapeutic agents, particularly for the treatment of cancer, due to reduction of toxicologies (diarrhea, skin rash) associated with wild-type EGFR inhibition.

### SUMMARY

The applicant has discovered novel compounds which are effective inhibitors of certain mutant forms of EGFR (see Synthetic Examples 1-219). In particular, it has been demonstrated that the compounds of the present disclosure effectively inhibit certain mutant forms of EGFR. Compounds of the disclosure (also referred to herein as the “disclosed compounds”) or pharmaceutically acceptable salts thereof effectively inhibit EGFR with one or more alterations, including L858R and/or exon 19 deletion mutation, T790M mutation, and/or C797S mutation. Compounds of the disclosure or pharmaceutically acceptable salts thereof effectively inhibit EGFR with L858R and/or exon 19 deletion mutation, T790M mutation, and C797S mutation (hereinafter “EGFR with LRTMCS mutations” or “triple mutant EGFR”) (see Biological Example 1) and can be used treat various cancers, for example, lung cancer (see Biological Example 2). Importantly, the disclosed compounds are selective EGFR inhibitors, i.e., the disclosed compounds have no or low activity against wild-type EGFR and the kinome. Advantages associated with such selectivity may include facilitating efficacious dosing and reducing EGFR-mediated on-target toxicities. Some of the disclosed compounds exhibit good penetration of the brain and blood brain barrier (e.g., a PGP efflux ratio of less than 5). As such, the compounds of the disclosure or pharmaceutically acceptable salts thereof are expected to be effective for the treatment of metastatic cancer, including brain metastesis, including leptomeningeal disease and other systemic metastesis. Some of the disclosed compounds also have the advantage of having high microsomal stability. Compounds of the disclosure also may have favorable toxicity profiles related to other non-kinase targets.

In one aspect, the present disclosure provides a compound represented by the following structural Formula (I):

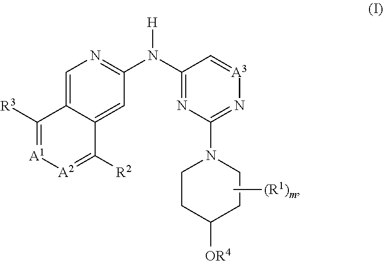

or a pharmaceutically acceptable salt thereof, wherein:
   each A¹, A², and A³ is independently N or CR; wherein each R is independently H, halogen, or CH₃;
   each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ or in the group represented by R¹ is optionally substituted with 1 to 3 groups selected from deuterium, halogen, OH, NR^{a}R^{b}, C₁-C₂ alkyl, and C₁-C₂ alkoxy; or two R¹, attached to the same carbon atom, together with the carbon atom to which both are attached form a C₃-C₄cycloalkyl optionally substituted with 1 to 3 groups selected from deuterium, halogen, OH, NR^{a}R^{b}, C₁-C₂ alkyl, and C₁-C₂ alkoxy; and/or
   m is 0, 1, 2, 3, 4, 5, or 6;
   R² is H, halogen, C₁-C₆ alkyl, C₁-C₆ alkoxy, C₃-C₆ cycloalkyl, 4 to 8 membered heterocyclyl, or 5 to 12-membered heteroaryl, wherein the alkyl, alkoxy, cycloalkyl, or heterocyclyl represented by R² are optionally substituted with 1 to 3 groups selected from R^{2a};
   Each R^{2a} is independently selected from halogen, CN, OH, C(O)NR^{a}R^{b}, C₁-C₄ alkyl, C₁-C₄ alkoxy, and 4 to 8 membered heterocyclyl, wherein the alkoxy represented by R^{2a} is optionally substituted with 4 to 8 membered heterocyclyl, and the heterocyclyl represented by R^{2a} or in the group represented by R^{2a} is optionally substituted with C₁-C₄alkyl;
   R³ is C₁-C₄ alkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is C₃-C₆cycloalkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is 5 to 12 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O;
R³ is a 4 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O; or
R³ is 5 to 12-membered heteroaryl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O;
R⁴ is H, or C₁-C₄alkyl optionally substituted with 1 to 3 groups selected from deuterium, OR^{a}, and NR^{a}R^{b}, or R⁴, together with R¹ attached to the same carbon atom, together with their intervening atoms form a 3 to 5 membered heterocyclyl;
each R^{a} and R^{b} is independently H or C₁-C₄ alkyl optionally substituted with 1 to 3 groups selected from deuterium, halogen, OH and NH₂; and
each R^{c} is independently C₁-C₄ alkyl optionally substituted with 1 to 3 halogen.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (I)) or a pharmaceutically acceptable salt thereof or a pharmaceutical composition of the disclosure. In one embodiment, the cancer is non-small cell lung cancer. In another embodiment, the subject cancer has metastasized to the brain. In another embodiment, the subject has brain metastasis from non-small cell lung cancer.

In one embodiment, the cancer to be treated has epidermal growth factor receptor (EGFR) L858R mutation and/or exon 19 deletion mutation and T790M mutation. In another embodiment, the cancer to be treated may further has epidermal growth factor receptor (EGFR) L858R mutation and/or exon 19 deletion mutation and the T790M mutation and the C797S mutation. In another embodiment, the cancer to be treated in either of the foregoing embodiments is lung cancer, e.g., non-small cell lung cancer. In a specific embodiment, the cancer is non-small cell lung cancer with brain metastasis.

The treatment method disclosed herein further comprises administering to the subject an effective amount of afatinib, osimertinib, erlotinib, or gefitinib.

The present disclosure also provides a method of inhibiting epidermal growth factor receptor (EGFR) in a subject in need thereof, comprising administering to the subject an effective amount of a compound of the disclosure (e.g., a compound of Formula (I)) or a pharmaceutically acceptable salt thereof or a pharmaceutical composition of the disclosure.

The present disclosure also provides the use of an effective amount of a compound of the disclosure (e.g., a compound of Formula (I)), or a pharmaceutically acceptable salt thereof, or a pharmaceutical composition of the disclosure, for the preparation of a medicament for the treatment of cancers.

In another aspect, provided herein a compound of Formula (I), or a pharmaceutically acceptable salt thereof, or a pharmaceutical composition of the disclosure for use in treating cancers.

### DETAILED DESCRIPTION

### Definitions

The term “halo” as used herein means halogen and includes chloro, fluoro, bromo and iodo.

The term “alkyl” used alone or as part of a larger moiety, such as “alkoxy” and the like, means saturated aliphatic straight-chain or branched monovalent hydrocarbon radical. Unless otherwise specified, an alkyl group typically has 1-4 carbon atoms, i.e. (C₁-C₄)alkyl. As used herein, a “(C₁-C₄)alkyl” group means a radical having from 1 to 4 carbon atoms in a linear or branched arrangement. Examples include methyl, ethyl, n-propyl, iso-propyl, n-butyl, iso-butyl, sec-butyl, and the like.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “aryl” refers to a monovalent radical of an aromatic hydrocarbon ring system. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like.

The term “cycloalkyl” refers to a monocyclic saturated hydrocarbon ring system. Unless otherwise specified, cycloalkyl has from 3-6 carbon atoms. For example, a C₃-C₆ cycloalkyl includes cyclopropyl, cyclobutyl, cyclopentyl and cyclohexyl.

“Heteroaryl” refers to a monovalent radical of a 5- to 12-membered (or 5- to 10-membered) heteroaromatic ring system. A heteroaryl has ring carbon atoms and 1 to 4 ring heteroatoms, independently selected from O, N, and S. Representative heteroaryl groups include ring systems (e.g., monocyclic, bicyclic, or polycyclic) where: (i) each ring comprises a heteroatom and is aromatic, e.g., imidazolyl, oxazolyl, isoxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3-(4H)-one, 5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl.

The term “heterocyclyl” or “heterocyclic” refers to a radical of a 4- to 12- (or 4 to 10)-membered saturated or partially saturated ring system (“4-12 membered heterocyclyl” or (“4-10 membered heterocyclyl”) having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone. In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or tricyclic system (“tricyclic heterocyclyl”); and bicyclic and polycyclic ring systems include fused, bridged, or spiro ring systems). Exemplary monocyclic heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system. Substituents (e.g., R¹) may be present on one or more rings in the polycyclic ring system.

Representative heterocyclyls include ring systems in which: (i) every ring is non-aromatic and at least one ring comprises a heteroatom, e.g., tetrahydrofuranyl, tetrahydropyranyl, oxetanyl, azetidinyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, quinuclidinyl, and (3aR,6aS)-hexahydro-1□²-furo[3,4-b]pyrrole; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom e.g., 6,7-dihydro-5H-pyrrolo[1,2-c]imidazole.

In some embodiments, a heterocyclyl group is a 8-12 membered bicyclic heterocyclyl, e.g., wherein a saturated or partially saturated heterocyclyl is fused to an aromatic or heteroaromatic ring. The term “heterocyclyl” can also include 8-12 membered bicyclic heterocyclyls, wherein a saturated or partially saturated cycloalkyl is fused to an aromatic or heteroaromatic ring. The point of attachment of the heterocyclyl to the rest of the molecule can be through the saturated or partially saturated heterocyclyl or cycloalkyl, or through the aromatic or heteroaromatic ring.

In some embodiments, a bridged bicyclic system has at two non-aromatic rings containing from 7-12 ring atoms (heterocyclyl or cycloalkyl) and which share three or more atoms, with the two bridgehead atoms separated by a bridge containing at least one atom. “Bridged heterocyclyl” includes bicyclic or polycyclic hydrocarbon or aza-bridged hydrocarbon groups; examples include 2-azabicyclo[2.2.1]heptanyl, 3-azabicyclo[3.2.1]octanyl, 6-oxa-2-azabicyclo[3.2.1]octanyl, 6-oxa-3-azabicyclo[3.2.1]octanyl, and 8-oxa-3-azabicyclo[3.2.1]octanyl.

In some embodiments, a fused bicyclic system has two non-aromatic rings (heterocyclyl or cycloalkyl) containing from 7-12 ring atoms and which share two adjacent ring atoms. Examples of fused bicyclic systems include hexahydro-1H-furo[3,4-b]pyrrolyl, hexahydro-1H-furo[3,4-c]pyrrolyl, 6,7-dihydro-5H-pyrrolo[1,2-c]imidazole, (3aR,6aS)-hexahydro-1□²-furo[3,4-b]pyrrole.

In some embodiments, a spiro bicyclic system has two non-aromatic rings containing (heterocyclyl or cycloalkyl) from 7-12 ring atoms and which share one ring atom. Examples of spiro bicyclic systems include 1-oxa-7-azaspiro[3.5]nonan-7-yl, 1,4-dioxa-8-azaspiro[4.5]decan-8-yl, and 1,4-dioxa-9-azaspiro[5.5]undecan-9-yl.

### Compounds of the Present Disclosure

Disclosed herein are embodiments of compounds having a general structure of Formula (I). These compounds are selective inhibitors of LRTM and LRTMCS EGFR. In contrast to other EGFR inhibitors such as osimertinib which binds EGFR irreversibly, the compounds of the disclosure are non-covalent inhibitors.

In a first embodiment, the present disclosure provides a compound represented by the following structural formula (Ia):

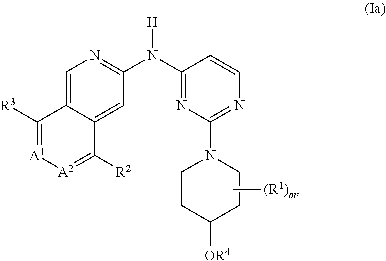

each A¹ and A² is independently N or CR; wherein each R is independently H, halogen, or CH₃;
each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ or in the group represented by R¹ is optionally substituted with 1 to 3 groups selected from deuterium, halogen, OH, NR^{a}R^{b}, C₁-C₂ alkyl, and C₁-C₂ alkoxy; or two R¹, attached to the same carbon atom, together with the carbon atom to which they are both attached form a C₃-C₄cycloalkyl optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is H, halogen, C₁-C₆ alkyl, C₁-C₆ alkoxy, C₃-C₆ cycloalkyl, 4 to 8 membered heterocyclyl, or 5 to 12-membered heteroaryl, wherein the alkyl, alkoxy, cycloalkyl, or heterocyclyl represented by R² is optionally substituted with 1 to 3 groups selected from R^{2a};
Each R^{2a} is independently selected from halogen, CN, OH, C(O)NR^{a}R^{b}, C₁-C₄ alkyl, C₁-C₄ alkoxy, and 4 to 8 membered heterocyclyl, wherein the alkoxy represented by R^{2a} is optionally substituted with 4 to 8 membered heterocyclyl, and the heterocyclyl represented by R^{2a} or in the group represented by R^{2a} is optionally substituted with C₁-C₄alkyl;
R³ is C₁-C₄ alkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is C₃-C₆cycloalkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is 5 to 12 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O;
R³ is a 4 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O; or
R³ is 5 to 12-membered heteroaryl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O;
R⁴ is H, or C₁-C₄alkyl optionally substituted with 1 to 3 groups selected from deuterium, OR^{a}, and NR^{a}R^{b}, or R⁴, and an R¹ attached to the same carbon atom, together with their intervening atoms form a 3 to 5 membered heterocyclyl;
each R^{a} and R^{b} is independently H or C₁-C₄ alkyl optionally substituted with 1 to 3 groups selected from deuterium, halogen, OH and NH₂; and
each R^{c} is independently C₁-C₄ alkyl optionally substituted with 1 to 3 halogen.

In some embodiments, the present disclosure provides a compound represented by the structural Formula (I), wherein each A¹ and A² are each independently N or CR and A³ is CR; wherein each R is independently H, halogen, or CH₃. In some embodiments, the compound is a compound of Formula (I), wherein A³ is CR and A¹ and A² are both CR or one or one of A¹ and A² is N and one of A¹ and A² is CR; wherein each R is independently H, halogen, or CH₃. In some embodiments, the compound is a compound of Formula (I), wherein A³ is CR and A¹ and A² are both CR, wherein each R is independently H, halogen, or CH₃. In some embodiments, the compound is a compound of Formula (I), wherein A³ is CR and A¹ is N and A² is CR; wherein each R is independently H, halogen, or CH₃. In some embodiments, the compound is a compound of Formula (I), wherein A³ is CR and A² is N and A¹ is CR; wherein each R is independently H, halogen, or CH₃. In some embodiments, the compound is a compound of Formula (I), wherein A³ is CH and A² is CH and A¹ is N. In some embodiments, the compound is a compound of Formula (I), wherein A³ is CH and A² is CH and A¹ is CH.

In some embodiments, the present disclosure provides a compound represented by the structural Formula (I) or (Ia), wherein each R¹ is independently halogen, OH, or C₁-C₄ alkyl and m is 0, 1, 2, 3, 4, 5, or 6. In some embodiments, a compound is a compound of Formula (I), wherein each R¹ is independently halogen, OH, or C₁-C₄ alkyl and m is 3, or 4. In some embodiments, a compound is a compound of Formula (I) or (Ia), wherein each R¹ is independently F, OH, or methyl and m is 3, or 4.

In some embodiments, the present disclosure provides a compound represented by the structural Formula (I) or (Ia), wherein one or more R¹ is C₁-C₄ alkoxy optionally substituted with 1 to 3 groups selected from deuterium, halogen, OH, NR^{a}R^{b}, C₁-C₂ alkyl, and C₁-C₂ alkoxy.

In some embodiments, the present disclosure provides a compound represented by the structural Formula (I) or (Ia), wherein one or more R¹ is C₃-C₆ cycloalkyl or —O—C₃-C₆ cycloalkyl, optionally substituted with 1 to 3 groups selected from deuterium, halogen, OH, NR^{a}R^{b}, C₁-C₂ alkyl, and C₁-C₂ alkoxy.

In some embodiments, the present disclosure provides a compound represented by the structural Formula (I) or (Ia), wherein each R¹ is independently halogen, CN, OH, NR^{a}R^{b}, C₁-C₄ alkyl, or C₁-C₄ alkoxy, wherein the alkyl or alkoxy represented by R¹ is optionally substituted with 1 to 3 groups selected from deuterium, halogen, OH; or two R¹, attached to the same carbon atom, together with the carbon atom to which both are attached form a C₃-C₄cycloalkyl; and/or m is 0, 1, 2, 3, or 4.

In some embodiments, the present disclosure provides a compound represented by the structural Formula (I) or (Ia), wherein each R¹ is independently halogen, OH, C₁-C₄ alkyl, or C₁-C₄ alkoxy, wherein the alkyl or alkoxy represented by R¹ is optionally substituted with 1 to 3 groups selected from OH; or two R¹, attached to the same carbon atom, together with the carbon atom to which both are attached form a C₃-C₄cycloalkyl; and/or m is 0, 1, 2, or 3.

In some embodiments, the present disclosure provides a compound represented by the structural Formula (I) or (Ia), wherein R² is C₁-C₄ alkyl, optionally substituted with 1 to 3 groups selected from halogen, CN, OH, C(O)NR^{a}R^{b}, and C₁-C₄ alkoxy, wherein the alkoxy is optionally substituted with 4 to 8 membered heterocyclyl optionally substituted with C₁-C₄alkyl. In some embodiments, the present disclosure provides a compound represented by the structural Formula (I) or (Ia), wherein R² is unsubstituted C₁-C₄ alkyl. In some embodiments, the present disclosure provides a compound represented by the structural Formula (I) or (Ia), wherein R² is C₃ alkyl. In some embodiments, the present disclosure provides a compound represented by the structural Formula (I) or (Ia), wherein R² is isopropyl.

In some embodiments, the present disclosure provides a compound represented by the structural Formula (I) or (Ia), wherein R² is H, halogen, or C₁-C₄ alkoxy, wherein the alkoxy represented by R² is optionally substituted with 1 to 3 groups selected from halogen, CN, OH, C(O)NR^{a}R^{b}, and C₁-C₄ alkoxy, wherein the alkoxy is optionally substituted with 4 to 8 membered heterocyclyl optionally substituted with C₁-C₄alkyl.

In some embodiments, the present disclosure provides a compound represented by the structural Formula (I) or (Ia), wherein R² is C₃-C₆ cycloalkyl optionally substituted with 1 to 3 groups selected from halogen, CN, OH, C(O)NR^{a}R^{b}, and C₁-C₄ alkoxy, wherein the alkoxy is optionally substituted with 4 to 8 membered heterocyclyl optionally substituted with C₁-C₄alkyl.

In some embodiments, the present disclosure provides a compound represented by the structural Formula (I) or (Ia), wherein R² is 4 to 8 membered heterocyclyl optionally substituted with 1 to 3 groups selected from halogen, CN, OH, C(O)NR^{a}R^{b}, and C₁-C₄ alkoxy, wherein the alkoxy is optionally substituted with 4 to 8 membered heterocyclyl optionally substituted with C₁-C₄alkyl.

In some embodiments, the present disclosure provides a compound represented by the structural Formula (I) or (Ia), wherein R² is 5 to 12-membered heteroaryl optionally substituted with 1 to 3 groups selected from halogen, CN, OH, C(O)NR^{a}R^{b}, and C₁-C₄ alkoxy, wherein the alkoxy is optionally substituted with 4 to 8 membered heterocyclyl optionally substituted with C₁-C₄alkyl.

In some embodiments, the present disclosure provides a compound represented by the structural Formula (I) or (Ia), wherein R² is C₁-C₆ alkyl, C₁-C₆ alkoxy, C₃-C₆ cycloalkyl, 4 to 6 membered heterocyclyl, or 5 to 6-membered heteroaryl, wherein the alkyl, alkoxy, cycloalkyl, or heterocyclyl represented by R² is optionally substituted with 1 to 3 groups selected from R^{2a}; and each R^{2a} is independently selected from halogen, CN, OH, C(O)NR^{a}R^{b}, C₁-C₄ alkyl, C₁-C₄ alkoxy, and 4 to 6 membered heterocyclyl, wherein the alkoxy represented by R^{2a} is optionally substituted with 4 to 6 membered heterocyclyl, and the heterocyclyl represented by R^{2a} or in the group represented by R^{2a} is optionally substituted with C₁-C₄alkyl.

In some embodiments, the present disclosure provides a compound represented by the structural Formula (I) or (Ia), wherein R² is C₁-C₆ alkyl, C₁-C₆ alkoxy, C₃-C₆ cycloalkyl, 4 to 6 membered heterocyclyl, or 5 to 6-membered heteroaryl, wherein the alkyl, alkoxy, cycloalkyl, or heterocyclyl represented by R² is optionally substituted with 1 to 3 groups selected from R^{2a}; and each R^{2a} is independently selected from halogen, CN, OH, C(O)NR^{a}R^{b}, C₁-C₄ alkoxy, and 4 to 6 membered heterocyclyl, wherein the alkoxy represented by R^{2a} is optionally substituted with 4 to 6 membered heterocyclyl, and the heterocyclyl represented by R^{2a} or in the group represented by R^{2a} is optionally substituted with C₁-C₄alkyl.

In some embodiments, the present disclosure provides a compound represented by the structural Formula (I) or (Ia), wherein R³ is C₁-C₄ alkyl optionally substituted with 1 to 3 groups selected from halogen, OR^{a}, ═O, CN, C(O)R^{c}, C(O)OR^{a}, 4 to 6-membered heterocycyl optionally substituted with 1 to 3 groups selected from halogen, deuterium, OR^{a}, CN, ═O, and C(O)R^{c}.

In some embodiments, the present disclosure provides a compound represented by the structural Formula (I) or (Ia), wherein R³ is C₁-C₄ alkyl optionally substituted with 1 to 3 groups selected from 4 to 6-membered heterocycyl optionally substituted with C(O)R^{c}.

In some embodiments, the present disclosure provides a compound represented by the structural Formula (I) or (Ia), wherein R³ is C₃-C₆cycloalkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, and ═O.

In some embodiments, the present disclosure provides a compound represented by the structural Formula (I) or (Ia), wherein R³ is C₃-C₆cycloalkyl optionally substituted with 1 to 3 groups selected from S(O)₂CH₃ and C₁-C₄ alkyl optionally substituted with 1 S(O)₂R^{c}.

In some embodiments, the present disclosure provides a compound represented by the structural Formula (I) or (Ia), wherein R³ is 5 to 10 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

In some embodiments, the present disclosure provides a compound represented by the structural Formula (I) or (Ia), wherein R³ is 5 to 10 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

In some embodiments, the present disclosure provides a compound represented by the structural Formula (I) or (Ia), wherein R³ is pyrrolidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

In some embodiments, the present disclosure provides a compound represented by the structural Formula (I) or (Ia), wherein R³ is 1,4,5,6-tetrahydropyrrolo[3,4-c]pyrazolyl, 1,6-diazaspiro[3.3]heptanyl, 1,6-diazaspiro[3.4]octanyl, 1,7-diazaspiro[3.5]nonanyl, 1-oxa-5-azaspiro[3.3]heptanyl, 1-thia-6-azaspiro[3.3]heptanyl, 2,6-diazaspiro[3.4]octanyl, 5-azaspiro[2.3]hexanyl, 5-azaspiro[2.4]heptanyl, 6-oxa-1-azaspiro[3.3]heptanyl or 5-oxa-2,7-diazaspiro[3.4]octan-6-onyl, each of which are optionally substituted with 1 to 3 groups selected from halogen, ═O, C(O)OR^{a}, and C₁-C₄ alkyl optionally substituted OR^{a}.

In some embodiments, the present disclosure provides a compound represented by the structural Formula (I) or (Ia), wherein R³ is a 4 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C1-C₄alkyl, and ═O.

In some embodiments, the present disclosure provides a compound represented by the structural Formula (I) or (Ia), wherein R³ is a azetidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O.

In some embodiments, the present disclosure provides a compound represented by the structural Formula (I) or (Ia), wherein R³ is a azetidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, triazolyl, diazolyl, oxadiazolyl, oxetanyl, and pyrrolidinonyl, wherein the triazolyl, diazolyl, oxadiazolyl, oxetanyl, and pyrrolidinonyl in the group represented by R³ are optionally substituted with 1 to 3 C₁-C₄alkyl.

In some embodiments, the present disclosure provides a compound represented by the structural Formula (I) or (Ia), wherein R³ is 5 or 6-membered heteroaryl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

In some embodiments, the present disclosure provides a compound represented by the structural Formula (I) or (Ia), wherein R³ is oxadiazolyl, pyrazolyl or triazolyl, each of which is optionally substituted with 1 to 3 groups selected from halogen and C₁-C₄ alkyl optionally substituted with halogen, OR^{a}, or NR^{a}R^{b}.

In some embodiments, the present disclosure provides a compound represented by the structural Formula (I) or (Ia), wherein R⁴ is H. In some embodiments, the present disclosure provides a compound represented by the structural Formula (I) or (Ia), wherein R⁴ is C₁-C₄alkyl, wherein the alkyl is optionally substituted with 1 to 3 groups selected from deuterium, OR^{a}, and NR^{a}R^{b}, or R⁴ and an R¹ attached to the same carbon together with their intervening atoms, form a 3 to 5 membered heterocyclyl. In some embodiments, the present disclosure provides a compound represented by the structural Formula (I) or (Ia), wherein R⁴ is methyl.

In some embodiments, the present disclosure provides a compound represented by the structural Formula (I) or (Ia), wherein R⁴ is H, or C₁-C₄alkyl optionally substituted with 1 to 3 groups selected from deuterium, OR^{a}, and NR^{a}R^{b}, or R⁴ and an R¹ attached to the same carbon atom together with their intervening atoms form an oxetanyl.

In some embodiments, the present disclosure provides a compound represented by the structural Formula (I) or (Ia), wherein each R^{a} and R^{b} is independently H or C₁-C₄ alkyl, wherein the alkyl is optionally substituted with 1 to 3 groups selected from deuterium, halogen, OH and NH₂. In some embodiments, the present disclosure provides a compound represented by the structural Formula (I) or (Ia), wherein each R^{a} and R^{b} is independently H or methyl. In some embodiments, the present disclosure provides a compound represented by the structural Formula (I) or (Ia), wherein R^{c} is methyl or ethyl each optionally substituted with 1 to 3 halogen.

In some embodiments, the present disclosure provides a compound represented by the structural Formula (I) or (Ia), wherein each R^{a} and R^{b} is independently H or C₁-C₂ alkyl optionally substituted with 1 to 3 groups selected from deuterium, halogen, OH and NH₂; and each R^{c} is independently C₁-C₂ alkyl optionally substituted with 1 to 3 halogen.

In some embodiments, the present disclosure provides a compound represented by the structural Formula (I) or (Ia), wherein each R^{c} is independently C₁-C₄ alkyl, wherein the alkyl is optionally substituted with 1 to 3 halogen. In some embodiments, the present disclosure provides a compound represented by the structural Formula (I) or (Ia), wherein each R^{c} is independently C₁-C₄ alkyl, wherein the alkyl is optionally substituted with 1 to 3 F. In some embodiments, the present disclosure provides a compound represented by the structural Formula (I) or (Ia), wherein each R^{c} is independently methyl.

In some embodiments, a compound is a compound of Formula (IIa), or a pharmaceutically acceptable salt thereof,

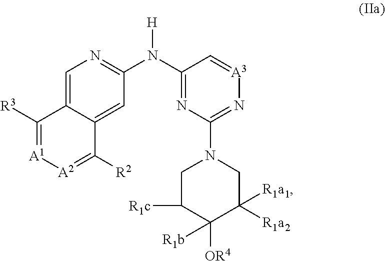

wherein each R₁ₐ₁, R₁ₐ₂, R_{1b} and R_{1c} are each independently H, halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ or in the group represented by R¹ is optionally substituted with 1 to 3 groups selected from deuterium, halogen, OH, NR^{a}R^{b}, C₁-C₂ alkyl, and C₁-C₂ alkoxy.

In some embodiments, a compound is a compound of Formula (IIa), wherein R₁ₐ₁ is hydrogen or C1-C₄ alkyl, R₁ₐ₂ is halogen, C₁-C₄ alkoxy optionally substituted with OH, or OH, R_{1b} is hydrogen or C₁-C₄ alkyl optionally substituted with OH, R⁴ is hydrogen or C₁-C₄ alkyl optionally substituted with OH, R_{1c} is hydrogen or halogen, and R³, A¹, A², A³, and R² are as defined above with respect to Formula (I).

In some embodiments, a compound is a compound of Formula (IIa), wherein R₁ₐ₁ is methyl, R₁ₐ₂ is F, R_{1b} is hydrogen, R⁴ is hydrogen R_{1c} is hydrogen, and R³, A¹, A², A³, and R² are as defined above with respect to Formula (I).

In some embodiments, a compound is a compound of Formula (IIa), wherein R₁ₐ₁ is methyl, R₁ₐ₂ is F, R_{1b} is hydrogen, R⁴ is methyl R_{1c} is hydrogen, and R³, A¹, A², A³, and R² are as defined above with respect to Formula (I).

In some embodiments, a compound is a compound of Formula (IIa), wherein R₁ₐ₁ is hydrogen, R₁ₐ₂ is F, R_{1b} is methyl, R⁴ is hydrogen, R_{1c} is hydrogen, and R³, A¹, A², A³, and R² are as defined above with respect to Formula (I).

In some embodiments, a compound is a compound of Formula (IIa), wherein R₁ₐ₁ is hydrogen, R₁ₐ₂ is F, R_{1b} is hydrogen, R⁴ is methyl, R_{1c} is hydrogen, and R³, A¹, A², A³, and R² are as defined above with respect to Formula (I).

In some embodiments, a compound is a compound of Formula (IIa), wherein R₁ₐ₁ is hydrogen, R₁ₐ₂ is F, R_{1b} is hydrogen, R⁴ is C₁-C₄ alkyl substituted with OH, R_{1c} is hydrogen, and R³, A¹, A², A³, and R², and R⁴ are as defined above with respect to Formula (I). In some embodiments, a compound is a compound of Formula (IIa), wherein R₁ₐ₁ is hydrogen, R₁ₐ₂ is F, R_{1b} is hydrogen, R⁴ is —CH₂CH₂OH, R_{1c} is hydrogen, and R³, A¹, A², A³, and R² are as defined above with respect to Formula (I).

In some embodiments, a compound is a compound of Formula (IIa), wherein R₁ₐ₁ is hydrogen, R₁ₐ₂ is F, R_{1b} is hydrogen, R⁴ is C₁-C₄ alkyl substituted with NR^{a}R^{b}, R_{1c} is hydrogen, and R^{a}, R^{b}, R³, A¹, A², A³, and R² are as defined above with respect to Formula (I). In some embodiments, a compound is a compound of Formula (IIa), wherein R₁ₐ₁ is hydrogen, R₁ₐ₂ is F, R_{1b} is hydrogen, R⁴ is —CH₂CH₂NHCH₃, R_{1c} is hydrogen, and R³, A¹, A², A³, and R² are as defined above with respect to Formula (I).

In some embodiments, a compound is a compound of Formula (II),

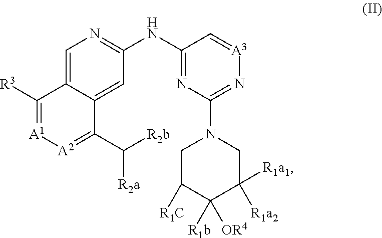

or a pharmaceutically acceptable salt thereof, wherein R₁ₐ₁ is H, halogen, or C₁-C₄alkyl optionally substituted with OH; R₁ₐ₂ is H, halogen, OH, or C1-C₄alkyl; or R₁ₐ₁ and R₁ₐ₂ together form a C₃-C₄cycloalkyl; R₂ₐ is H, CN, oxetane, or C₁-C₃alkyl optionally substituted with CN, OH or methoxy; R_{2b} is H or methyl; or R₂ₐ and R_{2b} together form a 3-4-member cycloalkyl or 4-6-member heterocyclyl, wherein the cycloalkyl or heterocyclyl is optionally substituted with C₁-C₄alkyl; R_{1b} is H or C₁-C₄alkyl optionally substituted with OH; R_{1c} is H or halogen; and R⁴ is H or C₁-C₄alkyl optionally substituted with 1 to 3 deuterium, OH or NR^{a}R^{b}; or R_{1b} and R₄ together form a 3 to 5-member heterocyclyl ring. In some embodiments, R₂ₐ and R_{2b} are each methyl. In some embodiments, R₁ₐ₁ is H or methyl and R₁ₐ₂ is F; R₂ₐ is methyl or methylene substituted with CN; R_{2b} is methyl; R_{1b} and R₄ are each independently H or methyl; or R_{1b} and R₄ together form an oxetane ring; and R_{1c} is H or F. In some embodiments, R₁ₐ₁ is methyl, R₁ₐ₂ is F, R_{1b} is H and R₄ is H or CH₃; R₁ₐ₁ is H, R₁ₐ₂ is F and R_{1b} is CH₃; R_{1b} and R₄ together form an oxetane ring; R₁ₐ₁ is H, R₄ is H and R_{1b} is CH₃; or R⁴ is CH₃, and R₁ₐ₁ is H. In some embodiments, A² is CH, R₁ₐ₁ is methyl, R₁ₐ₂ is F, R_{1b} is H, R_{1c} is hydrogen, R₂ₐ and R_{2b} are both methyl, and R⁴ is H. In one embodiment, a compound of the present disclosure is any one of the compounds disclosed in the examples and Table 1, or a pharmaceutically acceptable salt thereof.

In one embodiment, the compounds in Table 3 and pharmaceutically acceptable salts thereof are excluded from the disclosure.

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds disclosed herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” in the Experimental section refers to an intended reaction product compound obtained from a chromatography separation/purification that elutes earlier than a second intended reaction product compound from the same preceding reaction. The second intended product compound is referred to as “peak 2”.

When a disclosed compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are selective EGFR inhibitors. As used herein, the term “selective EGFR inhibitor” means a compound which selectively inhibits certain mutant EGFR kinases over wild-type EGFR and the kinome. Said another way, a selective EGFR inhibitor has no or low activity against wild-type EGFR and the kinome. A selective EGFR inhibitor's inhibitory activity against certain mutant EGFR kinases is more potent in terms of IC₅₀ value (i.e., the IC₅₀ value is subnanomolar) when compared with its inhibitory activity against wild-type EGFR and many other kinases. Potency can be measured using known biochemical assays.

Some compounds of the disclosure have the advantage of good penetration of the brain. The ability of a particular compound to cross the BBB and penetrate the brain can be assessed using a variety of known methods or combinations of such methods. One in vitro method that is frequently used to predict a compound's in vivo brain penetration is P-gp efflux ratio. P-glycoprotein (P-gp) is expressed at the blood-brain barrier (BBB) and restricts the penetration of its substrates into the central nervous system (CNS). Compounds that are found to be good P-gp substrates in vitro (i.e., have a high efflux ratio) are predicted to have poor in vivo brain penetration. In order to measure the P-gp efflux ratio, Madin-Darby canine kidney cells overexpressing P-gp (MDCK-MDR1 cells) the apparent apical to basolateral permeability (Papp[A-B]) and the apparent basolateral to apical permeability (Papp[B-A]) for compounds is determined. The P-gp efflux ratio is a measure of the ratio of Papp[B-A]/Papp[A-B]. In some embodiments, a compound of the disclosure has a P-gp efflux ratio of less than 2, less than 3, less than 4, less than 5.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes. Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (I)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The present disclosure provides a method of inhibiting certain mutant forms of epidermal growth factor receptor (EGFR) in a subject in need thereof, comprising administering to the subject an effective amount of a compound disclosed herein, a pharmaceutically acceptable salt thereof or a pharmaceutical composition disclosed herein. Mutant forms of EGFR include for example, EGFR with LRTMCS mutation (the exon 19 deletion (del19) or exon 21 (L858R) substitution mutation, T790M mutation, and C797S mutation). Subjects “in need of inhibiting EGFR” are those having a disease for which a beneficial therapeutic effect can be achieved by inhibiting at least one mutant EGFR, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by mutant EGFR, wherein the inhibition of the mutant EGFR is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. Cancers to be treated according to the disclosed methods include lung cancer, colon cancer, urothelial cancer, breast cancer, prostate cancer, brain cancers, ovarian cancer, gastric cancer, pancreatic cancer, head and neck cancer, bladder cancer, and mesothelioma, including metastasis (in particular brain metastasis) of all cancers listed. Typically, the cancer is characterized by at one or more EGFR mutations described herein. In a specific embodiment, the cancer has progressed on or after EGFR tyrosine kinase inhibitor (TKI) Therapy. In a specific embodiment, the disease has progressed on or after first line osimertinib.

In a specific embodiment, the cancer to be treated is lung cancer. In a more specific embodiment, the cancer is non-small cell lung cancer (NSCLC). In some embodiments, the lung cancer is locally advanced or metastatic NSCLC, NSCLC adenocarcinoma, NSCLC with squamous histology and NSCLC with non-squamous histology. In another embodiment, the lung cancer is NSCLC adenocarcinoma. In another specific embodiment, the lung cancer (or non-small cell lung cancer) has metastasized to the brain.

In another embodiment, the disease/condition/or cancer associated with or modulated by mutant EGFR that is characterized by an EGFR genotype selected from genotypes 1-17 according the Table below (del18=Exon 18 deletion, specifically, e.g., del E709_T710 insD; del19=Exon 19 deletion, specifically, e.g., delE746_A750 (most common), delE746_S752insV, de1747_A750insP, delL747_P753insS, and delS752_I759; ex20ins—Exon 20 insertion, specifically, e.g., D761-E762insX, A763-Y764insX, Y764-V765insX, V765-M766insX, A767-S768insX, S768-D769insX, V769-D770insX, N771-P772insX, P772-H773insX, H773-V774insX, and V774-C775insX):

### EGFR Genotype

| | |
|---|---|
| 1 | EGFR del19 |
| 2 | EGFR del19 T790M |
| 3 | EGFR del19 C797S |
| 4 | EGFR del19 C797X (C797G or C797N) |
| 5 | EGFR del19 T790M C797S |
| 6 | EGFR del19 T790M C797S Q791P |
| 7 | EGFR del19 T790M (C797G or C797N) |
| 8 | EGFR del19 L792X (L792F, L792H or L792Y) |
| 9 | EGFR del19 T790M L792X (L792F, L792H, or L792Y) |
| 10 | EGFR del19 G796R (G796S) |
| 11 | EGFR del19 T790M G796R (G796S) C797S L792X |
| | (L792F, L792H or L792Y) |
| 12 | EGFR del19 L792R (L792V or L792P) |
| 13 | EGFR del19 L718Q (L718V) |
| 14 | EGFR del19 T790M L718Q (L718V) L792X (L792F, |
| | L792H or L792Y) |
| 15 | EGFR del19 T790M G796R (G796S) |
| 16 | EGFR del19 T790M L792R (L792V or L792P) |
| 17 | EGFR del19 T790M L718Q (L718V) |
| 18 | EGFR del19 T790M C797S L718Q (L718V) |
| 19 | EGFR del19 G724S |
| 20 | EGFR del19 T790M G724S |
| 21 | EGFR del19 S768I (SV768IL) |
| 22 | EGFR del19 T790M S768I (SV768IL) |
| 23 | EGFR del19 T790M C797S/G L792X (L792F, L792H, |
| | L792R, or L792Y) |
| 24 | EGFR del 19 V834L |
| 25 | EGFR del 19 T790M V834L |
| 27 | EGFR del19 T790M L792X (L792F, L792H, L792R, or |
| | L792Y) |
| 28 | EGFR del19 C797S L718Q (L718V) |
| 29 | EGFR del19 L718Q (L718V) A750P |
| 30 | EGFR del19 T790M L718Q (L718V) A750P L792V |
| | G796R |
| 31 | EGFR L858R |
| 32 | EGFR L858R T790M |
| 33 | EGFR L858R C797S |
| 34 | EGFR L858R C797X (797G or C797N) |
| 35 | EGFR L858R T790M C797S |
| 36 | EGFR L858R T790M C797S Q791P |
| 37 | EGFR L858R T790M C797X (C797G or C797N) |
| 38 | EGFR L858R L792X (L792F, L792H or L792Y) |
| 39 | EGFR L858R T790M L792X (L792F, L792H or L792Y) |
| 40 | EGFR L858R G796R (G796S) |
| 41 | EGFR L858R T790M G796R (G796S) C797S L792X |
| | (L792F, L792H or L792Y) |
| 42 | EGFR L858R L792R (L792V or L792P) |
| 43 | EGFR L858R L718Q (L718V) |
| 44 | EGFR L858R T790M G796R (G796S) |
| 45 | EGFR L858R T790M L792R (L792V or L792P) |
| 46 | EGFR L858R T790M L718Q (L718V) |
| 47 | EGFR L858R T790M C797S L718Q (L718V) |
| 48 | EGFR L858R T790M L718Q (L718V) L792X (L792F, |
| | L792H or L792Y) |
| 49 | EGFR L858R G724S |
| 50 | EGFR L858R T790M G724S |
| 51 | EGFR L858R S768I (SV768IL) |
| 52 | EGFR L858R T790M S768I (SV768IL) |
| 53 | EGFR L858R T790M C797S/G L792X (L792F, L792H, |
| | L792R, or L792Y) |
| 54 | EGFR L858R V834L |
| 55 | EGFR L858R T790M V834L |
| 57 | EGFR L858R T790M L792X (L792F, L792H, L792R, or |
| | L792Y) |
| 58 | EGFR L858R C797S L718Q (L718V) |
| 59 | EGFR L858R L718Q (L718V) A750P |
| 60 | EGFR L858R T790M L718Q (L718V) A750P L792V |
| | G796R |
| 61 | EGFR L861Q |
| 62 | EGFR L861Q T790M |
| 63 | EGFR L861Q T790M C797S/G/N |
| 64 | EGFR L861Q C797S/G/N |
| 65 | EGFR del18 |
| 66 | EGFR G719X (G719A, G719S, G719C, G719R, G719D, |
| | or G719V) |
| 67 | EGFR E709X (E709K, E709H, or E709A) |
| 68 | EGFR E709X (E709K, E709H, or E709A) (G719A, |
| | G719S, G719C, G719D, G719R, or G719V) |
| 69 | EGFR G719X (G719A, G719S, G719C, G719D, G719R, |
| | or G719V) S768I |
| 70 | EGFR ex20ins |
| 71 | EGFR ex20ins L718Q |
| 72 | EGFR ex20ins T790M |
| 73 | EGFR ex20ins C797S |
| 74 | EGFR S7681I |
| 75 | EGFR T790M |
| 76 | EGFR T790M C797S/G L792X (L792F, L792H, L792R, |
| | or L792Y) |

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797X (C797G or C797N).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M (C797G or C797N).

In another embodiment, the disease/condition/or cancer (e.g., NSCLC) being treated with a disclosed compound, a pharmaceutically acceptable salt, or a pharmaceutical composition disclosed herein is characterized by EGFR comprising EGFR del19 L792X (L792F, L792H or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 T790M L792X (L792F, L792H, or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 G796R (G796S).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L792R (L792V or L792P).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L718Q (L718V).

In another embodiment, the disease/condition/or cancer (e.g., NSCLC) being treated with a disclosed compound, a pharmaceutically acceptable salt thereof, or pharmaceutical composition described herein is characterized by EGFR comprising EGFR del19 T790M G796R (G796S).

In another embodiment, the disease/condition/or cancer (e.g., NSCLC) being treated with a disclosed compound, a pharmaceutically acceptable salt thereof, or pharmaceutical composition described herein is characterized by EGFR comprising EGFR del19 T790M L792R (L792V or L792P).

In another embodiment, the disease/condition/or cancer (e.g., NSCLC) being treated with a disclosed compound, a pharmaceutically acceptable salt thereof or pharmaceutical composition described herein is characterized by EGFR comprising EGFR del19 T790M L718Q (L718V).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797X (797G or C797N).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M C797X (797G or C797N).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L792X (L792F, L792H or L792Y).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L790M L792X (L792F, L792H or L792Y).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L718Q (L718V).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M L718Q (L718V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del18.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719R, G719D, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A) (G719A, G719S, G719C, G719D, G719R, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719D, G719R, or G719V) S7681.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins L718Q.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR S7681I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR T790M C797S/G L792X (L792F, L792H, L792R, or L792Y).

In another embodiment, the disease/condition/or cancer (e.g., NSCLC) being treated with a disclosed compound, a pharmaceutically acceptable salt or a pharmaceutical composition disclosed herein is characterized by an EGFR genotype selected from genotypes 1-76.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to gefitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to erlotinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and gefitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and erlotinib.

Another embodiment is the treatment a subject with metastatic NSCLC with tumors harboring activating Exon 19 Deletion or L858R EGFR mutations as well as a resistance mutation disclosed herein as detected by an approved molecular testing methodology. Another embodiment is a disclosed compound used in combination with a 1^{st} or 3^{rd} generation TKI indicated for the treatment of subject with metastatic NSCLC with tumors harboring T790M and C797S mutations as detected by an approved test, and whose disease has progressed on or after at least 2 prior EGFR TKI therapies.

Another embodiment is a disclosed compound for the treatment of subjects with metastatic NSCLC whose disease with on-target EGFR resistance has progressed on or after any EGFR TKI. In a specific embodiment, the disclosed compound is used in combination with a 1^{st} or 3^{rd} generation TKI indicated for the treatment of subject with metastatic NSCLC.

Another embodiment is a disclosed compound for the treatment of subjects with metastatic EGFR C797S mutation-positive NSCLC as detected by an approved molecular test, whose disease has progressed on or after first-line osimertinib. In a specific embodiment, the disclosed compound is used in combination with a 1^{st} or 3^{rd} generation TKI indicated for the treatment of subject with metastatic NSCLC.

In a particular embodiment, the deletions, mutations, and insertions disclosed herein are detected by an FDA-approved test.

A person of ordinary skill in the art can readily determine the certain EGFR alterations a subject possesses in a cell, cancer, gene, or gene product, e.g., whether a subject has one or more of the mutations or deletions described herein using a detection method selected from those known in the art such as hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect one or more EGFR deletions and/or mutations, a primary tumor sample, circulating tumor DNA (ctDNA), circulating tumor cells (CTC), and/or circulating exosomes may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples.

The compounds of the disclosure, pharmaceutically acceptable salts thereof or pharmaceutical compositions disclosed herein may be used for treating to a subject who has become refractory to treatment with one or more other EGFR inhibitors. “Refractory” means that the subject's cancer previously responded to drugs but later responds poorly or not at all. In some embodiments, the subject has become refractory to one or more first generation EGFR inhibitors such as erlotinib, gefitinib, icotinib or lapatinib. In some embodiments, the subject has been become refractory to treatment with one or more second generation EGFR inhibitors such as afatinib, dacomitinib, poziotinib, or neratinib. In some embodiments the subject has become refractory to treatment with one or more first generation inhibitors and one or more second generation inhibitors.

In some embodiments, the subject has become refractory to treatment with one or more third generation inhibitors such as osimertinib, nazartinib, or avitinib. In one embodiment, the subject has become refractory to treatment with one or more first generation EGFR inhibitors and one or more third generation EGFR inhibitors. In some embodiments, the subject has become refractory to treatment with one or more second generation EGFR inhibitors and one or more third generation EGFR inhibitors. In some embodiments, the subject has become refractory to treatment with one or more first generation inhibitors, and one or more third generation EGFR inhibitors.

### Combinations

The compounds of the disclosure, pharmaceutically acceptable salts thereof, or pharmaceutical compositions disclosed herein can be used in combination with one or more additional pharmacologically active substances. For example, the disclosure includes methods of treating a condition/disease/or cancer comprising administering to a subject in need thereof a compound of the disclosure or a pharmaceutically acceptable salt or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alflutinib (AST 2818), almonertinib (HS10296), BBT-176, BI-4020, CH7233163, gilitertinib, JND-3229, lazertinib, nazartinib (EGF 816), PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). For the treatment of cancer e.g., NSCLC using a compound of the disclosure or pharmaceutically acceptable salt thereof or pharmaceutical composition disclosed herein in combination with a first line therapy, for example a first, second, or third generation EGFR inhibitor (i.e., as an initial treatment before the cancer has become refractory) may forestall or delay the cancer from becoming refractory. Typically, the cancer is characterized by one of the EGFR genotypes described herein.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib, glumetinib, tepotinib) and MET antibodies (emibetuzumab, telisotuzumab vedotin (ABBV 339)); mitotic kinase inhibitors (CDK4/6 inhibitors such as palbociclib, ribociclib, abemacicilb, GIT38); anti-angiogenic agents e.g., bevacizumab, nintedanib; apoptosis inducers such as Bcl-2 inhibitors e.g, venetoclax, obatoclax, navitoclax, palcitoclax (APG-1252), and Mcl-1 inhibitors e.g., AZD-5991, AMG-176, S-64315; mTOR inhibitors e.g, rapamycin, temsirolimus, everolimus, ridoforolimus; RET inhibitors, like pralsetinib and selpercatinib, and PI3K inhibitors dactolisib (BEZ235), pictilisib (GDC-0941), LY294002, idelalisib (CAL-101); JAK inhibitors (e.g., AZD4205, itacitinib), Aurora A inhibitors (e.g., alisertib); BCR/ABL and/or Src family tyrosine kinase inhibitors (e.g., dasatinib); VEGF inhibitors (e.g., MP0250; ramucirumab); multi-kinase protein inhibitors (e.g., anlotinib, midostaurin); PARP inhibitors (e.g., niraparib); platinum therapies (e.g., cisplatin (CDDP), carboplatin (CBDCA), or nedaplatin (CDGP)); PD-L1 inhibitors (e.g., durvalumab (MEDI 4736)); HER2/neu receptor inhibitors (e.g., trastuzumab); anti-HER2 or anti-HER3 antibody-drug conjugates (e.g., patritumab deruxtecan (U3-1402), trastuzumab emtansine); or immunogene therapy (e.g., oncoprex).

A “subject” is a human in need of treatment.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed EGFR inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The following examples are intended to be illustrative and are not intended to be limiting in any way to the scope of the disclosure.

### EXEMPLIFICATION

### Examples

### Preparation of Exemplary Compounds

### Definitions

Abbreviations and acronyms used herein include the following:
AcOH means acetic acid;
AIBN means 2,2′-azobis(2-methylpropionitrile);
Aq. means aqueous;
BBr₃ means boron tribromide;
BINAP means (±)-2,2′-Bis(diphenylphosphino)-1,1′-binaphthalene;
Bn means benzyl;
Boc means tert-butoxy carbonyl;
BOP means (Benzotriazol-1-yloxy)tris(dimethylamino)phosphonium hexafluorophosphate;
(BPin)₂ means 4,4,4′,4′,5,5,5′,5′-Octamethyl-2,2′-bi-1,3,2-dioxaborolane;
br means broad;
BrettPhos Pd G3 or BrettP Pd G3 means [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate;
n-Bu₄NI means Tetra-n-butylammonium iodide;
n-BuOH means butan-1-ol;
t-BuOH means tertiary butanol;
t-BuOK means potassium tert-butoxide;
° C. means degrees Celsius;
cataCXium® A means Di(1-adamantyl)-n-butylphosphine;
CDCl₃ means deutero-chloroform;
CDI means 1,1′-Carbonyldiimidazole;
CPhos means 2-Dicyclohexylphosphino-2′,6′-bis(N,N-dimethylamino)biphenyl;
Cs₂CO₃ means cesium carbonate;
CuI means copper iodide;
δ means chemical shift;
d means doublet;
dd means double of doublets;
DAST means Diethylaminosulfur trifluoride;
DBU means 1,8-Diazabicyclo[5.4.0]undec-7-ene;
DCM means dichloromethane;
DCE means 1,2-dichloroethane;
DEA means diethylamine;
DEAD means diethyl azodicarboxylate;
DIAD means diisopropyl azodicarboxylate;
DIPEA means N-ethyldiisopropylamine or N,N-diisopropylethylamine;
DMA means N,N-Dimethylacetamide;
DMAP means 4-(Dimethylamino)pyridine;
DMF means N,N-dimethylformamide;
DMSO means Dimethylsulfoxide;
DMSO-d₆ means hexadeuterodimethyl sulfoxide;
EDC·HCl and EDC means N-Ethyl-N′-(3-dimethylaminopropyl)carbodiimide hydrochloride;
Et means ethyl;
Et₂O means diethyl ether;
EtOH means ethanol;
EtOAc means ethyl acetate;
Eq. means equivalent;
g means gram;
HATU means 1-[bis(dimethylamino)methylene]-1H-1,2,3-triazolo[4,5-b]pyridinium 3-oxid hexafluorophosphate;
HBTU means N,N,N′,N′-Tetramethyl-O-(1H-benzotriazol-1-yl)uronium hexafluorophosphate;
HCl means hydrochloric acid;
HCOH means formaldehyde;
HCO₂H means formic acid
¹H NMR means proton nuclear magnetic resonance;
H₂O means water;
H₂O₂ means hydrogen peroxide;
HOBt means 1-Hydroxybenzotriazole hydrate;
HPLC means high pressure liquid chromatography;
h means hour;
IPA means 2-propanol;
K₂CO₃ means potassium carbonate;
KI means potassium iodide;
KOH means potassium hydroxide;
K₃PO₄ means potassium phosphate tribasic;
L means litre;
LCMS means liquid chromatography mass spectrometry;
LiCl means lithium chloride;
LiOH means lithium hydroxide;
LiAlH₄ means lithium aluminium hydride;
m means multiplet;
M means molar;
Me means methyl;
MeMgBr means methyl magnesium bromide;
MeCN means acetonitrile;
Mel means iodomethane;
MeLi means methyl lithium;
MeOH means methanol;
MeOH-d₄ means deutero-methanol;
mg means milligram;
MgSO₄ means magnesium sulfate;
MHz means mega Hertz;
mins means minutes;
mL means millilitres;
mmol means millimole;
MS m/z means mass spectrum peak;
MsCl means methanesulfonyl chloride;
MTBE means tert-butyl methyl ether;
N₂ means nitrogen;
NaBH₄ means sodium borohydride;
NaBH(OAc)₃ means sodium triacetoxyborohydride;
NaBH₃CN means sodium cyanoborohydride;
NaCN means sodium cyanide;
Na₂CO₃ means sodium carbonate;
NaH means sodium hydride;
NaHCO₃ means sodium bicarbonate;
NaOH means sodium hydroxide;
Na₂SO₄ means sodium sulfate;
Na₂SO₃ means sodium sulfite;
NBS means N-bromosuccinimide;
NH₃ means ammonia;
NH₄Cl means ammonium chloride;
NH₂NH₂ means hydrazine;
NH₄OH is ammonium hydroxide;
NIS means N-iodosuccinimide;
PE means petroleum ether;
Pd(amphos)Cl₂ means Bis(di-tert-butyl(4-dimethylaminophenyl)phosphine)dichloropalladium(II);
Pd(OAc)₂ means palladium acetate;
Pd₂(dba)₃ means tris(dibenzylideneacetone)dipalladium (0);
Pd(dppf)Cl₂ means [1,1′-bis(diphenylphosphino)ferrocene]dichloropalladium(II);
Pd(dtbpf)Cl₂ means [1,1′-Bis(di-tert-butylphosphino)ferrocene]dichloropalladium(II);
Pd(PPh₃)₄ means tetrakis(triphenylphosphine)palladium(0);
Pd/C means palladium on charcoal;
Pd(OH)₂ means palladium hydroxide;
PPh₃ means triphenylphosphine;
POCl₃ means phosphorous oxychloride;
PtBu₃HBF₄ means Tri-tert-butylphosphonium tetrafluoroborate;
PtO₂ means platinum (II) oxide;
q means quartet;
Rockphos Pd G3 means [(2-Di-tert-butylphosphino-3-methoxy-6-methyl-2′,4′,6′-triisopropyl-1,1′-biphenyl)-2-(2-aminobiphenyl)]palladium(II) methanesulfonate;
rt means room temperature;
RT means retention time;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat. means saturated;
Selectfluor means 1-chloromethyl-4-fluoro-1,4-diazoniabicyclo[2.2.2]octane bis(tetrafluoroborate;
SFC means supercritical fluid chromatography;
soln. means solution;
t means triplet;
tBuXPhos means 2-Di-tert-butylphosphino-2′,4′,6′-triisopropylbiphenyl;
TEA means triethylamine;
TESCl means chlorotriethylsilane;
TFA means trifluoroacetic acid;
Tf₂O means trifluoromethanesulfonic anhydride;
THF means tetrahydrofuran;
TLC means thin layer chromatography;
TsCl means p-toluenesulfonyl chloride;
μL means micro litres;
μmol means micromole;
Xantphos means 4,5-Bis(diphenylphosphino)-9,9-dimethylxanthene;
Xantphos Pd G2 means Chloro[(4,5-bis(diphenylphosphino)-9,9-dimethylxanthene)-2-(2′-amino-1,1′-biphenyl)]palladium(II);
Xantphos Pd G3 means [(4,5-Bis(diphenylphosphino)-9,9-dimethylxanthene)-2-(2′-amino-1,1′-biphenyl)]palladium(II) methanesulfonate;
XPhos Pd G2 means Chloro(2-dicyclohexylphosphino-2′,4′,6′-triisopropyl-1,1′-biphenyl)[2-(2′-amino-1,1′-biphenyl)]palladium(II);
XPhos Pd G3 means (2-dicyclohexylphosphino-2′,4′,6′-triisopropyl-1,1′-biphenyl)[2-(2′-amino-1,1′-biphenyl)]palladium(II) methanesulfonate; and
Zn(CN)₂ means zinc cyanide.

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization:

LC-MS: The liquid chromatography-mass spectrometry (LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 4 minutes was utilized. The flow rate was constant at 1 mL/min. Alternatively, the liquid chromatography-mass spectrometry (LC-MS) data (sample analyzed for purity and identity) were obtained with a Shimadzu LCMS system using an Shimadzu LCMS mass spectrometer utilizing ESI ionization fitted with an Agilent (Poroshel HPH-C18 2.7 um particle size, 3.0×50 mm dimensions) reverse-phase column at 22.4 degrees Celsius. The mobile phase consisted of a mixture of solvent 5 mM NH₄HCO₃ (or 0.05% TFA) in water and acetonitrile. A constant gradient from 90% aqueous/10% organic to 5% aqueous/95% organic mobile phase over the course of 2 minutes was utilized. The flow rate was constant at 1.5 mL/min.

Prep LC-MS: Preparative HPLC was performed on a Shimadzu Discovery VP® Preparative system fitted with a Luna 5u C18(2) 100A, AXIA packed, 250×21.2 mm reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 25 minutes was utilized. The flow rate was constant at 20 mL/min. Reactions carried out in a microwave were done so in a Biotage Initiator microwave unit. Alternatively, the preparative HPLC was performed on a Waters Preparative system fitted with Column: Xbridge Shield RP18 OBD Column, 30*150 mm, 5 um; The mobile phase consisted of a mixture of solvent Water (10 mmol/L NH₄HCO₃+0.05% NH3·H2O) and acetonitrile. A constant gradient from 95% aqueous/5% organic to 5% aqueous/95% organic mobile phase over the course of 11 minutes was utilized. The flow rate was constant at 60 mL/min. Reactions carried out in a microwave were done so in a Biotage Initiator microwave unit.

Silica gel chromatography: Silica gel chromatography was performed on a Teledyne Isco CombiFlash® Rf unit, a Biotage® Isolera Four unit, or a Biotage® Isolera Prime unit.

Proton NMR: ¹H NMR spectra were obtained with a Varian 400 MHz Unity Inova 400 MHz NMR instrument (acquisition time=3.5 seconds with a 1 second delay; 16 to 64 scans) or a Avance 400 MHz Unity Inova 400 MHz NMR instrument (acquisition time=3.99 seconds with a 1 second delay; 4 to 64 scans) or a Avance 300 MHz Unity Inova 300 MHz NMR instrument (acquisition time=5.45 seconds with a 1 second delay; 4 to 64 scans). Unless otherwise indicated, all protons were reported in DMSO-d6 solvent as parts-per million (ppm) with respect to residual DMSO (2.50 ppm).

SFC: Waters Preparative system.

Chiral-HPLC: Agilent 1260 Preparative system.

One of ordinary skill in the art will recognize that modifications of the gradient, column length, and flow rate are possible and that some conditions may be more suitable for compound characterization than others, depending on the chemical species being analyzed.

### Preparative HPLC Purification:

The following codes refer to the preparative HPLC conditions used as indicated in the examples and preparation sections. Individual gradients were optimised for each example as appropriate.

| Prep-HPLC | |
|---|---|
| Code | Conditions |
| HPLC-1 | Kinetex EVO C18, 30 × 150 mm, 5 mm; MeCN/H₂O (0.05% NH₄OH + |
| | 10 mM NH₄HCO₃) |
| HPLC-2 | Kinetex EVO C18, 30 × 150 mm, 5 mm; MeCN/H₂O (10 mM NH₄HCO₃) |
| HPLC-3 | SunFire Prep C18 OBD, 19 × 150 mm, 5 mm; MeCN/H₂O (+0.05% TFA) |
| HPLC-4 | SunFire Prep C18 OBD, 19 × 250 mm, 5 mm; MeCN/H₂O (+0.1% HCO₂H) |
| HPLC-5 | SunFire Prep C18 OBD, 19 × 250 mm, 5 mm; MeCN/H₂O (+0.1% TFA) |
| HPLC-6 | XBridge C18 OBD, 19 × 150 mm, 5 mm; MeCN/H₂O (+0.05% NH₄OH + |
| | 10 mM NH₄HCO₃) |
| HPLC-7 | XBridge C18 OBD, 19 × 150 mm, 5 mm; MeCN/H₂O (+0.1% NH₄OH + |
| | 10 mM NH₄HCO₃) |
| HPLC-8 | XBridge C18 OBD, 19 × 150 mm, 5 mm; MeCN/H₂O (+10 mM NH₄HCO₃) |
| HPLC-9 | XBridge C18 OBD, 19 × 250 mm, 10 mm; MeCN/H₂O (+0.1% NH₄OH + |
| | 10 mM NH₄HCO₃) |
| HPLC-10 | XBridge C18 OBD, 19 × 250 mm, 5 mm; MeCN/H₂O (+0.05% NH₄OH) |
| HPLC-11 | XBridge C18 OBD, 19 × 250 mm, 5 mm; MeCN/H₂O (+0.05% NH₄OH + |
| | 10 mM NH₄HCO₃) |
| HPLC-12 | XBridge C18 OBD, 19 × 250 mm, 5 mm; MeCN/H₂O (+0.1% NH₄OH + |
| | 10 mM NH₄HCO₃) |
| HPLC-13 | XBridge C18 OBD, 19 × 250 mm, 5 mm; MeCN/H₂O (+10 mM NH₄HCO₃) |
| HPLC-14 | XBridge C18 OBD, 25 × 150 mm, 5 mm; MeCN/H₂O (+0.04% NH₄OH + |
| | 10 mM NH₄HCO₃) |
| HPLC-15 | XBridge C18 OBD, 25 × 150 mm, 5 mm; MeCN/H₂O (+0 mM NH₄HCO₃) |
| HPLC-16 | XBridge C18 OBD, 30 × 150 mm, 5 mm; MeCN/H₂O (+0.05% HCO₂H) |
| HPLC-17 | XBridge C18 OBD, 30 × 150 mm, 5 mm; MeCN/H₂O (+0.05% NH₄HCO₃) |
| HPLC-18 | XBridge C18 OBD, 30 × 150 mm, 5 mm; MeCN/H₂O (+0.05% NH₄OH) |
| HPLC-19 | XBridge C18 OBD, 30 × 150 mm, 5 mm; MeCN/H₂O (+0.05% TFA) |
| HPLC-20 | XBridge C18 OBD, 30 × 150 mm, 5 mm; MeCN/H₂O (+0.1% HCO₂H) |
| HPLC-21 | XBridge C18 OBD, 30 × 150 mm, 5 mm; MeCN/H₂O (+0.1% NH₄OH + |
| | 10 mM NH₄HCO₃) |
| HPLC-22 | XBridge C18 OBD, 30 × 150 mm, 5 mm; MeCN/H₂O (+0.5% TFA) |
| HPLC-23 | XBridge C18 OBD, 30 × 150 mm, 5 mm; MeCN/H₂O (+10 mM NH₄HCO₃) |
| HPLC-24 | XBridge C18 OBD, 30 × 150 mm, 5 mm; MeCN/H₂O (+1-0 mM NH₄OH) |
| HPLC-25 | XBridge C18 OBD, 30 × 150 mm, 10 mm; MeCN/H₂O (10 mM NH₄HCO₃) |
| HPLC-26 | XBridge C18 OBD, 50 × 150 mm, 10 mm; MeCN/H₂O (10 mM NH₄HCO₃) |
| HPLC-27 | Xselect CSH OBD, 30 × 150 mm, 5 mm, MeCN/H₂O (+0.1% HCO₂H) |
| HPLC-28 | Xselect CSH OBD, 30 × 150 mm, 5 mm, MeCN/H₂O (+10 mM NH₄HCO₃) |
| HPLC-29 | Kromasil C18, 100 × 30 mm, 5um, MeCN/H₂O (+0.225% HCO₂H) |
| HPLC-30 | YMC-Actus Triart C18, MeCN/H₂O (+10 mM NH₄HCO₃) |

### General Synthesis Schemes

According to a first process, compounds of Formula (I) may be prepared from the compounds of Formulae (II) and (III), as illustrated by Scheme 1.

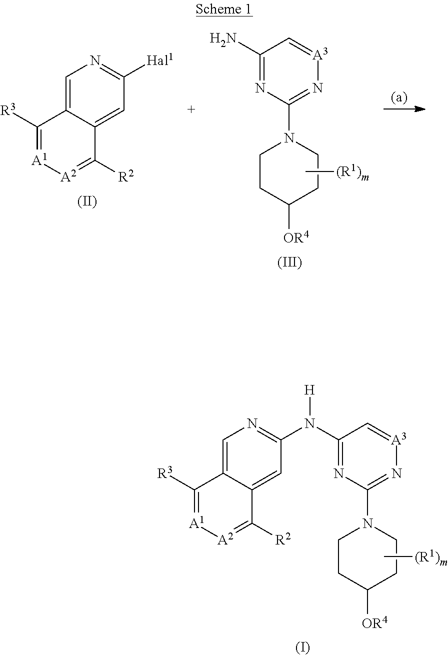

Hal¹ is a halogen, preferably Cl

The compound of Formula (I) may be prepared according to process step (a), a Buchwald-Hartwig cross coupling reaction. Typical conditions comprise, reaction of the amine of Formula (III) with the halide of Formula (II) in the presence of a suitable inorganic base, a suitable palladium catalyst in a suitable solvent at elevated temperature. Preferred conditions comprise, reaction of the compounds of Formulae (II) and (III) in the presence of, BINAP Pd G3, RuPhos Pd G3, BrettPhos Pd G3, BrettPhos Pd G4, XPhos Pd G4, Xantphos Pd G2 or Pd(allyl)(Brett)]Otf, or CPhos, Xantphos, BrettPhos or BINAP in combination with Pd₂(dba)₃ or Xantphos Pd G2, in the presence of a suitable base such as Cs₂CO₃ or K₂CO₃, in a suitable solvent such as dioxane, toluene or DMF, at between 90° C. and 130° C.

According to a second process, compounds of Formula (II) may be prepared from compounds of Formula (IV) as illustrated by Scheme 2.

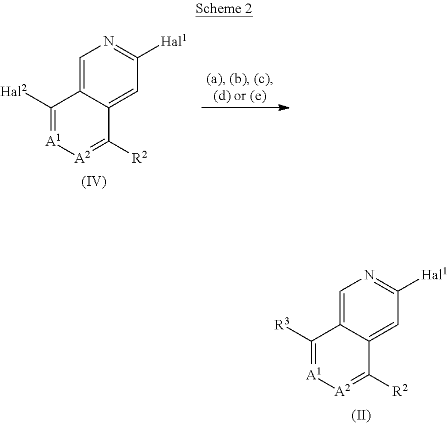

Hal² is halogen, preferably Br or Cl or I.

Wherein R³ is N-linked, the compound of Formula (II) may be prepared from the halide of Formula (IV) and R³NH according to process step (a) a Buchwald-Hartwig cross coupling reaction, as previously described in Scheme 1.

Alternatively, compounds of Formula (II), may be prepared from the halide of Formula (IV) and R³NH according to process step (b) an amination reaction. Typical conditions comprise, reaction of the amine, R³NH, with the halide of Formula (IV) in the presence of a suitable organic base, such as TEA or DIPEA in a suitable solvent such as DMSO, DMF, butan-2-ol or IPA at elevated temperature, such as 100° C., optionally under microwave irradiation.

Alternatively, compounds of Formula (II), may be prepared from the halide of Formula (IV) and R³NH, according to process step (c) a mild Ullman type reaction as described in Org Lett 2003, 5, 14, 2453-2455. Typical conditions comprise, reaction of the amine, R³NH, with the halide of Formula (IV) in the presence of K₃PO₄, L-proline and CuI in DMSO at between 80 and 100° C.

Wherein R³ is C-linked, the compound of Formula (II) may be prepared from the compound of Formula (IV) by process step (d) a metal catalysed cross-coupling reaction with a suitable R³ containing nuceophile. Typical cross-coupling conditions comprise a palladium catalyst containing suitable phosphine ligands, such as Pd(amphos)Cl₂, Pd(dtbpf)Cl₂, Pd(dppf)Cl₂, Pd(OAc)₂ and cataCXium® A, in the presence of a zinc or boron nucleophile, optionally in the presence of an inorganic or organic base, such as Na₂CO₃, K₂CO₃ or K₃PO₄, in a suitable solvent, such as DMA, DME, dioxane, aqueous dioxane or DMF at between rt and elevated temperature.

Alternatively, wherein R³ is a C-linked heteroaryl group, the compound of Formula (II) may be prepared from the compound of Formula (IV) and R³H, by process step (e) a copper catalysed coupling reaction. Typical conditions comprise reaction of the compound of Formula (IV) with R₃H, in the presence of a strong base such as tBuOLi in the presence of catalytic CuI/1,10-phenanthroline in a suitable solvent such as DMF at elevated temperature.

According to a third process, compounds of Formula (IV) may be prepared from compounds of Formula (V), (VI) and (VII) as illustrated by Scheme 3.

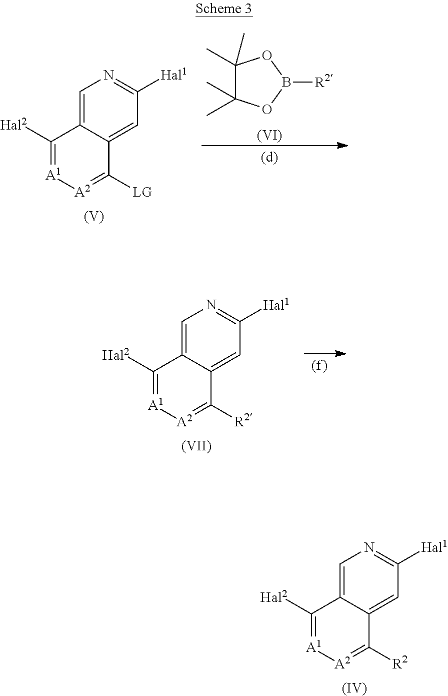

LG is a leaving group, typically a halogen or triflate and preferably Br, I or triflate.

R^{2′} is an unsaturated analogue of R²,

The compound of Formula (VII) may be prepared from the compound of Formula (V) and the boronate ester, of Formula (VI), according to process step (d) an organometallic catalysed cross-coupling reaction as previously described in Scheme 2.

The compound of Formula (IV) may be prepared from the compound of Formula (VII) by process step (f) a hydrogenation reaction in the presence of a suitable catalyst such as Pd/C or PtO₂ in a suitable solvent, such as EtOAc under an atmosphere of H₂ at about rt.

According to a fourth process, compounds of Formula (II)(A), wherein R³ is N-linked, may be prepared from the compounds of Formulae (VI), (VIII), (IX) and (X) as illustrated in Scheme 4.

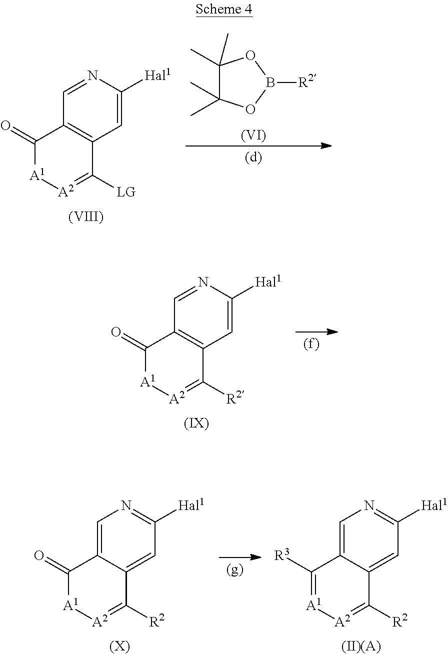

The compound of Formula (IX) may be prepared from the compound of Formula (VIII) and the boronate ester, of Formula (VI), according to process step (d) as described in Scheme 2.

The compound of Formula (X) may be prepared from the compound of Formula (IX) by process step (f) a hydrogenation reaction, as described in Scheme 3.

The compound of Formula (II) may be prepared from the compound of Formula (X) and the amine, R³NH, according to process step (g). Typical conditions comprise reaction of the compound of Formula (X) with the amine, R³NH, in the presence of a suitable coupling reagent such as BOP or HBTU, in the presence of a suitable non-nucleophilic base, such as DBU in a suitable solvent, such as DMF at rt.

According to a fifth process, compounds of Formula (II) may be prepared from compounds of Formula (IV) and (XI) as illustrated in Scheme 5.

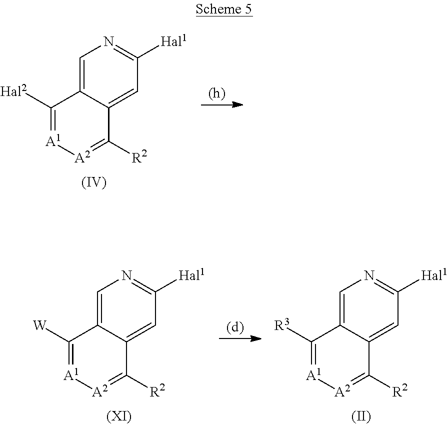

W is a boronic acid or suitable boronate ester, such as boronic acid pinacol ester.

The compound of Formula (XI) may be prepared from the compound of Formula (IV), according to process step (h), a boronate ester formation achieved by treatment with a suitable boronate such as (BPin)₂, in the presence of a suitable inorganic base, such as K₂CO₃ or KOAc and a suitable catalyst, such as, Pd(dppf)Cl₂ in a suitable non-polar solvent at between rt and elevated temperature. Preferred conditions comprise, treatment of the compound of Formula (IV) with (BPin)₂ in the presence of Pd(dppf)Cl₂ in the presence of KOAc in dioxane, at 90° C.

The compound of Formula (II) may be prepared from the compound of Formula (XI) and R³H, according to step (d), as described in Scheme 2.

According to a sixth process, wherein, R² is directly linked C₁-C₄ alkoxy, compounds of Formula (II)(B) may be prepared from compounds of Formula (V), (XII), (XIII), (XIV), (XV), (X)(B) and (IV)(B) as illustrated in Scheme 6.

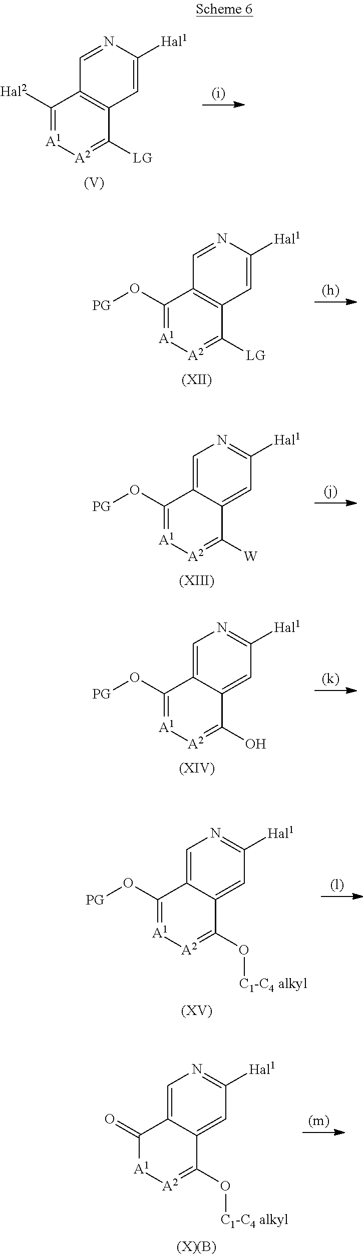

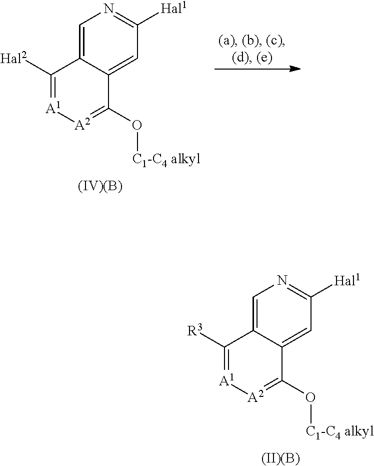

PG is a suitable OH protecting group, preferably a benzyl group.

The compound of Formula (XII) may be prepared from the compound of Formula (V) according to process step (i). Typical conditions comprise reaction of the halide of Formula (V) with a protected alcohol, PGOH, in the presence of a strong base, such as NaH in a suitable solvent such as DMF, at rt.

The compound of Formula (XIII) may be prepared from the compound of Formula (XII) by process step (h) as previously described in Scheme 5.

The compound of Formula (XIV) may be prepared from the compound of Formula (XIII) according to process step (j), an oxidation of the boronate ester. Typical conditions, comprise reaction of the compound of Formula (XIII) with Oxone® in a suitable solvent such as acetone at between 0° C. and rt, optionally in the presence of NaHCO₃.

The compounds of Formula (XV) may be prepared from the compound of Formula (XIV) by process step (k) an alkylation reaction. Typical conditions comprise reaction of the alcohol of Formula (XIV) with an alkylating agent, C₁-C₄alkyl-LG, wherein LG is as previously defined, in the presence of a suitable inorganic base, such as K₂CO₃ or NaH, in a suitable solvent such as dioxane or DMF at rt.

The compound of Formula (X)(B) may be prepared from the compound of Formula (XV) by process step (l), a de-protection reaction performed under standard conditions, such as treatment of the compound of Formula (XV) with TFA in DCM at rt.

The compound of Formula (IV)(B) may be prepared from the compound of Formula (X)(B) by process step (m) a halogenation reaction, typically a chlorination reaction. Preferred conditions comprise reaction of the compound of Formula (X)(B) with a chlorinating agent such as POCl₃ at elevated temperature, such as 90° C.

The compound of Formula (II)(B) may be prepared from the compound of Formula (IV)(B) according to process steps (a), (b), (c), (d) or (e) as previously described in Scheme 2.

According to a seventh process, compounds of Formula (I) may be prepared from compounds of Formulae (III), (IV), (XVI) and (XVII) as illustrated in Scheme 7.

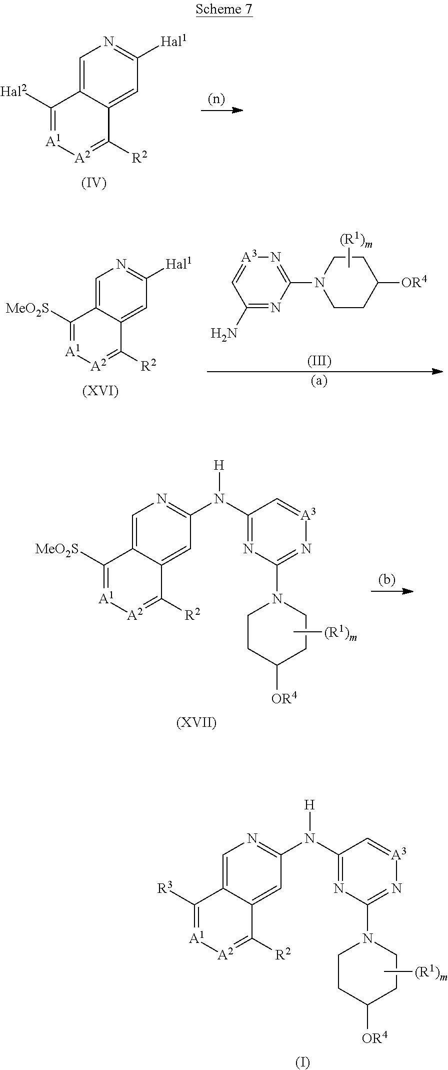

The compound of Formula (XVI) may be prepared from the compound of Formula (IV), according to process step (n). Typical conditions comprise reaction of the halide of Formula (IV) with sodium methanesulfinate in a suitable solvent such as DMA at rt.

The compound of Formula (XVII) may be prepared from the compounds of Formulae (XVII) and (III), by process step (a), as previously described in Scheme 1.

The compound of Formula (I) may be prepared from the compound of Formula (XVII) by process step (b) as described in Scheme 2.

According to an eighth process, the compound of Formula (III) may be prepared from the compounds of Formulae (XVIII) and (XIX) as illustrated in Scheme 8.

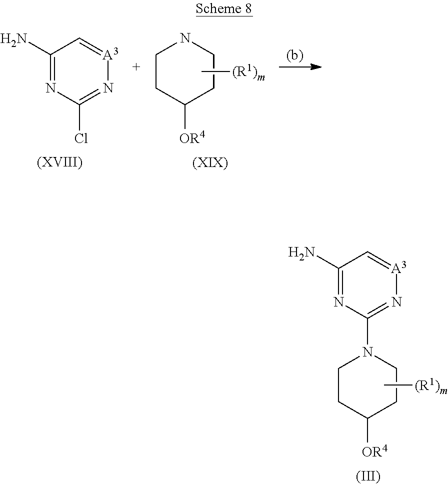

The compound of Formula (III) may be prepared from the chloride of Formula (XVIII) and the amine of Formula (XIX), according to process step (b) an amination reaction, as previously described in Scheme 2.

The compounds of Formulae (IV), (V), (VI), (VIII), (XVIII) and (XIX) are either commercially available or may be prepared by analogy to methods known in the literature, or the methods described in the Experimental section below.

Compounds of Formula (I), (II) and (IV) may be converted to alternative compounds of Formula (I), (II) and (IV), by standard chemical transformations, known to those skilled in the art. Examples of these transformations include, but are not limited to:
reduction of an ester to an alcohol using LiAlH₄, reduction of a ketone to a secondary alcohol using NaBH₄, hydrolysis of an ester to an acid using aqueous alkali metal hydroxide, Grignard reaction of an ester to provide a tertiary alcohol, alkylation or acetylation of a heteroatom, such as N or O, reductive amination of a N atom, dehydration of an amide to provide a nitrile group, coupling of carboxylic acids and amines in the presence of a suitable coupling agent, to provide an amide, halogenation, such as fluorination using a fluorinating agent, such as DAST, or iodination using PPh₃, imidazole and iodine, transformation of aryl iodides to ketones using Stille methodology, or to aryl ketones via a palladium catalysed carbonylation reaction, and the synthesis of heterocycles, such as 1,2,4-oxadiazoles as described in Comprehensive Heterocyclic Synthesis.

It will be appreciated by those skilled in the art that it may be necessary to utilise a suitable protecting group strategy for the preparation of compounds of Formula (I). Typical protecting groups may comprise, carbamate and preferably Boc for the protection of amines, a TBS or benzyl group for the protection of a primary alcohol, or a benzyl group for the protection of a phenolic OH.

It will be further appreciated that it may be necessary or desirable to carry out the transformations in a different order from that described in the schemes, or to modify one or more of the transformations, to provide the desired compound of the invention.

### Synthesis of Intermediates

### Preparation 1

### Tert-butyl 5-methyl-4-(trimethylsilyloxy)-5,6-dihydropyridine-1(2H)-carboxylate

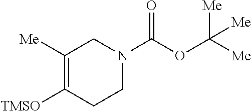

Trimethylsilyl trifluoromethanesulfonate (12.50 g, 56.25 mmol) was added drop wise to a pre-cooled solution of tert-butyl 3-methyl-4-oxopiperidine-1-carboxylate (10 g, 46.88 mmol) and TEA (11.38 g, 112.5 mmol) in toluene (100 mL) at 0° C. and the resulting mixture stirred for 4 h at 0° C. The solution was quenched with water (50 mL) and extracted with EtOAc (×2). The combined organics were washed (brine), dried (Na₂SO₄) and evaporated to dryness in vacuo to afford the title compound as a yellow oil (10.5 g, 78.5%). ¹H NMR (400 MHz, DMSO-d₆) δ: 3.68-3.66 (m, 2H), 3.43 (t, 2H), 2.05 (tq, 2H), 1.53-1.47 (m, 3H), 1.41 (s, 9H), 0.15 (s, 9H).

### Preparation 2

### tert-butyl 3-fluoro-3-methyl-4-oxopiperidine-1-carboxylate

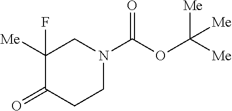 [SMILES: CC(C)(C)OC(=O)N1CCC(=O)C(C)(F)C1]

A mixture of tert-butyl 5-methyl-4-(trimethylsilyloxy)-5,6-dihydropyridine-1(2H)-carboxylate (Preparation 1, 10 g, 35.0 mmol) and SelectFluor (13.6 g, 38.5 mmol) in MeCN (100 mL) was stirred for 1 h at 0° C. The solution was diluted with water (100 mL) and extracted with EtOAc. The combined organics were washed (brine), dried (Na₂SO₄) and evaporated to dryness in vacuo to afford the title compound as a pale, yellow oil (8 g, 98.8%).

### Preparation 3

### tert-butyl 3-fluoro-4-hydroxy-3-methylpiperidine-1-carboxylate

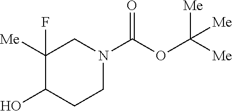 [SMILES: CC(C)(C)OC(=O)N1CCC(O)C(C)(F)C1]

A mixture of tert-butyl 3-fluoro-3-methyl-4-oxopiperidine-1-carboxylate (Preparation 2, 7 g, 30.2 mmol) and NaBH₄ (1.37 g, 36.2 mmol) in MeOH (70 mL) was stirred for 3 h at rt. The reaction mixture was extracted with EtOAc and the combined organics were washed (brine), dried (Na₂SO₄) and evaporated to dryness in vacuo to give the title compound as a light-yellow oil (7 g, 99%).

### Preparation 4

### 3-fluoro-3-methylpiperidin-4-ol hydrochloride

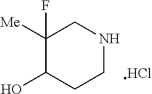

Hydrochloric acid (4 M in dioxane, 50 mL) was added to a solution of tert-butyl 3-fluoro-4-hydroxy-3-methylpiperidine-1-carboxylate (Preparation 3, 7 g, 30.0 mmol) in DCM (70 mL) and the resulting mixture stirred at rt for 3 h. The reaction precipitate was collected by filtration to afford the title compound as a white solid (4.5 g).

### Preparation 5

### 1-(4-aminopyrimidin-2-yl)-3-fluoro-3-methylpiperidin-4-ol

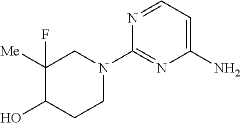 [SMILES: CC1(F)CN(c2nccc(N)n2)CCC1O]

A mixture of 2-chloropyrimidin-4-amine (2.7 g, 20.8 mmol), 3-fluoro-3-methylpiperidin-4-ol hydrochloride (Preparation 4, 3.86 g, 22.8 mmol) and TEA (6.30 g, 62.4 mmol) in IPA (45 mL) was stirred for 5 h at 130° C. in a sealed vial. The reaction mixture was cooled to rt and the solids removed by filtration. The filtrate was evaporated to dryness in vacuo to give the title compound as a yellow oil (6 g) which was used without further purification.

### Preparations 6 and 7

### Cis-rac-1-(4-aminopyrimidin-2-yl)-3-fluoro-3-methylpiperidin-4-ol and trans-rac-1-(4-aminopyrimidin-2-yl)-3-fluoro-3-methylpiperidin-4-ol

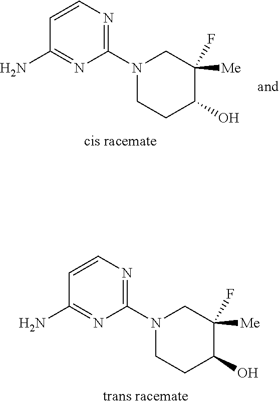 [SMILES: C[C@]1(F)CN(c2nccc(N)n2)CC[C@H]1O ; C[C@]1(F)CN(c2nccc(N)n2)CC[C@@H]1O]

1-(4-Aminopyrimidin-2-yl)-3-fluoro-3-methylpiperidin-4-ol (Preparation 5) was purified by HPLC-17 to afford the title compounds. Peak 1; Preparation 6, cis-rac-1-(4-aminopyrimidin-2-yl)-3-fluoro-3-methylpiperidin-4-ol as a white solid (1.3 g, 26.1%) and Peak 2; Preparation 7, trans-rac-1-(4-aminopyrimidin-2-yl)-3-fluoro-3-methylpiperidin-4-ol as a white solid (500 mg, 10%).

### Preparation 8 and 9

### (3S,4R)-1-(4-aminopyrimidin-2-yl)-3-fluoro-3-methylpiperidin-4-ol and (3R,4S)-1-(4-aminopyrimidin-2-yl)-3-fluoro-3-methylpiperidin-4-ol

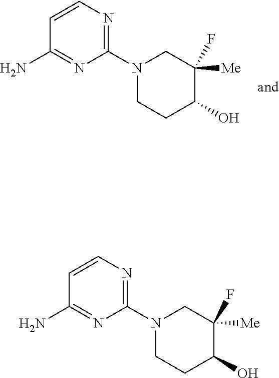 [SMILES: C[C@]1(F)CN(c2nccc(N)n2)CC[C@H]1O ; C[C@@]1(F)CN(c2nccc(N)n2)CC[C@@H]1O]

Cis-rac-1-(4-aminopyrimidin-2-yl)-3-fluoro-3-methylpiperidin-4-ol (Preparation 6) was separated by prep-SFC (Phenomenex Lux 5p Cellulose-3, 5×25 cm, 5 mm; 50% MeOH (+0.1% DEA) in CO₂) to afford the title enantiomers.

Peak 1, Preparation 8: (3S,4R)-1-(4-aminopyrimidin-2-yl)-3-fluoro-3-methylpiperidin-4-ol (Stereochemistry assigned by x-ray crystallography of a related compound (not shown)) as a white solid (500 mg) and

Peak 2, Preparation 9: (3R,4S)-1-(4-aminopyrimidin-2-yl)-3-fluoro-3-methylpiperidin-4-ol (500 mg).

LCMS m/z=227 [M+H]+; ¹H NMR (400 MHz, DMSO-d₆) δ: 7.71 (d, 1H), 6.37 (s, 2H), 5.69 (d, 1H), 4.93 (d, 1H), 4.66 (ddd, 1H), 4.60-4.50 (m, 1H), 3.44 (ddt, 1H), 3.02-2.78 (m, 2H), 1.69-1.53 (m, 2H), 1.31 (d, 3H).

### Preparation 10

### Cis-rac-tert-butyl 3-fluoro-4-hydroxy-4-methylpiperidine-1-carboxylate

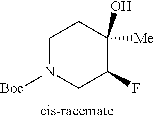 [SMILES: CC(C)(C)OC(=O)N1CC[C@@](C)(O)[C@@H](F)C1]

MeMgBr (9.2 mL, 27.6 mmol) was added to a solution of tert-butyl 3-fluoro-4-oxopiperidine-1-carboxylate (5 g, 2.3 mmol) in THF (50 mL) at −78° C. The mixture was stirred overnight at rt. The reaction mixture was carefully diluted with sat. NH₄Cl (aq), extracted with EtOAc and washed with brine. The combined organics were dried (Na₂SO₄) and evaporated, to afford the title compound as a yellow solid (4.8 g, 90%). LCMS m/z=178 [M+H−56]⁺.

### Preparation 11

### Cis-rac-3-fluoro-4-methylpiperidin-4-ol hydrochloride

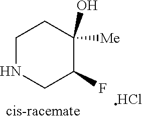

Cis-rac-tert-butyl 3-fluoro-4-hydroxy-4-methylpiperidine-1-carboxylate (Preparation 10, 4.8 g, 20 mmol) in HCl/dioxane (50 mL) was stirred at rt for 4 h. The reaction mixture was evaporated to afford the title compound as a yellow solid (3 g, crude) which was used directly for the next step. LCMS m/z=134 [M+H]⁺.

### Preparation 12

### cis-rac-1-(4-aminopyrimidin-2-yl)-3-fluoro-4-methylpiperidin-4-ol

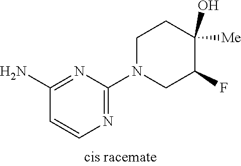 [SMILES: C[C@@]1(O)CCN(c2nccc(N)n2)C[C@@H]1F]

A mixture of 2-chloropyrimidin-4-amine (1.5 g, 11.5 mmol), cis-rac-3-fluoro-4-methylpiperidin-4-ol hydrochloride (Preparation 11, 3 g) and DIPEA (11.9 g, 92.3 mmol) in DMSO (40 mL) was stirred overnight at 120° C. The reaction mixture was diluted with water, extracted (EtOAc) and washed with brine. The combined organics were dried (Na₂SO₄) and evaporated to dryness in vacuo to afford the title compound as a light-yellow solid (1.3 g). LCMS m/z=227 [M+H]+.

### Preparation 13 and 14

### (3S,4R)-1-(4-aminopyrimidin-2-yl)-3-fluoro-4-methylpiperidin-4-ol and (3R,4S)-1-(4-aminopyrimidin-2-yl)-3-fluoro-4-methylpiperidin-4-ol

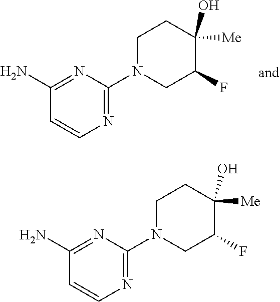 [SMILES: C[C@@]1(O)CCN(c2nccc(N)n2)C[C@@H]1F ; C[C@]1(O)CCN(c2nccc(N)n2)C[C@H]1F]

Cis-rac-1-(4-aminopyrimidin-2-yl)-3-fluoro-4-methylpiperidin-4-ol (Preparation 12) was separated by preparative SFC using a CHIRAL Cellulose-SJ (4.6×150 mm, 5 μm) column, eluting with CO₂/MeOH (0.1% DEA) at a flow rate of 4 g/min, to afford Peak 1, Preparation 13: (3S,4R)-1-(4-aminopyrimidin-2-yl)-3-fluoro-4-methylpiperidin-4-ol (450 mg, Stereochemistry assigned by x-ray crystallography of a related compound (not shown)) as a white solid and Peak 2, Preparation 14: (3R,4S)-1-(4-aminopyrimidin-2-yl)-3-fluoro-4-methylpiperidin-4-ol (470 mg) as a white solid. Peak 1, Preparation 13: (3S,4R)-1-(4-aminopyrimidin-2-yl)-3-fluoro-4-methylpiperidin-4-ol. ¹H NMR (300 MHz, DMSO-d₆) δ: 7.73 (d, 1H), 6.40 (s, 2H), 5.72 (d, 1H), 4.71 (s, 1H), 4.39-3.92 (m, 3H), 3.38 (dddd, 2H), 1.62 (q, 1H), 1.42 (td, 1H), 1.20 (s, 3H). Peak 2, Preparation 14: (3R,4S)-1-(4-aminopyrimidin-2-yl)-3-fluoro-4-methylpiperidin-4-ol. ¹H NMR (300 MHz, DMSO-d₆) δ: 7.73 (d, 1H), 6.40 (s, 2H), 5.72 (d, 1H), 4.71 (s, 1H), 4.36-4.07 (m, 2H), 4.07-3.95 (m, 1H), 3.44 (ddd, 1H), 3.31 (ddd, 1H), 1.61 (ddt, 1H), 1.41 (ddd, 1H), 1.20 (s, 3H).

### Preparation 15

### Cis-rac-1-(4-aminopyrimidin-2-yl)-4-methoxypiperidin-3-ol

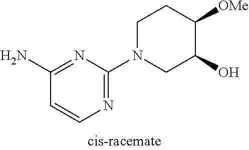 [SMILES: CO[C@@H]1CCN(c2nccc(N)n2)C[C@@H]1O]

A mixture of cis-rac-4-methoxypiperidin-3-ol (1.7 g, 13 mmol), 2-chloropyrimidin-4-amine (1.7 g, 13 mmol) and TEA (2.6 g, 26 mmol) in IPA (15 mL) was stirred overnight at 100° C. The mixture was concentrated in vacuo and the residue purified by column chromatography (5% MeOH in DCM) to afford the title compound as a yellow solid (2.4 g, 82.7%). LCMS m/z=225 [M+H]+.

### Preparation 16 and 17

### (3S,4R)-1-(4-aminopyrimidin-2-yl)-4-methoxypiperidin-3-ol and (3R,4S)-1-(4-aminopyrimidin-2-yl)-4-methoxypiperidin-3-ol

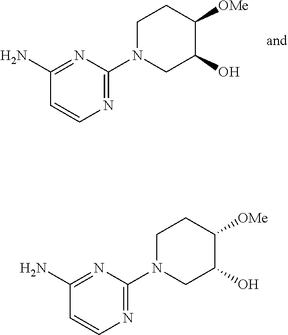 [SMILES: CO[C@@H]1CCN(c2nccc(N)n2)C[C@@H]1O ; CO[C@H]1CCN(c2nccc(N)n2)C[C@H]1O]

Cis-rac-1-(4-aminopyrimidin-2-yl)-4-methoxypiperidin-3-ol (Preparation 15, 2.4 g) was separated by Chiral-SFC using a Chiralpak IA, 4.6×150 mm, 5 mm column, eluting with 10% MeOH (+0.1% DEA) in CO₂) to afford the title compounds.

Peak 1: (3S,4R)-1-(4-aminopyrimidin-2-yl)-4-methoxypiperidin-3-ol or (3R,4S)-1-(4-aminopyrimidin-2-yl)-4-methoxypiperidin-3-ol (900 mg) and

Peak 2: (3R,4S)-1-(4-aminopyrimidin-2-yl)-4-methoxypiperidin-3-ol or (3S,4R)-1-(4-aminopyrimidin-2-yl)-4-methoxypiperidin-3-ol (890 mg).

### Preparation 18 and 19

### (3S,4S)-1-(4-aminopyrimidin-2-yl)-4-methoxypiperidin-3-ol and (3R,4R)-1-(4-aminopyrimidin-2-yl)-4-methoxypiperidin-3-ol

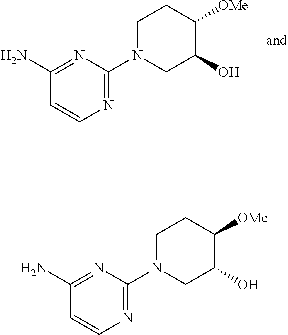 [SMILES: CO[C@H]1CCN(c2nccc(N)n2)C[C@@H]1O ; CO[C@@H]1CCN(c2nccc(N)n2)C[C@H]1O]

Part 1: 2-Chloropyrimidin-4-amine (987 mg, 7.62 mmol) was added to trans-rac-4-methoxypiperidin-3-ol (1.0 g, 7.62 mmol) and TEA (2.30 g, 22.8 mmol) in IPA (20 mL) at rt and the mixture stirred at 100° C. for 16 h. The mixture was concentrated in vacuo and the residue purified by a silica gel column (20:1 DCM/MeOH) to afford trans-rac-1-(4-aminopyrimidin-2-yl)-4-methoxypiperidin-3-ol as a colorless oil (1.2 g).

Part 2: Trans-rac-1-(4-aminopyrimidin-2-yl)-4-methoxypiperidin-3-ol (Part 1, 1.2 g, 5.35 mmol) was purified by chiral-SFC (CHIRALPAK IC, 20×250 mm, 5 mm; 25% EtOH (8 mM NH₃·MeOH) in CO₂) to afford the title compounds.

Peak 1; (3S,4S)-1-(4-aminopyrimidin-2-yl)-4-methoxypiperidin-3-ol or (3R,4R)-1-(4-aminopyrimidin-2-yl)-4-methoxypiperidin-3-ol as a white solid (450 mg) and

Peak 2; (3R,4R)-1-(4-aminopyrimidin-2-yl)-4-methoxypiperidin-3-ol or (3S,4S)-1-(4-aminopyrimidin-2-yl)-4-methoxypiperidin-3-ol as a white solid (460 mg). LCMS m/z=225 [M+H]⁺.

### Preparation 20

### Cis-rac-1-(4-aminopyrimidin-2-yl)-5-fluoro-3,3-dimethylpiperidin-4-ol

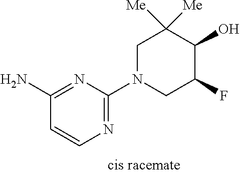 [SMILES: CC1(C)CN(c2nccc(N)n2)C[C@H](F)[C@@H]1O]

Part 1: Cis-rac-tert-butyl 5-fluoro-4-hydroxy-3,3-dimethylpiperidine-1-carboxylate (4.7 g, 19.0 mmol) was added to a solution of HCl in dioxane (30 mL) and the resulting mixture stirred at rt for 16 h. The solvent was removed under reduced pressure to afford cis-rac-5-fluoro-3,3-dimethylpiperidin-4-ol hydrochloride as a white solid (3.6 g) which was used without further purification in Part 2.

Part 2: TEA (3.83 g, 38.0 mmol) was added to a mixture of cis-rac-5-fluoro-3,3-dimethylpiperidin-4-ol hydrochloride (Part 1, 3.6 g, 19.0 mmol) and 2-chloropyrimidin-4-amine (2.46 g, 19.0 mmol) in IPA (10 mL) and the resulting mixture stirred at 100° C. for 3 h. The solids were removed by filtration and the filtrate was concentrated under reduced pressure. The residue was purified by HPLC-21, to afford the title compound as a white solid (1.8 g, 39.4%). LCMS m/z=241 [M+H]⁺.

### Preparation 21 and 22

### (4R,5S)-1-(4-aminopyrimidin-2-yl)-5-fluoro-3,3-dimethylpiperidin-4-ol and (4S,5R)-1-(4-aminopyrimidin-2-yl)-5-fluoro-3,3-dimethylpiperidin-4-ol

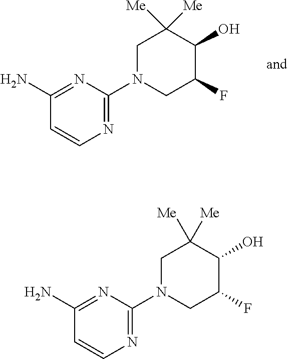 [SMILES: CC1(C)CN(c2nccc(N)n2)C[C@H](F)[C@@H]1O ; CC1(C)CN(c2nccc(N)n2)C[C@@H](F)[C@H]1O]

Cis-rac-1-(4-aminopyrimidin-2-yl)-5-fluoro-3,3-dimethylpiperidin-4-ol (Preparation 20, 1.8 g) was separated by preparative chiral-SFC (EnantioPak-A1-5(02), 50×250 mm, 5 mm; 60% EtOH (0.1% DEA) in CO₂) to afford the title compounds. Peak 1: (4R,5S)-1-(4-aminopyrimidin-2-yl)-5-fluoro-3,3-dimethylpiperidin-4-ol or (4S,5R)-1-(4-aminopyrimidin-2-yl)-5-fluoro-3,3-dimethylpiperidin-4-ol (776 mg, 43.3%) as a white solid and

Peak 2: (4S,5R)-1-(4-aminopyrimidin-2-yl)-5-fluoro-3,3-dimethylpiperidin-4-ol or (4R,5S)-1-(4-aminopyrimidin-2-yl)-5-fluoro-3,3-dimethylpiperidin-4-ol (700 mg, 39.1%) as a white solid. LCMS m/z=241 [M+H]+

### Preparation 23

### 1-(4-aminopyrimidin-2-yl)-4-methylpiperidin-4-ol

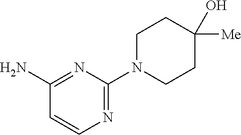 [SMILES: CC1(O)CCN(c2nccc(N)n2)CC1]

A mixture of 4-methylpiperidin-4-ol (230 mg, 2 mmol), 2-chloropyrimidin-4-amine (258 mg, 2 mmol) and TEA (300 mg, 3 mmol) in IPA (5 mL) was stirred overnight at rt. The solvent was evaporated to dryness in vacuo and the residue purified by prep-TLC (6% MeOH in DCM) to afford the title compound (210 mg, 50%). LCMS m/z=209 [M+H]⁺.

### Preparation 24

### tert-butyl (3S,4R)-4-(2-((tert-butyldimethylsilyl)oxy)ethoxy)-3-fluoropiperidine-1-carboxylate

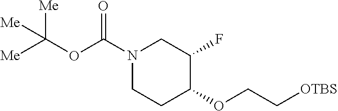 [SMILES: CC(C)(C)OC(=O)N1CC[C@@H](OCCO[Si](C)(C)C(C)(C)C)[C@@H](F)C1]

NaH (1.35 g, 33.9 mmol) was added batchwise to tert-butyl (3S,4R)-3-fluoro-4-hydroxypiperidine-1-carboxylate (3.0 g, 13.6 mmol) in DMF (10 mL) at 0° C. and the solution stirred at 0° C. for 20 min. (2-Bromoethoxy)(tert-butyl)dimethylsilane (9.76 g, 40.8 mmol) was added and the mixture stirred at rt for 16 h. The reaction mixture was diluted with EtOAc, washed with brine, dried (Na₂SO₄) and evaporated to dryness in vacuo. The residue was purified by column chromatography (10:1 PE/EtOAc) to afford the title compound as a colorless oil (3 g, 58.4%).

### Preparation 25

### 2-(((3S,4R)-1-(4-aminopyrimidin-2-yl)-3-fluoropiperidin-4-yl)oxy)ethan-1-ol

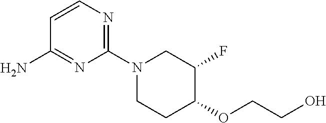 [SMILES: Nc1ccnc(N2CC[C@@H](OCCO)[C@@H](F)C2)n1]

TFA (15 mL) was added to tert-butyl (3S,4R)-4-(2-((tert-butyldimethylsilyl)oxy)ethoxy)-3-fluoropiperidine-1-carboxylate (Preparation 24, 3.0 g, 7.94 mmol) in DCM (20 mL) at rt and the resulting mixture stirred at rt for 1 h. The mixture was concentrated in vacuo, 2-chloropyrimidin-4-amine (873 mg, 6.74 mmol) and DIPEA (629 mg, 4.88 mmol) in DMSO (10 mL) added and the mixture stirred overnight at 100° C. The reaction was diluted with EtOAc (50 mL) and washed with brine. The organic solution was dried (Na₂SO₄) and evaporated to dryness in vacuo and the residue purified by column chromatography (1:15 MeOH/EtOAc) to afford the title compound as a yellow solid (1.1 g, 56%). LCMS m/z=257 [M+H]⁺. ¹H NMR (400 MHz, DMSO-d₆) δ: 7.72 (d, 1H), 6.41 (s, 2H), 5.71 (d, 1H), 4.94-4.69 (m, 1H), 4.67-4.52 (m, 2H), 4.34 (d, 1H), 3.72-3.45 (m, 5H), 3.31-3.19 (m, 1H), 3.07 (t, 1H), 1.77-1.44 (m, 2H).

### Preparation 26

### tert-butyl (3R,4S)-4-(2-((tert-butyldimethylsilyl)oxy)ethoxy)-3-fluoropiperidine-1-carboxylate

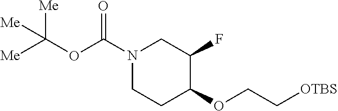 [SMILES: CC(C)(C)OC(=O)N1CC[C@H](OCCO[Si](C)(C)C(C)(C)C)[C@H](F)C1]

The title compound was obtained as a colorless oil, 1.1 g, 63.8%, from tert-butyl (3R,4S)-3-fluoro-4-hydroxypiperidine-1-carboxylate and (2-bromoethoxy)(tert-butyl)dimethylsilane (3.25 g, 13.6 mmol) following the procedure described in Preparation 24.

### Preparation 27

### 2-(((3R,4S)-1-(4-aminopyrimidin-2-yl)-3-fluoropiperidin-4-yl)oxy)ethan-1-ol

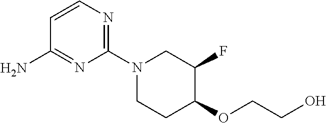 [SMILES: Nc1ccnc(N2CC[C@H](OCCO)[C@H](F)C2)n1]

The title compound was obtained as a yellow solid, 450 mg, from tert-butyl (3R,4S)-4-(2-((tert-butyldimethylsilyl)oxy)ethoxy)-3-fluoropiperidine-1-carboxylate (Preparation 26), following the procedure described in Preparation 25. LCMS m/z=257 [M+H]⁺; 1H-NMR (400 MHz, DMSO-d₆) δ 7.72 (d, 1H), 6.41 (s, 2H), 5.71 (d, 1H), 4.91-4.73 (m, 1H), 4.67-4.50 (m, 2H), 4.34 (d, 1H), 3.69-3.46 (m, 5H), 3.30-3.17 (m, 1H), 3.06 (d, 1H), 1.80-1.54 (m, 2H).

### Preparation 28

### tert-butyl (3R,4S)-3-fluoro-4-(methoxy-d3)piperidine-1-carboxylate

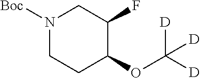 [SMILES: [2H]C([2H])([2H])O[C@H]1CCN(C(=O)OC(C)(C)C)C[C@H]1F]

NaH (218 mg, 9.08 mmol) was added to tert-butyl (3R,4S)-3-fluoro-4-hydroxypiperidine-1-carboxylate (1.0 g, 4.56 mmol) in DMF (20 mL) at 0° C. and stirred for 20 mins before CD₃I (3.30 g, 22.8 mmol) was added and the solution stirred at rt for 16 h. The reaction was quenched by the addition of H₂O (5 mL) and the solids removed by filtration. The filtrate was extracted with EtOAc and the combined organics washed with brine and evaporated to dryness in vacuo to afford the title compound as a light-yellow oil (1.14 g, crude).

### Preparation 29

### tert-butyl (3S,4R)-3-fluoro-4-(methoxy-d3)piperidine-1-carboxylate

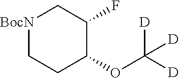 [SMILES: [2H]C([2H])([2H])O[C@@H]1CCN(C(=O)OC(C)(C)C)C[C@@H]1F]

The title compound was prepared from tert-butyl (3S,4R)-3-fluoro-4-hydroxypiperidine-1-carboxylate using an analogous method to that described for Preparation 28.

### Preparation 30

### 2-((3R,4S)-3-fluoro-4-(methoxy-d3)piperidin-1-yl)pyrimidin-4-amine

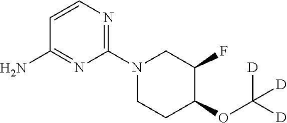 [SMILES: [2H]C([2H])([2H])O[C@H]1CCN(c2nccc(N)n2)C[C@H]1F]

TFA (2 mL) was added to tert-butyl (3R,4S)-3-fluoro-4-(methoxy-d₃)piperidine-1-carboxylate (Preparation 28, 1.14 g, 4.82 mmol) in DCM (6 mL) and the solution stirred for 2 h at rt. The mixture was evaporated to dryness in vacuo. The resulting residue was dissolved in IPA (20 mL), 2-chloropyrimidin-4-amine (496 mg, 3.83 mmol) and TEA (0.6 mL) added and the reaction mixture stirred overnight at 100° C. The mixture was evaporated to dryness and the residue purified by column chromatography (5% MeOH in EtOAc) to afford the title compound as a light-yellow solid (425 mg, 38.3%). LCMS m/z=230 [M+H]⁺.

### Preparation 31

### 2-((3S,4R)-3-fluoro-4-(methoxy-d3)piperidin-1-yl)pyrimidin-4-amine

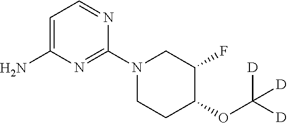 [SMILES: [2H]C([2H])([2H])O[C@@H]1CCN(c2nccc(N)n2)C[C@@H]1F]

The title compound was prepared from tert-butyl (3S,4R)-3-fluoro-4-(methoxy-d3)piperidine-1-carboxylate (Preparation 29) using an analogous method to that described for Preparation 30. LCMS m/z=230 [M+H]⁺.

### Preparation 32

### 2-((3R,4S)-3-fluoro-4-methoxypiperidin-1-yl)pyrimidin-4-amine

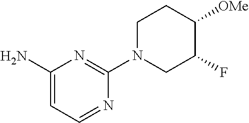 [SMILES: CO[C@H]1CCN(c2nccc(N)n2)C[C@H]1F]

Part 1: NaH (152 mg, 3.82 mmol) was added to a solution of tert-butyl (3R,4S)-3-fluoro-4-hydroxypiperidine-1-carboxylate (700 mg, 3.19 mmol) in THF (5 mL) at 0° C. Mel (497 mg, 3.5 mmol) was added and the mixture warmed to rt and stirred for 2 h. The reaction mixture was quenched with H₂O, extracted with EtOAc and washed with brine. The combined organics were dried (Na₂SO₄) and evaporated to dryness in vacuo to afford tert-butyl (3R,4S)-3-fluoro-4-methoxypiperidine-1-carboxylate as a yellow oil (750 mg, crude) which was used without further purification in Part 2.

Part 2: TFA (2 mL) was added to a solution of tert-butyl (3R,4S)-3-fluoro-4-methoxypiperidine-1-carboxylate (Part 1, 750 mg, 3.21 mmol) in DCM (10 mL) and the mixture stirred at rt for 3 h. The reaction mixture was evaporated to afford (3R,4S)-3-fluoro-4-methoxypiperidine trifluoroacetate as a brown oil (700 mg, crude).

Part 3: A mixture of (3R,4S)-3-fluoro-4-methoxypiperidine trifluoroacetate (Part 2, 700 mg, 5.25 mmol), 2-chloropyrimidin-4-amine (488 mg, 3.76 mmol) and DIPEA (1.44 g, 11.2 mmol) in DMSO (5 mL) was stirred at 100° C. for 2 h. The reaction mixture was diluted with water, extracted with EtOAc and washed with brine. The combined organics were dried (Na₂SO₄) and evaporated to dryness. The residue was purified by column chromatography (50% EtOAc in PE) to afford the title compound as a yellow solid (550 mg).

### Preparation 33

### 2-((3S,4R)-3-fluoro-4-methoxypiperidin-1-yl)pyrimidin-4-amine

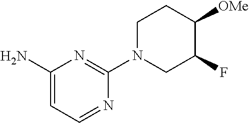 [SMILES: CO[C@@H]1CCN(c2nccc(N)n2)C[C@@H]1F]

The title compound was prepared from (3S,4R)-3-fluoropiperidin-4-ol using an analogous method to that described for Preparation 32. LCMS m/z=227 [M+H]⁺. ¹H NMR (400 MHz, DMSO-d₆) δ: 7.72 (d, 1H), 6.39 (s, 2H), 5.71 (d, 1H), 4.83 (d, 1H), 4.60-4.49 (m, 1H), 4.29 (d, 1H), 3.55-3.42 (m, 1H), 3.28 (d, 1H), 3.20-3.04 (m, 1H), 1.76-1.48 (m, 2H).

### Preparation 34

### tert-butyl 3-fluoro-4-hydroxy-3,4-dimethylpiperidine-1-carboxylate

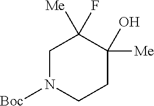 [SMILES: CC(C)(C)OC(=O)N1CCC(C)(O)C(C)(F)C1]

MeLi (27 mL, 43.2 mmol) was added to a mixture of tert-butyl 3-fluoro-3-methyl-4-oxopiperidine-1-carboxylate (Preparation 2, 5 g, 21.6 mmol) in THF at 0° C. and the reaction stirred at 0° C. for 1 h. The reaction was quenched with H₂O and extracted with EtOAc. The organic layer was evaporated under reduced pressure to afford a colorless oil, 6 g, crude, that was used in the next step directly.

### Preparation 35

### 3-fluoro-4-hydroxy-3,4-dimethylpiperidinol trifluoroacetate

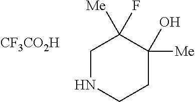

tert-Butyl 3-fluoro-4-hydroxy-3,4-dimethylpiperidine-1-carboxylate (Preparation 34, 6 g, crude) was placed in DCM/TFA (50 mL/15 mL) and the reaction stirred at rt for 1 h. The solvent was removed by evaporation to give the title compound, 6 g, crude, that was used without further purification.

### Preparation 36

### 1-(4-aminopyrimidin-2-yl)-3-fluoro-3,4-dimethylpiperidin-4-ol

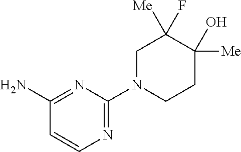 [SMILES: CC1(O)CCN(c2nccc(N)n2)CC1(C)F]

DIPEA (7.85 g, 60.9 mmol) was added to 3-fluoro-4-hydroxy-3,4-dimethylpiperidinol trifluoroacetate (Preparation 35, 3 g, crude) and 2-chloropyrimidin-4-amine (2.62 g, 20.3 mmol) in DMSO (20 mL) and the reaction stirred at 100° C. for 16 h. Water was added and the suspension was extracted with EtOAc. The organic phase was concentrated in vacuo and the residue was purified by silica gel chromatography (50% EtOAc in PE) to afford the title compound as a light-yellow solid (1.5 g).

### Preparation 37 and 38

### Rac-cis-1-(4-aminopyrimidin-2-yl)-3-fluoro-3,4-dimethylpiperidin-4-ol and rac-trans-1-(4-aminopyrimidin-2-yl)-3-fluoro-3,4-dimethylpiperidin-4-ol

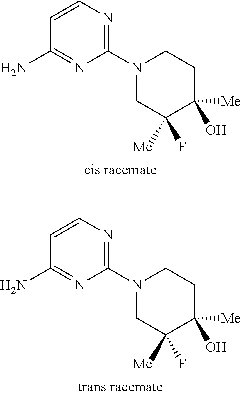 [SMILES: C[C@]1(F)CN(c2nccc(N)n2)CC[C@@]1(C)O ; C[C@@]1(O)CCN(c2nccc(N)n2)C[C@@]1(C)F]

1-(4-Aminopyrimidin-2-yl)-3-fluoro-3,4-dimethylpiperidin-4-ol (Preparation 36, 1.5 g, 6.24 mmol) was separated by SFC (CHIRALPAK IC-3, 3×100 mm 3 μm; MeOH (0.1% DEA) at 2 mL/min to afford

Peak 1, Preparation 37: rac-cis-1-(4-aminopyrimidin-2-yl)-3-fluoro-3,4-dimethylpiperidin-4-ol (identified as cis by 2D NMR, 400 mg) as a white solid and

Peak 2, Preparation 38: rac-trans-1-(4-aminopyrimidin-2-yl)-3-fluoro-3,4-dimethylpiperidin-4-ol (identified as trans by 2D NMR, 300 mg) as a white solid. LCMS m/z=241 [M+H]⁺.

### Preparation 39

### tert-butyl 3-fluoro-4-methoxy-3-methylpiperidine-1-carboxylate

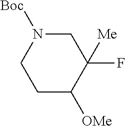 [SMILES: COC1CCN(C(=O)OC(C)(C)C)CC1(C)F]

The title compound was obtained as a colorless oil, 5.1 g, crude, from tert-butyl 3-fluoro-4-hydroxy-3-methylpiperidine-1-carboxylate (Preparation 3), following a similar procedure to that described in Preparation 32, Part 1.

### Preparations 40, 41, 42 and 43

### 2-((3S,4R)-3-fluoro-4-methoxy-3-methylpiperidin-1-yl)pyrimidin-4-amine, 2-((3R,4S)-3-fluoro-4-methoxy-3-methylpiperidin-1-yl)pyrimidin-4-amine, 2-((3R,4R)-3-fluoro-4-methoxy-3-methylpiperidin-1-yl)pyrimidin-4-amine and 2-((3S,4S)-3-fluoro-4-methoxy-3-methylpiperidin-1-yl)pyrimidin-4-amine

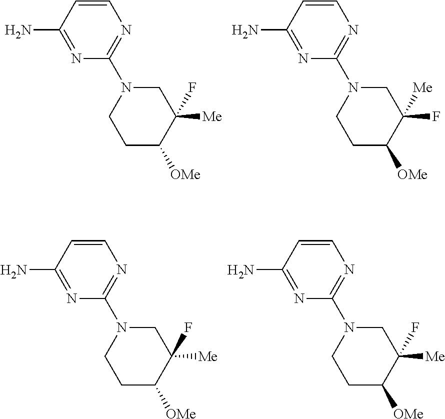 [SMILES: CO[C@@H]1CCN(c2nccc(N)n2)C[C@]1(C)F ; CO[C@H]1CCN(c2nccc(N)n2)C[C@@]1(C)F ; CO[C@@H]1CCN(c2nccc(N)n2)C[C@@]1(C)F ; CO[C@H]1CCN(c2nccc(N)n2)C[C@]1(C)F]

tert-Butyl 3-fluoro-4-methoxy-3-methylpiperidine-1-carboxylate (Preparation 39, 5.1 g) was dissolved in HCl/dioxane (4M, 50 mL) and the reaction stirred at rt for 2 h. The mixture was concentrated in vacuo and the residue was mixed with 2-chloropyrimidin-4-amine (4.21 g, 32.5 mmol) and TEA (5.47 g, 54.2 mmol) in IPA (30 mL). The mixture was stirred at 100° C. for 16 h and the cooled reaction was concentrated in vacuo. The residue was purified by column chromatography on silica gel (DCM:MeOH=10:1) to give 2-(3-fluoro-4-methoxy-3-methylpiperidin-1-yl)pyrimidin-4-amine, 1.6 g as a white solid. LCMS m/z=241 [M+H]⁺

This was further separated into four isomers using a CHIRAL ND(2) 4.6×100 mm, 3 μm column, eluting with MeOH (0.1% DEA) from 10% to 50% to afford:

Peak 1: 2-((3S,4R)-3-fluoro-4-methoxy-3-methylpiperidin-1-yl)pyrimidin-4-amine or 2-((3R,4S)-3-fluoro-4-methoxy-3-methylpiperidin-1-yl)pyrimidin-4-amine (490 mg)

Peak 2: 2-((3R,4S)-3-fluoro-4-methoxy-3-methylpiperidin-1-yl)pyrimidin-4-amine or 2-((3S,4R)-3-fluoro-4-methoxy-3-methylpiperidin-1-yl)pyrimidin-4-amine (440 mg) as pale-yellow solids.

Peak 3: 2-((3R,4R)-3-fluoro-4-methoxy-3-methylpiperidin-1-yl)pyrimidin-4-amine or 2-((3S,4S)-3-fluoro-4-methoxy-3-methylpiperidin-1-yl)pyrimidin-4-amine (81 mg) and

Peak 4: 2-((3S,4S)-3-fluoro-4-methoxy-3-methylpiperidin-1-yl)pyrimidin-4-amine or 2-((3R,4R)-3-fluoro-4-methoxy-3-methylpiperidin-1-yl)pyrimidin-4-amine (123 mg) as pale-yellow solids.

### Preparation 44

### tert-butyl 3-fluoro-3-methyl-4-((triethylsilyl)oxy)-3,6-dihydropyridine-1(2H)-carboxylate

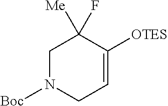

To a solution of tert-butyl 3-fluoro-3-methyl-4-oxopiperidine-1-carboxylate (4.7 g, 20.3 mmol) in THF (30 mL) was added LiHDMS (30.4 mL, 30.4 mmol) at −70° C. and the reaction allowed to warm to rt over 1 h. TESCl (6.11 g, 40.6 mmol) was added and the reaction stirred at rt for 2 h. The reaction was quenched with water and extracted with EtOAc. The organic layer was dried over Na₂SO₄, filtered and concentrated in vacuo. The residue was purified by silica gel column with (10% EtOAc:PE), to afford the title compound (6.2 g, 88%) as a colorless oil.

### Preparation 45

### tert-butyl 3,5-difluoro-3-methyl-4-oxopiperidine-1-carboxylate

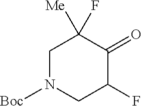 [SMILES: CC(C)(C)OC(=O)N1CC(F)C(=O)C(C)(F)C1]

To a solution of tert-butyl 3-fluoro-3-methyl-4-((triethylsilyl)oxy)-3,6-dihydropyridine-1(2H)-carboxylate (Preparation 44, 6.2 g, 17.9 mmol) in DMF (30 mL) at 10° C. was added SelectFluor (12.6 g, 35.8 mmol) and the reaction stirred at rt for 2 h. The reaction was quenched with water, extracted with EtOAc, the organic layer was dried over Na₂SO₄, filtered and concentrated in vacuo. The residue was purified by silica gel column with 30% EtOAc in PE to afford the title compound (3 g, 67%) as a light-yellow oil.

### Preparation 46

### tert-butyl 3,5-difluoro-4-hydroxy-3-methylpiperidine-1-carboxylate

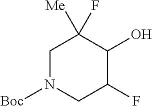 [SMILES: CC(C)(C)OC(=O)N1CC(F)C(O)C(C)(F)C1]

The title compound was obtained as a colorless oil, 2.9 g, 82.6% from tert-butyl 3,5-difluoro-3-methyl-4-oxopiperidine-1-carboxylate (Preparation 45), following the procedure described in Preparation 3. LCMS m/z=196 [M+H]⁺

### Preparation 47

### 3,5-difluoro-3-methylpiperidin-4-ol trifluoroacetate

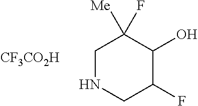

tert-Butyl 3,5-difluoro-4-hydroxy-3-methylpiperidine-1-carboxylate (Preparation 46, 1.6 g, 6.36 mmol) was added to a solution of DCM (20 mL) and TFA (5 mL) and the reaction stirred at rt for 2 h. The mixture was evaporated under reduced pressure to afford the title compound, 1.6 g.

### Preparations 48, 49, 50 and 51

### (3S,4R,5R)-1-(4-aminopyrimidin-2-yl)-3,5-difluoro-3-methylpiperidin-4-ol and (3R,4S,5S)-1-(4-aminopyrimidin-2-yl)-3,5-difluoro-3-methylpiperidin-4-ol and (3S,4S,5S)-1-(4-aminopyrimidin-2-yl)-3,5-difluoro-3-methylpiperidin-4-ol and (3R,4R,5R)-1-(4-aminopyrimidin-2-yl)-3,5-difluoro-3-methylpiperidin-4-ol

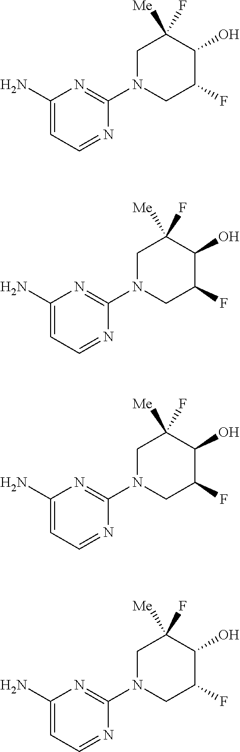 [SMILES: C[C@]1(F)CN(c2nccc(N)n2)C[C@@H](F)[C@H]1O ; C[C@@]1(F)CN(c2nccc(N)n2)C[C@H](F)[C@@H]1O ; C[C@]1(F)CN(c2nccc(N)n2)C[C@H](F)[C@@H]1O ; C[C@@]1(F)CN(c2nccc(N)n2)C[C@@H](F)[C@H]1O]

Into three 40-mL sealed tubes, was placed 2-chloropyrimidin-4-amine (900 mg, 5.95 mmol) in DMSO (10 mL), 3,5-difluoro-3-methylpiperidin-4-ol (Preparation 47, 1.6 g, crude) and DIPEA (3.07 g, 23.8 mmol) and the solution stirred for 24 h at 120° C. The reaction mixture was cooled to rt, diluted with water and extracted with EtOAc and the combined organic phases concentrated in vacuo. The residue was purified using HPLC-21. This resulted in 380 mg of (3S,4R,5R)-1-(4-aminopyrimidin-2-yl)-3,5-difluoro-3-methylpiperidin-4-ol and (3R,4S,5S)-1-(4-aminopyrimidin-2-yl)-3,5-difluoro-3-methylpiperidin-4-ol and 350 mg (3S,4S,5S)-1-(4-aminopyrimidin-2-yl)-3,5-difluoro-3-methylpiperidin-4-ol and (3R,4R,5R)-1-(4-aminopyrimidin-2-yl)-3,5-difluoro-3-methylpiperidin-4-ol as a white solid.

(3S,4R,5R)-1-(4-Aminopyrimidin-2-yl)-3,5-difluoro-3-methylpiperidin-4-ol and (3R,4S,5S)-1-(4-aminopyrimidin-2-yl)-3,5-difluoro-3-methylpiperidin-4-ol were further separated by SFC to give:

Peak 1: (3S,4R,5R)-1-(4-aminopyrimidin-2-yl)-3,5-difluoro-3-methylpiperidin-4-ol or (3R,4S,5S)-1-(4-aminopyrimidin-2-yl)-3,5-difluoro-3-methylpiperidin-4-ol, 150 mg

Peak 2: (3R,4S,5S)-1-(4-aminopyrimidin-2-yl)-3,5-difluoro-3-methylpiperidin-4-ol or (3S,4R,5R)-1-(4-aminopyrimidin-2-yl)-3,5-difluoro-3-methylpiperidin-4-ol, 150 mg.

(3S,4S,5S)-1-(4-Aminopyrimidin-2-yl)-3,5-difluoro-3-methylpiperidin-4-ol and (3R,4R,5R)-1-(4-aminopyrimidin-2-yl)-3,5-difluoro-3-methylpiperidin-4-ol was further separated by SFC to give:

Peak 3: (3S,4S,5S)-1-(4-aminopyrimidin-2-yl)-3,5-difluoro-3-methylpiperidin-4-ol or (3R,4R,5R)-1-(4-aminopyrimidin-2-yl)-3,5-difluoro-3-methylpiperidin-4-ol and

Peak 4: (3R,4R,5R)-1-(4-aminopyrimidin-2-yl)-3,5-difluoro-3-methylpiperidin-4-ol or (3S,4S,5S)-1-(4-aminopyrimidin-2-yl)-3,5-difluoro-3-methylpiperidin-4-ol, 140 mg. LCMS m/z=245 [M+H]⁺

### Preparation 52

### Rac-1-(4-aminopyrimidin-2-yl)-3,3-difluoro-4-methylpiperidin-4-ol

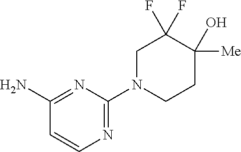 [SMILES: CC1(O)CCN(c2nccc(N)n2)CC1(F)F]

A mixture of 3,3-difluoro-4-methylpiperidin-4-ol (300 mg, 2.0 mmol), 2-chloropyrimidin-4-amine (260 mg, 2.0 mmol) and TEA (300 mg, 3.0 mmol) in DMSO (2 mL) was stirred overnight at 120° C. Water was added and the mixture extracted with EtOAc. The organic phase was washed with brine, dried and purified by column chromatography on silica gel (5% MeOH in DCM) to give 320 mg (65%) of the title compound as white solid. LCMS m/z=245 [M+H]⁺.

### Preparation 53

### Cis-rac-tert-butyl 3-hydroxy-4-methoxypiperidine-1-carboxylate

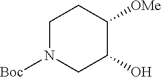 [SMILES: CO[C@H]1CCN(C(=O)OC(C)(C)C)C[C@H]1O]

Di-tert-butyl dicarbonate (1.66 g, 7.62 mmol) was added to cis-rac-4-methoxypiperidin-3-ol (500 mg, 3.81 mmol) and TEA (769 mg, 7.62 mmol) in DCM (20 mL) at 0° C. and the reaction stirred at rt for 16 h. The mixture was diluted with DCM (100 mL) and washed with brine (50 mL×2), the organic layer was dried over Na₂SO₄ and concentrated in vacuo. The residue was purified by silica gel column with PE:EtOAc=2:1 to give the title compound, 600 mg, as a colorless oil. LCMS m/z=176 [M−56]⁺

### Preparation 54

### Cis-rac-tert-butyl 3-(2-((tert-butyldimethylsilyl)oxy)ethoxy)-4-methoxypiperidine-1-carboxylate

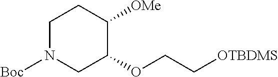 [SMILES: CO[C@H]1CCN(C(=O)OC(C)(C)C)C[C@H]1OCCO[Si](C)(C)C(C)(C)C]

The title compound was obtained as a colorless oil, 700 mg, 69.3 yield, from cis-rac-tert-butyl 3-hydroxy-4-methoxypiperidine-1-carboxylate (Preparation 53) following a similar procedure to that described in Preparation 24. LCMS m/z=390 [M+H]⁺

### Preparation 55

### Cis-rac-3-(2-((tert-butyldimethylsilyl)oxy)ethoxy)-4-methoxypiperidine trifluoroacetate

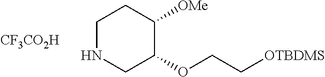 [SMILES: "CF3CO2H" ; CO[C@H]1CCNC[C@H]1OCCO[Si](C)(C)C(C)(C)C]

The title compound was obtained as a yellow oil, 300 mg, from cis-rac-tert-butyl 3-(2-((tert-butyldimethylsilyl)oxy)ethoxy)-4-methoxypiperidine-1-carboxylate (Preparation 54), following the procedure described in Preparation 35. LCMS m/z=176 [M+H]⁺

### Preparation 56

### Cis-rac-2-((1-(4-aminopyrimidin-2-yl)-4-methoxypiperidin-3-yl)oxy)ethan-1-ol

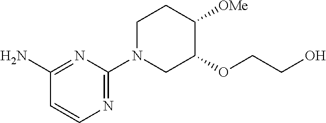 [SMILES: CO[C@H]1CCN(c2nccc(N)n2)C[C@H]1OCCO]

The title compound was obtained as a white solid, 300 mg, 65.2%, from 2-chloropyrimidin-4-amine and cis-rac-3-(2-((tert-butyldimethylsilyl)oxy)ethoxy)-4-methoxypiperidine trifluoroacetate (Preparation 55) following a similar procedure to that described in Preparation 24. LCMS m/z=269 [M+H]⁺

### Preparation 57

### 1-(4-aminopyrimidin-2-yl)-3,3-dimethylpiperidin-4-ol

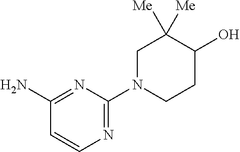 [SMILES: CC1(C)CN(c2nccc(N)n2)CCC1O]

The title compound was obtained, 600 mg, 35.3% yield, from 2-chloropyrimidin-4-amine and 3,3-dimethylpiperidin-4-ol, following a similar procedure to that described in Preparation 15. LCMS m/z=223 [M+H]⁺

### Preparation 58

### tert-butyl 8-hydroxy-5-azaspiro[2.5]octane-5-carboxylate

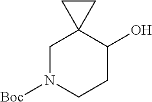 [SMILES: CC(C)(C)OC(=O)N1CCC(O)C2(CC2)C1]

To a solution of tert-butyl 8-oxo-5-azaspiro[2.5]octane-5-carboxylate (300 mg, 1.33 mmol) in EtOH (20 mL) was added NaBH₄ (150 mg, 3.99 mmol). The mixture was stirred at rt for 2 h, quenched with water and extracted with EtOAc. The organic layer was dried over Na₂SO₄, filtered and evaporated to dryness to afford the title compound, 300 mg as a colorless oil. LCMS m/z=227 [M+H]⁺

### Preparation 59

### 5-azaspiro[2.5]octan-8-ol trifluoroacetate

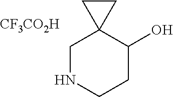

The title compound was obtained as a yellow oil, 350 mg, crude, from tert-butyl 8-hydroxy-5-azaspiro[2.5]octane-5-carboxylate (Preparation 58) following the procedure described in Preparation 35. LCMS m/z=127 [M+H]⁺

### Preparation 60

### 5-(4-aminopyrimidin-2-yl)-5-azaspiro[2.5]octan-8-ol

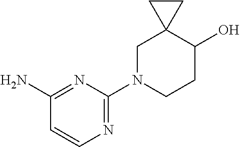 [SMILES: Nc1ccnc(N2CCC(O)C3(CC3)C2)n1]

A mixture of 2-chloropyrimidin-4-amine (165 mg, 1.28 mmol), 5-azaspiro[2.5]octan-8-ol trifluoroacetate (Preparation 59, 350 mg, 1.28 mmol) and DIPEA (496 mg, 3.84 mmol) in IPA (20 mL) was stirred at 100° C. for 2 h. The solution was concentrated in vacuo and the residue was purified by prep-TLC (DCM:MeOH=10:1) to afford the title compound, 120 mg, as a light-yellow oil. LCMS m/z=220 [M+H]⁺

### Preparation 61

### 1-(4-aminopyrimidin-2-yl)-4-(hydroxymethyl)piperidin-4-ol

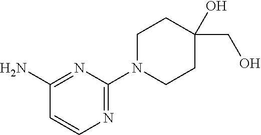 [SMILES: Nc1ccnc(N2CCC(O)(CO)CC2)n1]

A mixture of 2-chloropyrimidin-4-amine (0.1 g, 0.772 mmol), 4-(hydroxymethyl)piperidin-4-ol hydrochloride (155 mg, 0.926 mmol) and DIPEA (249 mg, 1.93 mmol) in IPA (4 mL) was heated to 100° C. overnight. The cooled mixture was concentrated in vacuo and the residue partitioned between 5% MeOH/DCM and water and the layers separated. The organic phase was washed with water, dried over Na₂SO₄, filtered and evaporated to give the title compound, 55 mg as yellow semi-solid. LCMS m/z=225 [M+H]⁺

### Preparation 62

### Rac-2-(1-oxa-7-azaspiro[3.5]nonan-7-yl)pyrimidin-4-amine

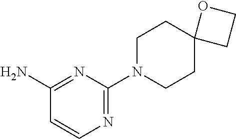 [SMILES: Nc1ccnc(N2CCC3(CCO3)CC2)n1]

A mixture of 1-oxa-7-azaspiro[3.5]nonane (1.27 g, 10 mmol), DIPEA (2.6 g, 20 mmol) and 2-chloropyrimidin-4-amine (1.29 g, 10 mmol) in DMSO (12 mL) was stirred overnight at 120° C. The mixture was cooled to rt and diluted with water. The resulting suspension was extracted with EtOAc, the organic layers were washed with brine, dried and concentrated in vacuo. The residue was purified by prep-TLC to afford the title compound, 1.3 g, 59% as pale-yellow solid. LCMS m/z=221 [M+H]⁺.

### Preparation 63

### tert-butyl 5,5-difluoro-1-oxa-7-azaspiro[3.5]nonane-7-carboxylate

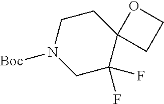 [SMILES: CC(C)(C)OC(=O)N1CCC2(CCO2)C(F)(F)C1]

Tert-butyl 3,3-difluoro-4-oxopiperidine-1-carboxylate (2 g, 8.50 mmol), trimethylsulfoxonium iodide (5.61 g, 25.5 mmol) and t-BuOK (2.85 g, 25.5 mmol) was dissolved in t-BuOH and the reaction stirred at 50° C. for 2 days. Water was added, the reaction mixture extracted with EtOAc and the combined organic layers were evaporated under reduced pressure to afford the title compound as yellow solid.

### Preparation 64

### 5,5-difluoro-1-oxa-7-azaspiro[3.5]nonane trifluoroacetate

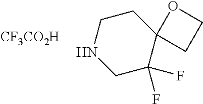 [SMILES: "CF3CO2H" ; FC1(F)CNCCC12CCO2]

The title compound was obtained from tert-butyl 5,5-difluoro-1-oxa-7-azaspiro[3.5]nonane-7-carboxylate (Preparation 63) following the procedure described in Preparation 35. LCMS m/z=164 [M+H]⁺

### Preparation 65

### 2-(5,5-difluoro-1-oxa-7-azaspiro[3.5]nonan-7-yl)pyrimidin-4-amine

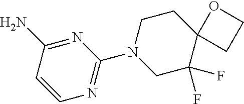 [SMILES: Nc1ccnc(N2CCC3(CCO3)C(F)(F)C2)n1]

The title compound was obtained as a yellow solid, 9.5 g, 60.4% yield, from 5,5-difluoro-1-oxa-7-azaspiro[3.5]nonane trifluoroacetate (Preparation 64) and 2-chloropyrimidin-4-amine, following a similar procedure to that described in Preparation 52. LCMS m/z=257 [M+H]⁺

### Preparations 66 and 67

### (R)-2-(5,5-difluoro-1-oxa-7-azaspiro[3.5]nonan-7-yl)pyrimidin-4-amine and (S)-2-(5,5-difluoro-1-oxa-7-azaspiro[3.5]nonan-7-yl)pyrimidin-4-amine

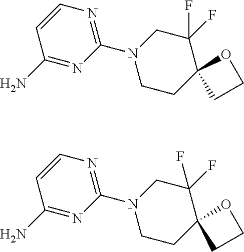 [SMILES: Nc1ccnc(N2CC[C@@]3(CCO3)C(F)(F)C2)n1 ; Nc1ccnc(N2CC[C@]3(CCO3)C(F)(F)C2)n1]

2-(5,5-Difluoro-1-oxa-7-azaspiro[3.5]nonan-7-yl)pyrimidin-4-amine (Preparation 65, 1.0 g) was purified by Prep-SFC using a CHIRALART Amylose-SA, 2×25 cm, 5 μm column, mobile phase: 35% EtOH; to afford the title enantiomers.

Peak 1: (R)-2-(5,5-difluoro-1-oxa-7-azaspiro[3.5]nonan-7-yl)pyrimidin-4-amine or (S)-2-(5,5-difluoro-1-oxa-7-azaspiro[3.5]nonan-7-yl)pyrimidin-4-amine (450 mg)

1H NMR (400 MHz, DMSO-d₆) δ: 7.74 (d, 1H), 6.52 (s, 2H), 5.77 (d, 1H), 4.46 (t, 2H), 4.23 (td, 1H), 3.93-3.74 (m, 2H), 3.53 (ddd, 1H), 2.74 (dt, 1H), 2.50-2.39 (m, 1H), 2.10-1.99 (m, 1H), 1.90 (ddt, 1H).

Peak 2: (S)-2-(5,5-difluoro-1-oxa-7-azaspiro[3.5]nonan-7-yl)pyrimidin-4-amine or (R)-2-(5,5-difluoro-1-oxa-7-azaspiro[3.5]nonan-7-yl)pyrimidin-4-amine (430 mg).

1H NMR (400 MHz, DMSO-d₆) δ 7.75 (d, 1H), 6.52 (s, 2H), 5.77 (d, 1H), 4.46 (t, 2H), 4.23 (td, 1H), 3.94-3.74 (m, 2H), 3.53 (ddd, 1H), 2.74 (dt, 1H), 2.50-2.41 (m, 1H), 2.10-1.97 (m, 1H), 1.91 (ddt, 1H).

### Preparation 68

### 1-(5-amino-1,2,4-triazin-3-yl)-3-fluoro-3-methylpiperidin-4-ol

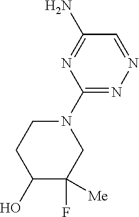 [SMILES: CC1(F)CN(c2nncc(N)n2)CCC1O]

To a solution of 3-chloro-1,2,4-triazin-5-amine (120 mg, 0.42 mmol) and TEA (126 mg, 1.25 mmol) in IPA was added 3-fluoro-3-methylpiperidin-4-ol (55.9 mg, 0.42 mmol) and the reaction stirred for 2 h at 100° C. The mixture was washed with water, extracted with EtOAc, the combined organic layer dried over Na₂SO₄, and concentrated in vacuo. The residue was purified by prep-TLC (PE/EtOAc (5:1) to give the title compound, 50 mg (52%) as a light yellow solid. LCMS m/z=228 [M+H]⁺

### Preparation 69

### tert-butyl 3-(((methylsulfonyl)oxy)methyl)azetidine-1-carboxylate

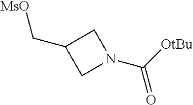 [SMILES: CC(C)(C)OC(=O)N1CC(COS(C)(=O)=O)C1]

Tert-butyl 3-(hydroxymethyl)azetidine-1-carboxylate (1.06 g, 5.66 mmol) in DCM (20 mL) was cooled to 0° C., TEA (1.184 mL, 8.49 mmol) added, followed by mesyl chloride (0.485 mL, 6.23 mmol) dropwise, and the reaction allowed to warm slowly to rt. The reaction was diluted with DCM, washed with water (×2) and dried over Na₂SO₄. The mixture was filtered, and evaporated under reduced pressure to give the title compound, 1.21 g as a pale yellow oil.

### Preparation 70

### tert-butyl 3-((1H-1,2,3-triazol-1-yl)methyl)azetidine-1-carboxylate

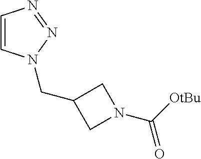 [SMILES: CC(C)(C)OC(=O)N1CC(Cn2ccnn2)C1]

1H-1,2,3-Triazole (0.2 mL, 3.39 mmol) was added to a solution of tert-butyl 3-(((methylsulfonyl)oxy)methyl)azetidine-1-carboxylate (Preparation 69, 750 mg, 2.83 mmol) and K₂CO₃ (391 mg, 2.83 mmol) in DMF (10.47 mL) under N₂ and the reaction stirred to 85° C. for 5 h. The reaction was poured into ice water, EtOAc added, the layers separated and the organic layer washed with water (3×) and brine. The organic layer was dried over anhydrous Na₂SO₄, filtered and concentrated in vacuo. The crude product was purified by silica gel chromatography (Combiflash®) eluting with 0-50% EtOAc in hexanes to give tert-butyl 3-((2H-1,2,3-triazol-2-yl)methyl)azetidine-1-carboxylate. Further elution provided the title compound as a colorless oil, 163 mg, 24.2% yield.

¹H NMR (400 MHz, DMSO-d₆) δ: 8.19 (d, 1H), 7.75 (d, 1H), 4.64 (d, 2H), 3.92 (t, 2H), 3.69 (t, 2H), 3.16-2.95 (m, 1H), 1.40 (d, 9H).

### Preparation 71

### (2R,3S)-1-benzhydryl-2-methylazetidin-3-ol ((1S)-7,7-dimethyl-2-oxobicyclo[2.2.1]heptan-1-yl)methanesulfonate

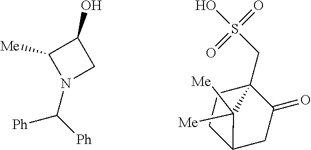 [SMILES: C[C@@H]1[C@@H](O)CN1C(c1ccccc1)c1ccccc1 ; CC1(C)C2CC[C@@]1(CS(=O)(=O)O)C(=O)C2]

A solution of trans-rac-1-benzhydryl-2-methylazetidin-3-ol (240 g, 947 mmol) in EtOH (750 mL) was added to a solution of ((1S)-7,7-dimethyl-2-oxo-norbornan-1-yl)methanesulfonic acid (231 g, 995 mmol) in DCM (500 mL) and the reaction was stirred at 15° C. for 1 h. The reaction was concentrated in vacuo, and the residue azeotroped with DCM. The residue was suspended in DCM (800 mL) and the mixture stirred at 15° C. for 12 h. The suspension was filtered and the filter cake rinsed with DCM (100 mL). The solid was collected and dried under vacuum to give the title compound, 150 g, 32.6% yield as white solid.

### Preparation 72

### (2R,3S)-2-methylazetidin-3-ol ((1S)-7,7-dimethyl-2-oxobicyclo[2.2.1]heptan-1-yl)methanesulfonate

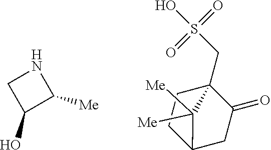 [SMILES: C[C@H]1NC[C@@H]1O ; CC1(C)C2CC[C@@]1(CS(=O)(=O)O)C(=O)C2]

A suspension of (2R,3S)-1-benzhydryl-2-methylazetidin-3-ol ((1S)-7,7-dimethyl-2-oxobicyclo[2.2.1]heptan-1-yl)methanesulfonate (Preparation 71, 150 g, 309 mmol) and Pd(OH)₂/C (30.0 g, 10% purity) in MeOH (800 mL) and DCM (100 mL) was stirred at 34° C. under 50 psi of H₂ for 6 h. The reaction suspension was filtered and the filter cake was rinsed with DCM/MeOH (1/1, 1 L). The combined filtrate was concentrated in vacuo and the crude was triturated with DCM/MTBE (1/2, 300 mL) and filtered. The filter cake was collected and dried in vacuo to give the title compound (75.0 g, 76% yield) as white solid. ¹H NMR (400 MHz, MeOD-d₄) δ: 4.34-4.27 (m, 2H), 4.09-4.04 (m, 1H), 3.80-3.76 (m, 1H), 3.31 (d, 1H), 2.80 (d, 1H), 2.69-2.62 (m, 1H), 2.38-2.34 (m, 1H), 2.09-2.04 (m, 2H), 1.91 (d, 1H), 1.68-1.63 (m, 1H), 1.54 (d, 3H), 1.47-1.41 (m, 1H), 1.13 (s, 3H), 0.88 (s, 3H)

### Preparation 73

### (2R,3S)-2-methylazetidin-3-ol trifluoroacetate

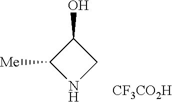

A mixture of (2R,3S)-1-(diphenylmethyl)-2-methylazetidin-3-ol (1 g, 3.94 mmol) and Pd(OH)₂ (30% w, 300 mg) in MeOH:TFA=30:1, (10 mL) was stirred under an atmosphere of H₂ at rt for 2 h. The mixture was filtered and the filtrate evaporated under reduced pressure to give the title compound, 300 mg, as a white solid.

### Preparation 74

### (2R,3S)-1-benzhydryl-2-methylazetidin-3-yl methanesulfonate

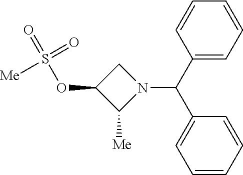 [SMILES: C[C@@H]1[C@@H](OS(C)(=O)=O)CN1C(c1ccccc1)c1ccccc1]

TEA (1.719 mL, 12.34 mmol) and methanesulfonyl chloride (0.802 mL, 10.36 mmol) were added slowly to a solution of (2R,3S)-1-benzhydryl-2-methylazetidin-3-ol (2.5 g, 9.87 mmol) in DCM (39.5 mL) at 0° C. and the reaction was allowed to warm to rt and stirred overnight. The reaction mixture was diluted with DCM and washed with water (2×10 mL). The organic layer was dried over Na₂SO₄, filtered and the solvent removed under reduced pressure to afford the title compound, as an orange oil, 3.23 g, 99% yield.

¹H NMR (400 MHz, DMSO-d₆) δ: 7.42 (dt, 4H), 7.34-7.24 (m, 4H), 7.24-7.15 (m, 2H), 4.63 (q, 1H), 4.53 (s, 1H), 3.60 (dd, 1H), 3.33 (d, 1H), 3.17 (s, 3H), 2.78 (dd, 1H), 0.71 (d, 3H)

### Preparation 75

### 1-((2R,3S)-1-benzhydryl-2-methylazetidin-3-yl)-1H-1,2,3-triazole

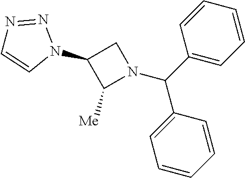 [SMILES: C[C@@H]1[C@@H](n2ccnn2)CN1C(c1ccccc1)c1ccccc1]

1H-1,2,3-Triazole (0.105 mL, 1.81 mmol) was added to a solution of (2R,3S)-1-benzhydryl-2-methylazetidin-3-yl methanesulfonate (Preparation 74, 500 mg, 1.509 mmol) and K₂CO₃ (208 mg, 1.509 mmol) in DMF (5.59 mL) and the reaction stirred at 85° C. under N₂ for 5 h. The reaction was poured into ice water, extracted with EtOAc, the combined organic extracts washed with water and then brine. The organic solution was dried over Na₂SO₄, filtered and concentrated in vacuo. The residue was purified by Isco Combiflash® (0-50% EtOAc in hexanes) to give the title compound, 208.5 mg, 45.4% yield, as a white solid.

¹H NMR (400 MHz, DMSO-d₆) δ: 7.83 (s, 2H), 7.51-7.44 (m, 4H), 7.34-7.28 (m, 4H), 7.23-7.17 (m, 2H), 4.85 (q, 1H), 4.59 (s, 1H), 3.74 (t, 1H), 3.66-3.57 (m, 1H), 3.24 (t, 1H), 0.77 (d, 3H).

### Preparation 76

### 1-((2R,3S)-2-methylazetidin-3-yl)-1H-1,2,3-triazole

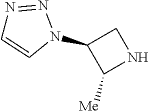

TFA (0.063 mL, 0.82 mmol) was added to a solution of 1-((2R,3S)-1-benzhydryl-2-methylazetidin-3-yl)-1H-1,2,3-triazole (Preparation 75, 208 mg, 0.683 mmol) and 20% Pd(OH)₂ (48.0 mg, 0.068 mmol) in MeOH (4.56 mL) and the reaction stirred overnight under an atmosphere of H₂. The reaction was filtered through Celite®, rinsed with Et₂O and evaporated under reduced pressure. The residue was washed with Et₂O and the solvent decanted off. The residue was dissolved in MeOH (10 mL), cooled to 0° C. and MP carbonate resin was added and the mixture stirred until pH˜9. The mixture was filtered and solvent removed under reduced pressure, to give the title compound, 31.9 mg, 33.8% as a colorless oil. LCMS m/z=139 [M+H]⁺

### Preparation 77

### Trans-rac-1-(tert-butoxycarbonyl)-2-methylazetidine-3-carboxylic acid

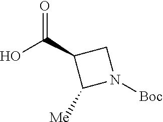 [SMILES: C[C@@H]1[C@@H](C(=O)O)CN1C(=O)OC(C)(C)C]

To a solution of trans-rac-2-methylazetidine-3-carboxylic acid (1 g, 8.68 mmol) and Na₂CO₃ (1.86 g, 17.3 mmol) in dioxane and water was added Boc₂O (3.77 g, 17.3 mmol) and the reaction stirred at rt for 2 h. The pH of the mixture was adjusted to pH<7 using 1M HCl and extracted with EtOAc. The combined organic layers were dried over Na₂SO₄, filtered and evaporated under reduced pressure to give the title compound. LCMS m/z=160 [M+H]⁺

### Preparation 78

### Trans-rac-tert-butyl 2-methyl-3-(2-(methylcarbamothioyl)hydrazine-1-carbonyl)azetidine-1-carboxylate

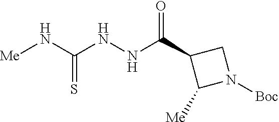 [SMILES: CNC(=S)NNC(=O)[C@H]1CN(C(=O)OC(C)(C)C)[C@@H]1C]

A mixture of trans-rac-1-(tert-butoxycarbonyl)-2-methylazetidine-3-carboxylic acid (Preparation 77, 800 mg, 3.71 mmol), 1-amino-3-methylthiourea (584 mg, 5.56 mmol), EDC (1.06 g, 5.56 mmol) and HOBt (750 mg, 5.56 mmol) in DMF was stirred at rt for 3 h. The mixture was partitioned between EtOAc and water and the layers separated. The organic layer was dried over Na₂SO₄ and concentrated in vacuo. The residue was purified by prep-TLC with PE/EtOAc (5:1) to give the title compound, 900 mg, 83% as a yellow oil. LCMS m/z=303 [M+H]⁺

### Preparation 79

### Trans-rac-tert-butyl 2-methyl-3-(4-methyl-5-thioxo-4,5-dihydro-1H-1,2,4-triazol-3-yl)azetidine-1-carboxylate

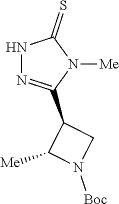 [SMILES: C[C@@H]1[C@@H](c2n[nH]c(=S)n2C)CN1C(=O)OC(C)(C)C]

A mixture of trans-rac-tert-butyl 2-methyl-3-(2-(methylcarbamothioyl)hydrazine-1-carbonyl)azetidine-1-carboxylate (Preparation 78, 800 mg, 2.64 mmol) in 1 M aq. NaOH was stirred at 60° C. overnight. The mixture was cooled to 0° C., and the mixture acidified to pH 6 using HCl. The resulting solid was filtered off and dried to give the title compound, as a white solid, 600 mg, 80%. LCMS m/z=285 [M+H]⁺

### Preparation 80

### Trans-rac-tert-butyl 2-methyl-3-(4-methyl-4H-1,2,4-triazol-3-yl)azetidine-1-carboxylate

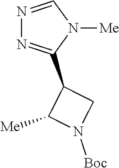 [SMILES: C[C@@H]1[C@@H](c2nncn2C)CN1C(=O)OC(C)(C)C]

A solution of trans-rac-tert-butyl 2-methyl-3-(4-methyl-5-thioxo-4,5-dihydro-1H-1,2,4-triazol-3-yl)azetidine-1-carboxylate (Preparation 79, 800 mg, 2.81 mmol) in DCM was cooled to 0° C., and a solution of H₂O₂ in AcOH was added in portions under cooling and stirring. The ice bath was removed, and the reaction stirred for a further 3 h at rt. The pH of the reaction mixture was adjusted to 10 with NaOH, the organic layer was separated and the aqueous was washed with DCM. The combined organic extracts were dried over Na₂SO₄ and evaporated under reduced pressure. The residue was purified by prep-TLC with DCM/MeOH (20:1) to give the title compound as a white solid, 400 mg, 56.3%. LCMS m/z=253 [M+H]⁺

### Preparation 81

### Trans-rac-4-methyl-3-(2-methylazetidin-3-yl)-4H-1,2,4-triazole trifluoroacetate

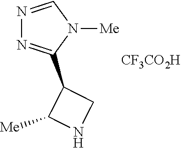 [SMILES: C[C@H]1NC[C@@H]1c1nncn1C ; "CF3CO2H"]

The title compound was obtained as a yellow oil, from trans-rac-tert-butyl 2-methyl-3-(4-methyl-4H-1,2,4-triazol-3-yl)azetidine-1-carboxylate (Preparation 80) following the procedure described in Preparation 64. LCMS m/z=153 [M+H]⁺

### Preparation 82

### tert-butyl (R)-(4-chloro-3-oxobutan-2-yl)carbamate

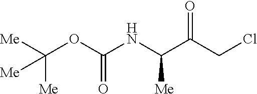 [SMILES: C[C@@H](NC(=O)OC(C)(C)C)C(=O)CCl]

A solution of tert-butyl chloride (0.56 g, 6.12 mmol) in THF (5 mL), followed by ethyl bromide (1.3 mL) were added to a flask containing magnesium (3 g, 0.12 mmol) and iodine (10 mg) and the reaction heated to 60° C. Additional tert-butyl chloride (10.7 g, 0.11 mol) in THF (60 mL) was added dropwise and after addition, the solution was stirred for 30 min at 60° C. The solution was cooled to 0° C., TEA (3 g, 30 mmol) and sodium chloroacetate (3.47 g, 30 mmol) were added. A solution of Boc-D-alanine methyl ester (3.9 g, 19 mmol) in toluene (30 mL) was then added dropwise and the reaction was warmed to rt and stirred overnight. The mixture was cooled to 0° C., and acetic acid (8 g, 0.13 mol) in water (16 mL) was added dropwise. Aqueous 2M HCl was added to adjust the aqueous phase to pH=4 and the mixture stirred at rt for 30 min. The mixture was extracted with EtOAc, washed with aqueous NaHCO₃ and brine, dried over Na₂SO₄ and evaporated under reduced pressure to give the title compound, 4.2 g as yellow oil.

### Preparation 83

### tert-butyl ((2R,3R)-4-chloro-3-hydroxy-3-methylbutan-2-yl)carbamate

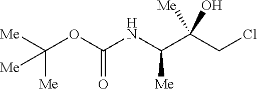 [SMILES: C[C@@H](NC(=O)OC(C)(C)C)[C@@](C)(O)CCl]

MeMgBr (18 mL, 3M in ether) was added dropwise to a solution of tert-butyl (R)-(4-chloro-3-oxobutan-2-yl)carbamate (Preparation 82, 3.6 g, 56 mmol) in DCM (80 mL) at −75° C. and the reaction stirred at −75° C. for 35 min and then allowed to warm to −5° C. over 3 h. The mixture was quenched with aq. NH₄Cl and the pH adjusted to 2 using 2N aq HCl. The mixture was extracted with DCM, dried over Na₂SO₄ and concentrated in vacuo to afford the title compound, 3.5 g as yellow solid.

### Preparation 84

### (2R,3S)-2,3-dimethylazetidin-3-ol

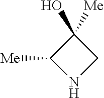

tert-Butyl ((2R,3R)-4-chloro-3-hydroxy-3-methylbutan-2-yl)carbamate (Preparation 83, 1 g, 4 mmol) was added to a solution of TFA (3 mL) in DCM (8 mL) and the reaction was stirred for 2 h at rt. The mixture was concentrated in vacuo, the residue was dissolved in MeCN (10 mL) and treated with solid NaHCO₃. The suspension was stirred for 2 h at 70° C., cooled to rt, the solid was filtered off and the filtrate was evaporated under reduced pressure to give the title compound, 600 mg, which was used directly for the next step.

### Preparation 85

### tert-butyl 3-(2-formylhydrazine-1-carbonyl)-3-methylazetidine-1-carboxylate

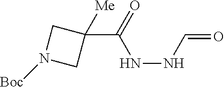 [SMILES: CC(C)(C)OC(=O)N1CC(C)(C(=O)NNC=O)C1]

A mixture of 1-(tert-butoxycarbonyl)-3-methylazetidine-3-carboxylic acid (1 g, 4.64 mmol), formic hydrazide (417 mg, 6.96 mmol), EDC (1.33 g, 6.96 mmol) and HOBt (939 mg, 6.96 mmol) in DMF was stirred at rt for 3 h. Water was added, the mixture extracted with DCM, the combined organic extracts were dried over Na₂SO₄ and concentrated in vacuo. The residue was purified by prep-TLC with PE/EtOAc (5:1) to give the title compound, 1 g, 84% as a yellow oil. LCMS m/z=258 [M+H]⁺

### Preparation 86

### tert-butyl 3-(2-formylhydrazine-1-carbonyl)azetidine-1-carboxylate

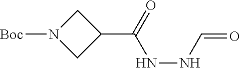 [SMILES: CC(C)(C)OC(=O)N1CC(C(=O)NNC=O)C1]

The title compound was obtained as a yellow oil, 1 g, 83.3% yield, from 1-(tert-butoxycarbonyl)azetidine-3-carboxylic acid, following a similar procedure to that described in Preparation 85. LCMS m/z=244 [M+H]⁺

### Preparation 87

### tert-butyl 3-(2-acetylhydrazine-1-carbonyl)azetidine-1-carboxylate

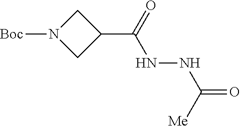 [SMILES: CC(=O)NNC(=O)C1CN(C(=O)OC(C)(C)C)C1]

The title compound was obtained as a yellow oil, 1 g, 78.7% yield, from 1-(tert-butoxycarbonyl)azetidine-3-carboxylic acid and acetic hydrazide, following a similar procedure to that described in Preparation 85. LCMS m/z=258 [M+H]⁺

### Preparation 88

### Trans-rac-tert-butyl 3-(2-formylhydrazine-1-carbonyl)-2-methylazetidine-1-carboxylate

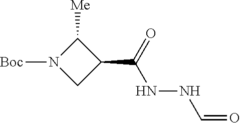 [SMILES: C[C@@H]1[C@@H](C(=O)NNC=O)CN1C(=O)OC(C)(C)C]

The title compound was obtained as a yellow oil, 820 mg, 86% yield, from trans-rac-1-(tert-butoxycarbonyl)-2-methylazetidine-3-carboxylic acid (Preparation 77) and formic hydrazide, following a similar procedure to that described in Preparation 85.

### Preparation 89

### tert-butyl 3-methyl-3-(1,3,4-oxadiazol-2-yl)azetidine-1-carboxylate

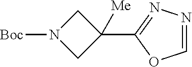 [SMILES: CC(C)(C)OC(=O)N1CC(C)(c2nnco2)C1]

To a solution of tert-butyl 3-(2-formylhydrazine-1-carbonyl)-3-methylazetidine-1-carboxylate (Preparation 85, 1.4 g, 5.44 mmol) and TEA (1.64 g, 16.3 mmol) in MeCN at 0° C. was added TsCl (1.55 g, 8.16 mmol) slowly under N₂, the reaction allowed to warm to rt and stirred for 2 h. Water was added and the mixture was extracted with EtOAc. The organic layers were combined, dried over Na₂SO₄ and concentrated in vacuo. The residue was purified by prep-TLC with PE/EtOAc (5:1) to give the title compound, 1 g, 77%, as a light-yellow liquid. LCMS m/z=240 [M+H]⁺

### Preparation 90

### tert-butyl 3-(1,3,4-oxadiazol-2-yl)azetidine-1-carboxylate

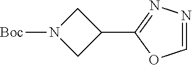 [SMILES: CC(C)(C)OC(=O)N1CC(c2nnco2)C1]

The title compound was obtained as a yellow oil, 500 mg, 90.2% yield, from tert-butyl 3-(2-formylhydrazine-1-carbonyl)azetidine-1-carboxylate (Preparation 86) following a similar procedure to that described in Preparation 89. LCMS m/z=226 [M+H]⁺

### Preparation 91

### tert-butyl 3-(5-methyl-1,3,4-oxadiazol-2-yl)azetidine-1-carboxylate

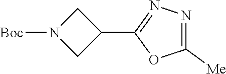 [SMILES: Cc1nnc(C2CN(C(=O)OC(C)(C)C)C2)o1]

The title compound was obtained as a light yellow solid, 400 mg, 86.2% yield, from tert-butyl 3-(2-acetylhydrazine-1-carbonyl)azetidine-1-carboxylate (Preparation 87) following a similar procedure to that described in Preparation 89. LCMS m/z=240 [M+H]⁺

### Preparation 92

### trans-rac-tert-butyl 2-methyl-3-(1,3,4-oxadiazol-2-yl)azetidine-1-carboxylate

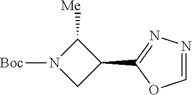 [SMILES: C[C@@H]1[C@@H](c2nnco2)CN1C(=O)OC(C)(C)C]

The title compound was obtained as a yellow oil, 600 mg, 81% yield, from trans-rac-tert-butyl 3-(2-formylhydrazine-1-carbonyl)-2-methylazetidine-1-carboxylate (Preparation 88) following a similar procedure to that described in Preparation 89.

### Preparation 93

### 2-(3-methylazetidin-3-yl)-1,3,4-oxadiazole trifluorocetate

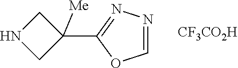

The title compound was obtained as a brown solid, 100 mg, 86% yield from tert-butyl 3-methyl-3-(1,3,4-oxadiazol-2-yl)azetidine-1-carboxylate (Preparation 89), following the procedure described in Preparation 64. LCMS m/z=140 [M+H]⁺

### Preparation 94

### 2-(azetidin-3-yl)-1,3,4-oxadiazole trifluoroacetate

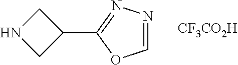

The title compound was obtained as a yellow oil, 100 mg, 91% yield from tert-butyl 3-(1,3,4-oxadiazol-2-yl)azetidine-1-carboxylate (Preparation 90), following the procedure described in Preparation 64. LCMS m/z=140 [M+H]⁺

### Preparation 95

### 2-(azetidin-3-yl)-5-methyl-1,3,4-oxadiazole trifluoroacetate

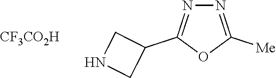

The title compound was obtained from tert-butyl 3-(5-methyl-1,3,4-oxadiazol-2-yl)azetidine-1-carboxylate (Preparation 91), following the procedure described in Preparation 64. LCMS m/z=140 [M+H]⁺

### Preparation 96

### trans-rac-2-(2-methylazetidin-3-yl)-1,3,4-oxadiazole trifluoroacetate

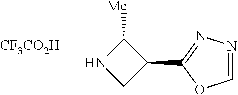

The title compound was obtained, 150 mg, 87% from trans-rac-tert-butyl 2-methyl-3-(1,3,4-oxadiazol-2-yl)azetidine-1-carboxylate (Preparation 92), following the procedure described in Preparation 64. LCMS m/z=140 [M+H]⁺

### Preparation 97

### benzyl 3-(dimethylcarbamoyl)-3-methylazetidine-1-carboxylate

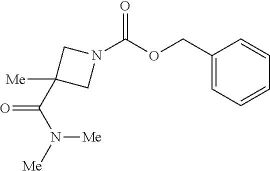 [SMILES: CN(C)C(=O)C1(C)CN(C(=O)OCc2ccccc2)C1]

A mixture of 1-((benzyloxy)carbonyl)-3-methylazetidine-3-carboxylic acid (250 mg, 1.003 mmol), dimethylamine (0.752 mL, 1.504 mmol), HATU (496 mg, 1.304 mmol) and DIPEA (324 mg, 2.507 mmol) in THF (4 mL) was stirred at rt for 72 h. The mixture was diluted with EtOAc, washed with dilute HCl, aq. NaHCO₃, brine and dried over Na₂SO₄. The mixture was filtered and concentrated in vacuo. The residue was purified by Isco chromatography (0-100% EtOAc/hexanes) to afford the title compound, 267 mg, 96.1% as a colorless oil. LCMS m/z=277 [M+H]⁺

### Preparation 98

### N,N,3-trimethylazetidine-3-carboxamide

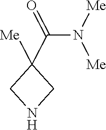

A mixture of benzyl 3-(dimethylcarbamoyl)-3-methylazetidine-1-carboxylate (Preparation 97) and 10% Pd/C (27 mg) in MeOH (6 mL) was stirred at rt under an atmosphere of H₂ for 90 min. The mixture was filtered through Celite® and the filtrate evaporated under reduced pressure to afford the title compound, as a colorless oil, 139 mg. LCMS/z=143 [M+H]⁺

### Preparation 99

### tert-butyl 3-(dimethylcarbamoyl)-3-fluoroazetidine-1-carboxylate

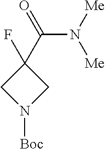 [SMILES: CN(C)C(=O)C1(F)CN(C(=O)OC(C)(C)C)C1]

The title compound was obtained as a colorless oil, 265 mg, 92%, from 1-(tert-butoxycarbonyl)-3-fluoroazetidine-3-carboxylic acid and dimethylamine, following the procedure described in Preparation 97.

### Preparation 100

### 3-fluoro-N,N-dimethylazetidine-3-carboxamide

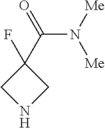

A solution of tert-butyl 3-(dimethylcarbamoyl)-3-fluoroazetidine-1-carboxylate (Preparation 99, 265 mg, 1.076 mmol) in TFA (0.829 mL) and DCM (4 mL) was stirred at rt for 3 h. The mixture was concentrated in vacuo and azeotroped twice with DCM. The residue was re-dissolved in DCM, 1.5 g of carbonate resin (˜3.5 mmol/g) was added and the mixture stirred for 15 min. The mixture was filtered and evaporated under reduced pressure to give the title compound, as a viscous yellow oil, 122 mg, 78%. LCMS m/z=147 [M+H]⁺

### Preparation 101

### benzyl 3-(((trifluoromethyl)sulfonyl)oxy)azetidine-1-carboxylate

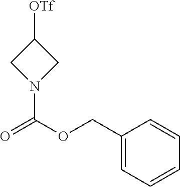 [SMILES: O=C(OCc1ccccc1)N1CC(OS(=O)(=O)C(F)(F)F)C1]

Tf₂O (1.18 g, 4.19 mmol) was added dropwise to a solution of benzyl 3-hydroxyazetidine-1-carboxylate (621 mg, 3 mmol) and TEA (909 mg, 9.0 mmol) in DCM (20 mL) at 0° C. and the reaction stirred for 30 min at 0° C. The mixture was concentrated in vacuo and the residue purified by silica gel column chromatography (PE/EtOAc=3:1) to afford the title compound, 800 mg, 80.0% as a yellow oil.

### Preparation 102

### benzyl 3-((dimethyl(oxo)-λ⁶-sulfaneylidene)amino)azetidine-1-carboxylate

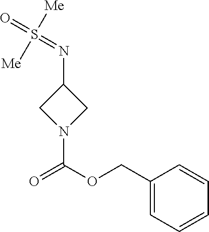 [SMILES: CS(C)(=O)=NC1CN(C(=O)OCc2ccccc2)C1]

A mixture of iminodimethyl-λ⁶-sulfanone (372 mg, 4 mmol) and Cs₂CO₃ (2.60 g, 8 mmol) in DMF (5 mL) was stirred at rt for 2 h. Benzyl 3-(((trifluoromethyl)sulfonyl)oxy)azetidine-1-carboxylate (Preparation 101, 4.07 g, 12 mmol) was added, and the reaction stirred for 18 h at rt. The mixture was concentrated in vacuo and the residue purified by silica gel column chromatography (EtOAc/MeOH=10:1) to afford the title compound (600 mg, 53.6%) as a colorless syrup.

### Preparation 103

### (azetidin-3-ylimino)dimethyl-λ⁶-sulfanone

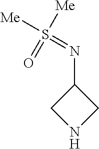

The title compound was obtained as a white waxy solid, 310 mg, crude, from benzyl 3-((dimethyl(oxo)-λ⁶-sulfaneylidene)amino)azetidine-1-carboxylate (Preparation 102), following the procedure described in Preparation 98.

### Preparation 104

### tert-butyl 3-(((trifluoromethyl)thio)methyl)azetidine-1-carboxylate

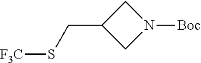 [SMILES: CC(C)(C)OC(=O)N1CC(CSC(F)(F)F)C1]

A mixture of tert-butyl 3-(hydroxymethyl)azetidine-1-carboxylate (300 mg, 1.602 mmol), trifluoro(methyl)-λ⁴-sulfane (1.295 g, 6.41 mmol) and n-Bu₄NI (7.10 g, 19.23 mmol) in toluene (30 mL) was stirred for 10 h at 80° C. The reaction was quenched by the addition of water (1 mL) and the resulting solids were filtered off. The filtrate was extracted with EtOAc (3×20 mL), and the combined organic phases concentrated in vacuo. The residue was purified by silica gel column eluting with EtOAc/PE (3:1) to afford the title compound, 132 mg (30.4%) as a light yellow solid.

### Preparation 105

### tert-butyl 3-(((trifluoromethyl)sulfinyl)methyl)azetidine-1-carboxylate

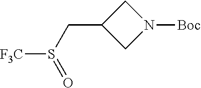 [SMILES: CC(C)(C)OC(=O)N1CC(CS(=O)C(F)(F)F)C1]

tert-Butyl 3-(((trifluoromethyl)thio)methyl)azetidine-1-carboxylate (Preparation 104, 150 mg, 0.553 mmol) and oxone (186 mg, 1.106 mmol) in THF (2 mL), water (2 mL) and EtOH (2 mL) was stirred for 12 h at 0° C. The reaction was quenched by the addition of Na₂S2O₃ (1 mL) and the resulting solids filtered off. The filtrate was extracted with EtOAc, the organic solution concentrated in vacuo and the residue purified by silica gel column with EtOAc/PE (1:5) to give the title compound, 76 mg, 47.8% as a light yellow solid. LCMS m/z=289 [M+H]⁺

### Preparation 106

### 3-(((trifluoromethyl)sulfinyl)methyl)azetidine trifluoroacetate

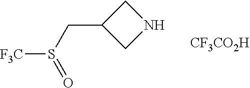 [SMILES: O=S(CC1CNC1)C(F)(F)F ; "CF3CO2H"]

A solution of tert-butyl 3-(((trifluoromethyl)sulfinyl)methyl)azetidine-1-carboxylate (Preparation 105, 100 mg, 0.348 mmol) and TFA (1.00 mL) in DCM (1.00 mL) was stirred for 1 h at rt. The reaction was evaporated under reduced pressure to afford the title compound, 50 mg, 76.7% as a light yellow solid. LCMS m/z=188 [M+H]⁺

### Preparation 107

### (azetidin-3-ylmethyl)dimethylphosphine oxide trifluoroacetate

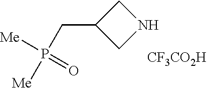

To a solution of (methylphosphonoyl)methane (393 mg, 5.03 mmol) in THF (30 mL) was added NaH (200 mg, 5.04 mmol) and tert-butyl 3-(iodomethyl)azetidine-1-carboxylate (500 mg, 1.68 mmol) and the reaction stirred for 1 h at rt. The reaction was quenched with water and extracted with EtOAc. The organic phase was washed with brine, dried and evaporated under reduced pressure to afford tert-butyl 3-((dimethylphosphoryl)methyl)azetidine-1-carboxylate (1.8 g) as a yellow oil. This was dissolved in DCM (15 mL), trifluoroacetic acid (230 mg, 2.02 mmol) was added and the reaction stirred at rt for 1 h. The resulting solution was evaporated under reduced pressure to give the title compound as a yellow oil, 560 mg.

### Preparation 108

### 8-bromo-3-chloro-5-methoxyisoquinoline

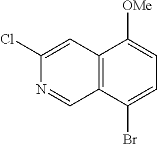 [SMILES: COc1ccc(Br)c2cnc(Cl)cc12]

Br₂ (0.4 mL, 7.75 mmol) was added slowly to a suspension of 3-chloro-5-methoxyisoquinoline (1.0 g, 5.16 mmol) in AcOH (10 mL) at rt and the resulting mixture stirred overnight. Additional Br₂ (0.15 mL) was added and stirring continued for 3 h. The reaction mixture was diluted with EtOAc and slowly quenched with sat. aq. NaHCO₃ and extracted with EtOAc. The combined extracts were dried (Na₂SO₄) and evaporated to dryness in vacuo. The residue was purified by ISCO chromatography (0-70% EtOAc/Hex) to afford the title compound as an orange solid, 1.08 g, 77%. ¹H NMR (400 MHz, DMSO-d₆) δ: 9.26 (d, 1H), 8.05 (s, 1H), 7.94 (d, 1H), 7.24 (d, 1H), 4.02 (s, 3H).

### Preparation 109

### 8-bromo-3-chloroisoquinolin-5-ol

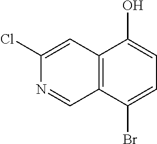 [SMILES: Oc1ccc(Br)c2cnc(Cl)cc12]

BBr₃ (30.8 mL, 30.8 mmol, 1M in DCM) was added to a solution of 8-bromo-3-chloro-5-methoxyisoquinoline (Preparation 108, 2.4 g, 8.81 mmol) in DCM (31.5 mL) at 0° C. and the reaction stirred at rt overnight. The reaction mixture was slowly poured into cold H₂O with rapid stirring and the resulting solids collected via vacuum filtration and dried under high vacuum for 1 h to afford the title compound as a yellow solid, 2.20 g, 97%. ¹H NMR (400 MHz, DMSO-d₆) δ: 9.19 (d, 1H), 8.01 (d, 1H), 7.79 (d, 1H), 7.06 (d, 1H).

### Preparation 110

### 8-bromo-3-chloroisoquinolin-5-yl trifluoromethanesulfonate

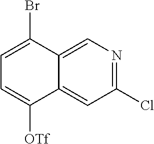 [SMILES: O=S(=O)(Oc1ccc(Br)c2cnc(Cl)cc12)C(F)(F)F]

Tf₂O (2.59 mL, 15.32 mmol) was added dropwise to a solution of TEA (3.2 mL, 23 mmol) and 8-bromo-3-chloroisoquinolin-5-ol (Preparation 109, 1.98 g, 7.66 mmol) in dry DCM (70 mL) at −60° C. The reaction was warmed to rt and stirred for 1 h. The mixture was evaporated to dryness in vacuo and the residue purified by ISCO chromatography (DCM) to afford the title compound as a beige solid (2.51 g, 84%). ¹H NMR (400 MHz, DMSO-d₆) δ: 9.49 (d, 1H), 8.22 (d, 1H), 8.03 (d, 1H), 7.95 (s, 1H).

### Preparation 111

### 8-bromo-3-chloro-5-(prop-1-en-2-yl)isoquinoline

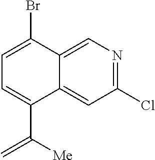

A solution of 4,4,5,5-tetramethyl-2-(prop-1-en-2-yl)-1,3,2-dioxaborolane (3.61 mL, 19.20 mmol), 8-bromo-3-chloroisoquinolin-5-yl trifluoromethanesulfonate (Preparation 110, 7.5 g, 19.2 mmol), K₂CO₃ (2.65 g, 19.2 mmol) and PdCl₂(dppf)·DCM (1.568 g, 1.92 mmol) in dioxane (87 mL) and H₂O (8.7 mL) was purged with N₂ for 5 min then stirred at 45° C. overnight. The mixture was diluted with EtOAc (100 mL) and washed with brine (2×40 mL). The combined organics were dried (Na₂SO₄) and evaporated to dryness in vacuo. The residue was purified using ISCO chromatography (0-10% EtOAc in Hexanes) to afford the title compound as a white solid (2.75 g, 50.6%).

### Preparation 112

### 2-(8-bromo-3-chloroisoquinolin-5-yl)prop-2-en-1-ol

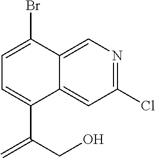 [SMILES: C=C(CO)c1ccc(Br)c2cnc(Cl)cc12]

The title compound was obtained as a white solid, 2.1 g, 54.9% yield, from 8-bromo-3-chloroisoquinolin-5-yl trifluoromethanesulfonate (Preparation 110) and 2-(4,4,5,5-tetramethyl-1,3,2-dioxaborolan-2-yl)prop-2-en-1-ol, following a similar procedure to that described in Preparation 111. LCMS m/z=298 [M+H]⁺

### Preparation 113

### 8-bromo-3-chloro-5-isopropylisoquinoline

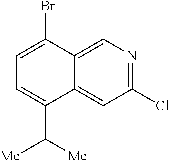 [SMILES: CC(C)c1ccc(Br)c2cnc(Cl)cc12]

A mixture of 8-bromo-3-chloro-5-(prop-1-en-2-yl)isoquinoline (Preparation 112, 500 mg, 1.77 mmol) and PtO₂ (40.2 mg, 0.177 mmol) in EtOAc (12 mL) was placed under a balloon of H₂ and stirred at rt for 1.5 h. The solids were removed by filtration through a pad of Celite® and washed with EtOAc. The combined organics were evaporated to dryness in vacuo and the residue was purified by ISCO chromatography (0-15% EtOAc/Hex) to afford the title compound as a white solid (376 mg, 74.7%). LCMS m/z=286 [M+H]⁺

### Preparation 114

### 2-(8-bromo-3-chloroisoquinolin-5-yl)propan-1-ol

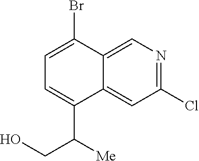 [SMILES: CC(CO)c1ccc(Br)c2cnc(Cl)cc12]

The title compound was obtained as a yellow solid, 1.8 g, 90% yield, from 2-(8-bromo-3-chloroisoquinolin-5-yl)prop-2-en-1-ol (Preparation 112), following a similar procedure to that described in Preparation 113. LCMS m/z=300 [M+H]⁺

### Preparation 115

### 8-(azetidin-1-yl)-3-chloro-5-isopropylisoquinoline

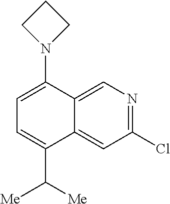 [SMILES: CC(C)c1ccc(N2CCC2)c2cnc(Cl)cc12]

To a solution of 8-bromo-3-chloro-5-isopropylisoquinoline (Preparation 113, 500 mg, 1.75 mmol) in DMSO was added azetidine (199 mg, 3.50 mmol), K₃PO₄ (744 mg, 3.5 mmol), L-proline (201 mg, 1.75 mmol) and CuI (500 mg, 2.62 mmol) under N₂ and the reaction stirred at 80° C. for 2 h. The cooled reaction was diluted with water (100 mL) then extracted with EtOAc (2×30 mL) and the organic layers combined. The organic solution was washed with brine (20 mL) dried over anhydrous Na₂SO₄ and concentrated in vacuo. The crude product was purified by TLC to give the title compound 230 mg, 50.4%, as a yellow solid. LCMS m/z=261 [M+H]⁺

### Preparation 116

### Trans-rac-3-chloro-5-isopropyl-8-(2-methyl-3-(4-methyl-4H-1,2,4-triazol-3-yl)azetidin-1-yl)isoquinoline

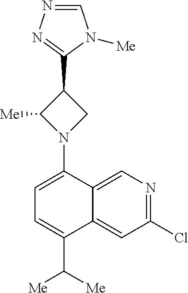 [SMILES: CC(C)c1ccc(N2C[C@H](c3nncn3C)[C@H]2C)c2cnc(Cl)cc12]

The title compound was obtained as a yellow solid, 20 mg, 41% yield, from 8-bromo-3-chloro-5-isopropylisoquinoline (Preparation 113) and trans-rac-4-methyl-3-(2-methylazetidin-3-yl)-4H-1,2,4-triazole trifluoroacetate (Preparation 81), following the procedure described in Preparation 115. LCMS m/z=356 [M+H]⁺

### Preparation 117

### 2-(1-(3-chloro-5-isopropylisoquinolin-8-yl)-3-methylazetidin-3-yl)-1,3,4-oxadiazole

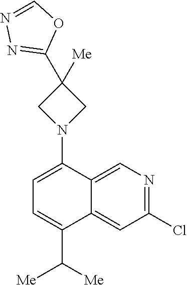 [SMILES: CC(C)c1ccc(N2CC(C)(c3nnco3)C2)c2cnc(Cl)cc12]

The title compound was obtained as a yellow solid, 150 mg, 63% yield, from 8-bromo-3-chloro-5-isopropylisoquinoline (Preparation 113) and 2-(3-methylazetidin-3-yl)-1,3,4-oxadiazole trifluorocetate (Preparation 93), following the procedure described in Preparation 115. LCMS m/z=343 [M+H]⁺

### Preparation 118

### Trans-rac-2-(1-(3-chloro-5-isopropylisoquinolin-8-yl)-2-methylazetidin-3-yl)-1,3,4-oxadiazole

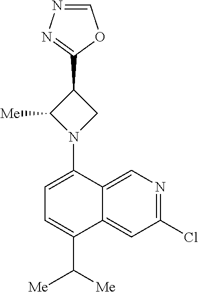 [SMILES: CC(C)c1ccc(N2C[C@H](c3nnco3)[C@H]2C)c2cnc(Cl)cc12]

The title compound was obtained as a yellow solid, 30 mg, 17% yield, from 8-bromo-3-chloro-5-isopropylisoquinoline (Preparation 113) and trans-rac-2-(2-methylazetidin-3-yl)-1,3,4-oxadiazole trifluoroacetate (Preparation 96), following the procedure described in Preparation 115. LCMS m/z=343 [M+H]⁺

### Preparation 119

### 2-(1-(3-chloro-5-isopropylisoquinolin-8-yl)azetidin-3-yl)-1,3,4-oxadiazole

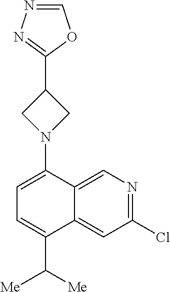 [SMILES: CC(C)c1ccc(N2CC(c3nnco3)C2)c2cnc(Cl)cc12]

A mixture of 8-bromo-3-chloro-5-isopropylisoquinoline (Preparation 113, 80 mg, 0.281 mmol), 2-(azetidin-3-yl)-1,3,4-oxadiazole (42.1 mg, 0.337 mmol), CuI (26.5 mg, 0.140 mmol), L-Proline (3.23 mg, 0.0281 mmol) and K₃PO₄ (178 mg, 0.843 mmol) in DMSO was stirred overnight at 100° C. under N₂. The cooled mixture was partitioned between EtOAc and water, the layers separated and the organic phase dried over Na₂SO₄. The organic layer was concentrated in vacuo and the residue was purified by prep-TLC with PE:EtOAc (5:1) to give the title compound, 60 mg, 65% as a light yellow solid. LCMS m/z=329 [M+H]⁺

### Preparation 120

### tert-butyl (S)-((1-(3-chloro-5-isopropylisoquinolin-8-yl)azetidin-2-yl)methyl)carbamate

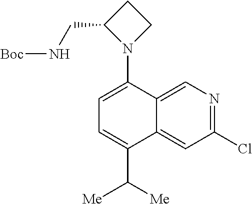 [SMILES: CC(C)c1ccc(N2CC[C@H]2CNC(=O)OC(C)(C)C)c2cnc(Cl)cc12]

A mixture of 8-bromo-3-chloro-5-isopropylisoquinoline (Preparation 113, 200 mg, 0.703 mmol), tert-butyl (S)-(azetidin-2-ylmethyl)carbamate hydrochloride (156 mg, 0.703 mmol), Cs₂CO₃ (456 mg, 1.40 mmol) and Xantphos Pd G2 (124 mg, 0.141 mmol) in dioxane (10 mL) was stirred at 100° C. for 16 h. The reaction mixture was concentrated in vacuo and purified by prep. TLC (PE:EtOAc=4:1), to afford the title compound, 180 mg, 65.7% as a light-yellow solid. LCMS m/z=390 [M+H]⁺

### Preparation 121

### (S)-(1-(3-chloro-5-isopropylisoquinolin-8-yl)azetidin-2-yl)methanol

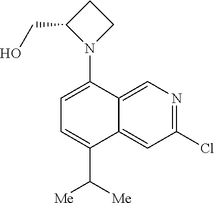 [SMILES: CC(C)c1ccc(N2CC[C@H]2CO)c2cnc(Cl)cc12]

A mixture of 8-bromo-3-chloro-5-isopropylisoquinoline (Preparation 113, 100 mg, 0.351 mmol) Xantphos Pd G2 (31.1 mg, 0.0351 mmol), Xantphos (20.3 mg, 0.0351 mmol), (S)-azetidin-2-ylmethanol and Cs₂CO₃ (228 mg, 0.702 mmol) in DMF (1 mL), was stirred at 90° C. for 16 h. The cooled mixture was concentrated in vacuo and the residue was purified by prep-TLC with DCM/MeOH (10/1) to afford the title compound, as a white solid, 90 mg. LCMS m/z=291 [M+H]⁺

### Preparation 122

### 8-(3-(1H-1,2,3-triazol-1-yl)azetidin-1-yl)-3-chloro-5-isopropylisoquinoline

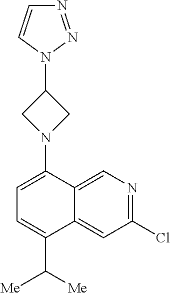 [SMILES: CC(C)c1ccc(N2CC(n3ccnn3)C2)c2cnc(Cl)cc12]

A mixture of 8-bromo-3-chloro-5-isopropylisoquinoline (Preparation 113, 150 mg, 0.527 mmol), 1-(azetidin-3-yl)-1H-1,2,3-triazole (150 mg, 1.20 mmol), Cs₂CO₃ (11.8 g, 36.3 mmol) and XantPhos Pd G2 (106 mg, 0.120 mmol) in dioxane (15 mL) was stirred at 100° C. for 16 h. The reaction mixture was concentrated in vacuo and purified by prep. TLC (DCM:MeOH=40:1), to afford the title compound, 140 mg, 81.2%, as a yellow solid. LCMS m/z=327 [M+H]⁺

### Preparation 123

### (2R,3S)-1-(3-chloro-5-isopropylisoquinolin-8-yl)-2-methylazetidin-3-ol

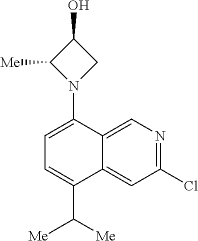 [SMILES: CC(C)c1ccc(N2C[C@H](O)[C@H]2C)c2cnc(Cl)cc12]

Cs₂CO₃ (544 mg, 1.67 mmol) was added to (2R,3S)-2-methylazetidin-3-ol ((1S)-7,7-dimethyl-2-oxobicyclo[2.2.1]heptan-1-yl)methanesulfonate (Preparation 72, 447 mg, 1.40 mmol), 8-bromo-3-chloro-5-isopropylisoquinoline (Preparation 113, 400 mg, 1.40 mmol) and XantPhos Pd G3 (124 mg, 0.140 mmol) in dioxane (15 mL) and the reaction stirred at 85° C. for 4 h under N₂. The mixture was diluted with EtOAc (100 mL) and washed with brine. The organic layer was dried over Na₂SO₄ and concentrated in vacuo. The residue was purified by prep-TLC (DCM:MeOH=30:1) to afford the title compound, 210 mg, 51.5% as a yellow solid. LCMS m/z=291 [M+H]⁺

### Preparation 124

### 2-(1-(3-chloro-5-isopropylisoquinolin-8-yl)azetidin-3-yl)-5-methyl-1,3,4-oxadiazole

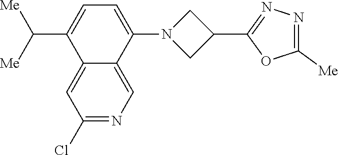 [SMILES: Cc1nnc(C2CN(c3ccc(C(C)C)c4cc(Cl)ncc34)C2)o1]

The title compound was obtained as a yellow oil, from 8-bromo-3-chloro-5-isopropylisoquinoline (Preparation 113) and 2-(azetidin-3-yl)-5-methyl-1,3,4-oxadiazole trifluoroacetate (Preparation 95), following a similar procedure to that described in Preparation 123. LCMS m/z=343 [M+H]⁺

### Preparation 125

### 3-chloro-5-isopropyl-8-(3-(4-methyl-4H-1,2,4-triazol-3-yl)azetidin-1-yl)isoquinoline

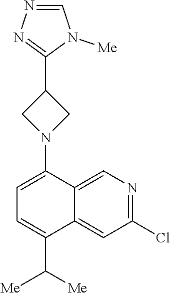 [SMILES: CC(C)c1ccc(N2CC(c3nncn3C)C2)c2cnc(Cl)cc12]

A mixture of 3-(azetidin-3-yl)-4-methyl-1,2,4-triazole (80.0 mg, 0.58 mmol), 8-bromo-3-chloro-5-isopropylisoquinoline (Preparation 113, 164.8 mg, 0.58 mmol), Cs₂CO₃ (378.45 mg, 1.158 mmol) and Xantphos Pd G4 (51.41 mg, 0.058 mmol) in dioxane (5 mL) was stirred for 3 h at 100° C. in a sealed vessel under N₂. The reaction was quenched by the addition of H₂O (2 mL), the solids filtered off and the filtrate extracted with EtOAc (3×5 mL). The combined organic extracts were concentrated in vacuo and the residue was purified by silica gel column with DCM:MeOH (10:1) to give the title compound, 50 mg, 25.3% as a light yellow solid. LCMS m/z=342 [M+H]⁺

### Preparation 126

### 6-(3-chloro-5-isopropylisoquinolin-8-yl)-1-thia-6-azaspiro[3.3]heptane 1,1-dioxide

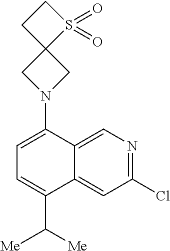 [SMILES: CC(C)c1ccc(N2CC3(CCS3(=O)=O)C2)c2cnc(Cl)cc12]

1-Thia-6-azaspiro[3.3]heptane 1,1-dioxide (25.7 mg, 0.175 mmol), Cs₂CO₃ (221 mg, 0.678 mmol) and XantPhos Pd G4 (32.5 mg, 0.034 mmol) were added to a solution of 8-bromo-3-chloro-5-isopropylisoquinoline (Preparation 113, 96.4 mg, 0.339 mmol) in dioxane and the reaction stirred under N₂ at 100° C. overnight. The cooled mixture was partitioned between EtOAc and water and the layers separated. The organic layer was concentrated in vacuo and was purified by prep-TLC (5% MeOH in DCM) to afford the title compound, 60 mg, 97% yield. LCMS m/z=351 [M+H]⁺

### Preparation 127

### 8-(3-((1H-1,2,3-triazol-1-yl)methyl)azetidin-1-yl)-3-chloro-5-isopropylisoquinoline

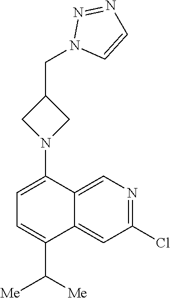 [SMILES: CC(C)c1ccc(N2CC(Cn3ccnn3)C2)c2cnc(Cl)cc12]

Part A: To a stirred solution of tert-butyl 3-((1H-1,2,3-triazol-1-yl)methyl)azetidine-1-carboxylate (Preparation 70, 163 mg, 0.68 mmol) in DCM (1 mL) was added TFA (0.523 mL, 6.84 mmol) and the reaction stirred at rt for 1 h. The reaction was concentrated in vacuo, the residue dissolved in MeOH (6 mL), the solution cooled to 0° C., MP carbonate resin added and the mixture stirred until the pH was 9. The mixture was filtered and the filtrate evaporated under reduced pressure to provide 1-(azetidin-3-ylmethyl)-1H-1,2,3-triazole, as a light yellow oil, 83 mg.

Part B: A solution of 1-(azetidin-3-ylmethyl)-1H-1,2,3-triazole (36.4 mg, 0.264 mmol), 8-bromo-3-chloro-5-isopropylisoquinoline (Preparation 113, 75 mg, 0.264 mmol), Pd₂(dba)₃ (12.07 mg, 0.013 mmol), BINAP (16.41 mg, 0.026 mmol) and Cs₂CO₃ (86 mg, 0.264 mmol) in dioxane (2.20 mL) was degassed with N₂ and stirred at 80° C. for 5 h. Further 1-(azetidin-3-ylmethyl)-1H-1,2,3-triazole was added and the reaction stirred overnight. The reaction was filtered through Celite® and the filtrate concentrated in vacuo. The residue was purified by Isco Combiflash® (0-5% MeOH in DCM) to afford the title compound as a light yellow solid, 59.5 mg. LCMS m/z=342 [M+H]⁺

### Preparation 128

### 3-chloro-5-isopropyl-8-((2R,3S)-2-methyl-3-(1H-1,2,3-triazol-1-yl)azetidin-1-yl)isoquinoline

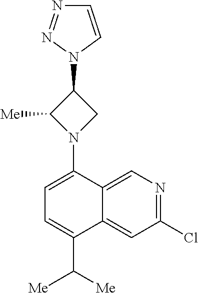 [SMILES: CC(C)c1ccc(N2C[C@H](n3ccnn3)[C@H]2C)c2cnc(Cl)cc12]

A solution of 1-((2R,3S)-2-methylazetidin-3-yl)-1H-1,2,3-triazole (Preparation 76, 29.1 mg, 0.211 mmol), 8-bromo-3-chloro-5-isopropylisoquinoline (Preparation 113, 60 mg, 0.211 mmol), Pd₂(dba)₃ (9.65 mg, 0.0105 mmol), BINAP (13.13 mg, 0.021 mmol) and Cs₂CO₃ (68.7 mg, 0.211 mmol) in dioxane (1.76 mL) was degassed with N₂ and stirred at 80° C. for 4 h. The cooled mixture was filtered through Celite® and the filtrate evaporated under reduced pressure. The crude product was purified by ISCO chromatography (05% MeOH in DCM) to afford the title compound as a light yellow solid, 52.4 mg, 72.7%. LCMS m/z=342 [M+H]⁺

### Preparation 129

### 2-(3-chloro-8-(3-methoxyazetidin-1-yl)isoquinolin-5-yl)propan-1-ol

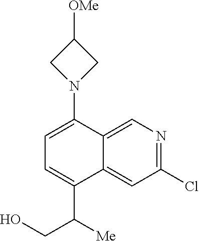 [SMILES: COC1CN(c2ccc(C(C)CO)c3cc(Cl)ncc23)C1]

To a solution of 2-(8-bromo-3-chloroisoquinolin-5-yl)propan-1-ol (Preparation 114, 150 mg, 0.499 mmol) in dioxane was added 3-methoxyazetidine (43.4 mg, 0.499 mmol), Cs₂CO₃ (485 mg, 1.49 mmol) and BINAP Pd G3 (49.5 mg, 0.05 mmol) under N₂ and the reaction stirred at 100° C. for 3 h. The cooled mixture was diluted with water (20 mL), extracted with EtOAc (2×20 mL) and the organic layers combined. The resulting mixture was washed with brine (20 mL), dried over anhydrous Na₂SO₄ and concentrated in vacuo. The crude product was purified by prep TLC to give the title compound, 90 mg, 58.8% as a yellow solid. LCMS m/z=307 [M+H]⁺

### Preparation 130

### 2-(3-chloro-8-((R)-2-methylazetidin-1-yl)isoquinolin-5-yl)propan-1-ol

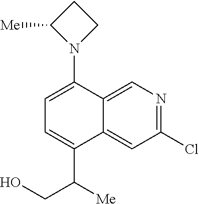 [SMILES: CC(CO)c1ccc(N2CC[C@H]2C)c2cnc(Cl)cc12]

Cs₂CO₃ (180 mg, 0.593 mmol) was added to Brettphos Pd G3 (22.6 mg, 0.023 mmol), 2-(8-bromo-3-chloroisoquinolin-5-yl)propan-1-ol (Preparation 114, 70 mg, 0.233 mmol) and (2R)-2-methylazetidine camphorsulfonate (JOC 2016, 81, 3031-3036, 63.6 mg, 0.233 mmol) in dioxane (10 mL) and the mixture was stirred at 100° C. for 3 h under N₂. The cooled mixture was diluted with EtOAc (100 mL) and washed with brine (50 mL×2), dried over Na₂SO₄ and concentrated in vacuo. The residue was purified by prep-TLC with DCM/MeOH=25: 1 to give the title compound, 40 mg, 59% as a yellow solid. LCMS m/z=291 [M+H]⁺

### Preparation 131

### methyl 3-(3-chloro-5-isopropylisoquinolin-8-yl)cyclobutane-1-carboxylate

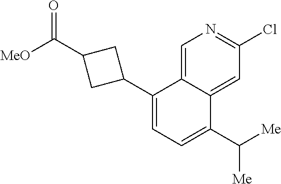 [SMILES: COC(=O)C1CC(c2ccc(C(C)C)c3cc(Cl)ncc23)C1]

A solution of (1s,3s)-methyl 3-iodocyclobutanecarboxylate (291 mg, 1.211 mmol) in DMA (3.1 mL) was purged with N₂, Rieke Zinc in THF (1.46 mL, 1.117 mmol) added and the mixture stirred for 30 mins. 8-Bromo-3-chloro-5-isopropylisoquinoline (Preparation 113, 265 mg, 0.931 mmol), Pd(OAc)₂ (20.91 mg, 0.093 mmol) and cataCXium® A (33.4 mg, 0.093 mmol) were added, the mixture purged again with N₂ and the reaction stirred at rt overnight. The mixture was filtered through Celite®, washing through with EtOAc, and the filtrate washed with water (2×). The organic layer was dried over Na₂SO₄, filtered and concentrated in vacuo. The crude product was purified by Isco Combiflash® (0-15% EtOAc in Hexanes) to give the title compound as a mixture as an orange oil, 200.4 mg, 67.7% yield. LCMS m/z=318 [M+H]⁺

### Preparation 132

### (3-(3-chloro-5-isopropylisoquinolin-8-yl)cyclobutyl)methanol

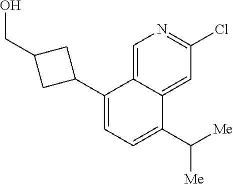 [SMILES: CC(C)c1ccc(C2CC(CO)C2)c2cnc(Cl)cc12]

To a solution of methyl 3-(3-chloro-5-isopropylisoquinolin-8-yl)cyclobutane-1-carboxylate (Preparation 131, 200 mg, 0.629 mmol) in THF (1.2 mL) at 0° C. was added LiAlH₄ (29.9 mg, 0.787 mmol) and the reaction stirred at 0° C. for 30 min. Na₂SO₄ was added until bubbling ceased and the mixture filtered, washing through with EtOAc. The organic solution was concentrated in vacuo and the residue purified by Isco Combiflash® (0-60% EtOAc in Hexanes) to give the title compound, as a yellow oil, 45.5 mg, 25.0% yield. LCMS m/z=290 [M+H]⁺

### Preparation 133

### (3-(3-chloro-5-isopropylisoquinolin-8-yl)cyclobutyl)methyl methanesulfonate

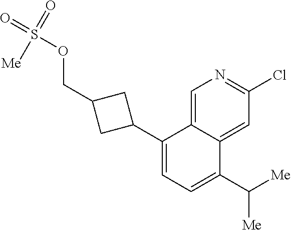 [SMILES: CC(C)c1ccc(C2CC(COS(C)(=O)=O)C2)c2cnc(Cl)cc12]

To a solution of (3-(3-chloro-5-isopropylisoquinolin-8-yl)cyclobutyl)methanol (Preparation 132, 45 mg, 0.155 mmol) and TEA (0.22 mL, 0.155 mmol) in DCM (0.621 mL) was added methanesulfonyl chloride (12.02 μL, 0.155 mmol) and the reaction stirred at rt overnight. The reaction was diluted with DCM and washed with water (2×10 mL). The organic layer was dried over anhydrous Na₂SO₄, filtered and concentrated in vacuo. The residue was purified by Isco Combiflash® (0-65% EtOAc in hexanes) to give the title compounds, as a light yellow oil, 41.4 mg, 72.5% yield. LCMS m/z=368 [M+H]⁺

### Preparation 134

### 3-chloro-5-isopropyl-8-(3-((methylsulfonyl)methyl)cyclobutyl)isoquinoline

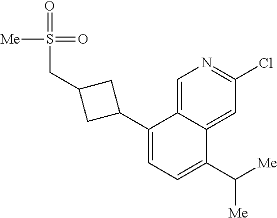 [SMILES: CC(C)c1ccc(C2CC(CS(C)(=O)=O)C2)c2cnc(Cl)cc12]

A solution of (3-(3-chloro-5-isopropylisoquinolin-8-yl)cyclobutyl)methyl methanesulfonate (Preparation 133, 41.5 mg, 0.113 mmol), KI (56.2 mg, 0.338 mmol), and methanesulfinic acid (34.5 mg, 0.338 mmol) in DMF (0.40 mL) was stirred at 100° C. for 2 h. The reaction was cooled to rt, diluted with water and extracted with EtOAc (2×2 mL). The combined organic layers were washed with water (2×2 mL) and brine (2 mL), then dried over Na₂SO₄, filtered and concentrated in vacuo. The residue was purified by Isco Combiflash® (0-10% MeOH in DCM) to give the title compounds, as a colorless oil, 31.2 mg, 79% yield. LCMS m/z=352 [M+H]⁺

### Preparation 135

### tert-butyl 3-(3-chloro-5-(prop-1-en-2-yl)isoquinolin-8-yl)azetidine-1-carboxylate

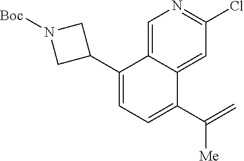 [SMILES: C=C(C)c1ccc(C2CN(C(=O)OC(C)(C)C)C2)c2cnc(Cl)cc12]

Zn (301 mg, 4.60 mmoL) and LiCl (140 mg, 3.3 mmol) was heated under vacuum in a sealed vessel and then flushed with N₂ and cooled. Tert-Butyl 3-iodoazetidine-1-carboxylate (566 mg, 2.0 mmol), 1,2-dibromoethane (23 mg, 0.12 mmol) and THF (2 mL), then chlorotrimethylsilane (4.35 mg, 0.04 mmol) were added and the reaction stirred for 30 mins. This mixture was added to a solution of 8-bromo-3-chloro-5-(prop-1-en-2-yl)isoquinoline (Preparation 111, 150 mg, 0.531 mmol) and Pd(amphos)Cl₂ (19 mg, 0.027 mmol) in DMA (2 mL) under N₂ and the reaction heated to 80° C. for 1 h. The mixture was diluted with EtOAc, washed with water (3×) and brine, then dried over Na₂SO₄. The mixture was filtered, concentrated in vacuo and the crude purified by Isco chromatography to afford the title compound, 92 mg, 48.3% as a reddish foam. LCMS m/z=359 [M+H]⁺

### Preparation 136

### tert-butyl 3-((3-chloro-5-isopropylisoquinolin-8-yl)methyl)azetidine-1-carboxylate

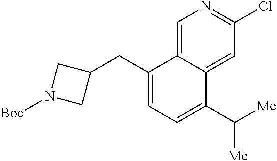 [SMILES: CC(C)c1ccc(CC2CN(C(=O)OC(C)(C)C)C2)c2cnc(Cl)cc12]

The title compound was obtained as a pink oil, 139 mg, 70.3% yield, from 8-bromo-3-chloro-5-isopropylisoquinoline (Preparation 113) and tert-butyl 3-(iodomethyl)azetidine-1-carboxylate following a similar procedure to that described in Preparation 135.

### Preparation 137

### tert-butyl 3-(3-chloro-5-isopropylisoquinolin-8-yl)azetidine-1-carboxylate

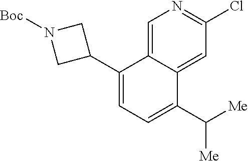 [SMILES: CC(C)c1ccc(C2CN(C(=O)OC(C)(C)C)C2)c2cnc(Cl)cc12]

PtO₂ (5.82 mg) was added to a solution of tert-butyl 3-(3-chloro-5-(prop-1-en-2-yl)isoquinolin-8-yl)azetidine-1-carboxylate (Preparation 135, 92 mg, 0.256 mmol) in EtOAc (2.5 mL) and the reaction stirred at rt under an atmosphere of H₂ overnight. The mixture was filtered, the solvent evaporated under reduced pressure and the residue reacted again under the same conditions for 2 h. The mixture was filtered and the solvent evaporated under reduced pressure to give the title compound, 89 mg, as an off-white foam. LCMS m/z=361 [M+H]⁺

### Preparation 138

### 8-(azetidin-3-yl)-3-chloro-5-isopropylisoquinoline

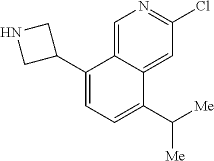 [SMILES: CC(C)c1ccc(C2CNC2)c2cnc(Cl)cc12]

TFA (0.19 mL) was added to a solution of tert-butyl 3-(3-chloro-5-isopropylisoquinolin-8-yl)azetidine-1-carboxylate (Preparation 137) in DCM (1.5 mL) and the reaction stirred for 1 h. The solution was concentrated in vacuo and the residue then partitioned between DCM and aq. NaHCO₃ solution. The organic layer was dried over Na₂SO₄, filtered and evaporated under reduced pressure to give the title compound, 61 mg as an off-white foam. LCMS m/z=261 [M+H]⁺

### Preparation 139

### 8-(azetidin-3-ylmethyl)-3-chloro-5-isopropylisoquinoline

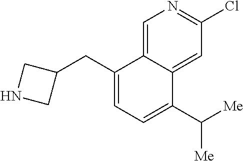 [SMILES: CC(C)c1ccc(CC2CNC2)c2cnc(Cl)cc12]

The title compound was obtained as a viscous oil, 104 mg, crude, from tert-butyl 3-((3-chloro-5-isopropylisoquinolin-8-yl)methyl)azetidine-1-carboxylate (Preparation 136) following a similar procedure to that described in Preparation 138. LCMS m/z=275 [M+H]⁺

### Preparation 140

### 3-chloro-5-isopropyl-8-(1-(methylsulfonyl)azetidin-3-yl)isoquinoline

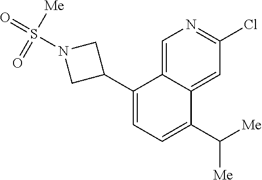 [SMILES: CC(C)c1ccc(C2CN(S(C)(=O)=O)C2)c2cnc(Cl)cc12]

Mesyl chloride (10.76 μL, 0.138 mmol) was added to a solution of 8-(azetidin-3-yl)-3-chloro-5-isopropylisoquinoline (Preparation 138, 30 mg, 0.115 mmol) and TEA (24 μL, 0.173 mmol) in DCM (1 mL) and the reaction stirred at rt for 1 h. The mixture was diluted with DCM, washed with water and dried over Na₂SO₄. The mixture was filtered, concentrated in vacuo and the crude product purified by Isco chromatography (0 to 60% EtOAc/Hex) to give the title compound, 26 mg, 66.7% as a white foam. LCMS m/z=339 [M+H]⁺

### Preparation 141

### 1-(3-(3-chloro-5-isopropylisoquinolin-8-yl)azetidin-1-yl)ethan-1-one

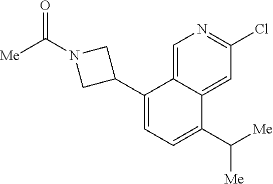 [SMILES: CC(=O)N1CC(c2ccc(C(C)C)c3cc(Cl)ncc23)C1]

Acetyl chloride (18 μL, 0.257 mmol) was added to a solution of 8-(azetidin-3-yl)-3-chloro-5-isopropylisoquinoline (Preparation 138, 61 mg, 0.234 mmol) and TEA (49 μL, 0.351 mmol) in DCM (1.5 mL) and the reaction stirred at rt overnight. The mixture was diluted with DCM and washed with water. The organic phase was dried over Na₂SO₄, filtered and evaporated under reduced pressure. The crude product was purified by Isco chromatography (0 to 10% MeOH/DCM) to give the title compound, 27 mg as a white foam. LCMS m/z=303 [M+H]⁺

### Preparation 142

### 1-(3-((3-chloro-5-isopropylisoquinolin-8-yl)methyl)azetidin-1-yl)ethan-1-one

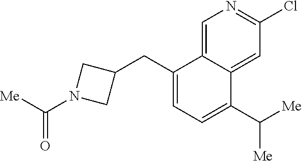 [SMILES: CC(=O)N1CC(Cc2ccc(C(C)C)c3cc(Cl)ncc23)C1]

The title compound was obtained as an off-white foam, 115 mg, from 8-(azetidin-3-ylmethyl)-3-chloro-5-isopropylisoquinoline (Preparation 139) and acetyl chloride, following a similar procedure to that described in Preparation 141. LCMS m/z=317 [M+H]⁺

### Preparation 143

### 1-((2-(trimethylsilyl)ethoxy)methyl)-1H-1,2,4-triazole

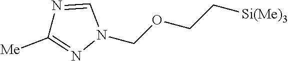

To a solution of 3-methyl-1H-1,2,4-triazole (415 mg, 5 mmol) in THF (10 mL) was added NaH (220 mg, 5.50 mmol, 60%) at 0° C. and the mixture stirred for 1 h. (2-(Chloromethoxy)ethyl)trimethylsilane (1 g, 6.00 mmol) was added and the reaction stirred for 2 h at rt. The reaction was quenched with brine (50 mL), the layers separated and the aqueous layer was extracted with EtOAc (10 mL×3). The combined organic extracts were concentrated in vacuo and the crude product was purified by silica gel column chromatography (PE/EtOAc=1:1) to give the title compound, (600 mg, 56.6%) as a colorless liquid.

### Preparation 144

### 3-chloro-5-isopropyl-8-(3-methyl-1-((2-(trimethylsilyl)ethoxy)methyl)-1H-1,2,4-triazol-5-yl)isoquinoline

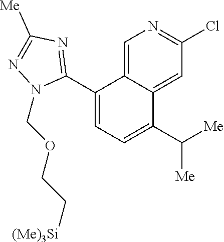

A mixture of 3-methyl-1-((2-(trimethylsilyl)ethoxy)methyl)-1H-1,2,4-triazole (Preparation 143, 106 mg, 0.50 mmol), 8-bromo-3-chloro-5-isopropylisoquinoline (Preparation 113, 142 mg, 0.5 mmol), CuI (104 mg, 0.55 mmol), Pd(OAc)₂ (22.3 mg, 0.1 mmol), PtBu₃HBF₄ (14.4 mg, 0.05 mmol) and Cs₂CO₃ (326 mg, 1.0 mmol) in dioxane (6 mL) was heated at 130° C. for 18 h. The cooled mixture was filtered and the filtrate was concentrated in vacuo. The crude product was purified by prep-TLC (PE/EtOAc=3:1) to afford the title compound, 50 mg, 24.0%, as a light yellow syrup. LCMS m/z=417 [M+H]⁺

### Preparation 145

### 6-chloro-4-iodo-2,7-naphthyridin-1(2H)-one

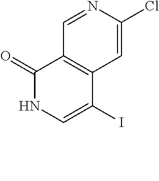

To a solution of 6-chloro-1,2-dihydro-2,7-naphthyridin-1-one (50 g, 0.276 mol) in DMF (300 mL), NIS (74 g, 0.33 mol) was added at 0° C. and the mixture stirred overnight at rt. The reaction mixture was filtered and the filter cake was washed with water and dried under vacuum to afford the title compound (60 g, 70%) as a light-yellow solid. LCMS m/z=307 [M+H]⁺. ¹H NMR (300 MHz, DMSO-d₆) δ: 12.0 (s, 1H), 9.02 (s, 1H), 7.89 (d, 1H), 7.44 (s, 1H).

### Preparation 146

### 1,6-dichloro-4-iodo-2,7-naphthyridine

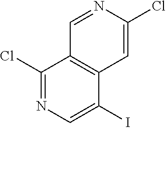

A mixture of 6-chloro-4-iodo-2,7-naphthyridin-1(2H)-one (Preparation 145, 60 g, 0.196 mol) in POCl₃ (320 mL) was stirred at 100° C. for 1.5 h. The mixture was concentrated and neutralized with cooled saturated aq. NaHCO₃. The mixture was extracted with EtOAc (3×300 mL), the combined organic layers dried over Na₂SO₄, filtered and evaporated under reduced pressure to give the title compound, 53 g (84%) as a yellow solid. LCMS m/z=325 [M+H]⁺.

### Preparation 147

### 1,6-dichloro-4-(prop-1-en-2-yl)-1,2-dihydro-2,7-naphthyridine

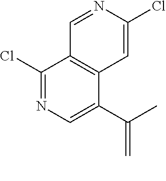

To a solution of 1,6-dichloro-4-iodo-2,7-naphthyridine (Preparation 146, 30 g, 92.5 mmol) in dioxane/H₂O (300/70 mL) was added 4,4,5,5-tetramethyl-2-(prop-1-en-2-yl)-1,3,2-dioxaborolane (15 g, 93 mmol), K₂CO₃ (37.8 g, 276 mmol) and Pd(amphos)Cl₂ (3 g, 4.2 mmol) and the solution was stirred for 0.5 h at 50° C. The mixture was cooled to rt, diluted with water (200 mL) and extracted with EtOAc (2×300 mL). The combined organic layers were washed with brine (200 mL), dried over anhydrous Na₂SO₄ and concentrated in vacuo. The crude product was purified by silica gel column chromatography eluting with EtOAc: PE (1: 10) to give the title compound, 15 g, 68.1% as white solid. LCMS m/z=239 [M+H]⁺.

### Preparation 148

### 1,6-dichloro-4-isopropyl-2,7-naphthyridine

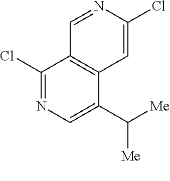

To a solution of 1,6-dichloro-4-(prop-1-en-2-yl)-2,7-naphthyridine (Preparation 147, 4 g, 16.8 mmol) in EtOAc (300 mL) was added PtO₂ (5 g, 22 mmol) and the resulting mixture was stirred at 25° C. for 24 h under H₂ atmosphere. The solid was filtered off and the filtrate was concentrated in vacuo. The residue was purified by silica gel column chromatography (EtOAc:PE, 1:8) to give the title compound, 3 g, 75% as a white solid. LCMS m/z=241 [M+H]⁺. ¹H NMR (300 MHz, DMSO-d₆) δ 9.47 (d, 1H), 8.47 (d, 1H), 8.26 (d, 1H), 3.64 (p, 1H), 1.33 (d, 6H).

### Preparation 149

### 1-(azetidin-1-yl)-4-bromo-6-chloro-2,7-naphthyridine

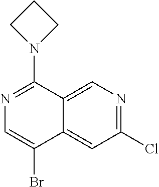 [SMILES: Clc1cc2c(Br)cnc(N3CCC3)c2cn1]

A solution of 4-bromo-1,6-dichloro-2,7-naphthyridine (2.78 g, 10 mmol), azetidine (628 mg, 11 mmol) and TEA (2.02 g, 20 mmol) in IPA (20 mL) was stirred at 100° C. for 2 h. The mixture was filtered, the solid washed with IPA, and dried in vacuo to afford the title compound, 2.7 g, 90.8% yield, as a yellow solid. LCMS m/z=297 [M+H]⁺

### Preparation 150

### 4-bromo-6-chloro-1-(pyrrolidin-1-yl)-2,7-naphthyridine

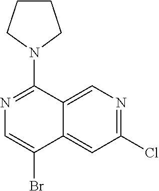 [SMILES: Clc1cc2c(Br)cnc(N3CCCC3)c2cn1]

A mixture of 4-bromo-1,6-dichloro-2,7-naphthyridine (100 mg, 0.359 mmol), pyrrolidine (25.5 mg, 0.359 mmol) and TEA (108 mg, 1.07 mmol) in IPA (50 mL) was stirred for 3 h at 100° C. The cooled reaction was quenched with water and extracted with EtOAc. The organic layers were combined, concentrated in vacuo and purified by silica gel column eluting with PE/EtOAc (1:1) to give the title compound as a yellow solid, 98.0 mg, 87.2% yield. LCMS m/z=314 [M+H]⁺

### Preparation 151

### tert-butyl 1-(4-bromo-6-chloro-2,7-naphthyridin-1-yl)-1,6-diazaspiro[3.3]heptane-6-carboxylate

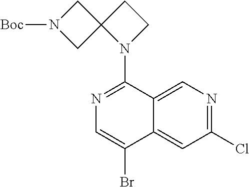 [SMILES: CC(C)(C)OC(=O)N1CC2(CCN2c2ncc(Br)c3cc(Cl)ncc23)C1]

The title compound was obtained as a light yellow solid, 400 mg, 70% yield, from 4-bromo-1,6-dichloro-2,7-naphthyridine and tert-butyl 1,6-diazaspiro[3.3]heptane-6-carboxylate, following a similar procedure to that described in Preparation 150. LCMS m/z=441 [M+H]⁺

### Preparation 152 (R)-4-bromo-6-chloro-1-(2-methylazetidin-1-yl)-2,7-naphthyridine

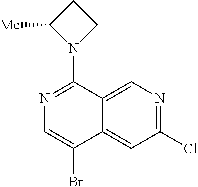 [SMILES: C[C@@H]1CCN1c1ncc(Br)c2cc(Cl)ncc12]

A solution of 4-bromo-1,6-dichloro-2,7-naphthyridine (1.8 g, 6.48 mmol), (2R)-2-methylazetidine camphorsulfonate (JOC 2016, 81, 3031-3036, 2.16 g, 7.12 mmol) and DIPEA (2.83 mL, 16.19 mmol) in IPA (20 mL) was stirred at 90° C. for 1 h under N₂. The mixture was concentrated in vacuo, the residue triturated with water, filtered and the solid dried in vacuo to afford the title compound, 1.81 g, 89.3%, as a tan colored solid. LCMS m/z=313 [M+H]⁺

### Preparation 153

### 1-(azetidin-1-yl)-6-chloro-4-(prop-1-en-2-yl)-2,7-naphthyridine

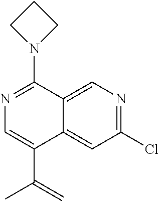 [SMILES: C=C(C)c1cnc(N2CCC2)c2cnc(Cl)cc12]

1-(Azetidin-1-yl)-4-bromo-6-chloro-2,7-naphthyridine (Preparation 149, 2.39 g, 8 mmol), 4,4,5,5-tetramethyl-2-(prop-1-en-2-yl)-1,3,2-dioxaborolane (1.61 g, 9.6 mmol), Pd(dppf)Cl₂ (585 mg, 0.80 mmol), and K₂CO₃ (1.63 g, 12 mmol) were dissolved in dioxane-H₂O (4:1) (50 mL) and the reaction stirred at 80° C. for 4 h. The cooled mixture was concentrated in vacuo and the crude product was purified by silica gel column eluting with MeOH-DCM (2:1) to give the title compound, 1.8 g, 86.5%, as a yellow solid. LCMS m/z=260 [M+H]⁺

### Preparation 154

### 6-chloro-4-(prop-1-en-2-yl)-1-(pyrrolidin-1-yl)-2,7-naphthyridine

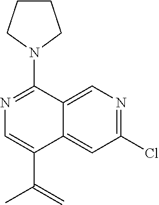 [SMILES: C=C(C)c1cnc(N2CCCC2)c2cnc(Cl)cc12]

4,4,5,5-Tetramethyl-2-(prop-1-en-2-yl)-1,3,2-dioxaborolane (110 mg, 0.655 mmol), Pd(dppf)Cl₂ (51.4 mg, 0.066 mmol) and K₂CO₃ (270 mg, 1.96 mmol) were added to a solution 4-bromo-6-chloro-1-(pyrrolidin-1-yl)-2,7-naphthyridine (Preparation 150, 205.9 mg, 0.655 mmol) in dioxane (20 mL) and the reaction heated to 30° C. and stirred for 3 h under N₂. The reaction was quenched by the addition of water and extracted with EtOAc. The organic layers were combined, concentrated in vacuo and the residue purified by silica gel column eluting with PE/EtOAc (1:1) to give the title compound as a yellow solid, 132 mg, 73.5%. LCMS m/z=274 [M+H]⁺

### Preparation 155

### tert-butyl 1-(6-chloro-4-(prop-1-en-2-yl)-2,7-naphthyridin-1-yl)-1,6-diazaspiro[3.3]heptane-6-carboxylate

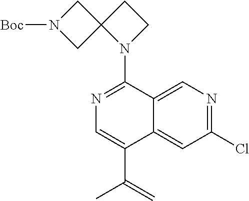 [SMILES: C=C(C)c1cnc(N2CCC23CN(C(=O)OC(C)(C)C)C3)c2cnc(Cl)cc12]

The title compound was obtained as a yellow solid, 150 mg, 69%, from tert-butyl 1-(4-bromo-6-chloro-2,7-naphthyridin-1-yl)-1,6-diazaspiro[3.3]heptane-6-carboxylate (Preparation 151) and 4,4,5,5-tetramethyl-2-(prop-1-en-2-yl)-1,3,2-dioxaborolane following a similar procedure to that described in Preparation 154. LCMS m/z=401 [M+H]⁺

### Preparation 156

### 1-(azetidin-1-yl)-6-chloro-4-isopropyl-2,7-naphthyridine

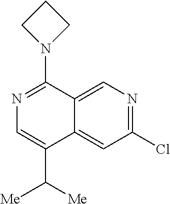 [SMILES: CC(C)c1cnc(N2CCC2)c2cnc(Cl)cc12]

PtO₂ (1.57 g, 2.68 mmol) was added to a solution of 1-(azetidin-1-yl)-6-chloro-4-(prop-1-en-2-yl)-2,7-naphthyridine (Preparation 153, 1.8 g, 6.92 mmol) in EtOAc (200 mL) and the reaction stirred under H₂ at 25° C. for 24 h. The reaction mixture was filtered through Celite®, the filtrate concentrated in vacuo and the product purified by column chromatography (PE-EtOAc (1:5)) to provide the title compound, 1.7 g. 93.8% as a light yellow solid. LCMS m/z=262 [M+H]⁺

### Preparation 157

### 6-chloro-4-isopropyl-1-(pyrrolidin-1-yl)-2,7-naphthyridine

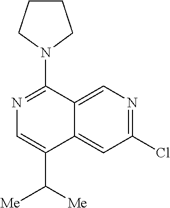 [SMILES: CC(C)c1cnc(N2CCCC2)c2cnc(Cl)cc12]

PtO₂ (99.4 mg, 0.438 mmol) was added to a solution of 6-chloro-4-(prop-1-en-2-yl)-1-(pyrrolidin-1-yl)-2,7-naphthyridine (Preparation 154, 120 mg, 0.438 mmol) in EtOAc (10 mL) and the reaction stirred for 3 h under H₂. The reaction was quenched with water and extracted with EtOAc. The organic layers were combined and evaporated under reduced pressure to afford the title compound as a yellow solid, 113 mg, 93.5%. LCMS m/z=276 [M+H]⁺

### Preparation 158

### tert-butyl 1-(6-chloro-4-isopropyl-2,7-naphthyridin-1-yl)-1,6-diazaspiro[3.3]heptane-6-carboxylate

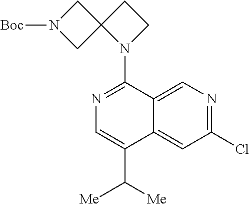 [SMILES: CC(C)c1cnc(N2CCC23CN(C(=O)OC(C)(C)C)C3)c2cnc(Cl)cc12]

The title compound was obtained, 270 mg, 90% as a brown solid, from tert-butyl 1-(6-chloro-4-(prop-1-en-2-yl)-2,7-naphthyridin-1-yl)-1,6-diazaspiro[3.3]heptane-6-carboxylate (Preparation 155), following a similar procedure to that described in Preparation 157. LCMS m/z=403 [M+H]⁺

### Preparation 159

### 1-(6-chloro-4-isopropyl-2,7-naphthyridin-1-yl)azetidine-3-carbonitrile

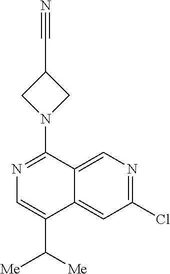 [SMILES: CC(C)c1cnc(N2CC(C#N)C2)c2cnc(Cl)cc12]

A mixture of azetidine-3-carbonitrile hydrochloride (10 mg, 0.084 mmol), 1,6-dichloro-4-isopropyl-2,7-naphthyridine (Preparation 148, 16.27 mg, 0.067 mmol) and TEA (25.60 mg, 0.253 mmol) in IPA (1 mL) was irradiated with microwave radiation for 3 h at 100° C. After cooling to rt, the solids were filtered off and the filtrate evaporated under reduced pressure to give the title compound as a light yellow solid, 12 mg, 49.6%. LCMS m/z=287 [M+H]⁺

### Preparation 160

### 1-(6-chloro-4-isopropyl-2,7-naphthyridin-1-yl)-N,N-dimethylazetidine-3-carboxamide

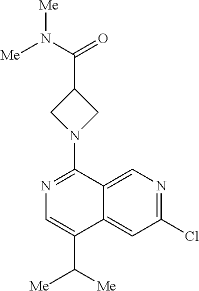 [SMILES: CC(C)c1cnc(N2CC(C(=O)N(C)C)C2)c2cnc(Cl)cc12]

To a solution of N,N-dimethylazetidine-3-carboxamide hydrochloride (81.6 mg, 0.496 mmol) in IPA (5 mL) were added 1,6-dichloro-4-isopropyl-2,7-naphthyridine (Preparation 148, 100 mg, 0.414 mmol) and TEA (125 mg, 1.235 mmol) and the reaction stirred at 100° C. overnight. The cooled mixture was concentrated in vacuo, and the residue purified by prep-TLC (5% MeOH in DCM) to give the title compound, 130 mg, 78.7%. LCMS m/z=333 [M+H]⁺

### Preparations 161 to 167

The compounds in the following table were prepared from 1,6-dichloro-4-isopropyl-2,7-naphthyridine (Preparation 148) and the appropriate secondary amine, following a similar procedure to that described in Preparation 160.

| Prep No | Name, Structure, Amine, Data |
|---|---|
| 161 | 1-(6-chloro-4-isopropyl- |
| | 2,7-naphthyridin-1-yl)azet- |
| | idin-3-ol |
| | 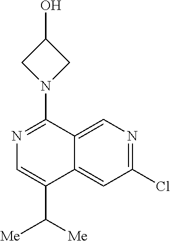 [SMILES: CC(C)c1cnc(N2CC(O)C2)c2cnc(Cl)cc12] |
| | Amine: Azetidin-3-ol |
| | 120 mg, 69.7% yield as a |
| | light yellow solid, LCMS |
| | m/z = 278 [M + H]⁺ |
| 162 | (1-(6-chloro-4-isopropyl- |
| | 2,7-naphthyridin-1-yl)azet- |
| | idin-3-yl)methanol |
| | 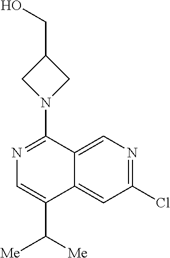 [SMILES: CC(C)c1cnc(N2CC(CO)C2)c2cnc(Cl)cc12] |
| | Amine: (azetidin-3-yl)- |
| | methanol |
| | 108 mg, 74.4% yield as a |
| | light yellow solid, LCMS |
| | m/z = 292 [M + H]⁺ |
| 163 | N-((1-(6-chloro-4-isopropyl-2,7-naphthyr- |
| | idin-1-yl)azetidin-2-yl)methyl)acetamide |
| | 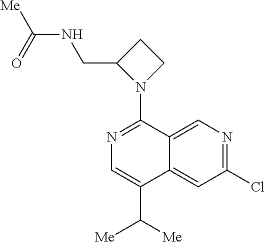 [SMILES: CC(=O)NCC1CCN1c1ncc(C(C)C)c2cc(Cl)ncc12] |
| | Amine: N-((azetidin-2-yl)methyl)acetamide |
| | 110 mg, 42.4% yield, as a yellow solid. |
| | LCMS m/z = 333 [M + H]⁺ |
| 164 | ((1-(6-chloro-4-isopropyl- |
| | 2,7-naphthyridin-1-yl)azet- |
| | idin-3-yl)methyl)dimethyl- |
| | phosphine oxide |
| | 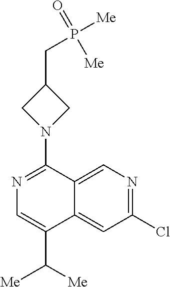 [SMILES: CC(C)c1cnc(N2CC(CP(C)(C)=O)C2)c2cnc(Cl)cc12] |
| | Amine: (azetidin-3-ylmethyl) |
| | dimethylphosphine oxide |
| | trifluoroacetate |
| | (Preparation 107) |
| | 50 mg, 10.4% yield, as a |
| | yellow solid. LCMS m/z = |
| | 352 [M + H]⁺ |
| 165 | 1-(3-(1H-1,2,3-triazol-1-yl)- |
| | azetidin-1-yl)-6-chloro-4- |
| | isopropyl-2,7-naphthyridine |
| | 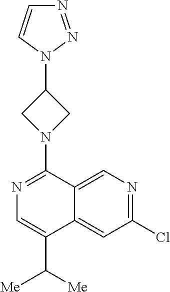 [SMILES: CC(C)c1cnc(N2CC(n3ccnn3)C2)c2cnc(Cl)cc12] |
| | Amine: 1-(azetidin-3-yl)-1H- |
| | 1,2,3-triazole |
| | White solid, 90 mg, 66.1% |
| | yield. LCMS m/z = 329 |
| | [M + H]⁺ |
| 166 | 1-(1-(6-chloro-4-isopropyl-2,7- |
| | naphthyridin-1-yl)azetidin-3- |
| | yl)pyrrolidin-2-one |
| | 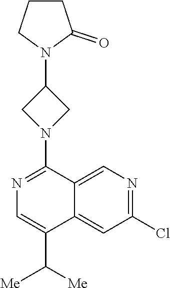 [SMILES: CC(C)c1cnc(N2CC(N3CCCC3=O)C2)c2cnc(Cl)cc12] |
| | Amine: 1-(azetidin-3-yl)- |
| | pyrrolidin-2-one |
| | 80 mg, 56% yield. LCMS |
| | m/z = 345 [M + H]⁺ |
| 167 | 6-chloro-4-isopropyl-1-(1- |
| | methyl-4,6-dihydropyrrolo |
| | [3,4-c]pyrazol-5(1H)-yl)- |
| | 2,7-naphthyridine |
| | 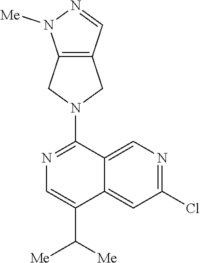 [SMILES: CC(C)c1cnc(N2Cc3cnn(C)c3C2)c2cnc(Cl)cc12] |
| | Amine: 1-methyl-1,4,5,6-tetra- |
| | hydropyrrolo[3,4-c]pyrazole |
| | 90 mg, 66.3% yield. LCMS |
| | m/z = 328 [M + H]⁺ |

### Preparation 168

### 1-(azetidin-1-yl)-6-chloro-4-isopropyl-2,7-naphthyridine

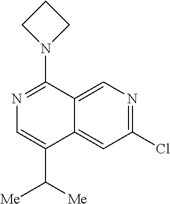 [SMILES: CC(C)c1cnc(N2CCC2)c2cnc(Cl)cc12]

A mixture of azetidine (54.3 mg, 0.581 mmol), 1,6-dichloro-4-isopropyl-2,7-naphthyridine (Preparation 148, 70 mg, 0.29 mmol) and TEA (88.1 mg, 0.871 mmol) in butan-2-ol (2 mL) was stirred at 80° C. for 12 h under N₂. The cooled reaction mixture was concentrated in vacuo and the residue purified using HPLC-14, to give the title compound, 40 mg, 52.6% yield, as a yellow solid.

### Preparation 169

### (S)-1-(1-(6-chloro-4-isopropyl-2,7-naphthyridin-1-yl)pyrrolidin-2-yl)-N,N-dimethylmethanamine

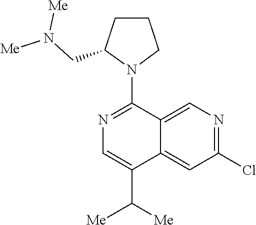 [SMILES: CC(C)c1cnc(N2CCC[C@H]2CN(C)C)c2cnc(Cl)cc12]

The title compound was obtained as a yellow solid, 40 mg, 36.2%, from 1,6-dichloro-4-isopropyl-2,7-naphthyridine (Preparation 148) and N,N-dimethyl-1-((2S)-pyrrolidin-2-yl)methanamine hydrochloride, following a similar procedure to that described in Preparation 168.

### Preparation 170

### 6-chloro-4-isopropyl-1-(2-methylazetidin-1-yl)-2,7-naphthyridine

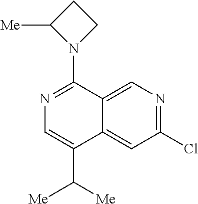 [SMILES: CC(C)c1cnc(N2CCC2C)c2cnc(Cl)cc12]

A mixture of 2-methylazetidine (44.2 mg, 0.622 mmol), 1,6-dichloro-4-isopropyl-2,7-naphthyridine (Preparation 148, 150 mg, 0.622 mmol) and TEA (188 mg, 1.86 mmol) in IPA (3 mL) was stirred at 100° C. for 4 h. The solution was concentrated in vacuo and the residue was purified by prep-TLC (PE:EtOAc, 8:1) to give the title compound, 95 mg, 55.5% as a light yellow solid. LCMS m/z=276 [M+H]⁺

### Preparations 171 to 177

The compounds in the following table were prepared from 1,6-dichloro-4-isopropyl-2,7-naphthyridine (Preparation 148) and the appropriate cyclic amine, following a similar procedure to that described in Preparation 170.

| Prep No | Name, Structure, Amine, Data |
|---|---|
| 171 | tert-butyl (S)-((1-(6-chloro-4-isopropyl-2,7-naphthyridin-1-yl)azetidin-2- |
| | yl)methyl)carbamate |
| | 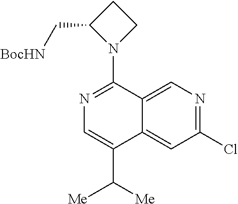 [SMILES: CC(C)c1cnc(N2CC[C@H]2CNC(=O)OC(C)(C)C)c2cnc(Cl)cc12] |
| | Amine: tert-butyl (S)-(azetidin-2-ylmethyl)carbamate |
| | light yellow solid, 200 mg. LCMS m/z = 391 [M + H]⁺ |
| 172 | (S)-6-chloro-1-(2-(fluoromethyl)azetidin-1-yl)-4-isopropyl-2,7-naphthyridine |
| | 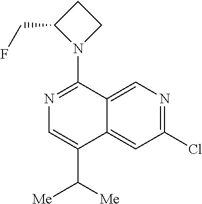 [SMILES: CC(C)c1cnc(N2CC[C@H]2CF)c2cnc(Cl)cc12] |
| | Amine: (2S)-fluoromethylazetidine |
| | light yellow solid, 160 mg, 53.9%. LCMS m/z = 294 [M + H]⁺ |
| 173 | 1-(6-chloro-4-isopropyl-2,7-naphthyridin-1-yl)-6-oxa-1-azaspiro[3.3]heptane |
| | 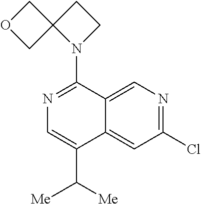 [SMILES: CC(C)c1cnc(N2CCC23COC3)c2cnc(Cl)cc12] |
| | Amine: 6-oxa-1-azaspiro[3.3]heptane |
| | light yellow solid, 80 mg, 70%. LCMS m/z = 304 [M + H]⁺ |
| 174 | 6-(6-chloro-4-isopropyl-2,7-naphthyridin-1-yl)-1-oxa-6-azaspiro[3.3]heptane |
| | 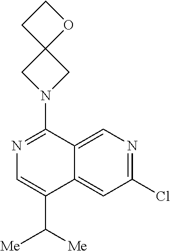 [SMILES: CC(C)c1cnc(N2CC3(CCO3)C2)c2cnc(Cl)cc12] |
| | Amine: 1-oxa-6-azaspiro[3.3]heptane |
| | light yellow solid, 70 mg, 92.5%. LCMS m/z = 304 [M + H]⁺ |
| 175 | 2-(6-chloro-4-isopropyl-2,7-naphthyridin-1-yl)-6-methyl-2,6- |
| | diazaspiro[3.4]octan-5-one |
| | 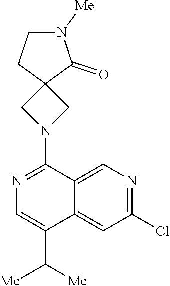 [SMILES: CC(C)c1cnc(N2CC3(CCN(C)C3=O)C2)c2cnc(Cl)cc12] |
| | Amine: 6-methyl-2,6-diazaspiro[3.4]octan-5-one |
| | Light yellow solid, 100 mg, 46.6% yield. LCMS m/z = 345 [M + H]⁺ |
| 176 | 1-(3-(1H-1,2,4-triazol-1-yl)azetidin-1-yl)-6-chloro-4-isopropyl-2,7-naphthyridine |
| | 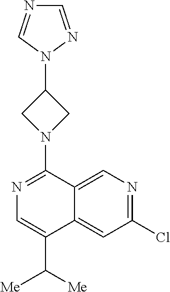 [SMILES: CC(C)c1cnc(N2CC(n3cncn3)C2)c2cnc(Cl)cc12] |
| | Amine: 1-(azetidin-3-yl)-1H-1,2,4-triazole |
| | Light yellow solid, 70 mg, 51.4% yield. LCMS m/z = 329 [M + H]⁺ |
| 177 | 6-chloro-1-(3,3-difluoropyrrolidin-1-yl)-4-isopropyl-2,7-naphthyridine |
| | 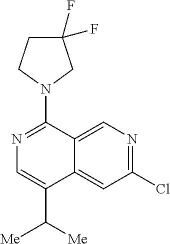 [SMILES: CC(C)c1cnc(N2CCC(F)(F)C2)c2cnc(Cl)cc12] |
| | Amine: 3,3-difluoropyrrolidine |
| | light yellow solid, 30 mg, 41.4%. LCMS m/z = 312 [M + H]⁺ |

### Preparation 178

### 1-(6-chloro-4-isopropyl-2,7-naphthyridin-1-yl)-2-methylazetidin-3-ol

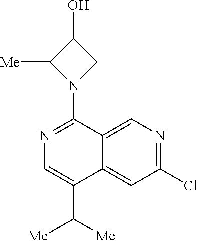 [SMILES: CC(C)c1cnc(N2CC(O)C2C)c2cnc(Cl)cc12]

A mixture of 1,6-dichloro-4-isopropyl-2,7-naphthyridine (Preparation 148, 500 mg, 2.07 mmol), 2-methylazetidin-3-ol trifluoroacetate (630 mg, 7.24 mmol) and TEA (1.04 g, 10.3 mmol) in IPA (7 mL) was heated at 100° C. for 3 h. The cooled reaction mixture was diluted with water (100 mL) and extracted with EtOAc (100 mL×3). The combined organic extracts were washed with brine (100 mL), dried over Na₂SO₄, filtered, concentrated in vacuo and the crude purified by column chromatography (PE:EtOAc=2:1) to afford the title compound, as a light yellow solid, 600 mg, 99%. LCMS m/z=292 [M+H]⁺

### Preparation 179

### (S)-1-(1-(6-chloro-4-isopropyl-2,7-naphthyridin-1-yl)azetidin-2-yl)-N,N-dimethylmethanamine

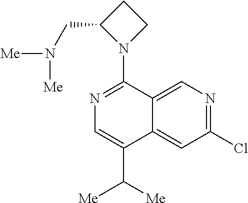 [SMILES: CC(C)c1cnc(N2CC[C@H]2CN(C)C)c2cnc(Cl)cc12]

To a solution of (S)-1-(azetidin-2-yl)-N,N-dimethylmethanamine (100 mg, 0.876 mmol) in IPA was added 1,6-dichloro-4-isopropyl-2,7-naphthyridine (Preparation 148, 234 mg, 0.963 mmol) and TEA (353 mg, 3.50 mmol) and the reaction stirred at 100° C. for 8 h. The cooled solution was diluted with water (100 mL), extracted with EtOAc (2×100 mL) and the organic layers combined. The organic solution was washed with brine (50 mL) dried over anhydrous Na₂SO₄ and concentrated in vacuo. The crude product was purified by silica gel column chromatography (PE:EtOAc, 2: 1) to give the title compound, 100 mg 35.8% as yellow solid. LCMS m/z=319 [M+H]⁺

### Preparations 180 to 190

The compounds in the following table were prepared from 1,6-dichloro-4-isopropyl-2,7-naphthyridine (Preparation 148) and the appropriate cyclic amine, following a similar procedure to that described in Preparation 179.

| Prep no | Name, Structure, Amine, Data |
|---|---|
| 180 | 6-chloro-1-(2-(difluoromethyl)azetidin-1-yl)-4-isopropyl-2,7-naphthyridine |
| | 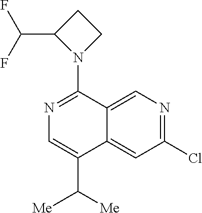 [SMILES: CC(C)c1cnc(N2CCC2C(F)F)c2cnc(Cl)cc12] |
| | Amine: 2-(difluoromethyl)azetidine |
| | yellow solid, 1.2 g, 58%. LCMS m/z = 312 [M + H]⁺ |
| 181 | 6-chloro-4-isopropyl-1-(3-methoxyazetidin-1-yl)-2,7-naphthyridine |
| | 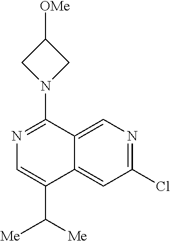 [SMILES: COC1CN(c2ncc(C(C)C)c3cc(Cl)ncc23)C1] |
| | Amine: 3-methoxyazetidine |
| | yellow solid, 110 mg, 91%. LCMS m/z = 292 [M + H]⁺ |
| 182 | 2-(1-(6-chloro-4-isopropyl-2,7-naphthyridin-1-yl)azetidin-3-yl)ethan-1-ol |
| | 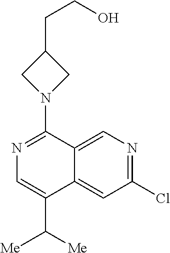 [SMILES: CC(C)c1cnc(N2CC(CCO)C2)c2cnc(Cl)cc12] |
| | Amine: 2-(azetidin-3-yl)ethan-1-ol |
| | white solid, 162 mg, 85.2% |
| 183 | 1-(6-chloro-4-isopropyl-2,7-naphthyridin-1-yl)-3-methylazetidin-3-ol |
| | Amine: 3-methylazetidin-3-ol |
| | 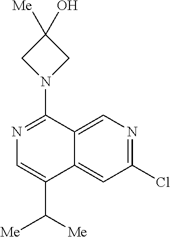 [SMILES: CC(C)c1cnc(N2CC(C)(O)C2)c2cnc(Cl)cc12] |
| | Yellow solid. LCMS m/z = 292 [M + H]⁺ |
| 184 | methyl 2-(1-(6-chloro-4-isopropyl-2,7-naphthyridin-1-yl)azetidin-3-yl)acetate |
| | 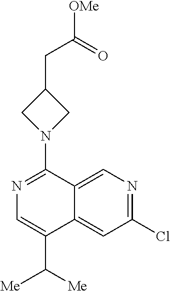 [SMILES: COC(=O)CC1CN(c2ncc(C(C)C)c3cc(Cl)ncc23)C1] |
| | Amine: methyl 2-(azetidin-3-yl)acetate trifluoroacetate |
| | brown solid, 432 mg, 78.4% yield. LCMS m/z = 334 [M + H]⁺ |
| 185 | 6-chloro-1-(3-(difluoromethyl)azetidin-1-yl)-4-isopropyl-2,7-naphthyridine |
| | 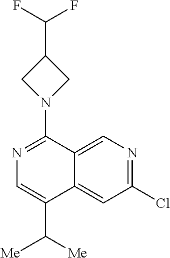 [SMILES: CC(C)c1cnc(N2CC(C(F)F)C2)c2cnc(Cl)cc12] |
| | Amine: 3-(difluoromethyl)azetidine |
| | yellow solid, 230 mg, 87.8%. LCMS m/z = 312 [M + H]⁺ |
| 186 | 6-chloro-1-(1,1-difluoro-5-azaspiro[2.3]hexan-5-yl)-4-isopropyl-2,7- |
| | naphthyridine |
| | 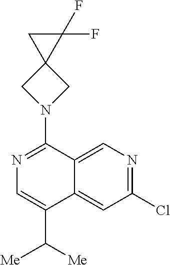 [SMILES: CC(C)c1cnc(N2CC3(C2)CC3(F)F)c2cnc(Cl)cc12] |
| | Amine: 1,1-difluoro-5-azaspiro[2.3]hexane |
| | yellow solid, 210 mg, 78.4% |
| 187 | tert-butyl 1-(6-chloro-4-isopropyl-2,7-naphthyridin-1-yl)-1,6- |
| | diazaspiro[3.4]octane-6-carboxylate |
| | 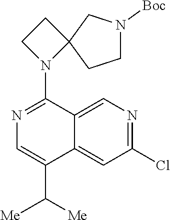 [SMILES: CC(C)c1cnc(N2CCC23CCN(C(=O)OC(C)(C)C)C3)c2cnc(Cl)cc12] |
| | Amine: tert-butyl 1,6-diazaspiro[3.4]octane-6-carboxylate |
| | yellow solid, 200 mg, 68%. LCMS m/z = 417 [M + H]⁺ |
| 188 | tert-butyl 1-(6-chloro-4-isopropyl-2,7-naphthyridin-1-yl)-1,7- |
| | diazaspiro[3.5]nonane-7-carboxylate |
| | 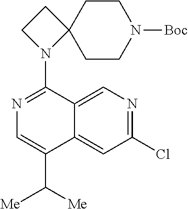 [SMILES: CC(C)c1cnc(N2CCC23CCN(C(=O)OC(C)(C)C)CC3)c2cnc(Cl)cc12] |
| | Amine: tert-butyl 1,7-diazaspiro[3.5]nonane-7-carboxylate |
| | yellow solid, 90 mg, 37.9%. LCMS m/z = 431 [M + H]⁺ |
| 189 | 6-chloro-4-isopropyl-1-(3-(((trifluoromethyl)sulfinyl)methyl)azetidin-1-yl)-2,7- |
| | naphthyridine |
| | 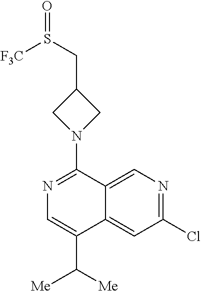 [SMILES: CC(C)c1cnc(N2CC(CS(=O)C(F)(F)F)C2)c2cnc(Cl)cc12] |
| | Amine: 3-(((trifluoromethyl)sulfinyl)methyl)azetidine trifluoroacetate |
| | (Preparation 106) |
| | Light yellow solid, 3 mg, 14.3% yield. LCMS m/z = 392 [M + H]⁺ |
| 190 | tert-butyl (S)-((1-(6-chloro-4-isopropyl-2,7-naphthyridin-1-yl)pyrrolidin-2- |
| | yl)methyl)carbamate |
| | 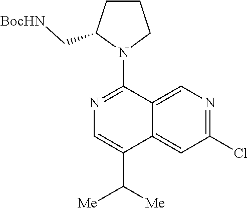 [SMILES: CC(C)c1cnc(N2CCC[C@H]2CNC(=O)OC(C)(C)C)c2cnc(Cl)cc12] |
| | Amine: tert-butyl (S)-(pyrrolidin-2-ylmethyl)carbamate |
| | yellow solid, 400 mg, 66.3%. LCMS m/z = 405 [M + H]⁺ |

### Preparation 191

### (R)-6-chloro-4-isopropyl-1-(2-methylazetidin-1-yl)-2,7-naphthyridine

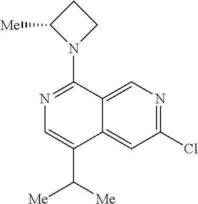 [SMILES: CC(C)c1cnc(N2CC[C@H]2C)c2cnc(Cl)cc12]

A mixture of 1,6-dichloro-4-isopropyl-2,7-naphthyridine (Preparation 148, 300.0 mg, 1.244 mmol), (2R)-2-methylazetidine (88.49 mg, 1.244 mmol) and TEA (377.7 mg, 3.733 mmol) in IPA (2.0 mL), was stirred for 12 h at 100° C. under N₂. The reaction was quenched by the addition of water (3 mL) and the resulting solids were filtered off. The filtrate was concentrated in vacuo and the residue was purified by silica gel column (DCM/MeOH, 15:1) to give the title compound, 250 mg, 72.9% as light yellow oil. LCMS m/z=276 [M+H]⁺

### Preparation 192

### (1-(6-chloro-4-isopropyl-2,7-naphthyridin-1-yl)-3-fluoroazetidin-3-yl)methanol

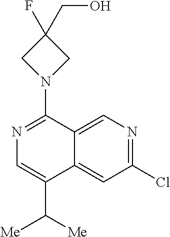 [SMILES: CC(C)c1cnc(N2CC(F)(CO)C2)c2cnc(Cl)cc12]

The title compound was obtained as a yellow solid, 30 mg, 23.3% yield, from 1,6-dichloro-4-isopropyl-2,7-naphthyridine (Preparation 148) and (3-fluoroazetidin-3-yl)methanol following the procedure described in Preparation 191. LCMS m/z=310 [M+H]⁺

### Preparation 193

### 6-chloro-1-(3-fluoro-3-(methoxymethyl)azetidin-1-yl)-4-isopropyl-2,7-naphthyridine

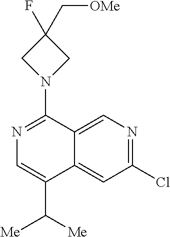 [SMILES: COCC1(F)CN(c2ncc(C(C)C)c3cc(Cl)ncc23)C1]

The title compound was obtained as a yellow solid, 80 mg, 59.7% yield, from 1,6-dichloro-4-isopropyl-2,7-naphthyridine (Preparation 148) and 3-fluoro-3-(methoxymethyl)azetidine following the procedure described in Preparation 191. LCMS m/z=324 [M+H]⁺

### Preparation 194

### ((1-(6-chloro-4-isopropyl-2,7-naphthyridin-1-yl)azetidin-3-yl)imino)dimethyl-λ₆-sulfanone

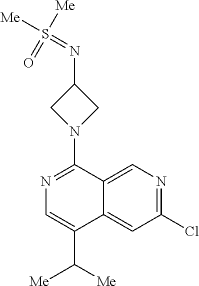 [SMILES: CC(C)c1cnc(N2CC(N=S(C)(C)=O)C2)c2cnc(Cl)cc12]

A mixture of 1,6-dichloro-4-isopropyl-2,7-naphthyridine (Preparation 148, 72.3 mg, 0.30 mmol), (azetidin-3-ylimino)dimethyl-λ₆-sulfanone (Preparation 103, 220 mg, 1.49 mmol) and TEA in DMF (2 mL) was heated to 100° C. for 18 h. The cooled mixture was concentrated in vacuo, and the residue purified by prep-TLC (EtOAc) to give the title compound (20 mg, 19.0%) as a white solid.

### Preparation 195

### (S)-(1-(6-chloro-4-isopropyl-2,7-naphthyridin-1-yl)azetidin-2-yl)methanol

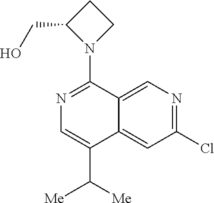 [SMILES: CC(C)c1cnc(N2CC[C@H]2CO)c2cnc(Cl)cc12]

1,6-Dichloro-4-isopropyl-2,7-naphthyridine (Preparation 148, 241.1 mg, 1 mmol), (S)-azetidin-2-ylmethanol (104.5 mg, 1.2 mmol) and DIPEA (323.8 mg, 2.5 mmol) were dissolved in IPA (5 mL) and the reaction stirred at 100° C. for 4 h. The cooled mixture was concentrated in vacuo, and the crude product was purified by column chromatography eluting with 2% MeOH in DCM to provide the title compound, 233 mg, 80%, as a white solid. LCMS m/z=292 [M+H]⁺

### Preparation 196

### 6-chloro-4-isopropyl-1-(3-(methoxymethyl)azetidin-1-yl)-2,7-naphthyridine

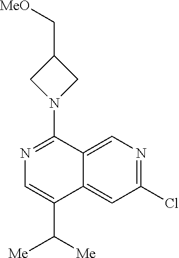 [SMILES: COCC1CN(c2ncc(C(C)C)c3cc(Cl)ncc23)C1]

The title compound was obtained as a white solid, 233 mg, from 1,6-dichloro-4-isopropyl-2,7-naphthyridine (Preparation 148) and 3-(methoxymethyl)azetidine, following a similar procedure to that described in Preparation 195. LCMS m/z=306 [M+H]⁺

### Preparation 197

### (2R,3S)-1-(6-chloro-4-isopropyl-2,7-naphthyridin-1-yl)-2,3-dimethylazetidin-3-ol

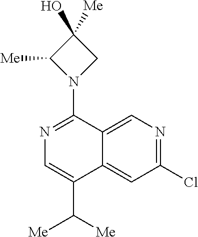 [SMILES: CC(C)c1cnc(N2C[C@](C)(O)[C@H]2C)c2cnc(Cl)cc12]

The title compound was obtained as a light yellow solid, 300 mg, from 1,6-dichloro-4-isopropyl-2,7-naphthyridine (Preparation 148) and (2R,3S)-2,3-dimethylazetidin-3-ol (Preparation 84), following a similar procedure to that described in Preparation 195.

### Preparation 198

### methyl (S)-1-(6-chloro-4-isopropyl-2,7-naphthyridin-1-yl)azetidine-2-carboxylate

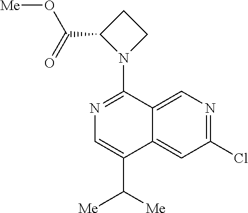 [SMILES: COC(=O)[C@@H]1CCN1c1ncc(C(C)C)c2cc(Cl)ncc12]

The title compound was obtained as a white solid, 240 mg, from 1,6-dichloro-4-isopropyl-2,7-naphthyridine (Preparation 148) and methyl (S)-azetidine-2-carboxylate hydrochloride, following a similar procedure to that described in Preparation 195. LCMS m/z=320 [M+H]⁺

### Preparation 199

### methyl 1-(6-chloro-4-isopropyl-2,7-naphthyridin-1-yl)azetidine-3-carboxylate

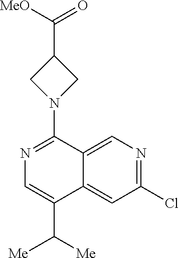 [SMILES: COC(=O)C1CN(c2ncc(C(C)C)c3cc(Cl)ncc23)C1]

The title compound was obtained, 320 mg, from 1,6-dichloro-4-isopropyl-2,7-naphthyridine (Preparation 148) and methyl azetidine-3-carboxylate hydrochloride, following a similar procedure to that described in Preparation 195. LCMS m/z=320 [M+H]⁺

### Preparation 200

### 6-chloro-4-isopropyl-1-((2R,3S)-2-methyl-3-(1H-1,2,3-triazol-1-yl)azetidin-1-yl)-2,7-naphthyridine

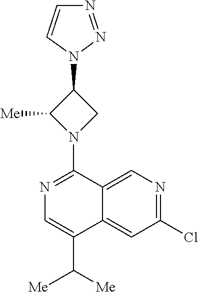 [SMILES: CC(C)c1cnc(N2C[C@H](n3ccnn3)[C@H]2C)c2cnc(Cl)cc12]

To a stirred solution of 1,6-dichloro-4-isopropyl-2,7-naphthyridine (Preparation 148, 60 mg, 0.249 mmol), 1-((2R,3S)-2-methylazetidin-3-yl)-1H-1,2,3-triazole (Preparation 76, 69.0 mg, 0.274 mmol) in IPA (0.96 mL) was added DIPEA (0.109 mL, 0.622 mmol) and the reaction stirred at 90° C. for 3 h. The cooled mixture was concentrated in vacuo, the resulting residue suspended in H₂O and extracted with 5% MeOH in DCM (3×), the organic layers combined, dried over anhydrous Na₂SO₄, filtered and solvent removed under reduced pressure. The crude product was purified by Combiflash® Isco, (0-15% MeOH in DCM) to afford the title compound as a white solid, 64.2 mg, 75% yield. LCMS m/z=343 [M+H]⁺

### Preparations 201 to 212

The compounds in the following table were prepared from 1,6-dichloro-4-isopropyl-2,7-naphthyridine (Preparation 148) and the appropriate amine, following a similar procedure to that described in Preparation 200.

| Prep no | Name, Structure, Amine, data |
|---|---|
| 201 | 1-(6-chloro-4-isopropyl-2,7-naphthyridin-1-yl)-N,N,3-trimethylazetidine-3- |
| | carboxamide |
| | 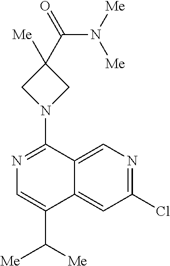 [SMILES: CC(C)c1cnc(N2CC(C)(C(=O)N(C)C)C2)c2cnc(Cl)cc12] |
| | Amine: N,N,3-trimethylazetidine-3-carboxamide (Preparation 98) |
| | 75 mg, 87% yield, LCMS m/z = 347 [M + H]⁺ |
| 202 | 1-(6-chloro-4-isopropyl-2,7-naphthyridin-1-yl)-3-fluoro-N,N-dimethylazetidine-3- |
| | carboxamide |
| | 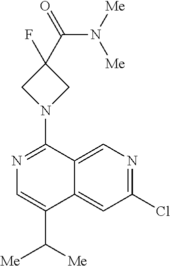 [SMILES: CC(C)c1cnc(N2CC(F)(C(=O)N(C)C)C2)c2cnc(Cl)cc12] |
| | Amine: 3-fluoro-N,N-dimethylazetidine-3-carboxamide (Preparation 100), |
| | colorless oil that solidified on standing, 61 mg, 69.9% yield. LCMS m/z = 341 |
| | [M + H]⁺ |
| 203 | 6-chloro-4-isopropyl-1-(3-(oxetan-3-yl)azetidin-1-yl)-2,7-naphthyridine |
| | 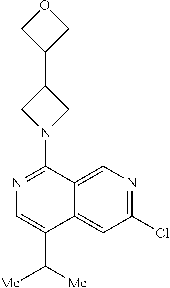 [SMILES: CC(C)c1cnc(N2CC(C3COC3)C2)c2cnc(Cl)cc12] |
| | Amine: 3-(oxetan-3-yl)azetidine |
| | light brown solid, 96.7 mg, 41.1% yield. LCMS m/z = 318 [M + H]⁺ |
| 204 | (1-(6-chloro-4-isopropyl-2,7-naphthyridin-1-yl)azetidin-3-yl)methanamine |
| | 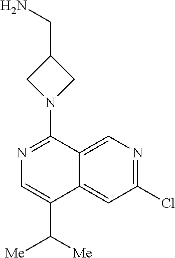 [SMILES: CC(C)c1cnc(N2CC(CN)C2)c2cnc(Cl)cc12] |
| | Amine: 3-aminomethylazetidine |
| 205 | 2-(1-(6-chloro-4-isopropyl-2,7-naphthyridin-1-yl)-3-hydroxyazetidin-3- |
| | yl)acetonitrile |
| | 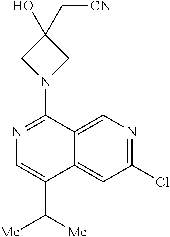 [SMILES: CC(C)c1cnc(N2CC(O)(CC#N)C2)c2cnc(Cl)cc12] |
| | Amine: 2-(3-hydroxyazetidin-3-yl)acetonitrile hydrochloride |
| 206 | 1-(6-chloro-4-isopropyl-2,7-naphthyridin-1-yl)-3-(hydroxymethyl)azetidine-3- |
| | carbonitrile |
| | 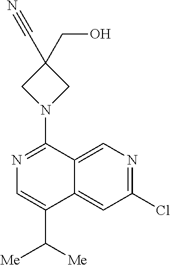 [SMILES: CC(C)c1cnc(N2CC(C#N)(CO)C2)c2cnc(Cl)cc12] |
| | Amine: 3-(hydroxymethyl)azetidine-3-carbonitrile |
| 207 | 6-chloro-4-isopropyl-1-(3-(methylsulfonyl)azetidin-1-yl)-2,7-naphthyridine |
| | 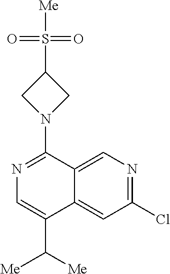 [SMILES: CC(C)c1cnc(N2CC(S(C)(=O)=O)C2)c2cnc(Cl)cc12] |
| | Amine: 3-(methylsulfonyl)azetidine hydrochloride |
| 208 | (S)-6-chloro-4-isopropyl-1-(3-(methylsulfonyl)pyrrolidin-1-yl)-2,7-naphthyridine |
| | 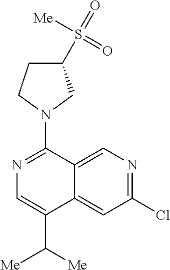 [SMILES: CC(C)c1cnc(N2CC[C@H](S(C)(=O)=O)C2)c2cnc(Cl)cc12] |
| | Amine: (S)-3-(methylsulfonyl)pyrrolidine |
| 209 | (R)-6-chloro-4-isopropyl-1-(3-(methylsulfonyl)pyrrolidin-1-yl)-2,7-naphthyridine |
| | 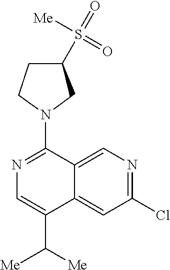 [SMILES: CC(C)c1cnc(N2CC[C@@H](S(C)(=O)=O)C2)c2cnc(Cl)cc12] |
| | Amine: (R)-3-(methylsulfonyl)pyrrolidine |
| 210 | 6-chloro-4-isopropyl-1-(3-((methylsulfonyl)methyl)pyrrolidin-1-yl)-2,7- |
| | naphthyridine |
| | 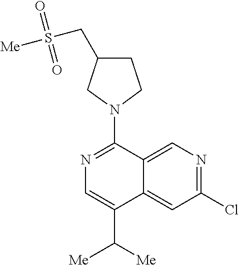 [SMILES: CC(C)c1cnc(N2CCC(CS(C)(=O)=O)C2)c2cnc(Cl)cc12] |
| | Amine: 3-((methylsulfonyl)methyl)pyrrolidine |
| 211 | (2R,3S)-1-(6-chloro-4-isopropyl-2,7-naphthyridin-1-yl)-2-methylpyrrolidin-3-ol |
| | 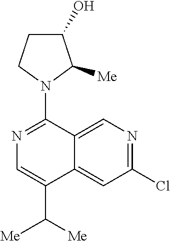 [SMILES: CC(C)c1cnc(N2CC[C@H](O)[C@H]2C)c2cnc(Cl)cc12] |
| | Amine: (2R,3S)-2-methylpyrrolidin-3-ol hydrochloride |
| 212 | 6-chloro-1-(1,1-difluoro-5-azaspiro[2.4]heptan-5-yl)-4-isopropyl-2,7-naphthyridine |
| | 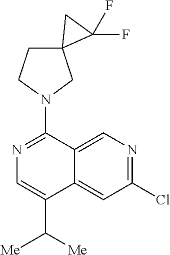 [SMILES: CC(C)c1cnc(N2CCC3(C2)CC3(F)F)c2cnc(Cl)cc12] |
| | Amine: 1,1-difluoro-5-azaspiro[2.4]heptane |

### Preparation 213

### (2R,3S)-1-(6-chloro-4-isopropyl-2,7-naphthyridin-1-yl)-2-methylazetidin-3-ol

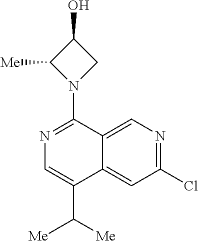 [SMILES: CC(C)c1cnc(N2C[C@H](O)[C@H]2C)c2cnc(Cl)cc12]

DIPEA (0.362 mL, 2.074 mmol) was added to a stirred solution of 1,6-dichloro-4-isopropyl-2,7-naphthyridine (Preparation 148, 200 mg, 0.829 mmol) and (2R,3S)-2-methylazetidin-3-ol ((1S)-7,7-dimethyl-2-oxobicyclo[2.2.1]heptan-1-yl)methanesulfonate (Preparation 72, 291 mg, 0.912 mmol) in IPA (3.19 mL) and the reaction stirred at 90° C. overnight. The cooled mixture was concentrated in vacuo, the residue dissolved in DCM and washed with water. The organic layer was dried over Na₂SO₄, filtered and concentrated in vacuo. The residue was purified by ISCO Combiflash® (0-10% MeOH in DCM) to give the title compound as a yellow solid, 223 mg, 92% yield.

¹H NMR (400 MHz, DMSO-d₆) δ 9.14 (s, 1H), 8.18 (s, 1H), 7.92 (s, 1H), 4.92 (ddd, 1H), 4.47-4.38 (m, 1H), 4.20 (tt, 1H), 3.85 (dd, 1H), 3.42 (p, 1H), 1.45 (d, 3H), 1.30 (dd, 6H)

### Preparations 213A, 214, 215 and 216

### (2R,3S)-1-(6-chloro-4-isopropyl-2,7-naphthyridin-1-yl)-2-methylazetidin-3-ol, (2S,3S)-1-(6-chloro-4-(propan-2-yl)-2,7-naphthyridin-1-yl)-2-methylazetidin-3-ol, (2S,3R)-1-(6-chloro-4-(propan-2-yl)-2,7-naphthyridin-1-yl)-2-methylazetidin-3-ol and (2R,3R)-1-(6-chloro-4-(propan-2-yl)-2,7-naphthyridin-1-yl)-2-methylazetidin-3-ol

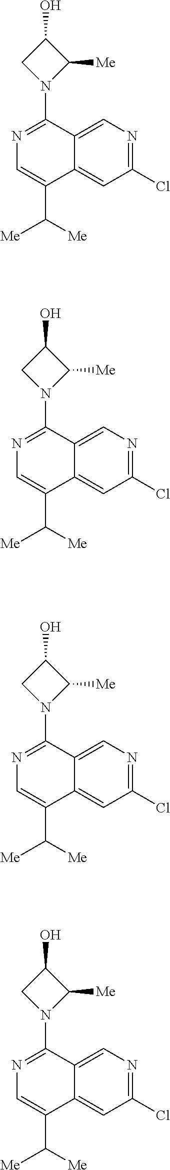 [SMILES: CC(C)c1cnc(N2C[C@H](O)[C@H]2C)c2cnc(Cl)cc12 ; CC(C)c1cnc(N2C[C@@H](O)[C@@H]2C)c2cnc(Cl)cc12 ; CC(C)c1cnc(N2C[C@H](O)[C@@H]2C)c2cnc(Cl)cc12 ; CC(C)c1cnc(N2C[C@@H](O)[C@H]2C)c2cnc(Cl)cc12]

1-(6-Chloro-4-isopropyl-2,7-naphthyridin-1-yl)-2-methylazetidin-3-ol (Preparation 178, 600 mg, 2.05 mmol) was purified by preparative SFC using a CHIRALPAK AD-3 3×100 mm, 3 μm column, mobile phase 10-50% EtOH (0.1% DEA) over 4.0 min, at 2 mL/min, to give:

Peak 1: (2R,3S)-1-(6-chloro-4-isopropyl-2,7-naphthyridin-1-yl)-2-methylazetidin-3-ol or (2S,3S)-1-(6-chloro-4-(propan-2-yl)-2,7-naphthyridin-1-yl)-2-methylazetidin-3-ol (90 mg) as a white solid,

Peak 2: (2S,3S)-1-(6-chloro-4-isopropyl-2,7-naphthyridin-1-yl)-2-methylazetidin-3-ol or (2R,3S)-1-(6-chloro-4-(propan-2-yl)-2,7-naphthyridin-1-yl)-2-methylazetidin-3-ol (90 mg) as a white solid,

Peak 3: (2S,3R)-1-(6-chloro-4-isopropyl-2,7-naphthyridin-1-yl)-2-methylazetidin-3-ol or (2R,3R)-1-(6-chloro-4-isopropyl-2,7-naphthyridin-1-yl)-2-methylazetidin-3-ol (90 mg) as a white solid,

Peak 4: (2R,3R)-1-(6-chloro-4-isopropyl-2,7-naphthyridin-1-yl)-2-methylazetidin-3-ol or (2S,3R)-1-(6-chloro-4-isopropyl-2,7-naphthyridin-1-yl)-2-methylazetidin-3-ol (90 mg) as a white solid.

### Preparation 217

### 6-(6-chloro-4-isopropyl-2,7-naphthyridin-1-yl)-1-thia-6-azaspiro[3.3]heptane 1,1-dioxide

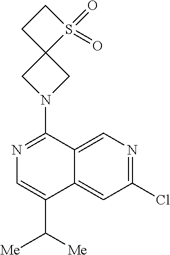 [SMILES: CC(C)c1cnc(N2CC3(CCS3(=O)=O)C2)c2cnc(Cl)cc12]

A mixture of 1,6-dichloro-4-isopropyl-2,7-naphthyridine (Preparation 148, 78 mg, 0.323 mmol), 1-thia-6-azaspiro[3.3]heptane 1,1-dioxide (65 mg, 0.356 mmol) and DIPEA (105 mg, 0.809 mmol) in IPA (1 mL) was heated to 60° C. overnight. The cooled reaction mixture was filtered, washed with IPA, then water and dried in vacuo to afford the title compound, as a white solid, 75 mg, 66%. LCMS m/z=352 [M+H]⁺

### Preparation 218

### 2-(6-chloro-4-isopropyl-2,7-naphthyridin-1-yl)-7-methyl-5-oxa-2,7-diazaspiro[3.4]octan-6-one

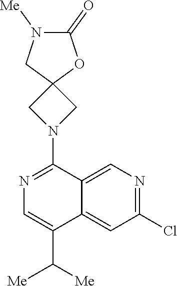 [SMILES: CC(C)c1cnc(N2CC3(CN(C)C(=O)O3)C2)c2cnc(Cl)cc12]

The title compound was obtained as an off-white solid, 47 mg, 65.4% yield, from 7-methyl-5-oxa-2,7-diazaspiro[3.4]octan-6-one hydrochloride, and 1,6-dichloro-4-isopropyl-2,7-naphthyridine (Preparation 148), following a similar procedure to that described in Preparation 217. LCMS m/z=347 [M+H]⁺

### Preparation 219

### 6-chloro-1-(cis-3,4-difluoropyrrolidin-1-yl)-4-isopropyl-2,7-naphthyridine

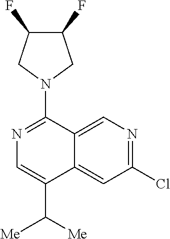 [SMILES: CC(C)c1cnc(N2C[C@@H](F)[C@@H](F)C2)c2cnc(Cl)cc12]

A mixture of 1,6-dichloro-4-isopropyl-2,7-naphthyridine (Preparation 148, 100 mg, 0.414 mmol), (3r,4s)-3,4-difluoropyrrolidine (44.3 mg, 0.414 mmol) and DIPEA (159 mg, 1.24 mmol) in DMSO (3 mL) was stirred at 100° C. for 2 h. The cooled reaction mixture was diluted with water (100 mL), extracted with EtOAc and the combined organic extracts washed with brine (100 mL). The organic layer was dried over Na₂SO₄, filtered, evaporated and purified by silica gel column chromatography (DCM:MeOH=20:1) to afford the title compound, 110 mg, 85.1% yield as a yellow solid. LCMS m/z=312 [M+H]⁺

### Preparation 220

### 6-chloro-1-(trans-3,4-difluoropyrrolidin-1-yl)-4-isopropyl-2,7-naphthyridine

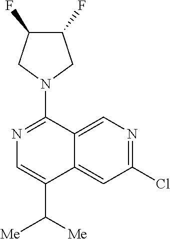 [SMILES: CC(C)c1cnc(N2C[C@@H](F)[C@H](F)C2)c2cnc(Cl)cc12]

The title compound was obtained as a yellow solid, 230 mg, 88.9% yield from 1,6-dichloro-4-isopropyl-2,7-naphthyridine (Preparation 148) and (3r,4r)-3,4-difluoropyrrolidine, following the procedure described in Preparation 219. LCMS m/z=312 [M+H]⁺

### Preparations 221 and 222

### 6-chloro-1-((3R,4R)-3,4-difluoropyrrolidin-1-yl)-4-isopropyl-2,7-naphthyridine and 6-chloro-1-((3S,4S)-3,4-difluoropyrrolidin-1-yl)-4-isopropyl-2,7-naphthyridine

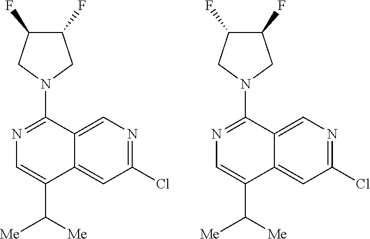 [SMILES: CC(C)c1cnc(N2C[C@@H](F)[C@H](F)C2)c2cnc(Cl)cc12 ; CC(C)c1cnc(N2C[C@H](F)[C@@H](F)C2)c2cnc(Cl)cc12]

6-Chloro-1-(trans-3,4-difluoropyrrolidin-1-yl)-4-isopropyl-2,7-naphthyridine (Preparation 220, 230 mg, 0.737 mmol) was purified by Chiral HPLC using a CHIRALPAK IG-3 4.6×50 mm 3 μm column, mobile phase: Hex(0.1% DEA):EtOH=50:50 at 1 mL/min to give

Peak 1: 6-chloro-1-((3R,4R)-3,4-difluoropyrrolidin-1-yl)-4-isopropyl-2,7-naphthyridine or 6-chloro-1-((3S,4S)-3,4-difluoropyrrolidin-1-yl)-4-isopropyl-2,7-naphthyridine (60 mg) as a white solid and

Peak 2: 6-chloro-1-((3S,4S)-3,4-difluoropyrrolidin-1-yl)-4-isopropyl-2,7-naphthyridine and 6-chloro-1-((3R,4R)-3,4-difluoropyrrolidin-1-yl)-4-isopropyl-2,7-naphthyridine (60 mg) as a white solid.

### Preparation 223

### 1-(6-chloro-4-isopropyl-2,7-naphthyridin-1-yl)-3-fluoroazetidine-3-carboxylic acid

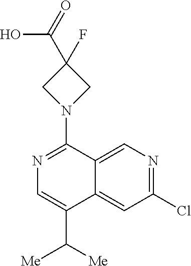 [SMILES: CC(C)c1cnc(N2CC(F)(C(=O)O)C2)c2cnc(Cl)cc12]

A mixture of 1,6-dichloro-4-isopropyl-2,7-naphthyridine (Preparation 148, 150 mg, 0.622 mmol), 3-fluoroazetidine-3-carboxylic acid (81 mg 0.684 mmol) and DIPEA (201 mg, 1.555 mmol) in IPA (2 mL) was heated under reflux overnight. The cooled mixture was diluted with water and the pH adjusted to 3 using 1M HCl. The solution was extracted with 10% MeOH/DCM (2×), the combined organics dried over Na₂SO₄, filtered and evaporated under reduced pressure to give the title compound, 161 mg, 80%, as a yellow foam. LCMS m/z=324 [M+H]⁺

### Preparation 224

### Trans-rac-1-(6-chloro-4-isopropyl-2,7-naphthyridin-1-yl)-2-methylazetidine-3-carboxylic acid

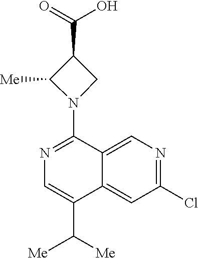 [SMILES: CC(C)c1cnc(N2C[C@H](C(=O)O)[C@H]2C)c2cnc(Cl)cc12]

The title compound was obtained from trans-rac 2-methylazetidine-3-carboxylic acid and 1,6-dichloro-4-isopropyl-2,7-naphthyridine (Preparation 148) following the procedure described in Preparation 223. LCMS m/z=320 [M+H]⁺

### Preparation 225

### 1-(6-chloro-4-isopropyl-2,7-naphthyridin-1-yl)-3-fluoro-N-methylazetidine-3-carboxamide

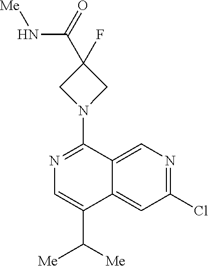 [SMILES: CNC(=O)C1(F)CN(c2ncc(C(C)C)c3cc(Cl)ncc23)C1]

A solution of 1-(6-chloro-4-isopropyl-2,7-naphthyridin-1-yl)-3-fluoroazetidine-3-carboxylic acid (Preparation 223, 33 mg, 0.102 mmol), HATU (50 mg, 0.133 mmol), methanamine (76 μL, 0.153 mmol) and DIPEA (53 μL, 0.306 mmol) in THF (1 mL) was stirred at rt overnight. The mixture was diluted with EtOAc, washed with water and brine, then dried over Na₂SO₄. The filtrate was evaporated under reduced pressure to give the title compound as an off-white solid, 28 mg, 82%. LCMS m/z=337 [M+H]⁺

### Preparation 226

### Trans-rac-1-(6-chloro-4-isopropyl-2,7-naphthyridin-1-yl)-N,N,2-trimethylazetidine-3-carboxamide

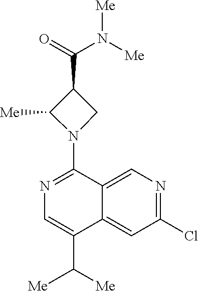 [SMILES: CC(C)c1cnc(N2C[C@H](C(=O)N(C)C)[C@H]2C)c2cnc(Cl)cc12]

The title compound was obtained from methylamine and trans-rac-1-(6-chloro-4-isopropyl-2,7-naphthyridin-1-yl)-2-methylazetidine-3-carboxylic acid (Preparation 224) following the procedure described in Preparation 225.

### Preparation 227

### N-((1-(6-chloro-4-isopropyl-2,7-naphthyridin-1-yl)azetidin-3-yl)methyl)methanesulfonamide

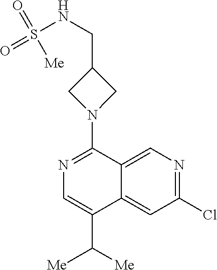 [SMILES: CC(C)c1cnc(N2CC(CNS(C)(=O)=O)C2)c2cnc(Cl)cc12]

The title compound was obtained from (1-(6-chloro-4-isopropyl-2,7-naphthyridin-1-yl)azetidin-3-yl)methanamine (Preparation 204) and mesyl chloride following a similar procedure to that described in Preparation 140.

### Preparation 228

### 6-chloro-1-(3-(iodomethyl)azetidin-1-yl)-4-isopropyl-2,7-naphthyridine

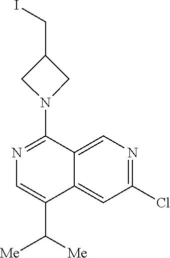 [SMILES: CC(C)c1cnc(N2CC(CI)C2)c2cnc(Cl)cc12]

To a solution of (1-(6-chloro-4-isopropyl-2,7-naphthyridin-1-yl)azetidin-3-yl)methanol (Preparation 162, 190 mg, 0.651 mmol), PPh₃ (255 mg, 0.976 mmol) and imidazole (66.4 mg, 0.976 mmol) in DCM (30 mL) was added iodine (247 mg, 0.976 mmol) at 0° C. and the reaction stirred at rt for 3 h. The reaction was quenched by the addition of sat. aq. Na₂SO₃ solution, the layers separated and the organic phase dried over Na₂SO₄. The organic solution was filtered and concentrated in vacuo and the residue purified by prep-TLC (DCM:MeOH=20:1) to afford the title compound (120 mg, 45.8%) LCMS m/z=402 [M+H]⁺

### Preparation 229

### dimethyl ((1-(6-chloro-4-isopropyl-2,7-naphthyridin-1-yl)azetidin-3-yl)methyl)phosphonate

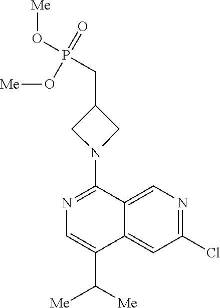 [SMILES: COP(=O)(CC1CN(c2ncc(C(C)C)c3cc(Cl)ncc23)C1)OC]

A solution of 6-chloro-1-(3-(iodomethyl)azetidin-1-yl)-4-isopropyl-2,7-naphthyridine (Preparation 228, 800 mg, 1.99 mmol), dimethyl phosphonate (327 mg, 2.98 mmol) and Cs₂CO₃ (976 mg, 2.98 mmol) in DMF (10 mL) was stirred at 80° C. for 1 h. The reaction mixture was diluted with water and extracted with EtOAc. The combined organic layers were dried over Na₂SO₄, filtered and concentrated in vacuo. The residue was purified by prep TLC (DCM:MeOH=10:1) to afford the title compound, 600 mg, 78.5% as a light yellow oil. LCMS m/z=384 [M+H]⁺

### Preparation 230

### (S)-6-chloro-4-isopropyl-1-(2-(methoxymethyl)azetidin-1-yl)-2,7-naphthyridine

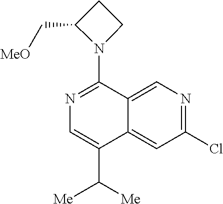 [SMILES: COC[C@@H]1CCN1c1ncc(C(C)C)c2cc(Cl)ncc12]

A mixture of (S)-(1-(6-chloro-4-isopropyl-2,7-naphthyridin-1-yl)azetidin-2-yl)methanol (Preparation 195, 87.5 mg, 0.3 mmol), Mel (85.2 mg, 0.6 mmol) and NaH (30 mg, 0.75 mmol) in THF (5 mL) was stirred at 60° C. for 4 h. The reaction mixture was concentrated in vacuo and the crude product was purified by column chromatography (MeOH-DCM, 1:100) to give the title compound, 61 mg, 66.4% as a white solid. LCMS m/z=306 [M+H]⁺

### Preparation 231

### tert-butyl (S)-((1-(6-chloro-4-isopropyl-2,7-naphthyridin-1-yl)azetidin-2-yl)methyl)(methyl)carbamate

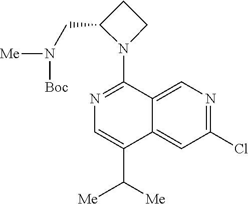 [SMILES: CC(C)c1cnc(N2CC[C@H]2CN(C)C(=O)OC(C)(C)C)c2cnc(Cl)cc12]

A mixture of tert-butyl (S)-((1-(6-chloro-4-isopropyl-2,7-naphthyridin-1-yl)azetidin-2-yl)methyl)carbamate (Preparation 171, 100 mg, 0.256 mmol), NaH (30.4 mg, 1.27 mmol) and Mel (180 mg, 1.27 mmol) in THF (10 mL) was stirred at rt for 3 h. The mixture was quenched with water, and extracted with EtOAc. The organic phase was concentrated in vacuo and the residue purified by prep TLC (PE:EtOAc=4:1) to afford the title compound, 110 mg, crude. LCMS m/z=405 [M+H]⁺

### Preparation 232

### tert-butyl (S)-((1-(6-chloro-4-isopropyl-2,7-naphthyridin-1-yl)pyrrolidin-2-yl)methyl)(methyl)carbamate

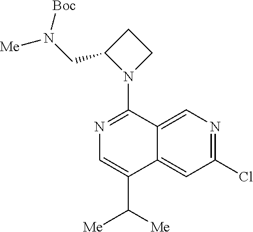 [SMILES: CC(C)c1cnc(N2CC[C@H]2CN(C)C(=O)OC(C)(C)C)c2cnc(Cl)cc12]

The title compound was obtained as a yellow solid, 300 mg, 72.6% yield, from tert-butyl (S)-((1-(6-chloro-4-isopropyl-2,7-naphthyridin-1-yl)pyrrolidin-2-yl)methyl)carbamate (Preparation 190), following a similar procedure to that described in Preparation 231. LCMS m/z=419 [M+H]⁺

### Preparation 233

### 1-((2R,3S)-3-(2-((tert-butyldimethylsilyl)oxy)ethoxy)-2-methylazetidin-1-yl)-6-chloro-4-isopropyl-2,7-naphthyridine

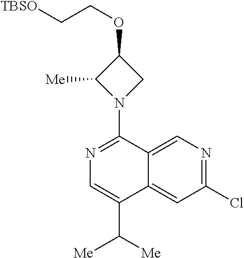 [SMILES: CC(C)c1cnc(N2C[C@H](OCCO[Si](C)(C)C(C)(C)C)[C@H]2C)c2cnc(Cl)cc12]

The title compound was obtained as a white solid, 600 mg, 35.5% yield, from (2R,3S)-1-(6-chloro-4-isopropyl-2,7-naphthyridin-1-yl)-2-methylazetidin-3-ol (Preparation 213) and (2-bromoethoxy)(tert-butyl)dimethylsilane following a similar procedure to that described in Preparation 24. LCMS m/z=450 [M+H]⁺

### Preparation 234

### 2-(((2R,3S)-1-(6-chloro-4-isopropyl-2,7-naphthyridin-1-yl)-2-methylazetidin-3-yl)oxy)ethan-1-ol

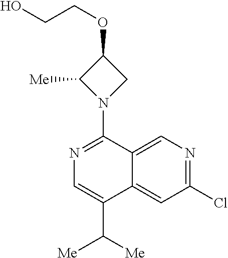 [SMILES: CC(C)c1cnc(N2C[C@H](OCCO)[C@H]2C)c2cnc(Cl)cc12]

A mixture of 1-((2R,3S)-3-(2-((tert-butyldimethylsilyl)oxy)ethoxy)-2-methylazetidin-1-yl)-6-chloro-4-isopropyl-2,7-naphthyridine (Preparation 233, 600 mg, 1.33 mmol) and tetra-n-butylammonium fluoride (3 mL) in THF (6 mL) was stirred at rt for 2 h. The solution was concentrated in vacuo and purified by prep-TLC with DCM/MeOH (20:1) to give the title compound, 400 mg, 89.5% as an off-white solid. LCMS m/z=336 [M+H]⁺

### Preparation 235

### methyl ((1-(6-chloro-4-isopropyl-2,7-naphthyridin-1-yl)azetidin-3-yl)methyl)carbamate

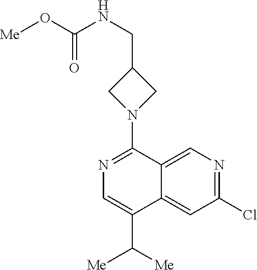 [SMILES: COC(=O)NCC1CN(c2ncc(C(C)C)c3cc(Cl)ncc23)C1]

A mixture of (1-(6-chloro-4-isopropyl-2,7-naphthyridin-1-yl)azetidin-3-yl)methanamine (Preparation 204, 58 mg, 0.2 mmol), DIPEA (0.105 mL, 0.6 mmol) and methyl chloroformate (19 mg, 0.2 mmol) in dioxane (0.667 mL) was stirred at rt. The mixture was concentrated in vacuo, and the residue purified by Isco chromatography to provide the title compound.

### Preparation 236

### 6-chloro-4-isopropyl-1-(1,6-diazaspiro[3.3]heptan-1-yl)-2,7-naphthyridine trifluoroacetate

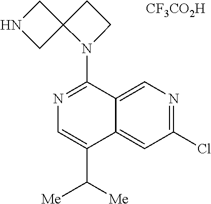 [SMILES: CC(C)c1cnc(N2CCC23CNC3)c2cnc(Cl)cc12 ; "CF3CO2H"]

To a solution of tert-butyl 1-(6-chloro-4-isopropyl-2,7-naphthyridin-1-yl)-1,6-diazaspiro[3.3]heptane-6-carboxylate (Preparation 158, 100 mg, 0.2481 mmol) in DCM (3 mL) was added TFA (1 mL) and the reaction stirred for 1 h at rt. The resulting solution was evaporated under reduced pressure to give the title compound, as a yellow oil, 70 mg. LCMS m/z=303 [M+H]⁺

### Preparation 237

### 6-chloro-4-isopropyl-1-(6-methyl-1,6-diazaspiro[3.3]heptan-1-yl)-2,7-naphthyridine

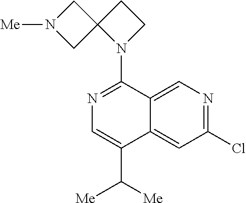 [SMILES: CC(C)c1cnc(N2CCC23CN(C)C3)c2cnc(Cl)cc12]

To a solution of 6-chloro-4-isopropyl-1-(1,6-diazaspiro[3.3]heptan-1-yl)-2,7-naphthyridine trifluoroacetate (Preparation 236, 80 mg, 0.264 mmol) in MeOH was added HCHO (39.5 mg, 1.32 mmol), AcOH (15.8 mg, 0.264 mmol) and NaBH₃CN (49.9 mg, 0.79 mmol) and the reaction stirred for 2 h at rt. The resulting solution was diluted with water (50 mL), extracted with EtOAc (2×50 mL) the organic layers combined and washed with brine (50 mL). The mixture was dried over anhydrous Na₂SO₄ and concentrated in vacuo. The crude product was purified by TLC to give the title compound, 60 mg, 71.7%, as yellow solid. LCMS m/z=317 [M+H]⁺

### Preparation 238

### 6-chloro-4-isopropyl-1-(6-(2-methoxyethyl)-1,6-diazaspiro[3.3]heptan-1-yl)-2,7-naphthyridine

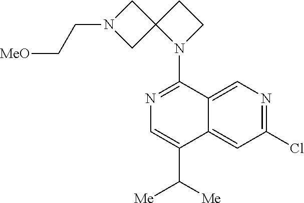 [SMILES: COCCN1CC2(CCN2c2ncc(C(C)C)c3cc(Cl)ncc23)C1]

To a solution of 6-chloro-4-isopropyl-1-(1,6-diazaspiro[3.3]heptan-1-yl)-2,7-naphthyridine trifluoroacetate (Preparation 236, 50 mg, 0.165 mmol) in IPA was added 1-bromo-2-methoxyethane (45.8 mg, 0.33 mmol) and TEA (83.3 mg, 0.82 mmol) and the reaction stirred for 16 h at 50° C. The cooled solution was diluted with water (100 mL), extracted with EtOAc (2×100 mL) and the organic layers combined. The organic solution was washed with brine (50 mL), dried over anhydrous Na₂SO₄ and concentrated in vacuo. The product was purified by column chromatography on silica gel eluting with PE:EtOAc (2: 1) to afford the title compound, 50 mg, 84% as yellow solid. LCMS m/z=361 [M+H]⁺

### Preparation 239

### methyl 1-(6-chloro-4-isopropyl-2,7-naphthyridin-1-yl)-1,6-diazaspiro[3.3]heptane-6-carboxylate

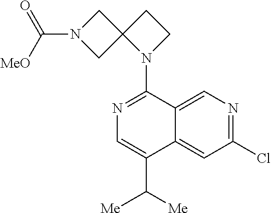 [SMILES: COC(=O)N1CC2(CCN2c2ncc(C(C)C)c3cc(Cl)ncc23)C1]

The title compound was obtained from 6-chloro-4-isopropyl-1-(1,6-diazaspiro[3.3]heptan-1-yl)-2,7-naphthyridine trifluoroacetate (Preparation 236, 39 mg, 0.13 mmol) and methyl chloroformate, following a similar procedure to that described in Preparation 235.

### Preparation 240

### (S)-1-(6-chloro-4-isopropyl-2,7-naphthyridin-1-yl)azetidine-2-carboxylic acid

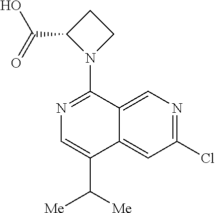 [SMILES: CC(C)c1cnc(N2CC[C@H]2C(=O)O)c2cnc(Cl)cc12]

Methyl (S)-1-(6-chloro-4-isopropyl-2,7-naphthyridin-1-yl)azetidine-2-carboxylate (Preparation 198, 240 mg, 0.750 mmol) was dissolved in 2 M LiOH THF-H₂O (1:1, 5 mL) and the reaction stirred at 25° C. for 4 h. The mixture was acidified using 2N HCl and extracted with EtOAc. The organic phase was washed with brine, dried and concentrated in vacuo. The crude product was purified by silica gel column eluting with MeOH-DCM (2:1) to provide the title compound, 205 mg, 89.3%, as a white solid. LCMS m/z=306 [M+H]⁺

### Preparation 241

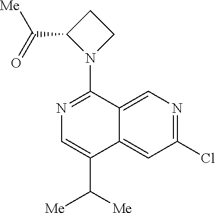 [SMILES: CC(=O)[C@@H]1CCN1c1ncc(C(C)C)c2cc(Cl)ncc12]

### (S)-1-(1-(6-chloro-4-isopropyl-2,7-naphthyridin-1-yl)azetidin-2-yl)ethan-1-one

Part A: (S)-1-(6-Chloro-4-isopropyl-2,7-naphthyridin-1-yl)azetidine-2-carboxylic acid (Preparation 240, 170 mg, 0.555 mmol), CDI (134 mg, 0.832 mmol) and TEA (167 mg, 1.66 mmol) were dissolved in DCM (5 mL) and the reaction stirred at 25° C. for 1 h. Methoxy(methyl)amine hydrochloride (81.1 mg, 0.832 mmol) was added and the reaction stirred for a further 4 h. The reaction was diluted with water, extracted with DCM, the organic phase was dried over Na₂SO₄, and concentrated in vacuo. The crude product was purified by silica gel column eluting with MeOH-DCM (1:50) to give (S)-1-(6-chloro-4-isopropyl-2,7-naphthyridin-1-yl)-N-methoxy-N-methylazetidine-2-carboxamide, 150 mg as a white solid.

Part B: Bromo(methyl)magnesium was added dropwise to a solution of (S)-1-(6-chloro-4-isopropyl-2,7-naphthyridin-1-yl)-N-methoxy-N-methylazetidine-2-carboxamide (150 mg, 0.43 mmol) in THF (5 mL) and the reaction stirred at 25° C. for 1 h. The reaction was quenched with water, extracted with EtOAc, the combined organic extracts were dried over Na₂SO₄, and concentrated in vacuo. The crude product was purified by silica gel column eluting with MeOH-DCM (1:50) to give the title compound, 115 mg as a white solid. LCMS m/z=304 [M+H]⁺

### Preparation 242

### 1-((S)-1-(6-chloro-4-isopropyl-2,7-naphthyridin-1-yl)azetidin-2-yl)ethan-1-ol

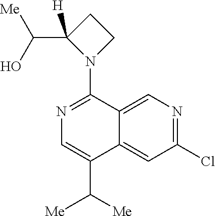 [SMILES: CC(C)c1cnc(N2CC[C@H]2C(C)O)c2cnc(Cl)cc12]

A solution of (S)-1-(1-(6-chloro-4-isopropyl-2,7-naphthyridin-1-yl)azetidin-2-yl)ethan-1-one (Preparation 241, 100 mg, 0.328 mmol) in THF (5 mL) was cooled to 0° C. LiAlH₄ (18.6 mg, 0.492 mmol) was added and the reaction stirred at 0° C. for 1 h. The reaction was quenched with water, extracted with EtOAc, the organic phase was dried over Na₂SO₄, and the solution concentrated in vacuo. The crude product was purified by silica gel column eluting with MeOH-DCM (1:50) to give the title compound, 45 mg, 44.8%, as a white solid. LCMS m/z=306 [M+H]⁺

### Preparation 243

### 1-(6-chloro-4-isopropyl-2,7-naphthyridin-1-yl)azetidine-3-carboxylic acid

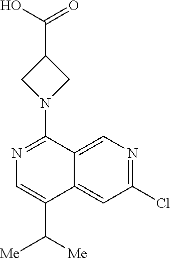 [SMILES: CC(C)c1cnc(N2CC(C(=O)O)C2)c2cnc(Cl)cc12]

The title compound was obtained, 180 mg, 7% yield, from methyl 1-(6-chloro-4-isopropyl-2,7-naphthyridin-1-yl)azetidine-3-carboxylate (Preparation 199) following the procedure described in Preparation 240. LCMS: m/z=306 [M+H]⁺

### Preparation 244

### 2-(1-(6-chloro-4-isopropyl-2,7-naphthyridin-1-yl)azetidin-3-yl)acetic acid

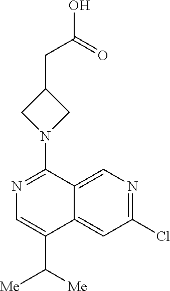 [SMILES: CC(C)c1cnc(N2CC(CC(=O)O)C2)c2cnc(Cl)cc12]

The title compound was obtained, 230 mg, as a brown solid from methyl 2-(1-(6-chloro-4-isopropyl-2,7-naphthyridin-1-yl)azetidin-3-yl)acetate (Preparation 184), following a similar procedure to that described in Preparation 240. LCMS m/z=320 [M+H]⁺

### Preparation 245

### 1-(6-chloro-4-isopropyl-2,7-naphthyridin-1-yl)-N-methylazetidine-3-carboxamide

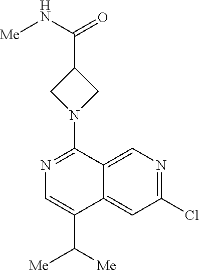 [SMILES: CNC(=O)C1CN(c2ncc(C(C)C)c3cc(Cl)ncc23)C1]

To a mixture of 1-(6-chloro-4-isopropyl-2,7-naphthyridin-1-yl)azetidine-3-carboxylic acid (Preparation 243, 150 mg, 0.490 mmol), HOBT (99.2 mg, 0.735 mmol) and EDC·HCl (93.9 mg, 0.490 mmol) in DCM was stirred at 0° C. for 1 h. Methylamine hydrochloride (49.2 mg, 0.735 mmol) was added and the reaction stirred at rt for 12 h. The mixture was washed with water, extracted with EtOAc and the combined organic extracts evaporated under reduced pressure. The crude was purified by silica gel column (5% MeOH in DCM) to provide the title compound, 100 mg, 63.9%. LCMS m/z=319 [M+H]⁺

### Preparation 246

### 2-(1-(6-chloro-4-isopropyl-2,7-naphthyridin-1-yl)azetidin-3-yl)-N,N-dimethylacetamide

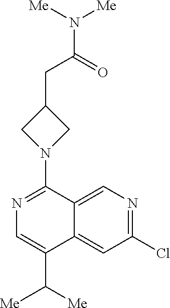 [SMILES: CC(C)c1cnc(N2CC(CC(=O)N(C)C)C2)c2cnc(Cl)cc12]

Dimethylamine (28.1 mg, 0.625 mmol), HATU (356 mg, 0.937 mmol) and DIPEA (241 mg, 1.87 mmol) were added to a solution of 2-(1-(6-chloro-4-isopropyl-2,7-naphthyridin-1-yl)azetidin-3-yl)acetic acid (Preparation 244, 200 mg, 0.625 mmol) in DCM (10 mL) and the reaction stirred for 3 h at rt. The reaction was quenched with water and extracted with EtOAc. The organic layers were combined, concentrated in vacuo and the residue purified by silica gel column eluting with PE/EtOAc (1/1) to give the title compound as a yellow solid (80.0 mg, 36.9%). LCMS m/z=347 [M+H]⁺

### Preparation 247

### 2-(1-(6-chloro-4-isopropyl-2,7-naphthyridin-1-yl)azetidin-3-yl)-N-methylacetamide

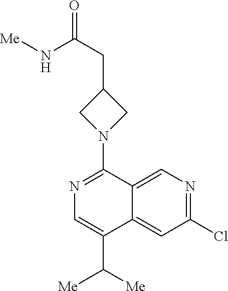 [SMILES: CNC(=O)CC1CN(c2ncc(C(C)C)c3cc(Cl)ncc23)C1]

The title compound was obtained as a brown oil, 78 mg, 62.5% yield, from 2-(1-(6-chloro-4-isopropyl-2,7-naphthyridin-1-yl)azetidin-3-yl)acetic acid (Preparation 244) and methylamine, following the procedure described in Preparation 246. LCMS m/z=333 [M+H]⁺

### Preparation 248

### 6-chloro-1-(3-fluoro-2-methylazetidin-1-yl)-4-isopropyl-2,7-naphthyridine

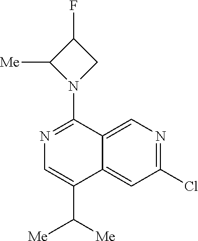 [SMILES: CC(C)c1cnc(N2CC(F)C2C)c2cnc(Cl)cc12]

DAST (688 mg, 4.27 mmol) was added to a solution of 1-(6-chloro-4-isopropyl-2,7-naphthyridin-1-yl)-2-methylazetidin-3-ol (Preparation 178, 500 mg, 1.71 mmol) in DCM (10 mL) and the reaction stirred at 0° C. for 3 h. The reaction was quenched with water, the mixture extracted with EtOAc and the combined organic layers were concentrated in vacuo. The residue was purified by silica gel column eluting with PE/EtOAc (5/1) to give the title compound as a brown solid, 478 mg, 95%. LCMS m/z=294 [M+H]⁺

### Preparation 249

### 6-chloro-1-((2R,3S)-3-(difluoromethoxy)-2-methylazetidin-1-yl)-4-isopropyl-2,7-naphthyridine

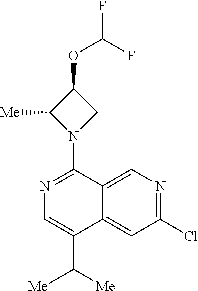 [SMILES: CC(C)c1cnc(N2C[C@H](OC(F)F)[C@H]2C)c2cnc(Cl)cc12]

A solution of (2R,3S)-1-(6-chloro-4-isopropyl-2,7-naphthyridin-1-yl)-2-methylazetidin-3-ol (Preparation 213, 50 mg, 0.171 mmol) and CuI (6.53 mg, 0.034 mmol) in MeCN (0.5 mL) was heated to 50° C. under N₂. 2,2-Difluoro-2-(fluorosulfonyl)acetic acid (0.026 mL, 0.257 mmol) in MeCN (1.0 mL) was added dropwise over 3 mins and the reaction stirred for 3 h. The reaction was evaporated under reduced pressure and the crude purified by Isco Combiflash® (0-10% MeOH in DCM) to afford the title compound, 18.4 mg, 31.4% yield. LCMS m/z=342 [M+H]⁺

### Preparation 250

### 6-chloro-4-isopropyl-1-((2R,3S)-3-(methoxy-d3)-2-methylazetidin-1-yl)-2,7-naphthyridine

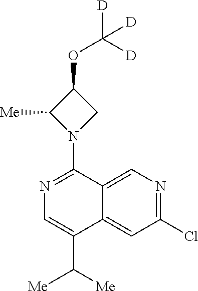 [SMILES: [2H]C([2H])([2H])O[C@H]1CN(c2ncc(C(C)C)c3cc(Cl)ncc23)[C@@H]1C]

A solution of (2R,3S)-1-(6-chloro-4-isopropyl-2,7-naphthyridin-1-yl)-2-methylazetidin-3-ol (Preparation 213, 100 mg, 0.343 mmol) in DMF (1.83 mL) was cooled to 0° C., NaH (16.45 mg, 0.411 mmol) added, the solution stirred for 5 mins, then iodomethane-d3 (0.085 mL, 1.371 mmol) was added. The reaction was stirred at rt for 2 h, then additional NaH and iodomethane-d3 added and the reaction stirred overnight. The reaction was quenched by slow addition of water and the mixture was extracted with EtOAc (3×10 mL). The combined organic layers were washed with brine (15 mL), dried over anhydrous Na₂SO₄, filtered and the solvent removed in vacuo. The crude product was purified by Isco Combiflash® (0-10% MeOH in DCM) to afford the title compound as a yellow solid, 37 mg, 35% yield. LCMS m/z=309 [M+H]⁺

### Preparation 251

### 6-chloro-4-isopropyl-1-((2R,3S)-3-methoxy-2-methylazetidin-1-yl)-2,7-naphthyridine

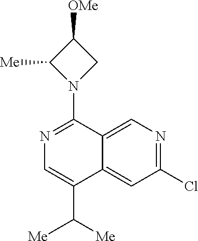 [SMILES: CO[C@H]1CN(c2ncc(C(C)C)c3cc(Cl)ncc23)[C@@H]1C]

The title compound was obtained as a yellow solid, 96 mg, 92% yield, from (2R,3S)-1-(6-chloro-4-isopropyl-2,7-naphthyridin-1-yl)-2-methylazetidin-3-ol (Preparation 213) and iodomethane, following the procedure described in Preparation 250. LCMS m/z=306 [M+H]⁺

### Preparation 252

### 6-chloro-4-(prop-1-en-2-yl)-2,7-naphthyridin-1(2H)-one

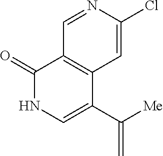

A mixture of 6-chloro-4-iodo-2,7-naphthyridin-1(2H)-one (Preparation 145, 6 g, 19.5 mmol), isopropenylboronic acid pinacol ester (4.9 g, 29.2 mmol), K₂CO₃ (5.46 g, 39 mmol) and Pd(amphos)Cl₂ (1.37 g, 1.95 mmol) in DMF/water (500 mL/100 mL) was heated to 80° C. for 8 h under N₂. The cooled reaction was filtered, the filtrate extracted with EtOAc (3×200 mL), the organic layers combined and dried over anhydrous Na₂SO₄ and concentrated in vacuo. The residue was purified by column chromatography on silica gel eluting with EtOAc/PE (1:10 to 1:1) to give the title compound, 1.8 g, 42%, as a light brown solid. LCMS m/z=221 [M+H]⁺

### Preparation 253

### 6-chloro-4-isopropyl-2,7-naphthyridin-1(2H)-one

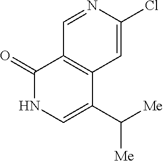 [SMILES: CC(C)c1c[nH]c(=O)c2cnc(Cl)cc12]

A mixture of 6-chloro-4-(prop-1-en-2-yl)-2,7-naphthyridin-1(2H)-one (Preparation 252, 1.8 g, 8.15 mmol) and PtO₂ (1.85 g, 8.15 mmol) in EtOAc (50 mL) was stirred at rt for 1.5 h under a H₂ atmosphere. The reaction was filtered and the filtrate concentrated in vacuo to give the title compound, 1.7 g, 94%, as a light brown solid. LCMS m/z=223 [M+H]⁺

### Preparation 254

### 6-chloro-4-isopropyl-2,7-naphthyridin-1-yl trifluoromethanesulfonate

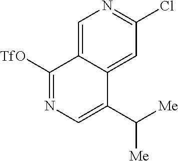 [SMILES: CC(C)c1cnc(OS(=O)(=O)C(F)(F)F)c2cnc(Cl)cc12]

To a solution of 6-chloro-4-isopropyl-2,7-naphthyridin-1(2H)-one (Preparation 253, 50 mg, 0.224 mmol) and TEA (120 mg, 1.2 mmol) in DCM (4 mL) was added dropwise, Tf₂O (313 mg, 1.11 mmol) in DCM (1 mL) at −78° C. under N₂ and the reaction stirred for 30 min. The reaction was slowly warmed to rt and stirred for 1 h. The reaction mixture was quenched with water and extracted with DCM (3×20 mL). The combined organic layers were dried over Na₂SO₄ and evaporated under reduced pressure to give the title compound as a brown oil. LCMS m/z=355 [M+H]⁺

### Preparation 255

### 6-chloro-1-iodo-4-isopropyl-2,7-naphthyridine

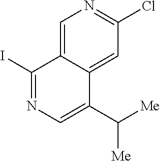 [SMILES: CC(C)c1cnc(I)c2cnc(Cl)cc12]

Trifluoromethanesulfonic acid (126 mg, 0.843 mmol) was added to a solution of 6-chloro-4-isopropyl-2,7-naphthyridin-1-yl trifluoromethanesulfonate (Preparation 254, 100 mg, 0.281 mmol) and NaI (84 mg, 0.532 mmol) in MeCN (4 mL) at −10° C. The solution was stirred at this temperature for 20 min, then allowed to warm to rt and stirred for a further 2 h. Water was added and the reaction was extracted with EtOAc (20 mL×2), the combined organic layers dried over anhydrous Na₂SO₄ and concentrated in vacuo. The crude product was purified by prep TLC (EtOAc:PE=1:1) to give the title compound, 30 mg, 32% as a light yellow solid. LCMS m/z=333 [M+H]⁺

### Preparation 256

### 2-(6-chloro-4-isopropyl-2,7-naphthyridin-1-yl)-5-methyl-1,3,4-oxadiazole

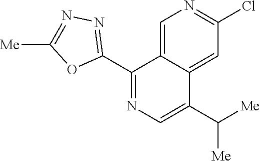 [SMILES: Cc1nnc(-c2ncc(C(C)C)c3cc(Cl)ncc23)o1]

A mixture of 2-methyl-1,3,4-oxadiazole (84.0 mg, 1 mmol), 6-chloro-1-iodo-4-isopropyl-2,7-naphthyridine (Preparation 255, 166 mg, 0.50 mol), CuI (9.60 mg, 0.05 mmol), 1,10-phenanthroline (13.4 mg, 0.075 mmol) and tBuOLi (60.0 mg, 0.75 mmol) in DMF (5 mL) was heated at 130° C. for 18 h. After cooling to rt, the mixture was concentrated in vacuo and purified by prep-TLC (PE/EtOAc=1:1) to afford the title compound, 80 mg, 57.1% as a yellow solid. ¹HNMR (300 MHz, DMSO-d₆) δ: 10.37 (d, 1H), 8.88 (s, 1H), 8.35 (d, 1H), 3.78 (p, 1H), 1.39 (d, 6H)

### Preparation 257

### 6-chloro-4-isopropyl-1-(5-methyl-4-((2-(trimethylsilyl)ethoxy)methyl)-4H-1,2,4-triazol-3-yl)-2,7-naphthyridine

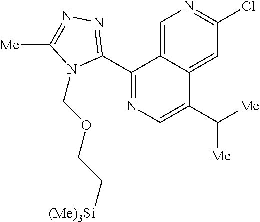

The title compound was obtained as a light yellow solid, 12 mg, 13.4% yield, from 6-chloro-1-iodo-4-isopropyl-2,7-naphthyridine (Preparation 255) and 3-methyl-1-((2-(trimethylsilyl)ethoxy)methyl)-1H-1,2,4-triazole (Preparation 143), following a similar procedure to that described in Preparation 144. LCMS m/z=418 [M+H]⁺

### Preparation 258

### 6-chloro-4-isopropyl-1-(1-((2-(trimethylsilyl)ethoxy)methyl)-1H-1,2,4-triazol-5-yl)-2,7-naphthyridine

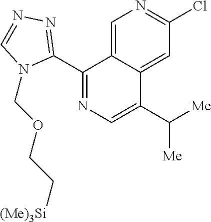

The title compound was obtained as a yellow oil, 120 mg, 30% yield, from 6-chloro-1-iodo-4-isopropyl-2,7-naphthyridine (Preparation 255) and 1-((2-(trimethylsilyl)ethoxy)methyl)-1H-1,2,4-triazole, following a similar procedure to that described in Preparation 144.

### Preparation 259

### 6-chloro-4-isopropyl-1-(1H-1,2,4-triazol-3-yl)-2,7-naphthyridine

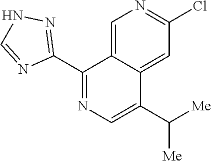 [SMILES: CC(C)c1cnc(-c2nc[nH]n2)c2cnc(Cl)cc12]

A solution of 6-chloro-4-isopropyl-1-(1-((2-(trimethylsilyl)ethoxy)methyl)-1H-1,2,4-triazol-5-yl)-2,7-naphthyridine (Preparation 258, 60.5 mg, 0.150 mmol) in HCl (1 mL, 4.0 mmol, 4M in dioxane) was stirred at rt for 1 h. The mixture was neutralized with sat. NaHCO₃ solution and extracted with EtOAc (10 mL×3). The combined organic layers were concentrated in vacuo and the residue purified by prep-TLC (DCM/MeOH=20:1) to afford the title compound (28 mg, 68.3%) as a white solid.

### Preparation 260

### 6-chloro-4-isopropyl-1-(1-methyl-1H-1,2,4-triazol-3-yl)-2,7-naphthyridine

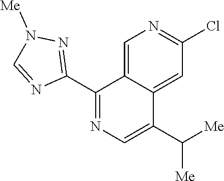 [SMILES: CC(C)c1cnc(-c2ncn(C)n2)c2cnc(Cl)cc12]

To a solution of 6-chloro-4-isopropyl-1-(1H-1,2,4-triazol-3-yl)-2,7-naphthyridine (Preparation 259, 27.3 mg, 0.10 mmol) in THF (1 mL) was added NaH (4.80 mg, 0.12 mmol) at 0° C. and the solution stirred for 1 h. Mel (28.4 mg, 0.20 mmol) was added and the reaction stirred at rt for 1 h. The reaction was quenched with brine (20 mL), extracted with EtOAc (5 mL×3) and the combined organic layers concentrated in vacuo. The residue was purified by prep-TLC (DCM/MeOH=20:1) to afford the title compound (20 mg, 69.7%) as a white solid.

### Preparation 261

### 6-chloro-4-isopropyl-1-(1-methyl-1H-pyrazol-4-yl)-2,7-naphthyridine

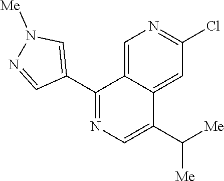 [SMILES: CC(C)c1cnc(-c2cnn(C)c2)c2cnc(Cl)cc12]

To a solution of 6-chloro-4-isopropyl-2,7-naphthyridin-1-yl trifluoromethanesulfonate (Preparation 254, 300 mg, 0.845 mmol), 1-methyl-4-(4,4,5,5-tetramethyl-1,3,2-dioxaborolan-2-yl)-1H-pyrazole (175 mg, 0.845 mmol), K₂CO₃ (236 mg, 1.69 mmol) and Pd(dppf)Cl₂ (69 mg, 0.085 mmol) in dioxane/water (8 mL/2 mL) was heated at 60° C. for 1 h under N₂. The cooled reaction was concentrated in vacuo and purified by prep TLC (PE:EtOAc=2:1) to give the title compound, 90 mg, 37.1%, as a light yellow solid. LCMS m/z=287 [M+H]⁺

### Preparation 262

### 6-chloro-4-isopropyl-1-(1-methyl-1H-1,2,3-triazol-4-yl)-2,7-naphthyridine

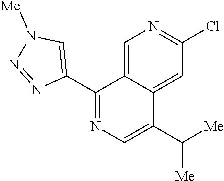 [SMILES: CC(C)c1cnc(-c2cn(C)nn2)c2cnc(Cl)cc12]

A mixture of (1-methyl-1H-1,2,3-triazol-4-yl)boronic acid (200 mg, 1.576 mmol), 6-chloro-4-isopropyl-2,7-naphthyridin-1-yl trifluoromethanesulfonate (Preparation 254, 559.0 mg, 1.576 mmol), K₂CO₃ (438.78 mg, 3.152 mmol) and Pd(dppf)Cl₂ (115.31 mg, 0.158 mmol) in dioxane (5 mL)/water (1 mL) was heated at 90° C. under N₂ in a sealed vessel for 2 h. The mixture was cooled to rt and the solids were filtered off. The filtrate was extracted with EtOAc (3×5 mL) and the combined organic extracts concentrated in vacuo. The residue was purified by silica gel column (EtOAc/PE (3:1)) to give the title compound, 100 mg, 22.1% as a white solid. LCMS m/z=288 [M+H]⁺

### Preparation 263

### (5-((dimethylamino)methyl)-1-methyl-1H-pyrazol-4-yl)boronic acid

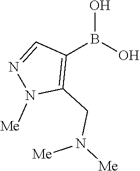 [SMILES: CN(C)Cc1c(B(O)O)cnn1C]

n-BuLi (2M in hexane, 3.8 mmol) was added dropwise to a solution of 1-(4-bromo-1-methyl-1H-pyrazol-5-yl)-N,N-dimethylmethanamine (WO2011135376, Intermediate 130, 550 mg, 2.53 mmol) in THF at −78° C. under N₂ and the reaction stirred for 0.5 h at −78° C. 4,4,5,5-Tetramethyl-2-(propan-2-yloxy)-1,3,2-dioxaborolane (990 mg, 5.0 mmol) was added and the reaction was slowly warmed to rt. The mixture was quenched with water, extracted with EtOAc and the combined organic extracts evaporated under reduced pressure to give the title compound, 510 mg, crude.

### Preparation 264

### 1-(4-(6-chloro-4-isopropyl-2,7-naphthyridin-1-yl)-1-methyl-1H-pyrazol-5-yl)-N,N-dimethylmethanamine

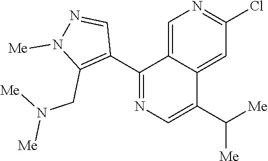 [SMILES: CC(C)c1cnc(-c2cnn(C)c2CN(C)C)c2cnc(Cl)cc12]

A mixture of (5-((dimethylamino)methyl)-1-methyl-1H-pyrazol-4-yl)boronic acid (Preparation 263, 510 mg, crude), 1,6-dichloro-4-isopropyl-2,7-naphthyridine (Preparation 148, 607 mg, 2.53 mmol), Pd(dppf)Cl₂ (184 mg, 0.25 mmol) and K₂CO₃ (398 mg, 5.06 mmol) in dioxane/H₂O (20 mL/8 mL) was stirred for 2 h at 60° C. in a sealed vessel. The mixture was concentrated in vacuo and the residue extracted with EtOAc. The organic layer was concentrated in vacuo and the residue was purified by prep-TLC with DCM/MeOH (20:1) to give the title compound, 160 mg, 18.3%, as a light yellow solid. LCMS m/z=344 [M+H]⁺

### Preparation 265

### 6-chloro-1-(1-(difluoromethyl)-1H-pyrazol-4-yl)-4-isopropyl-2,7-naphthyridine

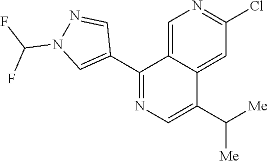 [SMILES: CC(C)c1cnc(-c2cnn(C(F)F)c2)c2cnc(Cl)cc12]

The title compound was obtained as a yellow solid, 150 mg, 74.7% yield, from 1,6-dichloro-4-isopropyl-2,7-naphthyridine (Preparation 148) and 1-(difluoromethyl)-4-(4,4,5,5-tetramethyl-1,3,2-dioxaborolan-2-yl)-1H-pyrazole, following a similar procedure to that described in Preparation 264. LCMS m/z=323 [M+H]⁺

### Preparation 266

### 6-chloro-1-(1,5-dimethyl-1H-1,2,3-triazol-4-yl)-4-isopropyl-2,7-naphthyridine

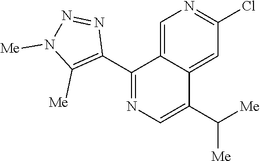 [SMILES: Cc1c(-c2ncc(C(C)C)c3cc(Cl)ncc23)nnn1C]

The title compound was obtained, 130 mg, 69.2% yield, from 1,6-dichloro-4-isopropyl-2,7-naphthyridine (Preparation 148) and 1,5-dimethyl-4-(4,4,5,5-tetramethyl-1,3,2-dioxaborolan-2-yl)-1H-1,2,3-triazole, following a similar procedure to that described in Preparation 264. LCMS m/z=302 [M+H]⁺

### Preparation 267

### 6-chloro-4-isopropyl-1-(1-(methylsulfonyl)-1H-pyrazol-4-yl)-2,7-naphthyridine

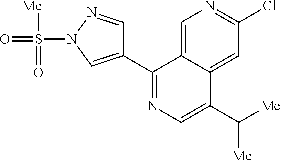 [SMILES: CC(C)c1cnc(-c2cnn(S(C)(=O)=O)c2)c2cnc(Cl)cc12]

The title compound was obtained, 120 mg, 93.2% yield, from 1,6-dichloro-4-isopropyl-2,7-naphthyridine (Preparation 148) and 1-(methylsulfonyl)-4-(4,4,5,5-tetramethyl-1,3,2-dioxaborolan-2-yl)-1H-pyrazole, following a similar procedure to that described in Preparation 264. LCMS m/z=351 [M+H]⁺

### Preparation 268

### 4-iodo-1-(oxetan-3-yl)-1H-imidazole

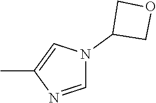

To a solution of 4-iodo-1H-imidazole (1.00 g, 5.16 mmol) in DMF (10 mL) was added NaH (412 mg, 10.3 mmol, 60% purity) at 0° C., and the reaction mixture was stirred at 20° C. for 0.5 h under N₂. The mixture was cooled to 0° C., 3-iodooxetane (1.04 g, 5.67 mmol) added and the reaction mixture stirred at 100° C. for 16 h. The mixture was poured into water (150 mL), extracted with EtOAc (60 mL×3), the combined organic layers were washed with brine (100 mL), then dried and concentrated in vacuo. The residue was purified by HPLC-26 to give the title compound, 350 mg (27%) as a white solid. ¹H NMR (400 MHz, CDCl₃) δ: 7.53 (s, 1H), 7.39 (d, 1H), 5.31-5.24 (m, 1H), 5.13-5.08 (m, 2H), 4.84-4.80 (m, 2H), 1.67 (s, 1H).

### Preparation 269

### 6-chloro-4-isopropyl-1-(1-(oxetan-3-yl)-1H-imidazol-4-yl)-2,7-naphthyridine

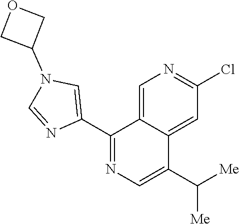 [SMILES: CC(C)c1cnc(-c2cn(C3COC3)cn2)c2cnc(Cl)cc12]

To a solution of 4-iodo-1-(oxetan-3-yl)imidazole (Preparation 268, 80.0 mg, 0.320 mmol), (BPin)₂ (244 mg, 0.960 mmol) and 1,6-dichloro-4-isopropyl-2,7-naphthyridine (Preparation 148, 77.2 mg, 0.320 mmol) in dioxane (2 mL) and H₂O (0.5 mL) was added K₂CO₃ (133 mg, 0.96 mmol) and Pd(dppf)Cl₂ (23.4 mg, 0.032 mmol) under N₂ and the reaction stirred at 100° C. for 16 h. The cooled reaction mixture was concentrated under reduced pressure and the residue was purified by silica gel chromatography (EtOAc) to give the title compound, 30.0 mg, 29% as a yellow solid.

¹H NMR (400 MHz, CDCl₃) δ: 10.74 (s, 1H), 8.61 (s, 1H), 8.15 (s, 1H), 7.87 (s, 1H), 7.80 (s, 1H), 5.45-5.37 (m, 1H), 5.22-5.15 (m, 2H), 5.02-4.96 (m, 2H), 3.60-3.47 (m, 1H), 1.46 (d, 6H)

### Preparation 270

### 6-chloro-4-isopropyl-1-(1-(oxetan-3-yl)-1H-pyrazol-3-yl)-2,7-naphthyridine

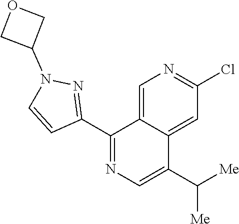 [SMILES: CC(C)c1cnc(-c2ccn(C3COC3)n2)c2cnc(Cl)cc12]

The title compound was obtained as a yellow solid, 30 mg, 29% yield, from 1,6-dichloro-4-isopropyl-2,7-naphthyridine (Preparation 148) and 3-iodo-1-(oxetan-3-yl)-1H-pyrazole (Example, 7.53, WO2015017610), following a similar procedure to that described in Preparation 269.

### Preparation 271

### (1-(1-(tert-butoxycarbonyl)azetidin-3-yl)-1H-pyrazol-4-yl)boronic acid

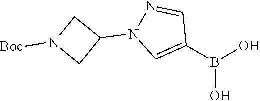 [SMILES: CC(C)(C)OC(=O)N1CC(n2cc(B(O)O)cn2)C1]

To a solution of 4-(4,4,5,5-tetramethyl-1,3,2-dioxaborolan-2-yl)-1H-pyrazole (3.00 g, 15.46 mmol) in MeCN (30 mL) was added tert-butyl 3-iodoazetidine-1-carboxylate (4.81 g, 17.01 mmol) and Cs₂CO₃ (7.56 g, 23.19 mmol) and the reaction stirred at 90° C. for 12 h. The reaction mixture was concentrated under reduced pressure to give a residue which was purified by column chromatography (PE/EtOAc=6:1 to 3:1). The product was further purified by HPLC-25 to give the title compound, 400 mg, pure) and 2.00 g, crude as a white solid. ¹H NMR (400 MHz, MeOD-d₄) δ: 7.99 (s, 1H), 7.85 (s, 1H), 5.28-5.22 (m, 1H), 4.41-4.37 (m, 2H), 4.30 (br s, 2H), 1.49 (s, 9H)

### Preparation 272

### tert-butyl 3-(4-(6-chloro-4-isopropyl-2,7-naphthyridin-1-yl)-1H-pyrazol-1-yl)azetidine-1-carboxylate

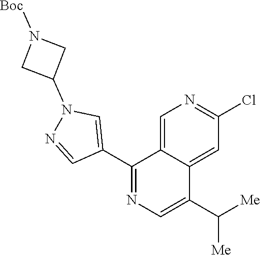 [SMILES: CC(C)c1cnc(-c2cnn(C3CN(C(=O)OC(C)(C)C)C3)c2)c2cnc(Cl)cc12]

To a solution of (1-(1-(tert-butoxycarbonyl)azetidin-3-yl)-1H-pyrazol-4-yl)boronic acid (Preparation 271, 200 mg, 0.749 mmol) in dioxane (10 mL) and H₂O (1 mL) was added K₃PO₄ (317.9 mg, 1.50 mmol), 1,6-dichloro-4-isopropyl-2,7-naphthyridine (Preparation 148, 216.7 mg, 0.90 mmol) and Pd(dppf)Cl₂ (54.79 mg, 0.075 mmol) and the reaction stirred at 50° C. for 12 h under N₂. Further (1-(1-(tert-butoxycarbonyl)azetidin-3-yl)-1H-pyrazol-4-yl)boronic acid (70.0 mg), K₃PO₄ (200 mg) and Pd(dppf)Cl₂ (50 mg) were added and the reaction mixture was stirred at 50° C. for a further 12 h. The reaction mixture was concentrated under reduced pressure and the residue was purified by prep-TLC (EtOAc) to give the title compound, 100 mg, crude as a yellow oil.

### Preparation 273

### 6-chloro-4-isopropyl-1-(1-(1-methylazetidin-3-yl)-1H-pyrazol-4-yl)-2,7-naphthyridine formate

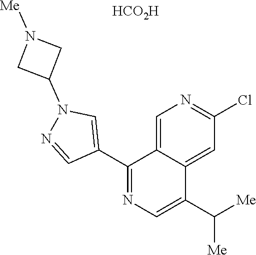 [SMILES: CC(C)c1cnc(-c2cnn(C3CN(C)C3)c2)c2cnc(Cl)cc12 ; "HCO2H"]

Part A: A mixture of tert-butyl 3-(4-(6-chloro-4-isopropyl-2,7-naphthyridin-1-yl)-1H-pyrazol-1-yl)azetidine-1-carboxylate (Preparation 272, 90.0 mg, 0.21 mmol) in DCM (3 mL) and TFA (1 mL) was stirred at 15° C. for 30 min. The reaction mixture was concentrated under reduced pressure to give 1-(1-(azetidin-3-yl)-1H-pyrazol-4-yl)-6-chloro-4-isopropyl-2,7-naphthyridine trifluoroacetate, 65.0 mg, crude as a brown oil.

Part B: To a solution of 1-(1-(azetidin-3-yl)-1H-pyrazol-4-yl)-6-chloro-4-isopropyl-2,7-naphthyridine trifluoroacetate (55.0 mg, 0.168 mmol) in MeOH (1.00 mL) was added HCHO (27.23 mg, 0.336 mmol) and the reaction stirred at 15° C. for 2 h. NaBH(OAc)₃ (71.12 mg, 0.336 mmol) was added and the reaction stirred at 15° C. for 2 h. The reaction mixture was concentrated under reduced pressure and the residue was purified by HPLC-29 to give the title compound, 60.0 mg, 92.2%, as a yellow oil.

### Preparation 274

### methyl 6-chloro-4-isopropyl-2,7-naphthyridine-1-carboxylate

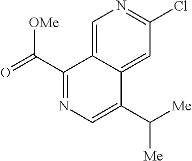 [SMILES: COC(=O)c1ncc(C(C)C)c2cc(Cl)ncc12]

A mixture of 6-chloro-4-isopropyl-2,7-naphthyridin-1-yl trifluoromethanesulfonate (Preparation 254, 220 mg, 0.62 mmol) was dissolved in MeOH (10 mL), TEA (250 mg, 2.48 mmol), Pd(dppf)Cl₂ (51 mg, 0.062 mmol) were added and the reaction heated to 50° C. under CO (5 atm) for 2 h. The reaction mixture was concentrated in vacuo and purified by TLC (1/1 PE/EtOAc) to give the title compound, 100 mg, 61.1%, as an off-white solid. LCMS m/z=265 [M+H]⁺

### Preparation 275

### 1-(2-(6-chloro-4-isopropyl-2,7-naphthyridine-1-carbonyl)hydrazineyl)-2-methyl-1-oxopropan-2-yl acetate

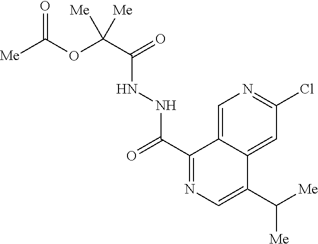 [SMILES: CC(=O)OC(C)(C)C(=O)NNC(=O)c1ncc(C(C)C)c2cc(Cl)ncc12]

Part A: A solution of methyl 6-chloro-4-isopropyl-2,7-naphthyridine-1-carboxylate (Preparation 274, 300 mg, 1.13 mmol) and hydrazine hydrate (113 mg, 2.26 mmol) in MeOH (5 mL) was heated at 70° C. for 2 h. The cooled reaction was concentrated in vacuo and purified by prep TLC (PE:EtOAc=2:1) to give 6-chloro-4-isopropyl-2,7-naphthyridine-1-carbohydrazide, 300 mg as a light yellow solid.

Part B: 1-Chloro-2-methyl-1-oxopropan-2-yl acetate (130 mg, 0.622 mmol) was added to a solution of 6-chloro-4-isopropyl-2,7-naphthyridine-1-carbohydrazide (150 mg, 0.566 mmol) and TEA (114 mg, 1.13 mmol) in DCM (5 mL) in an ice bath and the reaction mixture was stirred at rt for 2 h. The reaction was partitioned between DCM (20 mL) and water (15 mL), the layers separated and the aqueous extracted with DCM (20 mL). The combined organic extracts were dried over anhydrous Na₂SO₄ and concentrated in vacuo. The crude product was purified by prep TLC (PE:EtOAc=3:1) to give the title compound, 120 mg, 53.9% as light yellow oil. LCMS m/z=393 [M+H]⁺

### Preparation 276

### 2-(5-(6-chloro-4-isopropyl-2,7-naphthyridin-1-yl)-1,3,4-oxadiazol-2-yl)propan-2-yl acetate

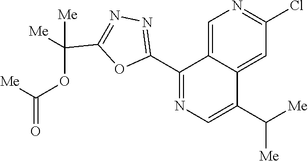 [SMILES: CC(=O)OC(C)(C)c1nnc(-c2ncc(C(C)C)c3cc(Cl)ncc23)o1]

TsCl (237 mg, 1.25 mmol) was added to 1-(2-(6-chloro-4-isopropyl-2,7-naphthyridine-1-carbonyl)hydrazineyl)-2-methyl-1-oxopropan-2-yl acetate (Preparation 275, 450 mg, 1.14 mmol) and TEA (230 mg, 2.28 mmol) in DCM (10 mL) and the reaction stirred at rt for 12 h. The reaction mixture was concentrated in vacuo and the residue purified by column chromatography with PE: EtOAc=3:1 to give the title compound, 400 mg, 93.6% yield, as a white solid. LCMS m/z=375 [M+H]⁺

### Preparation 277

### 6-chloro-4-isopropyl-1-((1-methyl-1H-pyrazol-4-yl)ethynyl)-2,7-naphthyridine

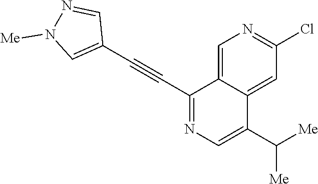 [SMILES: CC(C)c1cnc(C#Cc2cnn(C)c2)c2cnc(Cl)cc12]

A mixture of 1,6-dichloro-4-isopropyl-2,7-naphthyridine (Preparation 148, 150 mg, 0.622 mmol), 4-ethynyl-1-methyl-1H-pyrazole (66.0 mg, 0.622 mmol), CuI (35.4 mg, 0.186 mmol), Pd(PPh₃)₂Cl₂ (43.5 mg, 0.062 mmol) and TEA (250 mg, 2.48 mmol) in THF (20 mL) was stirred at 70° C. for 16 h. The reaction mixture was concentrated in vacuo and the residue purified by prep-TLC (DCM:MeOH=30:1) to afford the title compound, 140 mg, 72.4%, as a yellow solid. LCMS m/z=311 [M+H]⁺

### Preparation 278

### 6-chloro-1-(3-fluoroazetidin-1-yl)-4-isopropyl-2,7-naphthyridine

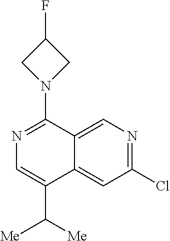 [SMILES: CC(C)c1cnc(N2CC(F)C2)c2cnc(Cl)cc12]

A solution of 6-chloro-4-isopropyl-2,7-naphthyridin-1(2H)-one (Preparation 253, 227.7 mg, 1 mmol), 3-fluoroazetidine (187.7 mg, 2.5 mmol) and DBU (563.3 mg, 3.7 mmol) in DMF (5 mL) was cooled in an ice-bath, the mixture stirred for 10 mins, then BOP (531 mg, 1.2 mmol) added, dropwise. The reaction was stirred at 25° C. for 4 h then diluted with water and extracted with EtOAc. The combined organic extracts were dried over Na₂SO₄, filtered and concentrated in vacuo. The crude product was purified by silica gel column eluting with MeOH-DCM (1:50) to afford the title compound, 125 mg, 44.6%, as a white solid. LCMS m/z=280 [M+H]⁺

### Preparation 279

### 6-chloro-4-isopropyl-1-(methylsulfonyl)-2,7-naphthyridine

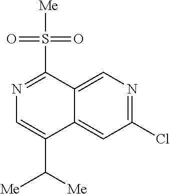 [SMILES: CC(C)c1cnc(S(C)(=O)=O)c2cnc(Cl)cc12]

Sodium methanesulfinate (178 mg, 1.74 mmol) and 1,6-dichloro-4-isopropyl-2,7-naphthyridine (Preparation 148, 400 mg, 1.659 mmol) in dimethylacetamide (5.53 mL) was stirred at rt. The reaction mixture was concentrated in vacuo and the residue purified by column chromatography to give the title compound.

### Preparation 280

### (3S,4R)-3-fluoro-1-(4-((5-isopropyl-8-(methylsulfonyl)-2,7-naphthyridin-3-yl)amino)pyrimidin-2-yl)-3-methylpiperidin-4-ol

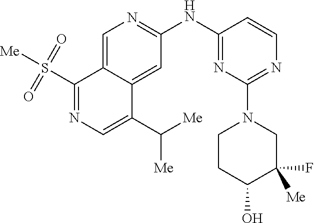 [SMILES: CC(C)c1cnc(S(C)(=O)=O)c2cnc(Nc3ccnc(N4CC[C@@H](O)[C@@](C)(F)C4)n3)cc12]

The title compound was obtained from 6-chloro-4-isopropyl-1-(methylsulfonyl)-2,7-naphthyridine (Preparation 279) and (3S,4R)-1-(4-aminopyrimidin-2-yl)-3-fluoro-3-methylpiperidin-4-ol (Preparation 8) following a similar procedure to that described in Example 162, part 1. LCMS m/z=475 [M+H]⁺

### Preparation 281

### 1,6-dichloro-4-methyl-2,7-naphthyridine

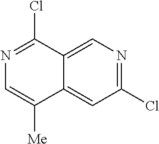 [SMILES: Cc1cnc(Cl)c2cnc(Cl)cc12]

A mixture of 1,6-dichloro-4-iodo-2,7-naphthyridine (Preparation 146, 3 g, 9.23 mmol), 2,4,6-trimethyl-1,3,5,2,4,6-trioxatriborinane (1.38 g, 11.0 mmol), K₂CO₃ (1.90 g, 13.8 mmol) and Pd(dppf)Cl₂·DCM (1.12 g, 1.38 mmol) in dioxane (30 mL) and H₂O (5 mL) was stirred at 85° C. for 8 h under N₂. The cooled mixture was diluted with EtOAc (50 mL) and washed with brine (30 mL×2). The organic layer was dried over Na₂SO₄ and concentrated in vacuo. The residue was purified by column chromatography on silica gel (PE:EtOAc=10:1 to 3:1) to give the title compound, 0.7 g 35.7% as a white solid. LCMS m/z=213 [M+H]⁺

### Preparation 282

### 4-(bromomethyl)-1,6-dichloro-2,7-naphthyridine

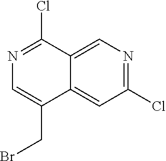 [SMILES: Clc1cc2c(CBr)cnc(Cl)c2cn1]

AIBN (53.8 mg, 0.328 mmol) was added to a solution of 1,6-dichloro-4-methyl-2,7-naphthyridine (Preparation 281, 0.7 g, 3.28 mmol) and NBS (699 mg, 3.93 mmol) in DCE (10 mL), was added and the reaction stirred at 80° C. for 3 h. The cooled mixture was diluted with EtOAc (20 mL) and washed with brine (10 mL×2). The organic layer was dried over Na₂SO₄ and concentrated in vacuo. The residue was purified by column chromatography on silica gel (PE:EtOAc=10:1 to 3:1) to give the title compound, 0.71 g, 74.2% as a light yellow solid. LCMS m/z=293 [M+H]⁺

### Preparation 283

### dimethyl 2-((1,6-dichloro-2,7-naphthyridin-4-yl)methyl)-2-methylmalonate

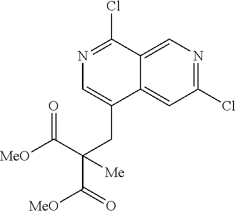 [SMILES: COC(=O)C(C)(Cc1cnc(Cl)c2cnc(Cl)cc12)C(=O)OC]

NaH (114 mg, 2.86 mmol, 60%) was added to an ice cooled solution of 1,3-dimethyl 2-methylpropanedioate (349 mg, 2.39 mmol) in THF (10 mL), and the solution stirred at 0° C. for 0.5 h. 4-(Bromomethyl)-1,6-dichloro-2,7-naphthyridine (Preparation 282, 0.7 g, 2.39 mmol) was added and the resulting solution stirred at 25° C. for 2 h. The mixture was poured into 0.5 N a.q. HCl (10 mL), diluted with EtOAc (20 mL×3) and washed with brine (10 mL). The organic layer was dried over Na₂SO₄ and concentrated in vacuo. The residue was purified by column chromatography on silica gel (PE:EtOAc=20:1 to 3:1) to give the title compound 0.68 g, 79.2% as a light yellow solid. LCMS m/z=357 [M+H]⁺

### Preparation 284

### dimethyl 2-((6-chloro-1-(2-methylazetidin-1-yl)-2,7-naphthyridin-4-yl)methyl)-2-methylmalonate

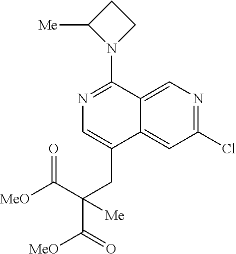 [SMILES: COC(=O)C(C)(Cc1cnc(N2CCC2C)c2cnc(Cl)cc12)C(=O)OC]

TEA (0.652 g, 3.82 mmol) was added to a solution of dimethyl 2-((1,6-dichloro-2,7-naphthyridin-4-yl)methyl)-2-methylmalonate (Preparation 283, 0.652 g, 1.82 mmol) and 2-methylazetidine hydrochloride (215 mg, 2.0 mmol) in IPA (10 mL) and the reaction was heated at 90° C. for 5 h. The cooled mixture was diluted with EtOAc (20 mL×3) and washed with brine (10 mL). The organic layer was dried over Na₂SO₄, concentrated in vacuo and the residue was purified by silica gel chromatography (PE:EtOAc=10:1 to 3:2) to give the title compound, 0.70 g, 98.1% as a light yellow gum. LCMS m/z=392 [M+H]⁺

### Preparation 285

### 2-((6-chloro-1-(2-methylazetidin-1-yl)-2,7-naphthyridin-4-yl)methyl)-2-methylpropane-1,3-diol

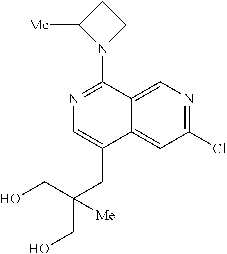 [SMILES: CC1CCN1c1ncc(CC(C)(CO)CO)c2cc(Cl)ncc12]

LiAlH₄ (94.1 mg, 2.48 mmol) was added to an ice cooled solution of dimethyl 2-((6-chloro-1-(2-methylazetidin-1-yl)-2,7-naphthyridin-4-yl)methyl)-2-methylmalonate (Preparation 284, 243 mg, 0.62 mmol) in THF (15 mL) and the reaction was stirred at 25° C. for 10 h. The mixture was quenched sequentially with H₂O (0.1 mL), 15% a.q. NaOH (0.1 mL), H₂O (0.3 mL) and then Na₂SO₄ (5 g) added. The mixture was filtered, washed with EtOAc (50 mL×3) and the filtrate concentrated in vacuo. The residue was purified by prep. TLC (PE:EtOAc=1:10) to give the title compound, 65 mg, 31.3% as a light yellow solid. LCMS m/z=336 [M+H]⁺

### Preparation 286

### 6-chloro-1-(2-methylazetidin-1-yl)-4-((3-methyloxetan-3-yl)methyl)-2,7-naphthyridine

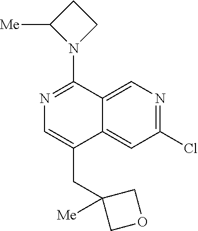 [SMILES: CC1CCN1c1ncc(CC2(C)COC2)c2cc(Cl)ncc12]

A mixture of 2-((6-chloro-1-(2-methylazetidin-1-yl)-2,7-naphthyridin-4-yl)methyl)-2-methylpropane-1,3-diol (Preparation 285, 65 mg, 0.193 mmol) and (tributylphosphoranylidene)acetonitrile (139 mg, 0.579 mmol) in toluene (10 mL) was stirred at 120° C. for 8 h. H₂O (5 mL) was added to the cooled reaction and the mixture extracted with EtOAc (20 mL×3). The combined organic extracts were washed with brine (10 mL), dried and concentrated in vacuo. The residue was purified by prep. TLC (PE:EtOAc=1:10) to give the title compound, 45 mg, 73.4% as a light yellow gum. LCMS m/z=318 [M+H]⁺

### Preparation 287

### 6-chloro-4-iodo-1-(2-methylazetidin-1-yl)-2,7-naphthyridine

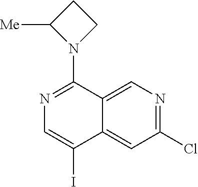 [SMILES: CC1CCN1c1ncc(I)c2cc(Cl)ncc12]

A mixture of 1,6-dichloro-4-iodo-2,7-naphthyridine (Preparation 146, 760 mg, 2.34 mmol), 2-methylazetidine hydrochloride (302 mg, 2.81 mmol) and DIPEA (1.02 mL, 5.85 mmol) in IPA (6 mL) was stirred at 90° C. overnight. The cooled mixture was diluted with water, the mixture stirred for 15 mins, then filtered and dried in vacuo to give the title compound, 712 mg, 84.5% as a pale yellow solid. LCMS m/z=360 [M+H]⁺

### Preparation 288

### (R)-6-chloro-4-iodo-1-(2-methylazetidin-1-yl)-2,7-naphthyridine

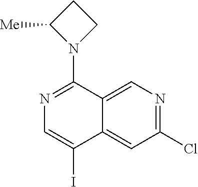 [SMILES: C[C@@H]1CCN1c1ncc(I)c2cc(Cl)ncc12]

TEA (744 mg, 7.36 mmol) was added to a solution of 1,6-dichloro-4-iodo-2,7-naphthyridine (Preparation 146, 1 g, 3.07 mmol) and (2R)-2-methylazetidine camphorsulfonate (JOC 2016, 81, 3031-3036, 1.02 g, 3.37 mmol) in IPA (18 mL) and the reaction was heated at 90° C. for 2 h. The cooled mixture was diluted with EtOAc (20 mL) and washed with brine (10 mL×2). The organic layer was dried over Na₂SO₄ and concentrated in vacuo. The residue was purified by silica gel chromatography (PE:EtOAc=10:1 to 3:2) to give the title compound, 1 g (90.9%) as a light yellow solid. LCMS m/z=360 [M+H]⁺

### Preparation 289

### (2R,3S)-1-(6-chloro-4-iodo-2,7-naphthyridin-1-yl)-2-methylazetidin-3-ol

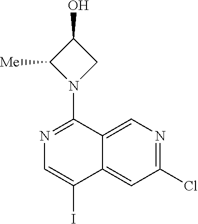 [SMILES: C[C@@H]1[C@@H](O)CN1c1ncc(I)c2cc(Cl)ncc12]

The title compound was obtained as a yellow solid, 230 mg, 66.3% yield, from 1,6-dichloro-4-iodo-2,7-naphthyridine (Preparation 146) and (2R,3S)-2-methylazetidin-3-ol trifluoroacetate (Preparation 73), following a similar procedure to that described in Preparation 288. LCMS m/z=376 [M+H]⁺

### Preparation 290

### 6-chloro-4-iodo-1-((2R,3S)-3-methoxy-2-methylazetidin-1-yl)-2,7-naphthyridine

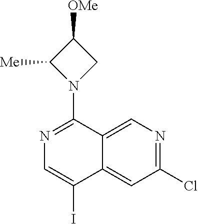 [SMILES: CO[C@H]1CN(c2ncc(I)c3cc(Cl)ncc23)[C@@H]1C]

NaH (46.0 mg, 1.15 mmol) was added to an ice-cooled solution of (2R,3S)-1-(6-chloro-4-iodo-2,7-naphthyridin-1-yl)-2-methylazetidin-3-ol (Preparation 289, 360 mg, 0.958 mmol) in DMF (5.126 mL) and the solution stirred for 5 min. Iodomethane (0.24 mL, 3.83 mmol) was added and the reaction stirred at rt for 3 days. Further NaH (30 mg) and iodomethane (0.11 mL) were added and the reaction stirred for a further 5 h. The reaction was quenched by the slow addition of water, extracted with EtOAc (3×), the combined organic extracts washed with brine, dried over Na₂SO₄ and concentrated in vacuo. The residue was purified by Isco Combiflash® to give the title compound, 320.8 mg, 86% yield as a yellow viscous oil. LCMS m/z=390 [M+H]⁺

### Preparation 291

### 6-chloro-1-((2R,3R)-3-fluoro-2-methylazetidin-1-yl)-4-iodo-2,7-naphthyridine

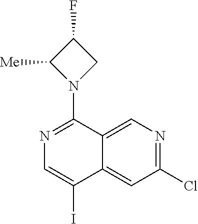 [SMILES: C[C@@H]1[C@H](F)CN1c1ncc(I)c2cc(Cl)ncc12]

DAST (188 mg, 1.17 mmol) was added to a solution of (2R,3S)-1-(6-chloro-4-iodo-2,7-naphthyridin-1-yl)-2-methylazetidin-3-ol (Preparation 289, 220 mg, 0.585 mmol) in DCM (25 mL) and the reaction stirred at rt for 2 h. The mixture was quenched with water and extracted with DCM. The organic layer was dried over Na₂SO₄, filtered and concentrated in vacuo. The residue was purified by prep-TLC (EtOAc:PE=1:10) to afford the title compound, 120 mg, 54.3% as a yellow solid. LCMS m/z=378 [M+H]⁺

### Preparation 292

### 6-chloro-1-(2-methylazetidin-1-yl)-4-vinyl-2,7-naphthyridine

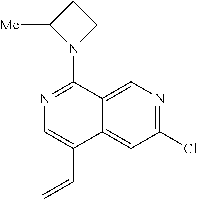 [SMILES: C=Cc1cnc(N2CCC2C)c2cnc(Cl)cc12]

A mixture of 6-chloro-4-iodo-1-(2-methylazetidin-1-yl)-2,7-naphthyridine (Preparation 287, 255 mg, 0.709 mmol), 4,4,5,5-tetramethyl-2-vinyl-1,3,2-dioxaborolane (131 mg, 0.851 mmol), K₂CO₃ (294 mg, 2.127 mmol) and Pd(amphos)Cl₂ (25 mg, 0.035 mmol) in DME (3 mL) and water (1 mL) was heated at 90° C. for 1 h under N₂. The cooled mixture was diluted with 5% MeOH/DCM and water, the layers separated and the organic layer dried over Na₂SO₄, filtered and concentrated in vacuo. The residue was purified by Isco chromatography (5 to 100% EtOAc/Hex) to give the title compound, 71 mg, 38.5% as a yellow solid. LCMS m/z=260 [M+H]⁺

### Preparation 293

### (R)-2-(6-chloro-1-(2-methylazetidin-1-yl)-2,7-naphthyridin-4-yl)prop-2-en-1-ol

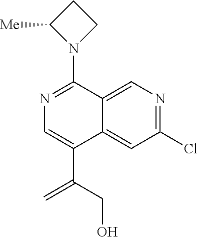 [SMILES: C=C(CO)c1cnc(N2CC[C@H]2C)c2cnc(Cl)cc12]

A mixture of (R)-4-bromo-6-chloro-1-(2-methylazetidin-1-yl)-2,7-naphthyridine (Preparation 152, 106 mg, 0.339 mmol), 2-(4,4,5,5-tetramethyl-1,3,2-dioxaborolan-2-yl)prop-2-en-1-ol (69 mg, 0.373 mmol), Na₂CO₃ (108 mg, 1.017 mmol), and Pd(PPh₃)₄ (20 mg, 0.017 mmol) in DME (0.75 mL) and water (0.25 mL) was heated at 90° C. under N₂ for 3 h. Additional 2-(4,4,5,5-tetramethyl-1,3,2-dioxaborolan-2-yl)prop-2-en-1-ol (69 mg) and Pd(PPh₃)₄ (20 mg, 0.017 mmol) were added and the reaction stirred overnight. The mixture was diluted with 5% MeOH/DCM and water, the layers separated, the organic layer was dried over Na₂SO₄, filtered and evaporated under reduced pressure. The crude was purified by Isco chromatography (0 to 10% MeOH/DCM) to give the title compound, 71 mg, 72.3%, as an off-white solid. LCMS m/z=290 [M+H]⁺

### Preparation 294

### 2-(6-chloro-1-((2R,3S)-3-methoxy-2-methylazetidin-1-yl)-2,7-naphthyridin-4-yl)prop-2-en-1-ol

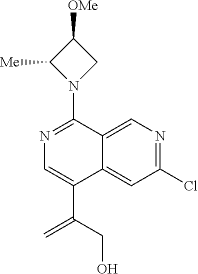 [SMILES: C=C(CO)c1cnc(N2C[C@H](OC)[C@H]2C)c2cnc(Cl)cc12]

6-Chloro-4-iodo-1-((2R,3S)-3-methoxy-2-methylazetidin-1-yl)-2,7-naphthyridine (Preparation 290, 300 mg, 0.77 mmol), K₃PO₄ (490 mg, 2.31 mmol), XPhos Pd G4 (19.88 mg, 0.023 mmol) and 2-(4,4,5,5-tetramethyl-1,3,2-dioxaborolan-2-yl)prop-2-en-1-ol (283 mg, 1.54 mmol) were dissolved in THF (2.77 mL)/water (2.77 mL) and the reaction stirred at 50° C. for 2 h. The reaction was partitioned between water and 5% MeOH in DCM, the layers separated, the organic phase dried over Na₂SO₄ and concentrated in vacuo. The residue was purified by Isco Combiflash® (0-10% MeOH in DCM) to give the title compound as an orange viscous oil, 182 mg, 73.9%. LCMS m/z=320 [M+H]⁺

### Preparation 295

### 2-(6-chloro-1-((2R,3R)-3-fluoro-2-methylazetidin-1-yl)-2,7-naphthyridin-4-yl)prop-2-en-1-ol

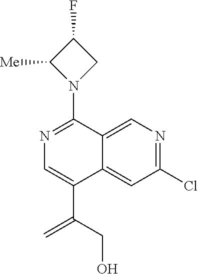 [SMILES: C=C(CO)c1cnc(N2C[C@@H](F)[C@H]2C)c2cnc(Cl)cc12]

A mixture of 6-chloro-1-((2R,3R)-3-fluoro-2-methylazetidin-1-yl)-4-iodo-2,7-naphthyridine (Preparation 291, 110 mg, 0.291 mmol), 2-(4,4,5,5-tetramethyl-1,3,2-dioxaborolan-2-yl)prop-2-en-1-ol (53.5 mg, 0.291 mmol), Pd(dppf)Cl₂·DCM (35.6 mg, 0.044 mmol) and Cs₂CO₃ (189 mg, 0.582 mmol) in dioxane (20 mL) and water (5 mL) was stirred at 90° C. for 2 h. The mixture was concentrated in vacuo and the residue was purified by prep-TLC (DCM:MeOH=30:1) to give the title compound, 50 mg, 55.8% yield, as a dark-grey oil. LCMS m/z=308 [M+H]⁺

### Preparation 296

### 6-chloro-4-ethyl-1-(2-methylazetidin-1-yl)-2,7-naphthyridine

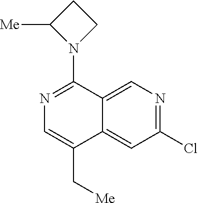 [SMILES: CCc1cnc(N2CCC2C)c2cnc(Cl)cc12]

A solution of 6-chloro-1-(2-methylazetidin-1-yl)-4-vinyl-2,7-naphthyridine (Preparation 292, 71 mg, 0.273 mmol) and PtO₂ (6.21 mg, 0.027 mmol) in MeOH (5 mL) was stirred at rt for 2 h under an atmosphere of H₂. The reaction was filtered through Celite® and the filtrate evaporated under reduced pressure to afford the title compound, 72 mg. LCMS m/z=262 [M+H]⁺

### Preparation 297

### 2-(6-chloro-1-((2R,3S)-3-methoxy-2-methylazetidin-1-yl)-2,7-naphthyridin-4-yl)propan-1-ol

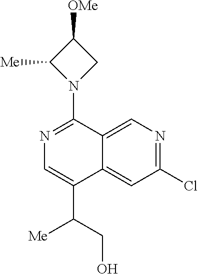 [SMILES: CO[C@H]1CN(c2ncc(C(C)CO)c3cc(Cl)ncc23)[C@@H]1C]

A solution of 2-(6-chloro-1-((2R,3S)-3-methoxy-2-methylazetidin-1-yl)-2,7-naphthyridin-4-yl)prop-2-en-1-ol (Preparation 294, 180 mg, 0.563 mmol) and PtO₂ (12.78 mg, 0.056 mmol) in MeOH (5.63 mL) was stirred at rt for 1.5 h under an atmosphere of H₂. The reaction was filtered through silica gel and the filtrate evaporated under reduced pressure to afford the title compound, 180 mg. ¹H NMR (400 MHz, DMSO-d₆) δ 9.13 (d, 1H), 8.15 (d, 1H), 7.94 (d, 1H), 4.92 (dd, 1H), 4.70 (td, 1H), 4.56 (p, 1H), 4.05-3.97 (m, 1H), 3.63-3.55 (m, 1H), 3.49 (dq, 1H), 3.29 (d, 3H), 1.48 (dd, 3H), 1.30 (dd, 3H).

### Preparation 298

### 2-(6-chloro-1-((R)-2-methylazetidin-1-yl)-2,7-naphthyridin-4-yl)propan-1-ol

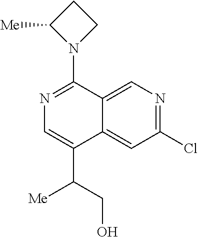 [SMILES: CC(CO)c1cnc(N2CC[C@H]2C)c2cnc(Cl)cc12]

The title compound was obtained as a brown oily residue, 68 mg, from (R)-2-(6-chloro-1-(2-methylazetidin-1-yl)-2,7-naphthyridin-4-yl)prop-2-en-1-ol (Preparation 293), following the procedure described in Preparation 297. LCMS m/z=292 [M+H]⁺

### Preparation 299

### 2-(6-chloro-1-((2R,3R)-3-fluoro-2-methylazetidin-1-yl)-2,7-naphthyridin-4-yl)propan-1-ol

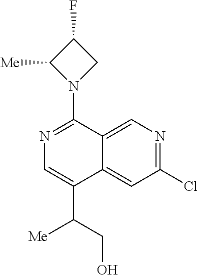 [SMILES: CC(CO)c1cnc(N2C[C@@H](F)[C@H]2C)c2cnc(Cl)cc12]

A mixture of 2-(6-chloro-1-((2R,3R)-3-fluoro-2-methylazetidin-1-yl)-2,7-naphthyridin-4-yl)prop-2-en-1-ol (Preparation 295, 100 mg, 0.324 mmol) and PtO₂ (36.7 mg, 0.162 mmol) in EtOAc (20 mL) was stirred at rt for 1 h under a H₂ atmosphere. The mixture was filtered, the filtrate was concentrated in vacuo. The residue was purified by prep-TLC (DCM:MeOH=30:1) to afford the title compound, 60 mg, 59.7%, as a light-yellow oil. LCMS m/z=310 [M+H]⁺

### Preparation 300

### 6-chloro-4-(1-methoxypropan-2-yl)-1-((R)-2-methylazetidin-1-yl)-2,7-naphthyridine

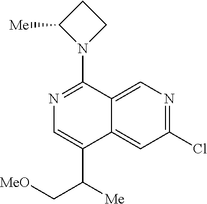 [SMILES: COCC(C)c1cnc(N2CC[C@H]2C)c2cnc(Cl)cc12]

NaH (13 mg, 0.337 mmol) was added to a solution of 2-(6-chloro-1-((R)-2-methylazetidin-1-yl)-2,7-naphthyridin-4-yl)propan-1-ol (Preparation 298, 82 mg, 0.281 mmol) and iodomethane (160 mg, 1.12 mmol) in DMF (1.5 mL) and the reaction was stirred at rt for 90 mins. The mixture was diluted with water (25 mL), extracted with EtOAc and the combined organic extracts washed with water (2×), then brine and dried over Na₂SO₄. The mixture was filtered and concentrated in vacuo and the residue purified by Isco chromatography (0 to 40% EtOAc/Hex) to give the title compound, as a pale yellow oil, 50 mg. LCMS m/z=306 [M+H]⁺

### Preparation 301

### (R)-6-chloro-1-(2-methylazetidin-1-yl)-2,7-naphthyridine-4-carbaldehyde

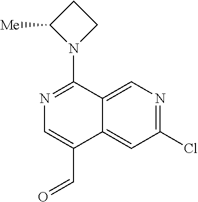 [SMILES: C[C@@H]1CCN1c1ncc(C=O)c2cc(Cl)ncc12]

A mixture of (R)-6-chloro-4-iodo-1-(2-methylazetidin-1-yl)-2,7-naphthyridine (Preparation 288, 900 mg, 2.50 mmol), triethylsilane (144 mg, 1.25 mmol), Na₂CO₃ (105 mg, 1 mmol) and Pd(dppf)Cl₂ (182 mg, 0.250 mmol) in DMF (10 mL) was stirred at 50° C. under CO (5 atm) for 3 h. The mixture was diluted with EtOAc (20 mL) and washed with brine (10 mL×2). The organic layer was dried over Na₂SO₄ and concentrated in vacuo. The residue was purified by column chromatography on silica gel (PE:EtOAc=10:1 to 3:1) to give the title compound, 500 mg, 76.4% as a brownish solid. LCMS m/z=262 [M+H]⁺

### Preparation 302

### dimethyl (R)-2-((6-chloro-1-(2-methylazetidin-1-yl)-2,7-naphthyridin-4-yl)methylene)malonate

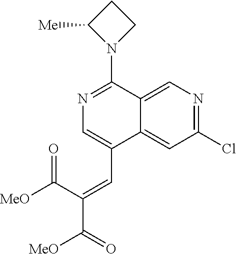 [SMILES: COC(=O)C(=Cc1cnc(N2CC[C@H]2C)c2cnc(Cl)cc12)C(=O)OC]

Titanium tetrachloride (0.67 mL, 6.08 mmol) in DCM (4 mL) was added drop wise to THF (18 mL) at 0° C. under N₂. A solution of (R)-6-chloro-1-(2-methylazetidin-1-yl)-2,7-naphthyridine-4-carbaldehyde (Preparation 301, 400 mg, 1.52 mmol) and 1,3-dimethyl propanedioate (301 mg, 2.28 mmol) in THF (8 mL) was added, followed by pyridine (0.612 mL, 7.60 mmol) and the reaction warmed to 25° C. and stirred for 10 h. The mixture was diluted with EtOAc (20 mL) and washed with brine (10 mL×2). The organic layer was dried over Na₂SO₄ and concentrated in vacuo. The residue was purified by column chromatography on silica gel (PE:EtOAc=3:1 to 3:2) to give the title compound, 400 mg (70%) as a light yellow solid. LCMS m/z=367 [M+H]⁺

### Preparation 303

### dimethyl 2-(1-(6-chloro-1-((R)-2-methylazetidin-1-yl)-2,7-naphthyridin-4-yl)ethyl)malonate

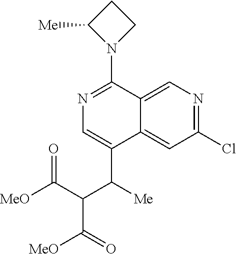 [SMILES: COC(=O)C(C(=O)OC)C(C)c1cnc(N2CC[C@H]2C)c2cnc(Cl)cc12]

Anhydrous toluene (68.88 mL) was distilled into a flask under Argon, dimethyl (R)-2-((6-chloro-1-(2-methylazetidin-1-yl)-2,7-naphthyridin-4-yl)methylene)malonate (Preparation 302, 700 mg, 1.86 mmol) added and the mixture cooled to −40° C. Dimethylaluminium chloride (6.19 mL, 5.58 mmol, 0.9 M solution in hexane) was injected via a syringe, the reaction was stirred at −40° C. for 0.5 h and warmed to 25° C. for 4 h. The mixture was diluted with EtOAc (20 mL) and washed with brine (10 mL×2). The organic layer was dried over Na₂SO₄ and concentrated in vacuo. The residue was purified by prep. TLC (PE:EtOAc=1:1) to give the title compound, 300 mg (41.2%) as a light yellow solid. LCMS m/z=392 [M+H]⁺

### Preparation 304

### 2-(1-(6-chloro-1-((R)-2-methylazetidin-1-yl)-2,7-naphthyridin-4-yl)ethyl)propane-1,3-diol

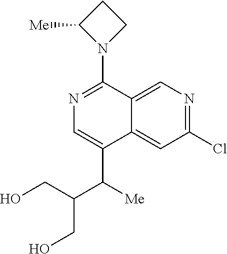 [SMILES: CC(c1cnc(N2CC[C@H]2C)c2cnc(Cl)cc12)C(CO)CO]

The title compound was obtained as a light yellow solid, 120 mg, 23.8% yield from dimethyl 2-(1-(6-chloro-1-((R)-2-methylazetidin-1-yl)-2,7-naphthyridin-4-yl)ethyl)malonate (Preparation 303), following the procedure described in Preparation 285. LCMS m/z=336 [M+H]⁺

### Preparation 305

### 6-chloro-1-((R)-2-methylazetidin-1-yl)-4-(1-(oxetan-3-yl)ethyl)-2,7-naphthyridine

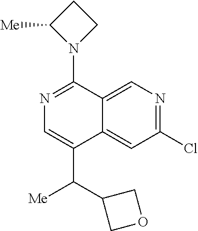 [SMILES: CC(c1cnc(N2CC[C@H]2C)c2cnc(Cl)cc12)C1COC1]

The title compound was obtained as a light yellow gum, 60 mg, 53% yield from 2-(1-(6-chloro-1-((R)-2-methylazetidin-1-yl)-2,7-naphthyridin-4-yl)ethyl)propane-1,3-diol (Preparation 304), following the procedure described in Preparation 286. LCMS m/z=318 [M+H]⁺

### Preparation 306

### 6-chloro-4-(2-fluoropyridin-3-yl)-1-(2-methylazetidin-1-yl)-2,7-naphthyridine

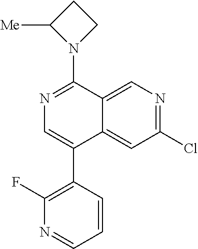 [SMILES: CC1CCN1c1ncc(-c2cccnc2F)c2cc(Cl)ncc12]

A mixture of 6-chloro-4-iodo-1-(2-methylazetidin-1-yl)-2,7-naphthyridine (Preparation 287, 88 mg, 0.245 mmol), (2-fluoropyridin-3-yl)boronic acid (41 mg, 0.294 mmol), Pd(amphos)Cl₂ (8.66 mg, 0.012 mmol) and Na₂CO₃ (78 mg, 0.734 mmol) in DME (1.2 mL) and water (0.4 mL) was heated to 90° C. for 90 mins under N₂. The cooled reaction mixture was filtered, the solid washed with DME and then water and dried in vacuo to give the title compound, 82 mg, as a tan-colored solid. LCMS m/z=329 [M+H]⁺

### Preparation 307

### 1-(benzyloxy)-6-chloro-4-iodo-2,7-naphthyridine

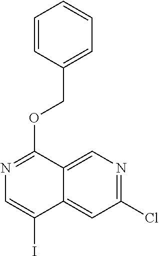 [SMILES: Clc1cc2c(I)cnc(OCc3ccccc3)c2cn1]

NaH (891 mg, 22.28 mmol) was added to a solution of benzyl alcohol (2.42 mL, 23.29 mmol) in DMF (60 mL) cooled in an ice bath and the solution stirred for 30 mins. 1,6-Dichloro-4-iodo-2,7-naphthyridine (Preparation 146, 6.58 g, 20.25 mmol) was added and the reaction allowed to warm slowly to rt overnight. The reaction mixture was poured into water and extracted with EtOAc (3×). The combined organic extracts were washed with water, then brine and dried over Na₂SO₄ and filtered. The filtrate was concentrated in vacuo and the crude product purified by Isco chromatography (0 to 20% EtOAc/Hex) to give the title compound, 3.61 g, 44.9% as a white solid. LCMS m/z=397 [M+H]⁺

### Preparation 308

### 1-(benzyloxy)-6-chloro-4-(4,4,5,5-tetramethyl-1,3,2-dioxaborolan-2-yl)-2,7-naphthyridine

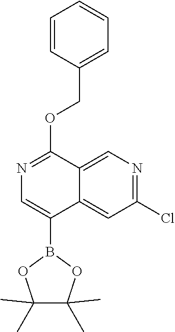 [SMILES: CC1(C)OB(c2cnc(OCc3ccccc3)c3cnc(Cl)cc23)OC1(C)C]

A mixture of 1-(benzyloxy)-6-chloro-4-iodo-2,7-naphthyridine (Preparation 307, 3 g, 7.56 mmol), (BPin)₂ (2.113 g, 8.32 mmol), KOAc (2.227 g, 22.69 mmol) and Pd(dppf)Cl₂ (277 mg, 0.378 mmol) in dioxane (25 mL) was stirred at 100° C. overnight under N₂. The cooled mixture was diluted with EtOAc, filtered through Celite® and the filtrate evaporated to give the crude product. This was purified by Isco chromatography (0 to 15% EtOAc/Hex) to give the title compound, 1.22 g, 40.6% as white solid. LCMS m/z=397 [M+H]⁺

### Preparation 309

### 1-(benzyloxy)-6-chloro-2,7-naphthyridin-4-ol

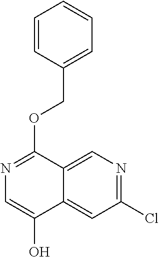 [SMILES: Oc1cnc(OCc2ccccc2)c2cnc(Cl)cc12]

Oxone as a solution in water (15 mL) was added drop wise to a suspension of 1-(benzyloxy)-6-chloro-4-(4,4,5,5-tetramethyl-1,3,2-dioxaborolan-2-yl)-2,7-naphthyridine (Preparation 308, 1.22 g, 3.08 mmol) in acetone (15 mL) in an ice bath and after complete addition, the reaction was allowed to warm to rt. The reaction was stirred for 2 h, then re-cooled in an ice bath. Saturated sodium bisulfite solution and water (20 mL) were added, the mixture filtered, the solid washed with water and dried in vacuo to give the title compound, 869 mg, 98% as an off-white solid. LCMS m/z=287 [M+H]⁺

### Preparation 310

### 1-(benzyloxy)-6-chloro-4-ethoxy-2,7-naphthyridine

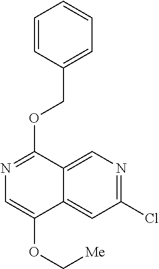 [SMILES: CCOc1cnc(OCc2ccccc2)c2cnc(Cl)cc12]

A mixture of 1-(benzyloxy)-6-chloro-2,7-naphthyridin-4-ol (Preparation 309, 40 mg, 0.14 mmol), ethyl iodide (26 mg, 0.167 mmol) and K₂CO₃ in DMF (0.5 mL) was stirred at rt for 1 h. The mixture was diluted with water, stirred at rt for 30 mins, the resulting solid filtered off and dried in vacuo to give the title compound, 36 mg, 81.6%, as a yellow solid. LCMS m/z=315 [M+H]⁺

### Preparation 311

### 6-chloro-4-ethoxy-2,7-naphthyridin-1(2H)-one

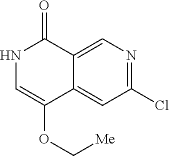 [SMILES: CCOc1c[nH]c(=O)c2cnc(Cl)cc12]

A solution of 1-(benzyloxy)-6-chloro-4-ethoxy-2,7-naphthyridine (Preparation 310, 247 mg, 0.785 mmol) in DCM (1 mL) and TFA (0.605 mL) was stirred at rt for 90 mins. The reaction was evaporated under reduced pressure and azeotroped with DCM and dried in vacuo to give the title compound, 161 mg, 91.1% as a yellow solid. LCMS m/z=225 [M+H]⁺

### Preparation 312

### 1,6-dichloro-4-ethoxy-2,7-naphthyridine

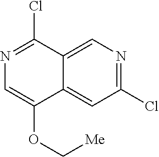 [SMILES: CCOc1cnc(Cl)c2cnc(Cl)cc12]

A mixture of 6-chloro-4-ethoxy-2,7-naphthyridin-1(2H)-one (Preparation 311, 266 mg, 0.785 mmol) in POCl₃ (0.732 mL) was heated to 90° C. for 1 h. The cooled mixture was evaporated under reduced pressure and azeotroped with toluene. The residue was dissolved in DCM and neutralised using aq. NaHCO₃ and the layers separated. The organic layer was dried over Na₂SO₄, filtered and evaporated under reduced pressure to give the title compound, 181 mg, 94.1% as pale yellow solid. LCMS m/z=245 [M+H]⁺

### Preparation 313

### 6-chloro-4-ethoxy-1-(2-methylazetidin-1-yl)-2,7-naphthyridine

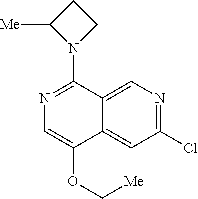 [SMILES: CCOc1cnc(N2CCC2C)c2cnc(Cl)cc12]

A mixture of 1,6-dichloro-4-ethoxy-2,7-naphthyridine (Preparation 312, 193 mg, 0.794 mmol), 2-methylazetidine hydrochloride (102 mg, 0.953 mmol) and K₂CO₃ (307 mg, 2.223 mmol) in DMF (2 mL) was heated at 100° C. for 6 h. The cooled mixture was diluted with water, extracted with EtOAc and the organic phase was washed with water (×2), brine and dried over Na₂SO₄. The mixture was filtered and evaporated under reduced pressure. The crude product was purified by Isco chromatography (0 to 40% EtOAc/Hex) to give the title compound, 51 mg, 23.1%, as a yellow solid. LCMS m/z=278 [M+H]⁺

### Preparation 314

### 4-bromo-6-chloro-1-methoxy-2,7-naphthyridine

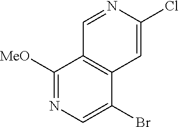 [SMILES: COc1ncc(Br)c2cc(Cl)ncc12]

Finely ground K₂CO₃ (155 mg, 1.124 mmol) was added to 4-bromo-1,6-dichloro-2,7-naphyridine (125 mg, 0.45 mmol) suspended in MeOH (4 mL) and the reaction stirred at rt for 1 h. The mixture was diluted with water, filtered, the solid washed with water and dried in vacuo to give the title compound, 101 mg, 81.9%, as an off white solid. LCMS m/z=274 [M+H]⁺

### Preparation 315

### 6-chloro-4-cyclopropyl-1-methoxy-2,7-naphthyridine

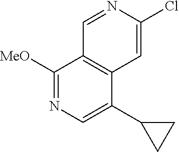 [SMILES: COc1ncc(C2CC2)c2cc(Cl)ncc12]

A mixture of 4-bromo-6-chloro-1-methoxy-2,7-naphthyridine (Preparation 314, 100 mg, 0.366 mmol), cyclopropylboronic acid (157 mg, 1.828 mmol), K₂CO₃ (152 mg, 1.10 mmol) and Pd(amphos)Cl₂ in dioxane (1.2 mL) and water (0.3 mL) was heated to 90° C. under N₂ for 1 h. The cooled mixture was diluted with EtOAc, washed with water and brine and dried over Na₂SO₄. The mixture was filtered, concentrated in vacuo and the residue purified by Isco chromatography (0 to 20% EtOAc/Hex) to give the title compound, 62 mg, 72.1%. LCMS m/z=235 [M+H]⁺

### Preparation 316

### 6-chloro-4-cyclopropyl-2,7-naphthyridin-1(2H)-one

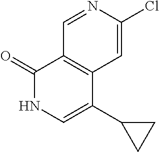 [SMILES: O=c1[nH]cc(C2CC2)c2cc(Cl)ncc12]

A solution of 6-chloro-4-cyclopropyl-1-methoxy-2,7-naphthyridine (Preparation 315, 31 mg, 0.132 mmol) and 1M HCl (0.5 mL) in THF (0.5 mL) was stirred overnight at rt. The reaction was stirred at 60° C. for a further 6 h and then at rt over the weekend. The reaction was concentrated in vacuo, water added and the resulting solid, filtered off and dried to give the title compound, 23 mg as a white solid. LCMS m/z=222 [M+H]⁺

### Preparation 317

### (2R,3S)-1-(6-chloro-4-cyclopropyl-2,7-naphthyridin-1-yl)-2-methylazetidin-3-ol

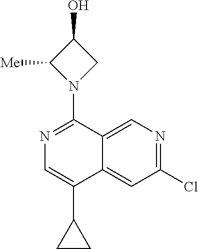 [SMILES: C[C@@H]1[C@@H](O)CN1c1ncc(C2CC2)c2cc(Cl)ncc12]

A mixture of 6-chloro-4-cyclopropyl-2,7-naphthyridin-1(2H)-one (Preparation 316, 23 mg, 0.104 mmol), (2R,3S)-2-methylazetidin-3-ol ((1S)-7,7-dimethyl-2-oxobicyclo[2.2.1]heptan-1-yl)methanesulfonate (Preparation 72, 40 mg, 0.125 mmol), HBTU (60 mg, 0.136 mmol) and DBU (47 mg, 0.313 mmol) in DMF (0.4 mL) was stirred at rt for 1 h. The mixture was diluted with water (5 mL), stirred for 1 h, filtered, washed with water and dried to give the title compound, 26 mg, 86.2% as a white solid. LCMS m/z=290 [M+H]⁺

### Preparation 318

### (R)-6-chloro-1-(2-methylazetidin-1-yl)-2,7-naphthyridin-4-ol

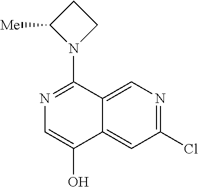 [SMILES: C[C@@H]1CCN1c1ncc(O)c2cc(Cl)ncc12]

Dioxane (3.5 mL) and water (3.5 mL) were added to (R)-4-bromo-6-chloro-1-(2-methylazetidin-1-yl)-2,7-naphthyridine (Preparation 152, 600 mg, 1.92 mmol), KOH (431 mg, 7.68 mmol), Pd₂(dba)₃ (35 mg, 0.038 mmol) and tetramethyl tBuXPhos (74 mg, 0.154 mmol) under N₂ and the reaction stirred at 90° C. for 45 mins. The cooled mixture was diluted with 10% MeOH/DCM (40 mL) and water and the pH adjusted to ˜7-8 with 1M HCl. The mixture was filtered through Celite®, washing through with water and the filtrate separated. The organic phase was dried over Na₂SO₄, filtered and concentrated in vacuo. The crude product was purified by Isco chromatography (0 to 10% MeOH/DCM) to give the title compound, 161 mg, 33.5%, as a yellow solid. LCMS m/z=250 [M+H]⁺

### Preparation 319

### (R)-6-chloro-4-isopropoxy-1-(2-methylazetidin-1-yl)-2,7-naphthyridine

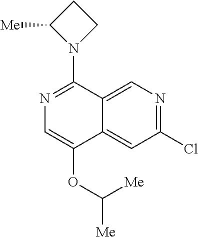 [SMILES: CC(C)Oc1cnc(N2CC[C@H]2C)c2cnc(Cl)cc12]

2-Iodopropane (68 mg, 0.4 mmol) and K₂CO₃ (17 mg, 0.12 mmol) were added to a solution of (R)-6-chloro-1-(2-methylazetidin-1-yl)-2,7-naphthyridin-4-ol (Preparation 318, 20 mg, 0.08 mmol) in DMF (0.4 mL) and the reaction stirred at rt for 2.5 h. The reaction was diluted with water, extracted with 5% MeOH/DCM, the combined organic extracts dried over Na₂SO₄, filtered and concentrated in vacuo. The residue was purified by Isco chromatography (0 to 40% EtOAc/Hex) to give the title compound, 9.5 mg, 40.7%, as yellow solid. LCMS m/z=292 [M+H]⁺

### Preparation 320

### (R)-6-chloro-4-(4,5-dihydrofuran-3-yl)-1-(2-methylazetidin-1-yl)-2,7-naphthyridine

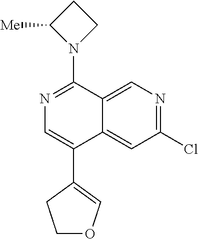 [SMILES: C[C@@H]1CCN1c1ncc(C2=COCC2)c2cc(Cl)ncc12]

The title compound was obtained as a pale yellow solid, 58 mg, 34.6% yield, from (R)-6-chloro-4-iodo-1-(2-methylazetidin-1-yl)-2,7-naphthyridine (Preparation 288) and 2-(4,5-dihydrofuran-3-yl)-4,4,5,5-tetramethyl-1,3,2-dioxaborolane, following a similar procedure to that described in Preparation 294. LCMS m/z=302 [M+H]⁺

### Preparation 321

### (R)-1-(6-chloro-1-(2-methylazetidin-1-yl)-2,7-naphthyridin-4-yl)ethan-1-one

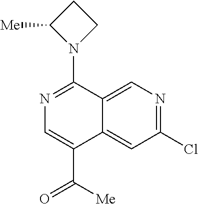 [SMILES: CC(=O)c1cnc(N2CC[C@H]2C)c2cnc(Cl)cc12]

A mixture of (R)-6-chloro-4-iodo-1-(2-methylazetidin-1-yl)-2,7-naphthyridine (Preparation 288, 730 mg, 2.03 mmol), tributyl(1-ethoxyvinyl)stannane (770 mg, 2.132 mmol) and Pd(PPh₃)₄ (117 mg, 0.102 mmol) in dioxane (6 mL) was heated at 1000 under N₂, overnight. The mixture was diluted with EtOAc, filtered through Celite® and the filtrate concentrated in vacuo. The product was dissolved in THF (5 mL), water (5 mL) and 1N HCl (1 mL) added and the mixture stirred vigorously overnight. The mixture was diluted with EtOAc, washed with brine and dried over Na₂SO₄, then filtered and evaporated under reduced pressure. The crude was purified by Isco chromatography (0 to 100% EtOAc/hexane) to give the title compound, 362 mg, 64.6%, as a yellow solid. LCMS m/z=276 [M+H]⁺

### Preparation 322

### 1-(6-chloro-1-((R)-2-methylazetidin-1-yl)-2,7-naphthyridin-4-yl)ethan-1-ol

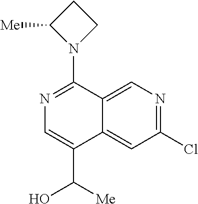 [SMILES: CC(O)c1cnc(N2CC[C@H]2C)c2cnc(Cl)cc12]

NaBH₄ (37 mg, 0.979 mmol) was added to an ice cooled solution of (R)-1-(6-chloro-1-(2-methylazetidin-1-yl)-2,7-naphthyridin-4-yl)ethan-1-one (Preparation 321, 180 mg, 0.653 mmol) in MeOH (4 mL) and the reaction allowed to warm slowly to rt overnight. The reaction was concentrated in vacuo, the residue partitioned between 5% MeOH/DCM and water with some ammonium chloride solution added. The organic layer was dried over Na₂SO₄, filtered and evaporated under reduced pressure to give the title compound, 173 mg as pale yellow foam. LCMS m/z=278 [M+H]⁺

### Preparation 323

### 6-chloro-1-((R)-2-methylazetidin-1-yl)-4-(1-(oxetan-3-ylmethoxy)ethyl)-2,7-naphthyridine

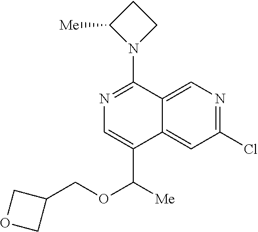 [SMILES: CC(OCC1COC1)c1cnc(N2CC[C@H]2C)c2cnc(Cl)cc12]

3-(Bromomethyl)oxetane (33 mg, 0.216 mmol), followed by NaH (6.91 mg, 0.173 mmol) were added to a solution of 1-(6-chloro-1-((R)-2-methylazetidin-1-yl)-2,7-naphthyridin-4-yl)ethan-1-ol (Preparation 322, 40 mg, 0.144 mmol) in DMF (0.75 mL) and the reaction stirred at rt for 3 h. Further 3-(bromomethyl)oxetane (99 mg in total) and NaH (80 mg in total) were added to the reaction over 4 h, and the reaction stirred overnight. The mixture was diluted with water, extracted with EtOAc, the organic layer washed with water and brine and dried over Na₂SO₄. The mixture was filtered and evaporated under reduced pressure. The crude was purified by Isco chromatography (10 to 100% EtOAc/Hex) to give the title compound, 33 mg, 65.9%, as off-white solid. LCMS m/z=348 [M+H]⁺

### Preparation 324

### methyl (R)-2-(6-chloro-1-(2-methylazetidin-1-yl)-2,7-naphthyridin-4-yl)acrylate

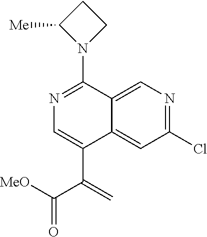 [SMILES: C=C(C(=O)OC)c1cnc(N2CC[C@H]2C)c2cnc(Cl)cc12]

A solution of (R)-6-chloro-4-iodo-1-(2-methylazetidin-1-yl)-2,7-naphthyridine (Preparation 288, 4.1 g, 11.4 mmol), methyl 2-(tributylstannyl)prop-2-enoate (5.10 g, 13.6 mmol), CuI (1.08 g, 5.7 mmol) and Pd(PPh₃)₄ (1.31 g, 1.14 mmol) in DMF (30 mL) was warmed to 80° C. for 2 h under N₂. The reaction mixture was diluted with water (100 mL), extracted with EtOAc (200 mL×3) and the combined organic extracts washed with brine (50 mL). The organic layer was dried over Na₂SO₄, filtered, evaporated under reduced pressure, and the crude purified by column chromatography (PE:EtOAc=1:1) to afford the title compound, 2.3 g, 63.5% as a yellow solid. LCMS m/z=318 [M+H]⁺

### Preparation 325

### methyl 2-(6-chloro-1-((R)-2-methylazetidin-1-yl)-2,7-naphthyridin-4-yl)propanoate

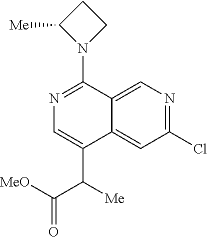 [SMILES: COC(=O)C(C)c1cnc(N2CC[C@H]2C)c2cnc(Cl)cc12]

The title compound was obtained as a yellow solid, 1.83 g, 75.9% yield, from methyl (R)-2-(6-chloro-1-(2-methylazetidin-1-yl)-2,7-naphthyridin-4-yl)acrylate (Preparation 324), following a similar procedure to that described in Preparation 148. LCMS m/z=320 [M+H]⁺

### Preparation 326

### 3-(6-chloro-1-((R)-2-methylazetidin-1-yl)-2,7-naphthyridin-4-yl)-2-methylbutan-2-ol

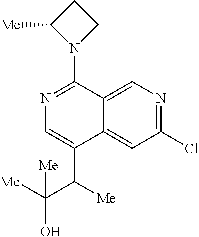 [SMILES: CC(c1cnc(N2CC[C@H]2C)c2cnc(Cl)cc12)C(C)(C)O]

MeMgBr solution (0.391 mL, 3M in ether) was added dropwise to a solution of methyl 2-(6-chloro-1-((R)-2-methylazetidin-1-yl)-2,7-naphthyridin-4-yl)propanoate (Preparation 325, 150 mg, 0.469 mmol) in THF (3 mL) at 0° C. under N₂, and the reaction allowed to warm to rt. Further MeMgBr (0.391 mL, 3M in ether) was added and the reaction stirred at rt overnight. The reaction was quenched with NH₄Cl solution and extracted with EtOAc. The organic layer was washed with brine, dried over Na₂SO₄, filtered and concentrated in vacuo. The crude was purified by Isco chromatography (0 to 80% EtOAc/Hex) to give the title compound as a viscous yellow oil, 91 mg, 60.6%. LCMS m/z=320 [M+H]⁺

### Preparation 327

### 2-(6-chloro-1-((R)-2-methylazetidin-1-yl)-2,7-naphthyridin-4-yl)-N,N-dimethylpropanamide

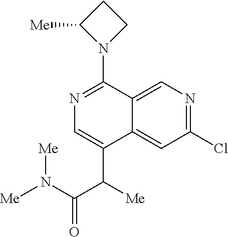 [SMILES: CC(C(=O)N(C)C)c1cnc(N2CC[C@H]2C)c2cnc(Cl)cc12]

A mixture of methyl 2-(6-chloro-1-((R)-2-methylazetidin-1-yl)-2,7-naphthyridin-4-yl)propanoate (Preparation 325, 63 mg, 0.206 mmol), HATU (102 mg, 0.268 mmol), dimethylamine (0.155 mL, 0.309 mmol) and DIPEA (0.09 mL, 0.515 mmol) in THF (1 mL) was stirred at rt overnight. The reaction was diluted with 5% MeOH/DCM, washed with water, dried over Na₂SO₄, filtered and evaporated to give the crude product. This was purified by Isco chromatography (0 to 10% MeOH/DCM) to give the title compound, 67 mg, 98%, as a pale yellow semi-solid. LCMS m/z=333 [M+H]⁺

### Preparation 328

### 2-(6-chloro-1-((R)-2-methylazetidin-1-yl)-2,7-naphthyridin-4-yl)propyl 4-methylbenzenesulfonate

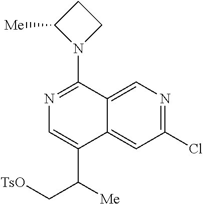 [SMILES: Cc1ccc(S(=O)(=O)OCC(C)c2cnc(N3CC[C@H]3C)c3cnc(Cl)cc23)cc1]

Tosyl chloride (137 mg, 0.72 mmol) was added to an ice cooled solution of 2-(6-chloro-1-((R)-2-methylazetidin-1-yl)-2,7-naphthyridin-4-yl)propan-1-ol (Preparation 298, 210 mg, 0.72 mmol) and DIPEA (112 mg, 0.864 mmol) in DCM (4 mL) and the mixture stirred for 20 mins. The ice bath was removed and the reaction allowed to warm to rt and stirred for 2 h. DMAP (25 mg, 0.205 mmol) was added and the reaction stirred overnight. The mixture was diluted with DCM, washed with ammonium chloride solution and dried over Na₂SO₄. The mixture was filtered, the filtrate concentrated in vacuo and the residue purified by Isco chromatography (0 to 50% EtOAc/Hex) to give the title compound, 155 mg, 48.3%, as pale yellow foam. LCMS m/z=446 [M+H]⁺

### Preparation 329

### 3-(6-chloro-1-((R)-2-methylazetidin-1-yl)-2,7-naphthyridin-4-yl)butanenitrile

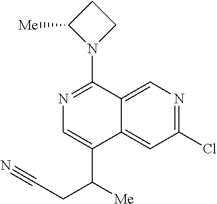 [SMILES: CC(CC#N)c1cnc(N2CC[C@H]2C)c2cnc(Cl)cc12]

NaCN (15 mg, 0.308 mmol) was added to a solution of 2-(6-chloro-1-((R)-2-methylazetidin-1-yl)-2,7-naphthyridin-4-yl)propyl 4-methylbenzenesulfonate (Preparation 328, 125 mg, 0.28 mmol) in DMSO (1 mL) and the reaction stirred at rt overnight. The mixture was diluted with water, extracted with EtOAc, the combined organic layers washed with water, brine and dried over Na₂SO₄. The mixture was filtered, concentrated in vacuo and the residue purified by Isco chromatography (0 to 60% EtOAc/Hex) to give the title compound, 70 mg, 83% as yellow semi-solid. LCMS m/z=301 [M+H]⁺

### Preparations 330 and 331

### tert-butyl (S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate and tert-butyl (R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate

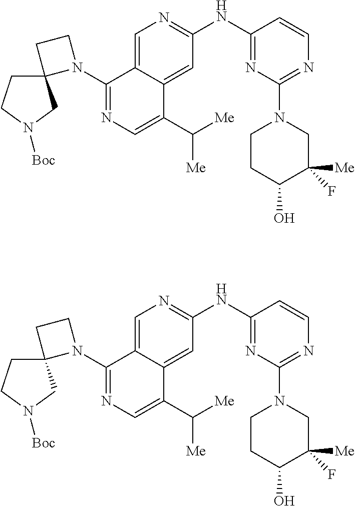 [SMILES: CC(C)c1cnc(N2CC[C@@]23CCN(C(=O)OC(C)(C)C)C3)c2cnc(Nc3ccnc(N4CC[C@@H](O)[C@@](C)(F)C4)n3)cc12 ; CC(C)c1cnc(N2CC[C@]23CCN(C(=O)OC(C)(C)C)C3)c2cnc(Nc3ccnc(N4CC[C@@H](O)[C@@](C)(F)C4)n3)cc12]

To a solution of tert-butyl 1-(6-chloro-4-isopropyl-2,7-naphthyridin-1-yl)-1,6-diazaspiro[3.4]octane-6-carboxylate (Preparation 187, 180 mg, 0.432 mmol) in dioxane was added (3S,4R)-1-(4-aminopyrimidin-2-yl)-3-fluoro-3-methylpiperidin-4-ol (Preparation 8, 107 mg, 0.475 mmol), Cs₂CO₃ (420 mg, 1.29 mmol) and Brettphos Pd G3 (39.1 mg, 0.043 mmol) under N₂ and the mixture was stirred at 100° C. for 2 h. The cooled reaction mixture was diluted with water (20 mL), extracted with EtOAc (2×20 mL) and the organic layers combined. The resulting solution was washed with brine (20 mL), dried over anhydrous Na₂SO₄ and concentrated in vacuo. The crude product was purified by prep-TLC with DCM:MeOH=20:1 to give tert-butyl 1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate, 200 mg (76.6%) as yellow solid. LCMS m/z=607 [M+H]⁺

This was further purified by chiral HPLC using a CHIRALPAK IA-3 4.6×50 mm 3 μm column, Mobile phase:Hex (0.1% DEA):EtOH=50:50 at 1 mL/min to provide:

Peak 1: tert-butyl (S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate or tert-butyl (R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate, 80 mg.

Peak 2: tert-butyl (R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate or tert-butyl (S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate, 80 mg.

### Preparation 332

### 5-bromo-N-tert-butyl-2-chloroisonicotinamide

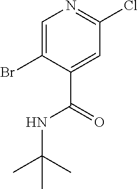

A solution of 5-bromo-2-chloropyridine-4-carboxylic acid (4 g, 16.9 mmol), 2-methylpropan-2-amine (1.47 g, 20.2 mmol), EDC HCl (4.85 g, 25.3 mmol) and HOBT (3.41 g, 25.3 mmol) in DMF (30 mL), under N₂ was stirred overnight at rt. The reaction was diluted with water, extracted with EtOAc, the organic layers were combined, dried over anhydrous Na₂SO₄ and concentrated in vacuo. The residue was purified by column chromatography on silica gel (PE/EtOAc, 2:1) to give the title compound, 3 g (60.9%) as a white solid. LCMS m/z=293 [M+H]*; ¹H NMR (300 MHz, DMSO-d₆) δ: 8.64 (s, 1H), 8.30 (s, 1H), 7.58 (s, 1H), 1.36 (s, 9H).

### Preparation 333

### (E)-N-(tert-butyl)-2-chloro-5-(2-ethoxyvinyl)isonicotinamide

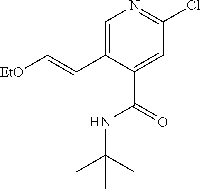 [SMILES: CCO/C=C/c1cnc(Cl)cc1C(=O)NC(C)(C)C]

A solution of 5-bromo-N-tert-butyl-2-chloropyridine-4-carboxamide (Preparation 332, 2 g, 6.85 mmol), 2-[(E)-2-ethoxyethenyl]-4,4,5,5-tetramethyl-1,3,2-dioxaborolane (1.49 g, 7.53 mmol), Cs₂CO₃ (4.46 g, 13.7 mmol) and Pd(dppf)Cl₂ (501 mg, 0.685 mmol) in dioxane (30 mL) and H₂O (6 mL) was stirred for 2 h at 80° C. The cooled solution was diluted with water and extracted with EtOAc, the combined organic layers dried over anhydrous Na₂SO₄ and concentrated in vacuo. The residue was purified by silica gel chromatography eluting with PE/EtOAc (2:1) to give the title compound, 1.2 g (62.1%) as a yellow solid. LCMS m/z=283 [M+H]⁺; ¹H NMR (300 MHz, DMSO-d₆) δ: 8.55 (s, 1H), 8.20 (s, 1H), 7.35 (d, 1H), 7.28 (s, 1H), 5.79 (d, 1H), 3.90 (q, 2H), 1.35 (s, 9H), 1.26 (t, 3H).

### Preparation 334

### 7-chloro-2,6-naphthyridin-1(2H)-one

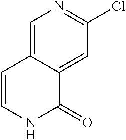 [SMILES: O=c1[nH]ccc2cnc(Cl)cc12]

A solution of (E)-N-(tert-butyl)-2-chloro-5-(2-ethoxyvinyl)isonicotinamide (Preparation 333, 1.2 g, 4.24 mmol) in TFA (20 mL) was stirred overnight at 100° C. The resulting mixture was cooled and evaporated under reduced pressure to give the title compound, 600 mg, as a red solid. The crude product was used directly without any further purification. LCMS m/z=181 [M+H]⁺.

### Preparation 335

### 4-bromo-7-chloro-2,6-naphthyridin-1(2H)-one

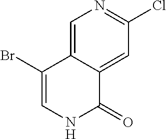 [SMILES: O=c1[nH]cc(Br)c2cnc(Cl)cc12]

A solution of 7-chloro-2,6-naphthyridin-1(2H)-one (Preparation 334, 3 g, 16.6 mmol) and NBS (3.54 g, 19.9 mmol) in DCM (40 mL) was stirred for 1 h at rt. The resulting solid was collected by filtration to give the title compound, 3 g (69.7%) as a white solid. LCMS m/z=261 [M+H]⁺

### Preparation 336

### 4-bromo-7-chloro-2,6-naphthyridin-1-yl trifluoromethanesulfonate

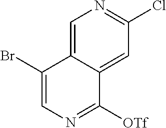 [SMILES: O=S(=O)(Oc1ncc(Br)c2cnc(Cl)cc12)C(F)(F)F]

A solution of 4-bromo-7-chloro-2,6-naphthyridin-1(2H)-one (Preparation 335, 1 g, 3.85 mmol) and TEA (777 mg, 7.70 mmol) in DCM (15 mL) was cooled to −78° C., and then Tf₂O (4.34 g, 15.4 mmol) was added drop wise over 10 min. The reaction was stirred for 0.5 h at −78° C., then warmed to rt and stirred for 0.5 h. The reaction was quenched with ice-water (2 mL), extracted with DCM, the organic layers combined, dried over anhydrous Na₂SO₄ and concentrated in vacuo. The residue was purified by silica gel column eluting with EtOAc:PE (0-10%) to give the title compound, 1 g (66.6%) as a white solid. LCMS m/z=393 [M+H]⁺

### Preparation 337

### 4-bromo-7-chloro-1-iodo-2,6-naphthyridine

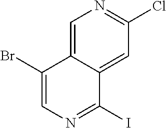 [SMILES: Clc1cc2c(I)ncc(Br)c2cn1]

A mixture of 4-bromo-7-chloro-2,6-naphthyridin-1-yl trifluoromethanesulfonate (Preparation 336, 500 mg, 1.27 mmol) and NaI (952 mg, 6.35 mmol) in MeCN (9 mL) was cooled to 0° C. and a solution of trifluoromethanesulfonate acid (381 mg, 2.54 mmol) in MeCN (1 mL) was added drop wise over 10 min. The reaction was then stirred at rt for 1.5 h. The reaction mixture was extracted with EtOAc, the organic layers combined, washed with brine, dried over anhydrous Na₂SO₄ and evaporated under reduced pressure to give the title compound, 500 mg as a dark solid. LCMS m/z=369 [M+H]⁺.

### Preparation 338

### 4-bromo-7-chloro-1-(prop-1-en-2-yl)-2,6-naphthyridine

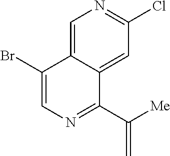

The title compound was obtained as a light yellow oil, 200 mg, 52.3% yield, from 4-bromo-7-chloro-1-iodo-2,6-naphthyridine (Preparation 337) and 4,4,5,5-tetramethyl-2-(prop-1-en-2-yl)-1,3,2-dioxaborolane, following the procedure described in Preparation 154. LCMS m/z=285 [M+H]⁺.

### Preparation 339

### 2-(4-bromo-7-chloro-2,6-naphthyridin-1-yl)prop-2-en-1-ol

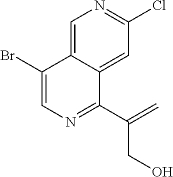 [SMILES: C=C(CO)c1ncc(Br)c2cnc(Cl)cc12]

The title compound was obtained as a light yellow solid, 800 mg, from 4-bromo-7-chloro-1-iodo-2,6-naphthyridine (Preparation 337), and 2-(4,4,5,5-tetramethyl-1,3,2-dioxaborolan-2-yl)prop-2-en-1-ol following a similar procedure to that described in Preparation 111. LCMS m/z=299 [M+H]⁺

### Preparation 340

### methyl 2-(4-bromo-7-chloro-2,6-naphthyridin-1-yl)acrylate

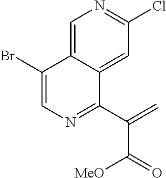 [SMILES: C=C(C(=O)OC)c1ncc(Br)c2cnc(Cl)cc12]

The title compound was obtained as a yellow solid, 1.1 g, 62.1% yield, from 4-bromo-7-chloro-1-iodo-2,6-naphthyridine (Preparation 337) and methyl 2-(tributylstannyl)prop-2-enoate, following the procedure described in Preparation 324. LCMS m/z=327 [M+H]⁺

### Preparation 341

### 4-bromo-7-chloro-1-isopropyl-2,6-naphthyridine

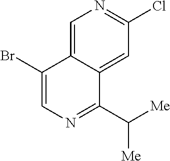 [SMILES: CC(C)c1ncc(Br)c2cnc(Cl)cc12]

The title compound was obtained as a yellow solid, 100 mg, 62.1% yield, from 4-bromo-7-chloro-1-(prop-1-en-2-yl)-2,6-naphthyridine (Preparation 338) following the procedure described in Preparation 253. LCMS m/z=287 [M+H].

### Preparation 342

### 2-(4-bromo-7-chloro-2,6-naphthyridin-1-yl)propan-1-ol

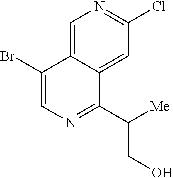 [SMILES: CC(CO)c1ncc(Br)c2cnc(Cl)cc12]

The title compound was obtained as a light yellow solid, from 2-(4-bromo-7-chloro-2,6-naphthyridin-1-yl)prop-2-en-1-ol (Preparation 339), following a similar procedure to that described in Preparation 299. LCMS m/z=301 [M+H]⁺

### Preparation 343

### methyl 2-(4-bromo-7-chloro-2,6-naphthyridin-1-yl)propanoate

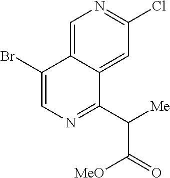 [SMILES: COC(=O)C(C)c1ncc(Br)c2cnc(Cl)cc12]

The title compound was obtained as a yellow solid, from methyl 2-(4-bromo-7-chloro-2,6-naphthyridin-1-yl)acrylate (Preparation 349), following the procedure described in Preparation 148. LCMS m/z=329 [M+H]⁺

### Preparation 344

### 7-chloro-4-(3-fluoroazetidin-1-yl)-1-isopropyl-2,6-naphthyridine

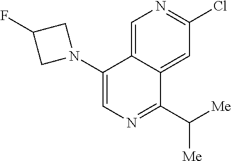 [SMILES: CC(C)c1ncc(N2CC(F)C2)c2cnc(Cl)cc12]

A mixture of 4-bromo-7-chloro-1-isopropyl-2,6-naphthyridine (Preparation 341, 114.2 mg, 0.4 mmol), 3-fluoroazetidine hydrochloride (89.2 mg, 0.8 mmol), [Pd(allyl)(Brett)]OTf (33.9 mg, 0.04 mmol) and Cs₂CO₃ (260.8 mg, 0.8 mmol) in dioxane (4 mL) was stirred at 100° C. for 4 h. The cooled reaction mixture was concentrated in vacuo and the residue purified by silica gel column (MeOH-DCM (1:100) to give the title compound, 95 mg, 84.8%. LCMS m/z=280 [M+H]⁺

### Preparation 345

### 1-(7-chloro-1-isopropyl-2,6-naphthyridin-4-yl)-N,N-dimethylazetidine-3-carboxamide

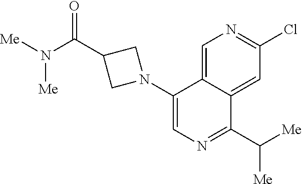 [SMILES: CC(C)c1ncc(N2CC(C(=O)N(C)C)C2)c2cnc(Cl)cc12]

To a solution of 4-bromo-7-chloro-1-isopropyl-2,6-naphthyridine (Preparation 341, 200 mg, 0.70 mmol) in dioxane was added N,N-dimethylazetidine-3-carboxamide (107 mg, 0.84 mmol), Cs₂CO₃ (684 mg, 2.1 mmol) and XantphosPd G2 (62.1 mg, 0.07 mmol) and the reaction mixture was stirred at 100° C. for 2 h under N₂. The reaction mixture was cooled to rt, diluted with water (20 mL), extracted with EtOAc (2×20 mL) and the organic layers combined. The solution was washed with brine (20 mL), dried over anhydrous Na₂SO₄ and concentrated in vacuo. The crude product was purified by prep-TLC to give the title compound, 200 mg (85.8%) as a yellow solid. LCMS m/z=333 [M+H]⁺

### Preparation 346

### (R)-7-chloro-1-isopropyl-4-(2-methylazetidin-1-yl)-2,6-naphthyridine

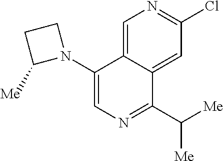 [SMILES: CC(C)c1ncc(N2CC[C@H]2C)c2cnc(Cl)cc12]

The title compound was obtained as a yellow oil from (2R)-2-methylazetidine and 4-bromo-7-chloro-1-isopropyl-2,6-naphthyridine (Preparation 341), following a similar procedure to that described in Preparation 345. LCMS m/z=276 [M+H]⁺

### Preparation 347

### tert-butyl 1-(7-chloro-1-isopropyl-2,6-naphthyridin-4-yl)-1,6-diazaspiro[3.3]heptane-6-carboxylate

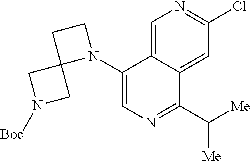 [SMILES: CC(C)c1ncc(N2CCC23CN(C(=O)OC(C)(C)C)C3)c2cnc(Cl)cc12]

A solution of 4-bromo-7-chloro-1-isopropyl-2,6-naphthyridine (Preparation 341, 300 mg, 1.05 mmol), tert-butyl 1,6-diazaspiro[3.3]heptane-6-carboxylate (208 mg, 1.05 mmol), Pd₂(dba)₃ (54 mg, 0.0525 mmol), Xantphos (60.6 mg, 0.105 mmol) and Cs₂CO₃ (684 mg, 2.10 mmol) in toluene (10 mL) was heated at 110° C. for 2 h under N₂. The cooled reaction was filtered and the filtrate was concentrated in vacuo. The crude product was purified by prep TLC (PE:EtOAc=1:1) to give the title compound, 110 mg, 26%, as a yellow solid. LCMS m/z=403 [M+H]⁺

### Preparation 348

### (2R,3S)-1-(7-chloro-1-isopropyl-2,6-naphthyridin-4-yl)-2-methylazetidin-3-ol

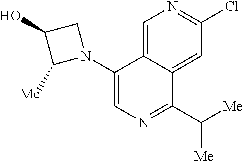 [SMILES: CC(C)c1ncc(N2C[C@H](O)[C@H]2C)c2cnc(Cl)cc12]

A mixture of (2R,3S)-2-methylazetidin-3-ol trifluoroacetate (Preparation 73, 110 mg, 1.263 mmol), 4-bromo-7-chloro-1-isopropyl-2,6-naphthyridine (Preparation 341, 360.6 mg, 1.263 mmol), Cs₂CO₃ (1.238 g, 3.79 mmol) and BINAP Pd G3 (115.8 mg, 0.126 mmol) in dioxane (2 mL), was stirred for 3 h at 90° C. under N₂ in a sealed vessel. The reaction was quenched by the addition of aq. Na₂S₂O₃ (1 mL) and the resulting solids filtered off. The filtrate was extracted with EtOAc (3×2 mL) and concentrated in vacuo. The residue was purified by silica gel column eluting with EtOAc:PE (3:1) to give the title compound, 70 mg (19.0%) as a light yellow solid. LCMS m/z=292 [M+H]⁺

### Preparation 349

### 2-(7-chloro-4-((R)-2-methylazetidin-1-yl)-2,6-naphthyridin-1-yl)propan-1-ol

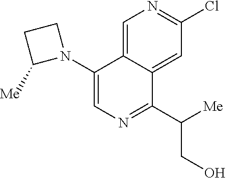 [SMILES: CC(CO)c1ncc(N2CC[C@H]2C)c2cnc(Cl)cc12]

The title compound was obtained as a yellow solid, 260 mg, 59.8% yield, from 2-(4-bromo-7-chloro-2,6-naphthyridin-1-yl)propan-1-ol (Preparation 342) and (2R)-2-methylazetidine, following a similar procedure to that described in Preparation 123. LCMS m/z=292 [M+H]⁺

### Preparation 350

### methyl 2-(7-chloro-4-((R)-2-methylazetidin-1-yl)-2,6-naphthyridin-1-yl)propanoate

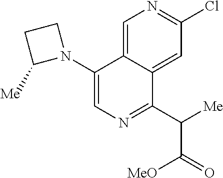 [SMILES: COC(=O)C(C)c1ncc(N2CC[C@H]2C)c2cnc(Cl)cc12]

The title compound was obtained as a light yellow solid, 450 mg, 51.5%, from methyl 2-(4-bromo-7-chloro-2,6-naphthyridin-1-yl)propanoate (Preparation 343) and (2R)-2-methylazetidine, following a similar procedure to that described in Preparation 120.

### Preparation 351

### 2-(7-chloro-4-((R)-2-methylazetidin-1-yl)-2,6-naphthyridin-1-yl)propanamide

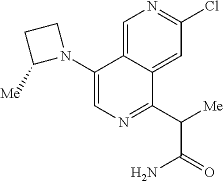 [SMILES: CC(C(N)=O)c1ncc(N2CC[C@H]2C)c2cnc(Cl)cc12]

A mixture of methyl 2-(7-chloro-4-((R)-2-methylazetidin-1-yl)-2,6-naphthyridin-1-yl)propanoate (Preparation 350, 150 mg, 0.469 mmol) in methanolic ammonia (5 mL) was stirred at 100° C. for 2 days in a sealed tube. The cooled solution was concentrated in vacuo and the residue was purified by prep-TLC with DCM/MeOH (20:1) to give the title compound, 135 mg, 94.4%, as a light yellow solid. LCMS m/z=305 [M+H]⁺

### Preparation 352

### 2-(7-chloro-4-((R)-2-methylazetidin-1-yl)-2,6-naphthyridin-1-yl)propanenitrile

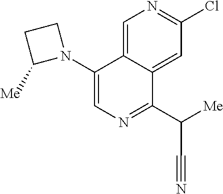 [SMILES: CC(C#N)c1ncc(N2CC[C@H]2C)c2cnc(Cl)cc12]

A solution of 2-(7-chloro-4-((R)-2-methylazetidin-1-yl)-2,6-naphthyridin-1-yl)propanamide (Preparation 351, 135 mg, 0.442 mmol), TEA (89.4 mg, 0.884 mmol) and trifluoroacetic anhydride (369 mg, 1.76 mmol) in DCM (1.5 mL) was stirred at rt for 2 h. The resulting solution was concentrated in vacuo and the residue was purified by prep-TLC (DCM/MeOH, 30:1) to give the title compound, 110 mg, 86.7%, as a white solid. LCMS m/z=287 [M+H]⁺

### Preparation 353

### 7-chloro-1-isopropyl-4-(1-methyl-1H-pyrazol-3-yl)-2,6-naphthyridine

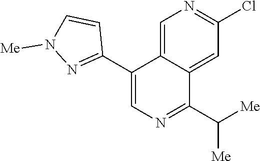 [SMILES: CC(C)c1ncc(-c2ccn(C)n2)c2cnc(Cl)cc12]

A solution of 4-bromo-7-chloro-1-isopropyl-2,6-naphthyridine (Preparation 341, 100 mg, 0.350 mmol), 1-methyl-3-(4,4,5,5-tetramethyl-1,3,2-dioxaborolan-2-yl)-1H-pyrazole (80.1 mg, 0.385 mmol), K₂CO₃ (96.6 mg, 0.70 mmol) and Pd(dppf)Cl₂ (12.8 mg, 0.0175 mmol) in dioxane (3 mL) and H₂O (0.6 mL), was stirred for 1 h at 80° C. The resulting solution was extracted with EtOAc, the combined organic layers, dried over anhydrous Na₂SO₄ and concentrated in vacuo. The residue was purified by prep-TLC with PE/EtOAc (2:1) to give the title compound, 40 mg, 39.9% as a yellow oil. LCMS m/z=287 [M+H]⁺

### Preparation 354

### 7-chloro-1-isopropyl-4-(1-methyl-1H-1,2,3-triazol-4-yl)-2,6-naphthyridine

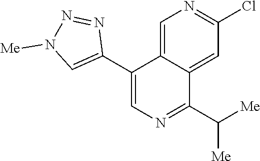 [SMILES: CC(C)c1ncc(-c2cn(C)nn2)c2cnc(Cl)cc12]

The title compound was obtained as a light yellow solid, from 4-bromo-7-chloro-1-isopropyl-2,6-naphthyridine (Preparation 341) and (1-methyl-1H-1,2,3-triazol-4-yl)boronic acid, following a similar procedure to that described in Preparation 353. LCMS m/z=288 [M+H]⁺

### Preparation 355

### 7-chloro-1-isopropyl-4-(1-methyl-1H-pyrazol-4-yl)-2,6-naphthyridine

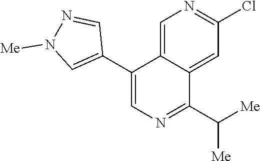 [SMILES: CC(C)c1ncc(-c2cnn(C)c2)c2cnc(Cl)cc12]

The title compound was obtained as a yellow oil, from 4-bromo-7-chloro-1-isopropyl-2,6-naphthyridine (Preparation 341) and 1-methyl-4-(4,4,5,5-tetramethyl-1,3,2-dioxaborolan-2-yl)-1H-pyrazole, following a similar procedure to that described in Preparation 353. LCMS m/z=287 [M+H]⁺

### Preparation 356

### (7-chloro-1-isopropyl-2,6-naphthyridin-4-yl)boronic acid

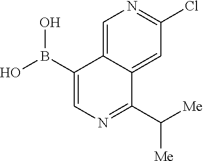 [SMILES: CC(C)c1ncc(B(O)O)c2cnc(Cl)cc12]

A solution of 4-bromo-7-chloro-1-isopropyl-2,6-naphthyridine (Preparation 341, 500 mg, 1.75 mmol) in dioxane (10 mL), (Bpin)₂ (665 mg, 2.62 mmol), KOAc (343 mg, 3.50 mmol) and Pd(dppf)Cl₂ (128 mg, 0.175 mmol) was stirred for 1 h at 90° C. under N₂. The cooled solution was extracted with EtOAc, the organic layers combined, dried over anhydrous Na₂SO₄ and evaporated under reduced pressure to give the title compound, 400 mg (91.3%) as a brown oil. LCMS m/z=251 [M+H]⁺

### Preparation 357

### 7-chloro-1-isopropyl-4-(1-methyl-1H-1,2,4-triazol-3-yl)-2,6-naphthyridine

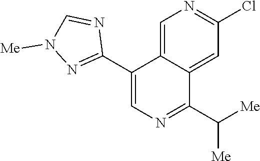 [SMILES: CC(C)c1ncc(-c2ncn(C)n2)c2cnc(Cl)cc12]

A solution of (7-chloro-1-isopropyl-2,6-naphthyridin-4-yl)boronic acid (Preparation 356, 100 mg, 0.399 mmol), 3-bromo-1-methyl-1H-1,2,4-triazole (64.6 mg, 0.399 mmol), K₂CO₃ (110 mg, 0.798 mmol) and Pd(dtbpf)Cl₂ (26.0 mg, 0.040 mmol) in dioxane (3 mL) and H₂O (0.6 mL), was stirred for 1 h at 80° C. under N₂. The cooled solution was extracted with EtOAc, the organic layers combined, dried over anhydrous Na₂SO₄ and concentrated in vacuo. The residue was purified by prep-TLC with PE/EtOAc (2:1) to give the title compound, 30 mg, 26.3%, as a yellow oil. LCMS m/z=288 [M+H]⁺

### Preparation 358

### 2-(7-chloro-1-isopropyl-2,6-naphthyridin-4-yl)-5-methyl-1,3,4-oxadiazole

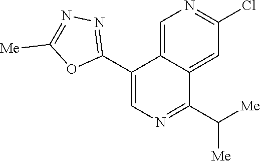 [SMILES: Cc1nnc(-c2cnc(C(C)C)c3cc(Cl)ncc23)o1]

The title compound was obtained as a yellow oil, 40 mg, 83% from (7-chloro-1-isopropyl-2,6-naphthyridin-4-yl)boronic acid (Preparation 356) and 2-bromo-5-methyl-1,3,4-oxadiazole, following a similar procedure to that described in Preparation 357, except Pd(dppf)Cl₂ was used as the catalyst. LCMS m/z=289 [M+H]⁺

### Preparation 359

### 7-chloro-1-isopropyl-2,6-naphthyridine-4-carbonitrile

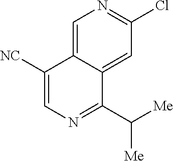 [SMILES: CC(C)c1ncc(C#N)c2cnc(Cl)cc12]

A mixture of 4-bromo-7-chloro-1-isopropyl-2,6-naphthyridine (Preparation 341, 200 mg, 0.70 mmol), Zn(CN)₂ (82.1 mg, 0.70 mmol) and Pd(PPh₃)₄ (87.0 mg, 0.07 mmol) in DMF (15 mL) was stirred at 90° C. for 16 h. The cooled mixture was concentrated in vacuo, the residue diluted with water and extracted with EtOAc. The organic layer was dried over Na₂SO₄, filtered and concentrated in vacuo. The residue was purified by prep-TLC (EtOAc:PE=1:10) to afford the title compound, 60 mg as a light yellow solid. LCMS m/z=232 [M+H]⁺

### Preparation 360

### 7-chloro-N-hydroxy-1-isopropyl-2,6-naphthyridine-4-carboximidamide

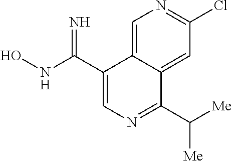 [SMILES: CC(C)c1ncc(C(=N)NO)c2cnc(Cl)cc12]

A mixture of 7-chloro-1-isopropyl-2,6-naphthyridine-4-carbonitrile (Preparation 359, 130 mg, 0.561 mmol) and hydroxylamine (300 mg, 4.54 mmol) in MeOH (20 mL) was stirred at 70° C. for 1 h. The mixture was concentrated in vacuo and the residue purified by prep-TLC (DCM:MeOH=20:1) to give the title compound, 70 mg, 47.1% as a light yellow solid. LCMS m/z=265 [M+H]⁺

### Preparation 361

### (Z)—N-((7-chloro-1-isopropyl-2,6-naphthyridin-4-yl)(hydroxyamino)methylene)acetamide

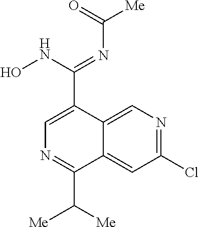 [SMILES: CC(=O)/N=C(\NO)c1cnc(C(C)C)c2cc(Cl)ncc12]

To a solution of 7-chloro-N-hydroxy-1-isopropyl-2,6-naphthyridine-4-carboximidamide (Preparation 360, 65 mg, 0.245 mmol) and TEA (123 mg, 1.22 mmol) in DCM (20 mL) was added acetyl chloride (57.6 mg, 0.735 mmol) and the reaction stirred at rt for 1 h. The reaction was quenched with water, extracted with EtOAc and the organic layer dried over Na₂SO₄. The mixture was filtered, and concentrated to dryness to afford the title compound, 75 mg. LCMS m/z=307 [M+H]⁺

### Preparation 362

### 3-(7-chloro-1-isopropyl-2,6-naphthyridin-4-yl)-5-methyl-1,2,4-oxadiazole

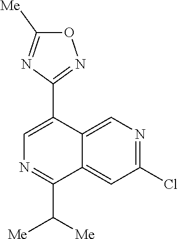 [SMILES: Cc1nc(-c2cnc(C(C)C)c3cc(Cl)ncc23)no1]

A solution of (Z)—N-((7-chloro-1-isopropyl-2,6-naphthyridin-4-yl)(hydroxyamino)methylene)acetamide (Preparation 361, 65 mg, 0.211 mmol) in toluene (20 mL) was stirred at 100° C. for 16 h. The cooled mixture was concentrated in vacuo and the residue was purified by prep-TLC (EtOAc:PE=1:4) to give the title compound, 50 mg as a white solid. LCMS m/z=289 [M+H]⁺

### Preparation 363

### 6-chloro-4-isobutyrylnicotinic acid

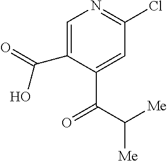 [SMILES: CC(C)C(=O)c1cc(Cl)ncc1C(=O)O]

To a stirred solution of n-BuLi (100 mL) in THF at −78° C., was added trimethyl phosphate (40.1 g, 285 mmol) dropwise and the mixture was allowed to warm to 0° C. and stirred for 1 h. The solution was recooled to −78° C., a solution of 6-chloropyridine-3-carboxylic acid (15 g, 95.2 mmol) in THF was added dropwise and the reaction was stirred for 1.5 h. N-Methoxy-N,2-dimethylpropanamide (37.3 g, 285 mmol) was added and the reaction mixture was allowed to warm to rt and stirred for 4 h. The mixture was quenched by aq. NH₄Cl at 0° C. and the pH adjusted to 5-6 with citric acid, then extracted with EtOAc. The combined organic layers were washed with brine, dried over Na₂SO₄, filtered and evaporated under reduced pressure to give the title compound, as a yellow oil. LCMS m/z=228 [M+H]⁺

### Preparation 364

### 7-chloro-1-isopropylpyrido[3,4-d]pyridazin-4(3H)-one

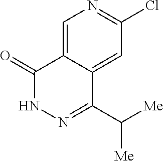 [SMILES: CC(C)c1n[nH]c(=O)c2cnc(Cl)cc12]

To a solution of 6-chloro-4-(2-methylpropanoyl)pyridine-3-carboxylic acid (Preparation 363, 11 g, 48.3 mmol) in IPA was added NH₂NH₂·H₂O (3.62 g, 72.4 mmol) and the reaction stirred at 70° C. for 3 h. The cooled mixture was filtered, the filtrate was concentrated in vacuo to 10 mL, and then filtered. The filtered solids were combined to give the title compound, as a yellow solid (6 g, crude). LCMS m/z=224 [M+H]⁺

### Preparation 365

### 4,7-dichloro-1-isopropylpyrido[3,4-d]pyridazine

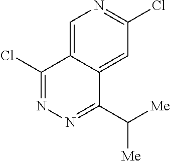 [SMILES: CC(C)c1nnc(Cl)c2cnc(Cl)cc12]

To POCl₃ was added 7-chloro-1-isopropylpyrido[3,4-d]pyridazin-4(3H)-one (Preparation 364, 100 mg, 0.447 mmol) and the reaction was stirred overnight at 100° C. The cooled mixture was evaporated under reduced pressure to afford the title compound, that was used without further purification. LCMS m/z=242 [M+H]⁺

### Preparation 366

### 7-chloro-4-(2-(difluoromethyl)azetidin-1-yl)-1-isopropylpyrido[3,4-d]pyridazine

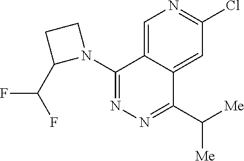 [SMILES: CC(C)c1nnc(N2CCC2C(F)F)c2cnc(Cl)cc12]

The title compound was obtained as a light yellow solid, 80 mg, 40%, from 4,7-dichloro-1-isopropylpyrido[3,4-d]pyridazine (Preparation 365) and 2-(difluoromethyl)azetidine following the procedure described in Preparation 170. LCMS m/z=313 [M+H]⁺

### Preparation 367

### 7-chloro-1-isopropyl-4-(2-methylazetidin-1-yl)pyrido[3,4-d]pyridazine

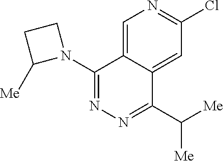 [SMILES: CC(C)c1nnc(N2CCC2C)c2cnc(Cl)cc12]

The title compound was obtained as a light yellow solid, 200 mg, 70% from 4,7-dichloro-1-isopropylpyrido[3,4-d]pyridazine (Preparation 365) and 2-methylazetidine, following the procedure described in Preparation 170. LCMS m/z=277 [M+H]⁺

### Preparation 368

### 7-chloro-1-isopropyl-4-(pyrrolidin-1-yl)pyrido[3,4-d]pyridazine

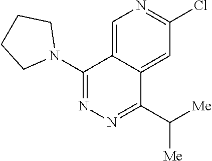 [SMILES: CC(C)c1nnc(N2CCCC2)c2cnc(Cl)cc12]

A mixture of pyrrolidine (26.4 mg, 0.371 mmol), 4,7-dichloro-1-isopropylpyrido[3,4-d]pyridazine (Preparation 365, 100 mg, 0.413 mmol) and TEA (83.4 mg, 0.827 mmol) in butan-2-ol (2 mL) was stirred at 80° C. for 12 h under N₂. The cooled reaction mixture was concentrated in vacuo and the residue purified by HPLC-30 to give the title compound, 48 mg, 42% yield, as a yellow solid. ¹H NMR (400 MHz, MeOD-d₄) δ: 9.51 (s, 1H), 8.02 (s, 1H), 3.95-3.92 (m, 4H), 3.72-3.62 (m, 1H), 2.15-2.09 (m, 4H), 1.42 (d, 6H)

### Preparation 369

### 2-(1-(benzyloxy)-6-chloro-2,7-naphthyridin-4-yl)prop-2-en-1-ol

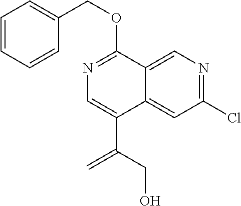 [SMILES: C=C(CO)c1cnc(OCc2ccccc2)c2cnc(Cl)cc12]

1-(Benzyloxy)-6-chloro-4-(4,4,5,5-tetramethyl-1,3,2-dioxaborolan-2-yl)-2,7-naphthyridine (Preparation 308, 680 mg, 1.714 mmol), 2-bromoprop-2-en-1-ol (258 mg, 1.886 mmol) and Na₂CO₃ (545 mg, 5.14 mmol) in DME (3 mL) and water (1 mL) were heated to 85° C. overnight under N₂. The cooled mixture was diluted with EtOAc and water and the mixture filtered through Celite®, and the filtrate separated. The organic extract was washed with brine and dried over Na₂SO₄, filtered and concentrated in vacuo. The crude product was purified by Isco chromatography (0 to 60% EtOAc/Hex) to give the title compound as a viscous oil that solidified on standing, 296 mg. LCMS m/z=327 [M+H]⁺

### Preparation 370

### 6-chloro-4-(1-hydroxypropan-2-yl)-2,7-naphthyridin-1(2H)-one

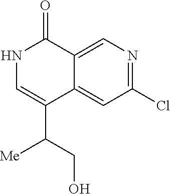 [SMILES: CC(CO)c1c[nH]c(=O)c2cnc(Cl)cc12]

The title compound was obtained, as an off-white solid, from 2-(1-(benzyloxy)-6-chloro-2,7-naphthyridin-4-yl)prop-2-en-1-ol (Preparation 369), following a similar procedure to that described in Preparation 296. LCMS m/z=239 [M+H]⁺

### Preparation 371

### (S)-2-(6-chloro-1-((R)-2-methylazetidin-1-yl)-2,7-naphthyridin-4-yl)propan-1-ol and (R)-2-(6-chloro-1-((S)-2-methylazetidin-1-yl)-2,7-naphthyridin-4-yl)propan-1-ol or (S)-2-(6-chloro-1-((S)-2-methylazetidin-1-yl)-2,7-naphthyridin-4-yl)propan-1-ol and (R)-2-(6-chloro-1-((R)-2-methylazetidin-1-yl)-2,7-naphthyridin-4-yl)propan-1-ol and

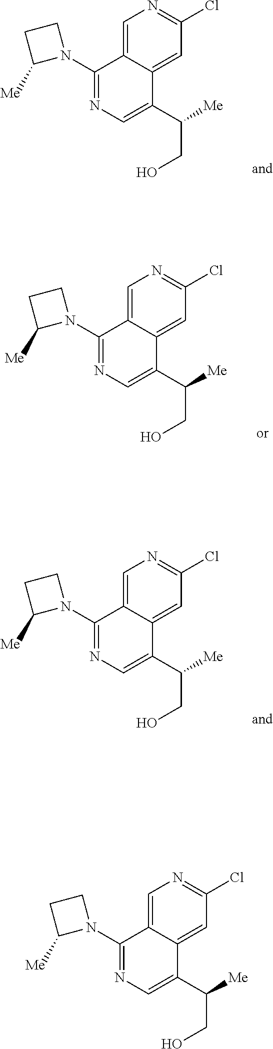 [SMILES: C[C@H](CO)c1cnc(N2CC[C@H]2C)c2cnc(Cl)cc12 ; C[C@@H](CO)c1cnc(N2CC[C@@H]2C)c2cnc(Cl)cc12 ; C[C@H](CO)c1cnc(N2CC[C@@H]2C)c2cnc(Cl)cc12 ; C[C@@H]1CCN1c1ncc([C@@H](C)CO)c2cc(Cl)ncc12]

A mixture of 6-chloro-4-(1-hydroxypropan-2-yl)-2,7-naphthyridin-1(2H)-one (Preparation 370, 209 mg, 0.876 mmol), 2-methylazetidine hydrochloride (113 mg, 1.05 mmol), DBU (400 mg, 2.63 mmol) and HBTU (503 mg, 1.14 mmol) in DMF 93 mL) was stirred at rt overnight. The mixture was diluted with water and extracted with EtOAc. The combined organic extracts were washed with water (2×) and brine, dried (Na₂SO₄) and concentrated in vacuo. The crude was purified by Isco chromatography (10-100% EtOAc/Hex) to give peak 1, (S)-2-(6-chloro-1-((R)-2-methylazetidin-1-yl)-2,7-naphthyridin-4-yl)propan-1-ol and (R)-2-(6-chloro-1-((S)-2-methylazetidin-1-yl)-2,7-naphthyridin-4-yl)propan-1-ol or (S)-2-(6-chloro-1-((S)-2-methylazetidin-1-yl)-2,7-naphthyridin-4-yl)propan-1-ol and (R)-2-(6-chloro-1-((R)-2-methylazetidin-1-yl)-2,7-naphthyridin-4-yl)propan-1-ol, 19 mg as a pale yellow solid

and peak 2, 75 mg. This was further purified by reverse phase Isco chromatography (0 to 20% MeCN/water containing 0.1% TFA). The clean fractions were combined and neutralized with NaHCO₃, then extracted with 10% MeOH/DCM (2×). The combined extracts were dried over Na₂SO₄ and evaporated under reduced pressure to afford additional title compound, 15 mg. LCMS m/z=290 [M+H]⁺

### Synthesis of Exemplary Compounds

### Example 1

### (3S,4R)-3-fluoro-1-(4-((5-isopropyl-8-((1r,3S)-3-((methylsulfonyl)methyl)cyclobutyl)isoquinolin-3-yl)amino)pyrimidin-2-yl)-3-methylpiperidin-4-ol

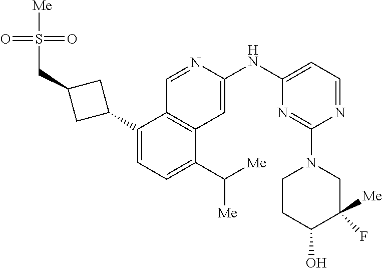 [SMILES: CC(C)c1ccc([C@H]2C[C@H](CS(C)(=O)=O)C2)c2cnc(Nc3ccnc(N4CC[C@@H](O)[C@@](C)(F)C4)n3)cc12]

A solution of (3S,4R)-1-(4-aminopyrimidin-2-yl)-3-fluoro-3-methylpiperidin-4-ol (Preparation 8, 20.1 mg, 0.089 mmol), 3-chloro-5-isopropyl-8-(3-((methylsulfonyl)methyl)cyclobutyl)isoquinoline (Preparation 134, 31.2 mg, 0.089 mmol), XPhos Pd G4 (3.81 mg, 4.43 μmol) and Cs₂CO₃ (57.8 mg, 0.177 mmol) in dioxane (0.5 mL) was purged with N₂ for 5 minutes before stirring at 90° C. for 1 h. Additional XPhos Pd G4 (3.81 mg, 4.43 μmol) was added and the reaction mixture heated overnight at 90° C. The solids were removed by filtration through a plug of Celite® and washed with 10% MeOH/DCM. The combined organics were evaporated to dryness in vacuo and the residue purified by ISCO (SiO₂, 0-10% MeOH/DCM) to afford the title compound as a white solid (25.6 mg, 53%). LCMS m/z=542 [M+H]⁺¹HNMR (400 MHz, DMSO-d₆) δ: 9.94 (s, 1H), 9.12 (s, 1H), 8.74 (s, 1H), 8.01 (d, 1H), 7.59 (d, 1H), 7.39 (d, 1H), 6.49 (t, 1H), 5.04 (d, 1H), 4.87-4.57 (m, 3H), 4.45 (p, 1H), 3.62 (dd, 4H), 3.31-3.06 (m, 2H), 2.97 (s, 1H), 2.80 (q, 1H), 2.12 (q, 1H), 1.77 (t, 3H), 1.45-1.33 (m, 12H).

### Example 2-23

The title compounds were prepared using an analogous method to that described for Example 1 using the appropriate chloride (RCl), (3S,4R)-1-(4-aminopyrimidin-2-yl)-3-fluoro-3-methylpiperidin-4-ol (Preparation 8) unless otherwise stated in the table and an appropriate catalyst as noted in the following table.

| Ex no | Name/Structure/RCl/Catalyst/HPLC/Data |
|---|---|
| 2 | 1-(3-((3-((2-((3S,4R)-3-fluoro-4-hydroxy-3-methylpiperidin-1-yl)pyrimidin-4- |
| | yl)amino)-5-isopropylisoquinolin-8-yl)methyl)azetidin-1-yl)ethan-1-one |
| | 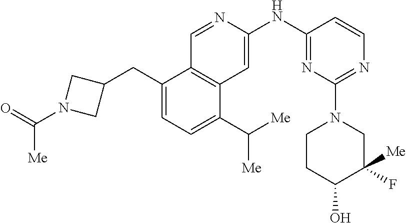 [SMILES: CC(=O)N1CC(Cc2ccc(C(C)C)c3cc(Nc4ccnc(N5CC[C@@H](O)[C@@](C)(F)C5)n4)ncc23)C1] |
| | RCl 1-(3-((3-chloro-5-isopropylisoquinolin-8-yl)methyl)azetidin-1-yl)ethan-1-one |
| | (Preparation 142); XPhos Pd G4, 90° C. |
| | RP-ISCO (0-60% MeCN/H2O (+0.1% TFA)) followed by neutralisation with NaHCO₃ |
| | and extraction into 5% MeOH/DCM |
| | White solid (30 mg, 57%. LCMS m/z = 507 [M + H]⁺ ¹HNMR (400 MHz, DMSO-d₆) δ: |
| | 9.93 (s, 1H), 9.30 (s, 1H), 8.70 (s, 1H), 7.97 (d, 1H), 7.50 (d, 1H), 7.22 (d, 1H), |
| | 6.45 (d, 1H), 5.00 (d, 1H), 4.79-4.61 (m, 2H), 4.16 (t, 1H), 3.95-3.82 (m, 2H), |
| | 3.64-3.48 (m, 3H), 3.39 (dd, 2H), 3.20-3.06 (m, 2H), 2.99 (ddd, 1H), 1.78-1.66 |
| | (m, 5H), 1.40-1.27 (m, 10H). |
| 3 | (3S,4R)-3-fluoro-1-(4-((5-isopropyl-8-(1-(methylsulfonyl)azetidin-3-yl)isoquinolin-3- |
| | yl)amino)pyrimidin-2-yl)-3-methylpiperidin-4-ol |
| | 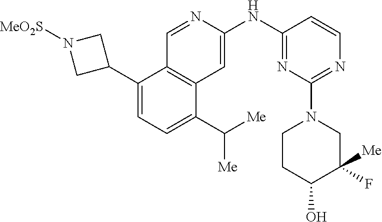 [SMILES: CC(C)c1ccc(C2CN(S(C)(=O)=O)C2)c2cnc(Nc3ccnc(N4CC[C@@H](O)[C@@](C)(F)C4)n3)cc12] |
| | RCl 3-chloro-5-isopropyl-8-(1-(methylsulfonyl)azetidin-3-yl)isoquinoline (Preparation |
| | 140); XPhos Pd G4, 90° C. |
| | RP-ISCO (0-60% MeCN/H2O (+0.1% TFA)) followed by neutralisation with NaHCO₃ |
| | and extraction into 5% MeOH/DCM |
| | White solid (26 mg, 64%. LCMS m/z = 529 [M + H]⁺ ¹HNMR (400 MHz, DMSO-d₆) δ: |
| | 9.95 (s, 1H), 9.05 (s, 1H), 8.72 (s, 1H), 7.98 (d, 1H), 7.58 (d, 1H), 7.44 (d, 1H), |
| | 6.46 (d, 1H), 5.00 (d, 1H), 4.79-4.60 (m, 3H), 4.43-4.34 (m, 2H), 4.20-4.09 (m, |
| | 2H), 3.67-3.47 (m, 2H), 3.22-3.07 (m, 2H), 3.04 (s, 3H), 1.79-1.65 (m, 2H), 1.42- |
| | 1.28 (m, 9H). |
| 4 | 1-(3-(3-((2-((3S,4R)-3-fluoro-4-hydroxy-3-methylpiperidin-1-yl)pyrimidin-4-yl)amino)- |
| | 5-isopropylisoquinolin-8-yl)azetidin-1-yl)ethan-1-one |
| | 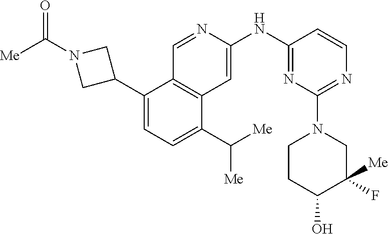 [SMILES: CC(=O)N1CC(c2ccc(C(C)C)c3cc(Nc4ccnc(N5CC[C@@H](O)[C@@](C)(F)C5)n4)ncc23)C1] |
| | RCl 1-(3-(3-chloro-5-isopropylisoquinolin-8-yl)azetidin-1-yl)ethan-1-one (Preparation |
| | 141); XPhos Pd G4, 90° C. |
| | RP-ISCO (0-60% MeCN/H2O (+0.1% TFA)) followed by neutralisation with NaHCO₃ |
| | and extraction into 5% MeOH/DCM |
| | White solid (28 mg, 64%. LCMS m/z = 493 [M + H]⁺ ¹HNMR (400 MHz, DMSO-d₆) δ: |
| | 9.94 (s, 1H), 9.03 (s, 1H), 8.72 (s, 1H), 7.98 (d, 1H), 7.58 (d, 1H), 7.44 (d, 1H), |
| | 6.47 (d, 1H), 5.00 (d, 1H), 4.79-4.56 (m, 4H), 4.39 (t, 1H), 4.32-4.25 (m, 1H), |
| | 4.08-4.02 (m, 1H), 3.66-3.47 (m, 2H), 3.21-3.05 (m, 2H), 1.79 (s, 3H), 1.77-1.67 |
| | (m, 2H), 1.41-1.28 (m, 9H). |
| 5 | (3S,4R)-3-fluoro-1-(4-((5-isopropyl-8-(5-methyl-1,3,4-oxadiazol-2-yl)-2,7- |
| | naphthyridin-3-yl)amino)pyrimidin-2-yl)-3-methylpiperidin-4-ol |
| | 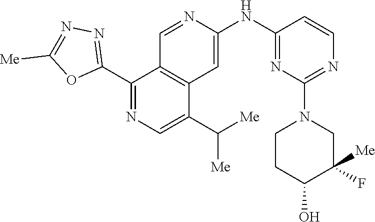 [SMILES: Cc1nnc(-c2ncc(C(C)C)c3cc(Nc4ccnc(N5CC[C@@H](O)[C@@](C)(F)C5)n4)ncc23)o1] |
| | RCl: 2-(6-chloro-4-isopropyl-2,7-naphthyridin-1-yl)-5-methyl-1,3,4-oxadiazole |
| | (Preparation 256); BrettPhos Pd G3, 100° C. |
| | HPLC-10: Gradient (% organic): 33-46% |
| | White solid (20 mg, 15%. LCMS m/z = 479 [M + H]⁺ ¹HNMR (300 MHz, DMSO-d₆) δ: |
| | 10.49 (s, 1H), 10.29 (s, 1H), 8.79 (s, 1H), 8.68 (s, 1H), 8.07 (d, 1H), 6.48 (d, 1H), |
| | 5.06 (d, 1H), 4.71 (dd, 2H), 3.75-3.49 (m, 2H), 3.26-3.02 (m, 2H), 2.67 (s, 3H), |
| | 1.86-1.63 (m, 2H), 1.52-1.26 (m, 9H). |
| 6 | (3S,4R)-3-fluoro-1-(4-((5-isopropyl-8-(1-methyl-1H-1,2,4-triazol-3-yl)-2,6- |
| | naphthyridin-3-yl)amino)pyrimidin-2-yl)-3-methylpiperidin-4-ol |
| | 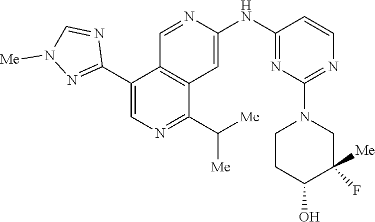 [SMILES: CC(C)c1ncc(-c2ncn(C)n2)c2cnc(Nc3ccnc(N4CC[C@@H](O)[C@@](C)(F)C4)n3)cc12] |
| | RCl: 7-chloro-1-isopropyl-4-(1-methyl-1H-1,2,4-triazol-3-yl)-2,6-naphthyridine |
| | (Preparation 357). BrettPhos Pd G3, 120° C. |
| | HPLC-8: Gradient (% organic): 30-42% |
| | White solid (9 mg, 18%. LCMS m/z = 478 [M + H]⁺ ¹HNMR (300 MHz, DMSO-d₆) δ: |
| | 10.19 (d, 1H), 8.96 (d, 2H), 8.60 (d, 1H), 8.02 (d, 1H), 6.41 (d, 1H), 4.76 (dd, |
| | 2H), 4.12 (d, 3H), 4.04 (p, 1H), 3.73 (ddd, 1H), 3.39 (m, 2H), 2.04-1.90 (m, 2H), |
| | 1.56-1.43 (m, 9H). |
| 7 | (3S,4R)-3-fluoro-1-(4-((5-isopropyl-8-(5-methyl-1,3,4-oxadiazol-2-yl)-2,6- |
| | naphthyridin-3-yl)amino)pyrimidin-2-yl)-3-methylpiperidin-4-ol |
| | 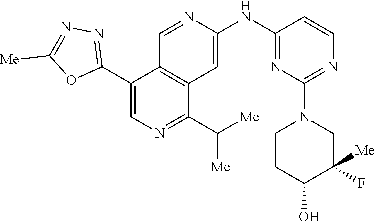 [SMILES: Cc1nnc(-c2cnc(C(C)C)c3cc(Nc4ccnc(N5CC[C@@H](O)[C@@](C)(F)C5)n4)ncc23)o1] |
| | RCl: 2-(7-chloro-1-isopropyl-2,6-naphthyridin-4-yl)-5-methyl-1,3,4-oxadiazole |
| | (Preparation 358). BrettPhos Pd G3, 120° C. |
| | HPLC-8: Gradient (% organic): 35-45% |
| | White solid (17 mg, 25%. LCMS m/z = 479 [M + H]⁺ ¹HNMR (300 MHz, CDCl₃) δ: |
| | 10.41 (s, 1H), 10.16 (s, 1H), 9.16 (s, 1H), 8.97 (s, 1H), 8.93 (s, 1H), 8.07 (d, 1H), |
| | 6.52 (d, 1H), 5.07 (d, 1H), 4.80-4.60 (m, 3H), 3.97 (p, 1H), 3.70-3.50 (m, 1H), |
| | 3.30-3.10 (m, 3H), 2.74 (s, 3H), 1.80-1.70 (m, 2H), 1.45-1.30 (6H). |
| 8 | (3S,4R)-3-fluoro-1-(4-((5-isopropyl-8-(5-methyl-1,2,4-oxadiazol-3-yl)-2,6- |
| | naphthyridin-3-yl)amino)pyrimidin-2-yl)-3-methylpiperidin-4-ol |
| | 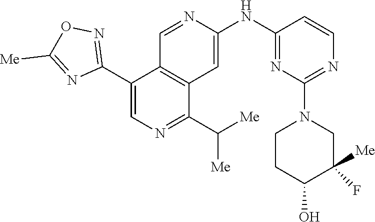 [SMILES: Cc1nc(-c2cnc(C(C)C)c3cc(Nc4ccnc(N5CC[C@@H](O)[C@@](C)(F)C5)n4)ncc23)no1] |
| | RCl: 3-(7-chloro-1-isopropyl-2,6-naphthyridin-4-yl)-5-methyl-1,2,4-oxadiazole |
| | (Preparation 362). XantPhos Pd G2, 100° C. |
| | HPLC-10: Gradient (% organic): 69-79% |
| | Yellow solid (4.7 mg, 14%. LCMS m/z = 479 [M + H]⁺ ¹HNMR (400 MHz, CDCl₃) δ: |
| | 10.10 (s, 1H), 9.16 (s, 1H), 8.91 (s, 1H), 8.14 (d, 1H), 7.89-7.58 (m, 1H), 6.21 (s, |
| | 1H), 4.74-4.42 (m, 2H), 3.98 (p, 1H), 3.78 (d, 1H), 3.69-3.49 (m, 2H), 2.77 (s, |
| | 3H), 2.18-1.92 (m, 2H), 1.56 (d, 3H), 1.50 (d, 6H). |
| 9 | (3S,4R)-3-fluoro-1-(4-((5-isopropyl-8-(1-methyl-1H-pyrazol-4-yl)-2,7-naphthyridin-3- |
| | yl)amino)pyrimidin-2-yl)-3-methylpiperidin-4-ol |
| | 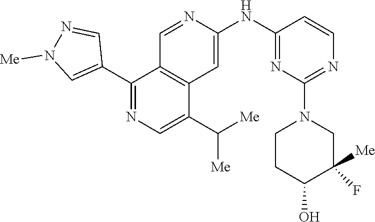 [SMILES: CC(C)c1cnc(-c2cnn(C)c2)c2cnc(Nc3ccnc(N4CC[C@@H](O)[C@@](C)(F)C4)n3)cc12] |
| | RCl: 6-chloro-4-isopropyl-1-(1-methyl-1H-pyrazol-4-yl)-2,7-naphthyridine (Preparation |
| | 261). BrettPhos Pd G3, 100° C. |
| | HPLC-18, Gradient (% organic): 25-45% |
| | White solid (13 mg, 25%. LCMS m/z = 477 [M + H]⁺ ¹HNMR (300 MHz, DMSO-d₆) δ: |
| | 10.25 (s, 1H), 9.45 (s, 1H), 8.67 (s, 1H), 8.48 (s, 1H), 8.39 (s, 1H), 8.14-7.89 (m, |
| | 2H), 6.50 (d, 1H), 5.04 (d, 1H), 4.89-4.41 (m, 2H), 3.96 (s, 3H), 3.55 (dp, 2H), |
| | 3.24-3.03 (m, 2H), 1.89-1.67 (m, 2H), 1.50-1.26 (m, 9H). |
| 10 | (3S,4R)-3-fluoro-1-(4-((5-isopropyl-8-(1-methyl-1H-1,2,3-triazol-4-yl)-2,7- |
| | naphthyridin-3-yl)amino)pyrimidin-2-yl)-3-methylpiperidin-4-ol |
| | 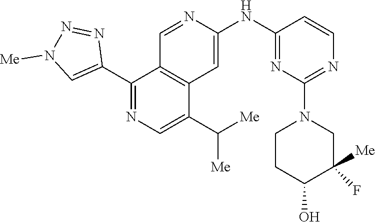 [SMILES: CC(C)c1cnc(-c2cn(C)nn2)c2cnc(Nc3ccnc(N4CC[C@@H](O)[C@@](C)(F)C4)n3)cc12] |
| | RCl: 6-chloro-4-isopropyl-1-(1-methyl-1H-1,2,3-triazol-4-yl)-2,7-naphthyridine |
| | (Preparation 262). BrettPhos Pd G3, 130° C. microwave |
| | HPLC-9: Gradient (% organic): 30-57% |
| | White solid (8 mg, 5%. LCMS m/z = 478 [M + H]⁺ ¹HNMR (300 MHz, CDCl₃) δ: 10.67 |
| | (s, 1H), 8.68 (s, 1H), 8.50 (s, 1H), 8.16 (d, 1H), 7.54 (s, 5H), 6.24 (s, 1H), 4.60 (s, |
| | 1H), 4.46 (s, 1H), 3.87-3.74 (m, 1H), 3.71-3.43 (m, 3H), 2.10-1.82 (m, 3H), 1.64- |
| | 1.37 (m, 9H). |
| 11 | (3S,4R)-3-fluoro-1-(4-((5-isopropyl-8-(1-methyl-1H-pyrazol-3-yl)-2,6-naphthyridin-3- |
| | yl)amino)pyrimidin-2-yl)-3-methylpiperidin-4-ol |
| | 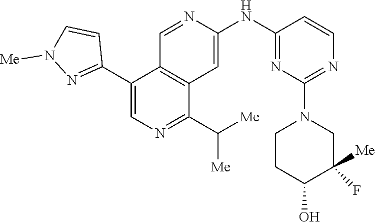 [SMILES: CC(C)c1ncc(-c2ccn(C)n2)c2cnc(Nc3ccnc(N4CC[C@@H](O)[C@@](C)(F)C4)n3)cc12] |
| | RCl: 7-chloro-1-isopropyl-4-(1-methyl-1H-pyrazol-3-yl)-2,6-naphthyridine (Preparation |
| | 353). BrettPhos Pd G3, 120° C. |
| | HPLC-18, Gradient (% organic): 29-44%) |
| | White solid (30 mg, 52%. LCMS m/z = 477 [M + H]⁺ ¹HNMR (300 MHz, DMSO-d₆) δ: |
| | 10.18 (s, 1H), 10.00 (d, 1H), 8.87 (s, 1H), 8.64 (s, 1H), 8.04 (d, 1H), 7.91 (d, 1H), |
| | 6.83 (d, 1H), 6.48 (d, 1H), 5.04 (d, 1H), 4.88-4.60 (m, 2H), 4.01 (s, 3H), 3.89 (p, |
| | 1H), 3.57 (dt, 1H), 3.25-3.08 (m, 2H), 1.90-1.73 (m, 2H), 1.49-1.31 (m, 9H). |
| 12 | (3S,4R)-3-fluoro-1-(4-((5-isopropyl-8-(1-methyl-1H-1,2,3-triazol-4-yl)-2,6- |
| | naphthyridin-3-yl)amino)pyrimidin-2-yl)-3-methylpiperidin-4-ol |
| | 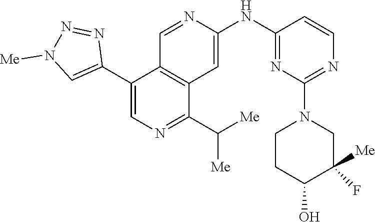 [SMILES: CC(C)c1ncc(-c2cn(C)nn2)c2cnc(Nc3ccnc(N4CC[C@@H](O)[C@@](C)(F)C4)n3)cc12] |
| | RCl: 7-chloro-1-isopropyl-4-(1-methyl-1H-1,2,3-triazol-4-yl)-2,6-naphthyridine |
| | (Preparation 354). BrettPhos Pd G3, 130° C. microwave |
| | HPLC-9: Gradient (% organic): 30-57%) |
| | White solid (9 mg, 11%. LCMS m/z = 478 [M + H]⁺ ¹HNMR (300 MHz, DMSO-d₆) δ: |
| | 10.27 (s, 1H), 9.82 (s, 1H), 8.90 (s, 1H), 8.70 (s, 1H), 8.62 (s, 1H), 8.03 (d, 1H), |
| | 6.45 (d, 1H), 5.05 (d, 1H), 4.86-4.58 (m, 2H), 4.18 (s, 3H), 3.91 (dq, 1H), 3.57 |
| | (dq, 1H), 3.26-3.02 (m, 2H), 1.87-1.75 (m, 2H), 1.50-1.30 (m, 9H). |
| 13 | (3S,4R)-1-(4-((8-(5-((dimethylamino)methyl)-1-methyl-1H-pyrazol-4-yl)-5-isopropyl- |
| | 2,7-naphthyridin-3-yl)amino)pyrimidin-2-yl)-3-fluoro-3-methylpiperidin-4-ol |
| | trifluoroacetate |
| | 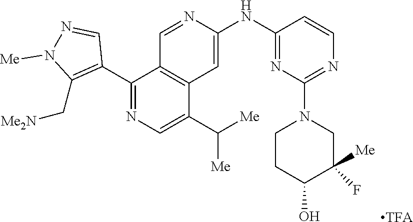 [SMILES: CC(C)c1cnc(-c2cnn(C)c2CN(C)C)c2cnc(Nc3ccnc(N4CC[C@@H](O)[C@@](C)(F)C4)n3)cc12] |
| | RCl: 1-(4-(6-chloro-4-isopropyl-2,7-naphthyridin-1-yl)-1-methyl-1H-pyrazol-5-yl)- |
| | N,N-dimethylmethanamine (Preparation 264). BrettPhos Pd G3, 100° C. |
| | HPLC-19, Gradient (% organic): 25-50% |
| | White solid (94 mg, 38%. LCMS m/z = 534 [M + H]⁺ ¹HNMR (400 MHz, DMSO-d₆) δ: |
| | 10.29 (s, 1H), 9.17 (s, 1H), 8.70 (s, 1H), 8.54 (s, 1H), 8.05 (d, 1H), 7.79 (s, 1H), |
| | 6.48 (d, 1H), 5.05 (d, 1H), 4.73 (dd, 2H), 3.96 (s, 3H), 3.77-3.46 (m, 4H), 3.25- |
| | 3.00 (m, 2H), 2.01 (s, 6H), 1.84-1.71 (m, 2H), 1.52-1.19 (m, 9H). |
| 14 | (3S,4R)-3-fluoro-1-(4-((5-isopropyl-8-(1-methyl-1H-pyrazol-4-yl)-2,6-naphthyridin-3- |
| | yl)amino)pyrimidin-2-yl)-3-methylpiperidin-4-ol |
| | 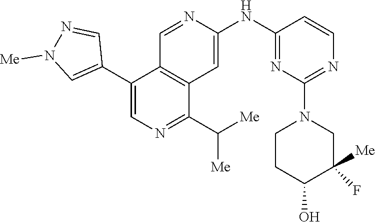 [SMILES: CC(C)c1ncc(-c2cnn(C)c2)c2cnc(Nc3ccnc(N4CC[C@@H](O)[C@@](C)(F)C4)n3)cc12] |
| | RCl: 7-chloro-1-isopropyl-4-(1-methyl-1H-pyrazol-4-yl)-2,6-naphthyridine (Preparation |
| | 355). BrettPhos Pd G3, 120° C. |
| | HPLC-18, Gradient (% organic): 30-40% |
| | Yellow solid (27 mg, 47%. LCMS m/z = 477 [M + H]⁺ ¹HNMR (300 MHz, DMSO-d₆) δ: |
| | 10.21 (s, 1H), 9.32 (s, 1H), 8.87 (s, 1H), 8.40 (s, 1H), 8.24 (s, 1H), 8.04 (d, 1H), |
| | 7.88 (s, 1H), 6.47 (d, 1H), 5.04 (d, 1H), 4.83-4.64 (m, 2H), 3.97 (s, 3H), 3.88 (p, |
| | 1H), 3.57 (dt, 1H), 3.24-3.08 (m, 2H), 1.80-1.73 (m, 2H), 1.45-1.32 (m, 9H). |
| 15 | (3S,4R)-3-fluoro-1-(4-((5-isopropyl-8-(1-methyl-1H-1,2,4-triazol-3-yl)-2,7- |
| | naphthyridin-3-yl)amino)pyrimidin-2-yl)-3-methylpiperidin-4-ol |
| | 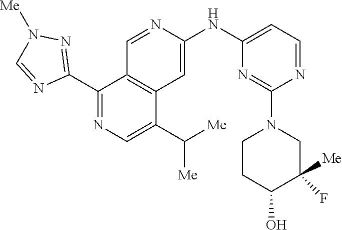 [SMILES: CC(C)c1cnc(-c2ncn(C)n2)c2cnc(Nc3ccnc(N4CC[C@@H](O)[C@@](C)(F)C4)n3)cc12] |
| | RCl: 6-chloro-4-isopropyl-1-(1-methyl-1H-1,2,4-triazol-3-yl)-2,7-naphthyridine |
| | (Preparation 260). BrettPhos Pd G3, 100° C. |
| | HPLC-13, Gradient (% organic): 29-40% |
| | Yellow solid (10 mg, 17.5%. LCMS m/z = 478 [M + H]⁺ ¹HNMR (300 MHz, DMSO-d₆) |
| | δ: 10.32 (s, 1H), 10.01 (s, 1H), 8.72 (s, 2H), 8.59 (s, 1H), 8.05 (d, 1H), 6.48 (d, |
| | 1H), 5.05 (d, 1H), 4.70 (s, 2H), 4.03 (s, 3H), 3.61 (m, 2H), 3.19 (m, 2H), 1.75 (s, |
| | 2H), 1.52-1.28 (m, 9H). |
| 16 | (3S,4R)-1-(4-((8-(1-(difluoromethyl)-1H-pyrazol-4-yl)-5-isopropyl-2,7-naphthyridin-3- |
| | yl)amino)pyrimidin-2-yl)-3-fluoro-3-methylpiperidin-4-ol |
| | 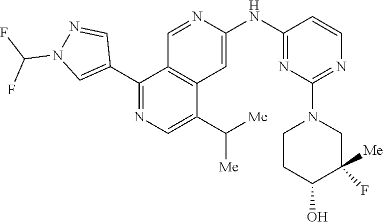 [SMILES: CC(C)c1cnc(-c2cnn(C(F)F)c2)c2cnc(Nc3ccnc(N4CC[C@@H](O)[C@@](C)(F)C4)n3)cc12] |
| | RCl: 6-chloro-1-(1-(difluoromethyl)-1H-pyrazol-4-yl)-4-isopropyl-2,7-naphthyridine |
| | (Preparation 265). BrettPhos Pd G3, 100° C. |
| | HPLC-18, Gradient (% organic): 33-45% |
| | White solid (95 mg, 60%. LCMS m/z = 513 [M + H]⁺ ¹HNMR (300 MHz, DMSO-d₆) δ: |
| | 10.32 (s, 1H), 9.43 (d, 1H), 8.94 (d, 1H), 8.73 (s, 1H), 8.56 (s, 1H), 8.37 (s, 1H), |
| | 8.07 (d, 1H), 7.94 (t, 1H), 6.53 (d, 1H), 5.07 (d, 1H), 4.85-4.60 (m, 2H), 3.74-3.44 |
| | (m, 2H), 3.26-3.03 (m, 2H), 1.85-1.66 (m, 2H), 1.57-1.32 (m, 9H). |
| 17 | (3S,4R)-1-(4-((8-(1,5-dimethyl-1H-1,2,3-triazol-4-yl)-5-isopropyl-2,7-naphthyridin-3- |
| | yl)amino)pyrimidin-2-yl)-3-fluoro-3-methylpiperidin-4-ol |
| | 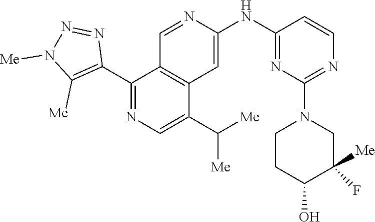 [SMILES: Cc1c(-c2ncc(C(C)C)c3cc(Nc4ccnc(N5CC[C@@H](O)[C@@](C)(F)C5)n4)ncc23)nnn1C] |
| | RCl: 6-chloro-1-(1,5-dimethyl-1H-1,2,3-triazol-4-yl)-4-isopropyl-2,7-naphthyridine |
| | (Preparation 266). BrettPhos Pd G3, 100° C. |
| | HPLC-11: Gradient (% organic): 34-46% |
| | White solid (32 mg, 24%. LCMS m/z = 492 [M + H]⁺ ¹HNMR (300 MHz, DMSO-d₆) δ: |
| | 10.36 (s, 1H), 10.01 (s, 1H), 8.74 (m, 1H), 8.60 (s, 1H), 8.07 (s, 1H), 8.05 (d, 1H), |
| | 6.48 (s, 1H), 5.08 (s, 1H), 4.79-4.67 (d, 2H), 4.08 (s, 3H), 3.60-3.54 (d, 2H), 3.19 |
| | (d, 2H), 2.60 (s, 3H), 1.77 (m, 1H), 1.43 (d, 9H). |
| 18 | (3S,4R)-3-fluoro-1-(4-((5-isopropyl-8-(1-(methylsulfonyl)-1H-pyrazol-4-yl)-2,7- |
| | naphthyridin-3-yl)amino)pyrimidin-2-yl)-3-methylpiperidin-4-ol |
| | 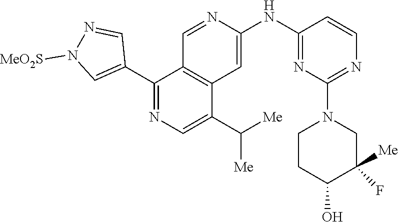 [SMILES: CC(C)c1cnc(-c2cnn(S(C)(=O)=O)c2)c2cnc(Nc3ccnc(N4CC[C@@H](O)[C@@](C)(F)C4)n3)cc12] |
| | RCl: 6-chloro-4-isopropyl-1-(1-(methylsulfonyl)-1H-pyrazol-4-yl)-2,7-naphthyridine |
| | (Preparation 267). BrettPhos Pd G3, 80° C. |
| | HPLC-1: Gradient (% organic): 30-48% |
| | White solid. LCMS m/z = 541 [M + H]⁺ ¹HNMR (400 MHz, DMSO-d₆) δ: 10.32 (s, 1H), |
| | 9.47 (s, 1H), 8.89 (s, 1H), 8.73 (s, 1H), 8.58 (s, 1H), 8.50 (s, 1H), 8.08 (d, 1H), |
| | 6.55 (d, 1H), 5.08 (d, 1H), 4.75 (dd, 2H), 3.72 (s, 3H), 3.63-3.50 (m, 2H), 3.25- |
| | 3.04 (m, 2H), 1.87-1.63 (m, 2H), 1.51-1.26 (m, 9H). |
| 19 | (3S,4R)-3-fluoro-1-(4-((5-isopropyl-8-((1-methyl-1H-pyrazol-4-yl)ethynyl)-2,7- |
| | naphthyridin-3-yl)amino)pyrimidin-2-yl)-3-methylpiperidin-4-ol |
| | 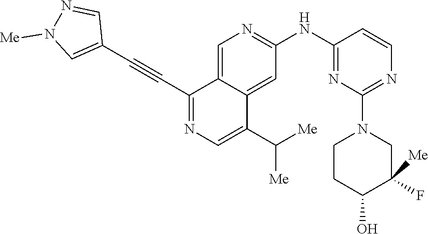 [SMILES: CC(C)c1cnc(C#Cc2cnn(C)c2)c2cnc(Nc3ccnc(N4CC[C@@H](O)[C@@](C)(F)C4)n3)cc12] |
| | RCl: 6-chloro-4-isopropyl-1-((1-methyl-1H-pyrazol-4-yl)ethynyl)-2,7-naphthyridine |
| | (Preparation 277). RuPhos Pd G3, 130° C. |
| | HPLC-28, Gradient (% organic): 31-42% |
| | Yellow solid. LCMS m/z = 501 [M + H]⁺ ¹HNMR (300 MHz, DMSO-d₆) δ: 10.35 (s, |
| | 1H), 9.53 (d, 1H), 8.71 (s, 1H), 8.50 (s, 1H), 8.35 (s, 1H), 8.07 (d, 1H), 7.95 (d, |
| | 1H),6.52 (d, 1H), 5.06 (d, 1H), 4.70 (s, 2H), 3.91 (s, 3H), 3.53 (s, 2H),3.06 (s, |
| | 2H), 1.75 (s, 2H), 1.47-1.38 (m, 9H). |
| 20 | (3S,4R)-3-fluoro-1-(4-((5-isopropyl-8-(1-(oxetan-3-yl)-1H-imidazol-4-yl)-2,7- |
| | naphthyridin-3-yl)amino)pyrimidin-2-yl)-3-methylpiperidin-4-ol |
| | 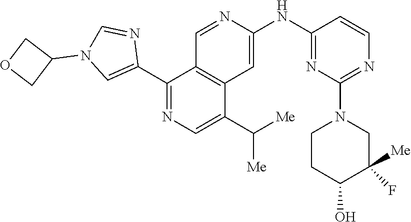 [SMILES: CC(C)c1cnc(-c2cn(C3COC3)cn2)c2cnc(Nc3ccnc(N4CC[C@@H](O)[C@@](C)(F)C4)n3)cc12] |
| | RCl: 6-chloro-4-isopropyl-1-(1-(oxetan-3-yl)-1H-imidazol-4-yl)-2,7-naphthyridine |
| | (Preparation 269). BrettPhos Pd G4, 100° C. |
| | HPLC-15, Gradient (% organic): 15-45% |
| | Yellow solid (2.8 mg, 7.4%). LCMS m/z = 519 [M + H]⁺ ¹HNMR (400 MHz, CDCl₃) δ: |
| | 10.57 (s, 1H), 8.61 (s, 1H), 8.51 (s, 1H), 8.14-8.10 (m, 2H), 7.80 (s, 1H), 7.57 (s, |
| | 1H), 6.20 (d, 1H), 5.46-5.39 (m, 1H), 5.20-5.16 (m, 2H), 5.03-4.98 (m, 2H), 4.63- |
| | 4.55 (m, 1H), 4.49-4.41 (m, 1H), 3.82-3.72 (m, 1H), 3.67-3.57 (m, 3H), 2.08-2.01 |
| | (m, 1H), 2.00-1.93 (m, 2H), 1.56-1.50 (m, 3H), 1.47 (d, 6H). |
| 21 | (3S,4R)-3-fluoro-1-(4-((5-isopropyl-8-(1-(oxetan-3-yl)-1H-pyrazol-3-yl)-2,7- |
| | naphthyridin-3-yl)amino)pyrimidin-2-yl)-3-methylpiperidin-4-ol |
| | 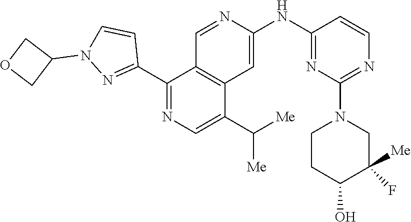 [SMILES: CC(C)c1cnc(-c2ccn(C3COC3)n2)c2cnc(Nc3ccnc(N4CC[C@@H](O)[C@@](C)(F)C4)n3)cc12] |
| | RCl: 6-chloro-4-isopropyl-1-(1-(oxetan-3-yl)-1H-pyrazol-3-yl)-2,7-naphthyridine |
| | (Preparation 270). BrettPhos Pd G4, 100° C. |
| | HPLC-15, Gradient (% organic): 20-50% |
| | Yellow solid (17 mg, 51%). LCMS m/z = 518 [M + H]⁺ ¹HNMR (400 MHz, CDCl₃) δ: |
| | 10.38 (s, 1H), 8.64 (s, 1H), 8.59 (s, 1H), 8.14 (d, 1H), 7.72 (d, 1H), 7.53-7.47 (m, |
| | 1H), 7.16-7.04 (m, 1H), 6.24-6.19 (m, 1H), 5.66-5.60 (m, 1H), 5.26-5.22 (m, 2H), |
| | 5.16-5.11 (m, 2H), 4.64-4.56 (m, 1H), 4.51-4.42 (m, 1H), 3.82-3.72 (m, 1H), |
| | 3.67-3.58 (m, 3H), 2.03-1.95 (m, 3H), 1.51 (s, 3H), 1.50-1.47 (m, 6H). |
| 22 | (3S,4R)-3-fluoro-1-(4-((5-isopropyl-8-(1-(1-methylazetidin-3-yl)-1H-pyrazol-4-yl)-2,7- |
| | naphthyridin-3-yl)amino)pyrimidin-2-yl)-3-methylpiperidin-4-ol |
| | 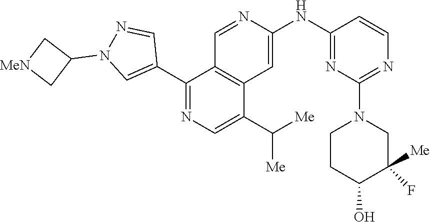 [SMILES: CC(C)c1cnc(-c2cnn(C3CN(C)C3)c2)c2cnc(Nc3ccnc(N4CC[C@@H](O)[C@@](C)(F)C4)n3)cc12] |
| | RCl: 6-chloro-4-isopropyl-1-(1-(1-methylazetidin-3-yl)-1H-pyrazol-4-yl)-2,7- |
| | naphthyridine formate (Preparation 273). XPhos G2, 90° C. |
| | HPLC-14, Gradient (% organic): 20-45% |
| | Yellow solid (16 mg, 38%). LCMS m/z = 532 [M + H]⁺ ¹HNMR (400 MHz, MeOH-d₄) |
| | δ: 9.46 (s, 1H), 8.76 (s, 1H), 8.43 (s, 1H), 8.37 (s, 1H), 8.10 (s, 1H), 8.05 (d, 1H), |
| | 6.45 (d, 1H), 5.23 (m, 1H), 4.78-4.73 (m, 2H), 3.99-3.95 (m, 2H), 3.79-3.76 (m, |
| | 2H), 3.75-3.69 (m, 2H), 3.36 (s, 1H), 3.29-3.26 (m, 1H), 2.55 (s, 3H), 1.98-1.89 |
| | (m, 2H), 1.52-1.47 (m, 9H). |
| 23 | N-(2-(1-oxa-7-azaspiro[3.5]nonan-7-yl)pyrimidin-4-yl)-5-isopropyl-8-(1-methyl-1H- |
| | 1,2,3-triazol-4-yl)-2,7-naphthyridin-3-amine |
| | 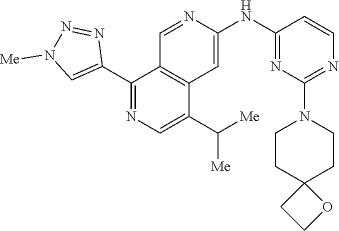 [SMILES: CC(C)c1cnc(-c2cn(C)nn2)c2cnc(Nc3ccnc(N4CCC5(CCO5)CC4)n3)cc12] |
| | RCl: 6-chloro-4-isopropyl-1-(1-methyl-1H-1,2,3-triazol-4-yl)-2,7-naphthyridine |
| | (Preparation 262); RNH₂: 2-(1-oxa-7-azaspiro[3.5]nonan-7-yl)pyrimidin-4-amine |
| | (Preparation 62). BrettPhos Pd G3, 100° C. |
| | HPLC-18, Gradient (% organic): 40-60% |
| | Yellow solid (9 mg, 7%). LCMS m/z = 472 [M + H]⁺ ¹HNMR (300 MHz, DMSO-d₆) δ: |
| | 10.43 (d, 1H), 10.35 (s, 1H), 8.76 (s, 1H), 8.73 (s, 1H), 8.54 (s, 1H), 8.05 (d, 1H), |
| | 6.47 (d), 4.45 (t, 2H), 4.18 (s, 3H), 3.83 (q, 4H), 3.52 (p, 1H), 2.46-2.38 (m, 2H), |
| | 1.85 (s, 5H), 1.43 (d, 6H). |

### Example 24

### (3S,4R)-3-fluoro-1-(4-((5-isopropyl-8-(5-methyl-1H-1,2,4-triazol-3-yl)-2,7-naphthyridin-3-yl)amino)pyrimidin-2-yl)-3-methylpiperidin-4-ol

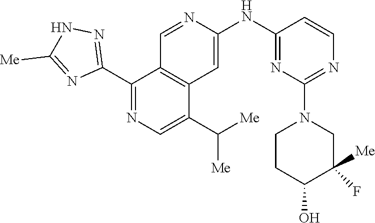 [SMILES: Cc1nc(-c2ncc(C(C)C)c3cc(Nc4ccnc(N5CC[C@@H](O)[C@@](C)(F)C5)n4)ncc23)n[nH]1]

Part 1. A mixture of 6-chloro-4-isopropyl-1-(5-methyl-4-((2-(trimethylsilyl)ethoxy)methyl)-4H-1,2,4-triazol-3-yl)-2,7-naphthyridine (Preparation 257, 75.2 mg, 0.180 mmol), (3S,4R)-1-(4-aminopyrimidin-2-yl)-3-fluoro-3-methylpiperidin-4-ol (Preparation 8, 40.7 mg, 0.180 mmol), BrettPhos Pd G3 (16.2 mg, 0.018 mmol) and Cs₂CO₃ (146 mg, 0.450 mmol) in dioxane was heated to 100° C. for 3 h. The reaction mixture was evaporated to dryness and the residue purified by prep-TLC (20:1 DCM/MeOH) to afford (3S,4R)-3-fluoro-1-(4-((5-isopropyl-8-(5-methyl-1-((2-(trimethylsilyl)ethoxy)methyl)-1H-1,2,4-triazol-3-yl)-2,7-naphthyridin-3-yl)amino)pyrimidin-2-yl)-3-methylpiperidin-4-ol as a yellow solid (55 mg, 50%). 608 [M+H]⁺

Part 2. The compound of Part 1 in DCM (1 mL) was treated with TFA (1 mL) and the resulting mixture was stirred at rt for 2 h. The mixture was evaporated to dryness and the reside purified by prep-HPLC-18 (Gradient (% organic) 20-35%) to afford the title compound as a yellow solid (10 mg, 21%). LCMS m/z=478 [M+H]⁺¹HNMR (300 MHz, DMSO-d₆) δ:14.53 (s, 1H), 10.67 (s, 1H), 10.36 (s, 1H), 8.73 (s, 1H), 8.58 (s, 1H), 8.05 (d, 1H), 6.50 (d, 1H), 5.05 (d, 1H), 4.84-4.51 (m, 2H), 3.69-3.43 (m, 2H), 3.26-3.01 (m, 2H), 2.48 (d, 3H), 1.75 (s, 2H), 1.50-1.27 (m, 9H).

### Example 25

### (3S,4R)-3-fluoro-1-(4-((5-isopropyl-8-(1H-1,2,4-triazol-3-yl)-2,7-naphthyridin-3-yl)amino)pyrimidin-2-yl)-3-methylpiperidin-4-ol

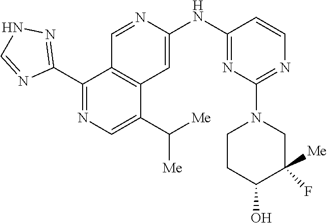 [SMILES: CC(C)c1cnc(-c2nc[nH]n2)c2cnc(Nc3ccnc(N4CC[C@@H](O)[C@@](C)(F)C4)n3)cc12]

The title compound was prepared from 6-chloro-4-isopropyl-1-(1-((2-(trimethylsilyl)ethoxy)methyl)-1H-1,2,4-triazol-5-yl)-2,7-naphthyridine (Preparation 258) and (3S,4R)-1-(4-aminopyrimidin-2-yl)-3-fluoro-3-methylpiperidin-4-ol (Preparation 8) using an analogous 2-part process as described for Example 24. Prep-HPLC-13 (Gradient (% organic): 20-35%) to afford the title compound as a yellow solid (10 mg, 36%). LCMS m/z=464 [M+H]⁺¹HNMR (300 MHz, DMSO-d₆) δ: 14.91 (s, 1H), 10.71 (s, 1H), 10.42 (s, 1H), 8.76 (s, 1H), 8.60 (s, 1H), 8.24 (s, 1H), 8.05 (d, 1H), 6.47 (d, 1H), 5.06 (d, 1H), 4.80-4.60 (m, 2H), 3.69-3.45 (m, 2H), 3.18 (s, 2H), 1.75 (s, 2H), 1.51-1.28 (m, 9H).

### Example 26

### (3S,4R)-3-fluoro-1-(4-((5-isopropyl-8-(5-methyl-1H-1,2,4-triazol-3-yl)isoquinolin-3-yl)amino)pyrimidin-2-yl)-3-methylpiperidin-4-ol

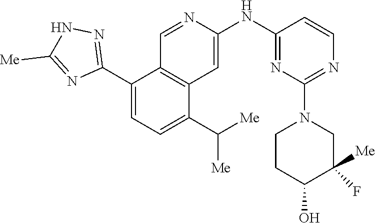 [SMILES: Cc1nc(-c2ccc(C(C)C)c3cc(Nc4ccnc(N5CC[C@@H](O)[C@@](C)(F)C5)n4)ncc23)n[nH]1]

The title compound was prepared from 3-chloro-5-isopropyl-8-(5-methyl-1-((2-(trimethylsilyl)ethoxy)methyl)-1H-1,2,4-triazol-3-yl)isoquinoline (Preparation 144) and (3S,4R)-1-(4-aminopyrimidin-2-yl)-3-fluoro-3-methylpiperidin-4-ol (Preparation 8) using an analogous 2-part process as described for Example 24. Prep-HPLC-18 (Gradient (% organic) 25-37%) to afford the title compound as a yellow solid (10 mg, 21%). LCMS m/z=477 [M+H]⁺¹HNMR (300 MHz, DMSO-d₆) δ: 10.20 (s, 1H), 10.01 (s, 1H), 8.77 (s, 1H), 7.98 (t, 2H), 7.68 (d, 1H), 6.46 (d, 1H), 5.04 (d, 1H), 4.81-4.57 (m, 2H), 3.73-3.44 (m, 2H), 3.15 (d, 2H), 2.47 (s, 3H), 1.74 (s, 2H), 1.44-1.26 (m, 9H).

### Example 27

### (3S,4R)-3-fluoro-1-(4-((8-(5-(2-hydroxypropan-2-yl)-1,3,4-oxadiazol-2-yl)-5-isopropyl-2,7-naphthyridin-3-yl)amino)pyrimidin-2-yl)-3-methylpiperidin-4-ol

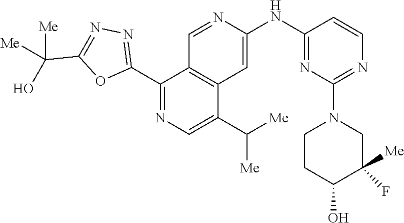 [SMILES: CC(C)c1cnc(-c2nnc(C(C)(C)O)o2)c2cnc(Nc3ccnc(N4CC[C@@H](O)[C@@](C)(F)C4)n3)cc12]

Part 1. A mixture of 2-(5-(6-chloro-4-isopropyl-2,7-naphthyridin-1-yl)-1,3,4-oxadiazol-2-yl)propan-2-yl acetate (Preparation 276, 200 mg, 0.533 mmol), (3S,4R)-1-(4-aminopyrimidin-2-yl)-3-fluoro-3-methylpiperidin-4-ol (Preparation 8, 120 mg, 0.533 mmol), BrettPhos Pd G3 (48.2 mg, 0.053 mmol) and Cs₂CO₃ (345 mg, 1.06 mmol) in dioxane (5 mL) was heated under N₂ at 100° C. for 3 h. The reaction mixture was diluted with EtOAc (100 mL) and washed with brine (2×50 mL). The combined organics were dried (Na₂SO₄) and evaporated to dryness in vacuo. The residue was purified by column chromatography (20:1 DCM/MeOH) to afford 2-(5-(6-((2-((3S,4R)-3-fluoro-4-hydroxy-3-methylpiperidin-1-yl)pyrimidin-4-yl)amino)-4-isopropyl-2,7-naphthyridin-1-yl)-1,3,4-oxadiazol-2-yl)propan-2-yl acetate as a yellow solid (260 mg, 86%).

Part 2. K₂CO₃ (117 mg, 0.847 mmol) was added to a solution of the compound of Part 1 (240 mg, 0.425 mmol) in MeOH (3 mL) at 0° C. and the resulting mixture stirred at rt for 1 h. The reaction mixture was diluted with EtOAc (100 mL) and washed with brine (2×50 mL). The combined organics were dried (Na₂SO₄) and evaporated to dryness in vacuo. The residue was purified by prep-HPLC-1 (Gradient (% organic): 20-45%) to afford the title compound as a white solid (44 mg, 20%). LCMS m/z=523 [M+H]⁺¹HNMR (400 MHz, DMSO-d₆) δ:10.51 (s, 1H), 10.29 (d, 1H), 8.82 (s, 1H), 8.74 (s, 1H), 8.09 (d, 1H), 6.51 (d, 1H), 6.09 (s, 1H), 5.08 (d, 1H), 4.81-4.65 (m, 2H), 3.64 (d, 1H), 3.64-3.51 (m, 1H), 3.21 (s, 2H), 1.78 (s, 2H), 1.67 (s, 6H), 1.46 (dd, 6H), 1.39 (d, 3H).

### Example 28

### 1-(6-((2-((3S,4R)-3-fluoro-4-hydroxy-3-methylpiperidin-1-yl)pyrimidin-4-yl)amino)-4-isopropyl-2,7-naphthyridin-1-yl)-N,N-dimethylazetidine-3-carboxamide

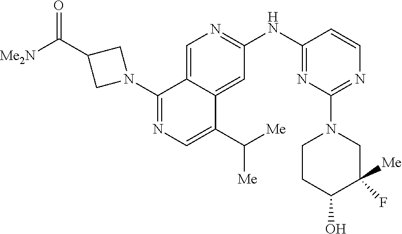 [SMILES: CC(C)c1cnc(N2CC(C(=O)N(C)C)C2)c2cnc(Nc3ccnc(N4CC[C@@H](O)[C@@](C)(F)C4)n3)cc12]

A mixture of 1-(6-chloro-4-isopropyl-2,7-naphthyridin-1-yl)-N,N-dimethylazetidine-3-carboxamide (Preparation 160, 120 mg, 0.360 mmol), (3S,4R)-1-(4-aminopyrimidin-2-yl)-3-fluoro-3-methylpiperidin-4-ol (Preparation 8, 81.4 mg, 0.360 mmol), BrettPhos Pd G3 (32.6 mg, 0.036 mmol) and Cs₂CO₃ (234 mg, 0.720 mmol) in dioxane (5 mL) was stirred at 100° C. for 2 h. The reaction was evaporated to dryness and the residue purified by prep-HPLC-1 (Gradient (% organic): 31-51%) to afford the title compound as a white solid (64 mg, 34%). LCMS m/z=523 [M+H]⁺; ¹H NMR (300 MHz, DMSO-d₆) δ: 10.09 (s, 1H), 9.05 (s, 1H), 8.49 (s, 1H), 8.04 (d, 1H), 7.98 (s, 1H), 6.52 (d, 1H), 5.05 (d, 1H), 4.82-4.62 (m, 2H), 4.56 (d, 2H), 4.44 (t, 2H), 3.92 (d, 1H), 3.68-3.46 (m, 1H), 3.22-3.04 (m, 2H), 2.94 (s, 3H), 2.87 (s, 3H), 1.82-1.70 (m, 2H), 1.44-1.27 (m, 9H).

### Example 29

### (3S,4R)-3-fluoro-1-(4-((5-isopropyl-8-((S)-2-(methoxymethyl)azetidin-1-yl)-2,7-naphthyridin-3-yl)amino)pyrimidin-2-yl)-3-methylpiperidin-4-ol

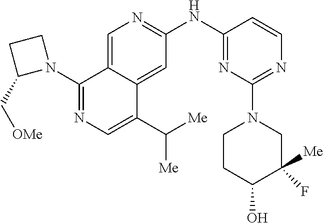 [SMILES: COC[C@@H]1CCN1c1ncc(C(C)C)c2cc(Nc3ccnc(N4CC[C@@H](O)[C@@](C)(F)C4)n3)ncc12]

(S)-6-chloro-4-isopropyl-1-(2-(methoxymethyl)azetidin-1-yl)-2,7-naphthyridine (Preparation 230, 61 mg, 0.200 mmol), (3S,4R)-1-(4-aminopyrimidin-2-yl)-3-fluoro-3-methylpiperidin-4-ol (Preparation 8, 49.7 mg, 0.220 mmol), BrettPhos Pd G3 (16.1 mg, 0.020 mmol), Cs₂CO₃ (97.8 mg, 0.300 mmol) were dissolved in 5 mL of dioxane and the mixture was stirred at 100° C. for 4 h. The reaction was evaporated to dryness and the residue purified by column chromatography (10:1, DCM/MeOH) and then further purified by prep-HPLC-21 (Gradient (% organic): 31% B to 51%) to afford the title compound as a white solid (31.6 mg, 31.9%). LCMS m/z=496 [M+H]⁺; ¹H NMR (400 MHz, DMSO-d₆) δ: 10.06 (s, 1H), 9.04 (s, 1H), 8.47 (s, 1H), 8.12-7.98 (m, 2H), 6.50 (d, 1H), 5.03 (d, 1H), 4.91-4.53 (m, 4H), 4.15 (q, 1H), 3.68 (d, 2H), 3.55 (dt, 1H), 3.23-3.02 (m, 4H), 1.77-1.67 (m, 2H), 1.46-1.23 (m, 9H).

### Example 30

### (3S,4R)-3-fluoro-1-(4-((8-((2R,3S)-3-hydroxy-2-methylazetidin-1-yl)-5-isopropyl-2,7-naphthyridin-3-yl)amino)pyrimidin-2-yl)-3-methylpiperidin-4-ol

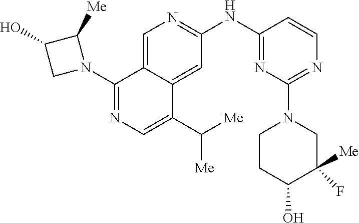 [SMILES: CC(C)c1cnc(N2C[C@H](O)[C@H]2C)c2cnc(Nc3ccnc(N4CC[C@@H](O)[C@@](C)(F)C4)n3)cc12]

A mixture of (2R,3S)-1-(6-chloro-4-isopropyl-2,7-naphthyridin-1-yl)-2-methylazetidin-3-ol (Preparation 213, 225 mg, 0.771 mmol), (3S,4R)-1-(4-aminopyrimidin-2-yl)-3-fluoro-3-methylpiperidin-4-ol (Preparation 8, 174 mg, 0.771 mmol), XPhos Pd G4 (33 mg, 0.039 mmol) and Cs₂CO₃ (503 mg, 1.542 mmol) in dioxane (5 mL) was heated to 90° C. for 2 h. The reaction mixture was diluted with 5% MeOH/DCM and washed with H₂O. The combined organics were dried (Na₂SO₄) and evaporated to dryness in vacuo. The residue was purified by ISCO chromatography (0-10% MeOH/DCM) followed by RP-ISCO (0-30% MeCN/H₂O (+0.1% TFA). The residue was treated with NaHCO₃ and extracted with 10% MeOH/DCM (3×20 mL) to afford the title compound as a white solid (157.2 mg, 42%). LCMS m/z=482 [M+H]⁺; ¹H NMR (300 MHz, DMSO-d₆) δ: 10.04 (1H, s), 9.04 (1H, s), 8.46 (1H, s), 8.02-7.99 (2H, m), 6.49 (1H, d), 5.57 (1H, d), 5.01 (1H, d), 4.84 (1H, t), 4.74-4.60 (2H, m), 4.40-4.36 (1H, m), 4.18-4.12 (1H, m), 3.74 (1H, t), 3.60-3.48 (1H, m), 3.18-3.08 (2H, m), 1.74-1.69 (2H, m), 1.41-1.29 (12H, m).

### Example 31

### (3S,4R)-3-fluoro-1-(4-((8-((2R,3R)-3-hydroxy-2-methylazetidin-1-yl)-5-isopropyl-2,7-naphthyridin-3-yl)amino)pyrimidin-2-yl)-3-methylpiperidin-4-ol or (3S,4R)-3-fluoro-1-(4-((8-((2S,3S)-3-hydroxy-2-methylazetidin-1-yl)-5-isopropyl-2,7-naphthyridin-3-yl)amino)pyrimidin-2-yl)-3-methylpiperidin-4-ol

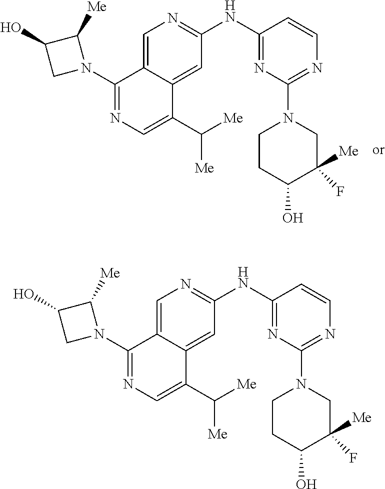 [SMILES: CC(C)c1cnc(N2C[C@@H](O)[C@H]2C)c2cnc(Nc3ccnc(N4CC[C@@H](O)[C@@](C)(F)C4)n3)cc12 ; "or" ; CC(C)c1cnc(N2C[C@H](O)[C@@H]2C)c2cnc(Nc3ccnc(N4CC[C@@H](O)[C@@](C)(F)C4)n3)cc12]

A mixture of (2R,3R)-1-(6-chloro-4-isopropyl-2,7-naphthyridin-1-yl)-2-methylazetidin-3-ol or (2S,3S)-1-(6-chloro-4-isopropyl-2,7-naphthyridin-1-yl)-2-methylazetidin-3-ol (Peak 4 from Preparation 213A, 214, 215, 216, 80 mg, 0.274 mmol), (3S,4R)-1-(4-aminopyrimidin-2-yl)-3-fluoro-3-methylpiperidin-4-ol (Preparation 8, 61.9 mg, 0.274 mmol), BrettPhos Pd G3 (24.8 mg, 0.027 mmol) and Cs₂CO₃ (503 mg, 1.542 mmol) in dioxane (5 mL) was heated to 100° C. for 3 h. The reaction mixture was diluted with H₂O (100 mL) and extracted with EtOAc. The combined organics were washed with brine, dried (Na₂SO₄) and evaporated to dryness in vacuo. The residue was purified by column chromatography (10:1 DCM:MeOH) and further purified by Prep-HPLC-18 (Gradient (% organic) 24-39%) to afford the title compound as a pale yellow solid (43.6 mg, 33%). LCMS m/z=482 [M+H]*; ¹H NMR (300 MHz, DMSO-d₆) δ:10.04 (s, 1H), 8.98 (s, 1H), 8.44 (s, 1H), 8.01 (d, 1H), 7.95 (s, 1H), 6.47 (d, 1H), 5.52 (d, 1H), 5.04 (d, 1H), 4.86 (t, 1H), 4.79-4.53 (m, 3H), 4.32 (d, 2H), 3.52 (dd, 1H), 3.33-3.01 (m, 3H), 1.72 (s, 2H), 1.44-1.20 (m, 12H).

### Example 32

### (3S,4R)-3-fluoro-1-(4-((5-isopropyl-8-((S)-2-methylazetidin-1-yl)-2,7-naphthyridin-3-yl)amino)pyrimidin-2-yl)-3-methylpiperidin-4-ol or (3S,4R)-3-fluoro-1-(4-((5-isopropyl-8-((R)-2-methylazetidin-1-yl)-2,7-naphthyridin-3-yl)amino)pyrimidin-2-yl)-3-methylpiperidin-4-ol

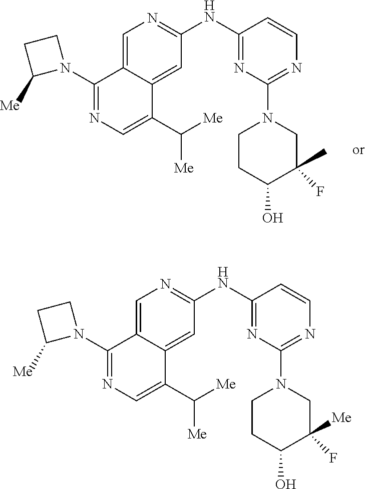 [SMILES: CC(C)c1cnc(N2CC[C@@H]2C)c2cnc(Nc3ccnc(N4CC[C@@H](O)[C@@](C)(F)C4)n3)cc12 ; "or" ; CC(C)c1cnc(N2CC[C@H]2C)c2cnc(Nc3ccnc(N4CC[C@@H](O)[C@@](C)(F)C4)n3)cc12]

Part 1. A mixture of 6-chloro-4-isopropyl-1-(2-methylazetidin-1-yl)-2,7-naphthyridine (Preparation 170, 80 mg, 0.290 mmol), (3S,4R)-1-(4-aminopyrimidin-2-yl)-3-fluoro-3-methylpiperidin-4-ol (Preparation 8, 65.4 mg, 0.290 mmol), Cs₂CO₃ (187.2 mg, 0.579 mmol), and XantPhos Pd G2 (77.2 mg, 0.087 mmol) in dioxane (15 mL) was stirred at 100° C. for 16 h. The reaction mixture was evaporated to dryness and the residue purified by prep-TLC (10:1 DCM/MeOH) to afford (3S,4R)-3-fluoro-1-(4-((5-isopropyl-8-(2-methylazetidin-1-yl)-2,7-naphthyridin-3-yl)amino)pyrimidin-2-yl)-3-methylpiperidin-4-ol or (3R,4S)-3-fluoro-1-(4-((5-isopropyl-8-(2-methylazetidin-1-yl)-2,7-naphthyridin-3-yl)amino)pyrimidin-2-yl)-3-methylpiperidin-4-ol as a yellow solid (100 mg, 99%). LCMS m/z=465 [M+H]⁺

Part 2. The racemate from Part 1 was purified by chiral-HPLC (CHIRALPAK IA, 20×250 mm, 5 mm; 30% EtOH/Hex (+8 mmol/L NH₃/MeOH) to afford (3S,4R)-3-fluoro-1-(4-((5-isopropyl-8-((S)-2-methylazetidin-1-yl)-2,7-naphthyridin-3-yl)amino)pyrimidin-2-yl)-3-methylpiperidin-4-ol or (3R,4S)-3-fluoro-1-(4-((5-isopropyl-8-((S)-2-methylazetidin-1-yl)-2,7-naphthyridin-3-yl)amino)pyrimidin-2-yl)-3-methylpiperidin-4-ol and (3S,4R)-3-fluoro-1-(4-((5-isopropyl-8-((R)-2-methylazetidin-1-yl)-2,7-naphthyridin-3-yl)amino)pyrimidin-2-yl)-3-methylpiperidin-4-ol or (3R,4S)-3-fluoro-1-(4-((5-isopropyl-8-((R)-2-methylazetidin-1-yl)-2,7-naphthyridin-3-yl)amino)pyrimidin-2-yl)-3-methylpiperidin-4-ol as pale-yellow solids.

Peak 1: LCMS m/z=465 [M+H]*; ¹H NMR (300 MHz, DMSO-d₆) δ: 10.04 (s, 1H), 9.01 (s, 1H), 8.45 (s, 1H), 8.05-7.94 (m, 2H), 6.48 (d, 1H), 5.02 (d, 1H), 4.74 (d, 4H), 4.08 (d, 1H), 3.48 (s, 1H), 3.31 (s, 1H), 3.16 (s, 2H), 2.48 (d, 1H), 2.05 (s, 1H), 1.71 (s, 2H), 1.45-1.35 (m, 3H), 1.34-1.25 (m, 9H).

Peak 2: LCMS m/z=465 [M+H]*; ¹H NMR (400 MHz, DMSO-d₆) δ: 10.06 (s, 1H), 9.04 (s, 1H), 8.47 (s, 1H), 8.07-7.97 (m, 2H), 6.51 (d, 1H), 5.05 (d, 1H), 4.83-4.73 (m, 2H), 4.69-4.62 (m, 2H), 4.12 (q, 1H), 3.23-3.11 (m, 4H), 2.51 (s, 2H), 1.47-1.38 (m, 3H), 1.37-1.29 (m, 9H).

### Example 33 and 34

### (3S,4R)-3-fluoro-1-(4-((8-((2R,3R)-3-fluoro-2-methylazetidin-1-yl)-5-isopropyl-2,7-naphthyridin-3-yl)amino)pyrimidin-2-yl)-3-methylpiperidin-4-ol or (3S,4R)-3-fluoro-1-(4-((8-((2S,3S)-3-fluoro-2-methylazetidin-1-yl)-5-isopropyl-2,7-naphthyridin-3-yl)amino)pyrimidin-2-yl)-3-methylpiperidin-4-ol and (3S,4R)-3-fluoro-1-(4-((8-((2S,3R)-3-fluoro-2-methylazetidin-1-yl)-5-isopropyl-2,7-naphthyridin-3-yl)amino)pyrimidin-2-yl)-3-methylpiperidin-4-ol or (3S,4R)-3-fluoro-1-(4-((8-((2R,3S)-3-fluoro-2-methylazetidin-1-yl)-5-isopropyl-2,7-naphthyridin-3-yl)amino)pyrimidin-2-yl)-3-methylpiperidin-4-ol

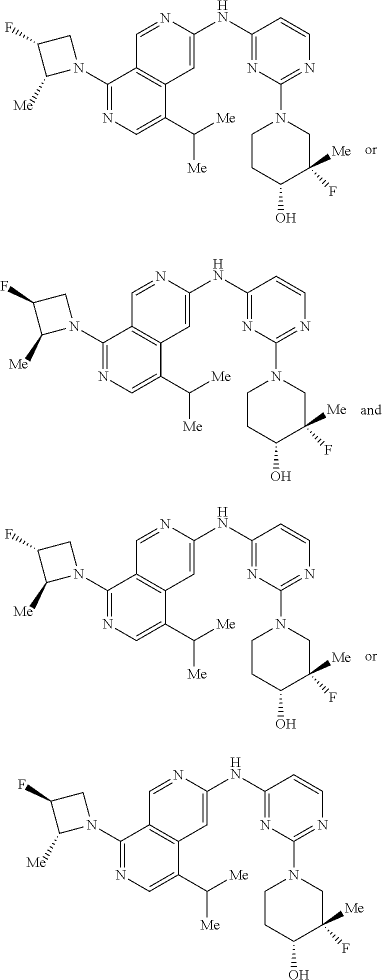

Part 1. A mixture of 6-chloro-1-(3-fluoro-2-methylazetidin-1-yl)-4-isopropyl-2,7-naphthyridine (Preparation 248, 250 rig, 0.851 mmol), (3S,4R)-1-(4-aminopyrimidin-2-yl)-3-fluoro-3-methylpiperidin-4-ol (Preparation 8, 192 mg, 0.851 mmol), Cs₂CO₃ (830 mg, 2.55 mmol), and RuPhos Pd G3 (77 mg, 0.085 mmol) in dioxane (10 mL) was stirred at 100° C. under N₂ for 3 h. The reaction was quenched with H₂O and extracted with EtOAc. The combined extracts were evaporated to dryness and the residue purified by prep-HPLC-18 to afford (3S,4R)-3-fluoro-1-(4-((8-(3-fluoro-2-methylazetidin-1-yl)-5-isopropyl-2,7-naphthyridin-3-yl)amino)pyrimidin-2-yl)-3-methylpiperidin-4-ol as a white solid (120 mg) Part 2. The compound of Part 1 was purified by chiral-HPLC (CHIRALPAK 1E, 20×250 mm. 5 mm: 5% EtOH/MTB (10 mM NH₃/MEOH)) to afford the title compounds as white solids.

Peak 1: (3S,4R)-3-fluoro-1-(4-((8-((2R,3R)-3-fluoro-2-methylazetidin-1-yl)-5-isopropyl-2,7-naphthyridin-3-yl)amino)pyrimidin-2-yl)-3-methylpiperidin-4-ol or (3S,4R)-3-fluoro-1-(4-((8-((2S,3S)-3-fluoro-2-methylazetidin-1-yl)-5-isopropyl-2,7-naphthyridin-3-yl)amino)pyrimidin-2-yl)-3-methylpiperidin-4-ol; LCMS m/z=484 [M+H]⁺; ¹H NMR (300 MHz, MeOH-d₄) δ: 9.10 (s, 1H), 8.57 (s, 1H), 8.05-7.96 (m, 1H), 7.94 (s, 1H), 6.46 (d, 1H), 5.05 (s, 1H), 4.71 (d, 2H), 4.15 (s, 1H), 3.70 (ddd, 1H), 3.47 (s, 1H), 3.24 (d, 1H), 1.97-1.84 (m, 2H), 1.56-1.48 (m, 5H), 1.47-1.37 (m, 8H), 1.30 (s, 2H).

Peak 3: (3S,4R)-3-fluoro-1-(4-((8-(2S,3R)-3-fluoro-2-ethylazetidin-1-yl)-isopropyl-2,7-naphthyridin-3-yl)amino)pyrimidin-2-yl)-3-methylpiperidin-4-ol or (3S,4R)-3-fluoro-1-(4-((8-((2R,3S)-3-fluoro-2-methylazetidin-1-yl)-5-isopropyl-2,7-napthridin-3-yl)amino)pyrimidin-2-yl-3-methylpiperidin-4-ol; LCMS m/z=484 [M+H]⁺; ¹H NMR (400 MHz, DMSO-d₆) δ: 10.29 (s, 1H), 9.06 (s, 1H), 8.49 (s, 1H), 8.06 (d, 1H), 7.91 (s, 1H), 6.56 (d, 1H), 5.47 (d, 1H), 5.25-4.33 (m, 5H), 3.69-3.48 (m, 1H), 3.25-2.86 (m, 2H), 1.98-1.57 (m, 2H), 1.57-0.99 (m, 13H).

### Example 35

### 6-(3-((2-((3S,4R)-3-fluoro-4-hydroxy-3-methylpiperidin-1-yl)pyrimidin-4-yl)amino)-5-isopropylisoquinolin-8-yl)-1-thia-6-azaspiro[3.3]heptane 1,1-dioxide

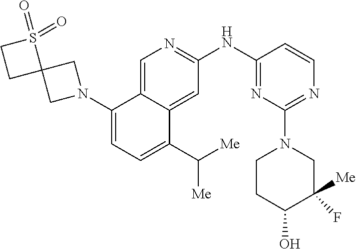 [SMILES: CC(C)c1ccc(N2CC3(CCS3(=O)=O)C2)c2cnc(Nc3ccnc(N4CC[C@@H](O)[C@@](C)(F)C4)n3)cc12]

Cs₂CO₃ (64.8 mg, 0.199 mmol) was added to a solution of 6-(3-chloro-5-isopropylisoquinolin-8-yl)-1-thia-6-azaspiro[3.3]heptane 1,1-dioxide (Preparation 126, 35 mg. 0,100 mmol) and (3S,4R)-1-(4-aminopyrimidin-2-yl)-3-fluoro-3-methylpiperidine-4-ol (Preparation 8, 22.5 mug, 0.100 mmol) in dry dioxane. CPhos (4.34 mg, 0.01 μmol) and Pd₂(dba)₃·CHCl₃ (1.46 mg, 1.42 μmol) was added under N₂ the reaction stirred at 100° C. for 2 h. The solvent was removed by evaporation in vacuo and the residue purified by HPLC-23 (Gradient (% organic) 35-55%) to afford the title compound as a pale yellow solid (27 mg). LCMS m/z=541 [M+H]⁺; ¹H NMR (400 MHz, DMSO-d₆) δ: 9.95 (s, 1H), 9.02 (s, 1H), 8.66 (s, 1H), 8.00 (d, 1H), 7.46 (d, 1H), 6.56 (d, 1H), 6.46 (d, 1H), 5.04 (d, 1H), 4.84-4.64 (m, 2H), 4.62 (d, 2H), 4.37 (dd, 2H), 4.20-4.06 (m, 2H), 3.67-3.45 (m, 2H), 3.24-3.00 (m, 2H), 2.46-2.38 (m, 2H), 1.83-1.68 (m, 2H), 1.43-1.21 (m, 9H).

### Example 36 and 37

### (R)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile and (S)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile

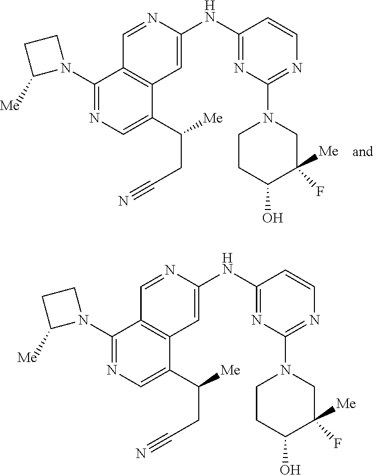 [SMILES: C[C@H](CC#N)c1cnc(N2CC[C@H]2C)c2cnc(Nc3ccnc(N4CC[C@@H](O)[C@@](C)(F)C4)n3)cc12 ; "and" ; C[C@@H]1CCN1c1ncc([C@@H](C)CC#N)c2cc(Nc3ccnc(N4CC[C@@H](O)[C@@](C)(F)C4)n3)ncc12]

The title compounds were prepared from 3-(6-chloro-1-((R)-2-methylazetidin-1-yl)-2,7-naphthyridin-4-yl)butanenitrile (Preparation 329) and (3S,4R)-1-(4-aminopyrimidin-2-yl)-3-fluoro-3-methylpiperidin-4-ol (Preparation 8) using an analogous 2-part procedure as described for Example 1. Chiral-HPLC (CHIRALPAK ADH, 20×250 mm, 5 mm; 50% IPA/Hex (8 mmol/L NH₃/MeOH)) to afford the title compounds as white solids.

Peak 1 (Example 36). (R)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile or (S)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile LCMS m/z=491 [M+H]⁺; ¹H NMR (300 MHz, DMSO-d₆) δ: 10.04 (s, 1H), 9.03 (s, 1H), 8.32 (s, 1H), 8.03 (d, 2H), 6.56 (d, 1H), 5.01 (d, 1H), 4.85-4.52 (m, 4H), 4.17 (q, 1H), 3.62-3.39 (m, 2H), 3.23-2.79 (m, 5H), 2.10-1.94 (m, 1H), 1.88-1.68 (m, 2H), 1.48-1.25 (m, 9H).

Peak 2 (Example 37). (R)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile or (S)-3-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)butanenitrile. LCMS m/z=491 [M+H]⁺; ¹H NMR (300 MHz, DMSO-d₆) δ: 10.05 (s, 1H), 9.03 (s, 1H), 8.31 (s, 1H), 8.12-7.89 (m, 2H), 6.56 (d, 1H), 5.01 (s, 1H), 4.91-4.49 (m, 4H), 4.15 (q, 1H), 3.65-3.39 (m, 2H), 3.23-2.81 (m, 5H), 2.16-1.99 (m, 1H), 1.88-1.59 (m, 2H), 1.61-1.30 (m, 9H).

### Example 38

### (3S,4R)-1-(4-((8-(3-(1H-1,2,3-triazol-1-yl)azetidin-1-yl)-5-isopropylisoquinolin-3-yl)amino)pyrimidin-2-yl)-3-fluoro-3-methylpiperidin-4-ol

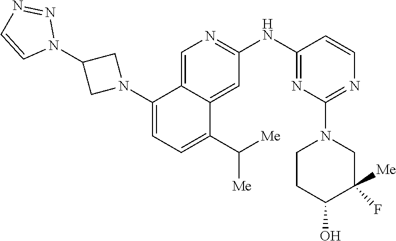 [SMILES: CC(C)c1ccc(N2CC(n3ccnn3)C2)c2cnc(Nc3ccnc(N4CC[C@@H](O)[C@@](C)(F)C4)n3)cc12]

A mixture of 8-(3-(1H-1,2,3-triazol-1-yl)azetidin-1-yl)-3-chloro-5-isopropylisoquinoline (Preparation 122, 130 mg, 0.396 mmol), (3S,4R)-1-(4-aminopyrimidin-2-yl)-3-fluoro-3-methylpiperidin-4-ol (Preparation 8, 89.5 mg, 0.396 mmol), Cs₂CO₃ (258 mg, 0.792 mmol), RuPhos Pd G3 (33.1 mg, 0.034 mmol) in dioxane (15 mL) was stirred at 100° C. under N₂ for 16 h. The solids were removed by filtration and the filtrate evaporated to dryness in vacuo. The residue was purified by prep-TLC (20:1 DCM/MeOH) and the residue further purified by prep-HPLC-21 (Gradient (% organic) 35-45%) to give the title compound as a yellow solid (62.1 mg, 99%). LCMS m/z=517 [M+H]⁺; ¹H NMR (300 MHz, DMSO-d₆) δ: 9.88 (s, 1H), 9.12 (s, 1H), 8.64 (s, 1H), 8.49 (d, 1H), 8.00 (d, 1H), 7.84 (d, 1H), 7.47 (d, 1H), 6.56 (d, 1H), 6.48 (d, 1H), 5.72 (q, 1H), 5.03 (d, 1H), 4.83-4.65 (m, 4H), 4.50 (t, 2H), 3.67-3.46 (m, 2H), 3.26-3.05 (m, 2H), 1.75 (s, 2H), 1.37-1.28 (m, 9H).

### Example 39

### 8-((S)-2-(aminomethyl)azetidin-1-yl)-N-(2-((3R,4S)-3-fluoro-4-methoxypiperidin-1-yl)pyrimidin-4-yl)-5-isopropylisoquinolin-3-amine

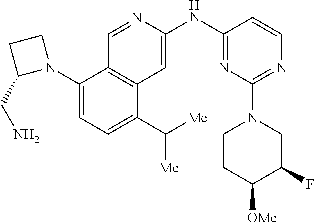 [SMILES: CO[C@H]1CCN(c2nccc(Nc3cc4c(C(C)C)ccc(N5CC[C@H]5CN)c4cn3)n2)C[C@H]1F]

Part 1. A mixture of 2-((3R,4S)-3-fluoro-4-methoxypiperidin-1-yl)pyrimidin-4-amine (Preparation 32, 40.6 mg, 0.180 mmol), tert-butyl (S)-((1-(3-chloro-5-isopropylisoquinolin-8-yl)azetidin-2-yl)methyl)carbamate (Preparation 120, 70 mg, 0.180 mmol), Cs₂CO₃ (117 mg, 0.360 mmol) and RuPhos Pd G3 (30 mg, 0.036 mmol) in dioxane (10 mL) was heated under N₂ at 100° C. for 16 h. The solids were removed by filtration and the filtrate evaporated to dryness in vacuo and the residue purified by prep-TLC (EtOAc) to afford tert-butyl (((S)-1-(3-((2-((3R,4S)-3-fluoro-4-methoxypiperidin-1-yl)pyrimidin-4-yl)amino)-5-isopropylisoquinolin-8-yl)azetidin-2-yl)methyl)carbamate as a pale yellow solid.

Part 2. TFA (2 mL) was added to a solution of tert-butyl (((S)-1-(3-((2-((3R,4S)-3-fluoro-4-methoxypiperidin-1-yl)pyrimidin-4-yl)amino)-5-isopropylisoquinolin-8-yl)azetidin-2-yl)methyl)carbamate in DCM (6 mL) at 0° C. and the resulting mixture stirred for 3 h. The reaction mixture was evaporated to dryness and the residue purified by prep-HPLC-23 to afford the title compound as a yellow solid. LCMS m/z=479 [M+H]⁺; ¹H NMR (300 MHz, DMSO-d₆) δ: 9.14 (d, 1H), 8.68 (d, 1H), 7.98 (dd, 1H), 7.49 (d, 1H), 6.81-6.66 (m, 1H), 6.38 (dd, 1H), 4.81 (s, 1H), 4.66 (s, 1H), 4.55-4.45 (m, 1H), 4.45 (s, 1H), 4.40 (s, 1H), 3.80 (q, 1H), 3.72 (d, 2H), 3.68-3.54 (m, 1H), 3.50 (s, 3H), 3.47-3.37 (m, 1H), 3.31-3.08 (m, 2H), 2.48 (d, 1H), 2.44 (s, 1H), 1.96-1.86 (m, 2H), 1.43-1.34 (m, 6H).

### Example 40

### (3R,4S)-1-(4-((8-(3-((1H-1,2,3-triazol-1-yl)methyl)azetidin-1-yl)-5-isopropylisoquinolin-3-yl)amino)pyrimidin-2-yl)-3-fluoro-4-methylpiperidin-4-ol

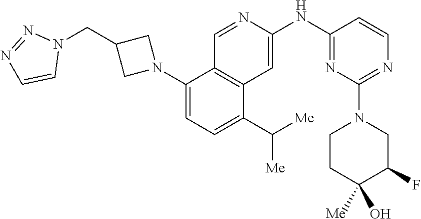 [SMILES: CC(C)c1ccc(N2CC(Cn3ccnn3)C2)c2cnc(Nc3ccnc(N4CC[C@](C)(O)[C@H](F)C4)n3)cc12]

A solution of (3R,4S)-1-(4-aminopyrimidin-2-yl)-3-fluoro-4-methylpiperidin-4-ol (Preparation 14, 19.74 mg, 0.087 mmol), 8-(3-((1H-1,2,3-triazol-1-yl)methyl)azetidin-1-yl)-3-chloro-5-isopropylisoquinoline (Preparation 127, 30 mg, 0.087 mmol), BrettPhos Pd G4 (6.02 mg, 6.54 μmol) and Cs₂CO₃ (85 mg, 0.262 mmol) in Dioxane (0.9 ml) was purged with N₂ for 5 min before heating at 90° C. for a 3 h. The reaction was diluted with DCM (+10% MeOH) and filtered through celite. The filtrate was evaporated to dryness in vacuo and the residue purified using RP-ISCO chromatography (0-60% H₂O/MeCN (+0.1% TFA). The residue was further purified by dissolving in DCM (+10% MeOH) washing with sat. NaHCO₃ solution. The aqueous layer was washed 5× with DCM (w/10% MeOH). The combined organics were dried (Na₂SO₄) and evaporated to dryness in vacuo to afford the title compound as a yellow solid (16.7 mg, 36%). LCMS m/z=479 [M+H]*; ¹H NMR (400 MHz, DMSO-d₆) δ: 9.88 (s, 1H), 9.06 (s, 1H), 8.63 (s, 1H), 8.24 (d, 1H), 8.01 (d, 1H), 7.76 (d, 1H), 7.41 (d, 1H), 6.50 (d, 1H), 6.41 (d, 1H), 4.82 (s, 1H), 4.77 (d, 2H), 4.49-4.36 (m, 1H), 4.27 (s, 1H), 4.25 (s, 1H), 4.20 (d, 2H), 3.99 (dd, 2H), 3.62 (ddd, 1H), 3.50 (q, 3H), 3.25 (q, 1H), 1.73 (s, 1H), 1.62-1.51 (m, 1H), 1.33-1.22 (m, 10H).

### Example 41

### N-(2-((3S,4R)-3-fluoro-4-methoxypiperidin-1-yl)pyrimidin-4-yl)-5-isopropyl-8-(6-methyl-1,6-diazaspiro[3.3]heptan-1-yl)-2,7-naphthyridin-3-amine

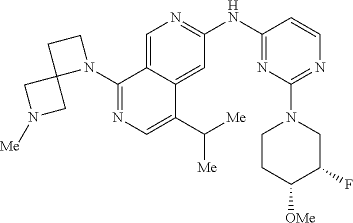 [SMILES: CO[C@@H]1CCN(c2nccc(Nc3cc4c(C(C)C)cnc(N5CCC56CN(C)C6)c4cn3)n2)C[C@@H]1F]

Part 1. A mixture of tert-butyl 1-(6-chloro-4-isopropyl-2,7-naphthyridin-1-yl)-1,6-diazaspiro[3.3]heptane-6-carboxylate (Preparation 158, 100 mg, 0.248 mmol), 2-((3S,4R)-3-fluoro-4-methoxypiperidin-1-yl)pyrimidin-4-amine (Preparation 33, 61.8 mg, 0.272 mmol), BrettPhos Pd G3 (22.5 mg, 0.025 mmol) and Cs₂CO₃ (127.4 mg, 0.5 mmol) in dioxane was heated together at 100° C. for 2 h under microwave conditions. The solids were removed by filtration and the filtrate evaporated to dryness in vacuo. The residue was purified by prep-TLC (DCM/MeOH, 10:1) to afford tert-butyl 1-(6-((2-((3S,4R)-3-fluoro-4-methoxypiperidin-1-yl)pyrimidin-4-yl)amino)-4-isopropyl-2,7-naphthyridin-1-yl)-1,6-diazaspiro[3.3]heptane-6-carboxylate as a white solid (120 mg, 82%). LCMS m/z=593 [M+H]⁺

Part 2. TFA (1 mL) was added to a solution of tert-butyl 1-(6-((2-((3S,4R)-3-fluoro-4-methoxypiperidin-1-yl)pyrimidin-4-yl)amino)-4-isopropyl-2,7-naphthyridin-1-yl)-1,6-diazaspiro[3.3]heptane-6-carboxylate as a white solid (Part 1, 120 mg, 0.203 mmol) in DCM (3 mL) and the mixture stirred at rt for 2 h. The reaction mixture was evaporated to dryness to afford N-(2-((3S,4R)-3-fluoro-4-methoxypiperidin-1-yl)pyrimidin-4-yl)-5-isopropyl-8-(1,6-diazaspiro[3.3]heptan-1-yl)-2,7-naphthyridin-3-amine as a colourless oil (100 mg). LCMS m/z=593 [M+H]⁺

Part 3. NaBH₃CN (20.4 mg, 0.325 mmol) was added to a solution of N-(2-((3S,4R)-3-fluoro-4-methoxypiperidin-1-yl)pyrimidin-4-yl)-5-isopropyl-8-(1,6-diazaspiro[3.3]heptan-1-yl)-2,7-naphthyridin-3-amine (Part 2, 80 mg, 0.162 mmol), HCHO (28.1 mg, 0.325 mmol) and AcOH (cat) in MeOH (5 mL) and the mixture stirred at rt overnight. The reaction mixture was evaporated to dryness in vacuo and the residue purified by prep-HPLC-5 (Gradient (% organic): 12-20%) to afford the title compound as a yellow solid (6.1 mg, 7%). LCMS m/z=507 [M+H]⁺; ¹H NMR (300 MHz, MeOH-d₄) δ: 9.12 (s, 1H), 8.65-8.36 (m, 2H), 8.10 (s, 1H), 8.00 (d, 1H), 6.40 (d, 1H), 5.05-4.92 (m, 2H), 4.86-4.80 (m, 1H), 4.71-4.52 (m, 3H), 4.46-4.21 (m, 3H), 3.74-3.56 (m, 2H), 3.49 (s, 3H), 3.41 (td, 2H), 2.72 (t, 2H), 1.95-1.85 (m, 2H), 1.39 (t, 6H).

### Example 42

### N-(2-((3R,4S)-3-fluoro-4-methoxypiperidin-1-yl)pyrimidin-4-yl)-5-isopropyl-8-(6-methyl-1,6-diazaspiro[3.3]heptan-1-yl)-2,7-naphthyridin-3-amine

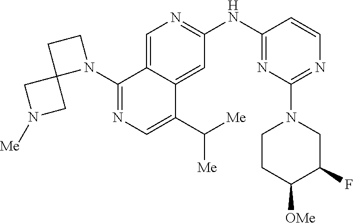 [SMILES: CO[C@H]1CCN(c2nccc(Nc3cc4c(C(C)C)cnc(N5CCC56CN(C)C6)c4cn3)n2)C[C@H]1F]

The title compound was prepared from tert-butyl 1-(6-chloro-4-isopropyl-2,7-naphthyridin-1-yl)-1,6-diazaspiro[3.3]heptane-6-carboxylate (Preparation 158) and 2-((3R,4S)-3-fluoro-4-methoxypiperidin-1-yl)pyrimidin-4-amine (Preparation 32) using an analogous method to that described for Example 41 (150051). LCMS m/z=507 [M+H]¹; ¹H NMR (300 MHz, MeOH-d₄) δ: 9.16 (s, 1H), 8.58 (s, 1H), 8.12 (s, 1H), 8.01 (d, 1H), 6.42 (d, 1H), 5.05-4.87 (m, 2H), 4.64 (q, 4H), 4.44-4.24 (m, 3H), 3.78-3.53 (m, 2H), 3.49 (s, 3H), 3.43-3.33 (m, 2H), 3.13-2.91 (m, 3H), 2.73 (t, 2H), 1.96-1.73 (m, 2H), 1.40 (dd, 6H).

### Example 43

### N-(2-((3R,4S)-3-fluoro-4-methoxy-3-methylpiperidin-1-yl)pyrimidin-4-yl)-5-isopropyl-8-(6-methyl-1,6-diazaspiro[3.3]heptan-1-yl)-2,7-naphthyridin-3-amine or N-(2-((3S,4R)-3-fluoro-4-methoxy-3-methylpiperidin-1-yl)pyrimidin-4-yl)-5-isopropyl-8-(6-methyl-1,6-diazaspiro[3.3]heptan-1-yl)-2,7-naphthyridin-3-amine

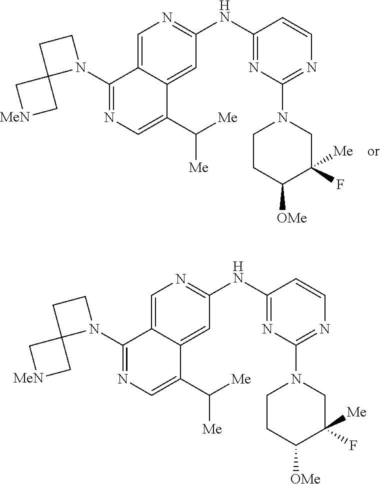 [SMILES: CO[C@H]1CCN(c2nccc(Nc3cc4c(C(C)C)cnc(N5CCC56CN(C)C6)c4cn3)n2)C[C@@]1(C)F ; CO[C@@H]1CCN(c2nccc(Nc3cc4c(C(C)C)cnc(N5CCC56CN(C)C6)c4cn3)n2)C[C@]1(C)F]

The title compound was prepared from tert-butyl 1-(6-chloro-4-isopropyl-2,7-naphthyridin-1-yl)-1,6-diazaspiro[3.3]heptane-6-carboxylate (Preparation 158) and 2-((3S,4R)-3-fluoro-4-methoxy-3-methylpiperidin-1-yl)pyrimidin-4-amine or 2-((3R,4S)-3-fluoro-4-methoxy-3-methylpiperidin-1-yl)pyrimidin-4-amine (Peak 2, Preparation 40, 41, 42 and 43) using an analogous method to that described for Example 41 (150051). Prep-HPLC-2 (Gradient (% organic): 44-65%). LCMS m/z=521 [M+H]⁺; ¹H NMR (400 MHz, DMSO-d₆) δ: 10.06 (s, 1H), 9.15 (s, 1H), 8.41 (s, 1H), 8.15-7.90 (m, 2H), 6.54 (d, 1H), 4.77-4.60 (m, 2H), 4.55 (t, 2H), 4.45-4.26 (m, 2H), 3.60-3.36 (m, 9H), 3.26-3.10 (m, 3H), 2.64 (q, 2H), 2.03-1.89 (m, 1H), 1.70-1.57 (m, 1H), 1.40 (d, 3H), 1.33 (t, 6H).

### Example 44

### 1-(6-((2-((3R,4S)-3-fluoro-4-methoxypiperidin-1-yl)pyrimidin-4-yl)amino)-4-isopropyl-2,7-naphthyridin-1-yl)azetidin-3-ol

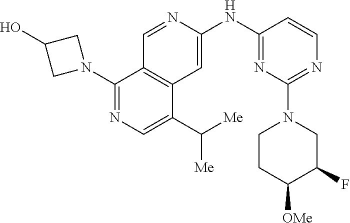 [SMILES: CO[C@H]1CCN(c2nccc(Nc3cc4c(C(C)C)cnc(N5CC(O)C5)c4cn3)n2)C[C@H]1F]

A mixture of 1-(6-chloro-4-isopropyl-2,7-naphthyridin-1-yl)azetidin-3-ol (Preparation 161, 110 mg, 0.396 mmol), 2-((3R,4S)-3-fluoro-4-methoxypiperidin-1-yl)pyrimidin-4-amine (Preparation 32, 89.5 mg, 0.396 mmol), Brettphos Pd G3 (35.8 mg, 0.040 mmol) and Cs₂CO₃ (383 mg, 1.18 mmol) in dioxane (10 mL) was heated at 100° C. for 3 h under N₂. The reaction was quenched with H₂O and extracted with EtOAc. The combined extracts were dried and evaporated to dryness in vacuo. The residue was purified by prep-HPLC-18 (Gradient (% organic) 29-39%) to afford the title compound as a white solid (24 mg, 13%). LCMS m/z=468 [M+H]*; ¹H NMR (400 MHz, DMSO-d₆) δ:10.08 (s, 1H), 9.03 (d, 1H), 8.49 (s, 1H), 8.05 (d, 1H), 7.97 (s, 1H), 6.52 (d, 1H), 5.70 (d, 1H), 5.02-4.82 (m, 1H), 4.71 (td, 1H), 4.63-4.52 (m, 3H), 4.47 (d, 1H), 4.16-4.00 (m, 2H), 3.68-3.38 (m, 1H), 3.37 (s, 3H), 3.30-3.21 (m, 2H), 1.87-1.63 (m, 2H), 1.31 (dd, 6H).

### Example 45-154

The title compounds were prepared from the appropriate chloride (RCl) and amine (Amine-1 to Amine-20) using an analogous method to that described for Example 44 using an appropriate palladium catalyst as noted in table.

Amine-1, (3S,4R)-1-(4-aminopyrimidin-2-yl)-3-fluoro-3-methylpiperidin-4-ol (Preparation 8); Amine-2, 2-(1-oxa-7-azaspiro[3.5]nonan-7-yl)pyrimidin-4-amine (Preparation 62); Amine-3, (3S,4R)-1-(4-aminopyrimidin-2-yl)-4-methoxypiperidin-3-ol or (3R,4S)-1-(4-aminopyrimidin-2-yl)-4-methoxypiperidin-3-ol (Peak 1 from Preparation 16 and 17); Amine-4, (3S,4S)-1-(4-aminopyrimidin-2-yl)-4-methoxypiperidin-3-ol or (3R,4R)-1-(4-aminopyrimidin-2-yl)-4-methoxypiperidin-3-ol (Peak 1 from Preparation 18 and 19); Amine-5, 1-(4-aminopyrimidin-2-yl)-4-methylpiperidin-4-ol (Preparation 23); Amine-6, 1-(4-aminopyrimidin-2-yl)-4-(hydroxymethyl)piperidin-4-ol (Preparation 61); Amine-7, (4S,5R)-1-(4-aminopyrimidin-2-yl)-5-fluoro-3,3-dimethylpiperidin-4-ol or (4R,5S)-1-(4-aminopyrimidin-2-yl)-5-fluoro-3,3-dimethylpiperidin-4-ol (Peak 2 from Preparation 21 and 22); Amine-8, (3S,4R,5R)-1-(4-aminopyrimidin-2-yl)-3,5-difluoro-3-methylpiperidin-4-ol or (3R,4S,5S)-1-(4-aminopyrimidin-2-yl)-3,5-difluoro-3-methylpiperidin-4-ol (Peak 1 from Preparation 48, 49, 50, 51); Amine-9, (3R,4S,5S)-1-(4-aminopyrimidin-2-yl)-3,5-difluoro-3-methylpiperidin-4-ol or (3S,4R,5R)-1-(4-aminopyrimidin-2-yl)-3,5-difluoro-3-methylpiperidin-4-ol (Peak 2 from Preparation 48, 49, 50, 51); Amine-10, (3S,4S,5S)-1-(4-aminopyrimidin-2-yl)-3,5-difluoro-3-methylpiperidin-4-ol or (3R,4R,5R)-1-(4-aminopyrimidin-2-yl)-3,5-difluoro-3-methylpiperidin-4-ol (Peak 3 from Preparation 48, 49, 50, 51); Amine-11, (3R,4R,5R)-1-(4-aminopyrimidin-2-yl)-3,5-difluoro-3-methylpiperidin-4-ol or (3S,4S,5S)-1-(4-aminopyrimidin-2-yl)-3,5-difluoro-3-methylpiperidin-4-ol (Peak 4 from Preparation 48, 49, 50, 51); Amine-12, 2-((3R,4S)-3-fluoro-4-(methoxy-d3)piperidin-1-yl)pyrimidin-4-amine (Preparation 30); Amine-13, 2-((3S,4R)-3-fluoro-4-(methoxy-d3)piperidin-1-yl)pyrimidin-4-amine (Preparation 31); Amine-14, 2-(((3R,4S)-1-(4-aminopyrimidin-2-yl)-3-fluoropiperidin-4-yl)oxy)ethan-1-ol (Preparation 27); Amine-15, 2-(((3S,4R)-1-(4-aminopyrimidin-2-yl)-3-fluoropiperidin-4-yl)oxy)ethan-1-ol (Preparation 25); Amine-16, (3R,4S)-1-(4-aminopyrimidin-2-yl)-3-fluoropiperidin-4-ol (J Med Chem., 2015, 58, 8895); Amine-17, (3S,4R)-1-(4-aminopyrimidin-2-yl)-3-fluoro-4-methylpiperidin-4-ol (Example A64 in WO2014/081718); Amine-18, (R)-2-(5,5-difluoro-1-oxa-7-azaspiro[3.5]nonan-7-yl)pyrimidin-4-amine or (S)-2-(5,5-difluoro-1-oxa-7-azaspiro[3.5]nonan-7-yl)pyrimidin-4-amine (Peak 2 from Preparation 66 and 67); Amine-19, 2-((3S,4R)-3-fluoro-4-methoxypiperidin-1-yl)pyrimidin-4-amine (Preparation 33); Amine-20, (3S,4R)-1-(4-aminopyrimidin-2-yl)-3-fluoropiperidin-4-ol (Example A66 in WO2014/081718).

| Ex no | Name/Structure/RCl/Amine//Purification/Data |
|---|---|
| 45 | (3S,4R)-3-fluoro-1-(4-((5-isopropyl-8-(pyrrolidin-1-yl)-2,7-naphthyridin-3- |
| | yl)amino)pyrimidin-2-yl)-3-methylpiperidin-4-ol |
| | 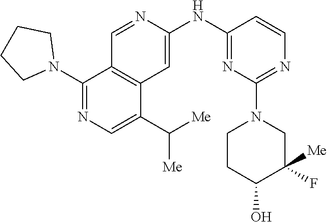 [SMILES: CC(C)c1cnc(N2CCCC2)c2cnc(Nc3ccnc(N4CC[C@@H](O)[C@@](C)(F)C4)n3)cc12] |
| | RCl: 6-chloro-4-isopropyl-1-(pyrrolidin-1-yl)-2,7-naphthyridine (Preparation 157); |
| | Amine-1; BrettPhos Pd G3, 130° C., microwave |
| | Prep-TLC (10:1 DCM/MeOH); HPLC-18, Gradient (% organic): 40-50% |
| | White solid (9.9 mg, 8%); LCMS m/z = 466 [M + H]⁺ ¹HNMR (400 MHz, DMSO- |
| | d₆) δ: 10.01 (s, 1H), 9.31 (s, 1H), 8.43 (s, 1H), 8.03 (d, 1H), 7.96 (s, 1H), 6.52 |
| | (d, 1H), 5.03 (d, 1H), 4.80-4.49 (m, 2H), 3.87-3.67 (m, 4H), 3.64-3.49 (m, |
| | 1H), 3.26-3.02 (m, 2H), 2.03-1.86 (m, 4H), 1.79-1.70 (m, 2H), 1.45-1.21 (m, |
| | 9H). |
| 46 | (3S,4R)-1-(4-((8-(azetidin-1-yl)-5-isopropyl-2,7-naphthyridin-3- |
| | yl)amino)pyrimidin-2-yl)-3-fluoro-3-methylpiperidin-4-ol |
| | 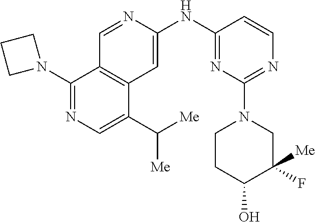 [SMILES: CC(C)c1cnc(N2CCC2)c2cnc(Nc3ccnc(N4CC[C@@H](O)[C@@](C)(F)C4)n3)cc12] |
| | RCl: 1-(azetidin-1-yl)-6-chloro-4-isopropyl-2,7-naphthyridine (Preparation 168); |
| | Amine-1; BrettPhos Pd G3, 100° C. |
| | Prep-TLC (10:1 DCM/MeOH); HPLC-13, Gradient (% organic): 35-60%) |
| | Yellow solid (164.4 mg, 8%); LCMS m/z = 452 [M + H]⁺ ¹HNMR (300 MHz, |
| | DMSO-d₆) δ: 10.02 (s, 1H), 9.03 (s, 1H), 8.45 (s, 1H), 8.03 (d, 1H), 7.97 (s, |
| | 1H), 6.55-6.45 (m, 1H), 5.02 (d, 1H), 4.79-4.61 (m, 2H), 4.38 (t, 4H), 3.68- |
| | 3.49 (m, 1H), 3.28 (s, 1H), 3.15 (q, 2H), 2.40 (p, 2H), 1.73 (s, 2H), 1.46-1.22 |
| | (m, 9H). |
| 47 | (3S,4R)-3-fluoro-1-(4-((8-(3-fluoroazetidin-1-yl)-5-isopropyl-2,7-naphthyridin-3- |
| | yl)amino)pyrimidin-2-yl)-3-methylpiperidin-4-ol |
| | 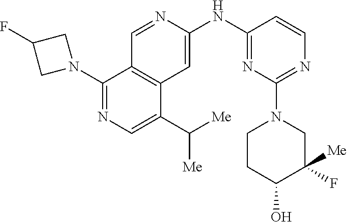 [SMILES: CC(C)c1cnc(N2CC(F)C2)c2cnc(Nc3ccnc(N4CC[C@@H](O)[C@@](C)(F)C4)n3)cc12] |
| | RCl: 6-chloro-1-(3-fluoroazetidin-1-yl)-4-isopropyl-2,7-naphthyridine (Preparation |
| | 278); Amine-1; BrettPhos Pd G3, 100° C. |
| | Prep-TLC (1:2 DCM/MeOH); HPLC-22, Gradient (% organic): 11-14% |
| | White solid (41.4 mg, 35%). LCMS m/z = 470 [M + H]⁺ ¹HNMR (300 MHz, |
| | DMSO-d₆) δ: 10.08 (s, 1H), 9.05 (s, 1H), 8.49 (s, 1H), 8.15-7.88 (m, 2H), |
| | 6.52 (d, 1H), 5.54 (dtt, 1H), 5.03 (d, 1H), 4.81-4.60 (m, 4H), 4.53-4.30 (m, |
| | 2H), 3.64-3.47 (m, 1H), 3.27-2.97 (m, 2H), 1.85-1.68 (m, 2H), 1.46-1.21 (m, |
| | 9H). |
| 48 | (3S,4R)-1-(4-((8-((S)-2-((dimethylamino)methyl)azetidin-1-yl)-5-isopropyl-2,7- |
| | naphthyridin-3-yl)amino)pyrimidin-2-yl)-3-fluoro-3-methylpiperidin-4-ol |
| | trifluoroacetate |
| | 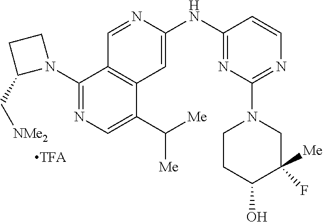 [SMILES: CC(C)c1cnc(N2CC[C@H]2CN(C)C)c2cnc(Nc3ccnc(N4CC[C@@H](O)[C@@](C)(F)C4)n3)cc12] |
| | RCl: (S)-1-(1-(6-chloro-4-isopropyl-2,7-naphthyridin-1-yl)azetidin-2-yl)-N,N- |
| | dimethylmethanamine (Preparation 179); Amine-1; Brettphos(Allyl)Pd OTf, |
| | 100° C. |
| | HPLC-18, Gradient (% organic): 40-50% |
| | Yellow solid (30 mg, 19%). LCMS m/z = 509 [M + H]⁺ ¹HNMR (300 MHz, DMSO- |
| | d₆) δ: 10.04 (s, 1H), 9.02 (s, 1H), 8.46 (s, 1H), 8.14-7.84 (m, 2H), 6.48 (d, |
| | 1H), 5.02 (d, 1H), 4.92-4.54 (m, 4H), 4.09 (q, 1H), 3.54 (dd, 1H), 3.27-2.99 |
| | (m, 3H), 2.80 (dd,1H), 2.60-2.51 (m, 1H), 2.19 (s, 7H), 1.72 (s, 2H), 1.45- |
| | 1.16 (m, 9H). |
| 49 | (3S,4R)-3-fluoro-1-(4-((8-(3-fluoroazetidin-1-yl)-5-isopropyl-2,6-naphthyridin-3- |
| | yl)amino)pyrimidin-2-yl)-3-methylpiperidin-4-ol |
| | 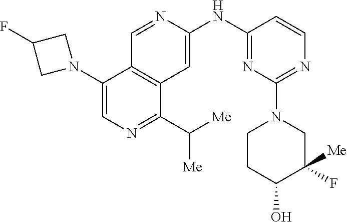 [SMILES: CC(C)c1ncc(N2CC(F)C2)c2cnc(Nc3ccnc(N4CC[C@@H](O)[C@@](C)(F)C4)n3)cc12] |
| | RCl: 7-chloro-4-(3-fluoroazetidin-1-yl)-1-isopropyl-2,6-naphthyridine (Preparation |
| | 344); Amine-1; Brettphos Pd G3, 100° C., |
| | Column Chromatography: (10:1 DCM/MeOH); HPLC-7: Gradient (% organic): 30- |
| | 49%) |
| | Yellow solid. LCMS m/z = 470 [M + H]⁺ ¹HNMR (300 MHz, DMSO-d₆) δ: 10.08 (s, |
| | 1H), 9.13 (s, 1H), 8.72 (s, 1H), 8.03 (d, 1H), 7.65 (s, 1H), 6.47 (d, 1H), 5.55 |
| | (d, 1H), 5.02 (d, 1H), 4.87-4.45 (m, 4H), 4.46-4.24 (m, 2H), 3.78-3.49 (m, |
| | 2H), 3.21-3.02 (m, 2H), 1.82-1.73 (m, 2H), 1.49-1.29 (m, 9H). |
| 50 | (3S,4R)-3-fluoro-1-(4-((8-(3-hydroxy-3-methylazetidin-1-yl)-5-isopropyl-2,7- |
| | naphthyridin-3-yl)amino)pyrimidin-2-yl)-3-methylpiperidin-4-ol |
| | 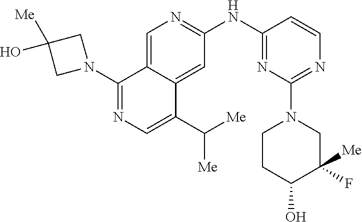 [SMILES: CC(C)c1cnc(N2CC(C)(O)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-3-methylazetidin-3-ol |
| | (Preparation 183); Amine-1; Brettphos Pd G3, 100° C. |
| | Column Chromatography: (10:1 DCM/MeOH); HPLC-18, Gradient (% organic): |
| | 25-40% |
| | White solid (108 mg, 55%). LCMS m/z = 482 [M + H]⁺ ¹HNMR (300 MHz, DMSO- |
| | d₆) δ: 10.04 (s, 1H), 9.02 (s, 1H), 8.46 (s, 1H), 8.01 (d, 1H,), 7.96 (s, 1H), 6.49 |
| | (d, 1H), 5.58 (s, 1H), 5.01 (d, 1H), 4.81-4.56 (m, 2H), 4.36-4.03 (m, 4H), |
| | 3.74-3.37 (m, 2H), 3.24-2.97 (m, 2H), 1.78-1.64 (m, 2H), 1.55-1.15 (m, 12H). |
| 51 | (3S,4R)-3-fluoro-1-(4-((5-isopropyl-8-(3-methoxyazetidin-1-yl)-2,7-naphthyridin-3- |
| | yl)amino)pyrimidin-2-yl)-3-methylpiperidin-4-ol |
| | 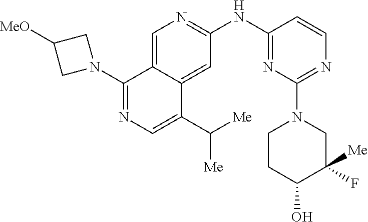 [SMILES: COC1CN(c2ncc(C(C)C)c3cc(Nc4ccnc(N5CC[C@@H](O)[C@@](C)(F)C5)n4)ncc23)C1] |
| | RCl: 6-chloro-4-isopropyl-1-(3-methoxyazetidin-1-yl)-2,7-naphthyridine |
| | (Preparation 181); Amine-1; Brettphos Pd G3, 100° C., |
| | Column Chromatography: (10:1 DCM/MeOH); HPLC-18, Gradient (% organic): |
| | 29-44% |
| | White solid (75 mg, 38%). LCMS m/z = 482 [M + H]⁺ ¹HNMR (300 MHz, DMSO- |
| | d₆) δ: 10.03 (s, 1H), 9.03 (s, 1H), 8.45 (s, 1H), 8.02 (d, 1H), 7.96 (s, 1H), 6.51 |
| | (d, 1H), 5.01 (d, 1H), 4.86-4.42 (m, 4H), 4.40-4.27 (m, 1H), 4.18 (dd, 2H), |
| | 3.65-3.46 (m, 1H), 3.37-3.29 (m, 4H), 3.20-2.91 (m, 2H), 1.84-1.62 (m, 2H), |
| | 1.45-1.26 (m, 9H). |
| 52 | (3S,4R)-3-fluoro-1-(4-((8-((S)-2-(hydroxymethyl)azetidin-1-yl)-5-isopropyl-2,7- |
| | naphthyridin-3-yl)amino)pyrimidin-2-yl)-3-methylpiperidin-4-ol |
| | 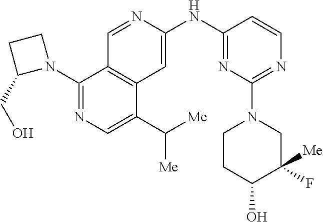 [SMILES: CC(C)c1cnc(N2CC[C@H]2CO)c2cnc(Nc3ccnc(N4CC[C@@H](O)[C@@](C)(F)C4)n3)cc12] |
| | RCl: (S)-(1-(6-chloro-4-isopropyl-2,7-naphthyridin-1-yl)azetidin-2-yl)methanol |
| | (Preparation 195); Amine-1; Brettphos Pd G3, 100° C., |
| | Column Chromatography: (10:1 DCM/MeOH) |
| | HPLC-21, Gradient (% organic): 28-42% |
| | Yellow solid (22.3 mg, 27%). LCMS m/z = 482 [M + H]⁺ ¹HNMR (400 MHz, |
| | DMSO-d₆) δ: 10.06 (s, 1H), 9.05 (s, 1H), 8.46 (s, 1H), 8.03 (d, 1H), 7.93 (s, |
| | 1H), 6.51 (d, 1H), 5.23 (t, 1H), 5.04 (d, 1H), 4.82-4.54 (m, 4H), 4.23 (q, 1H), |
| | 3.71 (t, 2H), 3.63-3.43 (m, 1H), 3.14 (q, 2H), 2.40-2.19 (m, 2H), 1.84-1.61 |
| | (m, 2H), 1.50-1.22 (m, 9H). |
| 53 | (3S,4R)-3-fluoro-1-(4-((5-isopropyl-8-(6-oxa-1-azaspiro[3.3]heptan-1-yl)-2,7- |
| | naphthyridin-3-yl)amino)pyrimidin-2-yl)-3-methylpiperidin-4-ol |
| | 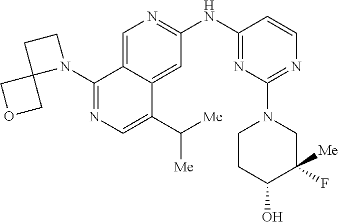 [SMILES: CC(C)c1cnc(N2CCC23COC3)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-6-oxa-1-azaspiro[3.3]heptane |
| | (Preparation 173); Amine-1; Brettphos Pd G3, 100° C. |
| | HPLC-17, Gradient (% organic): 25-50% |
| | Yellow solid (10 mg, 8%). LCMS m/z = 494 [M + H]⁺ ¹HNMR (300 MHz, DMSO- |
| | d₆) δ: 10.02 (s, 1H), 9.12 (s, 1H), 8.44 (s, 1H), 8.03 (d, 2H), 6.51(d, 1H), 5.66 |
| | (d, 2H), 5.05 (d, 1H), 4.83-4.57 (m, 2H), 4.54 (dd, 4H), 3.56 (dq, 2H), 3.23- |
| | 2.95 (m, 2H), 2.71 (t, 2H), 1.82-1.63 (m, 2H), 1.45-1.25 (m, 9H). |
| 54 | (3S,4R)-3-fluoro-1-(4-((5-isopropyl-8-(1-oxa-6-azaspiro[3.3]heptan-6-yl)-2,7- |
| | naphthyridin-3-yl)amino)pyrimidin-2-yl)-3-methylpiperidin-4-ol |
| | 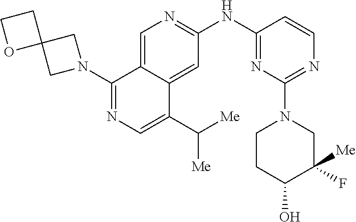 [SMILES: CC(C)c1cnc(N2CC3(CCO3)C2)c2cnc(Nc3ccnc(N4CC[C@@H](O)[C@@](C)(F)C4)n3)cc12] |
| | RCl: 6-(6-chloro-4-isopropyl-2,7-naphthyridin-1-yl)-1-oxa-6-azaspiro[3.3]heptane |
| | (Preparation 174); Amine-1; Brettphos Pd G3, 100° C. |
| | HPLC-18, Gradient (% organic): 30-41% |
| | Yield: 31.5 mg, 35%. LCMS m/z = 494 [M + H]⁺ ¹HNMR (300 MHz, DMSO-d₆) δ: |
| | 10.10 (s, 1H), 9.05 (s, 1H), 8.49 (s, 1H), 8.21-7.79 (m, 2H), 6.50 (d, 1H), 5.05 |
| | (d, 1H), 4.90-4.11 (m, 7H), 3.78-3.42 (m, 2H), 3.20-2.99 (m, 2H), 2.96-2.71 |
| | (m, 2H), 1.74 (s, 2H), 1.51-1.03 (m, 9H). |
| 55 | (3S,4R)-1-(4-((8-(3-(difluoromethyl)azetidin-1-yl)-5-isopropyl-2,7-naphthyridin-3- |
| | yl)amino)pyrimidin-2-yl)-3-fluoro-3-methylpiperidin-4-ol |
| | 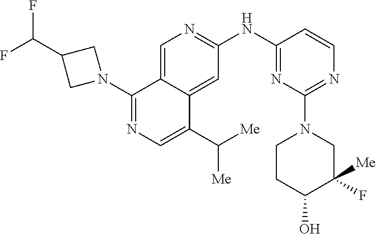 [SMILES: CC(C)c1cnc(N2CC(C(F)F)C2)c2cnc(Nc3ccnc(N4CC[C@@H](O)[C@@](C)(F)C4)n3)cc12] |
| | RCl: 6-chloro-1-(3-(difluoromethyl)azetidin-1-yl)-4-isopropyl-2,7-naphthyridine |
| | (Preparation 185); Amine-1; Brettphos Pd G3, 100° C., |
| | HPLC-10: Gradient (% organic): 40-65% |
| | Yield: 30 mg, 19%. LCMS m/z = 502 [M + H]⁺ ¹HNMR (300 MHz, DMSO-d₆) δ: |
| | 10.09 (s, 1H), 9.04 (s, 1H), 8.48 (s, 1H), 8.12-7.96 (m, 2H), 6.65-6.22 (m, |
| | 2H), 5.03 (d, 1H), 4.82-4.58 (m, 2H), 4.47 (t, 2H), 4.38-4.19 (m, 2H), 3.53 |
| | (dt, 1H), 3.29 (s, 1H), 3.19-3.01 (m, 3H), 1.72 (s, 2H), 1.47-1.15 (m, 9H). |
| 56 | (3S,4R)-1-(4-((8-(cis-3,4-difluoropyrrolidin-1-yl)-5-isopropyl-2,7-naphthyridin-3- |
| | yl)amino)pyrimidin-2-yl)-3-fluoro-3-methylpiperidin-4-ol |
| | 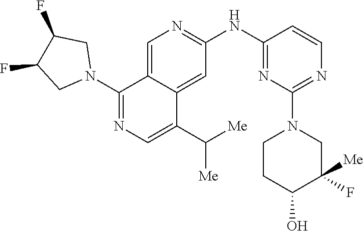 [SMILES: CC(C)c1cnc(N2C[C@@H](F)[C@@H](F)C2)c2cnc(Nc3ccnc(N4CC[C@@H](O)[C@@](C)(F)C4)n3)cc12] |
| | RCl: 6-chloro-1-(cis-3,4-difluoropyrrolidin-1-yl)-4-isopropyl-2,7-naphthyridine |
| | (Preparation 219); Amine-1; Brettphos Pd G3, 100° C. |
| | HPLC-18, Gradient (% organic): 40-48% |
| | Yellow solid (57 mg, 35%). LCMS m/z = 502 [M + H]⁺ ¹HNMR (300 MHz, DMSO- |
| | d₆) δ: 9.95 10.12 (s, 1H), 9.26 (s, 1H), 8.47 (s, 1H), 8.02 (d, 1H), 7.97 (s, 1H), |
| | 6.50 (d, 1H), 5.57-5.19 (m, 2H), 5.03 (d, 1H), 4.79-4.52 (m, 2H), 4.29-3.89 |
| | (m, 4H), 3.64-3.39 (m, 1H), 3.39-3.25 (m, 2H), 3.13 (q, 2H), 1.84-1.63 (m, |
| | 2H), 1.44-1.25 (m, 9H). |
| 57 | (3S,4R)-1-(4-((8-(3,3-difluoropyrrolidin-1-yl)-5-isopropyl-2,7-naphthyridin-3- |
| | yl)amino)pyrimidin-2-yl)-3-fluoro-3-methylpiperidin-4-ol |
| | 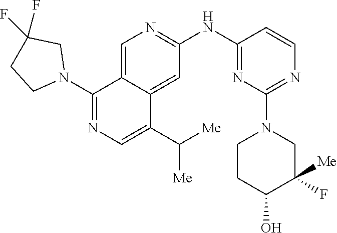 [SMILES: CC(C)c1cnc(N2CCC(F)(F)C2)c2cnc(Nc3ccnc(N4CC[C@@H](O)[C@@](C)(F)C4)n3)cc12] |
| | RCl: 6-chloro-1-(3,3-difluoropyrrolidin-1-yl)-4-isopropyl-2,7-naphthyridine |
| | (Preparation 177); Amine-1; Brettphos Pd G3, 100° C., |
| | HPLC-16, Gradient (% organic): 25-50% |
| | Yellow solid (57 mg, 35%). LCMS m/z = 502 [M + H]⁺; ¹HNMR (400 MHz, |
| | DMSO-d₆) δ: 10.15 (s, 1H), 9.29 (s, 1H), 8.49 (s, 1H), 8.10-7.91 (m, 2H), |
| | 6.52 (d, 1H), 5.05 (d, 1H), 4.84-4.51 (m, 2H), 4.18 (t, 2H), 4.02 (t, 2H), 3.70- |
| | 3.44 (m, 2H), 3.15 (d, 2H), 2.63-2.49 (m, 2H), 1.84-1.58 (m, 2H), 1.44-1.28 |
| | (m, 9H). |
| 58 | 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidine-3-carbonitrile |
| | 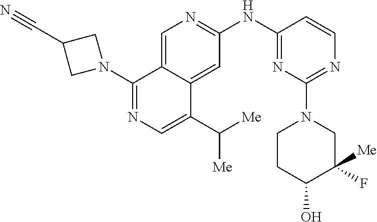 [SMILES: CC(C)c1cnc(N2CC(C#N)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)azetidine-3-carbonitrile |
| | (Preparation 159); Amine-1; Brettphos Pd G3, 100 C, |
| | Column Chromatography (10:1 DCM/MeOH); HPLC-12: Gradient (% organic): 30- |
| | 57%). |
| | Yellow solid (4 mg, 2.4%). LCMS m/z = 477 [M + H]⁺; ¹HNMR (300 MHz, DMSO- |
| | d₆) δ: 10.11 (s, 1H), 9.02 (s, 1H), 8.50 (s, 1H), 8.06-7.97 (m, 2H), 6.55-6.45 |
| | (m, 1H), 5.03 (d, 1H), 4.71 (s, 1H), 4.62 (t, 3H), 4.52 (t, 2H), 3.92 (ddd, 1H), |
| | 3.6(s,2H),3.2(s,2H), 1.72 (s, 2H), 1.41-1.26 (m, 9H). |
| 59 | (3S,4R)-1-(4-((8-(1,1-difluoro-5-azaspiro[2.3]hexan-5-yl)-5-isopropyl-2,7- |
| | naphthyridin-3-yl)amino)pyrimidin-2-yl)-3-fluoro-3-methylpiperidin-4-ol |
| | 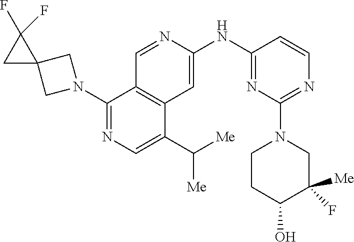 [SMILES: CC(C)c1cnc(N2CC3(C2)CC3(F)F)c2cnc(Nc3ccnc(N4CC[C@@H](O)[C@@](C)(F)C4)n3)cc12] |
| | RCl: 6-chloro-1-(1,1-difluoro-5-azaspiro[2.3]hexan-5-yl)-4-isopropyl-2,7- |
| | naphthyridine (Preparation 186); Amine-1; RuPhos Pd G3, 100° C., |
| | HPLC-18, Gradient (% organic): 73-75% |
| | White solid (3.1 mg, 1%). LCMS m/z = 514 [M + H]⁺; ¹HNMR (400 MHz, DMSO- |
| | d₆) δ: 10.15 (s, 1H), 9.07 (s, 1H), 8.52 (s, 1H), 8.18-7.87 (m, 2H), 6.50 (d, |
| | 1H), 5.05 (d, 1H), 4.81-4.57 (m, 2H), 4.64-4.42 (m, 4H), 3.73-3.49 (m, 2H), |
| | 3.15 (d, 2H), 1.77 (dd, 4H), 1.51-1.24 (m, 9H) |
| 60 | (3S,4R)-1-(4-((8-((3R,4R)-3,4-difluoropyrrolidin-1-yl)-5-isopropyl-2,7- |
| | naphthyridin-3-yl)amino)pyrimidin-2-yl)-3-fluoro-3-methylpiperidin-4-ol or |
| | (3S,4R)-1-(4-((8-((3S,4S)-3,4-difluoropyrrolidin-1-yl)-5-isopropyl-2,7- |
| | naphthyridin-3-yl)amino)pyrimidin-2-yl)-3-fluoro-3-methylpiperidin-4-ol |
| | 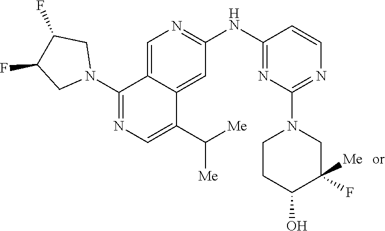 [SMILES: CC(C)c1cnc(N2C[C@@H](F)[C@H](F)C2)c2cnc(Nc3ccnc(N4CC[C@@H](O)[C@@](C)(F)C4)n3)cc12] |
| | 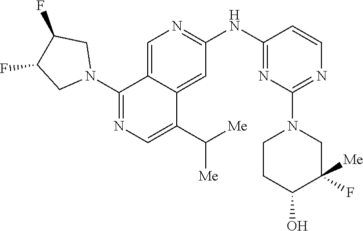 [SMILES: CC(C)c1cnc(N2C[C@H](F)[C@@H](F)C2)c2cnc(Nc3ccnc(N4CC[C@@H](O)[C@@](C)(F)C4)n3)cc12] |
| | RCl: 6-chloro-1-((3R,4R)-3,4-difluoropyrrolidin-1-yl)-4-isopropyl-2,7- |
| | naphthyridine or 6-chloro-1-((3S,4S)-3,4-difluoropyrrolidin-1-yl)-4- |
| | isopropyl-2,7-naphthyridine (Peak 1 from Preparation 221 and 222); Amine- |
| | 1; RuPhos Pd G3, 100° C. |
| | Column Chromatography (10:1 DCM/MeOH); HPLC-27, Gradient (% organic): 10- |
| | 20% |
| | Yellow solid (19 mg, 23%). LCMS m/z = 502 [M + H]⁺; ¹HNMR (300 MHz, |
| | DMSO-d₆) δ: 10.11 (s, 1H), 9.30 (s, 1H), 8.48 (s, 1H), 8.02 (d, 1H), 7.98 (s, |
| | 1H), 6.50 (d, 1H), 5.55 (d, 1H), 5.36 (d, 1H), 5.01 (s, 1H), 4.82-4.60 (m, 2H), |
| | 4.44 (dd, 1H), 4.29 (d, 1H), 3.91 (dd, 2H), 3.54 (dt, 1H), 3.33 (p, 1H), 3.23- |
| | 3.02 (m, 2H), 1.72 (d, 2H), 1.44-1.25 (m, 9H). |
| 61 | (3S,4R)-3-fluoro-1-(4-((8-(3-(hydroxymethyl)azetidin-1-yl)-5-isopropyl-2,7- |
| | naphthyridin-3-yl)amino)pyrimidin-2-yl)-3-methylpiperidin-4-ol |
| | 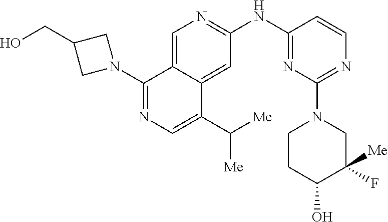 [SMILES: CC(C)c1cnc(N2CC(CO)C2)c2cnc(Nc3ccnc(N4CC[C@@H](O)[C@@](C)(F)C4)n3)cc12] |
| | RCl: (1-(6-chloro-4-isopropyl-2,7-naphthyridin-1-yl)azetidin-3-yl)methanol |
| | (Preparation 162); Amine-1; BrettPhos Pd G3, 100 C |
| | Column Chromatography (10:1 DCM/MeOH); HPLC-12, Gradient (% organic): 30- |
| | 57%) |
| | White solid (36 mg, 22%). LCMS m/z = 482 [M + H]⁺; ¹HNMR (300 MHz, DMSO- |
| | d₆) δ: 10.07 (s, 1H), 9.05 (s, 1H), 8.47 (s, 1H), 8.03 (d, 1H), 7.97 (s, 1H), 6.50 |
| | (d, 1H), 5.05 (d, 1H), 4.81 (t, 1H), 4.69 (t, 2H), 4.40 (t, 2H), 4.17-4.01 (m, |
| | 2H), 3.75-3.44 (m, 3H), 3.23-2.93 (m, 3H), 2.94-2.79 (m, 1H), 1.82-1.67 (m, |
| | 2H), 1.46-1.26 (m, 9H) |
| 62 | (3S,4R)-3-fluoro-1-(4-((8-(3-hydroxyazetidin-1-yl)-5-isopropyl-2,7-naphthyridin-3- |
| | yl)amino)pyrimidin-2-yl)-3-methylpiperidin-4-ol |
| | 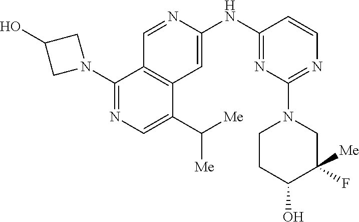 [SMILES: CC(C)c1cnc(N2CC(O)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)azetidin-3-ol (Preparation 161); |
| | Amine-1; BrettPhos Pd G3, 100° C. |
| | HPLC-18, Gradient (% organic): 23-35% |
| | White solid (35 mg, 9%). LCMS m/z = 468 [M + H]⁺; ¹HNMR (400 MHz, DMSO- |
| | d₆) δ: 10.05 (s, 1H), 9.03 (d, 1H), 8.46 (s, 1H), 8.03 (d, 1H), 7.97 (s, 1H), 6.51 |
| | (d, 1H), 5.69 (d, 1H), 5.04 (d, 1H), 4.76-4.44 (m, 5H), 4.17-4.04 (m, 2H), |
| | 3.66-3.43 (m, 1H), 3.13 (t, 2H), 1.81-1.61 (m, 2H), 1.42-1.28 (m, 9H) |
| 63 | (3S,4R)-1-(4-((8-(1,1-difluoro-5-azaspiro[2.4]heptan-5-yl)-5-isopropyl-2,7- |
| | naphthyridin-3-yl)amino)pyrimidin-2-yl)-3-fluoro-3-methylpiperidin-4-ol |
| | 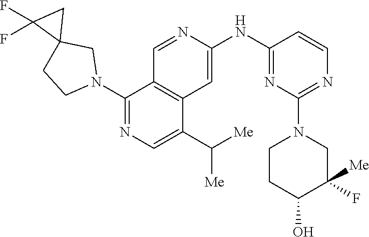 [SMILES: CC(C)c1cnc(N2CCC3(C2)CC3(F)F)c2cnc(Nc3ccnc(N4CC[C@@H](O)[C@@](C)(F)C4)n3)cc12] |
| | RCl: 6-chloro-1-(1,1-difluoro-5-azaspiro[2.4]heptan-5-yl)-4-isopropyl-2,7- |
| | naphthyridine (Preparation 212); Amine-1; XPhos Pd G4, 100° C. |
| | LCMS m/z = 528 [M + H]⁺ |
| 64 | (3S,4R)-3-fluoro-1-(4-((5-isopropyl-8-(6-(2-methoxyethyl)-1,6- |
| | diazaspiro[3.3]heptan-1-yl)-2,7-naphthyridin-3-yl)amino)pyrimidin-2-yl)-3- |
| | methylpiperidin-4-ol |
| | 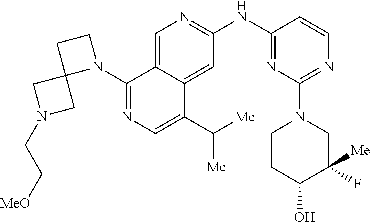 [SMILES: COCCN1CC2(CCN2c2ncc(C(C)C)c3cc(Nc4ccnc(N5CC[C@@H](O)[C@@](C)(F)C5)n4)ncc23)C1] |
| | RCl: 6-chloro-4-isopropyl-1-(6-(2-methoxyethyl)-1,6-diazaspiro[3.3]heptan-1-yl)- |
| | 2,7-naphthyridine (Preparation 238); Amine-1; BrettPhos Pd G3, 100 C |
| | HPLC-2 Gradient (% organic): 31-55%) |
| | Yellow solid (20 mg, 26%). LCMS m/z = 551 [M + H]⁺; ¹HNMR (300 MHz, |
| | DMSO-d₆) δ: 10.02 (s, 1H), 9.14 (s, 1H), 8.43 (s, 1H), 8.03 (d, 1H), 7.97 (s, |
| | 1H), 6.51 (d, 1H), 5.05 (d, 1H), 4.83-4.46 (m, 4H), 4.35 (d, 2H), 3.67-3.45 |
| | (m, 4H), 3.23 (s, 3H), 3.16-3.02 (m, 2H), 2.80 (t, 2H), 2.64 (t, 2H), 1.84-1.61 |
| | (m, 3H), 1.42-1.25 (m, 9H) |
| 65 | (3S,4R)-3-fluoro-1-(4-((8-((S)-2-((R)-1-hydroxyethyl)azetidin-1-yl)-5-isopropyl- |
| | 2,7-naphthyridin-3-yl)amino)pyrimidin-2-yl)-3-methylpiperidin-4-ol or |
| | (3S,4R)-3-fluoro-1-(4-((8-((S)-2-((S)-1-hydroxyethyl)azetidin-1-yl)-5- |
| | isopropyl-2,7-naphthyridin-3-yl)amino)pyrimidin-2-yl)-3-methylpiperidin-4- |
| | ol |
| | 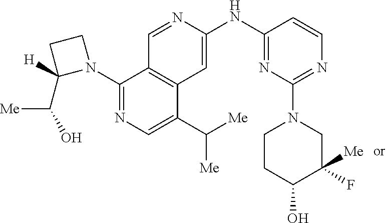 [SMILES: CC(C)c1cnc(N2CC[C@H]2[C@@H](C)O)c2cnc(Nc3ccnc(N4CC[C@@H](O)[C@@](C)(F)C4)n3)cc12] |
| | 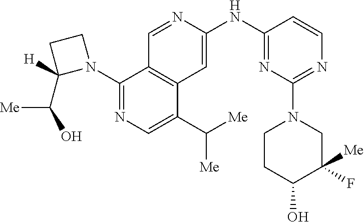 [SMILES: CC(C)c1cnc(N2CC[C@H]2[C@H](C)O)c2cnc(Nc3ccnc(N4CC[C@@H](O)[C@@](C)(F)C4)n3)cc12] |
| | RCl: 1-((S)-1-(6-chloro-4-isopropyl-2,7-naphthyridin-1-yl)azetidin-2-yl)ethan-1-ol |
| | (Preparation 242); Amine-1; BrettPhos Pd G3, 100° C. |
| | HPLC-21, Gradient (% organic): 34-40% |
| | Peak 2; White solid (5 mg, 12%). LCMS m/z = 496 [M + H]⁺; ¹HNMR (300 MHz, |
| | DMSO-d₆) δ: 10.10 (s, 1H), 9.09 (s, 1H), 8.47 (s, 1H), 8.04 (d, 1H), 7.88 (s, |
| | 1H), 6.52 (d, 1H), 6.24 (s, 1H), 5.04 (d, 1H), 4.80-4.57 (m, 3H), 4.52 (q, 1H), |
| | 4.28 (d, 1H), 4.02-3.89 (m, 1H), 3.56 (dd, 1H), 3.30 (m, 1H), 3.13 (t, 2H), |
| | 2.40-2.12 (m, 2H), 1.74 (d, 2H), 1.43-1.24 (m, 9H), 1.07 (d, 3H) |
| 66 | (3S,4R)-3-fluoro-1-(4-((5-isopropyl-8-(3-(methoxymethyl)azetidin-1-yl)-2,7- |
| | naphthyridin-3-yl)amino)pyrimidin-2-yl)-3-methylpiperidin-4-ol |
| | 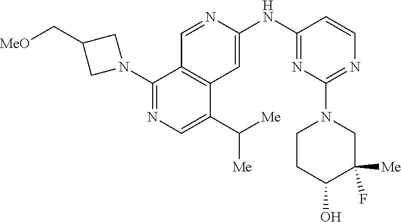 [SMILES: COCC1CN(c2ncc(C(C)C)c3cc(Nc4ccnc(N5CC[C@@H](O)[C@@](C)(F)C5)n4)ncc23)C1] |
| | RCl: 6-chloro-4-isopropyl-1-(3-(methoxymethyl)azetidin-1-yl)-2,7-naphthyridine |
| | (Preparation 196); Amine-1; BrettPhos Pd G3, 100° C. |
| | HPLC-21, Gradient (% organic): 31-51% |
| | White solid (20 mg, 11%). LCMS m/z = 496 [M + H]⁺; ¹HNMR (300 MHz, DMSO- |
| | d₆) δ: 10.06 (s, 1H), 9.02 (s, 1H), 8.45 (s, 1H), 8.01 (d, 1H), 7.95 (s, 1H), 6.48 |
| | (d, 1H), 5.03 (d, 1H), 4.80-4.54 (m, 2H), 4.41 (t, 2H), 4.08 (dd, 2H), 3.64- |
| | 3.42 (m, 3H), 3.31 (s, 3H), 3.18-2.92 (m, 3H), 1.88-1.56 (m, 2H), 1.42-1.21 |
| | (m, 9H) |
| 67 | (3S,4R)-3-fluoro-1-(4-((8-(3-fluoro-3-(hydroxymethyl)azetidin-1-yl)-5-isopropyl- |
| | 2,7-naphthyridin-3-yl)amino)pyrimidin-2-yl)-3-methylpiperidin-4-ol |
| | 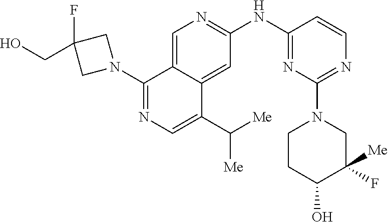 [SMILES: CC(C)c1cnc(N2CC(F)(CO)C2)c2cnc(Nc3ccnc(N4CC[C@@H](O)[C@@](C)(F)C4)n3)cc12] |
| | RCl: (1-(6-chloro-4-isopropyl-2,7-naphthyridin-1-yl)-3-fluoroazetidin-3- |
| | yl)methanol (Preparation 192); Amine-1; BrettPhos Pd G3, 100° C. |
| | HPLC-12, Gradient (% organic): 30-57% |
| | White solid (20 mg, 12%). LCMS m/z = 500 [M + H]⁺; ¹HNMR (300 MHz, DMSO- |
| | d₆) δ: 10.11 (s, 1H), 9.04 (s, 1H), 8.49 (s, 1H), 8.02 (d, 1H), 7.99 (s, 1H), 6.48 |
| | (d, 1H), 5.31 (t, 1H), 5.03 (d, 1H), 4.70 (dd, 2H), 4.55-4.27 (m, 4H), 3.74 (dd, |
| | 2H), 3.53 (d, 1H), 3.21-3.02 (m, 2H), 1.78-1.66 (m, 2H), 1.39-1.26 (m, 9H) |
| 68 | (3S,4R)-3-fluoro-1-(4-((8-(3-fluoro-3-(methoxymethyl)azetidin-1-yl)-5-isopropyl- |
| | 2,7-naphthyridin-3-yl)amino)pyrimidin-2-yl)-3-methylpiperidin-4-ol |
| | 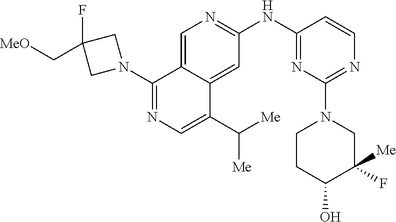 [SMILES: COCC1(F)CN(c2ncc(C(C)C)c3cc(Nc4ccnc(N5CC[C@@H](O)[C@@](C)(F)C5)n4)ncc23)C1] |
| | RCl: 6-chloro-1-(3-fluoro-3-(methoxymethyl)azetidin-1-yl)-4-isopropyl-2,7- |
| | naphthyridine (Preparation 193); Amine-1; BrettPhos Pd G3, 100° C.; |
| | HPLC-12, Gradient (% organic): 30-57% |
| | White solid (20 mg, 13%). LCMS m/z = 514 [M + H]⁺; ¹HNMR (300 MHz, DMSO- |
| | d₆) δ: 10.11 (s, 1H), 9.04 (s, 1H), 8.50 (s, 1H), 8.08-7.96 (m, 2H), 6.48 (d, |
| | 1H), 5.03 (d, 1H), 4.69 (dd, 2H), 4.47 (dd, 4H), 3.79 (s, 1H), 3.72 (s, 1H), |
| | 3.53 (d, 1H), 3.35 (s, 3H), 3.20-3.03 (m, 2H), 1.79-1.62 (m, 2H), 1.44-1.22 |
| | (m, 9H) |
| 69 | (3S,4R)-3-fluoro-1-(4-((5-isopropyl-8-(3-(methylsulfonyl)azetidin-1-yl)-2,7- |
| | naphthyridin-3-yl)amino)pyrimidin-2-yl)-3-methylpiperidin-4-ol |
| | 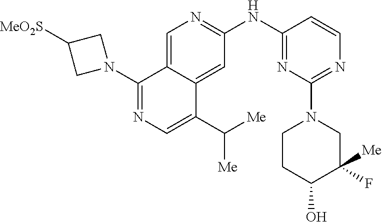 [SMILES: CC(C)c1cnc(N2CC(S(C)(=O)=O)C2)c2cnc(Nc3ccnc(N4CC[C@@H](O)[C@@](C)(F)C4)n3)cc12] |
| | RCl: 6-chloro-4-isopropyl-1-(3-(methylsulfonyl)azetidin-1-yl)-2,7-naphthyridine |
| | (Preparation 207); Amine-1; XPhos Pd G4, 100° C. |
| | White solid (80 mg, 65%). LCMS m/z = 530 [M + H]⁺; ¹HNMR (500 MHz, DMSO- |
| | d₆) δ: 10.18 (s, 1H), 9.12 (s, 1H), 8.57 (s, 1H), 8.19-7.99 (m, 2H), 6.57 (d, |
| | 1H), 5.08 (d, 1H), 4.90-4.60 (m, 5H), 4.54 (dq, 1H), 3.72-3.52 (m, 1H), 3.17 |
| | (s, 5H), 1.80 (q, 2H), 1.49-1.32 (m, 8H). |
| 70 | N-((1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-2-yl)methyl)acetamide |
| | 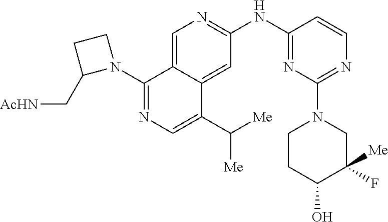 [SMILES: CC(=O)NCC1CCN1c1ncc(C(C)C)c2cc(Nc3ccnc(N4CC[C@@H](O)[C@@](C)(F)C4)n3)ncc12] |
| | RCl: N-((1-(6-chloro-4-isopropyl-2,7-naphthyridin-1-yl)azetidin-2- |
| | yl)methyl)acetamide (Preparation 163); Amine-1; XPhos Pd G4, 100° C. |
| | HPLC-2: Gradient (% organic): 20-40%) |
| | White solid (40 mg, 29%). LCMS m/z = 523 [M + H]⁺; ¹HNMR (400 MHz, DMSO- |
| | d₆) δ: 10.10 (s, 1H), 9.06 (s, 1H), 8.49 (s, 1H), 8.10 (t, 1H), 8.04 (d, 1H), 7.99 |
| | (s, 1H), 6.50 (d, 1H), 5.06 (d, 1H), 4.70 (d, 4H), 4.25-4.07 (m, 1H), 3.63-3.38 |
| | (m, 3H), 3.13 (s, 3H), 2.41-2.15 (m, 2H), 1.79-1.62 (m, 2H), 1.46-1.26 (m, |
| | 9H) |
| 71 | 2-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-7-methyl-5-oxa-2,7- |
| | diazaspiro[3.4]octan-6-one |
| | 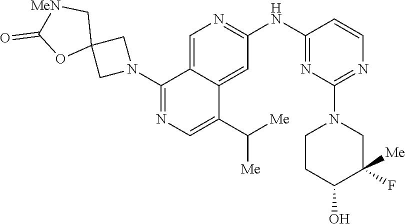 [SMILES: CC(C)c1cnc(N2CC3(CN(C)C(=O)O3)C2)c2cnc(Nc3ccnc(N4CC[C@@H](O)[C@@](C)(F)C4)n3)cc12] |
| | RCl: 2-(6-chloro-4-isopropyl-2,7-naphthyridin-1-yl)-7-methyl-5-oxa-2,7- |
| | diazaspiro[3.4]octan-6-one (Preparation 218); Amine-1; XPhos Pd G4 90° C. |
| | RP-ISCO: 0-40% MeCN/H2O (+0.1% TFA) followed by neuralisation and |
| | extraction. Off-white solid (13 mg, 18%). LCMS m/z = 523 [M + H]⁺; |
| | ¹HNMR (400 MHz, DMSO-d₆) δ: 10.08 (s, 1H), 9.03 (s, 1H), 8.49 (s, 1H), |
| | 8.08-7.93 (m, 2H), 6.48 (d, 1H), 5.00 (d, 1H), 4.75-4.51 (m, 6H), 3.82 (s, 2H), |
| | 3.62-3.46 (m, 1H), 3.19-3.04 (m, 2H), 2.76 (s, 3H), 1.72 (br.s, 2H), 1.49-1.16 |
| | (m,11H). |
| 72 | 2-(1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-3-hydroxyazetidin-3- |
| | yl)acetonitrile |
| | 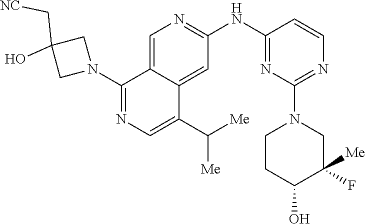 [SMILES: CC(C)c1cnc(N2CC(O)(CC#N)C2)c2cnc(Nc3ccnc(N4CC[C@@H](O)[C@@](C)(F)C4)n3)cc12] |
| | RCl: 2-(1-(6-chloro-4-isopropyl-2,7-naphthyridin-1-yl)-3-hydroxyazetidin-3- |
| | yl)acetonitrile (Preparation 205); Amine-1; XPhos Pd G4, 100° C. |
| | LCMS m/z = 507 [M + H]⁺; ¹HNMR (500 MHz, DMSO-d₆) δ: 10.09 (s, 1H), 9.04 (s, |
| | 1H), 8.50 (s, 1H), 8.10-7.91 (m, 2H), 6.50 (d, 1H), 6.38 (s, 1H), 5.02 (d, 1H), |
| | 4.81-4.56 (m, 2H), 4.39 (d, 2H), 4.26 (d, 2H), 3.74-3.46 (m, 1H), 3.11 (d, |
| | 4H), 1.80-1.65 (m, 2H), 1.48-1.21 (m, 10H). |
| 73 | 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-3-(hydroxymethyl)azetidine-3- |
| | carbonitrile |
| | 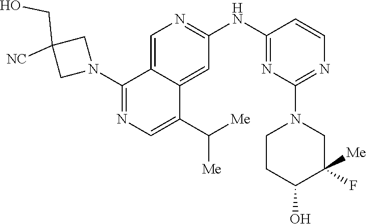 [SMILES: CC(C)c1cnc(N2CC(C#N)(CO)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-3-(hydroxymethyl)azetidine-3- |
| | carbonitrile (Preparation 206); Amine-1; XPhos Pd G4, 100° C. |
| | LCMS m/z = 507 [M + H]⁺ |
| 74 | (3S,4R)-3-fluoro-1-(4-((5-isopropyl-8-((R)-3-(methylsulfonyl)pyrrolidin-1-yl)-2,7- |
| | naphthyridin-3-yl)amino)pyrimidin-2-yl)-3-methylpiperidin-4-ol |
| | 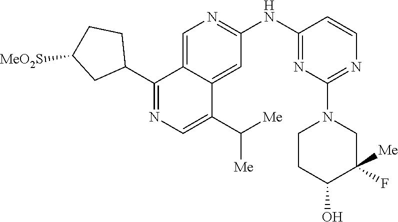 [SMILES: CC(C)c1cnc(C2CC[C@@H](S(C)(=O)=O)C2)c2cnc(Nc3ccnc(N4CC[C@@H](O)[C@@](C)(F)C4)n3)cc12] |
| | RCl: (R)-6-chloro-4-isopropyl-1-(3-(methylsulfonyl)pyrrolidin-1-yl)-2,7- |
| | naphthyridine (Preparation 209); Amine-1; XPhos Pd G4, 100° C. |
| | LCMS m/z = 544 [M + H]⁺; ¹HNMR (500 MHz, DMSO-d₆) δ: 10.07 (s, 1H), 9.30 (s, |
| | 1H), 8.47 (s, 1H), 8.13-7.89 (m, 2H), 6.52 (d, 1H), 5.02 (d, 1H), 4.85-4.51 (m, |
| | 2H), 4.25-4.02 (m, 3H), 4.05-3.75 (m, 2H), 3.71-3.41 (m, 1H), 3.09 (s, 5H), |
| | 2.37 (q, 2H), 1.73 (s, 2H), 1.43-1.25 (m, 9H). |
| 75 | (3S,4R)-3-fluoro-1-(4-((5-isopropyl-8-((S)-3-(methylsulfonyl)pyrrolidin-1-yl)-2,7- |
| | naphthyridin-3-yl)amino)pyrimidin-2-yl)-3-methylpiperidin-4-ol |
| | 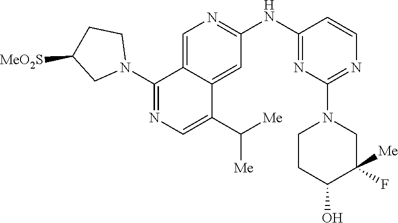 [SMILES: CC(C)c1cnc(N2CC[C@H](S(C)(=O)=O)C2)c2cnc(Nc3ccnc(N4CC[C@@H](O)[C@@](C)(F)C4)n3)cc12] |
| | RCl: (S)-6-chloro-4-isopropyl-1-(3-(methylsulfonyl)pyrrolidin-1-yl)-2,7- |
| | naphthyridine (Preparation 208); Amine-1; XPhos Pd G4, 100° C. |
| | LCMS m/z = 507 [M + H]⁺; ¹HNMR (500 MHz, DMSO-d₆) δ: 10.00 (s, 1H), 9.23 (s, |
| | 1H), 8.40 (s, 1H), 8.04-7.85 (m, 2H), 6.45 (d, 1H), 4.96 (d, 1H), 4.77-4.50 (m, |
| | 2H), 4.12-3.96 (m, 3H), 3.96-3.77 (m, 1H), 3.49 (dq, 1H), 3.02 (s, 4H), 2.31 |
| | (q, 2H), 1.67 (s, 2H), 1.38-1.21 (m, 12H). |
| 76 | (3S,4R)-3-fluoro-1-(4-((8-((S)-2-(fluoromethyl)azetidin-1-yl)-5-isopropyl-2,7- |
| | naphthyridin-3-yl)amino)pyrimidin-2-yl)-3-methylpiperidin-4-ol |
| | 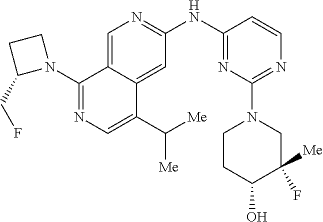 [SMILES: CC(C)c1cnc(N2CC[C@H]2CF)c2cnc(Nc3ccnc(N4CC[C@@H](O)[C@@](C)(F)C4)n3)cc12] |
| | RCl: (S)-6-chloro-1-(2-(fluoromethyl)azetidin-1-yl)-4-isopropyl-2,7-naphthyridine |
| | (Preparation 172); Amine-1; BrettPhos Pd G3, 100° C. |
| | HPLC-18, Gradient (% organic): 25-50% |
| | White solid (14 mg, 5%). LCMS m/z = 484 [M + H]⁺; ¹HNMR (400 MHz, DMSO- |
| | d₆) δ: 10.07 (s, 1H), 9.04 (s, 1H), 8.47 (s, 1H), 8.00 (s, 1H), 7.96 (s, 1H), 6.49 |
| | (s, 1H), 4.87-4.55 (m, 7H), 4.18-4.15 (m, 1H), 3.60-3.55 (m, 1H), 3.33-3.31 |
| | (m, 1H), 3.15-3.09 (m, 2H), 2.45-2.43 (m, 2H), 1.72 (s, 2H), 1.51-1.42 (m, |
| | 9H). |
| 77 | (3S,4R)-3-fluoro-1-(4-((8-(3-(2-hydroxyethyl)azetidin-1-yl)-5-isopropyl-2,7- |
| | naphthyridin-3-yl)amino)pyrimidin-2-yl)-3-methylpiperidin-4-ol |
| | 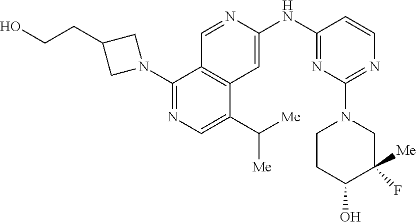 [SMILES: CC(C)c1cnc(N2CC(CCO)C2)c2cnc(Nc3ccnc(N4CC[C@@H](O)[C@@](C)(F)C4)n3)cc12] |
| | RCl: 2-(1-(6-chloro-4-isopropyl-2,7-naphthyridin-1-yl)azetidin-3-yl)ethan-1-ol |
| | (Preparation 182); Amine-1; RuPhos Pd G3, 100° C. |
| | HPLC-19, Gradient (% organic): 8-18% |
| | White solid (20 mg, 15%). LCMS m/z = 496 [M + H]⁺; ¹HNMR (300 MHz, DMSO- |
| | d₆) δ: 10.03 (s, 1H), 9.02 (s, 1H), 8.44 (s, 1H), 8.01 (d, 1H), 7.94 (s, 1H), 6.49 |
| | (d, 1H), 5.02 (d, 1H), 4.79-4.59 (m, 2H), 4.51-4.39 (m, 3H), 4.07-3.96 (m, |
| | 2H), 3.64-3.39 (m, 3H), 3.23-3.02 (m, 3H), 2.84 (p, 1H), 1.79 (q, 2H), 1.71 (s, |
| | 2H), 1.38 (s, 1H), 1.34-1.24 (m, 8H). |
| 78 | (3S,4R)-3-fluoro-1-(4-((8-((2R,3S)-3-hydroxy-2,3-dimethylazetidin-1-yl)-5- |
| | isopropyl-2,7-naphthyridin-3-yl)amino)pyrimidin-2-yl)-3-methylpiperidin-4-ol |
| | 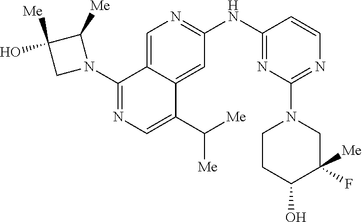 [SMILES: CC(C)c1cnc(N2C[C@](C)(O)[C@H]2C)c2cnc(Nc3ccnc(N4CC[C@@H](O)[C@@](C)(F)C4)n3)cc12] |
| | RCl: (2R,3S)-1-(6-chloro-4-isopropyl-2,7-naphthyridin-1-yl)-2,3-dimethylazetidin- |
| | 3-ol (Preparation 197); Amine-1; BrettPhos Pd G3, 100° C. |
| | HPLC-2: Gradient (% organic): 28-40%) |
| | White solid (64 mg, 16%). LCMS m/z = 496 [M + H]⁺; ¹HNMR (400 MHz, DMSO- |
| | d₆) δ: 10.09 (s, 1H), 9.03 (s, 1H), 8.50 (s, 1H), 8.07-7.92 (m, 2H), 6.48 (d, |
| | 1H), 5.40 (s, 1H), 5.04 (d, 1H), 4.80-4.54 (m, 2H), 4.54-4.36 (m, 2H), 3.89 (d, |
| | 1H), 3.62-3.46 (m, 1H), 3.14 (q, 2H), 1.74 (d, 2H), 1.48-1.21 (m, 5H). |
| 79 | ((1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3- |
| | yl)methyl)dimethylphosphine oxide |
| | 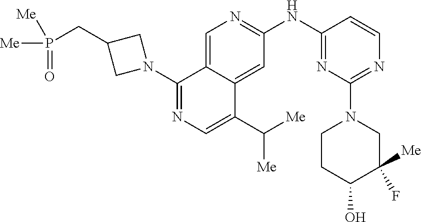 [SMILES: CC(C)c1cnc(N2CC(CP(C)(C)=O)C2)c2cnc(Nc3ccnc(N4CC[C@@H](O)[C@@](C)(F)C4)n3)cc12] |
| | RCl: ((1-(6-chloro-4-isopropyl-2,7-naphthyridin-1-yl)azetidin-3- |
| | yl)methyl)dimethylphosphine oxide (Preparation 164); Amine-1; RuPhos Pd |
| | G3, 130° C. |
| | Prep-TLC (20:1 DCM/MeOH); HPLC-2: Gradient (% organic): 27-47%) |
| | Yellow solid (15.9 mg, 41%). LCMS m/z = 541 [M + H]⁺; ¹HNMR (300 MHz, |
| | DMSO-d₆) δ: 10.04 (s, 1H), 9.01 (s, 1H), 8.45 (s, 1H), 8.05-7.92 (m, 2H), |
| | 6.50 (d, 1H), 5.02 (d, 1H), 4.65-4.49 (m,4H), 4.16-4.05 (m, 2H), 3.31- |
| | 3.09(m,5H),2.11 (dd, 2H), 1.70-1.30 (m, 17H). |
| 80 | 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-N-methylazetidine-3- |
| | carboxamide |
| | 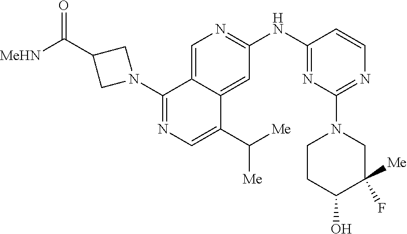 [SMILES: CNC(=O)C1CN(c2ncc(C(C)C)c3cc(Nc4ccnc(N5CC[C@@H](O)[C@@](C)(F)C5)n4)ncc23)C1] |
| | RCl: 1-(6-chloro-4-isopropyl-2,7-naphthyridin-1-yl)-N-methylazetidine-3- |
| | carboxamide (Preparation 245); Amine-1 |
| | BrettPhos Pd G3, 100° C. |
| | HPLC-6: Gradient (% organic): 20-38%) |
| | Yellow solid (26 mg, 20%). LCMS m/z = 509 [M + H]⁺; ¹HNMR (300 MHz, |
| | DMSO-d₆) δ: 10.06 (s, 1H), 9.01 (s, 1H), 8.46 (s, 1H), 8.10-7.84 (m, 3H), |
| | 6.48 (d, 1H), 5.02 (d, 1H), 4.79-4.55 (m, 2H), 4.41 (dt, 4H,), 3.64-3.40 (m, |
| | 2H), 3.22-2.94 (m, 3H), 2.61 (d, 3H), 1.86-1.62 (m, 2H), 1.47-1.19 (m, 9H). |
| 81 | (3S,4R)-3-fluoro-1-(4-((8-((2R,3S)-3-hydroxy-2-methylpyrrolidin-1-yl)-5- |
| | isopropyl-2,7-naphthyridin-3-yl)amino)pyrimidin-2-yl)-3-methylpiperidin-4-ol |
| | 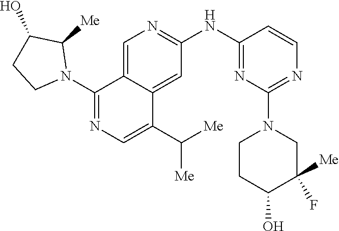 [SMILES: CC(C)c1cnc(N2CC[C@H](O)[C@H]2C)c2cnc(Nc3ccnc(N4CC[C@@H](O)[C@@](C)(F)C4)n3)cc12] |
| | RCl: (2R,3S)-1-(6-chloro-4-isopropyl-2,7-naphthyridin-1-yl)-2-methylpyrrolidin-3- |
| | ol (Preparation 211); Amine-1; XPhos Pd G4, 100° C. |
| | LCMS m/z = 496 [M + H]⁺; ¹HNMR (500 MHz, DMSO-d₆) δ: 9.97 (d, 1H), 9.18 (d, |
| | 1H), 8.37 (s, 1H), 8.00-7.82 (m, 2H), 6.42 (d, 1H), 5.00 (dd, 1H), 4.95 (d, |
| | 1H), 4.72-4.54 (m, 1H), 4.14 (p, 1H), 4.02 (q, 1H), 3.91 (p, 1H), 3.64 (td, 1H), |
| | 3.55-3.40 (m, 1H), 3.24 (s, 2H), 3.08 (q, 2H), 2.09 (dq, 1H), 1.77-1.55 (m, |
| | 3H), 1.33-1.22 (m, 9H), 1.18 (d, 3H). |
| 82 | methyl 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-1,6-diazaspiro[3.3]heptane-6- |
| | carboxylate |
| | 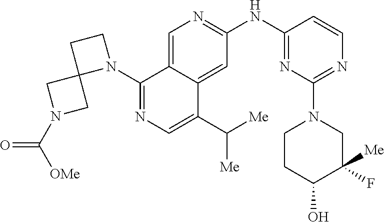 [SMILES: COC(=O)N1CC2(CCN2c2ncc(C(C)C)c3cc(Nc4ccnc(N5CC[C@@H](O)[C@@](C)(F)C5)n4)ncc23)C1] |
| | RCl: methyl 1-(6-chloro-4-isopropyl-2,7-naphthyridin-1-yl)-1,6- |
| | diazaspiro[3.3]heptane-6-carboxylate (Preparation 239); Amine-1; XPhos Pd |
| | G4, 100° C. |
| 83 | N-((1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3- |
| | yl)methyl)methanesulfonamide |
| | 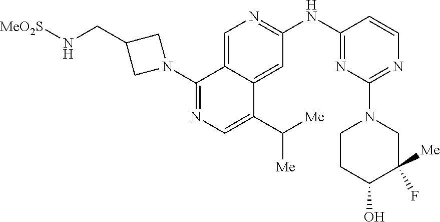 [SMILES: CC(C)c1cnc(N2CC(CNS(C)(=O)=O)C2)c2cnc(Nc3ccnc(N4CC[C@@H](O)[C@@](C)(F)C4)n3)cc12] |
| | RCl: N-((1-(6-chloro-4-isopropyl-2,7-naphthyridin-1-yl)azetidin-3- |
| | yl)methyl)methanesulfonamide (Preparation 227); Amine-1; XPhos Pd G4, |
| | 100° C. |
| | LCMS m/z = 599 [M + H]⁺; ¹HNMR (500 MHz, DMSO-d₆) δ: 9.98 (s, 1H), 8.96 (s, |
| | 1H), 8.40 (s, 1H), 7.96 (d, 1H), 7.91 (s, 1H), 7.16 (t, 1H), 6.44 (d, 1H), 4.95 |
| | (d, 1H), 4.74-4.43 (m, 2H), 4.35 (t, 2H), 4.03 (t, 2H), 3.60-3.41 (m, 1H), 3.19 |
| | (t, 2H), 3.06 (t, 2H), 1.75-1.59 (m, 2H), 1.37-1.21 (m, 9H). |
| 84 | methyl ((1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3-yl)methyl)carbamate |
| | 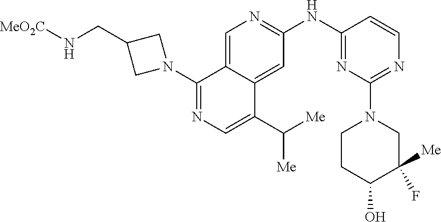 [SMILES: COC(=O)NCC1CN(c2ncc(C(C)C)c3cc(Nc4ccnc(N5CC[C@@H](O)[C@@](C)(F)C5)n4)ncc23)C1] |
| | RCl: methyl ((1-(6-chloro-4-isopropyl-2,7-naphthyridin-1-yl)azetidin-3- |
| | yl)methyl)carbamate (Preparation 235); Amine-1; XPhos Pd G4, 100° C. |
| | LCMS m/z = 539 [M + H]⁺; ¹HNMR (500 MHz, DMSO-d₆) δ: 9.98 (s, 1H), 8.95 (s, |
| | 1H), 8.39 (s, 1H), 7.30 (t, 1H), 6.44 (d, 1H), 4.95 (d, 1H), 4.76-4.47 (m, 2H), |
| | 4.39-4.24 (m, 2H), 4.05-3.90 (m, 2H), 3.47 (s, 4H), 3.06 (t, 2H), 2.86-2.71 (m, |
| | 1H), 1.66 (s, 2H), 1.35-1.20 (m, 9H). |
| 85 | 3-fluoro-1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-N-methylazetidine-3- |
| | carboxamide |
| | 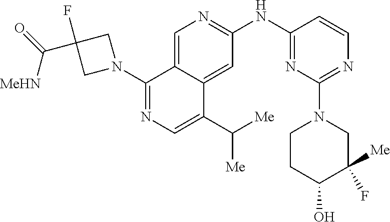 [SMILES: CNC(=O)C1(F)CN(c2ncc(C(C)C)c3cc(Nc4ccnc(N5CC[C@@H](O)[C@@](C)(F)C5)n4)ncc23)C1] |
| | RCl: 1-(6-chloro-4-isopropyl-2,7-naphthyridin-1-yl)-3-fluoro-N-methylazetidine-3- |
| | carboxamide (Preparation 225); Amine-1; XPhos Pd G4, 90° C. |
| | RP-ISCO (0-30% MeCN/H2O (+0.1% TFA) followed by neutralisation with 1M |
| | NaOH |
| | Off-white foam (15.2 mg, 35%). LCMS m/z = 527 [M + H]⁺; ¹HNMR (400 MHz, |
| | DMSO-d₆) δ: 10.07 (s, 1H), 9.03 (s, 1H), 8.49 (s, 1H), 8.43-8.33 (m, 1H), |
| | 8.08-7.96 (m, 2H), 6.49 (d, 1H), 4.99 (d, 1H), 4.81-4.50 (m, 6H), 3.63-3.46 |
| | (m, 1H), 3.40-3.29 (m, 1H), 3.21-3.06 (m, 2H), 2.67 (d, 3H), 1.78-1.64 (m, |
| | 2H), 1.40-1.27 (m, 9H). |
| 86 | 2-(1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3-yl)-N,N- |
| | dimethylacetamide |
| | 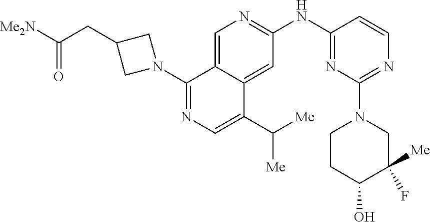 [SMILES: CC(C)c1cnc(N2CC(CC(=O)N(C)C)C2)c2cnc(Nc3ccnc(N4CC[C@@H](O)[C@@](C)(F)C4)n3)cc12] |
| | RCl: 2-(1-(6-chloro-4-isopropyl-2,7-naphthyridin-1-yl)azetidin-3-yl)-N,N- |
| | dimethylacetamide (Preparation 246); Amine-1; RuPhos Pd G3, 100° C. |
| | HPLC-18, Gradient (% organic): 26-39% |
| | Off-white solid, 17.6 mg, 19%. LCMS m/z = 537 [M + H]⁺; ¹HNMR (400 MHz, |
| | DMSO-d₆) δ: 10.09 (s, 1H), 9.03 (s, 1H), 8.03 (d, 1H), 7.97 (s, 1H), 6.49 (d, |
| | 1H), 5.05 (d, 1H), 4.87-4.60 (m, 2H), 4.50 (t, 2H), 4.02 (dd, 2H), 3.68-3.49 |
| | (m, 1H), 3.32-3.28 (m, 1H), 3.23-3.01 (m, 3H), 2.97 (s, 3H), 2.83 (s, 3H), |
| | 2.78 (d, 2H), 1.80-1.60 (m, 2H), 1.44-1.28 (m, 9H). |
| 87 | 2-(1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3-yl)-N- |
| | methylacetamide |
| | 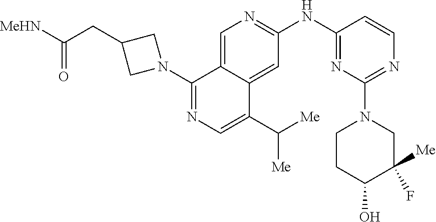 [SMILES: CNC(=O)CC1CN(c2ncc(C(C)C)c3cc(Nc4ccnc(N5CC[C@@H](O)[C@@](C)(F)C5)n4)ncc23)C1] |
| | RCl: 2-(1-(6-chloro-4-isopropyl-2,7-naphthyridin-1-yl)azetidin-3-yl)-N- |
| | methylacetamide (Preparation 247); Amine-1; RuPhos Pd G3, 100° C. |
| | HPLC-18, Gradient (% organic): 22-35% |
| | Yield: 2.4 mg, 3%. LCMS m/z = 523 [M + H]⁺; ¹HNMR (400 MHz, DMSO-d₆) δ: |
| | 10.08 (s, 1H), 9.03 (s, 1H), 8.48 (s, 1H), 8.03 (d, 1H), 7.97 (s, 1H), 7.85 (d, |
| | 1H), 6.50 (d, 1H), 5.05 (s, 1H), 4.79-4.57 (m, 2H), 4.48 (t, 2H), 4.05 (t, 2H), |
| | 3.56 (d, 1H), 3.32-2.99 (m, 5H), 2.58 (d, 3H), 1.82-1.64 (m, 2H), 1.43-1.27 |
| | (m, 9H). |
| 88 | (3S,4R)-1-(4-((8-(3-(1H-1,2,4-triazol-1-yl)azetidin-1-yl)-5-isopropyl-2,7- |
| | naphthyridin-3-yl)amino)pyrimidin-2-yl)-3-fluoro-3-methylpiperidin-4-ol |
| | 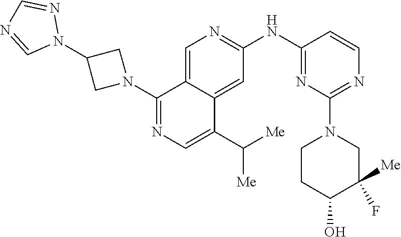 [SMILES: CC(C)c1cnc(N2CC(n3cncn3)C2)c2cnc(Nc3ccnc(N4CC[C@@H](O)[C@@](C)(F)C4)n3)cc12] |
| | RCl: 1-(3-(1H-1,2,4-triazol-1-yl)azetidin-1-yl)-6-chloro-4-isopropyl-2,7- |
| | naphthyridine (Preparation 176); Amine-1; RuPhos Pd G3, 100° C. |
| | HPLC-2: Gradient (% organic): 20-45%) |
| | Yield: 17.8 mg, 19%. LCMS m/z = 519 [M + H]⁺; ¹HNMR (300 MHz, DMSO-d₆) δ: |
| | 10.11 (s, 1H), 9.08 (s, 1H), 8.76 (s, 1H), 8.52 (s, 1H), 8.10 (s, 1H), 8.08-8.00 |
| | (m, 2H), 6.51 (d, 1H), 5.60 (s, 1H), 5.05 (d, 1H), 4.88 (t, 2H), 4.81-4.61 (m, |
| | 4H), 3.67-3.45 (m, 1H), 3.25-3.07 (m, 2H), 1.75 (s, 2H), 1.41 (s, 1H), 1.34 |
| | (m, 8H). |
| 89 | (3S,4R)-1-(4-((8-(3-(1H-1,2,3-triazol-1-yl)azetidin-1-yl)-5-isopropyl-2,7- |
| | naphthyridin-3-yl)amino)pyrimidin-2-yl)-3-fluoro-3-methylpiperidin-4-ol |
| | 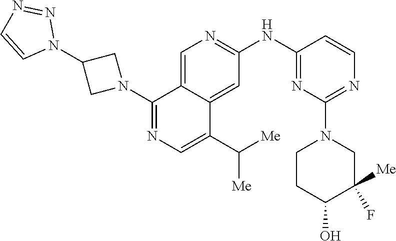 [SMILES: CC(C)c1cnc(N2CC(n3ccnn3)C2)c2cnc(Nc3ccnc(N4CC[C@@H](O)[C@@](C)(F)C4)n3)cc12] |
| | RCl: 1-(3-(1H-1,2,3-triazol-1-yl)azetidin-1-yl)-6-chloro-4-isopropyl-2,7- |
| | naphthyridine (Preparation 165); Amine-1; RuPhos Pd G3, 100° C. |
| | Prep-TLC (20:1 DCM/MeOH); HPLC-23, Gradient (% organic): 23-35% |
| | White solid (10 mg, 9%). LCMS m/z = 519 [M + H]⁺; ¹HNMR (400 MHz, DMSO- |
| | d₆) δ: 10.12 (s, 1H), 9.11 (s, 1H), 8.53 (s, 1H), 8.49 (d, 1H), 8.04 (d, 2H), 7.85 |
| | (d, 1H), 6.51 (d, 1H), 5.76 (s, 1H), 5.05 (d, 1H), 4.96 (t, 1H), 4.72 (dd, 4H), |
| | 3.62 (s, 1H), 3.54 (s, 2H), 1.41 (s, 2H), 1.34 (dd, 10H). |
| 90 | (3S,4R)-3-fluoro-1-(4-((5-isopropyl-8-(1-methyl-4,6-dihydropyrrolo[3,4-c]pyrazol- |
| | 5(1H)-yl)-2,7-naphthyridin-3-yl)amino)pyrimidin-2-yl)-3-methylpiperidin-4-ol |
| | 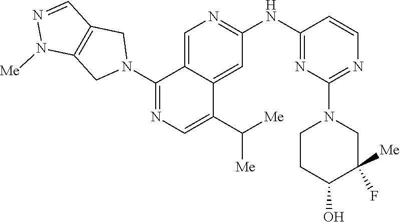 [SMILES: CC(C)c1cnc(N2Cc3cnn(C)c3C2)c2cnc(Nc3ccnc(N4CC[C@@H](O)[C@@](C)(F)C4)n3)cc12] |
| | RCl: 6-chloro-4-isopropyl-1-(1-methyl-4,6-dihydropyrrolo[3,4-c]pyrazol-5(1H)-yl)- |
| | 2,7-naphthyridine (Preparation 167); Amine-1; BrettPhos Pd G3, 100° C. |
| | Prep-TLC (20:1 DCM/MeOH); HPLC-23, Gradient (% organic): 30-40% |
| | Off-white solid (52 mg, 37%); LCMS m/z = 518 [M + H]⁺; ¹HNMR (400 MHz, |
| | DMSO-d₆) δ: 10.04 (s, 1H), 9.48 (s, 1H), 8.45 (s, 1H), 8.08-7.90 (m, 2H), |
| | 7.27 (s, 1H), 6.54 (d, 1H), 5.15-4.89 (m, 5H), 4.79-4.52 (m, 2H), 3.83 (s, 3H), |
| | 3.53 (dt, 1H), 3.24-2.97 (m, 2H), 1.83-1.59 (m, 2H), 1.45-1.26 (m, 9H). |
| 91 | 1-(1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3-yl)pyrrolidin-2-one |
| | 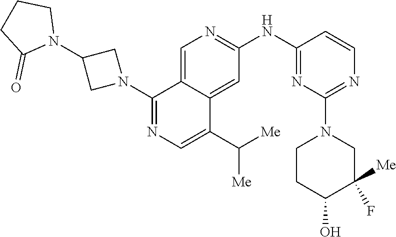 [SMILES: CC(C)c1cnc(N2CC(N3CCCC3=O)C2)c2cnc(Nc3ccnc(N4CC[C@@H](O)[C@@](C)(F)C4)n3)cc12] |
| | RCl: 1-(1-(6-chloro-4-isopropyl-2,7-naphthyridin-1-yl)azetidin-3-yl)pyrrolidin-2- |
| | one (Preparation 166); Amine-1; BrettPhos Pd G3, 100° C. |
| | Prep-TLC (20:1 DCM/MeOH); HPLC-23, Gradient (% organic): 20-45% |
| | Off-white solid: 28 mg, 30%; LCMS m/z = 535 [M + H]⁺; ¹HNMR (300 MHz, |
| | DMSO-d₆) δ: 10.06 (s, 1H), 9.06 (s, 1H), 8.47 (s, 1H), 8.01 (d, 1H), 7.98 (s, |
| | 1H), 6.48 (d, 1H), 5.13-4.94 (m, 2H), 4.80-4.32 (m, 6H), 3.60 (t, 2H), 3.25- |
| | 2.89 (m, 4H), 2.27 (t, 2H), 1.96 (t, 2H), 1.85-1.65 (m, 2H), 1.49-1.20 (m, 9H). |
| 92 | 3-fluoro-1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-N,N-dimethylazetidine-3- |
| | carboxamide |
| | 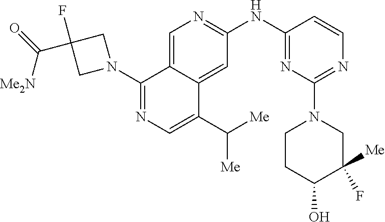 [SMILES: CC(C)c1cnc(N2CC(F)(C(=O)N(C)C)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-3-fluoro-N,N- |
| | dimethylazetidine-3-carboxamide (Preparation 202); Amine-1; XPhos Pd G4, |
| | 90° C. |
| | RP-ISCO (0-35% MeCN/H2O (+0.1% TFA) followed by neutralisation with 1M |
| | NaOH |
| | Pale yellow foam (42 mg, 46%). LCMS m/z = 541 [M + H]⁺; ¹HNMR (500 MHz, |
| | DMSO-d₆) δ: 10.12 (s, 1H), 9.08 (s, 1H), 8.51 (s, 1H), 8.09-7.96 (m, 2H), |
| | 6.52 (d, 1H), 5.03 (d, 1H), 5.00-4.87 (m, 2H), 4.77-4.61 (m, 4H), 3.64-3.49 |
| | (m, 1H), 3.22-3.07 (m, 2H), 3.00 (d, 3H), 2.90 (d, 3H), 1.80-1.66 (m, 2H), |
| | 1.40-1.28 (m, 9H). |
| 93 | 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-N,N,3-trimethylazetidine-3- |
| | carboxamide |
| | 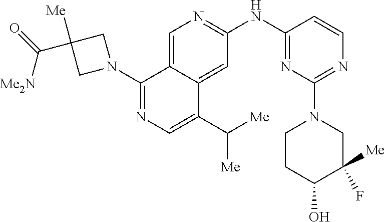 [SMILES: CC(C)c1cnc(N2CC(C)(C(=O)N(C)C)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-N,N,3-trimethylazetidine-3- |
| | carboxamide (Preparation 201); Amine-1; XPhos Pd G4, 90° C. |
| | RP-ISCO (0-30% MeCN/H2O (+0.1% TFA) followed by neutralisation with 1M |
| | NaOH |
| | White solid (35 mg, 65%). LCMS m/z = 538 [M + H]⁺; ¹HNMR (400 MHz, DMSO- |
| | d₆) δ: 10.05 (s, 1H), 9.05 (s, 1H), 8.46 (s, 1H), 8.01 (d, 1H), 7.95 (s, 1H), 6.50 |
| | (d, 1H), 5.00 (d, 1H), 4.78-4.60 (m, 2H), 4.60-4.54 (m, 2H), 4.20 (dd, 2H), |
| | 3.62-3.45 (m, 1H), 3.22-3.04 (m, 3H), 2.86 (s, 3H), 2.81 (s, 3H), 1.77-1.66 |
| | (m, 2H), 1.61 (s, 3H), 1.39-1.27 (m, 9H). |
| 94 | ((1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)azetidin-3-yl)imino)dimethyl-l6- |
| | sulfanone |
| | 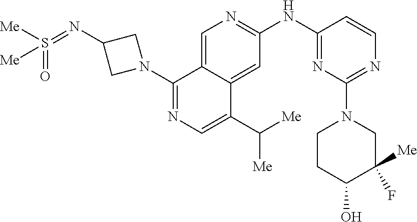 [SMILES: CC(C)c1cnc(N2CC(N=S(C)(C)=O)C2)c2cnc(Nc3ccnc(N4CC[C@@H](O)[C@@](C)(F)C4)n3)cc12] |
| | RCl: ((1-(6-chloro-4-isopropyl-2,7-naphthyridin-1-yl)azetidin-3-yl)imino)dimethyl- |
| | l6-sulfanone (Preparation 194); Amine-1; BrettPhos Pd G3, 100° C. |
| | HPLC-18, Gradient (% organic): 20-35% |
| | Yield: 6 mg, 22%. LCMS m/z = 543 [M + H]⁺; ¹HNMR (300 MHz, DMSO-d₆) δ: |
| | 10.03 (s, 1H), 9.00 (s, 1H), 8.44 (s, 1H), 8.01 (d, 1H), 7.94 (s, 1H), 6.49 (d, |
| | 1H), 5.03 (d, 1H), 4.78-4.56 (m, 4H), 4.39 (p, 1H), 4.12-4.02 (m, 2H), 3.65- |
| | 3.44 (m, 1H), 3.28-3.03 (m, 8H), 1.72 (d, 2H), 1.43-1.22 (m, 9H). |
| 95 | (3S,4R)-3-fluoro-1-(4-((8-((2R,3S)-3-hydroxy-2-methylazetidin-1-yl)-5-isopropyl- |
| | 2,7-naphthyridin-3-yl)amino)pyrimidin-2-yl)-3-methylpiperidin-4-ol |
| | 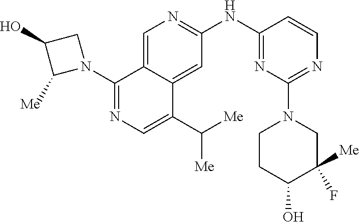 [SMILES: CC(C)c1cnc(N2C[C@H](O)[C@H]2C)c2cnc(Nc3ccnc(N4CC[C@@H](O)[C@@](C)(F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-1; BrettPhos Pd G3, 100° C. |
| | HPLC-12, Gradient (% organic): 30-57% |
| | Pale yellow solid (15 mg, 9%). LCMS m/z = 482 [M + H]⁺; ¹HNMR (400 MHz, |
| | DMSO-d₆) δ: 10.06 (s, 1H), 9.12 (s, 1H), 8.79(s, 1H), 8.01 (d, 1H), 7.74 (s, |
| | 1H), 6.41 (d, 1H), 5.64 (d, 1H),5.01(d,1H), 4.73 (t, 3H), 4.15 (ddd, 2H), 3.89 |
| | (s, 1H), 3.79 (s, 2H), 3.27-3.06 (m, 2H), 1.64 (d, 2H), 1.41 (d, 6H), 1.30 (dd, |
| | 6H). |
| 96 | dimethyl ((1-(6-((2-((3S,4R)-3-fluoro-4-hydroxy-3-methylpiperidin-1-yl)pyrimidin- |
| | 4-yl)amino)-4-isopropyl-2,7-naphthyridin-1-yl)azetidin-3- |
| | yl)methyl)phosphonate |
| | 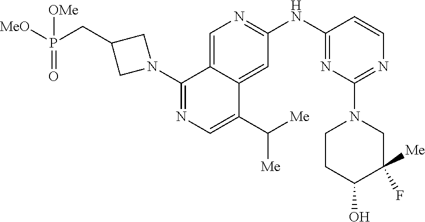 [SMILES: COP(=O)(CC1CN(c2ncc(C(C)C)c3cc(Nc4ccnc(N5CC[C@@H](O)[C@@](C)(F)C5)n4)ncc23)C1)OC] |
| | RCl: dimethyl ((1-(6-chloro-4-isopropyl-2,7-naphthyridin-1-yl)azetidin-3- |
| | yl)methyl)phosphonate (Preparation 229); Amine-1; BrettPhos Pd G3, 100° C. |
| | HPLC-28, Gradient (% organic): 27-37% |
| | Pale yellow solid (23 mg, 31%). LCMS m/z = 574 [M + H]⁺; ¹HNMR (400 MHz, |
| | DMSO-d₆) δ: 10.07 (s, 1H), 9.02 (s, 1H), 8.47 (s, 1H), 8.03 (d, 1H), 7.97 (s, |
| | 1H), 6.50 (d, 1H), 5.08 (d, 1H),4.84-4.60 (m, 2H), 4.51 (t, 2H), 4.14-4.05 (m, |
| | 2H), 3.66 (d, 6H), 3.62-3.43 (m, 1H), 3.54 (ddd, 1H), 3.44 (d, 1H), 3.31 (p, |
| | 1H), 3.18-2.97 (m, 3H), 2.24 (dd, 2H), 1.73 (s, 2H), 1.49-1.21 (m, 9H). |
| 97 | 1-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-N,N-dimethylazetidine-3- |
| | carboxamide |
| | 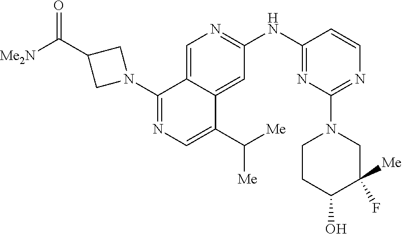 [SMILES: CC(C)c1cnc(N2CC(C(=O)N(C)C)C2)c2cnc(Nc3ccnc(N4CC[C@@H](O)[C@@](C)(F)C4)n3)cc12] |
| | RCl: 1-(6-chloro-4-isopropyl-2,7-naphthyridin-1-yl)-N,N-dimethylazetidine-3- |
| | carboxamide (Preparation 160); Amine-1; BrettPhos Pd G3, 100° C. |
| | HPLC-24, Gradient (% organic): 24-38% |
| | Pale yellow solid (41 mg, 29%). LCMS m/z = 523 [M + H]⁺; ¹HNMR (300 MHz, |
| | DMSO-d₆) δ: 10.09 (s, 1H), 9.11 (s, 1H), 8.70 (s, 1H), 8.00 (d, 1H), 7.60 (s, |
| | 1H), 6.44 (d, 1H), 5.04 (d, 1H), 4.91-4.58 (m, 2H), 4.46 (t, 2H), 4.27 (t, 2H), |
| | 3.93 (q, 1H), 3.79-3.45 (m, 2H), 3.12 (dd, 2H), 2.92 (s, 3H), 2.85 (s, 3H), 1.73 |
| | (s, 2H), 1.49-1.21 (m, 9H). |
| 98 | 6-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-1-thia-6-azaspiro[3.3]heptane |
| | 1,1-dioxide |
| | 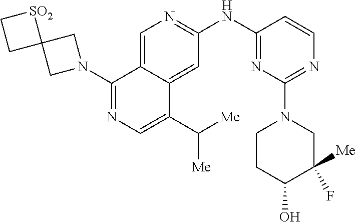 [SMILES: CC(C)c1cnc(N2CC3(CCS3(=O)=O)C2)c2cnc(Nc3ccnc(N4CC[C@@H](O)[C@@](C)(F)C4)n3)cc12] |
| | RCl: 6-(6-chloro-4-isopropyl-2,7-naphthyridin-1-yl)-1-thia-6-azaspiro[3.3]heptane |
| | 1,1-dioxide (Preparation 217); Amine-1; XPhos Pd G4, 90° C. |
| | Triturate with H₂O followed by MeOH. |
| | Tan solid (76 mg, 66%). LCMS m/z = 542 [M + H]⁺; ¹HNMR (400 MHz, DMSO-d₆) |
| | δ: 10.14 (s, 1H), 9.03 (s, 1H), 8.52 (s, 1H), 8.07-7.97 (m, 2H), 6.48 (d, 1H), |
| | 5.01 (d, 1H), 4.80-4.56 (m, 6H), 4.19-4.04 (m, 2H), 3.62-3.46 (m, 1H), 3.38- |
| | 3.31 (m, 1H), 3.22-3.05 (m, 2H), 2.46-2.40 (m, 2H), 1.73 (d, 2H), 1.41-1.27 |
| | (m, 9H). |
| 99 | 2-(6-((2-((3S,4R)-3-fluoro-4-hydroxy-3-methylpiperidin-1-yl)pyrimidin-4- |
| | yl)amino)-4-isopropyl-2,7-naphthyridin-1-yl)-6-methyl-2,6- |
| | diazaspiro[3.4]octan-5-one |
| | 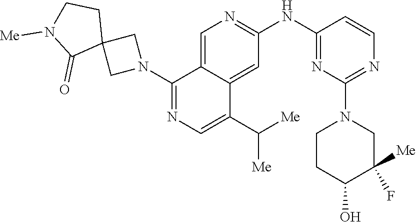 [SMILES: CC(C)c1cnc(N2CC3(CCN(C)C3=O)C2)c2cnc(Nc3ccnc(N4CC[C@@H](O)[C@@](C)(F)C4)n3)cc12] |
| | RCl: 2-(6-chloro-4-isopropyl-2,7-naphthyridin-1-yl)-6-methyl-2,6- |
| | diazaspiro[3.4]octan-5-one (Preparation 175); Amine-1; BrettPhos Pd G3, |
| | 100° C. |
| | HPLC-24, Gradient (% organic): 30-40% |
| | Pale yellow solid (60 mg, 30%). LCMS m/z = 535 [M + H]⁺; ¹HNMR (300 MHz, |
| | DMSO-d₆) δ: 10.09 (s, 1H), 9.01 (s, 1H), 8.49 (s, 1H), 8.06-7.94 (m, 2H), |
| | 6.48 (d, 1H), 5.08 (d, 1H), 4.80-4.60 (m, 2H), 4.43 (d, 2H), 4.33-4.24 (m, |
| | 2H), 3.32 (t, 3H), 3.11 (dd, 2H), 2.77 (s, 3H), 2.42 (t, 2H), 1.72 (q, 2H), 1.39 |
| | (s, 2H), 1.35-1.26 (m, 8H). |
| 100 | (3S,4R)-3-fluoro-1-(4-((5-isopropyl-8-(3- |
| | (((trifluoromethyl)sulfinyl)methyl)azetidin-1-yl)-2,7-naphthyridin-3- |
| | yl)amino)pyrimidin-2-yl)-3-methylpiperidin-4-ol |
| | 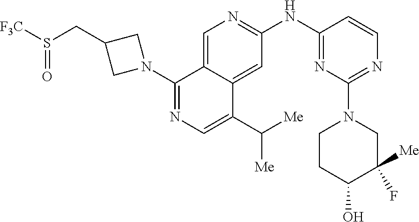 [SMILES: CC(C)c1cnc(N2CC(CS(=O)C(F)(F)F)C2)c2cnc(Nc3ccnc(N4CC[C@@H](O)[C@@](C)(F)C4)n3)cc12] |
| | RCl: 6-chloro-4-isopropyl-1-(3-(((trifluoromethyl)sulfinyl)methyl)azetidin-1-yl)- |
| | 2,7-naphthyridine (Preparation 189); Amine-1; BrettPhos Pd G3, 130° C. |
| | HPLC-12, Gradient (% organic): 30-57% |
| | White solid (5 mg, 6%). LCMS m/z = 582 [M + H]⁺; ¹HNMR (300 MHz, DMSO-d₆) |
| | δ: 10.08 (s, 1H), 9.01 (s, 1H), 8.47 (s, 1H), 8.11-7.90 (m, 2H), 6.48 (d, 1H), |
| | 5.04 (d, 1H), 4.64 (ddd, 4H), 4.27 (dt, 2H), 3.63 (d, 2H), 3.56-3.41 (m, 1H), |
| | 3.20-2.95 (m, 2H), 1.86-1.51 (m, 2H), 1.44-1.21 (m, 9H). |
| 101 | (3S,4R)-3-fluoro-1-(4-((5-isopropyl-8-(3-(oxetan-3-yl)azetidin-1-yl)-2,7- |
| | naphthyridin-3-yl)amino)pyrimidin-2-yl)-3-methylpiperidin-4-ol |
| | 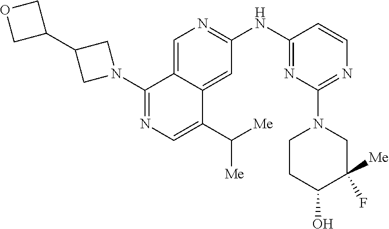 [SMILES: CC(C)c1cnc(N2CC(C3COC3)C2)c2cnc(Nc3ccnc(N4CC[C@@H](O)[C@@](C)(F)C4)n3)cc12] |
| | RCl: 6-chloro-4-isopropyl-1-(3-(oxetan-3-yl)azetidin-1-yl)-2,7-naphthyridine |
| | (Preparation 203); Amine-1; XPhos Pd G4, 90° C. |
| | SiO2, ISCO (0-5% MeOH/DCM) |
| | Pale yellow solid (26 mg, 52%). LCMS m/z = 508 [M + H]⁺; ¹HNMR (400 MHz, |
| | CDCl₃) δ: 10.08 (s, 1H), 9.07 (s, 1H), 8.49 (s, 1H), 8.05 (d, 1H), 8.00 (s, 1H), |
| | 6.53 (d, 1H), 5.04 (d, 1H), 4.75 (dd, 2H), 4.68 (d, 1H), 4.59-4.47 (m, 2H), |
| | 4.38 (t, 2H), 4.13 (dd, 2H), 3.69-3.48 (m, 1H), 3.15 (d, 1H), 1.75 (s, 2H), |
| | 1.42-1.31 (m, 9H). Some protons are under the residual solvent peak. |
| 102 | (3S,4R)-1-(4-((5-cyclopropyl-8-((2R,3S)-3-hydroxy-2-methylazetidin-1-yl)-2,7- |
| | naphthyridin-3-yl)amino)pyrimidin-2-yl)-3-fluoro-3-methylpiperidin-4-ol |
| | 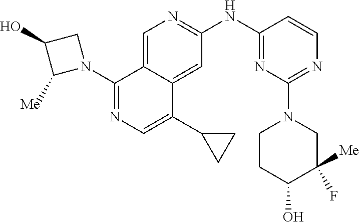 [SMILES: C[C@@H]1[C@@H](O)CN1c1ncc(C2CC2)c2cc(Nc3ccnc(N4CC[C@@H](O)[C@@](C)(F)C4)n3)ncc12] |
| | RCl: (2R,3S)-1-(6-chloro-4-cyclopropyl-2,7-naphthyridin-1-yl)-2-methylazetidin-3- |
| | ol (Preparation 317); Amine-1; XPhos Pd G4, 90° C. |
| | RP-ISCO (0-30% MeCN/H2O (+0.1% TFA) followed by neutralisation with |
| | NaHCO₃ |
| | Yellow solid (8.7 mg, 20%). LCMS m/z = 480 [M + H]⁺; ¹HNMR (400 MHz, |
| | DMSO-d₆) δ: 10.06 (s, 1H), 9.03 (s, 1H), 8.56 (s, 1H), 8.02 (d, 1H), 7.83 (s, |
| | 1H), 6.51 (d, 1H), 5.59 (d, 1H), 4.97 (d, 1H), 4.85 (t, 1H), 4.77-4.57 (m, 2H), |
| | 4.38 (s, 1H), 4.15 (p, 1H), 3.77 (s, 1H), 3.58-3.41 (m, 1H), 3.20-3.01 (m, 2H), |
| | 1.92 (ddd, 1H), 1.63 (d, 2H), 1.39 (d, 3H), 1.30 (d, 3H), 0.96-0.86 (m, 2H), |
| | 0.71-0.58 (m, 2H). |
| 103 | (3S,4R)-3-fluoro-1-(4-((8-((2R,3S)-3-hydroxy-2-methylazetidin-1-yl)-5- |
| | isopropylisoquinolin-3-yl)amino)pyrimidin-2-yl)-3-methylpiperidin-4-ol |
| | 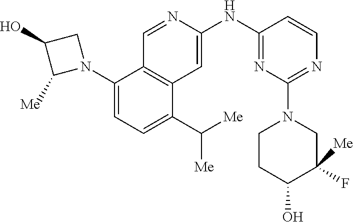 [SMILES: CC(C)c1ccc(N2C[C@H](O)[C@H]2C)c2cnc(Nc3ccnc(N4CC[C@@H](O)[C@@](C)(F)C4)n3)cc12] |
| | RCl: (2R,3S)-1-(3-chloro-5-isopropylisoquinolin-8-yl)-2-methylazetidin-3-ol |
| | (Preparation 123); Amine-1; BrettPhos Pd G3, 100° C. |
| | HPLC-23, Gradient (% organic): 34-44% |
| | White solid (105 mg, 30%). LCMS m/z = 481 [M + H]⁺; ¹HNMR (400 MHz, |
| | DMSO-d₆) δ: 9.90 (s, 1H), 9.06 (s, 1H), 8.62 (s, 1H), 7.99 (d, 1H), 7.43 (d, |
| | 1H), 6.60 (d, 1H), 6.45 (d, 1H), 5.62 (d, 1H), 5.05 (d, 1H), 4.92-4.45 (m, 3H), |
| | 4.16 (p, 1H), 4.00 (t, 1H), 3.53 (m, 2H), 3.43 (t, 1H), 3.10-3.20 (m, 2H), 1.74 |
| | (s, 2H), 1.34 (ddd, 12H). |
| 104 | (3S,4R)-3-fluoro-1-(4-((5-isopropyl-8-(3-(5-methyl-1,3,4-oxadiazol-2-yl)azetidin-1- |
| | yl)isoquinolin-3-yl)amino)pyrimidin-2-yl)-3-methylpiperidin-4-ol |
| | 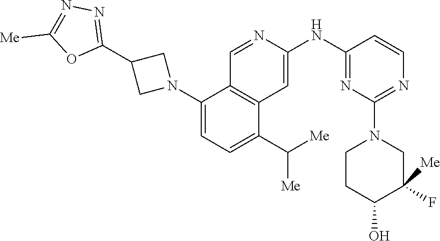 [SMILES: Cc1nnc(C2CN(c3ccc(C(C)C)c4cc(Nc5ccnc(N6CC[C@@H](O)[C@@](C)(F)C6)n5)ncc34)C2)o1] |
| | RCl: 2-(1-(3-chloro-5-isopropylisoquinolin-8-yl)azetidin-3-yl)-5-methyl-1,3,4- |
| | oxadiazole (Preparation 124); Amine-1; RuPhos Pd G3, 100° C. |
| | HPLC-23, Gradient (% organic): 34-44% |
| | White solid (30 mg, 27%). LCMS m/z = 533 [M + H]⁺; ¹HNMR (300 MHz, DMSO- |
| | d₆) δ: 9.88 (s, 1H), 9.05 (s, 1H), 8.62 (s, 1H), 7.98 (d, 1H), 7.43 (d, 1H), 6.48 |
| | (dd, 2H), 5.04 (d, 1H), 4.82-4.64 (m, 2H), 4.59 (dt, 2H), 4.38-4.22 (m, 3H), |
| | 3.66-3.45 (m, 1H), 3.51 (s, 1H), 3.18 (d, 1H), 3.10 (t, 1H), 2.50 (d, 2H), 1.77- |
| | 1.69 (m, 2H), 1.40 (s, 1H), 1.36-1.23 (m, 7H). |
| 105 | (3S,4R)-3-fluoro-1-(4-((8-((S)-2-(hydroxymethyl)azetidin-1-yl)-5- |
| | isopropylisoquinolin-3-yl)amino)pyrimidin-2-yl)-3-methylpiperidin-4-ol |
| | 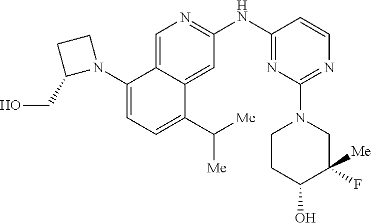 [SMILES: CC(C)c1ccc(N2CC[C@H]2CO)c2cnc(Nc3ccnc(N4CC[C@@H](O)[C@@](C)(F)C4)n3)cc12] |
| | RCl: (S)-(1-(3-chloro-5-isopropylisoquinolin-8-yl)azetidin-2-yl)methanol |
| | (Preparation 121); Amine-1; BrettPhos Pd G3, 100° C. |
| | Prep-TLC (20:1 DCM/MeOH) |
| | HPLC-23, Gradient (% organic): 33-43% |
| | Yellow solid (7.9 mg, 10%). LCMS m/z = 481 [M + H]⁺; ¹HNMR (400 MHz, |
| | DMSO-d₆) δ: 9.85 (s, 1H), 9.05 (s, 1H), 8.60 (s, 1H), 7.98 (d, 1H), 7.40 (d, |
| | 1H), 6.67 (d, 1H), 6.45 (d, 1H), 5.03 (d, 1H), 4.89-4.60 (m, 3H), 4.46-4.38 |
| | (m, 2H), 3.78 (q, 1H), 3.65 (t, 2H), 3.60-3.45 (m, 2H), 3.27-3.02 (m, 2H), |
| | 2.37-2.19 (m, 1H), 1.82-1.65 (m, 2H), 1.45-1.24 (m, 9H). |
| 106 | (3S,4R)-3-fluoro-1-(4-((5-isopropyl-8-((R)-2-methylazetidin-1-yl)-2,6- |
| | naphthyridin-3-yl)amino)pyrimidin-2-yl)-3-methylpiperidin-4-ol |
| | 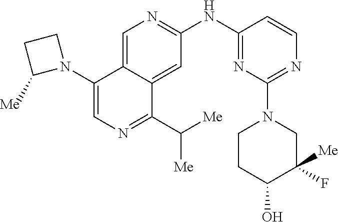 [SMILES: CC(C)c1ncc(N2CC[C@H]2C)c2cnc(Nc3ccnc(N4CC[C@@H](O)[C@@](C)(F)C4)n3)cc12] |
| | RCl: (R)-7-chloro-1-isopropyl-4-(2-methylazetidin-1-yl)-2,6-naphthyridine |
| | (Preparation 346); Amine-1; BrettPhos Pd G3, 100° C. |
| | Prep-TLC (25:1 DCM/MeOH) |
| | HPLC-23, Gradient (% organic): 35-60%) |
| | Yellow solid (14.9 mg, 15%). LCMS m/z = 466 [M + H]⁺; ¹HNMR (300 MHz, |
| | DMSO-d₆) δ: 10.07 (s, 1H), 9.10 (s, 1H), 8.69 (s, 1H), 8.00 (d, 1H), 7.68 (s, |
| | 1H), 6.43 (d, 1H), 5.03 (d, 1H), 4.81-4.61 (m, 2H), 4.58-4.40 (m, 2H), 3.87 |
| | (d, 1H), 3.74-3.43 (m, 1H), 3.11 (s, 2H), 1.73 (s, 3H), 1.38 (d, 5H), 1.34-1.24 |
| | (m, 7H). |
| 107 | (3S,4R)-3-fluoro-1-(4-((5-isopropyl-8-((2R,3S)-3-methoxy-2-methylazetidin-1-yl)- |
| | 2,7-naphthyridin-3-yl)amino)pyrimidin-2-yl)-3-methylpiperidin-4-ol |
| | 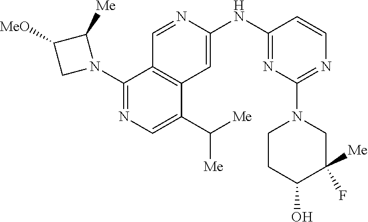 [SMILES: CO[C@H]1CN(c2ncc(C(C)C)c3cc(Nc4ccnc(N5CC[C@@H](O)[C@@](C)(F)C5)n4)ncc23)[C@@H]1C] |
| | RCl: 6-chloro-4-isopropyl-1-((2R,3S)-3-methoxy-2-methylazetidin-1-yl)-2,7- |
| | naphthyridine (Preparation 251); Amine-1; BrettPhos Pd G4, 90° C. |
| | SiO₂, ISCO (0-10% MeOH/DCM) |
| | Yellow solid (78.6 mg, 69%). LCMS m/z = 496 [M + H]⁺; ¹HNMR (400 MHz, |
| | DMSO-d₆) δ: 10.09 (s, 1H), 9.08 (s, 1H), 8.51 (s, 1H), 8.11-7.96 (m, 2H), |
| | 6.53 (d, 1H), 5.05 (d, 1H), 4.89 (dd, 1H), 4.80-4.63 (m, 2H), 4.63-4.48 (m, |
| | 1H), 4.08-3.90 (m, 1H), 3.87 (dd, 1H), 3.64-3.48 (m, 1H), 3.29 (s, 3H), 3.24- |
| | 3.02 (m, 2H), 1.47 (d, 3H), 1.42-1.32 (m, 9H). Multiplet underneath residual |
| | solvent peak. |
| 108 | (3S,4R)-3-fluoro-1-(4-((5-isopropyl-8-((2R,3S)-3-(methoxy-d3)-2-methylazetidin- |
| | 1-yl)-2,7-naphthyridin-3-yl)amino)pyrimidin-2-yl)-3-methylpiperidin-4-ol |
| | 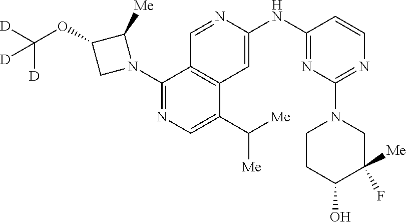 [SMILES: [2H]C([2H])([2H])O[C@H]1CN(c2ncc(C(C)C)c3cc(Nc4ccnc(N5CC[C@@H](O)[C@@](C)(F)C5)n4)ncc23)[C@@H]1C] |
| | RCl: 6-chloro-4-isopropyl-1-((2R,3S)-3-(methoxy-d3)-2-methylazetidin-1-yl)-2,7- |
| | naphthyridine (Preparation 250); Amine-1; BrettPhos Pd G4, 90° C. |
| | SiO₂, ISCO (0-10% MeOH/DCM) |
| | Yellow solid (26.4 mg, 44%). LCMS m/z = 499 [M + H]⁺; ¹HNMR (400 MHz, |
| | DMSO-d₆) δ: 10.09 (s, 1H), 9.08 (s, 1H), 8.51 (s, 1H), 8.14-7.95 (m, 2H), |
| | 6.53 (d, 1H), 5.05 (d, 1H), 4.89 (dd, 1H), 4.80-4.63 (m, 2H), 4.59-4.42 (m, |
| | 1H), 4.01 (q, 1H), 3.93-3.77 (m, 1H), 3.67-3.47 (m, 1H), 3.17 (q, 2H), 1.76 (s, |
| | 2H), 1.47 (d, 3H), 1.43-1.32 (m, 9H). |
| 109 | (3S,4R)-1-(4-((8-((2R,3S)-3-(difluoromethoxy)-2-methylazetidin-1-yl)-5-isopropyl- |
| | 2,7-naphthyridin-3-yl)amino)pyrimidin-2-yl)-3-fluoro-3-methylpiperidin-4-ol |
| | 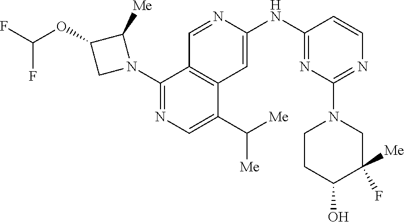 [SMILES: CC(C)c1cnc(N2C[C@H](OC(F)F)[C@H]2C)c2cnc(Nc3ccnc(N4CC[C@@H](O)[C@@](C)(F)C4)n3)cc12] |
| | RCl: 6-chloro-1-((2R,3S)-3-(difluoromethoxy)-2-methylazetidin-1-yl)-4-isopropyl- |
| | 2,7-naphthyridine (Preparation 249); Amine-1; BrettPhos Pd G4, 90° C. |
| | SiO₂, ISCO (0-10% MeOH/DCM) |
| | Off-white solid (5 mg, 21%). LCMS m/z = 499 [M + H]⁺; ¹HNMR (400 MHz, |
| | DMSO-d₆) δ: 10.13 (s, 1H), 9.08 (s, 1H), 8.54 (s, 1H), 8.06 (s, 1H), 6.82 (t, |
| | 1H), 6.53 (d, 1H), 5.02 (dd, 2H), 4.72 (ddd, 4H), 4.03 (dd, 1H), 3.81-3.49 (m, |
| | 1H), 3.41-3.34 (m, 1H), 3.18 (d, 2H), 2.61 (2H), 1.48 (d,), 1.42-1.32 (m, 9H). |
| 110 | (3S,4R)-1-(4-((8-(azetidin-1-yl)-5-isopropylisoquinolin-3-yl)amino)pyrimidin-2-yl)- |
| | 3-fluoro-3-methylpiperidin-4-ol |
| | 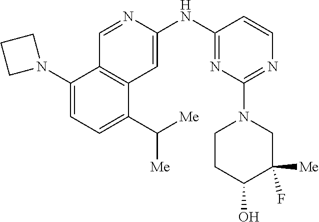 [SMILES: CC(C)c1ccc(N2CCC2)c2cnc(Nc3ccnc(N4CC[C@@H](O)[C@@](C)(F)C4)n3)cc12] |
| | RCl: 8-(azetidin-1-yl)-3-chloro-5-isopropylisoquinoline (Preparation 115); Amine- |
| | 1; BrettPhos Pd G3, 100° C. |
| | HPLC-23, Gradient (% organic): 42-52% |
| | Yellow solid (20 mg, 46%). LCMS m/z = 451 [M + H]⁺; ¹HNMR (300 MHz, |
| | DMSO-d₆) δ: 9.84 (s, 1H), 9.07 (s, 1H), 8.59 (s, 1H), 7.99 (d, 1H), 7.41 (d, |
| | 1H), 6.47 (d, 1H), 6.38 (d, 1H), 5.03 (d, 1H), 4.84-4.54 (m, 2H), 4.15 (t, 4H), |
| | 3.68-3.37 (m, 2H), 3.23-2.98 (m, 2H), 2.44-2.28 (m, 2H), 1.82-1.65 (m, 2H), |
| | 1.49-1.15 (m, 9H). |
| 111 | (3S,4R)-1-(4-((8-(3-(1,3,4-oxadiazol-2-yl)azetidin-1-yl)-5-isopropylisoquinolin-3- |
| | yl)amino)pyrimidin-2-yl)-3-fluoro-3-methylpiperidin-4-ol |
| | 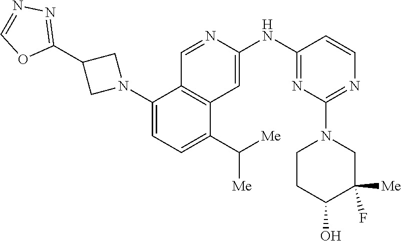 [SMILES: CC(C)c1ccc(N2CC(c3nnco3)C2)c2cnc(Nc3ccnc(N4CC[C@@H](O)[C@@](C)(F)C4)n3)cc12] |
| | RCl: 2-(1-(3-chloro-5-isopropylisoquinolin-8-yl)azetidin-3-yl)-1,3,4-oxadiazole |
| | (Preparation 119); Amine-1; BrettPhos Pd G3, 100° C. |
| | HPLC-23, Gradient (% organic): 35-45% |
| | Yellow solid (30 mg). LCMS m/z = 519 [M + H]⁺; ¹HNMR (300 MHz, MeOH-d₄) δ: |
| | 9.13 (d, 1H), 8.97 (s, 1H), 8.66 (s, 1H), 7.96 (d, 1H), 7.50 (d, 1H), 6.61 (d, |
| | 1H), 6.38 (d, 1H), 4.67 (dt, 4H), 4.47-4.30 (m, 3H), 3.78-3.62 (m, 1H), 3.61 |
| | (q, 1H), 3.31-3.16 (m, 1H), 1.95-1.83 (m, 1H), 1.50 (s, 2H), 1.46-1.34 (m, |
| | 8H). |
| 112 | (3S,4R)-3-fluoro-1-(4-((5-isopropyl-8-(3-(4-methyl-4H-1,2,4-triazol-3-yl)azetidin- |
| | 1-yl)isoquinolin-3-yl)amino)pyrimidin-2-yl)-3-methylpiperidin-4-ol |
| | 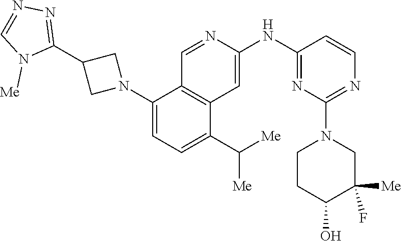 [SMILES: CC(C)c1ccc(N2CC(c3nncn3C)C2)c2cnc(Nc3ccnc(N4CC[C@@H](O)[C@@](C)(F)C4)n3)cc12] |
| | RCl: 3-chloro-5-isopropyl-8-(3-(4-methyl-4H-1,2,4-triazol-3-yl)azetidin-1- |
| | yl)isoquinoline (Preparation 125); Amine-1; BrettPhos Pd G3, 100° C. |
| | HPLC-12, Gradient (% organic): 30-57% |
| | Yellow solid (10 mg, 32%). LCMS m/z = 532 [M + H]⁺; ¹HNMR (400 MHz, |
| | DMSO-d₆) δ: 9.86 (s, 1H), 9.09 (s, 1H), 8.61 (s, 1H), 8.43 (s, 1H), 7.98 (d, |
| | 1H), 7.44 (d, 1H), 6.48 (dd, 2H), 5.02 (d, 1H), 4.82-4.55 (m, 4H), 4.38 (td, |
| | 2H), 4.32-4.12 (m, 1H), 3.69-3.45 (m, 5H), 3.21-3.05 (m, 2H), 1.82-1.61 (m, |
| | 2H), 1.48-1.15 (m, 9H). |
| 113 | (3S,4R)-3-fluoro-1-(4-((5-isopropyl-8-(3-methyl-3-(1,3,4-oxadiazol-2-yl)azetidin-1- |
| | yl)isoquinolin-3-yl)amino)pyrimidin-2-yl)-3-methylpiperidin-4-ol |
| | 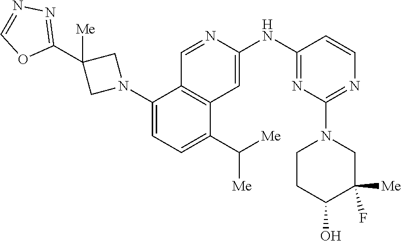 [SMILES: CC(C)c1ccc(N2CC(C)(c3nnco3)C2)c2cnc(Nc3ccnc(N4CC[C@@H](O)[C@@](C)(F)C4)n3)cc12] |
| | RCl: 2-(1-(3-chloro-5-isopropylisoquinolin-8-yl)-3-methylazetidin-3-yl)-1,3,4- |
| | oxadiazole (Preparation 117); Amine-1; BrettPhos Pd G3, 100° C. |
| | HPLC-23, Gradient (% organic): 38-50% |
| | Yellow solid (18 mg, 38%). LCMS m/z = 533 [M + H]⁺; ¹HNMR (300 MHz, |
| | DMSO-d₆) δ: 9.84 (s, 1H), 9.22 (s, 1H), 9.06 (s, 1H), 8.61 (s, 1H), 7.98 (d, |
| | 1H), 7.43 (d, 1H), 6.48 (dd, 2H), 4.99 (d, 1H), 4.83-4.56 (m, 2H), 4.51 (d, |
| | 2H), 4.28 (dd, 2H), 3.56 (dq, 2H), 3.12 (dd, 2H), 1.84 (s, 3H), 1.76-1.65 (m, |
| | 2H), 1.43-1.20 (m, 9H). |
| 114 | (3S,4R)-3-fluoro-1-(4-((1-isopropyl-4-(pyrrolidin-1-yl)pyrido[3,4-d]pyridazin-7- |
| | yl)amino)pyrimidin-2-yl)-3-methylpiperidin-4-ol |
| | 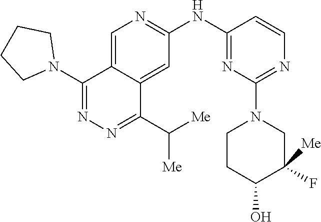 [SMILES: CC(C)c1nnc(N2CCCC2)c2cnc(Nc3ccnc(N4CC[C@@H](O)[C@@](C)(F)C4)n3)cc12] |
| | RCl: 7-chloro-1-isopropyl-4-(pyrrolidin-1-yl)pyrido[3,4-d]pyridazine (Preparation |
| | 368); Amine-1; XPhos Pd G2, 100° C. |
| | HPLC-23, Gradient (% organic): 20-45% |
| | Yellow solid (24.8 mg, 38%). LCMS m/z = 467 [M + H]⁺; ¹HNMR (400 MHz, |
| | MeOH-d₄) δ: 9.41 (s, 1H), 8.58 (s, 1H), 8.04 (d, 1H), 6.45 (d, 1H), 4.77-4.68 |
| | (m, 2H), 3.92-3.88 (m, 4H), 3.73-3.65 (m, 2H), 3.29-3.22 (m, 2H), 2.11-2.08 |
| | (m, 4H), 1.94-1.91 (m, 2H), 1.51-1.44 (m, 9H). |
| 115 | (3S,4R)-1-(4-((8-((S)-2-((dimethylamino)methyl)pyrrolidin-1-yl)-5-isopropyl-2,7- |
| | naphthyridin-3-yl)amino)pyrimidin-2-yl)-3-fluoro-3-methylpiperidin-4-ol |
| | 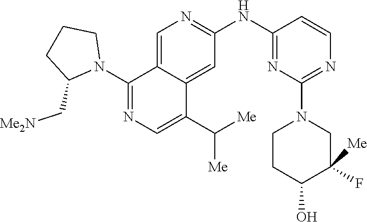 [SMILES: CC(C)c1cnc(N2CCC[C@H]2CN(C)C)c2cnc(Nc3ccnc(N4CC[C@@H](O)[C@@](C)(F)C4)n3)cc12] |
| | RCl: (S)-1-(1-(6-chloro-4-isopropyl-2,7-naphthyridin-1-yl)pyrrolidin-2-yl)-N,N- |
| | dimethylmethanamine (Preparation 169); Amine-1; XPhos Pd G2, 120° C. |
| | HPLC-14, Gradient (% organic): 35-45% |
| | Yellow solid (8.2 mg, 12%). LCMS m/z = 523 [M + H]⁺; ¹HNMR (400 MHz, |
| | MeOH-d₄) δ: 9.24 (s, 1H), 8.51 (s, 1H), 7.99-7.97 (m, 2H), 6.40 (d, 1H), 4.83- |
| | 4.69 (m, 3H), 4.11-4.05 (m, 1H), 3.73-3.63 (m, 2H), 3.48-3.42 (m, 1H), 3.25- |
| | 3.12 (m, 2H), 2.76 (d, 1H), 2.48 (br s, 1H), 2.37 (br s, 6H), 2.33-2.24 (m, 1H), |
| | 2.06 (br s, 1H), 1.94-1.79 (m, 4H), 1.46 (d, 3H,), 1.39 (dd, 6H). |
| 116 | N-(2-(1-oxa-7-azaspiro[3.5]nonan-7-yl)pyrimidin-4-yl)-5-isopropyl-8-(6-methyl- |
| | 1,6-diazaspiro[3.3]heptan-1-yl)-2,7-naphthyridin-3-amine |
| | 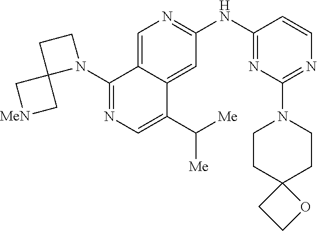 [SMILES: CC(C)c1cnc(N2CCC23CN(C)C3)c2cnc(Nc3ccnc(N4CCC5(CCO5)CC4)n3)cc12] |
| | RCl: 6-chloro-4-isopropyl-1-(6-methyl-1,6-diazaspiro[3.3]heptan-1-yl)-2,7- |
| | naphthyridine (Preparation 237); Amine-2; XPhos Pd G3, 100° C. |
| | HPLC-20, Gradient (% organic): 8-22% |
| | Yellow solid (2.9 mg, 1.8%). LCMS m/z = 535 [M + H]⁺; ¹HNMR (300 MHz, |
| | MeOH-d₄) δ: 9.18 (s, 1H), 8.60 (s, 1H), 8.14 (s, 1H), 8.02 (d, 1H), 6.42 (d, |
| | 1H), 5.02-4.91 (m, 2H), 4.64 (t, 4H), 4.24 (d, 2H), 4.02-3.75 (m, 4H), 3.46- |
| | 3.36 (m, 1H), 3.00 (s, 3H), 2.76 (t, 2H), 2.55 (t, 2H), 1.99 (q, 4H), 1.44 (d, |
| | 6H). |
| 117 | 1-(6-((2-(1-oxa-7-azaspiro[3.5]nonan-7-yl)pyrimidin-4-yl)amino)-4-isopropyl-2,7- |
| | naphthyridin-1-yl)azetidin-3-ol |
| | 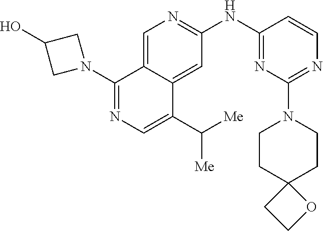 [SMILES: CC(C)c1cnc(N2CC(O)C2)c2cnc(Nc3ccnc(N4CCC5(CCO5)CC4)n3)cc12] |
| | RCl: 1-(6-chloro-4-isopropyl-2,7-naphthyridin-1-yl)azetidin-3-ol (Preparation 161); |
| | Amine-2; BrettP Pd G3, 100° C. |
| | HPLC-18, Gradient (% organic): 28-41% |
| | Yellow solid (21.2 mg, 11%). LCMS m/z = 462 [M + H]⁺; ¹HNMR (300 MHz, |
| | DMSO-d₆) δ: 10.04 (s, 1H), 9.01 (s, 1H), 8.49 (s, 1H), 8.02 (d, 1H), 7.96 (s, |
| | 1H), 6.48 (d, 1H), 5.69 (d, 1H), 4.69-4.51 (m, 3H), 4.44 (t, 2H), 4.08 (dd, 2H), |
| | 3.79 (tt, 4H), 3.26 (q, 1H), 2.43-2.32 (m, 2H), 1.82 (q, 4H), 1.31 (d, 6H). |
| 118 | (3S,4R)-1-(4-((8-(3-fluoroazetidin-1-yl)-5-isopropyl-2,7-naphthyridin-3- |
| | yl)amino)pyrimidin-2-yl)-4-methoxypiperidin-3-ol or (3R,4S)-1-(4-((8-(3- |
| | fluoroazetidin-1-yl)-5-isopropyl-2,7-naphthyridin-3-yl)amino)pyrimidin-2- |
| | yl)-4-methoxypiperidin-3-ol |
| | 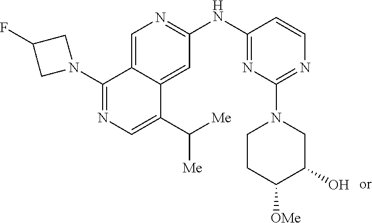 [SMILES: CO[C@@H]1CCN(c2nccc(Nc3cc4c(C(C)C)cnc(N5CC(F)C5)c4cn3)n2)C[C@@H]1O] |
| | 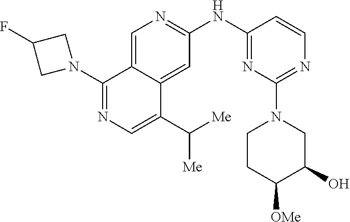 [SMILES: CO[C@H]1CCN(c2nccc(Nc3cc4c(C(C)C)cnc(N5CC(F)C5)c4cn3)n2)C[C@H]1O] |
| | RCl: 6-chloro-1-(3-fluoroazetidin-1-yl)-4-isopropyl-2,7-naphthyridine (Preparation |
| | 278); Amine-3; BrettP Pd G3, 100° C. |
| | HPLC-18, Gradient (% organic): 35-46% |
| | Yellow solid (15.2 mg, 23%). LCMS m/z = 486 [M + H]⁺; ¹HNMR (400 MHz, |
| | DMSO-d₆) δ: 10.06 (s, 1H), 9.03 (s, 1H), 8.57 (s, 1H), 8.05-7.97 (m, 2H), |
| | 6.47 (d, 1H), 5.62 (dq, 1H), 5.47 (tt, 1H), 4.76-4.67 (m, 1H), 4.66 (t, 2H), |
| | 4.51-4.44 (m, 1H), 4.44-4.36 (m, 1H), 3.97 (d, 2H), 3.78 (s, 1H), 3.82-3.65 |
| | (m, 1H), 3.46 (dt, 1H), 3.34 (d, 5H), 1.86 (s, 1H), 1.62 (s, 1H), 1.31 (d, 6H). |
| 119 | (3S,4R)-1-(4-((8-((2R,3S)-3-hydroxy-2-methylazetidin-1-yl)-5-isopropyl-2,6- |
| | naphthyridin-3-yl)amino)pyrimidin-2-yl)-4-methoxypiperidin-3-ol or |
| | (3R,4S)-1-(4-((8-((2R,3S)-3-hydroxy-2-methylazetidin-1-yl)-5-isopropyl-2,6- |
| | naphthyridin-3-yl)amino)pyrimidin-2-yl)-4-methoxypiperidin-3-ol |
| | 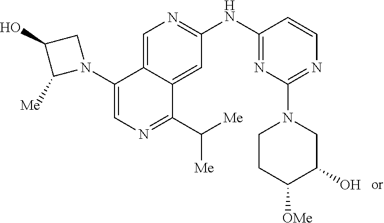 [SMILES: CO[C@@H]1CCN(c2nccc(Nc3cc4c(C(C)C)ncc(N5C[C@H](O)[C@H]5C)c4cn3)n2)C[C@@H]1O] |
| | 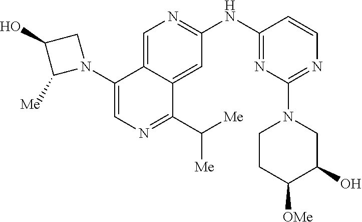 [SMILES: CO[C@H]1CCN(c2nccc(Nc3cc4c(C(C)C)ncc(N5C[C@H](O)[C@H]5C)c4cn3)n2)C[C@H]1O] |
| | RCl: (2R,3S)-1-(7-chloro-1-isopropyl-2,6-naphthyridin-4-yl)-2-methylazetidin-3-ol |
| | (Preparation 348); Amine-3; BrettP Pd G3, 100° C. |
| | HPLC-12, Gradient (% organic): 30-57% |
| | Yellow solid (15 mg, 9%). LCMS m/z = 480 [M + H]⁺; ¹HNMR (400 MHz, DMSO- |
| | d₆) δ: 10.06 (s, 1H), 9.12 (s, 1H), 8.79 (s, 1H), 8.01 (d, 1H), 7.74 (s, 1H), 6.41 |
| | (d, 1H), 5.64 (d, 1H), 4.73 (t, 1H), 4.63 (d, 1H), 4.15 (ddd, 2H), 3.89 (s, 4H), |
| | 3.79 (s, 2H), 3.77-3.66 (m, 1H), 3.64 (s, 1H), 3.59-3.51 (m, 1H), 3.47 (d, 3H), |
| | 1.91-1.85 (m, 1H), 1.64 (d, 1H), 1.41 (d, 3H), 1.30 (dd, 6H). |
| 120 | 1-(6-((2-((3S,4R)-3-hydroxy-4-methoxypiperidin-1-yl)pyrimidin-4-yl)amino)-4- |
| | isopropyl-2,7-naphthyridin-1-yl)-N,N-dimethylazetidine-3-carboxamide or 1- |
| | (6-((2-((3R,4S)-3-hydroxy-4-methoxypiperidin-1-yl)pyrimidin-4-yl)amino)- |
| | 4-isopropyl-2,7-naphthyridin-1-yl)-N,N-dimethylazetidine-3-carboxamide |
| | 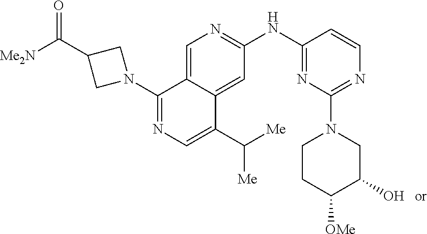 [SMILES: CO[C@@H]1CCN(c2nccc(Nc3cc4c(C(C)C)cnc(N5CC(C(=O)N(C)C)C5)c4cn3)n2)C[C@@H]1O] |
| | 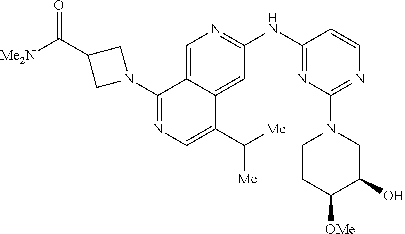 [SMILES: CO[C@H]1CCN(c2nccc(Nc3cc4c(C(C)C)cnc(N5CC(C(=O)N(C)C)C5)c4cn3)n2)C[C@H]1O] |
| | RCl: 1-(6-chloro-4-isopropyl-2,7-naphthyridin-1-yl)-N,N-dimethylazetidine-3- |
| | carboxamide (Preparation 160); Amine-3; BrettP Pd G3, 100° C. |
| | HPLC-18, Gradient (% organic): 27-37% |
| | Yellow solid (35.5 mg, 21%). LCMS m/z = 521 [M + H]⁺; ¹HNMR (300 MHz, |
| | DMSO-d₆) δ: 10.03 (s, 1H), 9.01 (s, 1H), 8.54 (s, 1H), 8.04-7.91 (m, 2H), |
| | 6.44 (d, 1H), 4.64 (d, 1H), 4.53 (t, 2H), 4.40 (dd, 2H), 3.99-3.80 (m, 3H), 3.76 |
| | (s, 3H), 3.67 (dd, 1H), 3.34 (s, 4H), 2.91 (s, 3H), 2.84 (s, 3H), 1.85 (dd, 1H), |
| | 1.66-1.53 (m, 1H), 1.29 (d, 6H). |
| 121 | 1-(7-((2-((3S,4R)-3-hydroxy-4-methoxypiperidin-1-yl)pyrimidin-4-yl)amino)-1- |
| | isopropyl-2,6-naphthyridin-4-yl)-N,N-dimethylazetidine-3-carboxamide or 1- |
| | (7-((2-((3R,4S)-3-hydroxy-4-methoxypiperidin-1-yl)pyrimidin-4-yl)amino)- |
| | 1-isopropyl-2,6-naphthyridin-4-yl)-N,N-dimethylazetidine-3-carboxamide |
| | 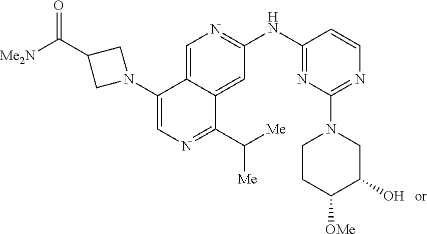 [SMILES: CO[C@@H]1CCN(c2nccc(Nc3cc4c(C(C)C)ncc(N5CC(C(=O)N(C)C)C5)c4cn3)n2)C[C@@H]1O] |
| | 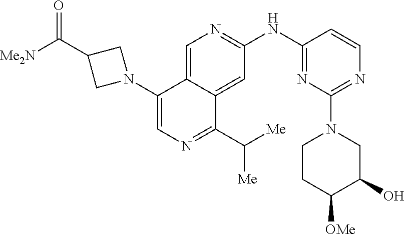 [SMILES: CO[C@H]1CCN(c2nccc(Nc3cc4c(C(C)C)ncc(N5CC(C(=O)N(C)C)C5)c4cn3)n2)C[C@H]1O] |
| | RCl: 1-(7-chloro-1-isopropyl-2,6-naphthyridin-4-yl)-N,N-dimethylazetidine-3- |
| | carboxamide (Preparation 345); Amine-3; BrettP Pd G3, 100° C. |
| | HPLC-18, Gradient (% organic): 24-37% |
| | Yellow solid (30 mg, 24%). LCMS m/z = 521 [M + H]⁺; ¹HNMR (300 MHz, |
| | DMSO-d₆) δ: 10.05 (s, 1H), 9.10 (s, 1H), 8.77 (s, 1H), 7.99 (d, 1H), 7.59 (s, |
| | 1H), 6.40 (d, 1H), 4.64 (d, 1H), 4.45 (t, 2H), 4.27 (t, 2H), 3.93 (dd, 3H), 3.82- |
| | 3.50 (m, 4H), 3.44 (d, 1H), 3.34 (d, 3H), 2.92 (s, 3H), 2.85 (s, 3H), 1.86 (d, |
| | 1H), 1.62 (s, 1H), 1.28 (d, 6H). |
| 122 | (3S,4R)-1-(4-((8-((2R,3S)-3-hydroxy-2-methylazetidin-1-yl)-5-isopropyl-2,7- |
| | naphthyridin-3-yl)amino)pyrimidin-2-yl)-4-methoxypiperidin-3-ol or |
| | (3R,4S)-1-(4-((8-((2R,3S)-3-hydroxy-2-methylazetidin-1-yl)-5-isopropyl-2,7- |
| | naphthyridin-3-yl)amino)pyrimidin-2-yl)-4-methoxypiperidin-3-ol |
| | 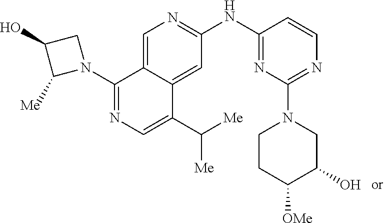 [SMILES: CO[C@@H]1CCN(c2nccc(Nc3cc4c(C(C)C)cnc(N5C[C@H](O)[C@H]5C)c4cn3)n2)C[C@@H]1O] |
| | 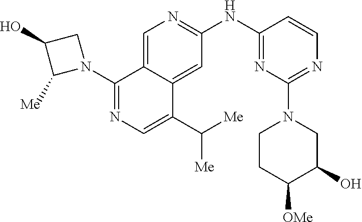 [SMILES: CO[C@H]1CCN(c2nccc(Nc3cc4c(C(C)C)cnc(N5C[C@H](O)[C@H]5C)c4cn3)n2)C[C@H]1O] |
| | RCl: (2R,3S)-1-(6-chloro-4-isopropyl-2,7-naphthyridin-1-yl)-2-methylazetidin-3-ol |
| | (Preparation 213); Amine-3; RuPhos Pd G3, 100° C. |
| | HPLC-19, Gradient (% organic): 24-37% |
| | Yellow solid (30 mg). LCMS m/z = 480 [M + H]⁺; ¹HNMR (300 MHz, DMSO-d₆) |
| | δ: 10.03 (s, 1H), 9.04 (s, 1H), 8.56 (s, 1H), 8.05-7.95 (m, 2H), 6.44 (d, 1H), |
| | 5.65 (dd, 1H), 4.84 (t, 1H), 4.66 (d, 1H), 4.39 (p, 1H), 4.16 (p, 1H), 3.97 (t, |
| | 2H), 3.77 (dt, 2H), 3.67 (dd, 1H), 3.35 (s, 6H), 1.90-1.80 (m, 1H), 1.68-1.56 |
| | (m, 1H), 1.41 (d, 3H), 1.35 (s, 1H), 1.30 (dd, 6H). |
| 123 | (3S,4R)-1-(4-((8-((2R,3S)-3-hydroxy-2-methylazetidin-1-yl)-5- |
| | isopropylisoquinolin-3-yl)amino)pyrimidin-2-yl)-4-methoxypiperidin-3-ol-ol |
| | or (3R,4S)-1-(4-((8-((2R,3S)-3-hydroxy-2-methylazetidin-1-yl)-5- |
| | isopropylisoquinolin-3-yl)amino)pyrimidin-2-yl)-4-methoxypiperidin-3-ol |
| | 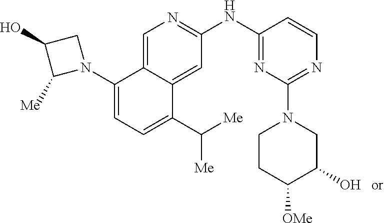 [SMILES: CO[C@@H]1CCN(c2nccc(Nc3cc4c(C(C)C)ccc(N5C[C@H](O)[C@H]5C)c4cn3)n2)C[C@@H]1O] |
| | 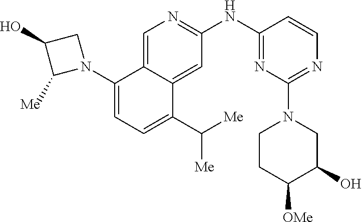 [SMILES: CO[C@H]1CCN(c2nccc(Nc3cc4c(C(C)C)ccc(N5C[C@H](O)[C@H]5C)c4cn3)n2)C[C@H]1O] |
| | RCl: (2R,3S)-1-(3-chloro-5-isopropylisoquinolin-8-yl)-2-methylazetidin-3-ol |
| | (Preparation 123); Amine-3; Allyl BrettPhos Pd OTf, 100° C. |
| | HPLC-23, Gradient (% organic): 30-45% |
| | Yellow solid (25 mg, 30%). LCMS m/z = 479 [M + H]⁺; ¹HNMR (300 MHz, |
| | DMSO-d₆) δ: 9.84 (s, 1H), 9.06 (s, 1H), 8.68 (s, 1H), 7.98 (d, 1H), 7.42 (d, |
| | 1H), 6.59 (d, 1H), 6.42 (d, 1H), 5.60 (d, 1H), 4.83-4.62 (m, 2H), 4.16 (p, 1H), |
| | 4.04-3.87 (m, 3H), 3.81-3.41 (m, 4H), 3.37 (s, 3H), 1.88 (d, 1H), 1.64 (s, 1H), |
| | 1.41-1.21 (m, 9H). |
| 124 | 6-(6-((2-((3S,4R)-3-hydroxy-4-methoxypiperidin-1-yl)pyrimidin-4-yl)amino)-4- |
| | isopropyl-2,7-naphthyridin-1-yl)-1-thia-6-azaspiro[3.3]heptane 1,1-dioxide or |
| | 6-(6-((2-((3R,4S)-3-hydroxy-4-methoxypiperidin-1-yl)pyrimidin-4- |
| | yl)amino)-4-isopropyl-2,7-naphthyridin-1-yl)-1-thia-6-azaspiro[3.3]heptane |
| | 1,1-dioxide |
| | 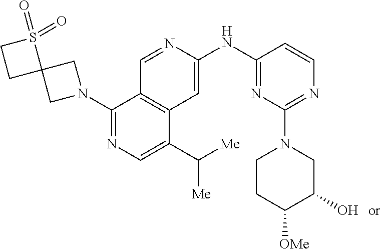 [SMILES: CO[C@@H]1CCN(c2nccc(Nc3cc4c(C(C)C)cnc(N5CC6(CCS6(=O)=O)C5)c4cn3)n2)C[C@@H]1O] |
| | 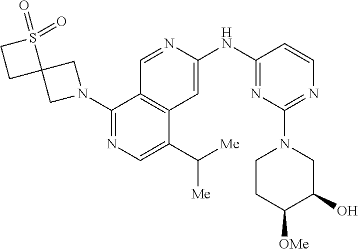 [SMILES: CO[C@H]1CCN(c2nccc(Nc3cc4c(C(C)C)cnc(N5CC6(CCS6(=O)=O)C5)c4cn3)n2)C[C@H]1O] |
| | RCl: 6-(6-chloro-4-isopropyl-2,7-naphthyridin-1-yl)-1-thia-6-azaspiro[3.3]heptane |
| | 1,1-dioxide (Preparation 217); Amine-3; RuPhos Pd G3, 100° C. |
| | HPLC-23, Gradient (% organic): 34-44% |
| | White solid (40 mg, 26%). LCMS m/z = 540 [M + H]⁺; ¹HNMR (300 MHz, DMSO- |
| | d₆) δ: 10.16 (s, 1H), 9.05 (s, 1H), 8.62 (s, 1H), 8.10-7.90 (m, 2H), 6.45 (d, |
| | 1H), 4.78 (d, 2H), 4.72-4.57 (m, 3H), 4.33-4.09 (m, 2H), 3.98 (d, 2H), 3.83- |
| | 3.54 (m, 3H), 3.46 (dt, 1H), 3.39 (d, 4H), 2.49-2.40 (m, 2H), 1.88 (q, 1H), |
| | 1.70-1.52 (m, 1H), 1.32 (d, 6H). |
| 125 | 6-(3-((2-((3S,4R)-3-hydroxy-4-methoxypiperidin-1-yl)pyrimidin-4-yl)amino)-5- |
| | isopropylisoquinolin-8-yl)-1-thia-6-azaspiro[3.3]heptane 1,1-dioxide or 6-(3- |
| | ((2-((3R,4S)-3-hydroxy-4-methoxypiperidin-1-yl)pyrimidin-4-yl)amino)-5- |
| | isopropylisoquinolin-8-yl)-1-thia-6-azaspiro[3.3]heptane 1,1-dioxide |
| | 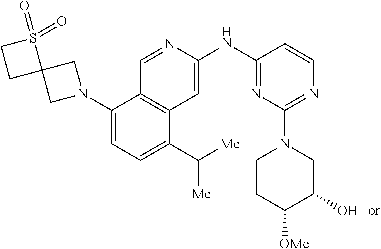 [SMILES: CO[C@@H]1CCN(c2nccc(Nc3cc4c(C(C)C)ccc(N5CC6(CCS6(=O)=O)C5)c4cn3)n2)C[C@@H]1O] |
| | 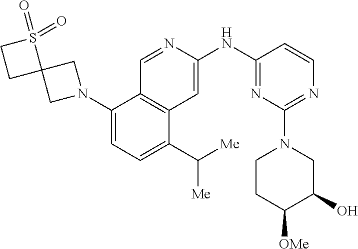 [SMILES: CO[C@H]1CCN(c2nccc(Nc3cc4c(C(C)C)ccc(N5CC6(CCS6(=O)=O)C5)c4cn3)n2)C[C@H]1O] |
| | RCl: 6-(3-chloro-5-isopropylisoquinolin-8-yl)-1-thia-6-azaspiro[3.3]heptane 1,1- |
| | dioxide (Preparation 126); Amine-3; CPhos and Pd₂(dba)₃.CHCl₃, 90° C. |
| | HPLC-23, Gradient (% organic): 50-60% |
| | Yellow solid (3.3 mg, 7%). LCMS m/z = 539 [M + H]⁺; ¹HNMR (300 MHz, DMSO- |
| | d₆) δ: 9.92 (s, 1H), 9.01 (s, 1H), 8.73 (s, 1H), 7.99 (d, 1H), 7.45 (d, 1H), 6.55 |
| | (d, 1H), 6.42 (d, 1H), 4.66-4.58 (m, 3H), 4.36 (d, 2H), 4.20-4.11 (m, 2H), |
| | 3.99 (s, 2H), 3.79 (s, 1H), 3.72 (d, 1H), 3.48 (s, 1H), 3.35 (m, 3H), 3.31 (s, |
| | 1H), 2.48-2.39 (m, 2H), 1.88 (s, 1H), 1.63 (s, 1H), 1.31 (d, 6H). |
| 126 | (3S,4R)-1-(4-((8-(3-(1H-1,2,3-triazol-1-yl)azetidin-1-yl)-5-isopropylisoquinolin-3- |
| | yl)amino)pyridin-2-yl)-4-methoxypiperidin-3-ol or (3R,4S)-1-(4-((8-(3-(1H- |
| | 1,2,3-triazol-1-yl)azetidin-1-yl)-5-isopropylisoquinolin-3-yl)amino)pyridin-2- |
| | yl)-4-methoxypiperidin-3-ol |
| | 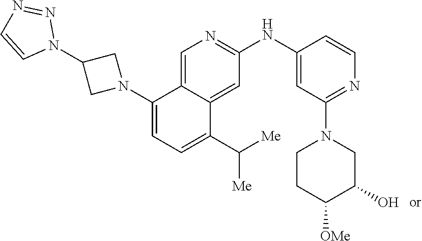 [SMILES: CO[C@@H]1CCN(c2cc(Nc3cc4c(C(C)C)ccc(N5CC(n6ccnn6)C5)c4cn3)ccn2)C[C@@H]1O] |
| | 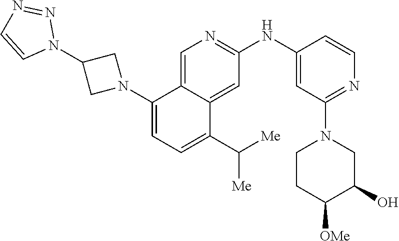 [SMILES: CO[C@H]1CCN(c2cc(Nc3cc4c(C(C)C)ccc(N5CC(n6ccnn6)C5)c4cn3)ccn2)C[C@H]1O] |
| | RCl: 8-(3-(1H-1,2,3-triazol-1-yl)azetidin-1-yl)-3-chloro-5-isopropylisoquinoline |
| | (Preparation 122); Amine-3; RuPhos Pd G3, 100° C. |
| | Prep-TLC (25:1 DCM/MeOH); HPLC-18, Gradient (% organic): 30-50% |
| | Yellow solid (45 mg, 48%). LCMS m/z = 515 [M + H]⁺; ¹HNMR (300 MHz, |
| | DMSO-d₆) δ: 9.89 (s, 1H), 9.11 (s, 1H), 8.68 (s, 1H), 8.49 (d, 1H), 8.00 (d, |
| | 1H), 7.84 (s, 1H), 7.46 (d, 1H), 6.56 (d, 1H), 6.46 (d, 1H), 5.73 (d, 1H), 5.10 |
| | (d, 1H),4.74 (t, 2H), 4.49 (t, 2H), 4.45-4.31 (m, 2H), 3.60-3.49 (m, 1H), 3.20- |
| | 3.07 (m, 4H), 2.05 (s, 3H), 1.39 (s, 1H), 1.32 (dd, 7H). |
| 127 | (3S,4S)-1-(4-((8-(3-(1H-1,2,3-triazol-1-yl)azetidin-1-yl)-5-isopropylisoquinolin-3- |
| | yl)amino)pyridin-2-yl)-4-methoxypiperidin-3-ol or (3R,4R)-1-(4-((8-(3-(1H- |
| | 1,2,3-triazol-1-yl)azetidin-1-yl)-5-isopropylisoquinolin-3-yl)amino)pyridin-2- |
| | yl)-4-methoxypiperidin-3-ol |
| | 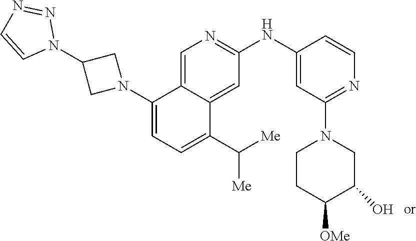 [SMILES: CO[C@H]1CCN(c2cc(Nc3cc4c(C(C)C)ccc(N5CC(n6ccnn6)C5)c4cn3)ccn2)C[C@@H]1O] |
| | 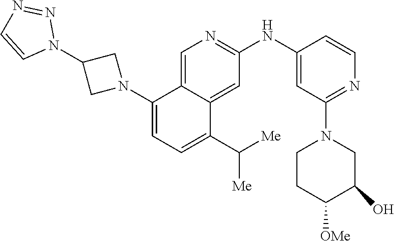 [SMILES: CO[C@@H]1CCN(c2cc(Nc3cc4c(C(C)C)ccc(N5CC(n6ccnn6)C5)c4cn3)ccn2)C[C@H]1O] |
| | RCl: 8-(3-(1H-1,2,3-triazol-1-yl)azetidin-1-yl)-3-chloro-5-isopropylisoquinoline |
| | (Preparation 122); Amine-4; RuPhos Pd G3, 100° C. |
| | Prep-TLC (25:1 DCM/MeOH); HPLC-18, Gradient (% organic): 30-50% |
| | Yellow solid (38 mg, 40%). LCMS m/z = 515 [M + H]⁺; ¹HNMR (300 MHz, |
| | DMSO-d₆) δ: 9.89 (s, 1H), 9.11 (s, 1H), 8.68 (s, 1H), 8.49 (d, 1H), 8.00 (d, |
| | 1H), 7.84 (s, 1H), 7.46 (d, 1H), 6.56 (d, 1H), 6.46 (d, 1H), 5.73 (d, 1H), 5.10 |
| | (d, 1H),4.74 (t, 2H), 4.49 (t, 2H), 4.45-4.31 (m, 2H), 3.60-3.49 (m, 1H), 3.20- |
| | 3.07 (m, 4H), 2.05 (s, 3H), 1.39 (s, 1H), 1.32 (dd, 7H). |
| 128 | 1-(4-((8-((2R,3S)-3-hydroxy-2-methylazetidin-1-yl)-5-isopropyl-2,7-naphthyridin- |
| | 3-yl)amino)pyrimidin-2-yl)-4-methylpiperidin-4-ol |
| | 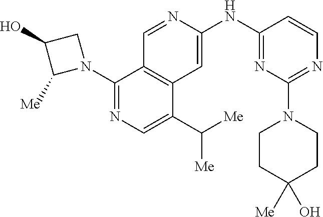 [SMILES: CC(C)c1cnc(N2C[C@H](O)[C@H]2C)c2cnc(Nc3ccnc(N4CCC(C)(O)CC4)n3)cc12] |
| | RCl: (2R,3S)-1-(6-chloro-4-isopropyl-2,7-naphthyridin-1-yl)-2-methylazetidin-3-ol |
| | (Preparation 213); Amine-5; BrettP Pd G3, 100° C. |
| | HPLC-23, Gradient (% organic): 28-40% |
| | Yellow solid (38 mg, 24%). LCMS m/z = 464 [M + H]⁺; ¹HNMR (300 MHz, |
| | DMSO-d₆) δ: 10.06 (s, 1H), 9.05 (s, 1H), 8.59 (s, 1H), 8.13-7.95 (m, 2H), |
| | 6.43 (d, 1H), 5.61 (d, 1H), 4.86 (t, 1H), 4.54-4.33 (m, 2H), 4.33-4.04 (m, 3H), |
| | 3.77 (dd, 1H), 3.47 (t, 2H), 3.32-3.25 (m, 1H), 1.63-1.38 (m, 7H), 1.31 (dd, |
| | 6H), 1.19 (s, 3H). |
| 129 | 1-(4-((5-isopropyl-8-((2R,3S)-2-methyl-3-(1H-1,2,3-triazol-1-yl)azetidin-1- |
| | yl)isoquinolin-3-yl)amino)pyrimidin-2-yl)-4-methylpiperidin-4-ol |
| | 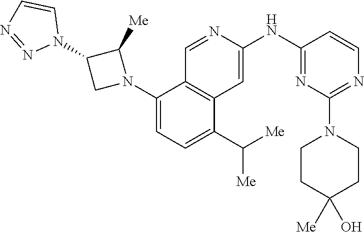 [SMILES: CC(C)c1ccc(N2C[C@H](n3ccnn3)[C@H]2C)c2cnc(Nc3ccnc(N4CCC(C)(O)CC4)n3)cc12] |
| | RCl: 3-chloro-5-isopropyl-8-((2R,3S)-2-methyl-3-(1H-1,2,3-triazol-1-yl)azetidin-1- |
| | yl)isoquinoline (Preparation 128); Amine-5; BrettP Pd G4, 90° C. |
| | ISCO 0-10% MeOH/DCM |
| | Yellow solid (9.2 mg, 26%). LCMS m/z = 514 [M + H]⁺; ¹HNMR (400 MHz, |
| | DMSO-d₆) δ: 9.86 (s, 1H), 9.11 (s, 1H), 8.73 (s, 1H), 8.40 (d, 1H), 7.97 (d, |
| | 1H), 7.79 (d, 1H), 7.44 (d, 1H), 6.71 (d, 1H), 6.38 (d, 1H), 5.35 (q, 1H), 4.97 |
| | (t, 1H), 4.74-4.50 (m, 1H), 4.30-4.11 (m, 4H), 3.58-3.38 (m, 3H), 1.65-1.41 |
| | (m, 8H), 1.29 (dd, 7H), 1.17 (s, 4H). |
| 130 | 1-(4-((5-isopropyl-8-((2R,3S)-2-methyl-3-(1H-1,2,3-triazol-1-yl)azetidin-1-yl)-2,7- |
| | naphthyridin-3-yl)amino)pyrimidin-2-yl)-4-methylpiperidin-4-ol |
| | 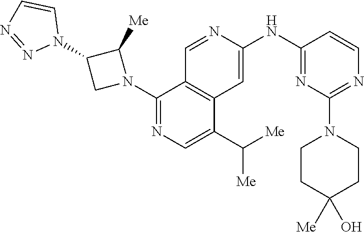 [SMILES: CC(C)c1cnc(N2C[C@H](n3ccnn3)[C@H]2C)c2cnc(Nc3ccnc(N4CCC(C)(O)CC4)n3)cc12] |
| | RCl: 6-chloro-4-isopropyl-1-((2R,3S)-2-methyl-3-(1H-1,2,3-triazol-1-yl)azetidin-1- |
| | yl)-2,7-naphthyridine (Preparation 200); Amine-5; BrettP Pd G4, 90° C. |
| | ISCO 0-10% MeOH/DCM |
| | Yellow solid (14.8 mg, 33%). LCMS m/z = 515 [M + H]⁺; ¹HNMR (400 MHz, |
| | DMSO-d₆) δ: 10.07 (s, 1H), 9.10 (s, 1H), 8.61 (s, 1H), 8.39 (d, 1H), 8.06-7.98 |
| | (m, 2H), 7.78 (d, 1H), 6.42 (d, 1H), 5.37 (q, 1H), 5.19 (t, 1H), 4.97 (t, 1H), |
| | 4.54-4.46 (m, 1H), 4.39 (s, 1H), 4.23 (d, 2H), 3.48 (d, 2H), 1.56 (d, 3H), 1.50 |
| | (d, 4H), 1.31 (dd, 6H). (signal for muliplet 2H under solvent peak). |
| 131 | (R)-4-(hydroxymethyl)-1-(4-((5-isopropyl-8-(2-methylazetidin-1-yl)-2,7- |
| | naphthyridin-3-yl)amino)pyrimidin-2-yl)piperidin-4-ol |
| | 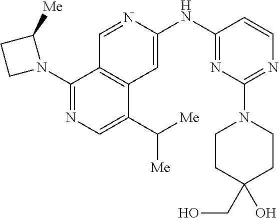 [SMILES: CC(C)c1cnc(N2CC[C@H]2C)c2cnc(Nc3ccnc(N4CCC(O)(CO)CC4)n3)cc12] |
| | RCl: (R)-6-chloro-4-isopropyl-1-(2-methylazetidin-1-yl)-2,7-naphthyridine |
| | (Preparation 191); Amine-6; XPhos Pd G4, 90° C. |
| | ISCO (0-10% MeOH/MeCN) |
| | Yellow solid (3.2 mg, 6%). LCMS m/z = 464 [M + H]⁺; ¹HNMR (400 MHz, DMSO- |
| | d₆) δ: 9.99 (s, 1H), 9.00 (s, 1H), 8.55 (s, 1H), 8.00 (d, 1H), 7.96 (s, 1H), 6.41 |
| | (d, 1H), 4.81-4.72 (m, 1H), 4.64-4.53 (m, 2H), 4.45 (d, 2H), 4.29 (s, 1H), 4.08 |
| | (q, 1H), 3.21 (d, 2H), 2.10-2.00 (m, 1H), 1.66-1.52 (m, 2H), 1.41 (d, 5H), |
| | 1.28 (dd, 6H). |
| 132 | 6-(6-((2-((4S,5R)-5-fluoro-4-hydroxy-3,3-dimethylpiperidin-1-yl)pyrimidin-4- |
| | yl)amino)-4-isopropyl-2,7-naphthyridin-1-yl)-1-thia-6-azaspiro[3.3]heptane |
| | 1,1-dioxide or 6-(6-((2-((4R,5S)-5-fluoro-4-hydroxy-3,3-dimethylpiperidin-1- |
| | yl)pyrimidin-4-yl)amino)-4-isopropyl-2,7-naphthyridin-1-yl)-1-thia-6- |
| | azaspiro[3.3]heptane 1,1-dioxide |
| | 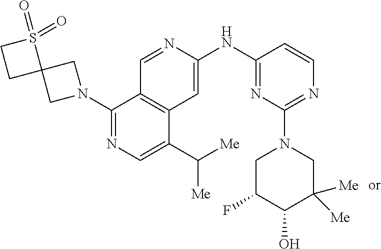 [SMILES: CC(C)c1cnc(N2CC3(CCS3(=O)=O)C2)c2cnc(Nc3ccnc(N4C[C@@H](F)[C@@H](O)C(C)(C)C4)n3)cc12] |
| | 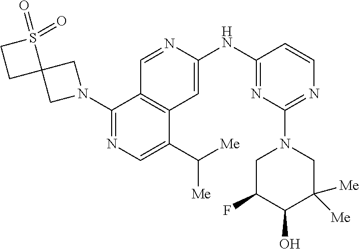 [SMILES: CC(C)c1cnc(N2CC3(CCS3(=O)=O)C2)c2cnc(Nc3ccnc(N4C[C@H](F)[C@H](O)C(C)(C)C4)n3)cc12] |
| | RCl: 6-(6-chloro-4-isopropyl-2,7-naphthyridin-1-yl)-1-thia-6-azaspiro[3.3]heptane |
| | 1,1-dioxide (Preparation 217); Amine-7; BrettPhos Pd G3, 110° C. |
| | HPLC-12, Gradient (% organic): 30-57%. |
| | Yellow solid (10 mg, 31%). LCMS m/z = 556 [M + H]⁺; ¹HNMR (400 MHz, |
| | DMSO-d₆) δ: 10.17 (s, 1H), 9.05 (s, 1H), 8.52 (s, 1H), 8.07-8.01 (m, 2H), |
| | 6.50 (d, 1H), 5.14 (d, 1H), 4.85-4.74 (m, 3H), 4.73-4.67 (m, 1H), 4.63 (d, |
| | 2H), 4.23 (s, 1H), 4.19-4.10 (m, 2H), 3.98 (dd, 1H), 3.81 (d, 1H), 3.55-3.32 |
| | (m, 3H), 2.50-2.41 (m, 2H), 1.32 (t, 6H), 0.96-0.90 (m, 6H). |
| 133 | (3S,4R,5R)-3,5-difluoro-1-(4-((8-((2R,3S)-3-hydroxy-2-methylazetidin-1-yl)-5- |
| | isopropyl-2,7-naphthyridin-3-yl)amino)pyrimidin-2-yl)-3-methylpiperidin-4- |
| | ol or (3R,4S,5S)-3,5-difluoro-1-(4-((8-((2R,3S)-3-hydroxy-2-methylazetidin- |
| | 1-yl)-5-isopropyl-2,7-naphthyridin-3-yl)amino)pyrimidin-2-yl)-3- |
| | methylpiperidin-4-ol |
| | 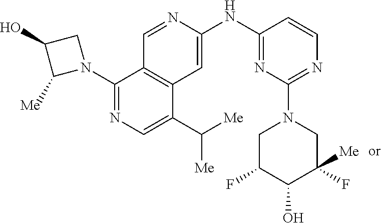 [SMILES: CC(C)c1cnc(N2C[C@H](O)[C@H]2C)c2cnc(Nc3ccnc(N4C[C@@H](F)[C@@H](O)[C@@](C)(F)C4)n3)cc12] |
| | 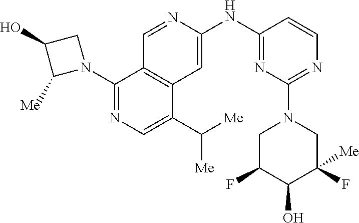 [SMILES: CC(C)c1cnc(N2C[C@H](O)[C@H]2C)c2cnc(Nc3ccnc(N4C[C@H](F)[C@H](O)[C@](C)(F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-8 ; BrettPhos Pd G3, 110° C. |
| | HPLC-23, Gradient (% organic): 28-45%. |
| | Yellow solid. LCMS m/z = 500 [M + H]⁺; ¹HNMR (300 MHz, DMSO-d₆) δ: 10.16 |
| | (s, 1H), 9.07 (s, 1H), 8.44 (s, 1H), 8.11-7.99 (m, 2H), 6.61 (d, 1H), 5.70 (d, |
| | 1H), 5.60 (d, 1H), 5.01 (s, 1H), 4.90-4.77 (m, 2H), 4.63 (s, 1H), 4.46-4.36 (m, |
| | 1H), 4.22-4.13 (m, 1H), 3.78 (dd, 2H), 3.65 (s, 1H), 3.18 (d, 2H), 1.47-1.28 |
| | (m, 12H). |
| 134 | (3R,4S,5S)-3,5-difluoro-1-(4-((8-((2R,3S)-3-hydroxy-2-methylazetidin-1-yl)-5- |
| | isopropyl-2,7-naphthyridin-3-yl)amino)pyrimidin-2-yl)-3-methylpiperidin-4- |
| | ol or (3S,4R,5R)-3,5-difluoro-1-(4-((8-((2R,3S)-3-hydroxy-2-methylazetidin- |
| | 1-yl)-5-isopropyl-2,7-naphthyridin-3-yl)amino)pyrimidin-2-yl)-3- |
| | methylpiperidin-4-ol |
| | 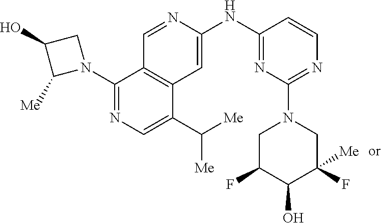 [SMILES: CC(C)c1cnc(N2C[C@H](O)[C@H]2C)c2cnc(Nc3ccnc(N4C[C@H](F)[C@H](O)[C@](C)(F)C4)n3)cc12] |
| | 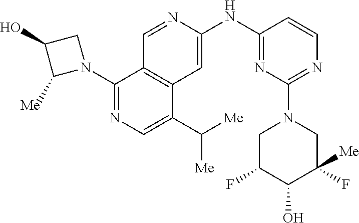 [SMILES: CC(C)c1cnc(N2C[C@H](O)[C@H]2C)c2cnc(Nc3ccnc(N4C[C@@H](F)[C@@H](O)[C@@](C)(F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-9 ; BrettPhos Pd G3, 110° C. |
| | HPLC-23, Gradient (% organic): 28-45%. |
| | Yellow solid. LCMS m/z = 500 [M + H]⁺; ¹HNMR (300 MHz, DMSO-d₆) δ: 10.15 |
| | (s, 1H), 9.07 (s, 1H), 8.44 (s, 1H), 8.11-7.99 (m, 2H), 6.61 (d, 1H), 5.70 (d, |
| | 1H), 5.60 (d, 1H), 5.02 (d, 1H), 4.92-4.76 (m, 2H), 4.62 (s, 1H), 4.46-4.36 (m, |
| | 1H), 4.22-4.12 (m, 1H), 3.78 (dd, 1H), 3.72-3.57 (m, 1H), 3.38-3.23 (m, 1H), |
| | 3.15 (t, 2H), 1.48-1.28 (m, 12H). |
| 135 | (3S,4S,5S)-3,5-difluoro-1-(4-((8-((2R,3S)-3-hydroxy-2-methylazetidin-1-yl)-5- |
| | isopropyl-2,7-naphthyridin-3-yl)amino)pyrimidin-2-yl)-3-methylpiperidin-4- |
| | ol or (3R,4R,5R)-3,5-difluoro-1-(4-((8-((2R,3S)-3-hydroxy-2-methylazetidin- |
| | 1-yl)-5-isopropyl-2,7-naphthyridin-3-yl)amino)pyrimidin-2-yl)-3- |
| | methylpiperidin-4-ol |
| | 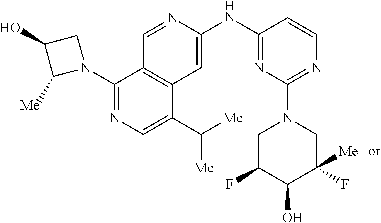 [SMILES: CC(C)c1cnc(N2C[C@H](O)[C@H]2C)c2cnc(Nc3ccnc(N4C[C@H](F)[C@H](O)[C@@](C)(F)C4)n3)cc12] |
| | 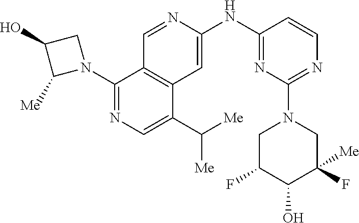 [SMILES: CC(C)c1cnc(N2C[C@H](O)[C@H]2C)c2cnc(Nc3ccnc(N4C[C@@H](F)[C@@H](O)[C@](C)(F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-10. BrettPhos Pd G3, 110° C. |
| | HPLC-23, Gradient (% organic): 28-45%. |
| | Yellow solid. LCMS m/z = 500 [M + H]⁺; ¹HNMR (300 MHz, DMSO-d₆) δ: 10.15 |
| | (s, 1H), 9.07 (s, 1H), 8.46 (s, 1H), 8.11-7.99 (m, 2H), 6.59 (d, 1H), 5.90 (d, |
| | 1H), 5.60 (d, 1H), 4.87 (t, 2H), 4.72 (s, 1H), 4.40 (q, 1H), 4.30-4.06 (m, 2H), |
| | 4.00-3.88 (m, 2H), 3.78 (dd, 2H), 3.42-3.31 (m, 1H), 1.46-1.38 (m, 3H), 1.38- |
| | 1.27 (m, 9H). |
| 136 | (3R,4R,5R)-3,5-difluoro-1-(4-((8-((2R,3S)-3-hydroxy-2-methylazetidin-1-yl)-5- |
| | isopropyl-2,7-naphthyridin-3-yl)amino)pyrimidin-2-yl)-3-methylpiperidin-4- |
| | ol or (3S,4S,5S)-3,5-difluoro-1-(4-((8-((2R,3S)-3-hydroxy-2-methylazetidin- |
| | 1-yl)-5-isopropyl-2,7-naphthyridin-3-yl)amino)pyrimidin-2-yl)-3- |
| | methylpiperidin-4-ol |
| | 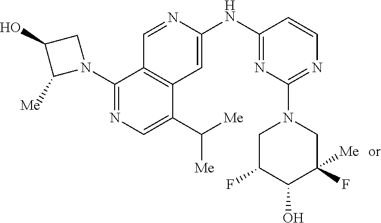 [SMILES: CC(C)c1cnc(N2C[C@H](O)[C@H]2C)c2cnc(Nc3ccnc(N4C[C@@H](F)[C@@H](O)[C@](C)(F)C4)n3)cc12] |
| | 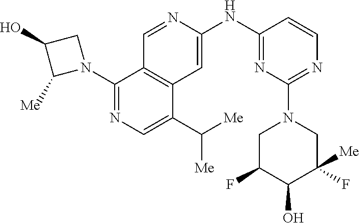 [SMILES: CC(C)c1cnc(N2C[C@H](O)[C@H]2C)c2cnc(Nc3ccnc(N4C[C@H](F)[C@H](O)[C@@](C)(F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-11; BrettPhos Pd G3, 110° C. |
| | HPLC-23, Gradient (% organic): 28-45%. |
| | Yellow solid. LCMS m/z = 500 [M + H]⁺; ¹HNMR (300 MHz, DMSO-d₆) δ: 10.15 |
| | (s, 1H), 9.07 (s, 1H), 8.46 (s, 1H), 8.11-7.99 (m, 2H), 6.59 (d, 1H), 5.89 (d, |
| | 1H), 5.60 (d, 1H), 4.87 (t, 2H), 4.45-4.36 (m, 2H), 4.22-4.04 (m, 1H), 3.98 (d, |
| | 1H), 3.91 (s, 2H), 3.78 (dd, 2H), 1.46-1.38 (m, 3H), 1.38-1.27 (m,9H). |
| 137 | 2-(((2R,3S)-1-(6-((2-((3R,4S)-3-fluoro-4-(methoxy-d3)piperidin-1-yl)pyrimidin-4- |
| | yl)amino)-4-isopropyl-2,7-naphthyridin-1-yl)-2-methylazetidin-3- |
| | yl)oxy)ethan-1-ol |
| | 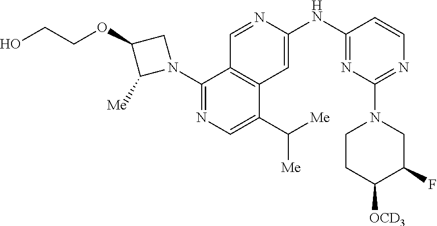 |
| | RCl: 2-(((2R,3S)-1-(6-chloro-4-isopropyl-2,7-naphthyridin-1-yl)-2-methylazetidin- |
| | 3-yl)oxy)ethan-1-ol (Preparation 234); Amine-12. BrettPhos Pd G3, 110° C. |
| | Prep-TLC (20:1 DCM/MeOH) followed by HPLC-18, Gradient (% organic) 36- |
| | 46%. |
| | White solid (55 mg, 43%). LCMS m/z = 529 [M + H]⁺; ¹HNMR (300 MHz, DMSO- |
| | d₆) δ: 10.12 (s, 1H), 9.06 (s, 1H), 8.52 (s, 1H), 8.14-7.96 (m, 2H), 6.53 (d, |
| | 1H), 5.12-4.79 (m, 2H), 4.80-4.60 (m, 2H), 4.56-4.35 (m, 2H), 4.11 (q, 1H), |
| | 3.87 (dd, 1H), 3.77-3.37 (m, 6H), 3.30 (s, 2H), 1.86-1.69 (m, 2H), 1.46 (d, |
| | 3H), 1.33 (dd, 6H). |
| 138 | (2R,3S)-1-(6-((2-((3R,4S)-3-fluoro-4-(methoxy-d3)piperidin-1-yl)pyrimidin-4- |
| | yl)amino)-4-isopropyl-2,7-naphthyridin-1-yl)-2-methylazetidin-3-ol |
| | 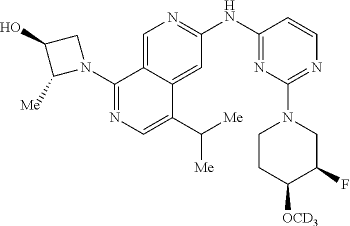 |
| | RCl: (2R,3S)-1-(6-chloro-4-isopropyl-2,7-naphthyridin-1-yl)-2-methylazetidin-3-ol |
| | (Preparation 213); Amine-12; XPhos Pd G3, 100° C. |
| | Prep-TLC (20:1 DCM/MeOH) followed by HPLC-18, Gradient (% organic): 40- |
| | 42%. |
| | White solid (40 mg, 30%). LCMS m/z = 485 [M + H]⁺; ¹HNMR (300 MHz, DMSO- |
| | d₆) δ: 10.09 (s, 1H), 9.06 (s, 1H), 8.51 (s, 1H), 8.09-7.99 (m, 2H), 6.53 (d, |
| | 1H), 5.59 (s, 1H), 5.14-4.82 (m, 2H), 4.80-4.58 (m, 1H), 4.58-4.28 (m, 2H), |
| | 4.17 (s, 1H), 3.78 (dd, 1H), 3.49 (dd, 2H), 3.28 (d, 2H), 1.89-1.69 (m, 2H), |
| | 1.42-1.32 (m, 9H). |
| 139 | (2R,3S)-1-(6-((2-((3S,4R)-3-fluoro-4-(methoxy-d3)piperidin-1-yl)pyrimidin-4- |
| | yl)amino)-4-isopropyl-2,7-naphthyridin-1-yl)-2-methylazetidin-3-ol |
| | 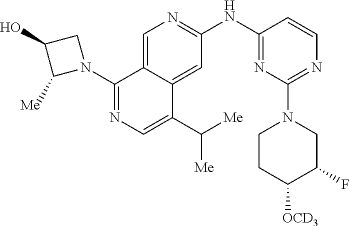 |
| | RCl: (2R,3S)-1-(6-chloro-4-isopropyl-2,7-naphthyridin-1-yl)-2-methylazetidin-3-ol |
| | (Preparation 213); Amine-13; XPhos Pd G3, 100° C. |
| | Prep-TLC (20:1 DCM/MeOH) followed by HPLC-18, Gradient (% organic): 37- |
| | 45%. |
| | White solid (30 mg, 22%). LCMS m/z = 485 [M + H]⁺; ¹HNMR (300 MHz, DMSO- |
| | d₆) δ: 10.09 (s, 1H), 9.06 (s, 1H), 8.51 (s, 1H), 8.14-7.91 (m, 2H), 6.53 (d, |
| | 1H), 5.59 (d, 1H), 5.08-4.79 (m, 2H), 4.70 (d, 1H), 4.53-4.33 (m, 2H), 4.17 |
| | (p, 1H), 3.78 (dd, 1H), 3.54 (td, 2H), 3.31 (m, 2H), 1.78 (dd, 2H), 1.51-1.19 |
| | (m, 9H). |
| 140 | 2-(((2R,3S)-1-(6-((2-((3S,4R)-3-fluoro-4-(methoxy-d3)piperidin-1-yl)pyrimidin-4- |
| | yl)amino)-4-isopropyl-2,7-naphthyridin-1-yl)-2-methylazetidin-3- |
| | yl)oxy)ethan-1-ol |
| | 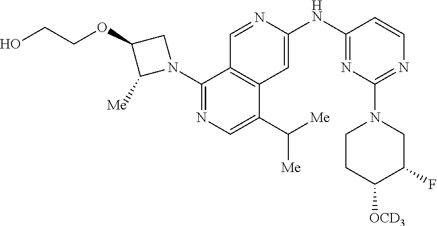 |
| | RCl: 2-(((2R,3S)-1-(6-chloro-4-isopropyl-2,7-naphthyridin-1-yl)-2-methylazetidin- |
| | 3-yl)oxy)ethan-1-ol (Preparation 234); Amine-13; BrettPhos Pd G3, 110° C. |
| | Prep-TLC (20:1 DCM/MeOH) followed by HPLC-18, Gradient (% organic): 37- |
| | 46%. |
| | White solid (50 mg, 40%). LCMS m/z = 529 [M + H]⁺; ¹HNMR (300 MHz, DMSO- |
| | d₆) δ: 10.11 (s, 1H), 9.06 (s, 1H), 8.52 (s, 1H), 8.12-7.96 (m, 2H), 6.53 (d, |
| | 1H), 5.08-4.85 (m, 2H), 4.79-4.61 (m, 2H), 4.58-4.37 (m, 2H), 4.10 (q, 1H), |
| | 3.87 (dd, 1H), 3.66-3.42 (m, 6H), 3.30 (s, 2H), 1.89-1.68 (m, 2H), 1.45 (d, |
| | 3H), 1.33 (dd, 6H). |
| 141 | 2-(((3R,4S)-3-fluoro-1-(4-((5-isopropyl-8-((R)-2-methylazetidin-1-yl)-2,7- |
| | naphthyridin-3-yl)amino)pyrimidin-2-yl)piperidin-4-yl)oxy)ethan-1-ol |
| | 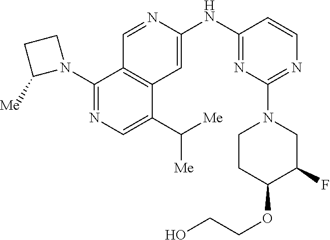 [SMILES: CC(C)c1cnc(N2CC[C@H]2C)c2cnc(Nc3ccnc(N4CC[C@H](OCCO)[C@H](F)C4)n3)cc12] |
| | RCl: (R)-6-chloro-4-isopropyl-1-(2-methylazetidin-1-yl)-2,7-naphthyridine |
| | (Preparation 191); Amine-14; BrettPhos Pd G3, 110° C. |
| | Prep-TLC (15:1 EtOAc/MeOH) followed by HPLC-18, Gradient (% organic): 38- |
| | 48%. |
| | Yellow solid. LCMS m/z = 496 [M + H]⁺; ¹HNMR (400 MHz, DMSO-d₆) δ: 10.07 |
| | (s, 1H), 9.03 (s, 1H), 8.49 (s, 1H), 8.04 (d, 1H), 7.99 (s, 1H), 6.52 (d, 1H), |
| | 4.99 (d, 1H), 4.87 (s, 1H), 4.79 (t, 1H), 4.75 (s, 1H), 4.64 (q, 1H), 4.51 (d, |
| | 1H), 4.10 (q, 1H), 3.80-3.73 (m, 1H), 3.58 (q, 4H), 3.57-3.45 (m, 1H), 3.27 |
| | (d, 3H), 2.06 (h, 1H), 1.87-1.79 (m, 2H), 1.75 (d, 3H,), 1.43 (d, 6H). |
| 142 | 2-(((3S,4R)-3-fluoro-1-(4-((5-isopropyl-8-((R)-2-methylazetidin-1-yl)-2,7- |
| | naphthyridin-3-yl)amino)pyrimidin-2-yl)piperidin-4-yl)oxy)ethan-1-ol |
| | 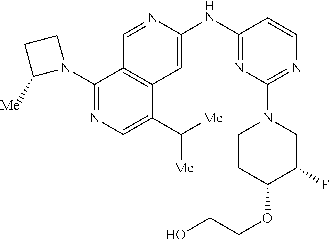 [SMILES: CC(C)c1cnc(N2CC[C@H]2C)c2cnc(Nc3ccnc(N4CC[C@@H](OCCO)[C@@H](F)C4)n3)cc12] |
| | RCl: (R)-6-chloro-4-isopropyl-1-(2-methylazetidin-1-yl)-2,7-naphthyridine |
| | (Preparation 191); Amine-15; BrettPhos Pd G3, 100° C. |
| | Prep-TLC (15:1 DCM/MeOH) |
| | Yellow solid (150 mg, 33%). LCMS m/z = 496 [M + H]⁺; ¹HNMR (300 MHz, |
| | DMSO-d₆) δ: 10.08 (s, 1H), 9.03 (s, 1H), 8.49 (s, 1H), 8.04 (d, 1H), 7.98 (s, |
| | 1H), 6.51 (d, 1H), 4.99 (s, 1H), 4.78 (h, 2H), 4.68-4.58 (m, 2H), 4.51 (d, 1H), |
| | 4.10 (q, 1H), 3.73 (dd, 1H), 3.64-3.49 (m, 4H), 3.54 (s, 1H), 3.24 (d), 2.13- |
| | 2.00 (m, 1H), 1.88-1.79 (m, 1H), 1.75 (d, 1H), 1.43 (d, 3H), 1.32 (dd, 6H). |
| 143 | (3R,4S)-3-fluoro-1-(4-((8-((2R,3S)-3-hydroxy-2-methylazetidin-1-yl)-5-isopropyl- |
| | 2,7-naphthyridin-3-yl)amino)pyrimidin-2-yl)piperidin-4-ol |
| | 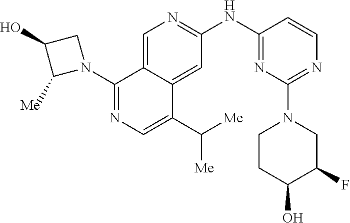 [SMILES: CC(C)c1cnc(N2C[C@H](O)[C@H]2C)c2cnc(Nc3ccnc(N4CC[C@H](O)[C@H](F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-16; BrettPhos Pd G3, 110° C. |
| | Prep-TLC (20:1 DCM/MeOH) followed by HPLC-18, Gradient (% organic) 26- |
| | 36%. |
| | Yellow solid. LCMS m/z = 468 [M + H]⁺; ¹HNMR (300 MHz, DMSO-d₆) δ: 10.08 |
| | (s, 1H), 9.06 (s, 1H), 8.54 (s, 1H), 8.09-7.99 (m, 2H), 6.50 (d, 1H), 5.60 |
| | (d,1H), 5.14 (d, 1H), 4.91-4.50 (m, 3H), 4.40 (q, 2H), 4.18 (h, 1H), 3.96-3.72 |
| | (m, 2H), 3.57 (dd, 1H), 3.33 (m, 2H), 1.73 (s, 2H), 1.42-1.33 (m, 9H). |
| 144 | (3S,4R)-3-fluoro-1-(4-((5-isopropyl-8-((2R,3S)-2-methyl-3-(1H-1,2,3-triazol-1- |
| | yl)azetidin-1-yl)-2,7-naphthyridin-3-yl)amino)pyrimidin-2-yl)-4- |
| | methylpiperidin-4-ol |
| | 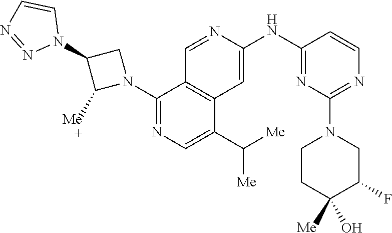 [SMILES: CC(C)c1cnc(N2C[C@H](n3ccnn3)[C@H]2C)c2cnc(Nc3ccnc(N4CC[C@@](C)(O)[C@@H](F)C4)n3)cc12] |
| | RCl: 6-chloro-4-isopropyl-1-((2R,3S)-2-methyl-3-(1H-1,2,3-triazol-1-yl)azetidin-1- |
| | yl)-2,7-naphthyridine (Preparation 200); Amine-17; XPhos Pd G4, 90° C. |
| | ISCO (SiO₂, 0-100% EtOAc/Hex then 0-20% MeOH/DCM) |
| | White solid (7.3 mg, 18.3%). LCMS m/z = 533 [M + H]⁺; ¹HNMR (400 MHz, |
| | DMSO-d₆) δ: 10.14 (s, 1H), 9.11 (s, 1H), 8.56 (s, 1H), 8.39 (d, 1H), 8.07-8.01 |
| | (m, 2H), 7.77 (d, 1H), 6.50 (d, 1H), 5.37 (q, 1H), 5.20 (t, 1H), 4.97 (t, 1H), |
| | 4.83 (s, 1H), 4.54-4.46 (m, 1H), 4.40 (d, 1H), 4.16 (d, 1H), 3.60 (s, 1H), 3.49 |
| | (s, 1H), 3.38-3.30 (m, 1H), 1.69 (s, 1H), 1.56 (d, 3H), 1.32 (t, 6H), 1.23 (d, |
| | 6H). |
| 145 | (4S,5R)-5-fluoro-1-(4-((8-((2R,3S)-3-hydroxy-2-methylazetidin-1-yl)-5-isopropyl- |
| | 2,7-naphthyridin-3-yl)amino)pyrimidin-2-yl)-3,3-dimethylpiperidin-4-ol or |
| | (4R,5S)-5-fluoro-1-(4-((8-((2R,3S)-3-hydroxy-2-methylazetidin-1-yl)-5- |
| | isopropyl-2,7-naphthyridin-3-yl)amino)pyrimidin-2-yl)-3,3- |
| | dimethylpiperidin-4-ol |
| | 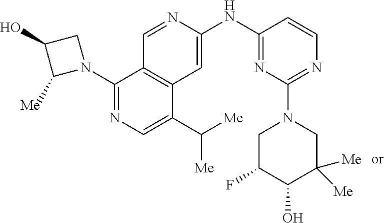 [SMILES: CC(C)c1cnc(N2C[C@H](O)[C@H]2C)c2cnc(Nc3ccnc(N4C[C@@H](F)[C@@H](O)C(C)(C)C4)n3)cc12] |
| | 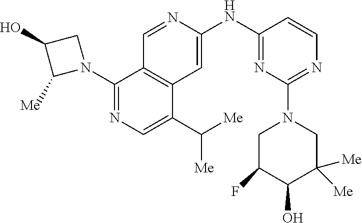 [SMILES: CC(C)c1cnc(N2C[C@H](O)[C@H]2C)c2cnc(Nc3ccnc(N4C[C@H](F)[C@H](O)C(C)(C)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-7; RuPhos Pd G3, 90° C. |
| | LCMS m/z = 496 [M + H]⁺; ¹HNMR (500 MHz, DMSO-d₆) δ: 10.08 (s, 1H), 9.08 (s, |
| | 1H), 8.49 (s, 1H), 8.08-8.00 (m, 2H), 6.54 (d, 1H), 5.61 (d, 1H), 5.15 (d, 1H), |
| | 4.95-4.65 (m, 2H), 4.50-4.36 (m, 1H), 4.24 (d, 1H), 4.06-3.91 (m, 1H), 3.90- |
| | 3.70 (m, 2H), 3.59 (s, 1H), 3.54-3.35 (m, 3H), 1.44 (d, 3H), 1.33 (t, 6H), 1.00- |
| | 0.90 (m, 6H). |
| 146 | (R)-1-(6-((2-(5,5-difluoro-1-oxa-7-azaspiro[3.5]nonan-7-yl)pyrimidin-4-yl)amino)- |
| | 4-isopropyl-2,7-naphthyridin-1-yl)azetidin-3-ol or (S)-1-(6-((2-(5,5-difluoro- |
| | 1-oxa-7-azaspiro[3.5]nonan-7-yl)pyrimidin-4-yl)amino)-4-isopropyl-2,7- |
| | naphthyridin-1-yl)azetidin-3-ol |
| | 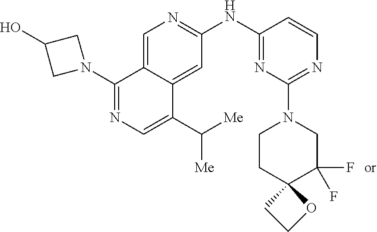 [SMILES: CC(C)c1cnc(N2CC(O)C2)c2cnc(Nc3ccnc(N4CC[C@@]5(CCO5)C(F)(F)C4)n3)cc12] |
| | 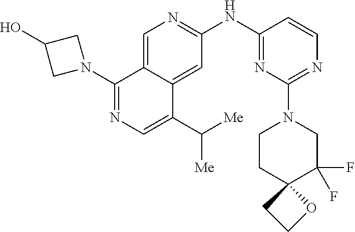 [SMILES: CC(C)c1cnc(N2CC(O)C2)c2cnc(Nc3ccnc(N4CC[C@]5(CCO5)C(F)(F)C4)n3)cc12] |
| | RCl: 1-(6-chloro-4-isopropyl-2,7-naphthyridin-1-yl)azetidin-3-ol (Preparation 161); |
| | Amine-18; BrettPhos Pd G3, 100° C. |
| | Prep-TLC (2:1 DCM/MeOH) followed by prep-HPLC-10: Gradient (% organic): |
| | 43-53%. White solid (67.3 mg, 75%). LCMS m/z = 498 [M + H]⁺; ¹HNMR |
| | (400 MHz, DMSO-d₆) δ: 10.16 (s, 1H), 9.05 (s, 1H), 8.41 (s, 1H), 8.08 (d, |
| | 1H), 7.98 (s, 1H), 6.62 (d, 1H), 5.70 (d, 1H), 4.69-4.55 (m, 3H), 4.51 (t, 2H), |
| | 4.40 (d, 1H), 4.10-395 (m, 3H), 3.67 (t, 1H), 3.37-3.32 (m, 1H), 2.79 (dt, 1H), |
| | 2.51 (d, 2H), 2.11 (s, 1H), 2.06-1.95 (m, 1H), 1.33 (d, 6H). |
| 147 | 1-(6-((2-((3S,4R)-3-fluoro-4-methoxypiperidin-1-yl)pyrimidin-4-yl)amino)-4- |
| | isopropyl-2,7-naphthyridin-1-yl)azetidin-3-ol |
| | 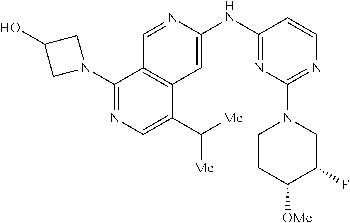 [SMILES: CO[C@@H]1CCN(c2nccc(Nc3cc4c(C(C)C)cnc(N5CC(O)C5)c4cn3)n2)C[C@@H]1F] |
| | RCl: 1-(6-chloro-4-isopropyl-2,7-naphthyridin-1-yl)azetidin-3-ol (Preparation 161); |
| | Amine-19 |
| | HPLC-18, Gradient (% organic): 27-43% |
| | White solid (35.1 mg, 14%). LCMS m/z = 468 [M + H]⁺ ¹HNMR (400 MHz, |
| | DMSO-d₆) δ: 10.08 (s, 1H), 9.03 (d, 1H), 8.48 (s, 1H), 8.05 (d, 1H), 7.97 (s, |
| | 1H), 6.53 (d, 1H), 5.69 (d, 1H), 5.10-4.83 (m, 1H), 4.72 (ddd, 1H), 4.63-4.53 |
| | (m, 3H), 4.47 (d, 1H), 4.09 (dd, 2H), 3.69-3.39 (m, 2H), 3.37 (s, 3H), 3.29- |
| | 3.21 (m, 1H), 1.84-1.69 (m, 2H), 1.32 (dd, 6H). |
| 148 | 2-(((2R,3S)-1-(6-((2-((3S,4R)-3-fluoro-4-methoxypiperidin-1-yl)pyrimidin-4- |
| | yl)amino)-4-isopropyl-2,7-naphthyridin-1-yl)-2-methylazetidin-3- |
| | yl)oxy)ethan-1-ol |
| | 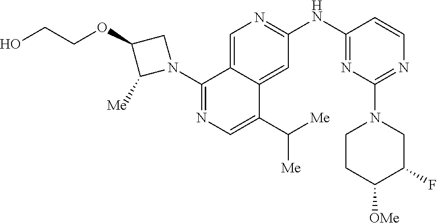 [SMILES: CO[C@@H]1CCN(c2nccc(Nc3cc4c(C(C)C)cnc(N5C[C@H](OCCO)[C@H]5C)c4cn3)n2)C[C@@H]1F] |
| | RCl: 2-(((2R,3S)-1-(6-chloro-4-isopropyl-2,7-naphthyridin-1-yl)-2-methylazetidin- |
| | 3-yl)oxy)ethan-1-ol (Preparation 234); Amine-19; XantPhos Pd G2 |
| | HPLC-18, Gradient (% organic): 39-46% |
| | White solid. LCMS m/z = 526 [M + H]⁺ ¹HNMR (300 MHz, DMSO-d₆) δ: 10.11 (s, |
| | 1H), 9.06 (s, 1H), 8.51 (s, 1H), 8.05 (d, 1H), 8.01 (s, 1H), 6.53 (d, 1H), 5.12- |
| | 4.77 (m, 2H), 4.78-4.59 (m, 2H), 4.57-4.32 (m, 2H), 4.11 (d, 1H), 3.87 (dd, |
| | 1H), 3.71-3.33 (m, 11H), 2.00-1.63 (m, 2H), 1.46 (d, 3H,), 1.33 (dd, 6H). |
| 149 | (2R,3S)-1-(6-((2-((3R,4S)-3-fluoro-4-methoxypiperidin-1-yl)pyrimidin-4- |
| | yl)amino)-4-isopropyl-2,7-naphthyridin-1-yl)-2-methylazetidin-3-ol |
| | 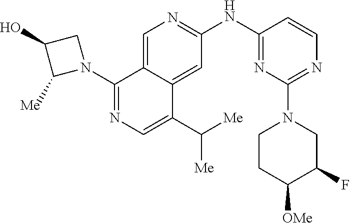 [SMILES: CO[C@H]1CCN(c2nccc(Nc3cc4c(C(C)C)cnc(N5C[C@H](O)[C@H]5C)c4cn3)n2)C[C@H]1F] |
| | RCl: (2R,3S)-1-(6-chloro-4-isopropyl-2,7-naphthyridin-1-yl)-2-methylazetidin-3-ol |
| | (Preparation 213); 2-((3R,4S)-3-fluoro-4-methoxypiperidin-1-yl)pyrimidin-4- |
| | amine (Preparation 32); XantPhos Pd G2 |
| | HPLC-18, Gradient (% organic): 38-46% |
| | White solid (30 mg, 23%). LCMS m/z = 482 [M + H]⁺ ¹HNMR (300 MHz, DMSO- |
| | d₆) δ: 10.09 (s, 1H), 9.06 (s, 1H), 8.51 (s, 1H), 8.09-7.99 (m, 2H), 6.53 (d, |
| | 1H), 5.59 (s, 1H), 5.14-4.82 (m, 2H), 4.80-4.58 (m, 1H), 4.58-4.28 (m, 2H), |
| | 4.17 (s, 1H), 3.78 (dd, 3H), 3.49 (dd, 3H), 3.28 (d, 2H), 1.89-1.69 (m, 2H), |
| | 1.42-1.32 (m, 9H). |
| 150 | (2R,3S)-1-(6-((2-((3S,4R)-3-fluoro-4-methoxypiperidin-1-yl)pyrimidin-4- |
| | yl)amino)-4-isopropyl-2,7-naphthyridin-1-yl)-2-methylazetidin-3-ol |
| | 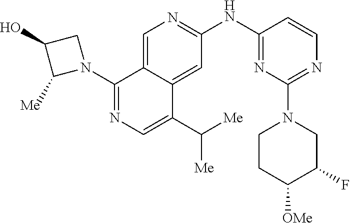 [SMILES: CO[C@@H]1CCN(c2nccc(Nc3cc4c(C(C)C)cnc(N5C[C@H](O)[C@H]5C)c4cn3)n2)C[C@@H]1F] |
| | RCl: (2R,3S)-1-(6-chloro-4-isopropyl-2,7-naphthyridin-1-yl)-2-methylazetidin-3-ol |
| | (Preparation 213); Amine-19; XantPhos Pd G2 |
| | HPLC-21, Gradient (% organic): 33-45% |
| | White solid (30 mg, 23%). LCMS m/z = 482 [M + H]⁺ ¹HNMR (300 MHz, DMSO- |
| | d₆) δ: 10.09 (s, 1H), 9.06 (s, 1H), 8.51 (s, 1H), 8.24-8.00 (m, 2H), 6.53 (d, |
| | 1H), 5.59 (d, 1H), 4.95 (d, 2H), 4.72 (m, 1H), 4.43 (t, 2H), 4.18 (d, 1H), 3.90- |
| | 3.72 (m, 1H), 3.72-3.45 (m, 2H), 3.37 (s, 3H), 3.29-3.22 (m, 2H), 1.78 (d, |
| | 2H), 1.45-1.29 (m, 9H). |
| 151 | (3S,4R)-3-fluoro-1-(4-((5-isopropyl-8-(pyrrolidin-1-yl)-2,7-naphthyridin-3- |
| | yl)amino)pyrimidin-2-yl)piperidin-4-ol |
| | 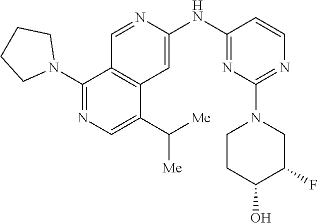 [SMILES: CC(C)c1cnc(N2CCCC2)c2cnc(Nc3ccnc(N4CC[C@@H](O)[C@@H](F)C4)n3)cc12] |
| | RCl: 6-chloro-4-isopropyl-1-(pyrrolidin-1-yl)-2,7-naphthyridine (Preparation 157); |
| | Amine-20; BrettPhos Pd G3 |
| | HPLC-12, Gradient (% organic): 38-57% |
| | White solid (39.2 mg, 27%). LCMS m/z = 452 [M + H]⁺ ¹HNMR (300 MHz, |
| | DMSO-d₆) δ: 10.03 (s, 1H), 9.28 (s, 1H), 8.45 (s, 1H), 8.02 (d, 1H), 7.94 (s, |
| | 1H), 6.48 (d, 1H), 5.12 (d, 1H), 4.83-4.46 (m, 2H), 4.35 (d, 1H), 3.98-3.66 |
| | (m, 5H), 3.59-3.33 (m, 3H), 1.92 (t, 4H), 1.77-1.63 (m, 2H), 1.30 (dd, 6H). |
| 152 | (3S,4R)-1-(4-((8-(azetidin-1-yl)-5-isopropyl-2,7-naphthyridin-3- |
| | yl)amino)pyrimidin-2-yl)-3-fluoropiperidin-4-ol |
| | 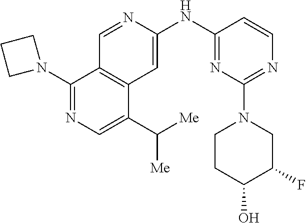 [SMILES: CC(C)c1cnc(N2CCC2)c2cnc(Nc3ccnc(N4CC[C@@H](O)[C@@H](F)C4)n3)cc12] |
| | RCl: 1-(azetidin-1-yl)-6-chloro-4-isopropyl-2,7-naphthyridine (Preparation 168); |
| | Amine-20; XPhos Pd G2 |
| | HPLC-14, Gradient (% organic): 20-50% |
| | White solid (39.2 mg, 27%). LCMS m/z = 438 [M + H]⁺ ¹HNMR (300 MHz, |
| | MeOH-d₄) δ: 9.10 (s, 1H), 8.58 (s, 1H), 8.03 (d, 1H), 7.87 (s, 1H), 6.44 (d, |
| | 1H), 4.68-4.63 (m, 2H), 4.52 (t, 4H), 4.43-4.40 (m, 1H), 4.05-3.98 (m, 1H), |
| | 3.74-3.64 (m, 1H), 3.52-3.46 (m, 2H), 2.59-2.51 (m, 2H), 1.97-1.85 (m, 2H), |
| | 1.41 (dd, 6H). |
| 153 | (3S,4R)-3-fluoro-1-(4-((5-isopropoxy-8-((R)-2-methylazetidin-1-yl)-2,7- |
| | naphthyridin-3-yl)amino)pyrimidin-2-yl)piperidin-4-ol |
| | 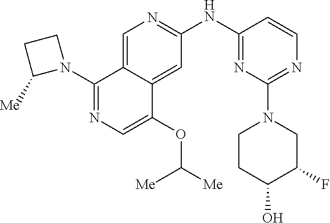 [SMILES: CC(C)Oc1cnc(N2CC[C@H]2C)c2cnc(Nc3ccnc(N4CC[C@@H](O)[C@@H](F)C4)n3)cc12] |
| | RCl: (R)-6-chloro-4-isopropoxy-1-(2-methylazetidin-1-yl)-2,7-naphthyridine |
| | (Preparation 319); Amine-20; XPhos Pd G4 |
| | ISCO (0-10% MeOH/DCM) |
| | Yellow foam (20 mg, 74%). LCMS m/z = 468 [M + H]⁺ ¹HNMR (300 MHz, |
| | DMSO-d₆) δ: 10.05 (s, 1H), 8.99 (d, 1H), 8.42 (s, 1H), 8.02 (d, 1H), 7.78 (s, |
| | 1H), 6.48 (d, 1H), 5.09 (d, 1H), 4.77-4.51 (m, 5H), 4.39-4.29 (m, 1H), 4.02- |
| | 3.93 (m, 1H), 3.82 (dt, 1H), 3.63-3.48 (m, 1H), 3.40-3.31 (m, 1H), 2.47-2.40 |
| | (m, 1H), 2.10-1.98 (m, 1H), 1.74-1.63 (m, 2H), 1.38 (d, 3H), 1.32 (d, 6H). |
| 154 | (3S,4R)-3-fluoro-1-(4-((8-((2R,3S)-3-hydroxy-2-methylazetidin-1-yl)-5-isopropyl- |
| | 2,7-naphthyridin-3-yl)amino)pyrimidin-2-yl)piperidin-4-ol |
| | 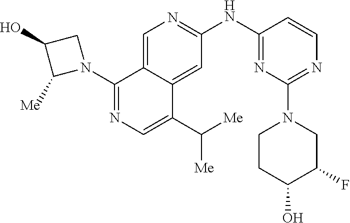 [SMILES: CC(C)c1cnc(N2C[C@H](O)[C@H]2C)c2cnc(Nc3ccnc(N4CC[C@@H](O)[C@@H](F)C4)n3)cc12] |
| | RCl: (2R,3S)-1-(6-chloro-4-isopropyl-2,7-naphthyridin-1-yl)-2-methylazetidin-3-ol |
| | (Preparation 213); Amine-20; XantPhos Pd G2 |
| | HPLC-18, Gradient (% organic): 30-38% |
| | White solid (30 mg, 22%). LCMS m/z = 468 [M + H]⁺ ¹HNMR (300 MHz, DMSO- |
| | d₆) δ: 10.08 (s, 1H), 9.06 (s, 1H), 8.54 (s, 1H), 8.09-7.99 (m, 2H), 6.50 (d, |
| | 1H), 5.60 (d, 1H), 5.14 (d, 1H), 4.91-4.50 (m, 3H), 4.40 (q, 2H), 4.18 (h, 1H), |
| | 3.96-3.72 (m, 2H), 3.57 (dd, 1H), 3.33 (m, 2H), 1.73 (s, 2H), 1.42-1.33 (m, |
| | 9H). |

### Example 155

### (2S,3R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-N,N,2-trimethylazetidine-3-carboxamide or (2R,3S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-N,N,2-trimethylazetidine-3-carboxamide

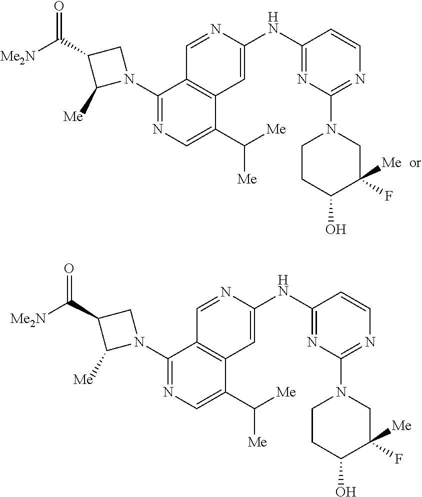 [SMILES: CC(C)c1cnc(N2C[C@@H](C(=O)N(C)C)[C@@H]2C)c2cnc(Nc3ccnc(N4CC[C@@H](O)[C@@](C)(F)C4)n3)cc12 ; CC(C)c1cnc(N2C[C@H](C(=O)N(C)C)[C@H]2C)c2cnc(Nc3ccnc(N4CC[C@@H](O)[C@@](C)(F)C4)n3)cc12]

Part 1.1-(6-((2-((3S,4R)-3-fluoro-4-hydroxy-3-methylpiperidin-1-yl)pyrimidin-4-yl)amino)-4-isopropyl-2,7-naphthyridin-1-yl)-N,N,2-trimethylazetidine-3-carboxamide was prepared from trans-rac-1-(6-chloro-4-isopropyl-2,7-naphthyridin-1-yl)-N,N,2-trimethylazetidine-3-carboxamide (Preparation 226) and (3S,4R)-1-(4-aminopyrimidin-2-yl)-3-fluoro-3-methylpiperidin-4-ol (Preparation 8) using an analogous method to that described for Example 38 (RuPhos Pd G3 method). Prep-TLC (20:1 DCM/MeOH).

Part 2: The compound of Part 1 was purified by chiral HPLC (CHIRALPAK IC; 20×250 mm, 5 mm; 30% IPA/Hex (+10 mM NH₃/MeOH) to afford the title compound as a white solid.

Peak 2: White solid (35.7 mg). LCMS m/z=537 [M+H]⁺¹HNMR (300 MHz, DMSO-d₆) δ: 1007 (s, 1H) 9.03 (s, 1H), 847 (s, 1H), 8.11-7.79 (m, 2H), 6.49 (d, 1H), 5.03 (d, 1H), 4.95-4.46 (m, 4H), 4.10 (t, 1H), 3.53 (d, 2H), 3.29 (s, 1H). 3.23-3.01 (m, 2H), 2.94 (s, 3H), 2.81 (s, 3H), 1.71 (s, 2H). 0.48 (d, 3H), 1.42-1.04 (m, 9H).

### Example 156

### (3S,4R)-3-fluoro-3-methyl-1-(4-((8-((S)-2-methylazetidin-1-yl)-5-((3-methyloxetan-3-yl)methyl)-2,7-naphthyridin-3-yl)amino)pyrimidin-2-yl)piperidin-4-ol or (3S,4R)-3-fluoro-3-methyl-1-(4-((8-((R)-2-methylazetidin-1-yl)-5-((3-methyloxetan-3-yl)methyl)-2,7-naphthyridin-3-yl)amino)pyrimidin-2-yl)piperidin-4-ol

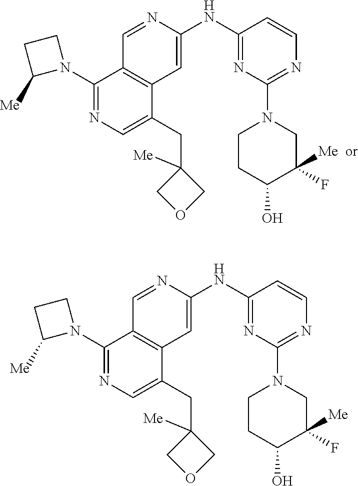 [SMILES: C[C@H]1CCN1c1ncc(CC2(C)COC2)c2cc(Nc3ccnc(N4CC[C@@H](O)[C@@](C)(F)C4)n3)ncc12 ; C[C@@H]1CCN1c1ncc(CC2(C)COC2)c2cc(Nc3ccnc(N4CC[C@@H](O)[C@@](C)(F)C4)n3)ncc12]

Part 1: (3S,4R)-3-fluoro-3-methyl-1-(4-((8-(2-methylazetidin-1-yl)-5-((3-methyloxetan-3-yl)methyl)-2,7-naphthyridin-3-yl)amino)pyrimidin-2-yl)piperidin-4-ol was prepared from 6-chloro-1-(2-methylazetidin-1-yl)-4-((3-methyloxetan-3-yl)methyl)-2,7-naphthyridine (Preparation 286) and (3S,4R)-1-(4-aminopyrimidin-2-yl)-3-fluoro-3-methylpiperidin-4-ol (Preparation 8) using an analogous method to that described for Example 44 (BrettPhos Pd G3 method). Purified by prep-TLC (5:1 DCM/MeOH) followed by prep-HPLC-23 (Gradient (% organic) 34-44%) to give the title compound (70 mg, 50%).

Part 2: The compound of Part 1 was purified by chiral HPLC (CHIRALPAK IE; 20×250 mm, 5 mm; 30% MeOH/MTBE (+10 mM NH₃/MeOH) to afford the title compound as a white solid.

Peak 1: White solid (25 mg, 36%). LCMS m/z=508 [M+H]⁺¹HNMR (300 MHz, DMSO-d₆) δ: 10.03 (s, 1H), 9.03 (s, 1H), 8.25 (s, 1H), 8.04 (d, 1H, J=5.6 Hz), 7.87 (s, 1H), 6.63 (d, 1H, J=5.6 Hz), 5.04 (d, 1H, J=6.4 Hz), 4.78-4.66 (m, 4H), 4.56 (dd, 2H, J=5.7, 2.9 Hz), 4.22-4.08 (m, 3H), 3.58 (d, 1H, J=8.4 Hz), 3.17-3.06 (m, 4H), 2.07 (s, 1H), 1.45-1.43 (m, 2H), 1.39-1.26 (m, 10H).

### Example 157

### (S)-2-(7-((2-((3S,4R)-3-fluoro-4-hydroxy-3-methylpiperidin-1-yl)pyrimidin-4-yl)amino)-4-((R)-2-methylazetidin-1-yl)-2,6-naphthyridin-1-yl)propanenitrile or (R)-2-(7-((2-((3S,4R)-3-fluoro-4-hydroxy-3-methylpiperidin-1-yl)pyrimidin-4-yl)amino)-4-((R)-2-methylazetidin-1-yl)-2,6-naphthyridin-1-yl)propanenitrile

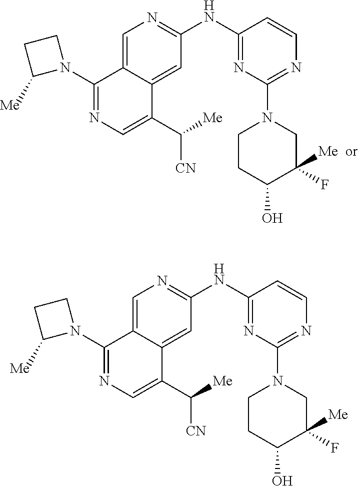 [SMILES: C[C@H](C#N)c1cnc(N2CC[C@H]2C)c2cnc(Nc3ccnc(N4CC[C@@H](O)[C@@](C)(F)C4)n3)cc12 ; C[C@@H]1CCN1c1ncc([C@@H](C)C#N)c2cc(Nc3ccnc(N4CC[C@@H](O)[C@@](C)(F)C4)n3)ncc12]

Part 1. 2-(3-((2-((3S,4R)-3-fluoro-4-hydroxy-3-methylpiperidin-1-yl)pyrimidin-4-yl)amino)-8-((R)-2-methylazetidin-1-yl)isoquinolin-5-yl)propanenitrile was prepared from 2-(7-chloro-4-((R)-2-methylazetidin-1-yl)-2,6-naphthyridin-1-yl)propanenitrile (Preparation 352) and (3S,4R)-1-(4-aminopyrimidin-2-yl)-3-fluoro-3-methylpiperidin-4-ol (Preparation 8) using an analogous method to that described for Example 44 (BrettPhos Pd G3 method). Purified by prep-TLC (20:1 DCM/MeOH).

Part 2: The compound of Part 1 was purified by chiral HPLC (CHIRALPAK IC; 20×250 mm, 5 mm; 20% EtOH/(3:1, Hex/DCM (+10 mM NH₃/MeOH)) to afford the title compound.

Peak 1: White solid (15 mg). LCMS m/z=477 [M+H]⁺¹HNMR (300 MHz, DMSO-d₆) δ:10.17 (s, 1H), 9.18 (s, 1H), 8.50 (s, 1H), 8.05 (d, 1H), 7.73 (s, 1H), 6.51 (d, 1H), 5.06 (d, 1H), 4.71 (dtd, 5H), 4.04 (q, 1H), 3.59 (dt, 1H), 3.26-3.02 (m, 2H), 2.56 (d, 1H), 2.17-1.99 (m, 1H), 1.84-1.63 (m, 5H), 1.48-1.32 (m, 6H).

### Example 158

### (3S,4R)-3-fluoro-1-(4-((5-((S)-1-hydroxypropan-2-yl)-8-((R)-2-methylazetidin-1-yl)isoquinolin-3-yl)amino)pyrimidin-2-yl)-3-methylpiperidin-4-ol or (3S,4R)-3-fluoro-1-(4-((5-((R)-1-hydroxypropan-2-yl)-8-((R)-2-methylazetidin-1-yl)isoquinolin-3-yl)amino)pyrimidin-2-yl)-3-methylpiperidin-4-ol

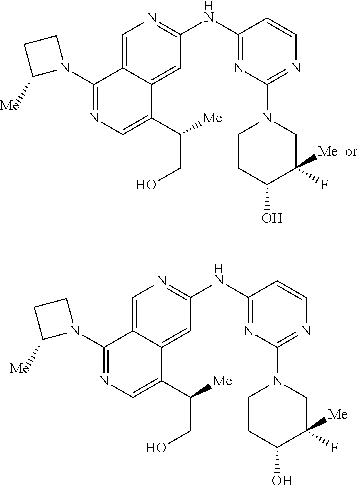 [SMILES: C[C@H](CO)c1cnc(N2CC[C@H]2C)c2cnc(Nc3ccnc(N4CC[C@@H](O)[C@@](C)(F)C4)n3)cc12 ; C[C@@H]1CCN1c1ncc([C@@H](C)CO)c2cc(Nc3ccnc(N4CC[C@@H](O)[C@@](C)(F)C4)n3)ncc12]

The title compound was prepared from 2-(3-chloro-8-((R)-2-methylazetidin-1-yl)isoquinolin-5-yl)propan-1-ol (Preparation 130) and (3S,4R)-1-(4-aminopyrimidin-2-yl)-3-fluoro-3-methylpiperidin-4-ol (Preparation 8) using an analogous method to that described for Example 44 (BrettPhos Pd G3 method). Purified by prep-HPLC-10 (Gradient (% organic): 40-50%) to afford the title compound.

Peak 2: Yellow solid (1 mg). LCMS m/z=481 [M+H]⁺¹HNMR (300 MHz, MeOH-d₄) δ: 9.12 (d, 1H), 8.58 (s, 1H), 7.96 (d, 1H), 7.46 (d, 1H), 6.64 (d, 1H), 6.40 (d, 1H), 4.78-4.67 (m, 2H), 4.51 (p, 1H), 4.43 (d, 1H) 3.89-3.57 (m, 5H), 3.31-3.15 (m, 2H) 2.51 (ddt, 11H), 2.23-2.10 (m, 1H), 1.93 (dt, 2H), 1.48 (s, 1H), 1.45-1.35 (m, 7H), 1.31 (d, 1).

### Example 159

### (3S,4R)-3-fluoro-1-(4-((5-isopropyl-8-((S)-2-methylazetidin-1-yl)-2,7-naphthyridin-3-yl)amino)pyrimidin-2-yl)piperidin-4-ol or (3S,4R)-3-fluoro-1-(4-((5-isopropyl-8-((R)-2-methylazetidin-1-yl)-2,7-naphthyridin-3-yl)amino)pyrimidin-2-yl)piperidin-4-ol

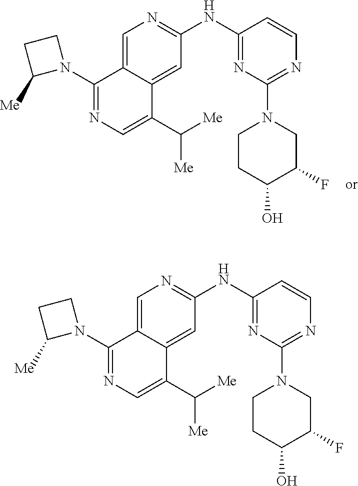 [SMILES: CC(C)c1cnc(N2CC[C@@H]2C)c2cnc(Nc3ccnc(N4CC[C@@H](O)[C@@H](F)C4)n3)cc12 ; CC(C)c1cnc(N2CC[C@H]2C)c2cnc(Nc3ccnc(N4CC[C@@H](O)[C@@H](F)C4)n3)cc12]

Part 1. (3S,4R)-3-fluoro-1-(4-((5-isopropyl-8-(2-methylazetidin-1-yl)-2,7-naphthyridin-3-yl)amino)pyrimidin-2-yl)piperidin-4-ol (280 mg, 85%) was prepared from 6-chloro-4-isopropyl-1-(2-methylazetidin-1-yl)-2,7-naphthyridine (Preparation 170) and (3S,4R)-1-(4-aminopyrimidin-2-yl)-3-fluoropiperidin-4-ol (Example A66 in WO2014/081718) using an analogous method to that described for Example 38 (RuPhos Pd G3 method). Prep-TLC (20:1 DCM/MeOH).

Part 2: The compound of Part 1 was purified by chiral HPLC (CHIRALPAK IC; 20×250 mm, 5 mm; 50% Hex (+8 mM NH₃/MeOH)/EtOH to afford the title compound as a yellow solid.

Peak 1: Yellow solid (50 mg, 36%). LCMS m/z=452 [M+H]⁺¹HNMR (300 MHz, DMSO-d₆) δ: 10.11 (s, 1H), 9.04 (s, 1H), 8.52 (s, 1H), 8.05 (d, 1H), 7.98 (s, 1H), 6.50 (d, 1H), 5.17 (d, 1H), 4.78 (d, 1H), 4.60 (t, 3H), 4.37 (d, 1H), 4.12 (q, 1H), 3.91 (s, 1H), 3.83 (d, 1H), 3.34 (dd, 3H), 2.08 (q, 1H), 1.73 (d, 2H), 1.44 (d, 3H), 1.32 (dd, 6H).

### Example 160

### (3R,4S)-3-fluoro-1-(4-((5-isopropyl-8-((S)-2-methylazetidin-1-yl)-2,7-naphthyridin-3-yl)amino)pyrimidin-2-yl)piperidin-4-ol or (3R,4S)-3-fluoro-1-(4-((5-isopropyl-8-((R)-2-methylazetidin-1-yl)-2,7-naphthyridin-3-yl)amino)pyrimidin-2-yl)piperidin-4-ol

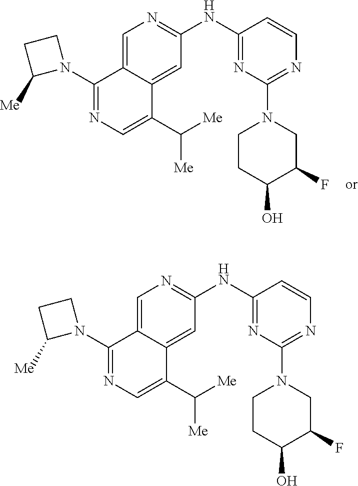 [SMILES: CC(C)c1cnc(N2CC[C@@H]2C)c2cnc(Nc3ccnc(N4CC[C@H](O)[C@H](F)C4)n3)cc12 ; CC(C)c1cnc(N2CC[C@H]2C)c2cnc(Nc3ccnc(N4CC[C@H](O)[C@H](F)C4)n3)cc12]

Part 1. (3R,4S)-3-fluoro-1-(4-((5-isopropyl-8-(2-methylazetidin-1-yl)-2,7-naphthyridin-3-yl)amino)pyrimidin-2-yl)piperidin-4-ol was prepared from 6-chloro-4-isopropyl-1-(2-methylazetidin-1-yl)-2,7-naphthyridine (Preparation 170) and (3R,4S)-1-(4-aminopyrimidin-2-yl)-3-fluoropiperidin-4-ol (Example A64 in WO2014/081718) using an analogous method to that described for Example 38 (RuPhos Pd G3 method). Prep-TLC (30:1 DCM/MeOH).

Part 2: The compound of Part 1 was purified by chiral HPLC (CHIRALPAK IC; 20×250 mm, 5 mm; 50% Hex (+8 mM NH₃/MeOH)/EtOH to afford the title compound as a yellow solid.

Peak 2: Yellow solid (120 mg, 36%). LCMS m/z=452 [M+H]⁺¹HNMR (300 MHz, DMSO-d₆) δ: 10.09 (s, 1H), 9.03 (s, 1H), 8.52 (s, 1H), 8.08-7.96 (m, 2H), 6.50 (d, 1H), 5.17 (d, 1H), 4.77 (dd, 1H), 4.71-4.54 (m, 3H), 4.11 (q, 1H), 3.96-3.88 (m, 1H), 3.83 (d, 1H), 3.61 (d, 1H), 3.30 (d, 3H), 2.07 (s, 1H), 1.73 (d, 2H), 1.43 (d, 3H), 1.32 (dd, 6H).

### Example 161

### (3S,4R)-3-fluoro-1-(4-((5-(2-fluoropyridin-3-yl)-8-((R)-2-methylazetidin-1-yl)-2,7-naphthyridin-3-yl)amino)pyrimidin-2-yl)piperidin-4-ol or (3S,4R)-3-fluoro-1-(4-((5-(2-fluoropyridin-3-yl)-8-((S)-2-methylazetidin-1-yl)-2,7-naphthyridin-3-yl)amino)pyrimidin-2-yl)piperidin-4-ol

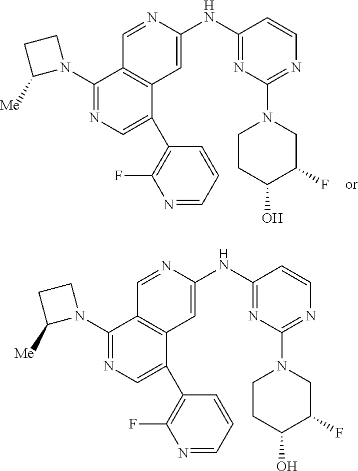 [SMILES: C[C@@H]1CCN1c1ncc(-c2cccnc2F)c2cc(Nc3ccnc(N4CC[C@@H](O)[C@@H](F)C4)n3)ncc12 ; C[C@H]1CCN1c1ncc(-c2cccnc2F)c2cc(Nc3ccnc(N4CC[C@@H](O)[C@@H](F)C4)n3)ncc12]

Part 1. (3S,4R)-3-fluoro-1-(4-((5-(2-fluoropyridin-3-yl)-8-(2-methylazetidin-1-yl)-2,7-naphthyridin-3-yl)amino)pyrimidin-2-yl)piperidin-4-ol was prepared from 6-chloro-4-(2-fluoropyridin-3-yl)-1-(2-methylazetidin-1-yl)-2,7-naphthyridine (Preparation 306) and (3S,4R)-1-(4-aminopyrimidin-2-yl)-3-fluoropiperidin-4-ol (Example A66 in WO2014/081718) using an analogous method to that described for Example 1 using XPhos Pd G4 as catalyst. Prep-TLC (30:1 DCM/MeOH).

Part 2: The compound of Part 1 was purified by chiral HPLC (CHIRALPAK IE; 20×250 mm, 5 mm; 5% EtOH/MTBE (10 mM NH₃/MeOH) to afford the title compound as a white solid.

Peak 1: White solid (19 mg). LCMS m/z=505 [M+H]⁺¹HNMR (400 MHz, DMSO-d₆) δ:10.06 (s, 1H), 9.10 (s, 1H), 8.35 (ddd, 1H), 8.18-7.90 (m, 3H), 7.88-7.77 (m, 1H), 7.55 (ddd, 1H), 6.54 (d, 1H), 5.06 (d, 1H), 4.82 (dtd, 2H), 4.67-4.13 (m, 2H), 3.93 (s, 1H), 3.69 (dd, 1H), 3.02 (s, 1H), 2.89-2.53 (m, 1H), 2.10 (td, 2H), 1.69-1.33 (m, 6H).

### Example 162

### (3S,4R)-3-fluoro-1-(4-((8-((R)-2-methylazetidin-1-yl)-5-((S)-tetrahydrofuran-3-yl)-2,7-naphthyridin-3-yl)amino)pyrimidin-2-yl)piperidin-4-ol or (3S,4R)-3-fluoro-1-(4-((8-((R)-2-methylazetidin-1-yl)-5-((R)-tetrahydrofuran-3-yl)-2,7-naphthyridin-3-yl)amino)pyrimidin-2-yl)piperidin-4-ol

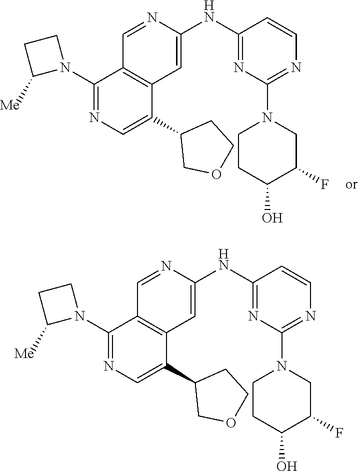 [SMILES: C[C@@H]1CCN1c1ncc([C@@H]2CCOC2)c2cc(Nc3ccnc(N4CC[C@@H](O)[C@@H](F)C4)n3)ncc12 ; C[C@@H]1CCN1c1ncc([C@H]2CCOC2)c2cc(Nc3ccnc(N4CC[C@@H](O)[C@@H](F)C4)n3)ncc12]

Part 1. A mixture of (R)-6-chloro-4-(4,5-dihydrofuran-3-yl)-1-(2-methylazetidin-1-yl)-2,7-naphthyridine (Preparation 320, 58 mg, 0.192 mmol), (3S,4R)-1-(4-aminopyrimidin-2-yl)-3-fluoropiperidin-4-ol (Example A66 in WO2014/081718, 41 mg, 0.192 mmol), XPhos Pd G4 (8.3 mg, 0.0096 mmol) and Cs₂CO₃ (125 mg, 0.384 mmol) in dioxane (1 mL) was heated under N₂ at 90° C. for 1 h. The reaction was diluted with DCM and washed with H2O. The combined organics were dried (Na₂SO₄) and evaporated to dryness in vacuo. The residue was purified by ISCO-chromatography (0-10% MeOH/DCM) to afford (3S,4R)-1-(4-((5-(4,5-dihydrofuran-3-yl)-8-((R)-2-methylazetidin-1-yl)-2,7-naphthyridin-3-yl)amino)pyrimidin-2-yl)-3-fluoropiperidin-4-ol as a yellow foam (79 mg, 86%).

Part 2. The compound of Part 1 (74 mg, 0.155 mmol) in MeOH (3 mL) was hydrogenated at 60 psi H₂ at 50° C. for 3 days in the presence of 10% Pd/C (10%). The catalyst was removed by filtration through a pad of Celite and the filtrate evaporated to dryness to afford (3S,4R)-3-fluoro-1-(4-((8-((R)-2-methylazetidin-1-yl)-5-(tetrahydrofuran-3-yl)-2,7-naphthyridin-3-yl)amino)pyrimidin-2-yl)piperidin-4-ol as an off-white solid (75 mg).

Part 3. The compound of Part 2 was purified by prep-HPLC (CHIRALPAK IC-3; 4.6×50 mm, 3 mm; 50% (3:1 Hex:DCM)/IPA (+0.1% DEA)) to afford the title compound as a white solid.

Peak 2: LCMS m/z=480 [M+H]⁺¹HNMR (400 MHz, DMSO-d₆) δ: 10.07 (s, 1H), 9.03 (s, 1H), 8.44 (s, 1H), 8.07-7.99 (m, 2H), 6.54 (d, 1H), 5.13 (d, 1H), 4.78 (dd, 2H), 4.70-4.55 (m, 3H), 4.38 (d, 1H), 4.17-4.05 (m, 2H), 3.97 (td, 1H), 3.92-3.76 (m, 2H), 3.70 (q, 1H), 3.68-3.60 (m, 1H), 3.63-3.45 (m, 1H), 2.32 (s, 1H), 2.17-2.00 (m, 2H), 1.71 (d, 2H), 1.44 (d, 3H), 1.23 (s, 1H).

### Example 163

### 8-((2R,3S)-3-(2-aminoethoxy)-2-methylazetidin-1-yl)-N-(2-((3S,4R)-3-fluoro-4-methoxypiperidin-1-yl)pyrimidin-4-yl)-5-isopropyl-2,7-naphthyridin-3-amine

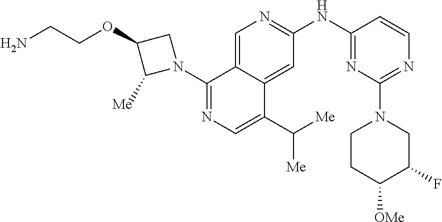 [SMILES: CO[C@@H]1CCN(c2nccc(Nc3cc4c(C(C)C)cnc(N5C[C@H](OCCN)[C@H]5C)c4cn3)n2)C[C@@H]1F]

Part 1. MsCl (84.8 mg, 0.741 mmol) was added to a solution of 2-(((2R,3S)-1-(6-((2-((3S,4R)-3-fluoro-4-methoxypiperidin-1-yl)pyrimidin-4-yl)amino)-4-isopropyl-2,7-naphthyridin-1-yl)-2-methylazetidin-3-yl)oxy)ethan-1-ol (Example 148, 300 mg, 0.57 mmol) and TEA (86.5 mg, 0.855 mmol) in DCM and stirred at rt for 2 h. The solution was evaporated to dryness in vacuo to afford 2-(((2R,3S)-1-(6-((2-((3S,4R)-3-fluoro-4-hydroxypiperidin-1-yl)pyrimidin-4-yl)amino)-4-isopropyl-2,7-naphthyridin-1-yl)-2-methylazetidin-3-yl)oxy)ethyl methanesulfonate which was used without further purification.

Part 2. A solution of NH₃ in MeOH (7 mM, 3 mL) was added to a solution of 2-(((2R,3S)-1-(6-((2-((3S,4R)-3-fluoro-4-hydroxypiperidin-1-yl)pyrimidin-4-yl)amino)-4-isopropyl-2,7-naphthyridin-1-yl)-2-methylazetidin-3-yl)oxy)ethyl methanesulfonate (Part 1, 230 mg, 0.38 mmol) in MeOH (1 mL) and stirred for 2 h in a sealed vial at 100° C. The reaction mixture was evaporated to dryness in vacuo and the residue purified by prep-HPLC-21 (Gradient (% organic) 32-40%) to afford the title compound as a white solid (78.2 mg, 26%). LCMS m/z=525 [M+H]⁺¹HNMR (300 MHz, DMSO-d₆) δ: 10.11 (s, 1H), 9.06 (s, 1H), 8.52 (s, 1H), 8.05 (d, 1H), 8.01 (s, 1H), 6.53 (d, 1H), 5.11-4.79 (m, 2H), 4.73 (dd, 1H), 4.60-4.36 (m, 2H), 4.09 (q, 1H), 3.88 (dd, 1H), 3.67-3.33 (m, 11H), 2.68 (t, 2H), 1.88-1.62 (m, 2H), 1.46 (d, 3H), 1.33 (dd, 6H).

### Example 164-165

The title compounds were prepared from 2-((3R,4S)-3-fluoro-4-methoxypiperidin-1-yl)pyrimidin-4-amine (Preparation 32) and the appropriate chloride (RCl) in an analogous 2-step process as described for Example 39.

| Ex no | Name/Structure/RNH₂/HPLC/Data |
|---|---|
| 164 | N-(2-((3R,4S)-3-fluoro-4-methoxypiperidin-1-yl)pyrimidin-4-yl)-5-isopropyl-8- |
| | ((S)-2-((methylamino)methyl)azetidin-1-yl)-2,7-naphthyridin-3-amine |
| | 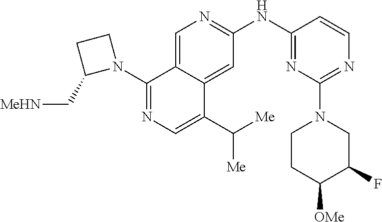 [SMILES: CNC[C@@H]1CCN1c1ncc(C(C)C)c2cc(Nc3ccnc(N4CC[C@H](OC)[C@H](F)C4)n3)ncc12] |
| | RCl: tert-butyl (S)-((1-(6-chloro-4-isopropyl-2,7-naphthyridin-1-yl)azetidin-2- |
| | yl)methyl)(methyl)carbamate (Preparation 231). |
| | HPLC-21, Gradient (% organic): 30-42% |
| | 30 mg, white solid. LCMS m/z = 494 [M + H]⁺ ¹HNMR (300 MHz, DMSO-d₆) δ: |
| | 10.11 (s, 1H), 9.05 (s, 1H), 8.50 (s, 1H), 8.05 (d, 1H), 7.97 (s, 1H), 6.52 (d, 1H), 5.03 (s, 1H), |
| | 4.90-4.75 (m, 1H), 4.75-4.57 (m, 2H), 4.48 (d, 1H), 4.15 (q, 1H), 3.65 (d, 2H), 3.58-3.42 (m, |
| | 6H), 3.00-2.79 (m, 2H), 2.36 (s, 5H), 1.83 (t, 1H), 1.73 (d, 1H), 1.32 (dd, 6H). |
| 165 | 8-((S)-2-(aminomethyl)azetidin-1-yl)-N-(2-((3R,4S)-3-fluoro-4- |
| | methoxypiperidin-1-yl)pyrimidin-4-yl)-5-isopropyl-2,7-naphthyridin-3- |
| | amine |
| | 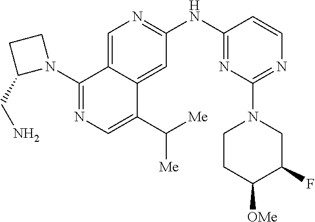 [SMILES: CO[C@H]1CCN(c2nccc(Nc3cc4c(C(C)C)cnc(N5CC[C@H]5CN)c4cn3)n2)C[C@H]1F] |
| | RCl: tert-butyl (S)-((1-(6-chloro-4-isopropyl-2,7-naphthyridin-1-yl)azetidin-2- |
| | yl)methyl)carbamate (Preparation 171) |
| | HPLC-21, Gradient (% organic): 30-42% |
| | 40 mg, white solid. LCMS m/z = 480 [M + H]⁺ ¹HNMR (300 MHz, DMSO-d₆) δ: |
| | 10.11 (s, 1H), 9.05 (s, 1H), 8.49 (s, 1H), 8.05 (d, 1H), 7.97 (s, 1H), 6.52 (d, 1H), 5.03 (s, 1H), |
| | 4.87 (s, 1H), 4.71 (d, 1H), 4.63 (d, 1H), 4.48 (d, 1H), 4.15 (q, 1H), 3.53 (d, 2H), 3.45 (s, 4H), |
| | 3.37 (s, 3H), 2.93 (d, 2H), 2.35 (s, 2H), 1.90-1.68 (m, 2H), 1.32 (dd, 6H). |

### Example 166

### (3S,4R)-1-(4-((5-isopropyl-8-((S)-2-methylazetidin-1-yl)-2,7-naphthyridin-3-yl)amino)pyrimidin-2-yl)-4-methoxypiperidin-3-ol or (3R,4S)-1-(4-((5-isopropyl-8-((S)-2-methylazetidin-1-yl)-2,7-naphthyridin-3-yl)amino)pyrimidin-2-yl)-4-methoxypiperidin-3-ol or (3R,4S)-1-(4-((5-isopropyl-8-((S)-2-methylazetidin-1-yl)-2,7-naphthyridin-3-yl)amino)pyrimidin-2-yl)-4-methoxypiperidin-3-ol or (3R,4S)-1-(4-((5-isopropyl-8-((R)-2-methylazetidin-1-yl)-2,7-naphthyridin-3-yl)amino)pyrimidin-2-yl)-4-methoxypiperidin-3-ol

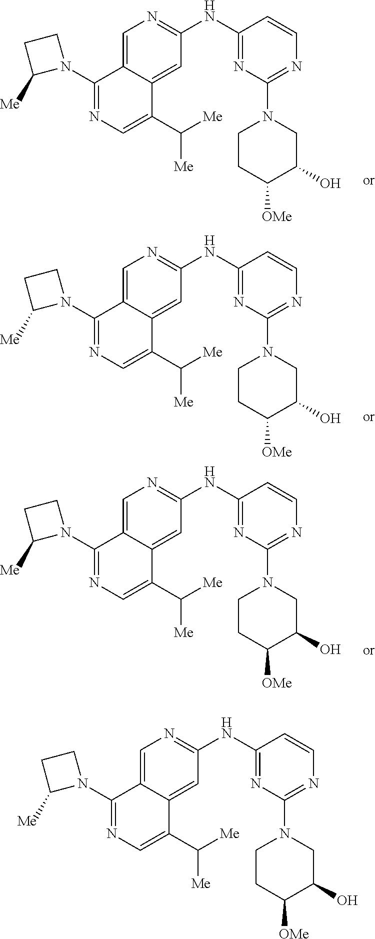

A mixture of 6-chloro-4-isopropyl-1-(2-methylazetidin-1-yl)-2,7-naphthyridine (Preparation 170, 160 mg, 0.580 mmol), (3S,4R)-1-(4-aminopyrimidin-2-yl)-4-methoxypiperidin-3-ol or (3R,4S)-1-(4-aminopyrimidin-2-yl)-4-methoxypiperidin-3-ol (The compound of Peak 1 of Preparation 16 and 17, 130 mg, 0.580 mmol), Cs₂CO₃ (377 mg, 1.16 mmol) and RuPhos Pd G3 (97.0 mg, 0.116 mmol) in dioxane (4 mL) was stirred under N₂ at 100° C. for 16 h. The reaction was evaporated to dryness and the residue purified by column chromatography (20:1, DCM/MeOH) and then further purified by prep-HPLC (ChiralPak IG, 50×250 mm, 5 mm; 50% EtOH/Hex (+8 mM NH3/MeOH) to afford:

Peak 1 as a yellow solid (50 mg). LCMS m/z=463 [M+H]⁺; ¹HNMR (300 MHz, DMSO-d₆) δ:10.03 (s, 1H), 9.02 (s, 1H), 8.54 (s, 1H), 8.01 (d, 1H), 7.97 (s, 1H), 6.45 (d, 1H), 4.77 (q, 1H), 4.64 (t, 2H), 4.13-4.04 (m, 1H), 3.96 (s, 2H), 3.69 (d, 3H), 3.48 (s, 2H), 3.45 (d, 4H), 2.07 (d), 1.86 (s, 1H), 1.62 (s, 1H), 1.42 (d, 3H), 1.36-1.27 (m, 6H).

### Example 167

### 2-(((3R,4S)-1-(4-((5-isopropyl-8-((R)-2-methylazetidin-1-yl)-2,7-naphthyridin-3-yl)amino)pyrimidin-2-yl)-4-methoxypiperidin-3-yl)oxy)ethan-1-ol or 2-(((3S,4R)-1-(4-((5-isopropyl-8-((R)-2-methylazetidin-1-yl)-2,7-naphthyridin-3-yl)amino)pyrimidin-2-yl)-4-methoxypiperidin-3-yl)oxy)ethan-1-ol

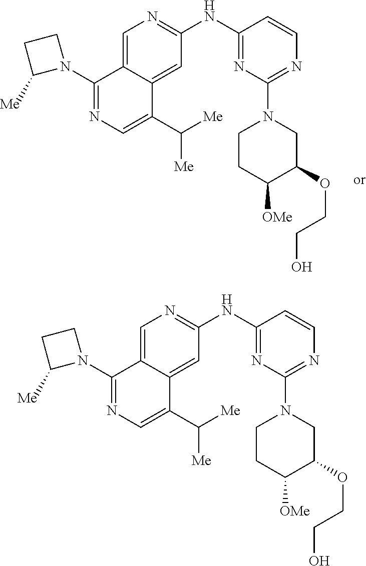

A mixture of (R)-6-chloro-4-isopropyl-1-(2-methylazetidin-1-yl)-2,7-naphthyridine (Preparation 191, 40 mg, 0.145 mmol), Cis-rac-2-((1-(4-aminopyrimidin-2-yl)-4-methoxypiperidin-3-yl)oxy)ethan-1-ol (Preparation 56, 38.9 mg, 0.145 mmol), Cs₂CO₃ (70.7 mg, 0.217 mmol) and BrettPhos Pd G3 (13.1 mg, 0.015 mmol) in dioxane (2 mL) was stirred under N₂ at 100° C. for 16 h. The reaction mixture was diluted with EtOAc and washed with brine (×2). The combined organics were dried (Na₂SO₄) and evaporated to dryness and the residue purified by prep-TLC (20:1, DCM/MeOH) and then further purified by prep-HPLC (ChiralPak IA, 20×250 mm, 5 mm; 20% IPA/Hex (+8 mM NH₃/MeOH) to afford the title compound.

Peak 1 as a white solid (5.5 mg). LCMS m/z=508 [M+H]*; ¹HNMR (300 MHz, DMSO-d₆) δ: 10.02 (s, 1H), 9.02 (s, 1H), 8.48 (s, 1H), 8.03 (d, 1H), 7.98 (s, 1H), 6.51 (d, 1H), 4.78 (q, 1H), 4.63 (d, 1H), 4.48 (s, 1H), 4.11 (q, 2H), 3.97 (s, 1H), 3.71 (d, 2H), 3.63-3.58 (m, 2H), 3.56-3.45 (m, 2H), 3.42 (d, 2H), 3.35-3.33 (m, 2H), 3.29 (s, 3H), 2.06 (p, 1H), 1.86 (d, 1H), 1.64 (s, 1H), 1.43 (d, 3H), 1.31 (d, 6H).

### Example 168

### (R)-1-(4-((8-((2R,3S)-3-hydroxy-2-methylazetidin-1-yl)-5-isopropyl-2,7-naphthyridin-3-yl)amino)pyrimidin-2-yl)-3,3-dimethylpiperidin-4-ol or (S)-1-(4-((8-((2R,3S)-3-hydroxy-2-methylazetidin-1-yl)-5-isopropyl-2,7-naphthyridin-3-yl)amino)pyrimidin-2-yl)-3,3-dimethylpiperidin-4-ol

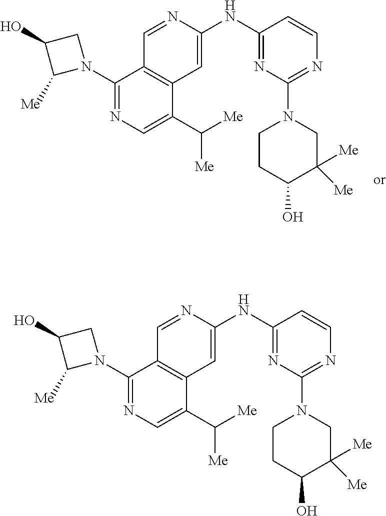 [SMILES: CC(C)c1cnc(N2C[C@H](O)[C@H]2C)c2cnc(Nc3ccnc(N4CC[C@@H](O)C(C)(C)C4)n3)cc12 ; CC(C)c1cnc(N2C[C@H](O)[C@H]2C)c2cnc(Nc3ccnc(N4CC[C@H](O)C(C)(C)C4)n3)cc12]

A mixture of (2R,3S)-1-(6-chloro-4-isopropyl-2,7-naphthyridin-1-yl)-2-methylazetidin-3-ol (Preparation 213, 150 mg, 0.514 mmol), 1-(4-aminopyrimidin-2-yl)-3,3-dimethylpiperidin-4-ol (Preparation 57, 114 mg, 0.514 mmol), Cs₂CO₃ (332 mg, 1.02 mmol), RuPhos Pd G3 (42.9 mg, 0.051 mmol) and BINAP Pd G2 (47.9 mg, 0.051 mmol) in dry dioxane was stirred under N₂ at 100° C. for 2 h. The reaction mixture was evaporated to dryness and the residue purified by prep-HPLC-12 (Gradient (% organic), 35-59%) and further separated by chiral-HPLC (CHIRALPAK IG-3 4.6×50 mm, 3 mm; 75% IPA/Hex (0.1% DEA) to afford the title compound.

Peak 2 as a white solid (8.4 mg). LCMS m/z=478 [M+H]⁺; ¹HNMR (300 MHz, DMSO-d₆) δ: 10.01 (s, 1H), 9.03 (s, 1H), 8.50 (s, 1H), 8.03-7.95 (m, 2H), 6.42 (d, 1H), 5.59 (d, 1H), 4.84 (t, 1H), 4.68 (d, 1H), 4.38 (s, 2H), 4.18-4.05 (m, 2H), 3.74 (s, 1H), 3.39 (s, 2H), 3.34 (s, 1H), 2.98 (d, 1H), 1.71 (m, 1H), 1.39 (d, 4H), 1.29 (dd, 6H), 0.91 (s, 3H), 0.78 (s, 3H).

### Example 169

### (R)-5-(4-((8-((2R,3S)-3-hydroxy-2-methylazetidin-1-yl)-5-isopropyl-2,7-naphthyridin-3-yl)amino)pyrimidin-2-yl)-5-azaspiro[2.5]octan-8-ol or (S)-5-(4-((8-((2R,3S)-3-hydroxy-2-methylazetidin-1-yl)-5-isopropyl-2,7-naphthyridin-3-yl)amino)pyrimidin-2-yl)-5-azaspiro[2.5]octan-8-ol

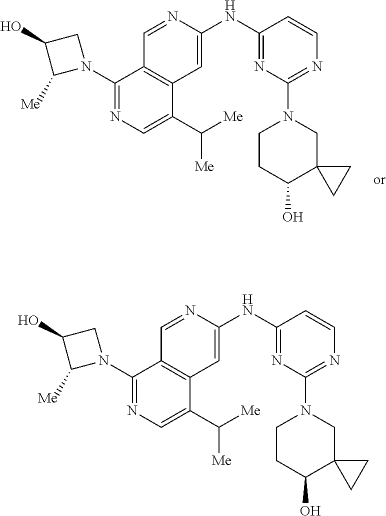 [SMILES: CC(C)c1cnc(N2C[C@H](O)[C@H]2C)c2cnc(Nc3ccnc(N4CC[C@@H](O)C5(CC5)C4)n3)cc12 ; CC(C)c1cnc(N2C[C@H](O)[C@H]2C)c2cnc(Nc3ccnc(N4CC[C@H](O)C5(CC5)C4)n3)cc12]

The title compound was prepared from (2R,3S)-1-(6-chloro-4-isopropyl-2,7-naphthyridin-1-yl)-2-methylazetidin-3-ol (Preparation 213) and 5-(4-aminopyrimidin-2-yl)-5-azaspiro[2.5]octan-8-ol (Preparation 60) in an analogous manner as described for Example 168. Chiral-HPLC (CHIRALPAK AD 4.6×100 mm, 5 mm; 20% IPA/Hex (0.1% DEA) to afford the title compound.

Peak 2 as a white solid (8 mg, 26%). LCMS m/z=476 [M+H]⁺; ¹HNMR (400 MHz, DMSO-d₆) δ: 10.06 (s, 1H), 9.05 (s, 1H), 8.49 (s, 1H), 8.01 (d, 2H), 6.46 (d, 1H), 5.62 (d, 1H), 4.86 (d, 1H), 4.74 (d, 1H), 4.40 (s, 1H), 4.17 (p, 1H), 4.04-3.90 (m, 2H), 3.84-3.74 (m, 2H), 3.56 (d, 1H), 1.90-1.77 (m, 1H), 1.66-1.54 (m, 1H), 1.42 (d, 3H), 1.32 (dd, 6H), 0.58-0.42 (m, 2H), 0.39-0.22 (m, 2H).

### Example 170

### (3R,4S)-3-fluoro-1-(4-((8-((2R,3S)-3-hydroxy-2-methylazetidin-1-yl)-5-isopropyl-2,7-naphthyridin-3-yl)amino)pyrimidin-2-yl)-3,4-dimethylpiperidin-4-ol or (3S,4R)-3-fluoro-1-(4-((8-((2R,3S)-3-hydroxy-2-methylazetidin-1-yl)-5-isopropyl-2,7-naphthyridin-3-yl)amino)pyrimidin-2-yl)-3,4-dimethylpiperidin-4-ol

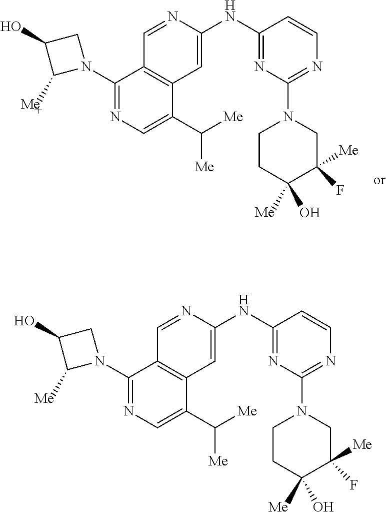 [SMILES: CC(C)c1cnc(N2C[C@H](O)[C@H]2C)c2cnc(Nc3ccnc(N4CC[C@](C)(O)[C@](C)(F)C4)n3)cc12 ; CC(C)c1cnc(N2C[C@H](O)[C@H]2C)c2cnc(Nc3ccnc(N4CC[C@@](C)(O)[C@@](C)(F)C4)n3)cc12]

The title compound was prepared from rac-cis-1-(4-aminopyrimidin-2-yl)-3-fluoro-3,4-dimethylpiperidin-4-ol (Preparation 37) and (2R,3S)-1-(6-chloro-4-isopropyl-2,7-naphthyridin-1-yl)-2-methylazetidin-3-ol (Preparation 213) in an analogous manner as described for Example 168. chiral-HPLC (CHIRALPAK IC, 20×250 mm, 5 mm; 50% EtOH/Hex (8 mM NH₃/MeOH) to afford the title compound.

Peak 2 as a white solid (31 mg, 38%). LCMS m/z=496 [M+H]⁺; ¹HNMR (400 MHz, DMSO-d₆) δ: 10.08 (s, 1H), 9.06 (s, 1H), 8.51 (s, 1H), 8.15-7.91 (m, 2H), 6.51 (d, 1H), 5.61 (d, 1H), 4.86 (t, 1H), 4.74 (s, 1H), 4.50-3.98 (m, 4H), 3.73 (dd, 3H), 3.343 (m, 1H) 1.85 (s, 1H), 1.57 (s, 1H), 1.50-1.15 (m, 15H).

### Example 171

### (S)-3,3-difluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol or (R)-3,3-difluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol

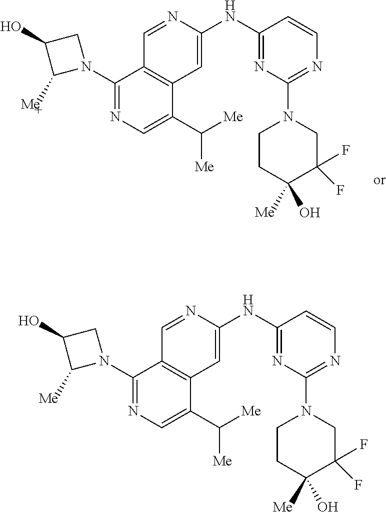 [SMILES: CC(C)c1cnc(N2C[C@H](O)[C@H]2C)c2cnc(Nc3ccnc(N4CC[C@](C)(O)C(F)(F)C4)n3)cc12 ; CC(C)c1cnc(N2C[C@H](O)[C@H]2C)c2cnc(Nc3ccnc(N4CC[C@@](C)(O)C(F)(F)C4)n3)cc12]

The title compound was prepared from Rac-1-(4-aminopyrimidin-2-yl)-3,3-difluoro-4-methylpiperidin-4-ol (Preparation 52) and (2R,3S)-1-(6-chloro-4-isopropyl-2,7-naphthyridin-1-yl)-2-methylazetidin-3-ol (Preparation 213) in an analogous manner as described for Example 168. chiral-HPLC (CHIRALPAK IE, 20×250 mm, 5 mm; 20% EtOH/Hex (+0.1% DEA) to afford the title compound.

Peak 2 as a white solid (33.5 mg, 8%). LCMS m/z=500 [M+H]⁺; ¹HNMR (300 MHz, DMSO-d₆) δ: 10.13 s, 1H), 9.04 (s, 1H), 8.47 (s, 1H), 8.27-7.72 (i, 2H), 6.54 (d, 1H), 5.99-5.45 (m, 2H), 5.12-4.55 (m, 21), 4.57-4.30 (m, 2H), 4.15 (t, 1H), 3.91-3.46 (m, 2H), 3.46-3.35 (m, 21), 1.71 (s, 2H), 1.55-1.07 (m, 12H).

### Example 172 and 173

### (3S,4R)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol and (3R,4S)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol

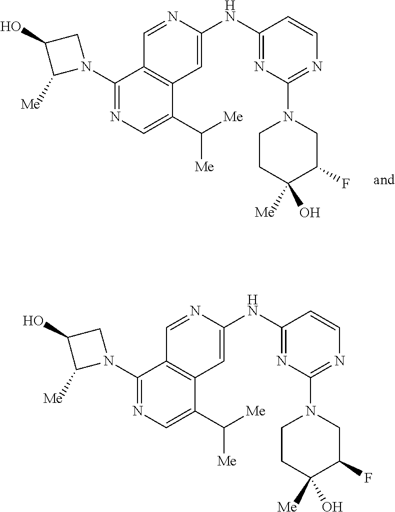 [SMILES: CC(C)c1cnc(N2C[C@H](O)[C@H]2C)c2cnc(Nc3ccnc(N4CC[C@](C)(O)[C@@H](F)C4)n3)cc12 ; CC(C)c1cnc(N2C[C@H](O)[C@H]2C)c2cnc(Nc3ccnc(N4CC[C@@](C)(O)[C@H](F)C4)n3)cc12]

A solution of (2R,3S)-1-(6-chloro-4-isopropyl-2,7-naphthyridin-1-yl)-2-methylazetidin-3-ol (Preparation 213, 120 mg, 0.411 mmol), cis-rac-1-(4-aminopyrimidin-2-yl)-3-fluoro-4-methylpiperidin-4-ol (Preparation 12, 92.9 mg, 0.411 mmol), Cs₂CO₃ (269 mg, 0.811 mmol) and Brettphos Pd G3 (37.2 mg, 0.041 mmol) in dioxane (3 ml) was heated to 120° C. under N₂ for 5 h. The reaction was evaporated to dryness in vacuo and the residue was purified by preparative TLC (10:1 DCM/MeOH) to give 3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol as a pale yellow solid (60 mg). The residue was further purified by chiral-HPLC (CHIRALPAK IC, 20×250 mm, 5 mm; 30% EtOH/Hex (8 8 mM NH₃/MeOH)— to afford the title compounds.

Peak 1 (Example 172) (3S,4R)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol or (3R,4S)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol, pale yellow solid (12.1 mg): LCMS m/z=482 [M+H]⁺; ¹HNMR (400 MHz, DMSO-d₆) δ: 10.13 (s, 1H), 9.06 (s, 1H), 8.54 (s, 1H), 8.06 (d, 1H), 8.01 (s, 1H), 6.51 (d, 1H), 5.61 (d, 1H), 4.87 (d, 2H), 4.41 (dd, 3H), 4.21-4.13 (m, 2H), 3.77 (dd, 1H), 3.62 (s, 1H), 3.49 (t, 1H), 3.31 (d, 1H), 1.73 (d, 1H), 1.58 (d, 1H), 1.42 (d, 3H), 1.35-1.22 (m, 9H).

Peak 2 (Example 173) (3S,4R)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol or (3R,4S)-3-fluoro-1-(4-((8-((2R,3S)-3-hydroxy-2-methylazetidin-1-yl)-5-isopropyl-2,7-naphthyridin-3-yl)amino)pyrimidin-2-yl)-4-methylpiperidin-4-ol, pale yellow solid (12.3 mg): LCMS m/z=482 [M+H]⁺; ¹HNMR (400 MHz, DMSO-d₆) δ: 10.13 (s, 1H), 9.06 (s, 1H), 8.54 (s, 1H), 8.06 (d, 1H), 8.01 (s, 1H), 6.51 (d, 1H), 5.61 (d, 1H), 4.87 (d, 2H), 4.41 (dd, 3H), 4.21-4.13 (m, 2H), 3.77 (dd, 1H), 3.62 (s, 1H), 3.49 (t, 1H), 3.31 (d, 1H), 1.73 (d, 1H), 1.58 (d, 1H), 1.42 (d, 3H), 1.35-1.22 (m, 9H).

### Example 174 and 175

### 6-(6-((2-((3S,4R)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide and 6-(6-((2-((3R,4S)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide

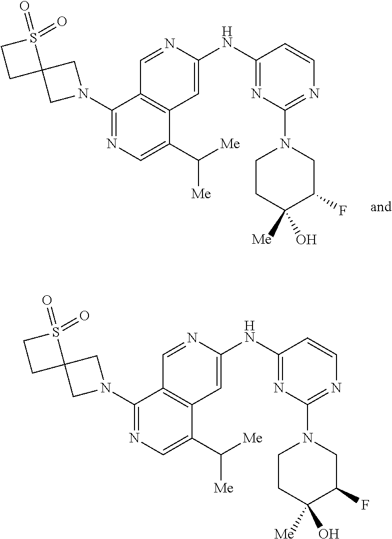 [SMILES: CC(C)c1cnc(N2CC3(CCS3(=O)=O)C2)c2cnc(Nc3ccnc(N4CC[C@@](C)(O)[C@@H](F)C4)n3)cc12 ; CC(C)c1cnc(N2CC3(CCS3(=O)=O)C2)c2cnc(Nc3ccnc(N4CC[C@](C)(O)[C@H](F)C4)n3)cc12]

A solution of 6-(6-chloro-4-isopropyl-2,7-naphthyridin-1-yl)-1-thia-6-azaspiro[3.3]heptane 1,1-dioxide (Preparation 217, 150 mg, 0.426 mmol), cis-rac-1-(4-aminopyrimidin-2-yl)-3-fluoro-4-methylpiperidin-4-ol (Preparation 12, 96.4 mg, 0.426 mmol), Cs₂CO₃ (414 mg, 1.27 mmol) and XantPhos Pd G2 (37.8 mg, 0.042 mmol) in dioxane (3 ml) was heated to 100° C. under N₂ for 2 h. The reaction was diluted with H₂O (20 mL) and extracted with EtOAc (2×20 mL). The combined extracts were dried (Na₂SO₄) and evaporated to dryness in vacuo and the residue was purified by prep-HPLC-23 (Gradient (% organic) 33-43%) to give 6-(6-((2-(3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide as a white solid (80 mg, 34%). The residue was further purified by prep-HPLC (CHIRALPAK IC, 20×250 mm, 5 mm; 50% EtOH/Hex (10 mM NH₃/MeOH) to afford the title compounds.

Peak 1 (Example 174) 6-(6-((2-((3S,4R)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide or 6-(6-((2-((3R,4S)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide, white solid (30 mg): LCMS m/z=542 [M+H]*; ¹HNMR (300 MHz, DMSO-d₆) δ: 10.23 (s, 1H), 9.06 (s, 1H), 8.59 (s, 1H), 8.19-8.01 (m, 2H), 6.57-6.43 (m, 1H), 4.88 (s, 1H), 4.78 (d, 2H), 4.64 (d, 2H), 4.44 (s, 2H), 4.34-4.06 (m, 4H), 3.72-3.43 (m, 2H), 2.44 (d, 2H), 1.80-1.50 (m, 2H), 1.40-1.19 (m, 9H).

Peak 2 (Example 175) 6-(6-((2-((3S,4R)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide or 6-(6-((2-((3R,4S)-3-fluoro-4-hydroxy-4-methylpiperidin-1-yl)pyrimidin-4-yl)amino)-4-isopropyl-2,7-naphthyridin-1-yl)-1-thia-6-azaspiro[3.3]heptane 1,1-dioxide, white solid (30 mg): LCMS m/z=542 [M+H]⁺; ¹HNMR (300 MHz, DMSO-d₆) δ: 10.23 (s, 1H), 9.06 (s, 1H), 8.59 (s, 1H), 8.19-8.01 (m, 2H), 6.57-6.43 (m, 1H), 4.88 (s, 1H), 4.78 (d, 2H), 4.64 (d, 2H), 4.44 (s, 2H), 4.34-4.06 (m, 4H), 3.72-3.43 (m, 2H), 2.44 (d, 2H), 1.80-1.50 (m, 2H), 1.40-1.19 (m, 9H).

### Example 176 and 177

### (3S,4R)-3-fluoro-3-methyl-1-(4-((8-((R)-2-methylazetidin-1-yl)-5-((S)-1-(oxetan-3-yl)ethyl)-2,7-naphthyridin-3-yl)amino)pyrimidin-2-yl)piperidin-4-ol and (3S,4R)-3-fluoro-3-methyl-1-(4-((8-((R)-2-methylazetidin-1-yl)-5-((R)-1-(oxetan-3-yl)ethyl)-2,7-naphthyridin-3-yl)amino)pyrimidin-2-yl)piperidin-4-ol

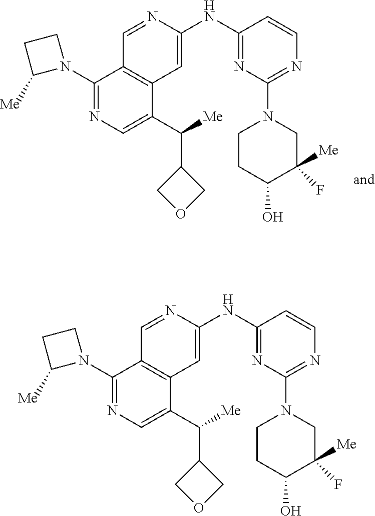 [SMILES: C[C@H](c1cnc(N2CC[C@H]2C)c2cnc(Nc3ccnc(N4CC[C@@H](O)[C@@](C)(F)C4)n3)cc12)C1COC1 ; C[C@@H]1CCN1c1ncc([C@H](C)C2COC2)c2cc(Nc3ccnc(N4CC[C@@H](O)[C@@](C)(F)C4)n3)ncc12]

Part 1. Into a 20-mL sealed tube was placed 6-chloro-1-((R)-2-methylazetidin-1-yl)-4-(1-(oxetan-3-yl)ethyl)-2,7-naphthyridine (Preparation 305, 45 mg, 0.141 mmol) in dioxane (5 mL), was added (3S,4R)-1-(4-aminopyrimidin-2-yl)-3-fluoro-3-methylpiperidin-4-ol (Preparation 8, 33.4 mg, 0.148 mmol), Cs₂CO₃ (20.4 mg, 0.063 mmol) and BretPhos Pd G3 (2.84 mg, 0.003 mmol) under N₂ and the resulting solution stirred at 120° C. for 3 h. The reaction mixture was diluted the H₂O (5 mL) and extracted with EtOAc (3×20 mL). The combined organics were washed with brine (10 mL), dried and evaporated to dryness in vacuo. The residue was purified by prep-HPLC-23 (Gradient (% organic) 33-45%) to afford (3S,4R)-3-fluoro-3-methyl-1-(4-((8-((R)-2-methylazetidin-1-yl)-5-(1-(oxetan-3-yl)ethyl)-2,7-naphthyridin-3-yl)amino)pyrimidin-2-yl)piperidin-4-ol as a yellow gum (20 mg, 28%).

Part 2. The compound of Part 1 was further purified by chiral-HPLC (Chiralpak IC-3, 4.6×50 mm, 3 mm; 50% EtOH/(3:1 Hex:DCM)(+0.1% DEA)) to afford the title compounds.

Peak 1 (Example 176) (3S,4R)-3-fluoro-3-methyl-1-(4-((8-((R)-2-methylazetidin-1-yl)-5-((S)-1-(oxetan-3-yl)ethyl)-2,7-naphthyridin-3-yl)amino)pyrimidin-2-yl)piperidin-4-ol or (3S,4R)-3-fluoro-3-methyl-1-(4-((8-((R)-2-methylazetidin-1-yl)-5-((R)-1-(oxetan-3-yl)ethyl)-2,7-naphthyridin-3-yl)amino)pyrimidin-2-yl)piperidin-4-ol

White solid, 8 mg; LCMS m/z=508 [M+H]*; ¹HNMR (300 MHz, DMSO-d₆) δ:10.06 (s, 1H), 9.03 (s, 1H), 8.42 (s, 1H), 8.05 (d, 1H), 7.81 (s, 1H), 6.56 (d, 1H), 5.04 (d, 1H), 4.84-4.63 (m, 6H), 4.67-4.55 (m, 1H), 4.19-4.09 (m, 1H), 4.06 (d, 1H), 3.60-3.51 (m, 3H), 3.14 (dd, 2H), 2.08 (q, 1H), 1.75 (d, 2H), 1.47-1.37 (m, 6H), 1.33 (s, 1H), 1.18 (d, 3H).

Peak 2 (Example 177) (3S,4R)-3-fluoro-3-methyl-1-(4-((8-((R)-2-methylazetidin-1-yl)-5-((S)-1-(oxetan-3-yl)ethyl)-2,7-naphthyridin-3-yl)amino)pyrimidin-2-yl)piperidin-4-ol or (3S,4R)-3-fluoro-3-methyl-1-(4-((8-((R)-2-methylazetidin-1-yl)-5-((R)-1-(oxetan-3-yl)ethyl)-2,7-naphthyridin-3-yl)amino)pyrimidin-2-yl)piperidin-4-ol. White solid, 12 mg; LCMS m/z=508 [M+H]⁺; ¹HNMR (300 MHz, DMSO-d₆) δ:10.06 (s, 1H), 9.04 (s, 1H), 8.45 (s, 1H), 8.05 (d, 1H), 7.82 (s, 1H), 6.56 (d, 1H), 5.03 (d, 1H), 4.87-4.65 (m, 6H), 4.70-4.47 (m, 1H), 4.22-4.11 (m, 1H), 4.10 (t, 1H), 3.68-3.52 (m, 3H), 3.19 (dd, 2H), 2.08 (q, 1H), 1.77 (d, 2H), 1.48-1.30 (m, 6H), 1.32 (s, 1H), 1.17 (d, 3H).

### Example 178

### (3S,4R)-3-fluoro-1-(4-((5-((S)-1-hydroxypropan-2-yl)-8-((R)-2-methylazetidin-1-yl)-2,6-naphthyridin-3-yl)amino)pyrimidin-2-yl)-3-methylpiperidin-4-ol or (3S,4R)-3-fluoro-1-(4-((5-((R)-1-hydroxypropan-2-yl)-8-((R)-2-methylazetidin-1-yl)-2,6-naphthyridin-3-yl)amino)pyrimidin-2-yl)-3-methylpiperidin-4-ol

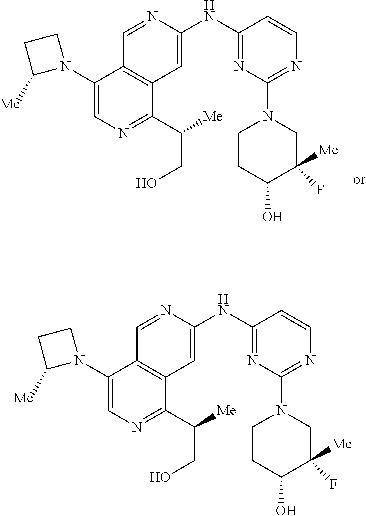 [SMILES: C[C@H](CO)c1ncc(N2CC[C@H]2C)c2cnc(Nc3ccnc(N4CC[C@@H](O)[C@@](C)(F)C4)n3)cc12 ; C[C@@H]1CCN1c1cnc([C@@H](C)CO)c2cc(Nc3ccnc(N4CC[C@@H](O)[C@@](C)(F)C4)n3)ncc12]

The title compound was prepared from 2-(7-chloro-4-((R)-2-methylazetidin-1-yl)-2,6-naphthyridin-1-yl)propan-1-ol (Preparation 349) and (3S,4R)-1-(4-aminopyrimidin-2-yl)-3-fluoro-3-methylpiperidin-4-ol (Preparation 8) using an analogous 2-part process to that described for Example 176 and 177. Chiralpak IE, 2×25 mm, 5 mm; 50% EtOH/Hex (+8 mM NH₃/MeOH).

Peak 2. Yellow solid, 43 mg; LCMS m/z=482 [M+H]⁺; ¹HNMR (400 MHz, DMSO-d₆) δ:10.06 (s, 1H), 9.12 (s, 1H), 8.72 (s, 1H), 8.02 (d, 1H), 7.72 (s, 1H), 6.44 (d, 1H), 5.02 (d, 1H), 4.83-4.68 (m, 2H), 4.63-4.46 (m, 3H), 3.90 (q, 1H), 3.76 (dd, 1H), 3.72-3.41 (m, 3H), 3.11 (m, 2H), 2.16-2.07 (m, 1H), 1.78-1.77 (m, 2H), 1.44-1.37 (m, 5H), 1.34 (s, 2H), 1.26 (d, 3H).

### Example 179

### (3S,4R)-3-fluoro-1-(5-((5-isopropyl-8-((R)-2-methylazetidin-1-yl)-2,7-naphthyridin-3-yl)amino)-1,2,4-triazin-3-yl)-3-methylpiperidin-4-ol or (3R,4S)-3-fluoro-1-(5-((5-isopropyl-8-((R)-2-methylazetidin-1-yl)-2,7-naphthyridin-3-yl)amino)-1,2,4-triazin-3-yl)-3-methylpiperidin-4-ol

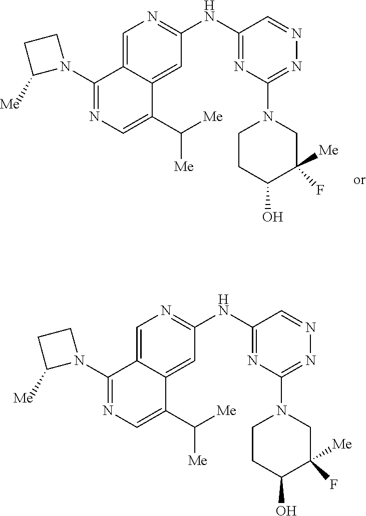 [SMILES: CC(C)c1cnc(N2CC[C@H]2C)c2cnc(Nc3cnnc(N4CC[C@@H](O)[C@@](C)(F)C4)n3)cc12 ; CC(C)c1cnc(N2CC[C@H]2C)c2cnc(Nc3cnnc(N4CC[C@H](O)[C@](C)(F)C4)n3)cc12]

A mixture of 1-(5-amino-1,2,4-triazin-3-yl)-3-fluoro-3-methylpiperidin-4-ol (Preparation 68, 86.3 mg, 0.380 mmol), (R)-6-chloro-4-isopropyl-1-(2-methylazetidin-1-yl)-2,7-naphthyridine (Preparation 191, 100 mg, 0.362 mmol), BrettPhos Pd G3 (65.6 mg, 0.074 mmol) and Cs₂CO₃ (236 mg, 0.724 mmol) in dioxane (5 mL) was heated at 100° C. under N₂ for 2 h. The reaction mixture was diluted with water and extracted into EtOAc. The combined extracts were dried (Na₂SO₄) and evaporated to dryness in vacuo. The residue was purified by prep-HPLC-23 (Gradient (% organic) 19-30%) and further purified by chiral-HPLC (Chiralpak ID-3, 4.6×50 mm, 3 mm; 7% EtOH/MTBE (+0.3% ⁱPrNH₂)) to afford the title compound.

Peak 2. White solid, 8 mg; LCMS m/z=467 [M+H]⁺; ¹HNMR (300 MHz, DMSO-d₆) δ:10.63 (s, 1H), 9.09 (s, 1H), 8.52 (d, 2H), 8.04 (s, 1H), 5.08 (d, 1H), 4.79 (q, 2H), 4.73-4.59 (m, 2H), 4.12 (q, 1H), 3.70-3.48 (m, 1H), 3.33 (s, 1H), 3.18 (d, 1H), 2.13-2.01 (m, 1H), 1.80-1.73 (m, 2H), 1.43 (d, 5H), 1.38-1.28 (m, 8H).

### Example 180

### (3S,4R)-3-fluoro-1-(4-((5-((R)-1-hydroxypropan-2-yl)-8-(3-methoxyazetidin-1-yl)isoquinolin-3-yl)amino)pyrimidin-2-yl)-3-methylpiperidin-4-ol or (3S,4R)-3-fluoro-1-(4-((5-((S)-1-hydroxypropan-2-yl)-8-(3-methoxyazetidin-1-yl)isoquinolin-3-yl)amino)pyrimidin-2-yl)-3-methylpiperidin-4-ol

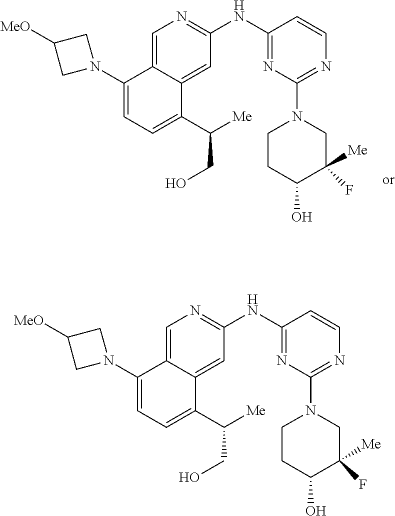 [SMILES: COC1CN(c2ccc([C@@H](C)CO)c3cc(Nc4ccnc(N5CC[C@@H](O)[C@@](C)(F)C5)n4)ncc23)C1 ; COC1CN(c2ccc([C@H](C)CO)c3cc(Nc4ccnc(N5CC[C@@H](O)[C@](C)(F)C5)n4)ncc23)C1]

The title compound was prepared from 2-(3-chloro-8-(3-methoxyazetidin-1-yl)isoquinolin-5-yl)propan-1-ol (Preparation 129) and (3S,4R)-1-(4-aminopyrimidin-2-yl)-3-fluoro-3-methylpiperidin-4-ol (Preparation 8) using an analogous 2-part process to that described for Example 176 and 177. Chiralpak IG, 20×250 mm, 5 mm; 50% EtOH/(Hex/DCM, 3:1 (+10 mM NH₃/MeOH)).

Peak 2. Yellow solid; LCMS m/z=497 [M+H]⁺; ¹HNMR (300 MHz, DMSO-d₆) δ: 9.83 (s, 1H), 9.05 (s, 1H), 8.59 (s, 1H), 7.99 (d, 1H), 7.38 (d, 1H), 6.46 (dd, 2H), 5.01 (d, 1H), 4.82-4.58 (m, 3H), 4.39 (d, 3H), 3.93 (d, 2H), 3.73-3.43 (m, 4H), 3.29 (s, 3H), 3.19-3.00 (m, 2H), 1.93-1.58 (m, 2H), 1.47-1.18 (m, 6H).

### Example 181

### (3S,4R)-1-(4-((5-ethyl-8-((S)-2-methylazetidin-1-yl)-2,7-naphthyridin-3-yl)amino)pyrimidin-2-yl)-3-fluoro-3-methylpiperidin-4-ol or (3S,4R)-1-(4-((5-ethyl-8-((R)-2-methylazetidin-1-yl)-2,7-naphthyridin-3-yl)amino)pyrimidin-2-yl)-3-fluoro-3-methylpiperidin-4-ol

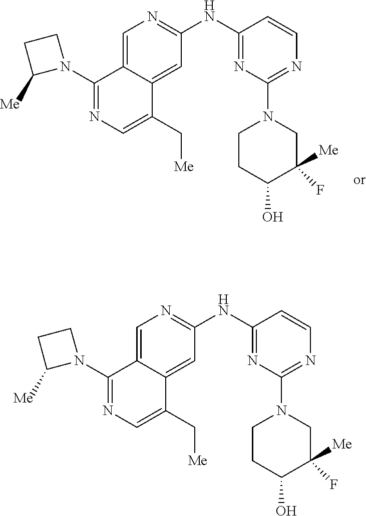 [SMILES: CCc1cnc(N2CC[C@@H]2C)c2cnc(Nc3ccnc(N4CC[C@@H](O)[C@@](C)(F)C4)n3)cc12 ; CCc1cnc(N2CC[C@H]2C)c2cnc(Nc3ccnc(N4CC[C@@H](O)[C@@](C)(F)C4)n3)cc12]

The title compound was prepared from 6-chloro-4-ethyl-1-(2-methylazetidin-1-yl)-2,7-naphthyridine (Preparation 296) and (3S,4R)-1-(4-aminopyrimidin-2-yl)-3-fluoro-3-methylpiperidin-4-ol (Preparation 8) using XPhos Pd G4 in an analogous 2-part process to that described for Example 176 and 177. Chiralpak IG, 20×250 mm, 5 mm; 50% EtOH/(Hex/DCM, 3:1 (+10 mM NH₃/MeOH)).

Peak 1. White solid; LCMS m/z=452 [M+H]⁺; ¹HNMR (300 MHz, DMSO-d₆) δ: 10.05 (s, 1H), 9.01 (s, 1H), 8.38 (s, 1H), 8.01 (d, 1H), 7.88 (s, 1H), 6.47 (d, 1H), 5.02 (d, 1H), 4.89-4.44 (m, 4H), 4.08 (q, 1H), 3.75-3.38 (m, 1H), 3.12 (q, 2H), 2.75 (q, 2H), 2.05 (q, 1H), 1.72 (s, 2H), 1.55-1.12 (m, 10H).

### Example 182

### (3S,4R)-1-(4-((5-ethoxy-8-((S)-2-methylazetidin-1-yl)-2,7-naphthyridin-3-yl)amino)pyrimidin-2-yl)-3-fluoro-3-methylpiperidin-4-ol or (3S,4R)-1-(4-((5-ethoxy-8-((R)-2-methylazetidin-1-yl)-2,7-naphthyridin-3-yl)amino)pyrimidin-2-yl)-3-fluoro-3-methylpiperidin-4-ol

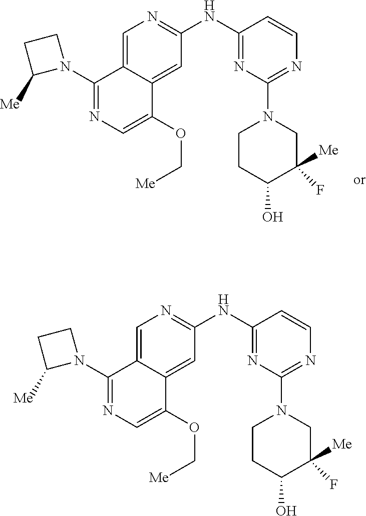 [SMILES: CCOc1cnc(N2CC[C@@H]2C)c2cnc(Nc3ccnc(N4CC[C@@H](O)[C@@](C)(F)C4)n3)cc12 ; CCOc1cnc(N2CC[C@H]2C)c2cnc(Nc3ccnc(N4CC[C@@H](O)[C@@](C)(F)C4)n3)cc12]

The title compound was prepared from 6-chloro-4-ethoxy-1-(2-methylazetidin-1-yl)-2,7-naphthyridine (Preparation 313) and (3S,4R)-1-(4-aminopyrimidin-2-yl)-3-fluoro-3-methylpiperidin-4-ol (Preparation 8) using XPhos Pd G4 in an analogous 2-part process to that described for Example 176 and 177. Chiralpak IG, 20×250 mm, 5 mm; 50% EtOH/(Hex/DCM, 3:1 (+10 mM NH₃/MeOH)).

Peak 2. White solid; LCMS m/z=468 [M+H]⁺; ¹HNMR (300 MHz, DMSO-d₆) δ: 10.09 (s, 1H), 9.00 (s, 1H), 8.53 (s, 1H), 8.01 (d, 1H), 7.73 (s, 1H), 6.44 (d, 1H), 4.98 (d, 1H), 4.86-4.41 (m, 5H), 4.14 (q, 2H), 3.96 (q, 1H), 3.46 (d, 1H), 3.24-2.86 (m, 1H), 2.04 (p, 1H), 1.69 (d, 2H), 1.50-1.21 (m, 10H).

### Example 183 and 184

### (3S,4R)-3-fluoro-3-methyl-1-(4-((8-((R)-2-methylazetidin-1-yl)-5-((R)-1-(oxetan-3-ylmethoxy)ethyl)-2,7-naphthyridin-3-yl)amino)pyrimidin-2-yl)piperidin-4-ol and (3S,4R)-3-fluoro-3-methyl-1-(4-((8-((R)-2-methylazetidin-1-yl)-5-((S)-1-(oxetan-3-ylmethoxy)ethyl)-2,7-naphthyridin-3-yl)amino)pyrimidin-2-yl)piperidin-4-ol

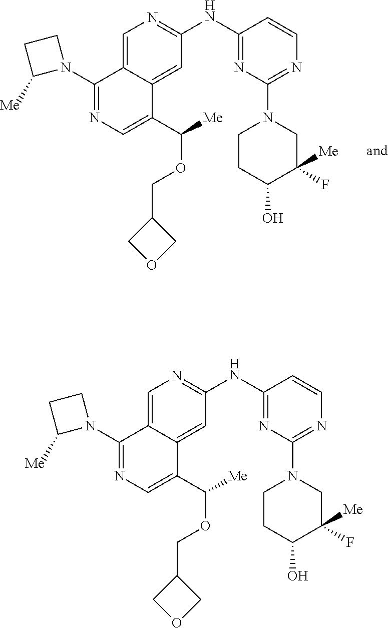 [SMILES: C[C@@H]1CCN1c1ncc([C@@H](C)OCC2COC2)c2cc(Nc3ccnc(N4CC[C@@H](O)[C@@](C)(F)C4)n3)ncc12 ; C[C@H](OCC1COC1)c1cnc(N2CC[C@H]2C)c2cnc(Nc3ccnc(N4CC[C@@H](O)[C@@](C)(F)C4)n3)cc12]

The title compound was prepared from 6-chloro-1-((R)-2-methylazetidin-1-yl)-4-(1-(oxetan-3-ylmethoxy)ethyl)-2,7-naphthyridine (Preparation 323) and (3S,4R)-1-(4-aminopyrimidin-2-yl)-3-fluoro-3-methylpiperidin-4-ol (Preparation 8) using XPhos Pd G4 in an analogous 2-part process to that described for Example 176 and 177. Chiralpak IE, 20×250 mm, 5 mm; 30% EtOH/(Hex/DCM, 3:1 (+10 mM NH₃/MeOH)).

Peak 1 (Example 183) (3S,4R)-3-fluoro-3-methyl-1-(4-((8-((R)-2-methylazetidin-1-yl)-5-((R)-1-(oxetan-3-ylmethoxy)ethyl)-2,7-naphthyridin-3-yl)amino)pyrimidin-2-yl)piperidin-4-ol or (3S,4R)-3-fluoro-3-methyl-1-(4-((8-((R)-2-methylazetidin-1-yl)-5-((S)-1-(oxetan-3-ylmethoxy)ethyl)-2,7-naphthyridin-3-yl)amino)pyrimidin-2-yl)piperidin-4-ol. Yellow solid (5.9 mg); LCMS m/z=538 [M+H]⁺; ¹HNMR (300 MHz, DMSO-d₆) δ: 10.00 (s, 1H), 9.02 (s, 1H), 8.52 (s, 1H), 8.05-7.93 (m, 2H), 6.54 (d, 1H), 4.99 (d, 1H), 4.85-4.75 (m, 2H), 4.78-4.60 (m, 3H), 4.51 (ddd, 2H), 4.15 (dt, 2H), 3.62-3.36 (m, 3H), 3.16-2.96 (m, 3H), 2.05 (s, 1H), 1.70 (s, 2H), 1.55-1.36 (m, 7H), 1.32 (s, 2H).

Peak 2 (Example 184) (3S,4R)-3-fluoro-3-methyl-1-(4-((8-((R)-2-methylazetidin-1-yl)-5-((R)-1-(oxetan-3-ylmethoxy)ethyl)-2,7-naphthyridin-3-yl)amino)pyrimidin-2-yl)piperidin-4-ol or (3S,4R)-3-fluoro-3-methyl-1-(4-((8-((R)-2-methylazetidin-1-yl)-5-((S)-1-(oxetan-3-ylmethoxy)ethyl)-2,7-naphthyridin-3-yl)amino)pyrimidin-2-yl)piperidin-4-ol. Yellow solid (10.3 mg); LCMS m/z=538 [M+H]⁺; ¹HNMR (300 MHz, DMSO-d₆) δ: 10.00 (s, 1H), 9.02 (s, 1H), 8.52 (s, 1H), 8.05-7.93 (m, 2H), 6.54 (d, 1H), 4.99 (d, 1H), 4.85-4.75 (m, 2H), 4.78-4.60 (m, 3H), 4.51 (ddd, 2H), 4.15 (dt, 2H), 3.62-3.36 (m, 3H), 3.16-2.96 (m, 3H), 2.05 (s, 1H), 1.70 (s, 2H), 1.55-1.36 (m, 7H), 1.32 (s, 2H).

### Example 185

### (R)-2-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)-N,N-dimethylpropanamide or (S)-2-(6-((2-((3S,4R)-3-fluoro-4-hydroxy-3-methylpiperidin-1-yl)pyrimidin-4-yl)amino)-1-((R)-2-methylazetidin-1-yl)-2,7-naphthyridin-4-yl)-N,N-dimethylpropanamide

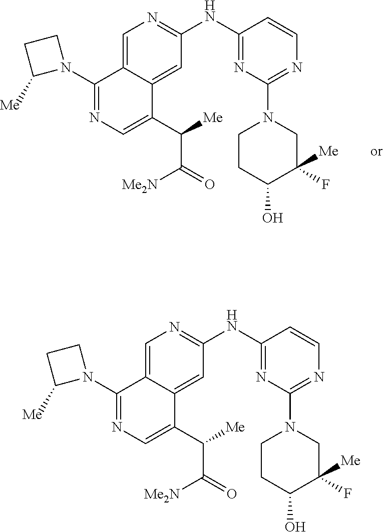 [SMILES: C[C@@H]1CCN1c1ncc([C@@H](C)C(=O)N(C)C)c2cc(Nc3ccnc(N4CC[C@@H](O)[C@@](C)(F)C4)n3)ncc12 ; C[C@H](C(=O)N(C)C)c1cnc(N2CC[C@H]2C)c2cnc(Nc3ccnc(N4CC[C@@H](O)[C@@](C)(F)C4)n3)cc12]

The title compound was prepared from 2-(6-chloro-1-((R)-2-methylazetidin-1-yl)-2,7-naphthyridin-4-yl)-N,N-dimethylpropanamide (Preparation 327) and (3S,4R)-1-(4-aminopyrimidin-2-yl)-3-fluoro-3-methylpiperidin-4-ol (Preparation 8) using XPhos Pd G4 in an analogous 2-part process to that described for Example 176 and 177. Chiralpak ID-03, 20×250 mm, 5 mm; 20% EtOH/(MTBE+10 mM NH₃/MeOH)).

Peak 2. Yellow solid (30.9 mg); LCMS m/z=523 [M+H]¹; ¹HNMR (400 MHz, DMSO-d₆) δ: 10.10 (s, 1H), 9.05 (s, 1H), 8.38 (s, 1H), 8.05 (d, 1H), 7.69 (s, 1H), 6.58 (d, 1H), 5.03 (d, 1H), 4.78 (dt, 1H), 4.76-4.62 (m, 3H), 4.25-4.16 (m, 1H), 4.16 (d, 1H), 3.54 (dq, 1H), 3.22-3.06 (m, 2H), 2.89 (s, 3H), 2.71 (s, 3H), 2.58-2.50 (m, 1H), 2.12-2.00 (m, 1H), 1.68 (q2H), 1.44 (d, 3H), 1.38-1.28 (m, 6H).

### Example 186

### (3S,4R)-3-fluoro-1-(4-((5-((R)-3-hydroxy-3-methylbutan-2-yl)-8-((R)-2-methylazetidin-1-yl)-2,7-naphthyridin-3-yl)amino)pyrimidin-2-yl)-3-methylpiperidin-4-ol or (3S,4R)-3-fluoro-1-(4-((5-((S)-3-hydroxy-3-methylbutan-2-yl)-8-((R)-2-methylazetidin-1-yl)-2,7-naphthyridin-3-yl)amino)pyrimidin-2-yl)-3-methylpiperidin-4-ol

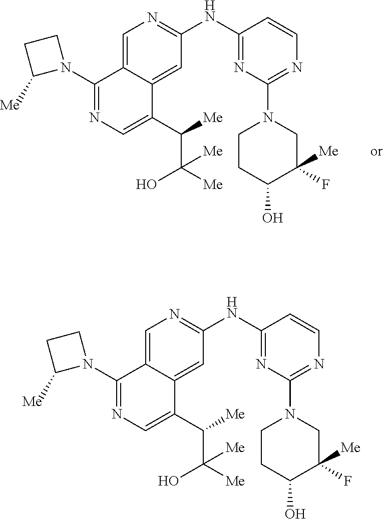 [SMILES: C[C@@H]1CCN1c1ncc([C@@H](C)C(C)(C)O)c2cc(Nc3ccnc(N4CC[C@@H](O)[C@@](C)(F)C4)n3)ncc12 ; C[C@@H]1CCN1c1ncc([C@H](C)C(C)(C)O)c2cc(Nc3ccnc(N4CC[C@@H](O)[C@@](C)(F)C4)n3)ncc12]

The title compound was prepared from 3-(6-chloro-1-((R)-2-methylazetidin-1-yl)-2,7-naphthyridin-4-yl)-2-methylbutan-2-ol (Preparation 326) and (3S,4R)-1-(4-aminopyrimidin-2-yl)-3-fluoro-3-methylpiperidin-4-ol (Preparation 8) using XPhos Pd G4 in an analogous 2-part process to that described for Example 176 and 177. Chiralpak IC, 20×250 mm, 5 mm; 30% EtOH/(Hex+8 mM NH₃/MeOH)).

Peak 2. Yellow solid; LCMS m/z=510 [M+H]⁺; ¹HNMR (400 MHz, DMSO-d₆) δ: 10.14 (s, 1H), 9.05 (s, 1H), 8.39 (s, 1H), 8.06 (d, 1H), 7.90 (s, 1H), 6.68 (s, 1H), 5.02 (d, 1H), 4.93 (s, 1H), 4.80 (s, 1H), 4.72 (dd, 1H), 4.65 (d, 1H), 4.46 (s, 1H), 4.32 (s, 1H), 3.52 (s, 1H), 3.14 (dt, 2H), 2.62 (s, 1H), 2.14-2.04 (m, 1H), 1.74 (s, 2H), 1.68 (d, 1H), 1.50 (d, 3H), 1.37 (s, 2H), 1.34-1.27 (m, 4H), 1.17 (s, 3H), 1.03 (s, 3H).

### Example 187 and 188

### (4S,5R)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol and (4S,5R)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4R,5S)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol and (4R,5S)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol

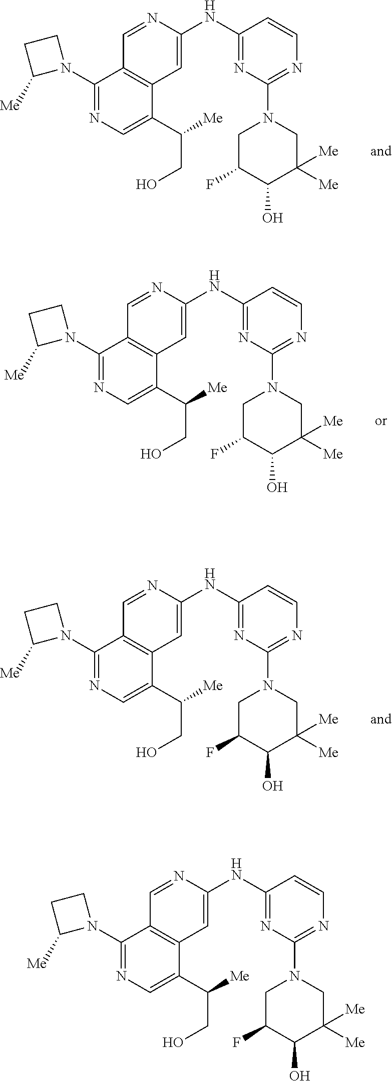 [SMILES: C[C@H](CO)c1cnc(N2CC[C@H]2C)c2cnc(Nc3ccnc(N4C[C@@H](F)[C@@H](O)C(C)(C)C4)n3)cc12 ; C[C@@H]1CCN1c1ncc([C@@H](C)CO)c2cc(Nc3ccnc(N4C[C@@H](F)[C@@H](O)C(C)(C)C4)n3)ncc12 ; C[C@H](CO)c1cnc(N2CC[C@H]2C)c2cnc(Nc3ccnc(N4C[C@H](F)[C@H](O)C(C)(C)C4)n3)cc12 ; C[C@@H]1CCN1c1ncc([C@@H](C)CO)c2cc(Nc3ccnc(N4C[C@H](F)[C@H](O)C(C)(C)C4)n3)ncc12]

The title compound was prepared from 2-(6-chloro-1-((R)-2-methylazetidin-1-yl)-2,7-naphthyridin-4-yl)propan-1-ol (Preparation 298) and (4S,5R)-1-(4-aminopyrimidin-2-yl)-5-fluoro-3,3-dimethylpiperidin-4-ol or (4R,5S)-1-(4-aminopyrimidin-2-yl)-5-fluoro-3,3-dimethylpiperidin-4-ol (Peak 2, Preparation 21 and 22) using XPhos Pd G4 in an analogous 2-part process to that described for Example 176 and 177. Chiralpak IE, 20×250 mm, 5 mm; 5% EtOH/(MTBE+10 mM NH₃/MeOH)).

Peak 1 (Example 187) (4S,5R)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4S,5R)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4R,5S)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4R,5S)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol. White solid (12.3 mg); LCMS m/z=496 [M+H]⁺; ¹HNMR (300 MHz, DMSO-d₆) δ: 9.99 (s, 1H), 9.00 (s, 1H), 8.43 (s, 1H), 8.01 (d, 1H), 7.93 (s, 1H), 6.51 (d, 1H), 5.10 (d, 1H), 4.90-4.52 (m, 4H), 4.45-4.22 (m, 1H), 4.08 (q, 1H), 3.84 (t, 2H), 3.68-3.36 (m, 5H), 2.04 (h, 1H), 1.42 (d, 3H), 1.25 (d, 3H), 0.91 (d, 6H).

Peak 2 (Example 188) (4S,5R)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4S,5R)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4R,5S)-5-fluoro-1-(4-((5-((S)-1-hydroxypropan-2-yl)-8-((R)-2-methylazetidin-1-yl)-2,7-naphthyridin-3-yl)amino)pyrimidin-2-yl)-3,3-dimethylpiperidin-4-ol or (4R,5S)-5-fluoro-1-(4-((5-((R)-1-hydroxypropan-2-yl)-8-((R)-2-methylazetidin-1-yl)-2,7-naphthyridin-3-yl)amino)pyrimidin-2-yl)-3,3-dimethylpiperidin-4-ol. White solid (10.8 mg); LCMS m/z=496 [M+H]*; ¹HNMR (300 MHz, DMSO-d₆) δ: 9.98 (s, 1H), 9.00 (s, 1H), 8.38 (s, 1H), 8.01 (d, 1H), 7.94 (s, 1H), 6.51 (d, 1H), 5.10 (d, 1H), 4.87-4.50 (m, 4H), 4.31 (q, 1H), 4.08 (q, 1H), 3.95-3.73 (m, 2H), 3.72-3.45 (m, 4H), 2.05 (t, 1H), 1.41 (d, 3H), 1.26 (d, 3H), 0.90 (d, 6H).

### Example 189

### (3S,4R)-3-fluoro-1-(4-((5-((R)-1-hydroxypropan-2-yl)-8-((S)-2-methylazetidin-1-yl)-2,7-naphthyridin-3-yl)amino)pyrimidin-2-yl)-3-methylpiperidin-4-ol or (3S,4R)-3-fluoro-1-(4-((5-((S)-1-hydroxypropan-2-yl)-8-((S)-2-methylazetidin-1-yl)-2,7-naphthyridin-3-yl)amino)pyrimidin-2-yl)-3-methylpiperidin-4-ol or (3S,4R)-3-fluoro-1-(4-((5-((R)-1-hydroxypropan-2-yl)-8-((R)-2-methylazetidin-1-yl)-2,7-naphthyridin-3-yl)amino)pyrimidin-2-yl)-3-methylpiperidin-4-ol or (3S,4R)-3-fluoro-1-(4-((5-((S)-1-hydroxypropan-2-yl)-8-((R)-2-methylazetidin-1-yl)-2,7-naphthyridin-3-yl)amino)pyrimidin-2-yl)-3-methylpiperidin-4-ol

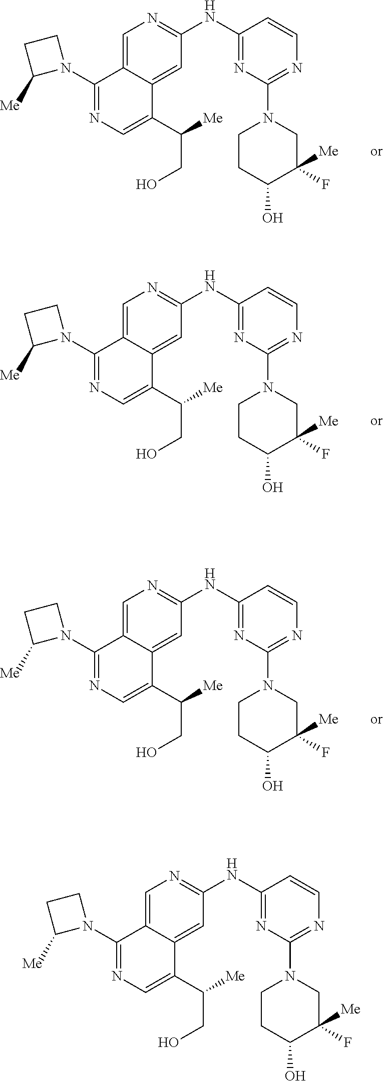 [SMILES: C[C@@H](CO)c1cnc(N2CC[C@@H]2C)c2cnc(Nc3ccnc(N4CC[C@@H](O)[C@@](C)(F)C4)n3)cc12 ; C[C@H](CO)c1cnc(N2CC[C@@H]2C)c2cnc(Nc3ccnc(N4CC[C@@H](O)[C@@](C)(F)C4)n3)cc12 ; C[C@@H]1CCN1c1ncc([C@@H](C)CO)c2cc(Nc3ccnc(N4CC[C@@H](O)[C@@](C)(F)C4)n3)ncc12 ; C[C@H](CO)c1cnc(N2CC[C@H]2C)c2cnc(Nc3ccnc(N4CC[C@@H](O)[C@@](C)(F)C4)n3)cc12]

Part 1. A mixture of (S)-2-(6-chloro-1-((R)-2-methylazetidin-1-yl)-2,7-naphthyridin-4-yl)propan-1-ol and (R)-2-(6-chloro-1-((S)-2-methylazetidin-1-yl)-2,7-naphthyridin-4-yl)propan-1-ol or (R)-2-(6-chloro-1-((R)-2-methylazetidin-1-yl)-2,7-naphthyridin-4-yl)propan-1-ol and (S)-2-(6-chloro-1-((S)-2-methylazetidin-1-yl)-2,7-naphthyridin-4-yl)propan-1-ol (Peak 1, Preparation 371, 34 mg, 0.117 mmol), (3S,4R)-1-(4-aminopyrimidin-2-yl)-3-fluoro-3-methylpiperidin-4-ol (Preparation 8, 26 mg, 0.117 mmol), Cs₂CO₃ (76 mg, 0.233 mmol) and XPhos Pd G4 (5 mg, 5.83 mmol) in dioxane (1 mL) was heated under N₂ for 1 h at 90° C. The mixture was diluted with 5% MeOH/DCM and washed with H₂O. The combined organics were dried (Na₂SO₄) and evaporated to dryness in vacuo and the residue purified by ISCO (0-10% MeOH/DCM) to afford a mixture of diastereomers as an amorphous solid.

Part 2. The compound of Part 1 was purified by chiral-HPLC (Chiralpak IC, 20×250 mm, 5 mm; 50% EtOH/(Hex/DCM, 3:1 (+10 mM NH₃/MeOH)).

Peak 2. White solid; LCMS m/z=482 [M+H]⁺; ¹HNMR (400 MHz, MeOH-d₄) δ: 9.06 (d, 1H), 8.49 (s, 1H), 8.12-7.98 (m, 1H), 7.91 (d, 1H), 6.45 (d, 1H), 4.76-4.66 (m, 3H), 4.20 (d, 1H), 3.91-3.60 (m, 3H), 3.47 (p, 1H), 3.30-3.10 (m, 2H), 2.66 (s, 1H), 2.14 (s, 1H), 2.05-1.83 (m, 2H), 1.61-1.34 (m, 10H).

### Example 190

### (3S,4R)-3-fluoro-1-(4-((5-isopropyl-8-(1,6-diazaspiro[3.3]heptan-1-yl)-2,7-naphthyridin-3-yl)amino)pyrimidin-2-yl)-3-methylpiperidin-4-ol

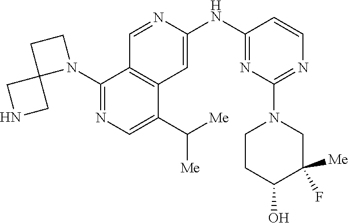 [SMILES: CC(C)c1cnc(N2CCC23CNC3)c2cnc(Nc3ccnc(N4CC[C@@H](O)[C@@](C)(F)C4)n3)cc12]

Part 1: tert-butyl 1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.3]heptane-6-carboxylate was prepared from tert-butyl 1-(6-chloro-4-isopropyl-2,7-naphthyridin-1-yl)-1,6-diazaspiro[3.3]heptane-6-carboxylate (Preparation 158) and (3S,4R)-1-(4-aminopyrimidin-2-yl)-3-fluoro-3-methylpiperidin-4-ol (Preparation 8) using an analogous method to that described for Example 29. Prep-TLC (20:1 DCM/MeOH); Yellow solid (200 mg) which was used without further purification in Part 2; LCMS m/z=593 [M+H]⁺

Part 2: The compound of Part 1 was dissolved in DCM (1.5 mL) and TFA (0.5 mL) added dropwise and the mixture stirred at rt for 2 h. The reaction mixture was evaporated to dryness in vacuo and the residue purified by prep-HPLC-3 (Gradient (% organic): 5-48%) to afford the title compound as a yellow solid (30 mg). LCMS m/z=493 [M+H]⁺; ¹HNMR (300 MHz, MeOH-d₄) δ: 9.30 (s, 1H), 8.33 (s, 1H), 8.24 (s, 1H), 7.93 (d, 1H), 4.96-4.84 (m, 2H), 4.68 (t, 2H), 4.41 (d, 3H), 3.88-3.39 (m, 5H), 2.77 (t, 2H), 2.12-1.88 (m, 2H), 1.66-1.37 (m, 9H).

### Example 191

### (R)—N-(2-(5,5-difluoro-1-oxa-7-azaspiro[3.5]nonan-7-yl)pyrimidin-4-yl)-5-isopropyl-8-(1,6-diazaspiro[3.3]heptan-1-yl)-2,7-naphthyridin-3-amine or (S)—N-(2-(5,5-difluoro-1-oxa-7-azaspiro[3.5]nonan-7-yl)pyrimidin-4-yl)-5-isopropyl-8-(1,6-diazaspiro[3.3]heptan-1-yl)-2,7-naphthyridin-3-amine

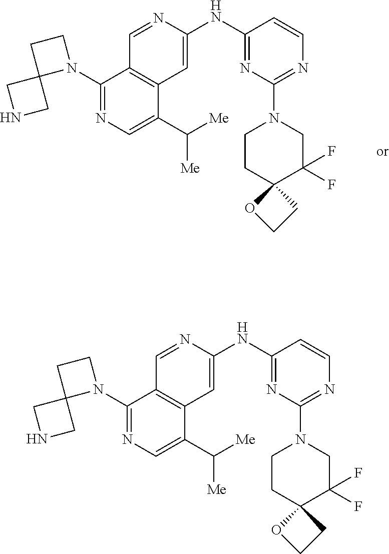 [SMILES: CC(C)c1cnc(N2CCC23CNC3)c2cnc(Nc3ccnc(N4CC[C@@]5(CCO5)C(F)(F)C4)n3)cc12 ; CC(C)c1cnc(N2CCC23CNC3)c2cnc(Nc3ccnc(N4CC[C@]5(CCO5)C(F)(F)C4)n3)cc12]

The title compound was prepared from using an analogous 2-part process to that described for Example 190 using tert-butyl 1-(6-chloro-4-isopropyl-2,7-naphthyridin-1-yl)-1,6-diazaspiro[3.3]heptane-6-carboxylate (Preparation 158) and (S)-2-(5,5-difluoro-1-oxa-7-azaspiro[3.5]nonan-7-yl)pyrimidin-4-amine or (R)-2-(5,5-difluoro-1-oxa-7-azaspiro[3.5]nonan-7-yl)pyrimidin-4-amine (Peak 2, Preparation 66 and 67). Prep-HPLC-4 Gradient (% organic): 20-30%) to afford title compound as white solid (3 mg). LCMS m/z=523 [M+H]⁺; ¹HNMR (400 MHz, MeOH-d₄) δ: 9.22 (s, 1H), 8.49 (s, 1H), 8.18 (s, 1H), 8.04 (d, 1H), 6.61 (d, 1H), 4.70-4.51 (m, 5H), 4.41 (d, 2H), 4.28 (d, 1H), 3.86 (dd, 1H), 3.59 (t, 1H), 3.50-3.40 (m, 1H), 3.00-2.86 (m, 1H), 2.75 (t, 2H), 2.57 (q, 1H), 2.30 (d, 1H), 2.18-2.01 (m, 1H), 1.44 (dd, 6H).

### Example 192

### (3S,4R)-3-fluoro-1-(4-((5-isopropyl-8-(6-methyl-1,6-diazaspiro[3.3]heptan-1-yl)-2,7-naphthyridin-3-yl)amino)pyrimidin-2-yl)-3-methylpiperidin-4-ol trifluoroacetate

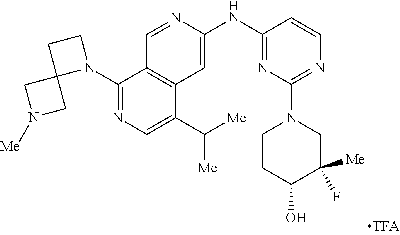 [SMILES: CC(C)c1cnc(N2CCC23CN(C)C3)c2cnc(Nc3ccnc(N4CC[C@@H](O)[C@@](C)(F)C4)n3)cc12]

A mixture of (3S,4R)-3-fluoro-1-(4-((5-isopropyl-8-(1,6-diazaspiro[3.3]heptan-1-yl)-2,7-naphthyridin-3-yl)amino)pyrimidin-2-yl)-3-methylpiperidin-4-ol (Example 190, 200 mg, 0.406 mmol), HCHO (121 mg, 4.06 mmol) and NaBH₃CN (255 mg, 4.06 mmol) in MeOH was stirred at rt for 2 hr. The reaction mixture was evaporated to dryness and the residue partitioned between EtOAc H₂O. The combined organics were dried (Na₂SO₄) and evaporated to dryness in vacuo. The residue was purified by prep-HPLC-19 (Gradient (% organic) 25-50%) to afford the title compound as a yellow solid (30 mg, 14%). LCMS m/z=507 [M+H]⁺; ¹HNMR (300 MHz, MeOH-d₄) δ: 9.28 (s, 1H), 8.35 (s, 1H), 8.24 (s, 1H), 7.93 (d, 1H), 6.95 (s, 1H), 5.44-4.92 (m, 2H), 4.66 (t, 3H), 4.53-4.22 (m, 3H), 3.83-3.33 (m, 4H), 3.02 (s, 3H), 2.76 (t, 2H), 2.03-1.88 (m, 2H), 1.62-1.35 (m, 9H).

### Example 193

### (3S,4R)-3-fluoro-1-(4-((5-isopropyl-8-(6-isopropyl-1,6-diazaspiro[3.3]heptan-1-yl)-2,7-naphthyridin-3-yl)amino)pyrimidin-2-yl)-3-methylpiperidin-4-ol

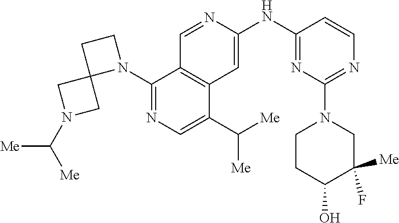 [SMILES: CC(C)c1cnc(N2CCC23CN(C(C)C)C3)c2cnc(Nc3ccnc(N4CC[C@@H](O)[C@@](C)(F)C4)n3)cc12]

The title compound was prepared from (3S,4R)-3-fluoro-1-(4-((5-isopropyl-8-(1,6-diazaspiro[3.3]heptan-1-yl)-2,7-naphthyridin-3-yl)amino)pyrimidin-2-yl)-3-methylpiperidin-4-ol (Example 190, 100 mg, 0.203 mmol) and acetone using an analogous method to that described for Example 192. The residue was purified by prep-HPLC-18 (Gradient (% organic) 50-75%) to afford the title compound as a yellow solid (7 mg, 6%). LCMS m/z=535 [M+H]⁺; ¹HNMR (300 MHz, DMSO-d₆) δ: 10.00 (s, 1H), 9.12 (s, 1H), 8.40 (s, 1H), 8.01 (d, 1H), 7.93 (s, 1H), 6.49 (d, 1H), 5.03 (d), 4.78-4.40 (m, 4H), 4.24 (d, 2H), 3.67-3.44 (m, 1H), 3.27-3.01 (m, 5H), 2.74-2.55 (m, 3H), 1.83-1.60 (m, 2H), 1.46-1.23 (m, 9H), 0.88 (d, 6H).

### Example 194

### (R)—N-(2-(5,5-difluoro-1-oxa-7-azaspiro[3.5]nonan-7-yl)pyrimidin-4-yl)-5-isopropyl-8-(6-methyl-1,6-diazaspiro[3.3]heptan-1-yl)-2,7-naphthyridin-3-amine or (S)—N-(2-(5,5-difluoro-1-oxa-7-azaspiro[3.5]nonan-7-yl)pyrimidin-4-yl)-5-isopropyl-8-(6-methyl-1,6-diazaspiro[3.3]heptan-1-yl)-2,7-naphthyridin-3-amine

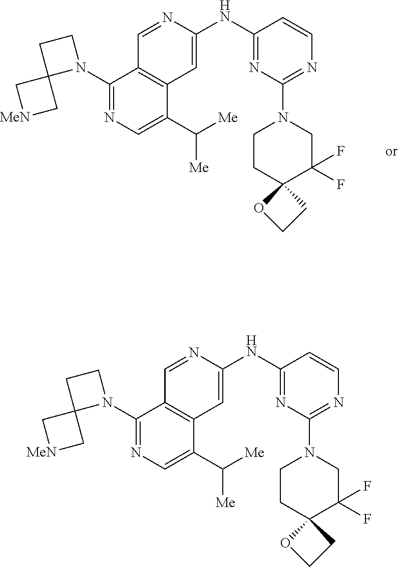 [SMILES: CC(C)c1cnc(N2CCC23CN(C)C3)c2cnc(Nc3ccnc(N4CC[C@@]5(CCO5)C(F)(F)C4)n3)cc12 ; CC(C)c1cnc(N2CCC23CN(C)C3)c2cnc(Nc3ccnc(N4CC[C@]5(CCO5)C(F)(F)C4)n3)cc12]

The title compound was prepared from Example 191 using an analogous method to that described for Example 192. Prep-HPLC-27 (Gradient (% organic) 18-35%) to afford the title compound as a white solid (28.7 mg). LCMS m/z=537 [M+H]⁺; ¹HNMR (300 MHz, DMSO-d₆) δ: 10.16 (s, 1H), 9.16 (s, 1H), 8.39 (s, 1H), 8.08 (d, 1H), 8.03 (s, 1H), 6.62 (d, 1H), 4.69-4.22 (m, 7H), 4.13-3.56 (m, 7H), 2.88-2.72 (m, 1H), 2.66-2.56 (m, 5H), 2.17-1.93 (m, 2H), 1.35 (d, 6H).

### Example 195

### (3S,4R)-3-fluoro-1-(4-((5-isopropyl-8-(6-methyl-1,6-diazaspiro[3.3]heptan-1-yl)-2,6-naphthyridin-3-yl)amino)pyrimidin-2-yl)-3-methylpiperidin-4-ol

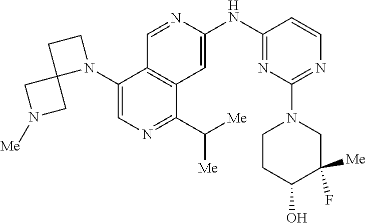 [SMILES: CC(C)c1ncc(N2CCC23CN(C)C3)c2cnc(Nc3ccnc(N4CC[C@@H](O)[C@@](C)(F)C4)n3)cc12]

Part 1. Part 1 tert-butyl 1-(7-((2-((3S,4R)-3-fluoro-4-hydroxy-3-methylpiperidin-1-yl)pyrimidin-4-yl)amino)-1-isopropyl-2,6-naphthyridin-4-yl)-1,6-diazaspiro[3.3]heptane-6-carboxylate was prepared from tert-butyl 1-(7-chloro-1-isopropyl-2,6-naphthyridin-4-yl)-1,6-diazaspiro[3.3]heptane-6-carboxylate (Preparation 347) and (3S,4R)-1-(4-aminopyrimidin-2-yl)-3-fluoro-3-methylpiperidin-4-ol (Preparation 8) using an analogous method to that described for Example 29. Prep-TLC (10:1 DCM/MeOH); Yellow solid (75 mg) which was used without further purification in Part 2; LCMS m/z=593 [M+H]⁺

Part 2. The compound of Part 1 was dissolved in DCM (2 mL) and TFA (1 mL) added dropwise and the mixture stirred at rt for 2 h. The reaction mixture was evaporated to dryness in vacuo and the residue diluted with water and the pH adjusted to ˜6-7 with aq. NaHCO₃ and extracted with DCM (2×20 mL). The combined organics were dried (Na₂SO₄) and evaporated to dryness in vacuo to afford (3S,4R)-3-fluoro-1-(4-((5-isopropyl-8-(1,6-diazaspiro[3.3]heptan-1-yl)-2,6-naphthyridin-3-yl)amino)pyrimidin-2-yl)-3-methylpiperidin-4-ol as a yellow solid (50 mg). LCMS m/z=493 [M+H]⁺

Part 3. The title compound was prepared from the compound of Part 2 and HCHO using and analogous method to that described for Example 192. The residue was purified by prep-HPLC-10 (Gradient (% organic): 24-39%) to afford the title compound as a yellow solid (7 mg). LCMS m/z=507 [M+H]⁺; ¹HNMR (300 MHz, DMSO-d₆) δ: 10.04 (s, 1H), 9.31 (s, 1H), 8.66 (s, 1H), 8.61 (s, 1H), 8.00 (d, 1H), 6.42 (d, 1H), 5.04 (d, 1H), 4.82-4.54 (m, 2H), 4.27 (t, 2H), 3.90 (d, 2H), 3.79-3.46 (m, 3H), 3.25-2.98 (m, 5H), 2.26 (s, 3H), 1.84-1.57 (m, 2H), 1.48-1.22 (m, 9H).

### Example 196

### (3S,4R)-3-fluoro-1-(4-((5-isopropyl-8-(7-methyl-1,7-diazaspiro[3.5]nonan-1-yl)-2,7-naphthyridin-3-yl)amino)pyrimidin-2-yl)-3-methylpiperidin-4-ol

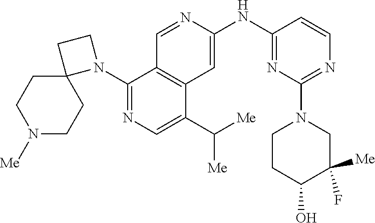 [SMILES: CC(C)c1cnc(N2CCC23CCN(C)CC3)c2cnc(Nc3ccnc(N4CC[C@@H](O)[C@@](C)(F)C4)n3)cc12]

The title compound was prepared from tert-butyl 1-(6-chloro-4-isopropyl-2,7-naphthyridin-1-yl)-1,7-diazaspiro[3.5]nonane-7-carboxylate (Preparation 188) and (3S,4R)-1-(4-aminopyrimidin-2-yl)-3-fluoro-3-methylpiperidin-4-ol (Preparation 8) using an analogous 3-part process as described for Example 195. LCMS m/z=535 [M+H]⁺; ¹HNMR (300 MHz, DMSO-d₆) δ: 9.95 (s, 1H), 9.11 (s, 1H), 8.38 (s, 1H), 8.02 (d, 1H), 7.88 (s, 1H), 6.51 (d, 1H), 5.02 (d, 1H), 4.77-4.62 (m, 2H), 4.56 (t, 2H), 3.69-3.43 (m, 1H), 3.29-3.05 (m, 3H), 2.92 (t, 2H), 2.78-2.61 (m, 3H), 2.37-2.26 (m, 1H), 2.20 (t, 2H), 2.15 (s, 3H), 1.89 (t, 2H), 1.74-1.61 (m, 4H), 1.40-1.24 (m, 9H).

### Example 197

### (3S,4R)-3-fluoro-1-(4-((5-isopropyl-8-((S)-2-((methylamino)methyl)pyrrolidin-1-yl)-2,7-naphthyridin-3-yl)amino)pyrimidin-2-yl)-3-methylpiperidin-4-ol

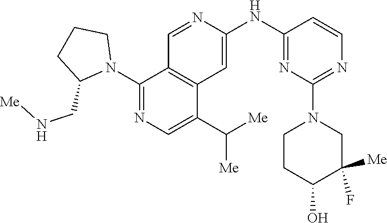 [SMILES: CNC[C@@H]1CCCN1c1ncc(C(C)C)c2cc(Nc3ccnc(N4CC[C@@H](O)[C@@](C)(F)C4)n3)ncc12]

The title compound was prepared from tert-butyl (S)-((1-(6-chloro-4-isopropyl-2,7-naphthyridin-1-yl)pyrrolidin-2-yl)methyl)(methyl)carbamate (Preparation 232) and (3S,4R)-1-(4-aminopyrimidin-2-yl)-3-fluoro-3-methylpiperidin-4-ol (Preparation 8) using an analogous 2-part process as described for Example 190. Prep-HPLC-27 (% organic=11%). LCMS m/z=509 [M+H]⁺; ¹HNMR (300 MHz, DMSO-d₆) δ: 10.10 (s, 1H), 9.23 (s, 1H), 8.46 (s, 1H), 8.34 (s, 1H), 8.01 (d, 1H), 7.97 (s, 1H), 6.47 (d, 1H), 4.73 (d, 3H), 4.12-3.99 (m, 1H), 3.74-3.44 (m, 2H), 3.42-3.27 (m, 1H), 3.21-2.89 (m, 4H), 2.45 (s, 3H), 2.11 (s, 1H), 1.90 (d, 2H), 1.72 (s, 3H), 1.47-1.07 (m, 9H).

### Example 198

### (3S,4R)-3-fluoro-1-(4-((5-isopropyl-8-((S)-6-methyl-1,6-diazaspiro[3.4]octan-1-yl)-2,7-naphthyridin-3-yl)amino)pyrimidin-2-yl)-3-methylpiperidin-4-ol or (3S,4R)-3-fluoro-1-(4-((5-isopropyl-8-((R)-6-methyl-1,6-diazaspiro[3.4]octan-1-yl)-2,7-naphthyridin-3-yl)amino)pyrimidin-2-yl)-3-methylpiperidin-4-ol

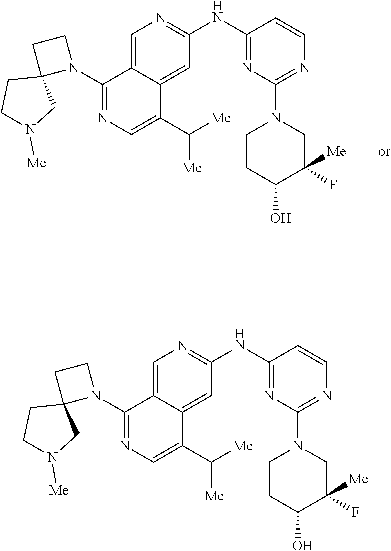 [SMILES: CC(C)c1cnc(N2CC[C@]23CCN(C)C3)c2cnc(Nc3ccnc(N4CC[C@@H](O)[C@@](C)(F)C4)n3)cc12 ; CC(C)c1cnc(N2CC[C@@]23CCN(C)C3)c2cnc(Nc3ccnc(N4CC[C@@H](O)[C@@](C)(F)C4)n3)cc12]

Part 1. To a solution of tert-butyl (S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate or tert-butyl (R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate (Peak 1 from Preparation 330 and 331, 70 mg, 0.115 mmol) in DCM (3 mL) was added TFA (1 mL) and the mixture stirred for 1 h at rt. The reaction mixture was evaporated to dryness in vacuo to afford (3S,4R)-3-fluoro-1-(4-((5-isopropyl-8-((R)-1,6-diazaspiro[3.4]octan-1-yl)-2,7-naphthyridin-3-yl)amino)pyrimidin-2-yl)-3-methylpiperidin-4-ol or (3S,4R)-3-fluoro-1-(4-((5-isopropyl-8-((S)-1,6-diazaspiro[3.4]octan-1-yl)-2,7-naphthyridin-3-yl)amino)pyrimidin-2-yl)-3-methylpiperidin-4-ol as a colourless oil. LCMS m/z=507 [M+H]⁺

Part 2. NaBH₃CN (20.8 mg, 0.47 mmol) was added to a solution of the compound of Part 1 (80 mg, 0.156 mmol) and AcOH (36.5 mg, 0.609 mmol) in MeOH and the resulting solution stirred at rt for 2 h. The reaction mixture was quenched with H₂O (50 mL) and extracted with EtOAc (2×50 mL). The combined organics were dried (Na₂SO₄) and evaporated to dryness in vacuo. The residue was purified by prep-HPLC-27 (Gradient (% organic) 2-28%) to afford the title compound as an off-white solid (17 mg, 20%). LCMS m/z=521 [M+H]⁺; ¹HNMR (300 MHz, DMSO-d₆) δ: 10.05 (s, 1H), 9.18 (s, 1H), 8.43 (s, 1H), 8.24 (s, 0.5H), 8.03 (d, 1H), 7.96 (s, 1H), 6.51 (d, 1H), 4.72 (dd, 2H), 4.53 (q, 2H), 3.60 (s, 4H), 3.14 (d, 4H), 2.95-2.66 (m, 2H), 2.52 (s, 1H), 2.47 (s, 3H), 2.04 (s, 1H), 1.73 (s, 2H), 1.48-1.21 (m, 9H).

### Example 199

### (3S,4R)-3-fluoro-1-(4-((5-isopropyl-8-((R)-6-methyl-1,6-diazaspiro[3.4]octan-1-yl)-2,7-naphthyridin-3-yl)amino)pyrimidin-2-yl)-3-methylpiperidin-4-ol or (3S,4R)-3-fluoro-1-(4-((5-isopropyl-8-((S)-6-methyl-1,6-diazaspiro[3.4]octan-1-yl)-2,7-naphthyridin-3-yl)amino)pyrimidin-2-yl)-3-methylpiperidin-4-ol

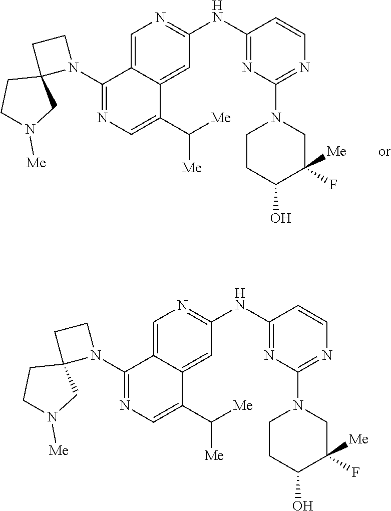 [SMILES: CC(C)c1cnc(N2CC[C@@]23CCN(C)C3)c2cnc(Nc3ccnc(N4CC[C@@H](O)[C@@](C)(F)C4)n3)cc12 ; CC(C)c1cnc(N2CC[C@]23CCN(C)C3)c2cnc(Nc3ccnc(N4CC[C@@H](O)[C@@](C)(F)C4)n3)cc12]

The title compound was prepared from tert-butyl (R)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate or tert-butyl (S)-1-(6-((2-((3S,4R)-3-fluoro-4-hydroxy-3-methylpiperidin-1-yl)pyrimidin-4-yl)amino)-4-isopropyl-2,7-naphthyridin-1-yl)-1,6-diazaspiro[3.4]octane-6-carboxylate (Peak 2 from Preparation 330 and 331) using an analogous 2-part process as described for Example 198. prep-HPLC-27 (Gradient (% organic) 2-28%). LCMS m/z=521 [M+H]⁺; ¹HNMR (300 MHz, DMSO-d₆) δ: 10.05 (s, 1H), 9.18 (s, 1H), 8.43 (s, 1H), 8.25 (s, 0.5H), 8.03 (d, 1H), 7.96 (s, 1H), 6.51 (d, 1H), 4.86-4.60 (m, 2H), 4.53 (q, 2H), 3.60 (t, 1H), 3.37-3.20 (m, 2H), 3.12 (t, 4H), 2.93-2.66 (m, 2H), 2.52 (d, 1H), 2.46 (s, 4H), 2.03 (s, 1H), 1.73 (s, 2H), 1.50-1.18 (m, 9H).

### Example 200

### N-(2-((3R,4S)-3-fluoro-4-(2-(methylamino)ethoxy)piperidin-1-yl)pyrimidin-4-yl)-5-isopropyl-8-((R)-2-methylazetidin-1-yl)-2,7-naphthyridin-3-amine

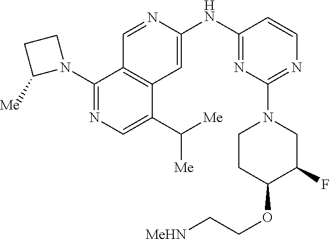 [SMILES: CNCCO[C@H]1CCN(c2nccc(Nc3cc4c(C(C)C)cnc(N5CC[C@H]5C)c4cn3)n2)C[C@H]1F]

Part 1. MsCl (34.7 mg, 0.303 mmol) was added to a solution of 2-(((3R,4S)-3-fluoro-1-(4-((5-isopropyl-8-((R)-2-methylazetidin-1-yl)-2,7-naphthyridin-3-yl)amino)pyrimidin-2-yl)piperidin-4-yl)oxy)ethan-1-ol (Example 141, 100 mg, 0.202 mmol) and TEA (61.3 mg, 0.605 mmol) in DCM (10 mL) and stirred at 0° C. for 1 h. The solution was evaporated to dryness in vacuo to afford 2-(((3R,4S)-3-fluoro-1-(4-((5-isopropyl-8-((R)-2-methylazetidin-1-yl)-2,7-naphthyridin-3-yl)amino)pyrimidin-2-yl)piperidin-4-yl)oxy)ethyl methanesulfonate as a pale yellow solid (130 mg) which was used without further purification. LCMS m/z=574 [M+H]⁺

Part 2. A mixture of the compound of Part 1 (50 mg, 0.087 mmol) and methylamine (39.3 mg, 0.873 mmol) in MeOH (10 mL) was heated at 70° C. for 1 h. The reaction mixture was evaporated to dryness in vacuo and the residue purified by prep-HPLC-21 (Gradient (% organic) 36-41%) to afford the title compound was a pale yellow solid (10 mg, 22%). LCMS m/z=509 [M+H]⁺; ¹HNMR (400 MHz, DMSO-d₆) δ: 10.07 (s, 1H), 9.03 (s, 1H), 8.48 (s, 1H), 8.04 (d, 1H), 7.98 (s, 1H), 6.52 (d, 1H), 4.97 (s, 1H), 4.78 (q, 1H), 4.70 (s, 1H), 4.63 (q, 1H), 4.46 (d, 1H), 4.10 (q, 1H), 3.60 (t, 3H), 3.48 (dd, 4H), 2.64 (t, 3H), 2.31 (s, 3H), 2.06 (t, 1H), 1.86-1.71 (m, 2H), 1.43 (d, 3H), 1.32 (dd, 6H).

### Example 201

### N-(2-((3S,4R)-3-fluoro-4-(2-(methylamino)ethoxy)piperidin-1-yl)pyrimidin-4-yl)-5-isopropyl-8-((R)-2-methylazetidin-1-yl)-2,7-naphthyridin-3-amine

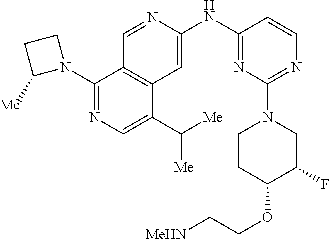 [SMILES: CNCCO[C@@H]1CCN(c2nccc(Nc3cc4c(C(C)C)cnc(N5CC[C@H]5C)c4cn3)n2)C[C@@H]1F]

The title compound was prepared from 2-(((3S,4R)-3-fluoro-1-(4-((5-isopropyl-8-((R)-2-methylazetidin-1-yl)-2,7-naphthyridin-3-yl)amino)pyrimidin-2-yl)piperidin-4-yl)oxy)ethan-1-ol (Example 142) and methylamine using an analogous 2-part process as described for Example 200. Yellow solid (10 mg, 22.5%). LCMS m/z=509 [M+H]*; ¹HNMR (400 MHz, DMSO-d₆) δ: 10.08 (s, 1H), 9.03 (s, 1H), 8.49 (s, 1H), 8.04 (d, 1H), 7.98 (s, 1H), 6.51 (d, 1H), 4.86 (d, 1H), 4.77 (dt, 2H), 4.74-4.58 (m, 1H), 4.46 (d, 1H), 4.10 (q, 1H), 3.61 (q, 2H), 3.32 (s, 6H), 2.67 (t, 2H), 2.32 (s, 3H), 2.13-2.00 (m, 1H), 1.85-1.71 (m, 2H), 1.43 (d, 3H), 1.32 (dd, 6H).

### Example 202

### N-(2-((3R,4S)-4-(2-(dimethylamino)ethoxy)-3-fluoropiperidin-1-yl)pyrimidin-4-yl)-5-isopropyl-8-((R)-2-methylazetidin-1-yl)-2,7-naphthyridin-3-amine

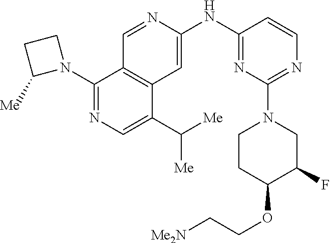 [SMILES: CC(C)c1cnc(N2CC[C@H]2C)c2cnc(Nc3ccnc(N4CC[C@H](OCCN(C)C)[C@H](F)C4)n3)cc12]

The title compound was prepared from 2-(((3R,4S)-3-fluoro-1-(4-((5-isopropyl-8-((R)-2-methylazetidin-1-yl)-2,7-naphthyridin-3-yl)amino)pyrimidin-2-yl)piperidin-4-yl)oxy)ethan-1-ol (Example 141) and dimethylamine using an analogous 2-part process as described for Example 200. Prep-HPLC-18 (Gradient (% organic) 44-54%) afforded title compound as pale yellow solid (10 mg, 22%). LCMS m/z=523 [M+H]⁺; ¹HNMR (300 MHz, DMSO-d₆) δ: 10.07 (s, 1H), 9.03 (s, 1H), 8.48 (s, 1H), 8.07-8.01 (m, 1H), 7.98 (s, 1H), 6.52 (d, 1H), 4.98 (d, 1H), 4.86 (d, 1H), 4.79 (p, 1H), 4.71-4.58 (m, 1H), 4.45 (d, 1H), 4.10 (q, 1H), 3.78-3.57 (m, 3H), 3.48 (dd, 1H), 2.46 (t, 5H), 2.19 (d, 6H), 2.06 (t, 1H), 1.84-1.71 (m, 2H), 1.43 (d, 3H), 1.32 (dd, 6H).

### Example 203

### N-(2-((3S,4R)-4-(2-(dimethylamino)ethoxy)-3-fluoropiperidin-1-yl)pyrimidin-4-yl)-5-isopropyl-8-((R)-2-methylazetidin-1-yl)-2,7-naphthyridin-3-amine

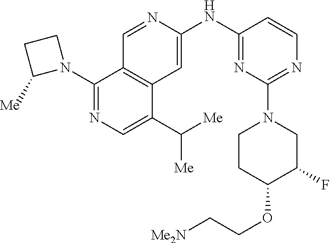 [SMILES: CC(C)c1cnc(N2CC[C@H]2C)c2cnc(Nc3ccnc(N4CC[C@@H](OCCN(C)C)[C@@H](F)C4)n3)cc12]

The title compound was prepared from 2-(((3S,4R)-3-fluoro-1-(4-((5-isopropyl-8-((R)-2-methylazetidin-1-yl)-2,7-naphthyridin-3-yl)amino)pyrimidin-2-yl)piperidin-4-yl)oxy)ethan-1-ol (Example 142) and methylamine using an analogous 2-part process as described for Example 200. Yellow solid (10 mg, 22.6%). LCMS m/z=523 [M+H]⁺; ¹HNMR (300 MHz, DMSO-d₆) δ:10.08 (s, 1H), 9.03 (s, 1H), 8.49 (s, 1H), 8.04 (d, 1H), 7.98 (s, 1H), 6.51 (d, 1H), 4.84-4.58 (m, 4H), 4.45 (d, 1H), 4.10 (q, 1H), 3.74 (d, 3H), 3.44 (tt, 1H), 3.28 (dd, 2H), 2.45 (d, 3H), 2.18 (dd, 6H), 2.13-2.00 (m, 1H), 1.86-1.71 (m, 2H), 1.43 (d, 3H), 1.31 (dd, 6H).

### Example 204

### (3S,4R)-1-(4-((8-((S)-2-(difluoromethyl)azetidin-1-yl)-5-isopropyl-2,7-naphthyridin-3-yl)amino)pyrimidin-2-yl)-3-fluoro-3-methylpiperidin-4-ol or (3S,4R)-1-(4-((8-((R)-2-(difluoromethyl)azetidin-1-yl)-5-isopropyl-2,7-naphthyridin-3-yl)amino)pyrimidin-2-yl)-3-fluoro-3-methylpiperidin-4-ol

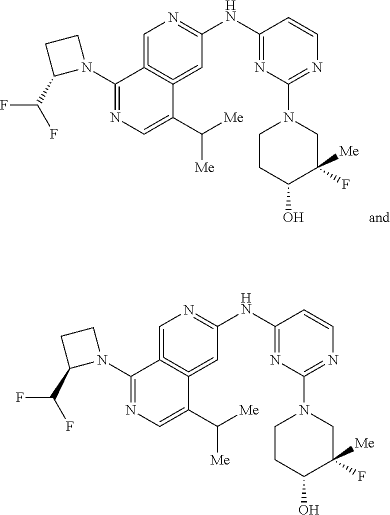

Part 1: (3S,4R)-1-(4-((8-(2-(difluoromethyl)azetidin-1-yl)-5-isopropyl-2,7-naphthyridin-3-yl)amino)pyrimidin-2-yl)-3-fluoro-3-methylpiperidin-4-ol was prepared from 6-chloro-1-(2-(difluoromethyl)azetidin-1-yl)-4-isopropyl-2,7-naphthyridine (Preparation 180) and (3S,4R)-1-(4-aminopyrimidin-2-yl)-3-fluoro-3-methylpiperidin-4-ol (Preparation 8) using an analogous method to that described for Example 44 (BRETTPHOS). Prep-HPLC-23 (Gradient (% organic): 45-56%). Yield: 800 mg, 41% as pale yellow solid.

Part 2: The compound of Part 1 was purified by chiral HPLC (CHIRALPAK IG-3; 4.6×50 mm, 3 mm; 50% Hex (+0.1% DEA)/EtOH to afford the title compounds as white solids

Peak 1 (Example 204) (3S,4R)-1-(4-((8-((S)-2-(difluoromethyl)azetidin-1-yl)-5-isopropyl-2,7-naphthyridin-3-yl)amino)pyrimidin-2-yl)-3-fluoro-3-methylpiperidin-4-ol or (3S,4R)-1-(4-((8-((R)-2-(difluoromethyl)azetidin-1-yl)-5-isopropyl-2,7-naphthyridin-3-yl)amino)pyrimidin-2-yl)-3-fluoro-3-methylpiperidin-4-ol: White solid (300 mg); LCMS m/z=502 [M+H]⁺; ¹HNMR (400 MHz, DMSO-d₆) δ: 10.11 (s, 1H), 9.08 (s, 1H), 8.53 (s, 1H), 8.15-7.94 (m, 2H), 6.62-6.18 (m, 2H), 5.13-4.95 (m, 2H), 4.72 (ddd, 3H), 4.25 (dt, 1H), 3.57 (dt, 1H), 3.38 (d, 1H), 3.23-3.07 (m, 2H), 2.56 (td, 1H), 2.49-2.39 (m, 1H), 1.76 (d, 2H), 1.43-1.29 (m, 9H).

Peak 2: White solid (285 mg); LCMS m/z=502 [M+H]⁺; ¹HNMR (400 MHz, DMSO-d₆) δ: 10.10 (s, 1H), 9.08 (s, 1H), 8.53 (s, 1H), 8.21-7.91 (m, 2H), 6.62-6.18 (m, 2H), 5.01 (dd, 2H), 4.86-4.60 (m, 2H), 4.26 (q, 1H), 3.66-3.49 (m, 1H), 3.38 (d, 1H), 3.15 (q, 2H), 2.66-2.54 (m, 1H), 2.44 (dp, 1H), 1.75 (d, 2H), 1.49-1.28 (m, 9H).

### Example 205

### (3S,4R)-1-(4-((4-((S)-2-(difluoromethyl)azetidin-1-yl)-1-isopropylpyrido[3,4-d]pyridazin-7-yl)amino)pyrimidin-2-yl)-3-fluoro-3-methylpiperidin-4-ol or (3S,4R)-1-(4-((4-((R)-2-(difluoromethyl)azetidin-1-yl)-1-isopropylpyrido[3,4-d]pyridazin-7-yl)amino)pyrimidin-2-yl)-3-fluoro-3-methylpiperidin-4-ol

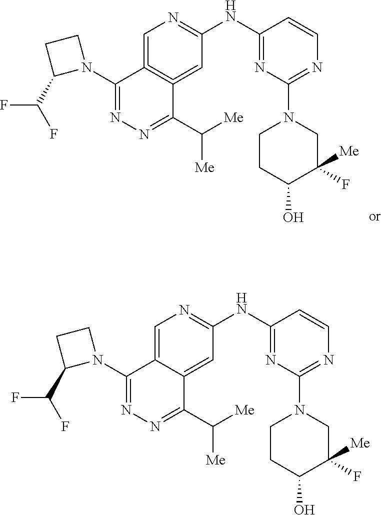

The title compounds were prepared from 7-chloro-4-(2-(difluoromethyl)azetidin-1-yl)-1-isopropylpyrido[3,4-d]pyridazine (Preparation 366) and (3S,4R)-1-(4-aminopyrimidin-2-yl)-3-fluoro-3-methylpiperidin-4-ol (Preparation 8) using an analogous 2-part procedure as described for Example 204. Chiral-HPLC ((R,R)-WHELK-01-Kromasil, 50×250 mm, 5 mm); 5% EtOH/MTBE (10 mM NH₃/MEOH)) afforded:

Peak 1: Pale yellow solid (21 mg); LCMS m/z=503 [M+H]⁺; ¹HNMR (400 MHz, DMSO-d₆) δ: 10.47 (s, 1H), 9.14 (s, 1H), 8.61 (s, 1H), 8.07 (d, 1H), 6.53-6.46 (m, 2H), 5.05-5.35 (m, 2H), 4.75-4.72 (m, 3H), 4.47 (s, 1H), 3.61-3.58 (m, 2H), 3.31-3.10 (m, 3H), 2.50-2.49 (m, 1H), 1.73 (s, 2H), 1.40-1.33 (m, 9H).

### Example 206

### (3S,4R)-3-fluoro-1-(4-((1-isopropyl-4-((S)-2-methylazetidin-1-yl)pyrido[3,4-d]pyridazin-7-yl)amino)pyrimidin-2-yl)-3-methylpiperidin-4-ol or (3S,4R)-3-fluoro-1-(4-((1-isopropyl-4-((S)-2-methylazetidin-1-yl)pyrido[3,4-d]pyridazin-7-yl)amino)pyrimidin-2-yl)-3-methylpiperidin-4-ol

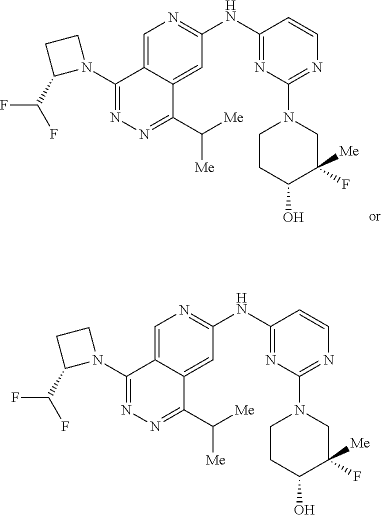 [SMILES: CC(C)c1nnc(N2CC[C@H]2C(F)F)c2cnc(Nc3ccnc(N4CC[C@@H](O)[C@@](C)(F)C4)n3)cc12 ; "or" ; CC(C)c1nnc(N2CC[C@H]2C(F)F)c2cnc(Nc3ccnc(N4CC[C@@H](O)[C@@](C)(F)C4)n3)cc12]

The title compound was prepared from 7-chloro-1-isopropyl-4-(2-methylazetidin-1-yl)pyrido[3,4-d]pyridazine (Preparation 367) and (3S,4R)-1-(4-aminopyrimidin-2-yl)-3-fluoro-3-methylpiperidin-4-ol (Preparation 8) using a similar 2-part process as described for Example 204. Purification by prep-HPLC ((R,R)-WHELK-01-Kromasil, 50×250 mm, 5 mm); 5% EtOH/MTBE (10 mM NH₃·MeOH)) afforded the title compound:

Peak 2, pale yellow solid (19 mg). LCMS m/z=467 [M+H]⁺; ¹HNMR (400 MHz, DMSO-d₆) δ: 10.36 (s, 1H), 9.09 (s, 1H), 8.57 (s, 1H), 8.08 (s, 1H), 6.51 (d, 1H), 5.08 (d, 1H), 4.88 (q, 1H), 4.80-4.52 (m, 3H), 4.18 (q, 1H), 3.69-3.46 (m, 3H), 3.25-3.00 (m, 2H), 2.24-1.96 (m, 1H), 1.87-1.68 (m, 2H), 1.55-1.29 (m, 12H).

### Example 207 and 208

### (3S,4R)-3-fluoro-1-(4-((5-isopropyl-8-((R)-3-((methylsulfonyl)methyl)pyrrolidin-1-yl)-2,7-naphthyridin-3-yl)amino)pyrimidin-2-yl)-3-methylpiperidin-4-ol and (3S,4R)-3-fluoro-1-(4-((5-isopropyl-8-((S)-3-((methylsulfonyl)methyl)pyrrolidin-1-yl)-2,7-naphthyridin-3-yl)amino)pyrimidin-2-yl)-3-methylpiperidin-4-ol

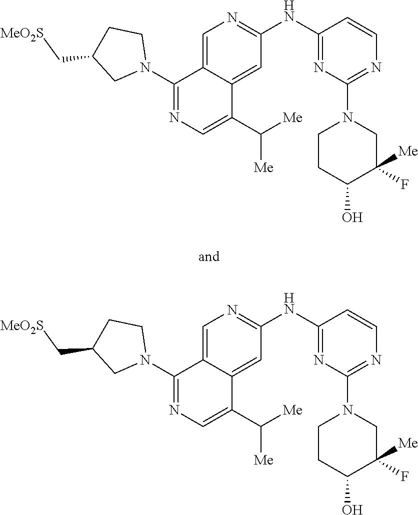 [SMILES: CC(C)c1cnc(N2CC[C@@H](CS(C)(=O)=O)C2)c2cnc(Nc3ccnc(N4CC[C@@H](O)[C@@](C)(F)C4)n3)cc12 ; "and" ; CC(C)c1cnc(N2CC[C@H](CS(C)(=O)=O)C2)c2cnc(Nc3ccnc(N4CC[C@@H](O)[C@@](C)(F)C4)n3)cc12]

The title compounds were prepared from 6-chloro-4-isopropyl-1-(3-((methylsulfonyl)methyl)pyrrolidin-1-yl)-2,7-naphthyridine (Preparation 210) and (3S,4R)-1-(4-aminopyrimidin-2-yl)-3-fluoro-3-methylpiperidin-4-ol (Preparation 8) using an analogous 2-part procedure as described for Example 204. Chiral-HPLC (Chiralpak IC-3, 4.6×50 mm, 3 mm; 50% EtOH/(Hex/DCM, 3:1+0.1% DEA)) afforded:

Peak 1 (Example 207) (3S,4R)-3-fluoro-1-(4-((5-isopropyl-8-((R)-3-((methylsulfonyl)methyl)pyrrolidin-1-yl)-2,7-naphthyridin-3-yl)amino)pyrimidin-2-yl)-3-methylpiperidin-4-ol or (3S,4R)-3-fluoro-1-(4-((5-isopropyl-8-((S)-3-((methylsulfonyl)methyl)pyrrolidin-1-yl)-2,7-naphthyridin-3-yl)amino)pyrimidin-2-yl)-3-methylpiperidin-4-ol: White solid; LCMS m/z=558 [M+H]⁺; ¹HNMR (300 MHz, DMSO-d₆) δ: 10.02 (s, 1H), 9.26 (s, 1H), 8.42 (s, 1H), 8.01 (d, 1H), 7.94 (s, 1H), 6.49 (d, 1H), 5.02 (d, 1H), 4.79-4.60 (m, 2H), 3.94 (dd, 3H), 3.87 (s, 1H), 3.80 (q, 1H), 3.73-3.55 (m, 1H), 3.55-3.33 (m, 2H), 3.23-3.08 (m, 2H), 3.02 (s, 3H), 2.75 (d, 1H), 2.24 (s, 1H), 1.72 (s, 3H), 1.42-1.19 (m, 9H).

Peak 2 (Example 208) (3S,4R)-3-fluoro-1-(4-((5-isopropyl-8-((R)-3-((methylsulfonyl)methyl)pyrrolidin-1-yl)-2,7-naphthyridin-3-yl)amino)pyrimidin-2-yl)-3-methylpiperidin-4-ol or (3S,4R)-3-fluoro-1-(4-((5-isopropyl-8-((S)-3-((methylsulfonyl)methyl)pyrrolidin-1-yl)-2,7-naphthyridin-3-yl)amino)pyrimidin-2-yl)-3-methylpiperidin-4-ol: White solid; LCMS m/z=558 [M+H]⁺; ¹HNMR (300 MHz, DMSO-d₆) δ: 10.03 (s, 1H), 9.25 (s, 1H), 8.42 (s, 1H), 8.01 (d, 1H), 7.95 (s, 1H), 6.49 (d, 1H), 5.02 (d, 1H), 4.79-4.60 (m, 2H), 3.92 (q, 3H), 3.88-3.75 (m, 1H), 3.74-3.54 (m, 1H), 3.54-3.34 (m, 3H), 3.23-3.04 (m, 2H), 3.02 (s, 3H), 2.22 (s, 1H), 1.80 (t, 1H), 1.78 (m, 3H), 1.38-1.30 (m, 9H).

### Example 209 and 210

### (3S,4R)-3-fluoro-1-(4-((8-((2R,3R)-3-fluoro-2-methylazetidin-1-yl)-5-((R)-1-hydroxypropan-2-yl)-2,7-naphthyridin-3-yl)amino)pyrimidin-2-yl)-3-methylpiperidin-4-ol and (3S,4R)-3-fluoro-1-(4-((8-((2R,3R)-3-fluoro-2-methylazetidin-1-yl)-5-((S)-1-hydroxypropan-2-yl)-2,7-naphthyridin-3-yl)amino)pyrimidin-2-yl)-3-methylpiperidin-4-ol

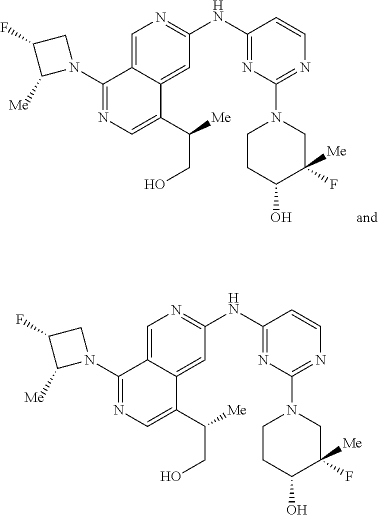 [SMILES: C[C@@H]1[C@H](F)CN1c1ncc([C@@H](C)CO)c2cc(Nc3ccnc(N4CC[C@@H](O)[C@@](C)(F)C4)n3)ncc12 ; "and" ; C[C@H](CO)c1cnc(N2C[C@@H](F)[C@H]2C)c2cnc(Nc3ccnc(N4CC[C@@H](O)[C@@](C)(F)C4)n3)cc12]

The title compounds were prepared from 2-(6-chloro-1-((2R,3R)-3-fluoro-2-methylazetidin-1-yl)-2,7-naphthyridin-4-yl)propan-1-ol (Preparation 299) and (3S,4R)-1-(4-aminopyrimidin-2-yl)-3-fluoro-3-methylpiperidin-4-ol (Preparation 8) using an analogous 2-part procedure as described for Example 204 using RuPhos Pd G3 as catalyst. Chiral-HPLC (Chiralpak IA-3, 4.6×50 mm, 3 mm); 30% EtOH/Hex (+0.1% DEA)) afforded:

Peak 1 (Example 209) (3S,4R)-3-fluoro-1-(4-((8-((2R,3R)-3-fluoro-2-methylazetidin-1-yl)-5-((R)-1-hydroxypropan-2-yl)-2,7-naphthyridin-3-yl)amino)pyrimidin-2-yl)-3-methylpiperidin-4-ol or (3S,4R)-3-fluoro-1-(4-((8-((2R,3R)-3-fluoro-2-methylazetidin-1-yl)-5-((S)-1-hydroxypropan-2-yl)-2,7-naphthyridin-3-yl)amino)pyrimidin-2-yl)-3-methylpiperidin-4-ol: Off-white solid (2.2 mg); LCMS m/z=500 [M+H]⁺; ¹HNMR (300 MHz, MeOH-d₄) δ: 9.09 (s, 1H), 8.55 (s, 1H), 8.05-7.96 (m, 2H), 6.47 (d, 1H), 5.21 (dt, 1H), 5.07-4.94 (m, 1H), 4.74 (dd, 2H), 4.13 (ddd, 1H), 3.87 (dd, 1H), 3.82-3.59 (m, 2H), 3.50 (q, 1H), 3.32-3.14 (m, 1H), 1.94 (q, 2H), 1.57-1.37 (m, 8H).

Peak 2 (Example 210) (3S,4R)-3-fluoro-1-(4-((8-((2R,3R)-3-fluoro-2-methylazetidin-1-yl)-5-((R)-1-hydroxypropan-2-yl)-2,7-naphthyridin-3-yl)amino)pyrimidin-2-yl)-3-methylpiperidin-4-ol or (3S,4R)-3-fluoro-1-(4-((8-((2R,3R)-3-fluoro-2-methylazetidin-1-yl)-5-((S)-1-hydroxypropan-2-yl)-2,7-naphthyridin-3-yl)amino)pyrimidin-2-yl)-3-methylpiperidin-4-ol: Off-white solid (2.1 mg); LCMS m/z=500 [M+H]⁺; ¹HNMR (300 MHz, MeOH-d₄) δ: 9.09 (s, 1H), 8.55 (s, 1H), 8.05-7.96 (m, 2H), 6.47 (d, 1H), 5.21 (dt, 1H), 5.07-4.94 (m, 1H), 4.74 (dd, 2H), 4.13 (ddd, 1H), 3.87 (dd, 1H), 3.82-3.59 (m, 2H), 3.50 (q, 1H), 3.32-3.14 (m, 1H), 1.94 (q, 2H), 1.57-1.37 (m, 8H)

### Example 211 and 212

### (3S,4R)-3-fluoro-1-(4-((5-((R)-1-hydroxypropan-2-yl)-8-((2R,3S)-3-methoxy-2-methylazetidin-1-1)-2,7-naphthyridin-3-yl)amino)pyrimidin-2-yl)-3-methylpiperidin-4-ol and (3S,4R)-3-fluoro-1-(4-((5-((S)-1-hydroxypropan-2-yl)-8-((2R,3S)-3-methoxy-2-methylazetidin-1-yl)-2,7-naphthyridin-3-yl)amino)pyrimidin-2-yl)-3-methylpiperidin-4-ol

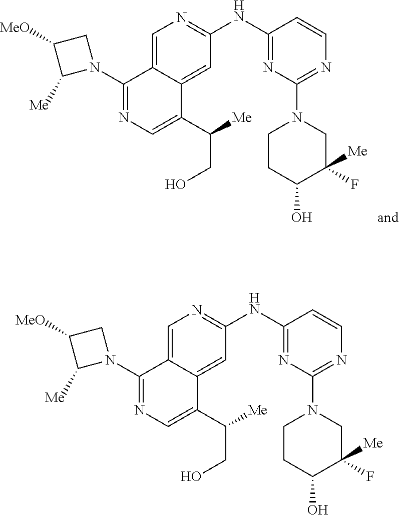 [SMILES: CO[C@@H]1CN(c2ncc([C@@H](C)CO)c3cc(Nc4ccnc(N5CC[C@@H](O)[C@@](C)(F)C5)n4)ncc23)[C@@H]1C ; "and" ; CO[C@@H]1CN(c2ncc([C@H](C)CO)c3cc(Nc4ccnc(N5CC[C@@H](O)[C@@](C)(F)C5)n4)ncc23)[C@@H]1C]

The title compounds were prepared from 2-(6-chloro-1-((2R,3S)-3-methoxy-2-methylazetidin-1-yl)-2,7-naphthyridin-4-yl)propan-1-ol (Preparation 297) and (3S,4R)-1-(4-aminopyrimidin-2-yl)-3-fluoro-3-methylpiperidin-4-ol (Preparation 8) using an analogous 2-part procedure as described for Example 204 using BrettPhos Pd G4 as catalyst. Chiral-HPLC (Chiralpak IA-3, 20×250 mm, 5 mm); 50% EtOH/(3:1 Hex/DCM+10 mM NH₃/MeOH) afforded:

Peak 1 (Example 211) (3S,4R)-3-fluoro-1-(4-((5-((R)-1-hydroxypropan-2-yl)-8-((2R,3S)-3-methoxy-2-methylazetidin-1-yl)-2,7-naphthyridin-3-yl)amino)pyrimidin-2-yl)-3-methylpiperidin-4-ol or (3S,4R)-3-fluoro-1-(4-((5-((S)-1-hydroxypropan-2-yl)-8-((2R,3S)-3-methoxy-2-methylazetidin-1-yl)-2,7-naphthyridin-3-yl)amino)pyrimidin-2-yl)-3-methylpiperidin-4-ol: Yellow solid; LCMS m/z=512 [M+H]⁺; ¹HNMR (300 MHz, DMSO-d₆) δ:10.05 (s, 1H), 9.03 (s, 1H), 8.47 (s, 1H), 8.01 (d, 1H), 7.94 (s, 1H), 6.50 (d, 1H), 4.99 (d, 1H), 4.86 (t, 1H), 4.76-4.57 (m, 3H), 4.52 (s, 1H), 3.97 (q, 1H), 3.85 (s, 1H), 3.70-3.43 (m, 3H), 3.30 (s, 6H), 3.22-2.97 (m, 3H), 1.81-1.58 (m, 2H), 1.44 (d, 3H), 1.34 (d, 3H), 1.26 (d, 3H).

Peak 2 (Example 212) (3S,4R)-3-fluoro-1-(4-((5-((R)-1-hydroxypropan-2-yl)-8-((2R,3S)-3-methoxy-2-methylazetidin-1-yl)-2,7-naphthyridin-3-yl)amino)pyrimidin-2-yl)-3-methylpiperidin-4-ol or (3S,4R)-3-fluoro-1-(4-((5-((S)-1-hydroxypropan-2-yl)-8-((2R,3S)-3-methoxy-2-methylazetidin-1-yl)-2,7-naphthyridin-3-yl)amino)pyrimidin-2-yl)-3-methylpiperidin-4-ol: Yellow solid; LCMS m/z=512 [M+H]⁺; ¹HNMR (300 MHz, DMSO-d₆) δ: 10.04 (s, 1H), 9.03 (s, 1H), 8.46 (s, 1H), 8.01 (d, 1H), 7.95 (s, 1H), 6.48 (d, 1H), 5.00 (d, 1H), 4.86 (dd, 1H), 4.72 (td, 3H), 4.57-4.48 (m, 1H), 3.96 (dd, 1H), 3.84 (dd, 1H), 3.68-3.41 (m, 3H), 3.30 (s, 3H), 3.19-2.94 (m, 3H), 1.83-1.65 (m, 2H), 1.43 (d, 3H), 1.39-1.25 (m, 6H).

### Example 213 and 214

### (3S,4R)-1-(4-((8-((R)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol and (3S,4R)-1-(4-((8-((S)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol

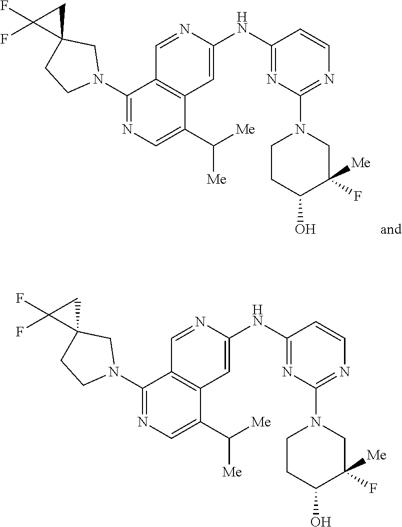 [SMILES: CC(C)c1cnc(N2CC[C@]3(C2)CC3(F)F)c2cnc(Nc3ccnc(N4CC[C@@H](O)[C@@](C)(F)C4)n3)cc12 ; "and" ; CC(C)c1cnc(N2CC[C@@]3(C2)CC3(F)F)c2cnc(Nc3ccnc(N4CC[C@@H](O)[C@@](C)(F)C4)n3)cc12]

The title compounds were prepared from (3S,4R)-1-(4-((8-(1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol (Example 63) using prep-chiral-HPLC (CHIRALPAK IF, 20×250 mm, 5 mm; 50% EtOH/Hex (8 mM NH₃/MeOH)).

Peak 1 (Example 213) (3S,4R)-1-(4-((8-((R)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol or (3S,4R)-1-(4-((8-((S)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol: LCMS m/z=528 [M+H]*; ¹HNMR (300 MHz, DMSO-d₆) δ: 10.07 (s, 1H), 9.29 (s, 1H), 8.44 (s, 1H), 8.06-7.93 (m, 2H), 6.50 (d, 1H), 5.02 (d, 1H), 4.79-4.60 (m, 2H), 3.96 (t, 2H), 3.88 (s, 2H), 3.53 (dt, 2H), 3.13 (q, 2H), 2.14 (ddt, 2H), 1.75-1.56 (m, 1H), 1.35 (d, 3H), 1.34-1.26 (m, 9H).

Peak 2 (Example 214) (3S,4R)-1-(4-((8-((R)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol or (3S,4R)-1-(4-((8-((S)-1,1-difluoro-5-azaspiro[2.4]heptan-5-yl)-5-isopropyl-2,7-naphthyridin-3-yl)amino)pyrimidin-2-yl)-3-fluoro-3-methylpiperidin-4-ol: LCMS m/z=528 [M+H]*; ¹HNMR (300 MHz, DMSO-d₆) δ: 10.05 (s, 1H), 9.28 (s, 1H), 8.44 (s, 1H), 8.05-7.94 (m, 2H), 6.50 (d, 1H), 5.02 (d, 1H), 4.79-4.60 (m, 2H), 3.96 (s, 2H), 3.88 (d, 2H), 3.53 (dt, 1H), 3.23-3.02 (m, 3H), 2.14 (tq, 2H), 1.75-1.56 (m, 4H), 1.38 (s, 1H), 1.35-1.26 (m, 8H).

### Example 215 and 216

### (3S,4R)-3-fluoro-1-(4-((5-((S)-1-methoxypropan-2-yl)-8-((R)-2-methylazetidin-1-yl)-2,7-naphthyridin-3-yl)amino)pyrimidin-2-yl)-3-methylpiperidin-4-ol and (3S,4R)-3-fluoro-1-(4-((5-((R)-1-methoxypropan-2-yl)-8-((R)-2-methylazetidin-1-yl)-2,7-naphthyridin-3-yl)amino)pyrimidin-2-yl)-3-methylpiperidin-4-ol

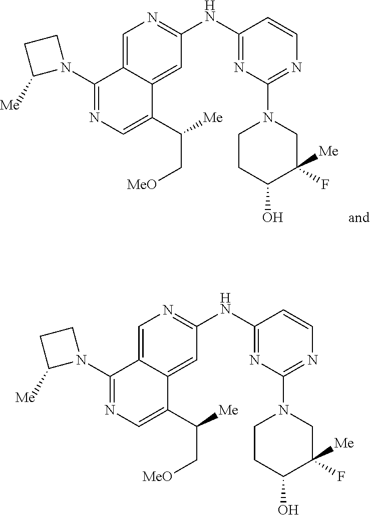 [SMILES: COC[C@@H](C)c1cnc(N2CC[C@H]2C)c2cnc(Nc3ccnc(N4CC[C@@H](O)[C@@](C)(F)C4)n3)cc12 ; "and" ; COC[C@H](C)c1cnc(N2CC[C@H]2C)c2cnc(Nc3ccnc(N4CC[C@@H](O)[C@@](C)(F)C4)n3)cc12]

The title compounds were prepared from 6-chloro-4-(1-methoxypropan-2-yl)-1-((R)-2-methylazetidin-1-yl)-2,7-naphthyridine (Preparation 300) and (3S,4R)-1-(4-aminopyrimidin-2-yl)-3-fluoro-3-methylpiperidin-4-ol (Preparation 8) using an analogous 2-part procedure as described for Example 1. Chiral-HPLC (CHIRALPAK IC, 20×250 mm, 5 mm; 50% Hex (8 mmol/L NH₃·MeOH)/EtOH) to afford the title compounds as white solids.

Peak 1 (Example 215) (3S,4R)-3-fluoro-1-(4-((5-((S)-1-methoxypropan-2-yl)-8-((R)-2-methylazetidin-1-yl)-2,7-naphthyridin-3-yl)amino)pyrimidin-2-yl)-3-methylpiperidin-4-ol or (3S,4R)-3-fluoro-1-(4-((5-((R)-1-methoxypropan-2-yl)-8-((R)-2-methylazetidin-1-yl)-2,7-naphthyridin-3-yl)amino)pyrimidin-2-yl)-3-methylpiperidin-4-ol. LCMS m/z=496 [M+H]*; ¹HNMR (300 MHz, DMSO-d₆) δ: 10.01 (s, 1H), 9.01 (s, 1H), 8.40 (s, 1H), 8.01 (d, 1H), 7.96 (s, 1H), 6.51 (d, 1H), 5.00 (d, 1H), 4.85-4.52 (m, 4H), 4.09 (q, 1H), 3.66-3.37 (m, 4H), 3.26-2.93 (m, 6H), 2.04 (d, 1H), 1.83-1.58 (m, 2H), 1.54-1.23 (m, 9H).

Peak 2 (Example 216) (3S,4R)-3-fluoro-1-(4-((5-((S)-1-methoxypropan-2-yl)-8-((R)-2-methylazetidin-1-yl)-2,7-naphthyridin-3-yl)amino)pyrimidin-2-yl)-3-methylpiperidin-4-ol or (3S,4R)-3-fluoro-1-(4-((5-((R)-1-methoxypropan-2-yl)-8-((R)-2-methylazetidin-1-yl)-2,7-naphthyridin-3-yl)amino)pyrimidin-2-yl)-3-methylpiperidin-4-ol. LCMS m/z=496 [M+H]*; ¹HNMR (300 MHz, DMSO-d₆) δ: 10.01 (s, 1H), 9.01 (s, 1H), 8.42 (s, 1H), 8.01 (d, 1H), 7.96 (s, 1H), 6.51 (d, 1H), 5.00 (d, 1H), 4.85-4.47 (m, 4H), 4.10 (q, 1H), 3.68-3.35 (m, 4H), 3.26-2.97 (m, 6H), 2.15-1.90 (m, 1H), 1.80-1.58 (m, 2H), 1.53-1.17 (m, 9H).

### Example 217

### (3S,4S)-1-(4-((5-isopropyl-8-((2R,3S)-2-methyl-3-(1,3,4-oxadiazol-2-yl)azetidin-1-yl)isoquinolin-3-yl)amino)pyrimidin-2-yl)-4-methoxypiperidin-3-ol or (3R,4R)-1-(4-((5-isopropyl-8-((2R,3S)-2-methyl-3-(1,3,4-oxadiazol-2-yl)azetidin-1-yl)isoquinolin-3-yl)amino)pyrimidin-2-yl)-4-methoxypiperidin-3-ol or (3S,4S)-1-(4-((5-isopropyl-8-((2S,3R)-2-methyl-3-(1,3,4-oxadiazol-2-yl)azetidin-1-yl)isoquinolin-3-yl)amino)pyrimidin-2-yl)-4-methoxypiperidin-3-ol or (3R,4R)-1-(4-((5-isopropyl-8-((2S,3R)-2-methyl-3-(1,3,4-oxadiazol-2-yl)azetidin-1-yl)isoquinolin-3-yl)amino)pyrimidin-2-yl)-4-methoxypiperidin-3-ol

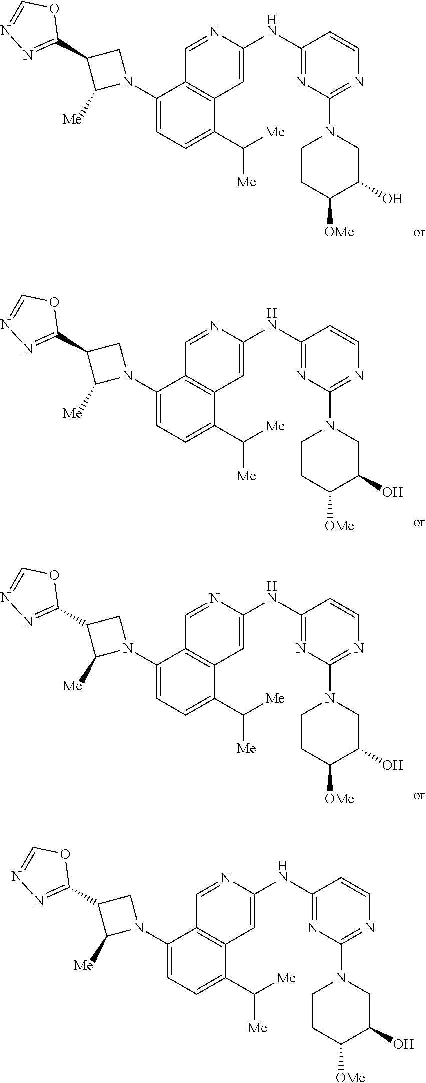

A mixture of trans-rac-2-(1-(3-chloro-5-isopropylisoquinolin-8-yl)-2-methylazetidin-3-yl)-1,3,4-oxadiazole (Preparation 118, 40 mg, 0.116 mmol), (3S,4S)-1-(4-aminopyrimidin-2-yl)-4-methoxypiperidin-3-ol or (3R,4R)-1-(4-aminopyrimidin-2-yl)-4-methoxypiperidin-3-ol (Peak 1, Preparation 18 and 19) 28.4 mg, 0.127 mmol), Brettphos Pd G3 (21 mg, 0.023 mmol) and Cs₂CO₃ (75.6 mg, 0.232 mmol) in dioxane was heated at 100° C. under N₂ for 2 h. The mixture was diluted with H₂O and extracted with EtOAc. The combined organics were dried (Na₂SO₄) and evaporated to dryness. The residue was purified by Prep-TLC with DCM/MeOH (20:1) to afford a yellow solid (20 mg, 32.5%). This material was subjected to additional purification by prep-HPLC-23 (Gradient (% organic) 19-30%) followed by prep-chiral HPLC (CHIRALPAK IA-3, 4.6×50 mm; 3 mm; 50% EtOH/(3:1 Hex/DCM+0.1% DEA) to afford the title compound as a yellow solid (5 mg, 25%).

Peak 1. LCMS m/z=531 [M+H]*; ¹HNMR (300 MHz, DMSO-d₆) δ: 9.91 (s, 1H), 9.22 (s, 1H), 9.09 (s, 1H), 8.68 (s, 1H), 7.99 (d, 1H), 7.43 (d, 1H), 6.67 (d, 1H), 6.44 (d, 1H), 5.10 (d, 1H), 4.85 (t, 1H), 4.62 (p, 1H), 4.45-4.29 (m, 2H), 3.98 (dq, 2H), 3.59-3.48 (m, 1H), 3.32 (s, 1H), 3.25-3.06 (m, 1H), 2.05 (d, 1H), 1.49 (d, 3H), 1.30 (d, 6H).

### Example 218

### (3S,4S)-1-(4-((5-isopropyl-8-((2R,3S)-2-methyl-3-(4-methyl-4H-1,2,4-triazol-3-yl)azetidin-1-yl)isoquinolin-3-yl)amino)pyrimidin-2-yl)-4-methoxypiperidin-3-ol or (3R,4R)-1-(4-((5-isopropyl-8-((2R,3S)-2-methyl-3-(4-methyl-4H-1,2,4-triazol-3-yl)azetidin-1-yl)isoquinolin-3-yl)amino)pyrimidin-2-yl)-4-methoxypiperidin-3-ol or (3S,4S)-1-(4-((5-isopropyl-8-((2S,3R)-2-methyl-3-(4-methyl-4H-1,2,4-triazol-3-yl)azetidin-1-yl)isoquinolin-3-yl)amino)pyrimidin-2-yl)-4-methoxypiperidin-3-ol or (3R,4R)-1-(4-((5-isopropyl-8-((2S,3R)-2-methyl-3-(4-methyl-4H-1,2,4-triazol-3-yl)azetidin-1-yl)isoquinolin-3-yl)amino)pyrimidin-2-yl)-4-methoxypiperidin-3-ol

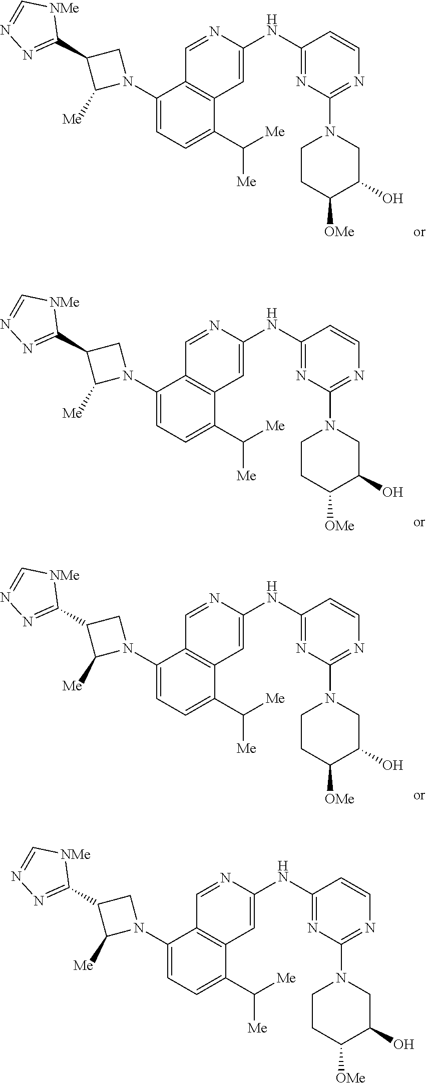

The title compound was prepared from (3S,4S)-1-(4-aminopyrimidin-2-yl)-4-methoxypiperidin-3-ol or (3R,4R)-1-(4-aminopyrimidin-2-yl)-4-methoxypiperidin-3-ol (Peak 1, Preparation 18 and 19 B28) and trans-rac-3-chloro-5-isopropyl-8-(2-methyl-3-(4-methyl-4H-1,2,4-triazol-3-yl)azetidin-1-yl)isoquinoline (Preparation 116) using an analogous method to that described for Example 217. Prep-chiral HPLC (CHIRALPAK IF-3, 4.6×50 mm; 3 mm; 30% MeOH/(3:1 Hex/DCM+8 mM NH₃/MeOH) to afford the title compound as a yellow solid (6 mg, 30%).

Peak 2. LCMS m/z=544 [M+H]⁺; ¹HNMR (300 MHz, DMSO-d₆) δ: 9.89 (s, 1H), 9.10 (s, 1H), 8.67 (s, 1H), 8.41 (s, 1H), 7.99 (d, 1H), 7.43 (d, 1H), 6.65 (d, 1H), 6.45 (d, 1H), 5.11 (s, 1H), 4.85 (t, 1H), 4.73-4.63 (m, 1H), 4.37 (s, 2H), 3.95 (t, 1H), 3.85 (q, 1H), 3.62 (s, 3H), 3.51 (d, 2H), 3.33 (s, 1H), 3.22 (s, 1H), 2.05 (d, 1H), 1.51 (d, 3H), 1.30 (m, 9H).

### Example 219

### (3S,4R)-1-(4-((8-(3-(1H-imidazol-1-yl)azetidin-1-yl)-5-isopropyl-2,7-naphthyridin-3-yl)amino)pyrimidin-2-yl)-3-fluoro-3-methylpiperidin-4-ol

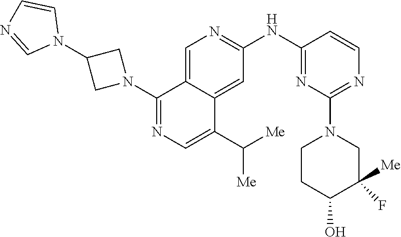 [SMILES: CC(C)c1cnc(N2CC(n3ccnc3)C2)c2cnc(Nc3ccnc(N4CC[C@@H](O)[C@@](C)(F)C4)n3)cc12]

A mixture of (3S,4R)-3-fluoro-1-(4-((5-isopropyl-8-(methylsulfonyl)-2,7-naphthyridin-3-yl)amino)pyrimidin-2-yl)-3-methylpiperidin-4-ol (Preparation 280, 10 mg, 0.021 mmol)), 1-(azetidin-3-yl)-1H-1,2,3-triazole (2.6 mg, 0.021 mmol)) and DIPEA (2.72 mg, 0.021 mmol) was heated in DMA (70 mL) at 90 C for several hours. The title compound was isolated as described in a method analogous to those described above.

Table of Compounds prepared by the synthetic methods disclosed above

TABLE 1

| Example | Structure |
|---|---|
| 1 | 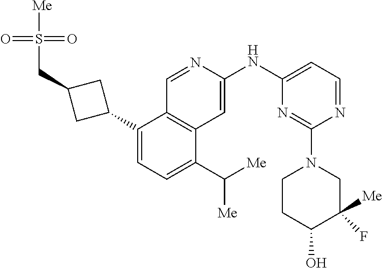 [SMILES: CC(C)c1ccc([C@H]2C[C@H](CS(C)(=O)=O)C2)c2cnc(Nc3ccnc(N4CC[C@@H](O)[C@@](C)(F)C4)n3)cc12] |
| 2 | 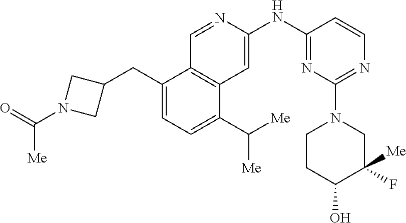 [SMILES: CC(=O)N1CC(Cc2ccc(C(C)C)c3cc(Nc4ccnc(N5CC[C@@H](O)[C@@](C)(F)C5)n4)ncc23)C1] |
| 3 | 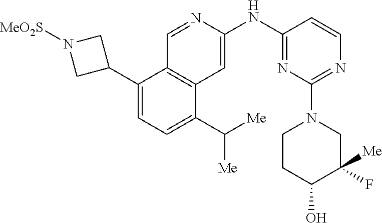 [SMILES: CC(C)c1ccc(C2CN(S(C)(=O)=O)C2)c2cnc(Nc3ccnc(N4CC[C@@H](O)[C@@](C)(F)C4)n3)cc12] |
| 4 | 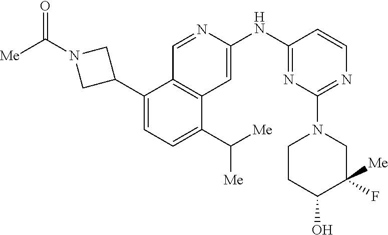 [SMILES: CC(=O)N1CC(c2ccc(C(C)C)c3cc(Nc4ccnc(N5CC[C@@H](O)[C@@](C)(F)C5)n4)ncc23)C1] |
| 5 | 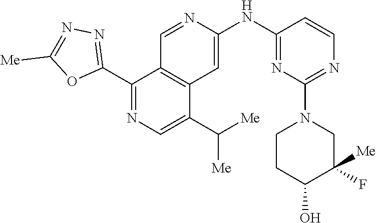 [SMILES: Cc1nnc(-c2ncc(C(C)C)c3cc(Nc4ccnc(N5CC[C@@H](O)[C@@](C)(F)C5)n4)ncc23)o1] |
| 6 | 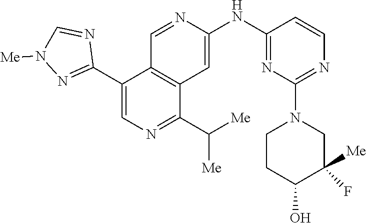 [SMILES: CC(C)c1ncc(-c2ncn(C)n2)c2cnc(Nc3ccnc(N4CC[C@@H](O)[C@@](C)(F)C4)n3)cc12] |
| 7 | 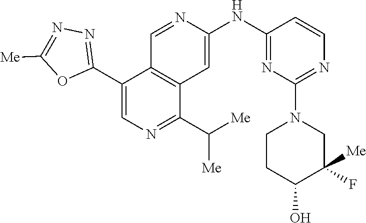 [SMILES: Cc1nnc(-c2cnc(C(C)C)c3cc(Nc4ccnc(N5CC[C@@H](O)[C@@](C)(F)C5)n4)ncc23)o1] |
| 8 | 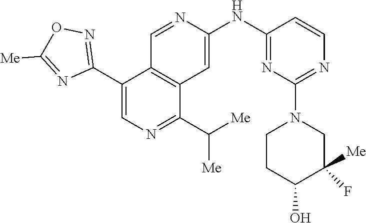 [SMILES: Cc1nc(-c2cnc(C(C)C)c3cc(Nc4ccnc(N5CC[C@@H](O)[C@@](C)(F)C5)n4)ncc23)no1] |
| 9 | 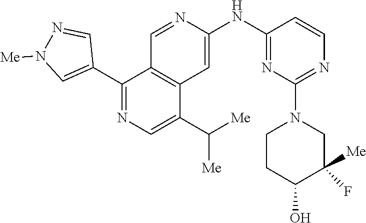 [SMILES: CC(C)c1cnc(-c2cnn(C)c2)c2cnc(Nc3ccnc(N4CC[C@@H](O)[C@@](C)(F)C4)n3)cc12] |
| 10 | 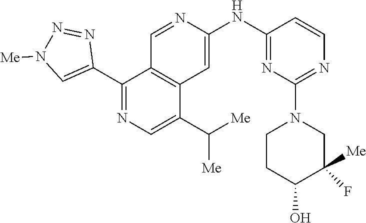 [SMILES: CC(C)c1cnc(-c2cn(C)nn2)c2cnc(Nc3ccnc(N4CC[C@@H](O)[C@@](C)(F)C4)n3)cc12] |
| 11 | 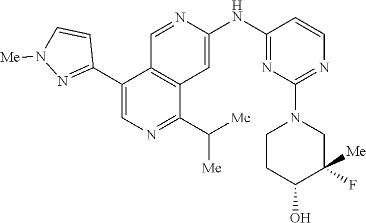 [SMILES: CC(C)c1ncc(-c2ccn(C)n2)c2cnc(Nc3ccnc(N4CC[C@@H](O)[C@@](C)(F)C4)n3)cc12] |
| 12 | 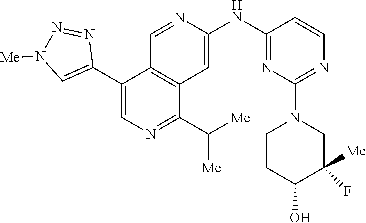 [SMILES: CC(C)c1ncc(-c2cn(C)nn2)c2cnc(Nc3ccnc(N4CC[C@@H](O)[C@@](C)(F)C4)n3)cc12] |
| 13 | 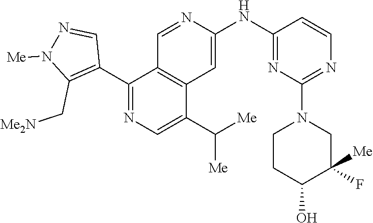 [SMILES: CC(C)c1cnc(-c2cnn(C)c2CN(C)C)c2cnc(Nc3ccnc(N4CC[C@@H](O)[C@@](C)(F)C4)n3)cc12] |
| 14 | 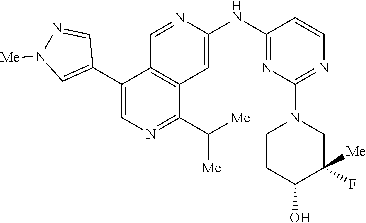 [SMILES: CC(C)c1ncc(-c2cnn(C)c2)c2cnc(Nc3ccnc(N4CC[C@@H](O)[C@@](C)(F)C4)n3)cc12] |
| 15 | 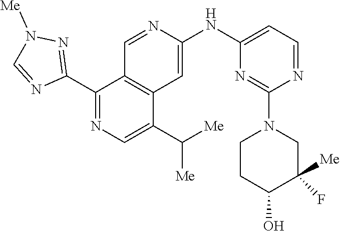 [SMILES: CC(C)c1cnc(-c2ncn(C)n2)c2cnc(Nc3ccnc(N4CC[C@@H](O)[C@@](C)(F)C4)n3)cc12] |
| 16 | 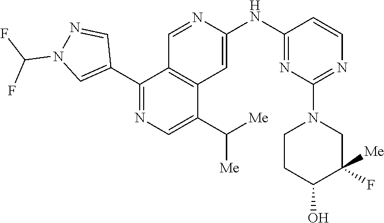 [SMILES: CC(C)c1cnc(-c2cnn(C(F)F)c2)c2cnc(Nc3ccnc(N4CC[C@@H](O)[C@@](C)(F)C4)n3)cc12] |
| 17 | 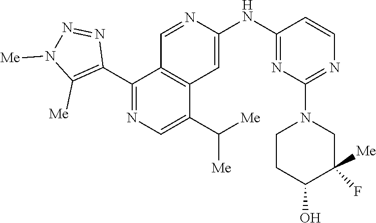 [SMILES: Cc1c(-c2ncc(C(C)C)c3cc(Nc4ccnc(N5CC[C@@H](O)[C@@](C)(F)C5)n4)ncc23)nnn1C] |
| 18 | 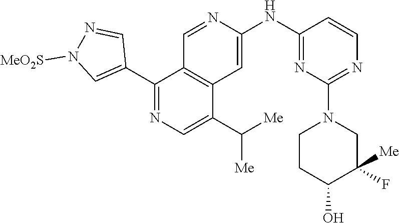 [SMILES: CC(C)c1cnc(-c2cnn(S(C)(=O)=O)c2)c2cnc(Nc3ccnc(N4CC[C@@H](O)[C@@](C)(F)C4)n3)cc12] |
| 20 | 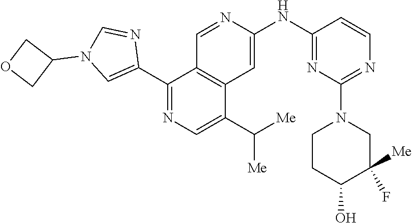 [SMILES: CC(C)c1cnc(-c2cn(C3COC3)cn2)c2cnc(Nc3ccnc(N4CC[C@@H](O)[C@@](C)(F)C4)n3)cc12] |
| 21 | 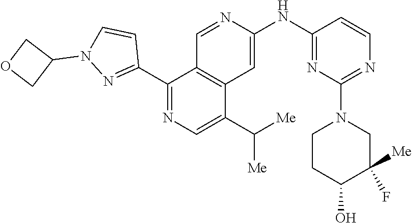 [SMILES: CC(C)c1cnc(-c2ccn(C3COC3)n2)c2cnc(Nc3ccnc(N4CC[C@@H](O)[C@@](C)(F)C4)n3)cc12] |
| 22 | 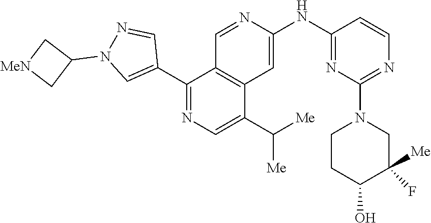 [SMILES: CC(C)c1cnc(-c2cnn(C3CN(C)C3)c2)c2cnc(Nc3ccnc(N4CC[C@@H](O)[C@@](C)(F)C4)n3)cc12] |
| 23 | 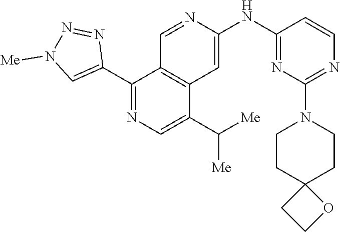 [SMILES: CC(C)c1cnc(-c2cn(C)nn2)c2cnc(Nc3ccnc(N4CCC5(CCO5)CC4)n3)cc12] |
| 24 | 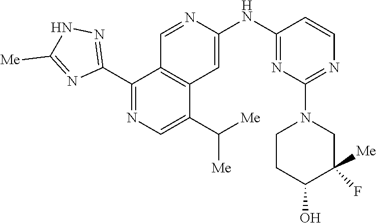 [SMILES: Cc1nc(-c2ncc(C(C)C)c3cc(Nc4ccnc(N5CC[C@@H](O)[C@@](C)(F)C5)n4)ncc23)n[nH]1] |
| 25 | 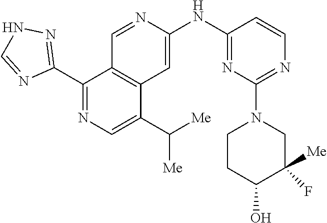 [SMILES: CC(C)c1cnc(-c2nc[nH]n2)c2cnc(Nc3ccnc(N4CC[C@@H](O)[C@@](C)(F)C4)n3)cc12] |
| 26 | 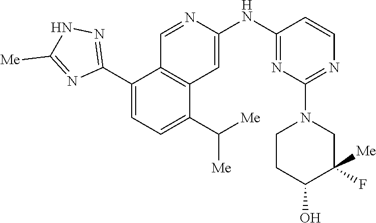 [SMILES: Cc1nc(-c2ccc(C(C)C)c3cc(Nc4ccnc(N5CC[C@@H](O)[C@@](C)(F)C5)n4)ncc23)n[nH]1] |
| 27 | 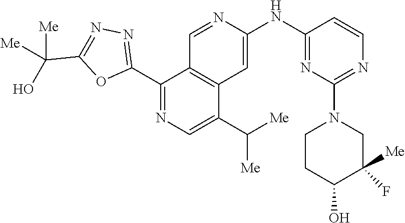 [SMILES: CC(C)c1cnc(-c2nnc(C(C)(C)O)o2)c2cnc(Nc3ccnc(N4CC[C@@H](O)[C@@](C)(F)C4)n3)cc12] |
| 28 | 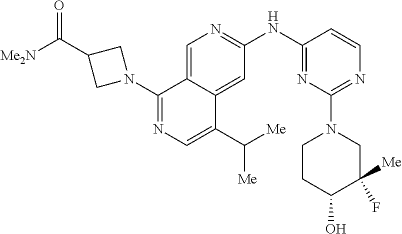 [SMILES: CC(C)c1cnc(N2CC(C(=O)N(C)C)C2)c2cnc(Nc3ccnc(N4CC[C@@H](O)[C@@](C)(F)C4)n3)cc12] |
| 29 | 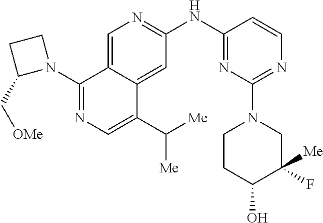 [SMILES: COC[C@@H]1CCN1c1ncc(C(C)C)c2cc(Nc3ccnc(N4CC[C@@H](O)[C@@](C)(F)C4)n3)ncc12] |
| 30 | 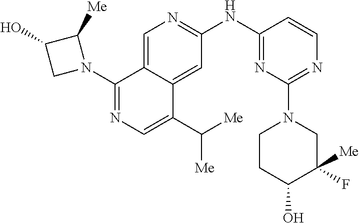 [SMILES: CC(C)c1cnc(N2C[C@H](O)[C@H]2C)c2cnc(Nc3ccnc(N4CC[C@@H](O)[C@@](C)(F)C4)n3)cc12] |
| 31 | 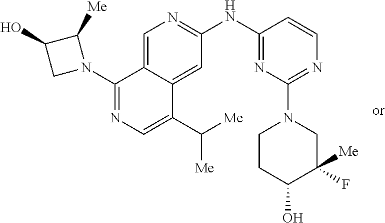 [SMILES: CC(C)c1cnc(N2C[C@@H](O)[C@H]2C)c2cnc(Nc3ccnc(N4CC[C@@H](O)[C@@](C)(F)C4)n3)cc12] |
| | 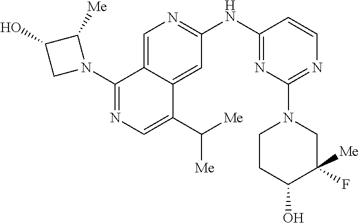 [SMILES: CC(C)c1cnc(N2C[C@H](O)[C@@H]2C)c2cnc(Nc3ccnc(N4CC[C@@H](O)[C@@](C)(F)C4)n3)cc12] |
| 32 | 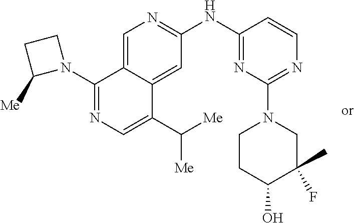 [SMILES: CC(C)c1cnc(N2CC[C@@H]2C)c2cnc(Nc3ccnc(N4CC[C@@H](O)[C@@](C)(F)C4)n3)cc12] |
| | 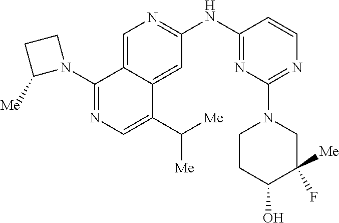 [SMILES: CC(C)c1cnc(N2CC[C@H]2C)c2cnc(Nc3ccnc(N4CC[C@@H](O)[C@@](C)(F)C4)n3)cc12] |
| 33 | 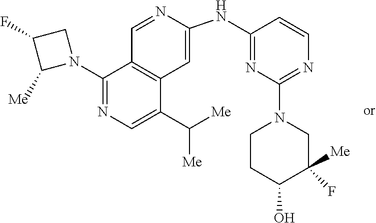 [SMILES: CC(C)c1cnc(N2C[C@@H](F)[C@H]2C)c2cnc(Nc3ccnc(N4CC[C@@H](O)[C@@](C)(F)C4)n3)cc12] |
| | 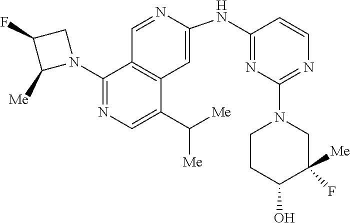 [SMILES: CC(C)c1cnc(N2C[C@H](F)[C@@H]2C)c2cnc(Nc3ccnc(N4CC[C@@H](O)[C@@](C)(F)C4)n3)cc12] |
| 34 | 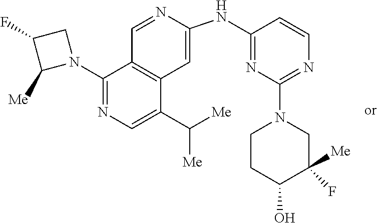 [SMILES: CC(C)c1cnc(N2C[C@@H](F)[C@@H]2C)c2cnc(Nc3ccnc(N4CC[C@@H](O)[C@@](C)(F)C4)n3)cc12] |
| | 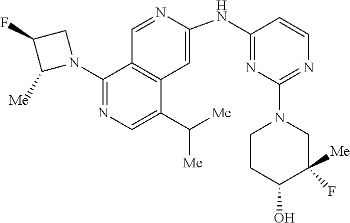 [SMILES: CC(C)c1cnc(N2C[C@H](F)[C@H]2C)c2cnc(Nc3ccnc(N4CC[C@@H](O)[C@@](C)(F)C4)n3)cc12] |
| 35 | 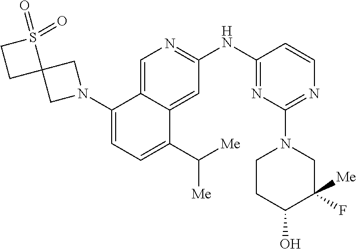 [SMILES: CC(C)c1ccc(N2CC3(CCS3(=O)=O)C2)c2cnc(Nc3ccnc(N4CC[C@@H](O)[C@@](C)(F)C4)n3)cc12] |
| 36 | 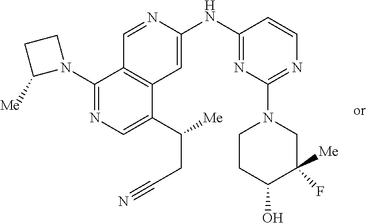 [SMILES: C[C@H](CC#N)c1cnc(N2CC[C@H]2C)c2cnc(Nc3ccnc(N4CC[C@@H](O)[C@@](C)(F)C4)n3)cc12] |
| | 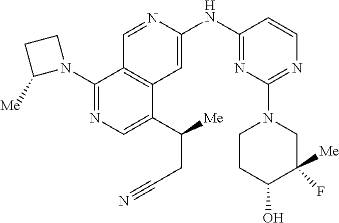 [SMILES: C[C@@H]1CCN1c1ncc([C@@H](C)CC#N)c2cc(Nc3ccnc(N4CC[C@@H](O)[C@@](C)(F)C4)n3)ncc12] |
| 37 | 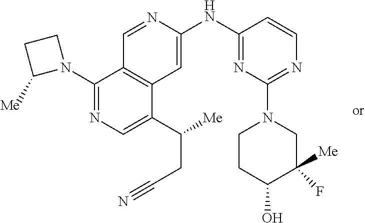 [SMILES: C[C@H](CC#N)c1cnc(N2CC[C@H]2C)c2cnc(Nc3ccnc(N4CC[C@@H](O)[C@@](C)(F)C4)n3)cc12] |
| | 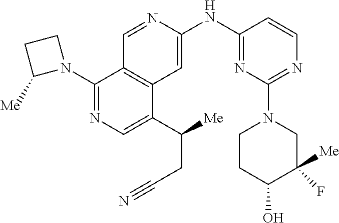 [SMILES: C[C@@H]1CCN1c1ncc([C@@H](C)CC#N)c2cc(Nc3ccnc(N4CC[C@@H](O)[C@@](C)(F)C4)n3)ncc12] |
| 38 | 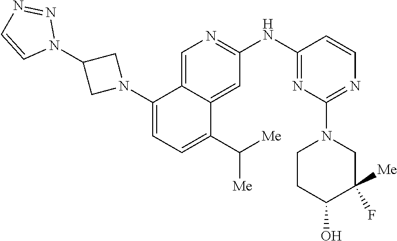 [SMILES: CC(C)c1ccc(N2CC(n3ccnn3)C2)c2cnc(Nc3ccnc(N4CC[C@@H](O)[C@@](C)(F)C4)n3)cc12] |
| 39 | 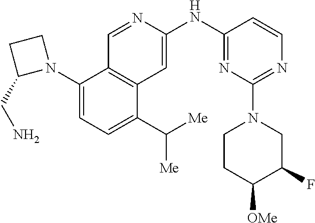 [SMILES: CO[C@H]1CCN(c2nccc(Nc3cc4c(C(C)C)ccc(N5CC[C@H]5CN)c4cn3)n2)C[C@H]1F] |
| 40 | 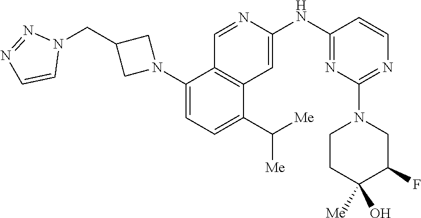 [SMILES: CC(C)c1ccc(N2CC(Cn3ccnn3)C2)c2cnc(Nc3ccnc(N4CC[C@](C)(O)[C@H](F)C4)n3)cc12] |
| 41 | 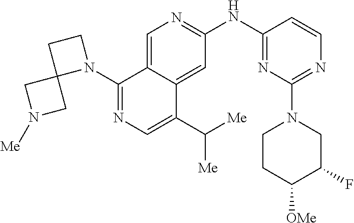 [SMILES: CO[C@@H]1CCN(c2nccc(Nc3cc4c(C(C)C)cnc(N5CCC56CN(C)C6)c4cn3)n2)C[C@@H]1F] |
| 42 | 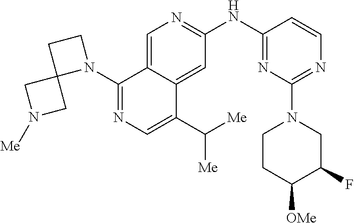 [SMILES: CO[C@H]1CCN(c2nccc(Nc3cc4c(C(C)C)cnc(N5CCC56CN(C)C6)c4cn3)n2)C[C@H]1F] |
| 43 | 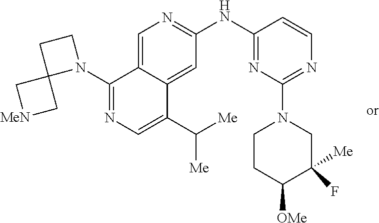 [SMILES: CO[C@H]1CCN(c2nccc(Nc3cc4c(C(C)C)cnc(N5CCC56CN(C)C6)c4cn3)n2)C[C@@]1(C)F] |
| | 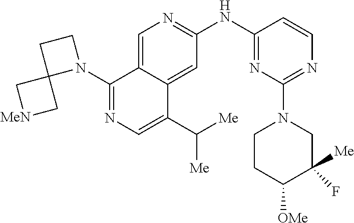 [SMILES: CO[C@@H]1CCN(c2nccc(Nc3cc4c(C(C)C)cnc(N5CCC56CN(C)C6)c4cn3)n2)C[C@]1(C)F] |
| 44 | 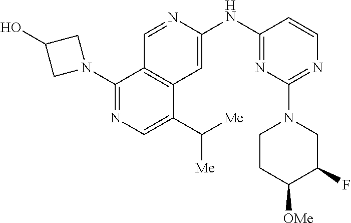 [SMILES: CO[C@H]1CCN(c2nccc(Nc3cc4c(C(C)C)cnc(N5CC(O)C5)c4cn3)n2)C[C@H]1F] |
| 45 | 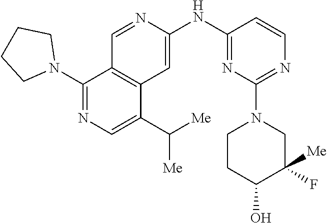 [SMILES: CC(C)c1cnc(N2CCCC2)c2cnc(Nc3ccnc(N4CC[C@@H](O)[C@@](C)(F)C4)n3)cc12] |
| 46 | 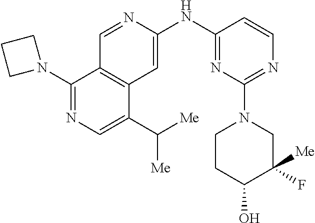 [SMILES: CC(C)c1cnc(N2CCC2)c2cnc(Nc3ccnc(N4CC[C@@H](O)[C@@](C)(F)C4)n3)cc12] |
| 47 | 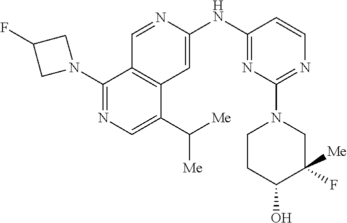 [SMILES: CC(C)c1cnc(N2CC(F)C2)c2cnc(Nc3ccnc(N4CC[C@@H](O)[C@@](C)(F)C4)n3)cc12] |
| 48 | 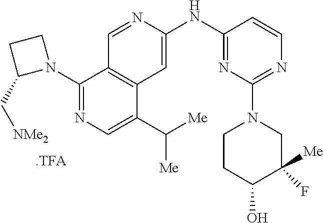 [SMILES: CC(C)c1cnc(N2CC[C@H]2CN(C)C)c2cnc(Nc3ccnc(N4CC[C@@H](O)[C@@](C)(F)C4)n3)cc12] |
| 49 | 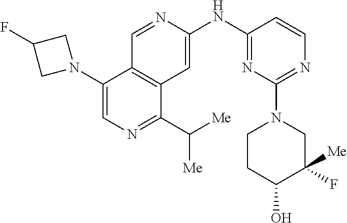 [SMILES: CC(C)c1ncc(N2CC(F)C2)c2cnc(Nc3ccnc(N4CC[C@@H](O)[C@@](C)(F)C4)n3)cc12] |
| 50 | 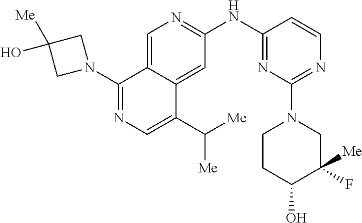 [SMILES: CC(C)c1cnc(N2CC(C)(O)C2)c2cnc(Nc3ccnc(N4CC[C@@H](O)[C@@](C)(F)C4)n3)cc12] |
| 51 | 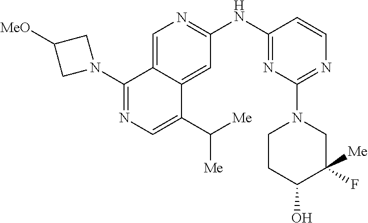 [SMILES: COC1CN(c2ncc(C(C)C)c3cc(Nc4ccnc(N5CC[C@@H](O)[C@@](C)(F)C5)n4)ncc23)C1] |
| 52 | 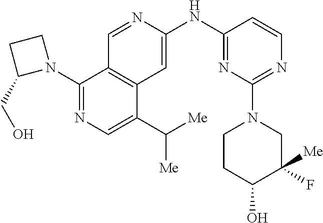 [SMILES: CC(C)c1cnc(N2CC[C@H]2CO)c2cnc(Nc3ccnc(N4CC[C@@H](O)[C@@](C)(F)C4)n3)cc12] |
| 53 | 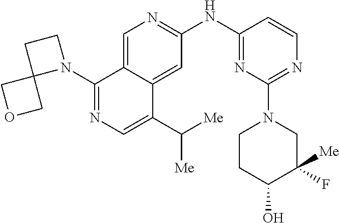 [SMILES: CC(C)c1cnc(N2CCC23COC3)c2cnc(Nc3ccnc(N4CC[C@@H](O)[C@@](C)(F)C4)n3)cc12] |
| 54 | 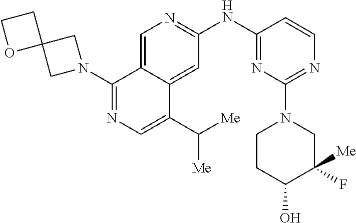 [SMILES: CC(C)c1cnc(N2CC3(CCO3)C2)c2cnc(Nc3ccnc(N4CC[C@@H](O)[C@@](C)(F)C4)n3)cc12] |
| 55 | 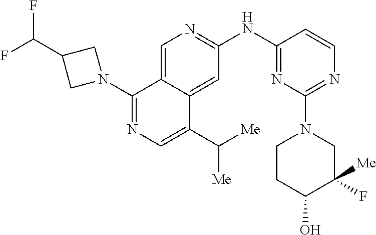 [SMILES: CC(C)c1cnc(N2CC(C(F)F)C2)c2cnc(Nc3ccnc(N4CC[C@@H](O)[C@@](C)(F)C4)n3)cc12] |
| 56 | 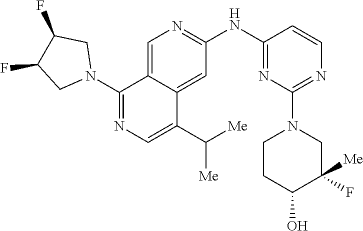 [SMILES: CC(C)c1cnc(N2C[C@@H](F)[C@@H](F)C2)c2cnc(Nc3ccnc(N4CC[C@@H](O)[C@@](C)(F)C4)n3)cc12] |
| 57 | 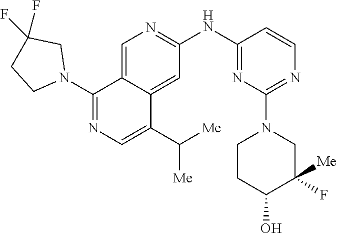 [SMILES: CC(C)c1cnc(N2CCC(F)(F)C2)c2cnc(Nc3ccnc(N4CC[C@@H](O)[C@@](C)(F)C4)n3)cc12] |
| 58 | 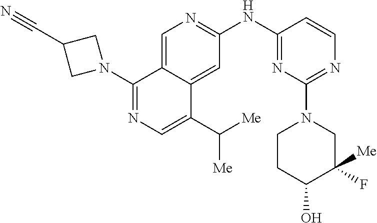 [SMILES: CC(C)c1cnc(N2CC(C#N)C2)c2cnc(Nc3ccnc(N4CC[C@@H](O)[C@@](C)(F)C4)n3)cc12] |
| 59 | 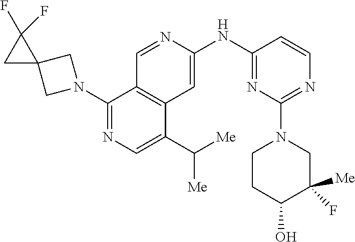 [SMILES: CC(C)c1cnc(N2CC3(C2)CC3(F)F)c2cnc(Nc3ccnc(N4CC[C@@H](O)[C@@](C)(F)C4)n3)cc12] |
| 60 | 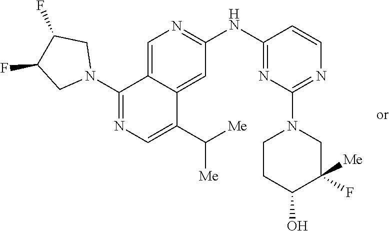 [SMILES: CC(C)c1cnc(N2C[C@@H](F)[C@H](F)C2)c2cnc(Nc3ccnc(N4CC[C@@H](O)[C@@](C)(F)C4)n3)cc12] |
| | 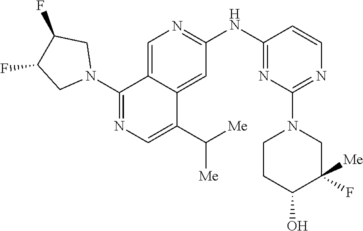 [SMILES: CC(C)c1cnc(N2C[C@H](F)[C@@H](F)C2)c2cnc(Nc3ccnc(N4CC[C@@H](O)[C@@](C)(F)C4)n3)cc12] |
| 61 | 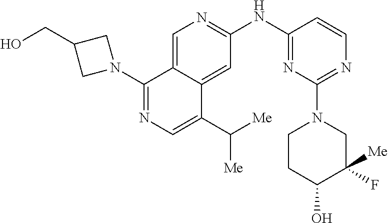 [SMILES: CC(C)c1cnc(N2CC(CO)C2)c2cnc(Nc3ccnc(N4CC[C@@H](O)[C@@](C)(F)C4)n3)cc12] |
| 62 | 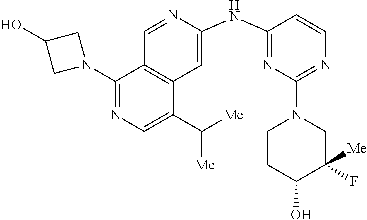 [SMILES: CC(C)c1cnc(N2CC(O)C2)c2cnc(Nc3ccnc(N4CC[C@@H](O)[C@@](C)(F)C4)n3)cc12] |
| 63 | 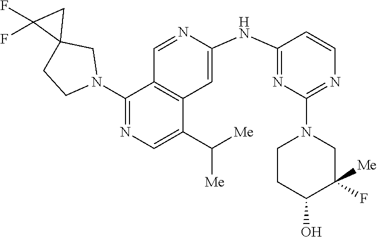 [SMILES: CC(C)c1cnc(N2CCC3(C2)CC3(F)F)c2cnc(Nc3ccnc(N4CC[C@@H](O)[C@@](C)(F)C4)n3)cc12] |
| 64 | 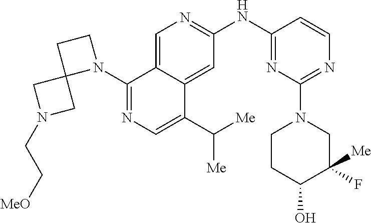 [SMILES: COCCN1CC2(CCN2c2ncc(C(C)C)c3cc(Nc4ccnc(N5CC[C@@H](O)[C@@](C)(F)C5)n4)ncc23)C1] |
| 65 | 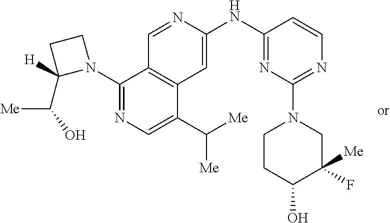 [SMILES: CC(C)c1cnc(N2CC[C@H]2[C@@H](C)O)c2cnc(Nc3ccnc(N4CC[C@@H](O)[C@@](C)(F)C4)n3)cc12] |
| | 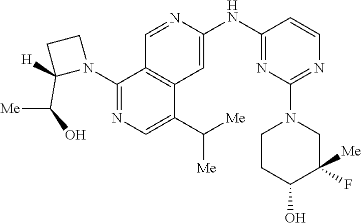 [SMILES: CC(C)c1cnc(N2CC[C@H]2[C@H](C)O)c2cnc(Nc3ccnc(N4CC[C@@H](O)[C@@](C)(F)C4)n3)cc12] |
| 66 | 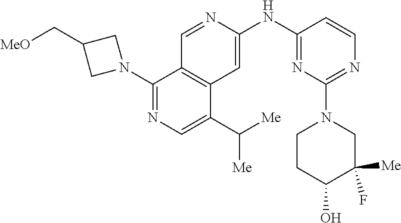 [SMILES: COCC1CN(c2ncc(C(C)C)c3cc(Nc4ccnc(N5CC[C@@H](O)[C@@](C)(F)C5)n4)ncc23)C1] |
| 67 | 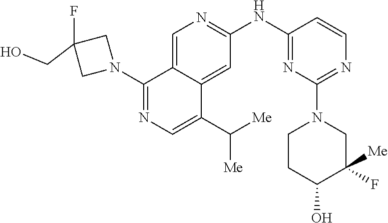 [SMILES: CC(C)c1cnc(N2CC(F)(CO)C2)c2cnc(Nc3ccnc(N4CC[C@@H](O)[C@@](C)(F)C4)n3)cc12] |
| 68 | 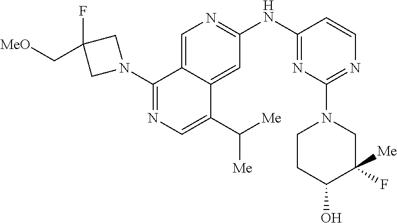 [SMILES: COCC1(F)CN(c2ncc(C(C)C)c3cc(Nc4ccnc(N5CC[C@@H](O)[C@@](C)(F)C5)n4)ncc23)C1] |
| 69 | 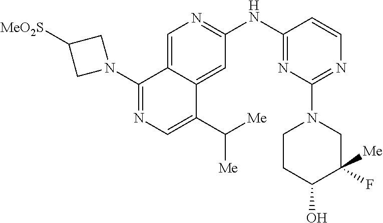 [SMILES: CC(C)c1cnc(N2CC(S(C)(=O)=O)C2)c2cnc(Nc3ccnc(N4CC[C@@H](O)[C@@](C)(F)C4)n3)cc12] |
| 70 | 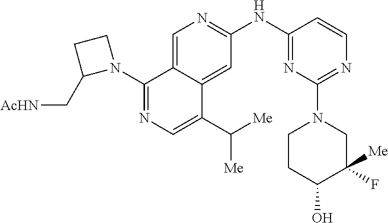 [SMILES: CC(=O)NCC1CCN1c1ncc(C(C)C)c2cc(Nc3ccnc(N4CC[C@@H](O)[C@@](C)(F)C4)n3)ncc12] |
| 71 | 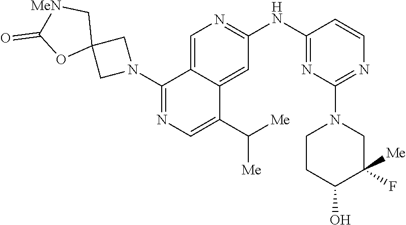 [SMILES: CC(C)c1cnc(N2CC3(CN(C)C(=O)O3)C2)c2cnc(Nc3ccnc(N4CC[C@@H](O)[C@@](C)(F)C4)n3)cc12] |
| 72 | 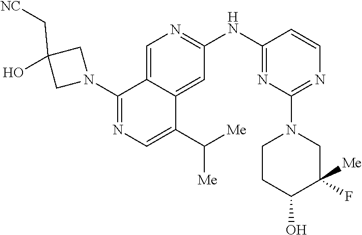 [SMILES: CC(C)c1cnc(N2CC(O)(CC#N)C2)c2cnc(Nc3ccnc(N4CC[C@@H](O)[C@@](C)(F)C4)n3)cc12] |
| 73 | 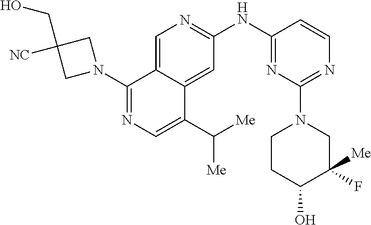 [SMILES: CC(C)c1cnc(N2CC(C#N)(CO)C2)c2cnc(Nc3ccnc(N4CC[C@@H](O)[C@@](C)(F)C4)n3)cc12] |
| 74 | 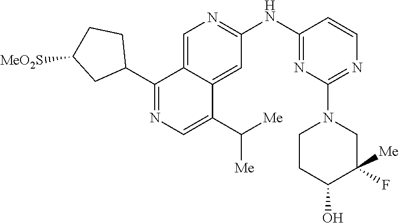 [SMILES: CC(C)c1cnc(C2CC[C@@H](S(C)(=O)=O)C2)c2cnc(Nc3ccnc(N4CC[C@@H](O)[C@@](C)(F)C4)n3)cc12] |
| 75 | 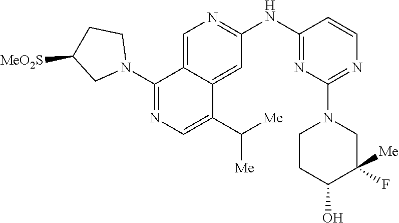 [SMILES: CC(C)c1cnc(N2CC[C@H](S(C)(=O)=O)C2)c2cnc(Nc3ccnc(N4CC[C@@H](O)[C@@](C)(F)C4)n3)cc12] |
| 76 | 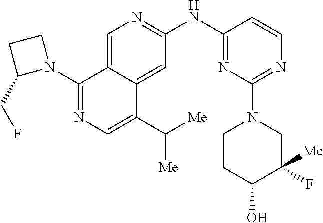 |
| 77 | 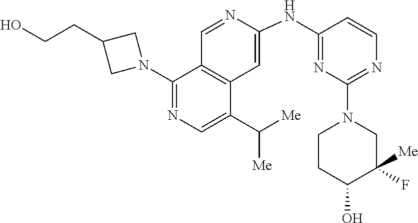 [SMILES: CC(C)c1cnc(N2CC(CCO)C2)c2cnc(Nc3ccnc(N4CC[C@@H](O)[C@@](C)(F)C4)n3)cc12] |
| 78 | 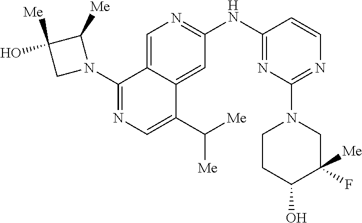 [SMILES: CC(C)c1cnc(N2C[C@](C)(O)[C@H]2C)c2cnc(Nc3ccnc(N4CC[C@@H](O)[C@@](C)(F)C4)n3)cc12] |
| 79 | 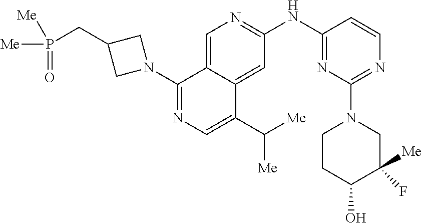 |
| 80 | 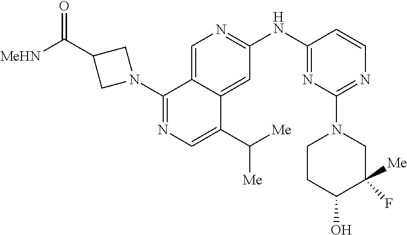 |
| 81 | 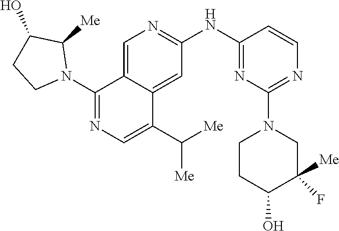 |
| 82 | 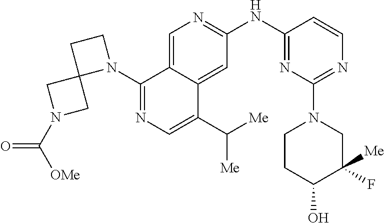 |
| 83 | 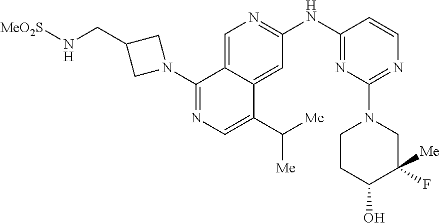 [SMILES: CC(C)c1cnc(N2CC(CNS(C)(=O)=O)C2)c2cnc(Nc3ccnc(N4CC[C@@H](O)[C@@](C)(F)C4)n3)cc12] |
| 84 | 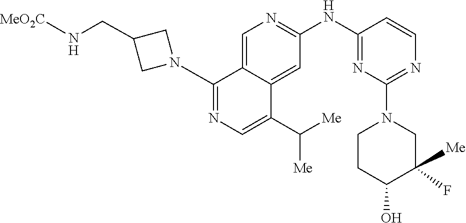 [SMILES: COC(=O)NCC1CN(c2ncc(C(C)C)c3cc(Nc4ccnc(N5CC[C@@H](O)[C@@](C)(F)C5)n4)ncc23)C1] |
| 85 | 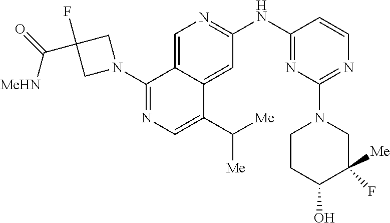 [SMILES: CNC(=O)C1(F)CN(c2ncc(C(C)C)c3cc(Nc4ccnc(N5CC[C@@H](O)[C@@](C)(F)C5)n4)ncc23)C1] |
| 86 | 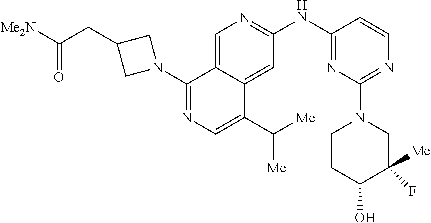 [SMILES: CC(C)c1cnc(N2CC(CC(=O)N(C)C)C2)c2cnc(Nc3ccnc(N4CC[C@@H](O)[C@@](C)(F)C4)n3)cc12] |
| 87 | 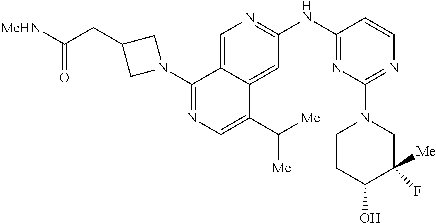 [SMILES: CNC(=O)CC1CN(c2ncc(C(C)C)c3cc(Nc4ccnc(N5CC[C@@H](O)[C@@](C)(F)C5)n4)ncc23)C1] |
| 88 | 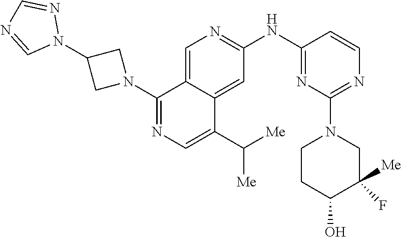 [SMILES: CC(C)c1cnc(N2CC(n3cncn3)C2)c2cnc(Nc3ccnc(N4CC[C@@H](O)[C@@](C)(F)C4)n3)cc12] |
| 89 | 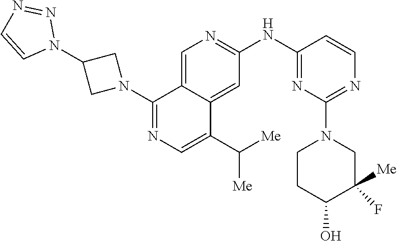 [SMILES: CC(C)c1cnc(N2CC(n3ccnn3)C2)c2cnc(Nc3ccnc(N4CC[C@@H](O)[C@@](C)(F)C4)n3)cc12] |
| 90 | 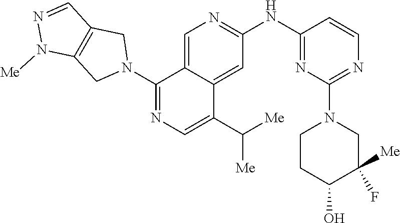 [SMILES: CC(C)c1cnc(N2Cc3cnn(C)c3C2)c2cnc(Nc3ccnc(N4CC[C@@H](O)[C@@](C)(F)C4)n3)cc12] |
| 91 | 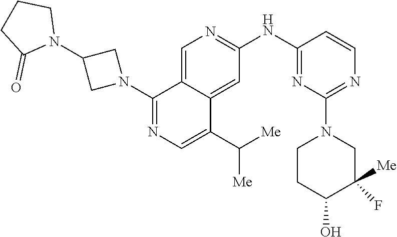 [SMILES: CC(C)c1cnc(N2CC(N3CCCC3=O)C2)c2cnc(Nc3ccnc(N4CC[C@@H](O)[C@@](C)(F)C4)n3)cc12] |
| 92 | 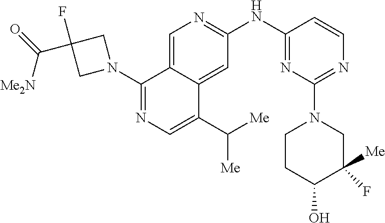 [SMILES: CC(C)c1cnc(N2CC(F)(C(=O)N(C)C)C2)c2cnc(Nc3ccnc(N4CC[C@@H](O)[C@@](C)(F)C4)n3)cc12] |
| 93 | 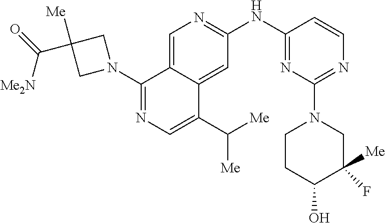 [SMILES: CC(C)c1cnc(N2CC(C)(C(=O)N(C)C)C2)c2cnc(Nc3ccnc(N4CC[C@@H](O)[C@@](C)(F)C4)n3)cc12] |
| 94 | 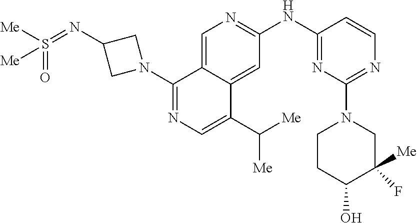 [SMILES: CC(C)c1cnc(N2CC(N=S(C)(C)=O)C2)c2cnc(Nc3ccnc(N4CC[C@@H](O)[C@@](C)(F)C4)n3)cc12] |
| 95 | 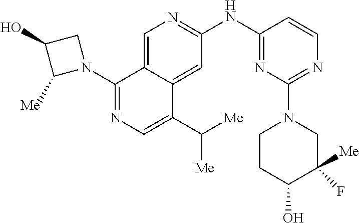 |
| 96 | 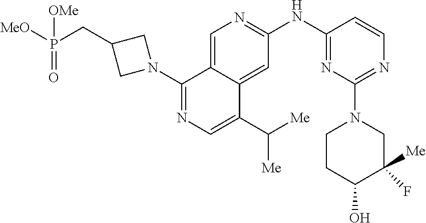 [SMILES: COP(=O)(CC1CN(c2ncc(C(C)C)c3cc(Nc4ccnc(N5CC[C@@H](O)[C@@](C)(F)C5)n4)ncc23)C1)OC] |
| 97 | 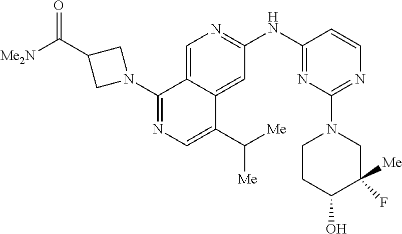 [SMILES: CC(C)c1cnc(N2CC(C(=O)N(C)C)C2)c2cnc(Nc3ccnc(N4CC[C@@H](O)[C@@](C)(F)C4)n3)cc12] |
| 98 | 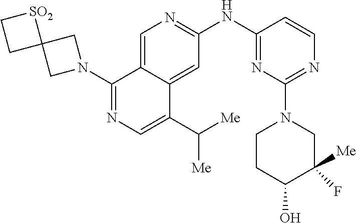 |
| 99 | 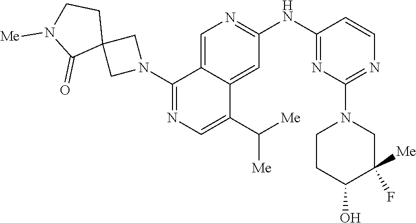 [SMILES: CC(C)c1cnc(N2CC3(CCN(C)C3=O)C2)c2cnc(Nc3ccnc(N4CC[C@@H](O)[C@@](C)(F)C4)n3)cc12] |
| 100 | 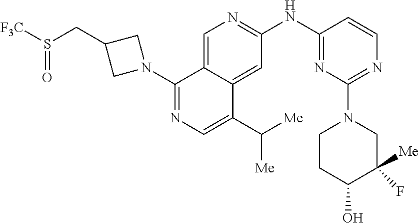 [SMILES: CC(C)c1cnc(N2CC(CS(=O)C(F)(F)F)C2)c2cnc(Nc3ccnc(N4CC[C@@H](O)[C@@](C)(F)C4)n3)cc12] |
| 101 | 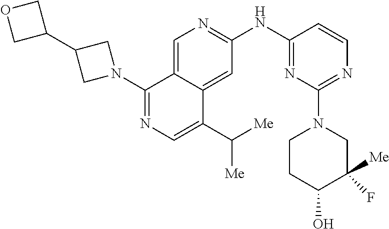 [SMILES: CC(C)c1cnc(N2CC(C3COC3)C2)c2cnc(Nc3ccnc(N4CC[C@@H](O)[C@@](C)(F)C4)n3)cc12] |
| 102 | 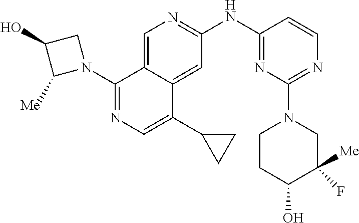 |
| 103 | 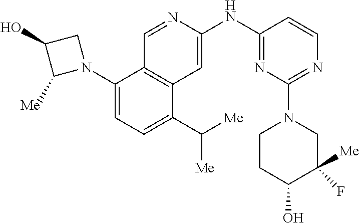 |
| 104 | 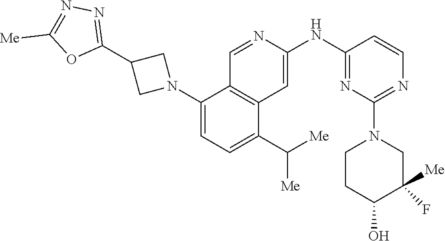 [SMILES: Cc1nnc(C2CN(c3ccc(C(C)C)c4cc(Nc5ccnc(N6CC[C@@H](O)[C@@](C)(F)C6)n5)ncc34)C2)o1] |
| 105 | 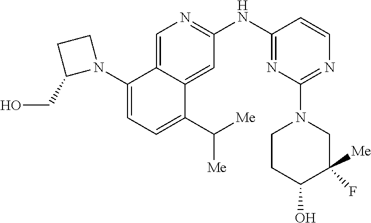 |
| 106 | 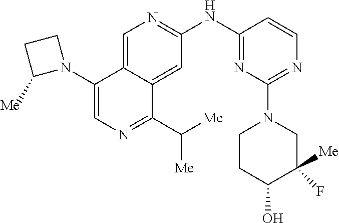 |
| 107 | 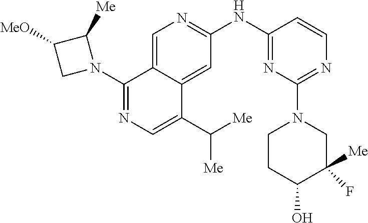 |
| 108 | 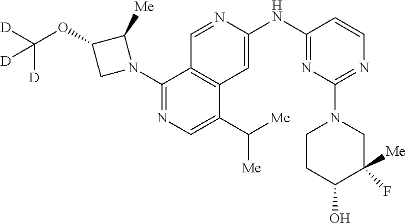 |
| 109 | 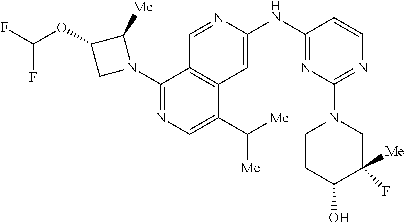 |
| 110 | 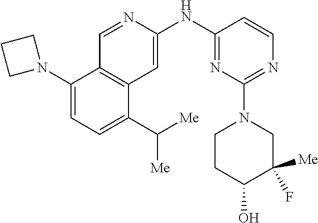 |
| 111 | 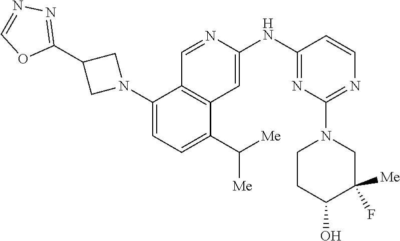 |
| 112 | 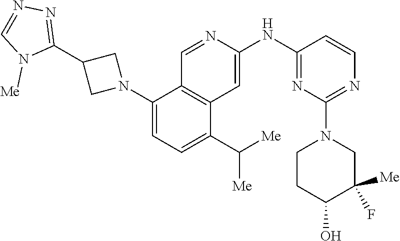 |
| 113 | 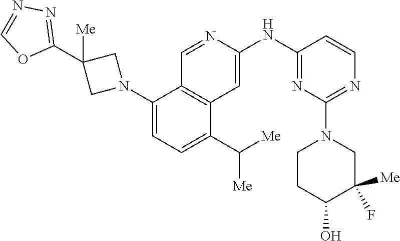 |
| 114 | 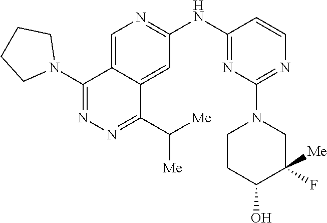 |
| 115 | 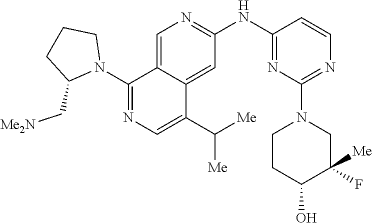 |
| 116 | 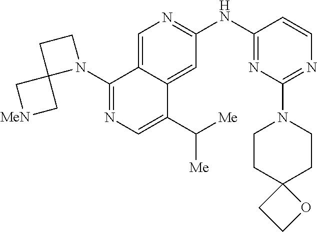 [SMILES: CC(C)c1cnc(N2CCC23CN(C)C3)c2cnc(Nc3ccnc(N4CCC5(CCO5)CC4)n3)cc12] |
| 117 | 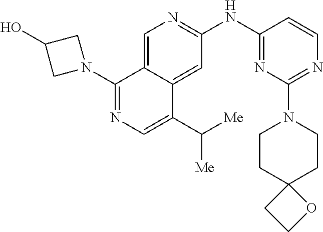 [SMILES: CC(C)c1cnc(N2CC(O)C2)c2cnc(Nc3ccnc(N4CCC5(CCO5)CC4)n3)cc12] |
| 118 | 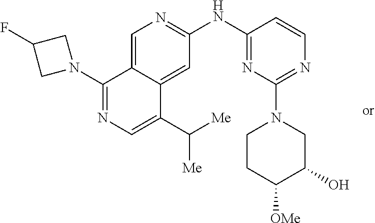 [SMILES: CO[C@@H]1CCN(c2nccc(Nc3cc4c(C(C)C)cnc(N5CC(F)C5)c4cn3)n2)C[C@@H]1O] |
| | 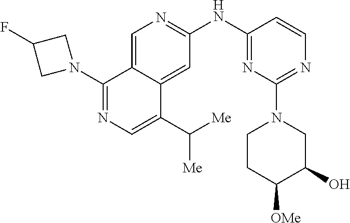 [SMILES: CO[C@H]1CCN(c2nccc(Nc3cc4c(C(C)C)cnc(N5CC(F)C5)c4cn3)n2)C[C@H]1O] |
| 119 | 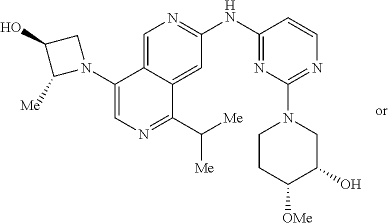 [SMILES: CO[C@@H]1CCN(c2nccc(Nc3cc4c(C(C)C)ncc(N5C[C@H](O)[C@H]5C)c4cn3)n2)C[C@@H]1O] |
| | 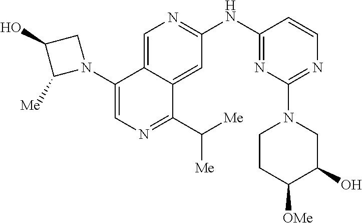 [SMILES: CO[C@H]1CCN(c2nccc(Nc3cc4c(C(C)C)ncc(N5C[C@H](O)[C@H]5C)c4cn3)n2)C[C@H]1O] |
| 120 | 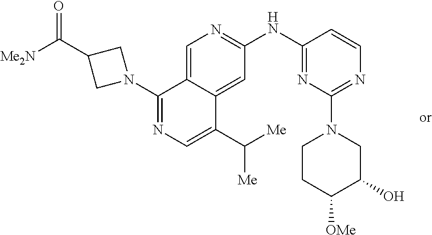 [SMILES: CO[C@@H]1CCN(c2nccc(Nc3cc4c(C(C)C)cnc(N5CC(C(=O)N(C)C)C5)c4cn3)n2)C[C@@H]1O] |
| | 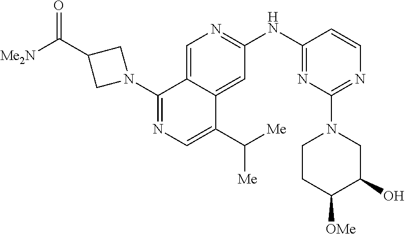 [SMILES: CO[C@H]1CCN(c2nccc(Nc3cc4c(C(C)C)cnc(N5CC(C(=O)N(C)C)C5)c4cn3)n2)C[C@H]1O] |
| 121 | 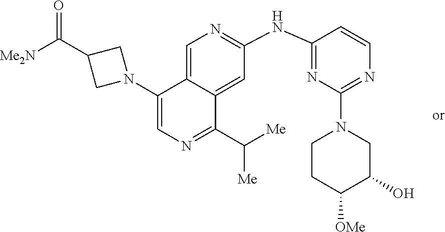 [SMILES: CO[C@@H]1CCN(c2nccc(Nc3cc4c(C(C)C)ncc(N5CC(C(=O)N(C)C)C5)c4cn3)n2)C[C@@H]1O] |
| | 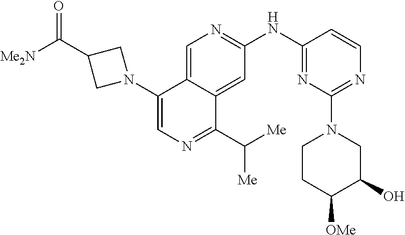 [SMILES: CO[C@H]1CCN(c2nccc(Nc3cc4c(C(C)C)ncc(N5CC(C(=O)N(C)C)C5)c4cn3)n2)C[C@H]1O] |
| 122 | 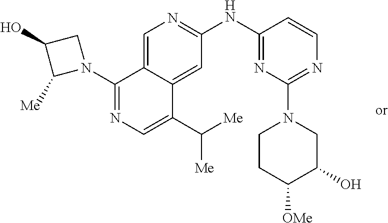 [SMILES: CO[C@@H]1CCN(c2nccc(Nc3cc4c(C(C)C)cnc(N5C[C@H](O)[C@H]5C)c4cn3)n2)C[C@@H]1O] |
| | 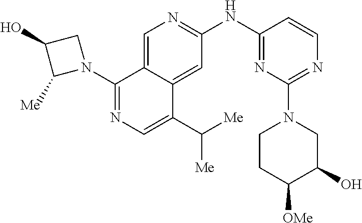 [SMILES: CO[C@H]1CCN(c2nccc(Nc3cc4c(C(C)C)cnc(N5C[C@H](O)[C@H]5C)c4cn3)n2)C[C@H]1O] |
| 123 | 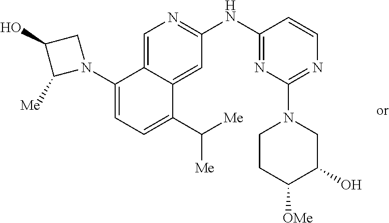 [SMILES: CO[C@@H]1CCN(c2nccc(Nc3cc4c(C(C)C)ccc(N5C[C@H](O)[C@H]5C)c4cn3)n2)C[C@@H]1O] |
| | 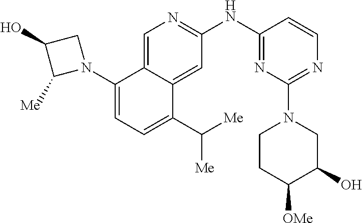 [SMILES: CO[C@H]1CCN(c2nccc(Nc3cc4c(C(C)C)ccc(N5C[C@H](O)[C@H]5C)c4cn3)n2)C[C@H]1O] |
| 124 | 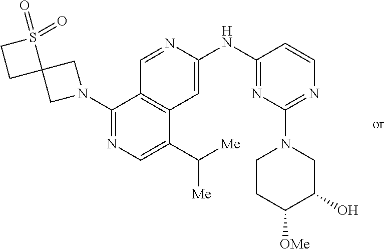 [SMILES: CO[C@@H]1CCN(c2nccc(Nc3cc4c(C(C)C)cnc(N5CC6(CCS6(=O)=O)C5)c4cn3)n2)C[C@@H]1O] |
| | 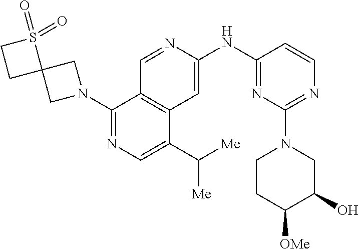 [SMILES: CO[C@H]1CCN(c2nccc(Nc3cc4c(C(C)C)cnc(N5CC6(CCS6(=O)=O)C5)c4cn3)n2)C[C@H]1O] |
| 125 | 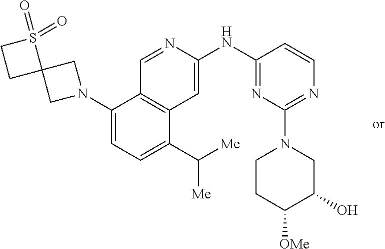 [SMILES: CO[C@@H]1CCN(c2nccc(Nc3cc4c(C(C)C)ccc(N5CC6(CCS6(=O)=O)C5)c4cn3)n2)C[C@@H]1O] |
| | 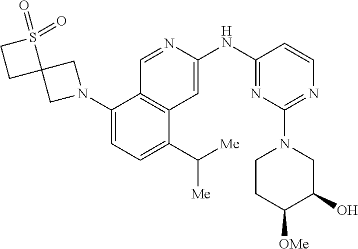 [SMILES: CO[C@H]1CCN(c2nccc(Nc3cc4c(C(C)C)ccc(N5CC6(CCS6(=O)=O)C5)c4cn3)n2)C[C@H]1O] |
| 126 | 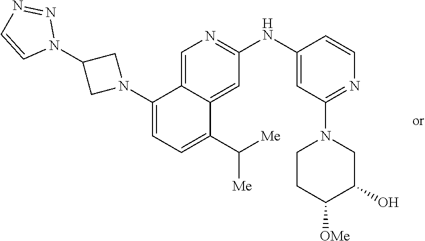 [SMILES: CO[C@@H]1CCN(c2cc(Nc3cc4c(C(C)C)ccc(N5CC(n6ccnn6)C5)c4cn3)ccn2)C[C@@H]1O] |
| | 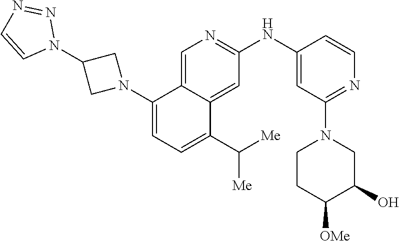 [SMILES: CO[C@H]1CCN(c2cc(Nc3cc4c(C(C)C)ccc(N5CC(n6ccnn6)C5)c4cn3)ccn2)C[C@H]1O] |
| 127 | 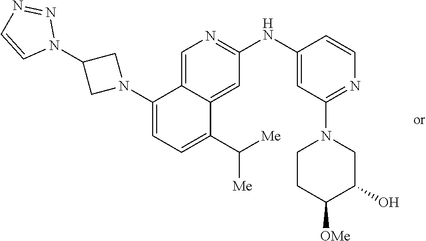 [SMILES: CO[C@H]1CCN(c2cc(Nc3cc4c(C(C)C)ccc(N5CC(n6ccnn6)C5)c4cn3)ccn2)C[C@@H]1O] |
| | 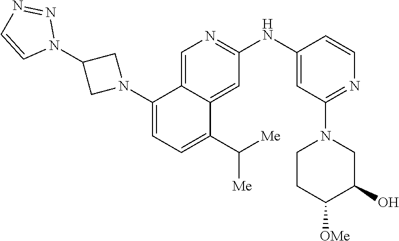 [SMILES: CO[C@@H]1CCN(c2cc(Nc3cc4c(C(C)C)ccc(N5CC(n6ccnn6)C5)c4cn3)ccn2)C[C@H]1O] |
| 128 | 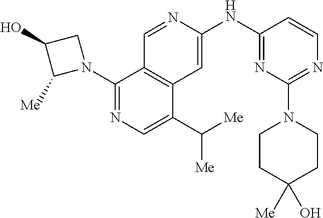 [SMILES: CC(C)c1cnc(N2C[C@H](O)[C@H]2C)c2cnc(Nc3ccnc(N4CCC(C)(O)CC4)n3)cc12] |
| 129 | 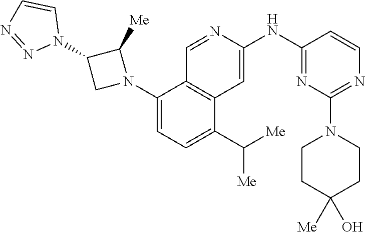 [SMILES: CC(C)c1ccc(N2C[C@H](n3ccnn3)[C@H]2C)c2cnc(Nc3ccnc(N4CCC(C)(O)CC4)n3)cc12] |
| 130 | 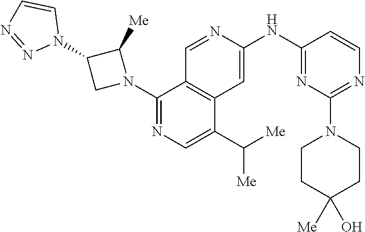 [SMILES: CC(C)c1cnc(N2C[C@H](n3ccnn3)[C@H]2C)c2cnc(Nc3ccnc(N4CCC(C)(O)CC4)n3)cc12] |
| 131 | 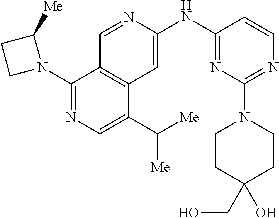 [SMILES: CC(C)c1cnc(N2CC[C@H]2C)c2cnc(Nc3ccnc(N4CCC(O)(CO)CC4)n3)cc12] |
| 132 | 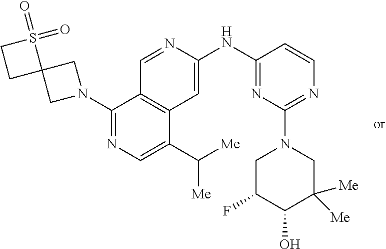 [SMILES: CC(C)c1cnc(N2CC3(CCS3(=O)=O)C2)c2cnc(Nc3ccnc(N4C[C@@H](F)[C@@H](O)C(C)(C)C4)n3)cc12] |
| | 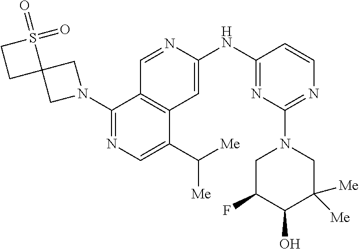 [SMILES: CC(C)c1cnc(N2CC3(CCS3(=O)=O)C2)c2cnc(Nc3ccnc(N4C[C@H](F)[C@H](O)C(C)(C)C4)n3)cc12] |
| 133 | 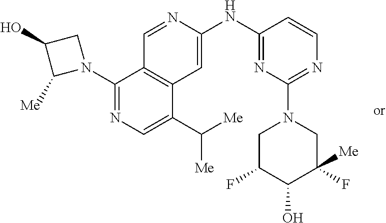 [SMILES: CC(C)c1cnc(N2C[C@H](O)[C@H]2C)c2cnc(Nc3ccnc(N4C[C@@H](F)[C@@H](O)[C@@](C)(F)C4)n3)cc12] |
| | 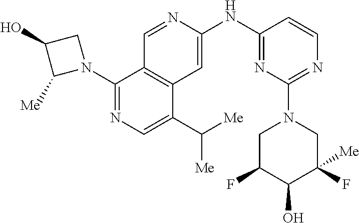 [SMILES: CC(C)c1cnc(N2C[C@H](O)[C@H]2C)c2cnc(Nc3ccnc(N4C[C@H](F)[C@H](O)[C@](C)(F)C4)n3)cc12] |
| 134 | 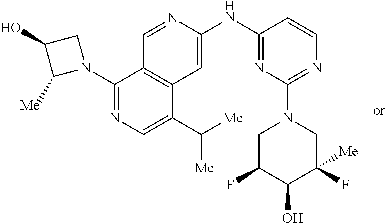 [SMILES: CC(C)c1cnc(N2C[C@H](O)[C@H]2C)c2cnc(Nc3ccnc(N4C[C@H](F)[C@H](O)[C@](C)(F)C4)n3)cc12] |
| | 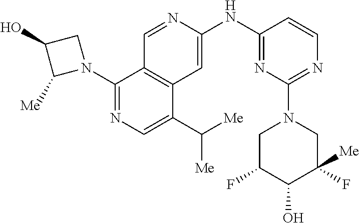 [SMILES: CC(C)c1cnc(N2C[C@H](O)[C@H]2C)c2cnc(Nc3ccnc(N4C[C@@H](F)[C@@H](O)[C@@](C)(F)C4)n3)cc12] |
| 135 | 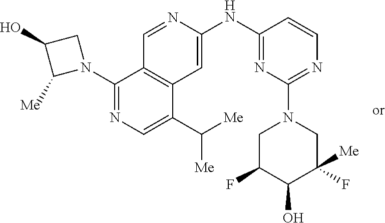 [SMILES: CC(C)c1cnc(N2C[C@H](O)[C@H]2C)c2cnc(Nc3ccnc(N4C[C@H](F)[C@H](O)[C@@](C)(F)C4)n3)cc12] |
| | 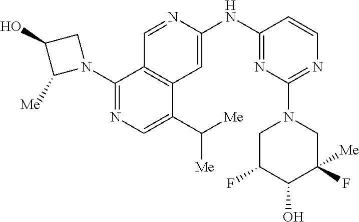 [SMILES: CC(C)c1cnc(N2C[C@H](O)[C@H]2C)c2cnc(Nc3ccnc(N4C[C@@H](F)[C@@H](O)[C@](C)(F)C4)n3)cc12] |
| 136 | 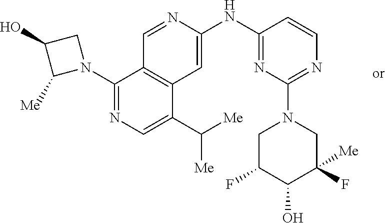 [SMILES: CC(C)c1cnc(N2C[C@H](O)[C@H]2C)c2cnc(Nc3ccnc(N4C[C@@H](F)[C@@H](O)[C@](C)(F)C4)n3)cc12] |
| | 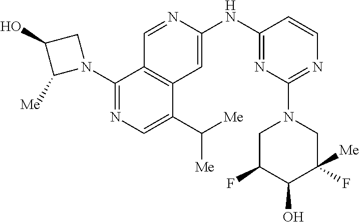 [SMILES: CC(C)c1cnc(N2C[C@H](O)[C@H]2C)c2cnc(Nc3ccnc(N4C[C@H](F)[C@H](O)[C@@](C)(F)C4)n3)cc12] |
| 137 | 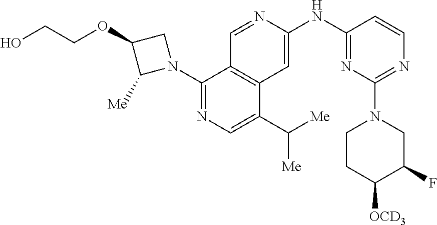 |
| 138 | 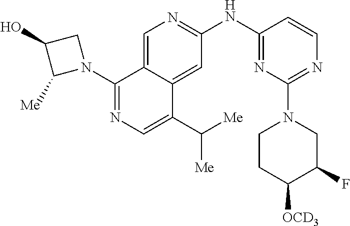 |
| 139 | 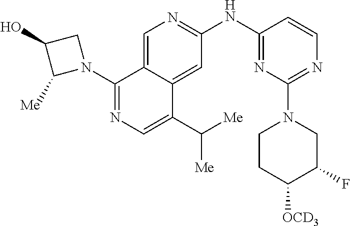 |
| 140 | 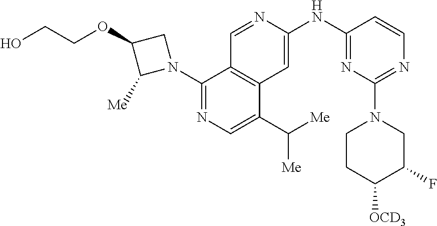 |
| 141 | 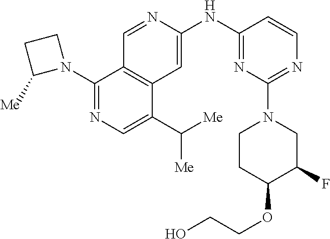 [SMILES: CC(C)c1cnc(N2CC[C@H]2C)c2cnc(Nc3ccnc(N4CC[C@H](OCCO)[C@H](F)C4)n3)cc12] |
| 142 | 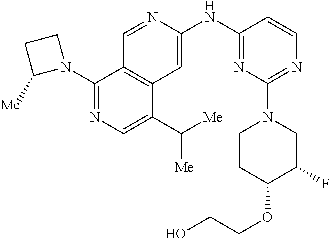 [SMILES: CC(C)c1cnc(N2CC[C@H]2C)c2cnc(Nc3ccnc(N4CC[C@@H](OCCO)[C@@H](F)C4)n3)cc12] |
| 143 | 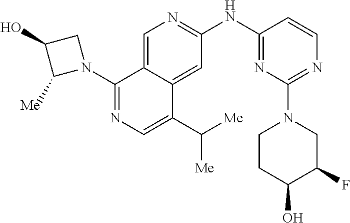 [SMILES: CC(C)c1cnc(N2C[C@H](O)[C@H]2C)c2cnc(Nc3ccnc(N4CC[C@H](O)[C@H](F)C4)n3)cc12] |
| 144 | 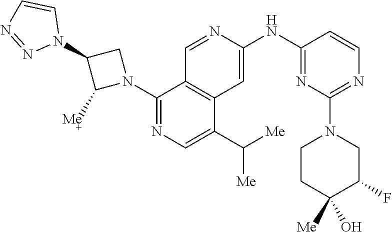 [SMILES: CC(C)c1cnc(N2C[C@H](n3ccnn3)[C@H]2C)c2cnc(Nc3ccnc(N4CC[C@@](C)(O)[C@@H](F)C4)n3)cc12] |
| 145 | 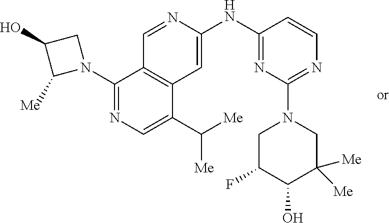 [SMILES: CC(C)c1cnc(N2C[C@H](O)[C@H]2C)c2cnc(Nc3ccnc(N4C[C@@H](F)[C@@H](O)C(C)(C)C4)n3)cc12] |
| | 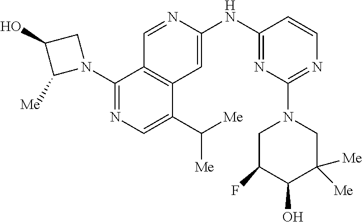 [SMILES: CC(C)c1cnc(N2C[C@H](O)[C@H]2C)c2cnc(Nc3ccnc(N4C[C@H](F)[C@H](O)C(C)(C)C4)n3)cc12] |
| 146 | 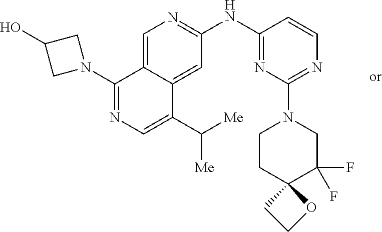 [SMILES: CC(C)c1cnc(N2CC(O)C2)c2cnc(Nc3ccnc(N4CC[C@@]5(CCO5)C(F)(F)C4)n3)cc12] |
| | 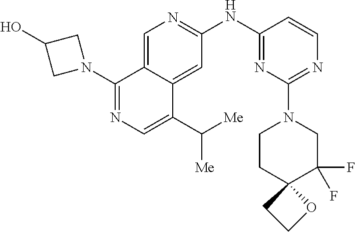 [SMILES: CC(C)c1cnc(N2CC(O)C2)c2cnc(Nc3ccnc(N4CC[C@]5(CCO5)C(F)(F)C4)n3)cc12] |
| 147 | 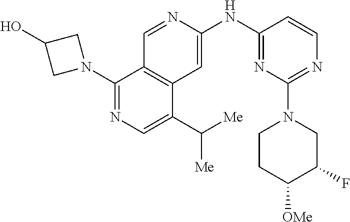 [SMILES: CO[C@@H]1CCN(c2nccc(Nc3cc4c(C(C)C)cnc(N5CC(O)C5)c4cn3)n2)C[C@@H]1F] |
| 148 | 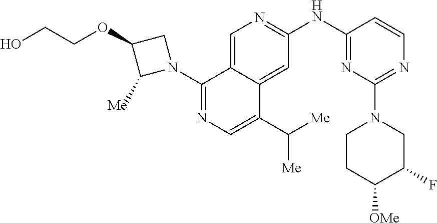 [SMILES: CO[C@@H]1CCN(c2nccc(Nc3cc4c(C(C)C)cnc(N5C[C@H](OCCO)[C@H]5C)c4cn3)n2)C[C@@H]1F] |
| 149 | 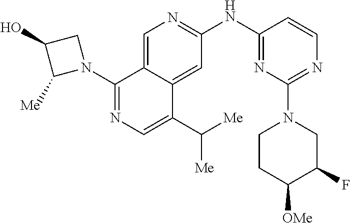 [SMILES: CO[C@H]1CCN(c2nccc(Nc3cc4c(C(C)C)cnc(N5C[C@H](O)[C@H]5C)c4cn3)n2)C[C@H]1F] |
| 150 | 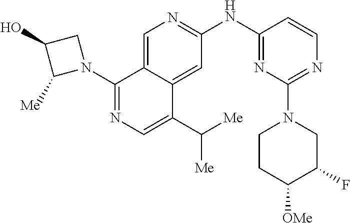 [SMILES: CO[C@@H]1CCN(c2nccc(Nc3cc4c(C(C)C)cnc(N5C[C@H](O)[C@H]5C)c4cn3)n2)C[C@@H]1F] |
| 151 | 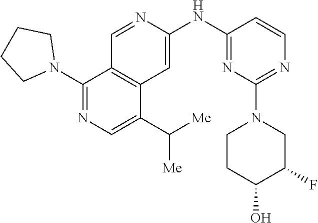 [SMILES: CC(C)c1cnc(N2CCCC2)c2cnc(Nc3ccnc(N4CC[C@@H](O)[C@@H](F)C4)n3)cc12] |
| 152 | 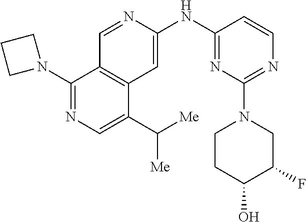 [SMILES: CC(C)c1cnc(N2CCC2)c2cnc(Nc3ccnc(N4CC[C@@H](O)[C@@H](F)C4)n3)cc12] |
| 153 | 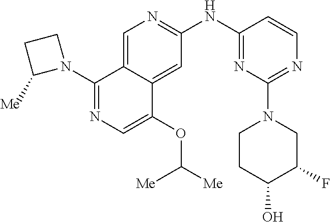 [SMILES: CC(C)Oc1cnc(N2CC[C@H]2C)c2cnc(Nc3ccnc(N4CC[C@@H](O)[C@@H](F)C4)n3)cc12] |
| 154 | 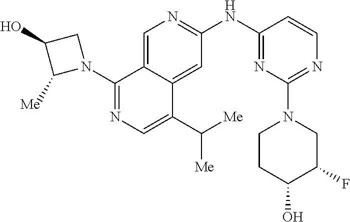 [SMILES: CC(C)c1cnc(N2C[C@H](O)[C@H]2C)c2cnc(Nc3ccnc(N4CC[C@@H](O)[C@@H](F)C4)n3)cc12] |
| 155 | 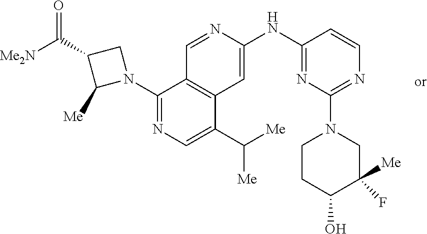 [SMILES: CC(C)c1cnc(N2C[C@@H](C(=O)N(C)C)[C@@H]2C)c2cnc(Nc3ccnc(N4CC[C@@H](O)[C@@](C)(F)C4)n3)cc12] |
| | 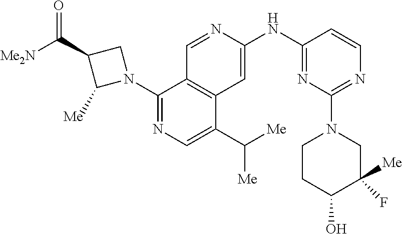 [SMILES: CC(C)c1cnc(N2C[C@H](C(=O)N(C)C)[C@H]2C)c2cnc(Nc3ccnc(N4CC[C@@H](O)[C@@](C)(F)C4)n3)cc12] |
| 156 | 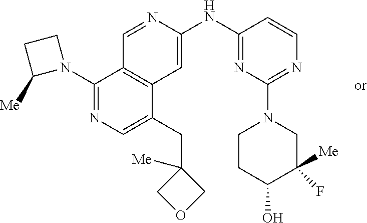 [SMILES: C[C@H]1CCN1c1ncc(CC2(C)COC2)c2cc(Nc3ccnc(N4CC[C@@H](O)[C@@](C)(F)C4)n3)ncc12] |
| | 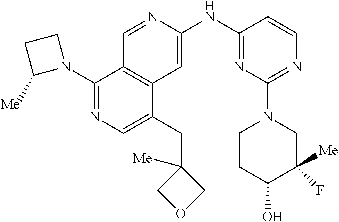 [SMILES: C[C@@H]1CCN1c1ncc(CC2(C)COC2)c2cc(Nc3ccnc(N4CC[C@@H](O)[C@@](C)(F)C4)n3)ncc12] |
| 157 | 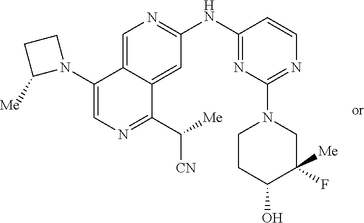 [SMILES: C[C@H](C#N)c1ncc(N2CC[C@H]2C)c2cnc(Nc3ccnc(N4CC[C@@H](O)[C@@](C)(F)C4)n3)cc12] |
| | 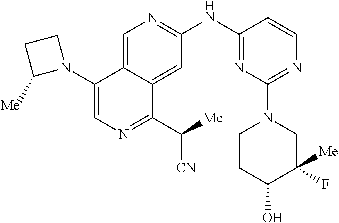 [SMILES: C[C@@H]1CCN1c1cnc([C@@H](C)C#N)c2cc(Nc3ccnc(N4CC[C@@H](O)[C@@](C)(F)C4)n3)ncc12] |
| 158 | 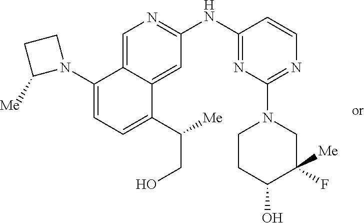 [SMILES: C[C@H](CO)c1ccc(N2CC[C@H]2C)c2cnc(Nc3ccnc(N4CC[C@@H](O)[C@@](C)(F)C4)n3)cc12] |
| | 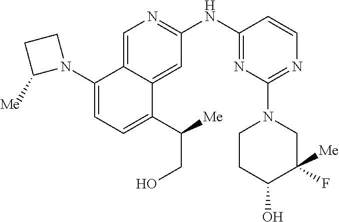 [SMILES: C[C@@H]1CCN1c1ccc([C@@H](C)CO)c2cc(Nc3ccnc(N4CC[C@@H](O)[C@@](C)(F)C4)n3)ncc12] |
| 159 | 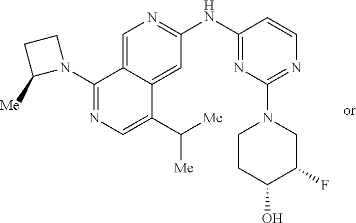 [SMILES: CC(C)c1cnc(N2CC[C@@H]2C)c2cnc(Nc3ccnc(N4CC[C@@H](O)[C@@H](F)C4)n3)cc12] |
| | 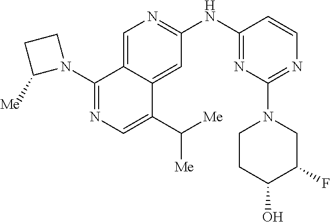 [SMILES: CC(C)c1cnc(N2CC[C@H]2C)c2cnc(Nc3ccnc(N4CC[C@@H](O)[C@@H](F)C4)n3)cc12] |
| 160 | 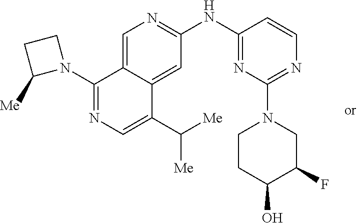 [SMILES: CC(C)c1cnc(N2CC[C@@H]2C)c2cnc(Nc3ccnc(N4CC[C@H](O)[C@H](F)C4)n3)cc12] |
| | 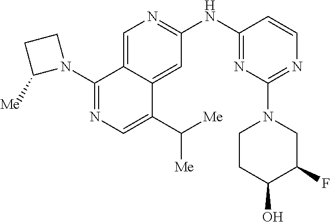 [SMILES: CC(C)c1cnc(N2CC[C@H]2C)c2cnc(Nc3ccnc(N4CC[C@H](O)[C@H](F)C4)n3)cc12] |
| 161 | 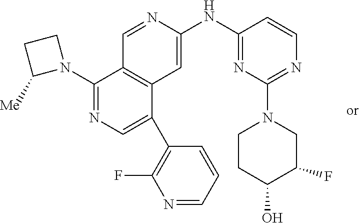 [SMILES: C[C@@H]1CCN1c1ncc(-c2cccnc2F)c2cc(Nc3ccnc(N4CC[C@@H](O)[C@@H](F)C4)n3)ncc12] |
| | 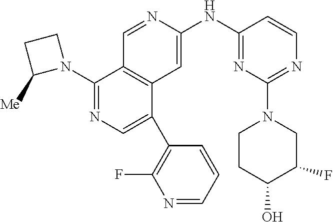 [SMILES: C[C@H]1CCN1c1ncc(-c2cccnc2F)c2cc(Nc3ccnc(N4CC[C@@H](O)[C@@H](F)C4)n3)ncc12] |
| 162 | 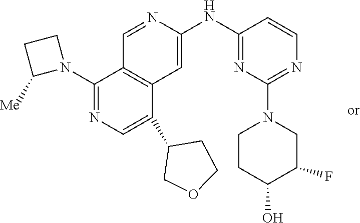 [SMILES: C[C@@H]1CCN1c1ncc([C@@H]2CCOC2)c2cc(Nc3ccnc(N4CC[C@@H](O)[C@@H](F)C4)n3)ncc12] |
| | 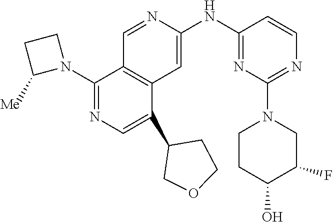 [SMILES: C[C@@H]1CCN1c1ncc([C@H]2CCOC2)c2cc(Nc3ccnc(N4CC[C@@H](O)[C@@H](F)C4)n3)ncc12] |
| 163 | 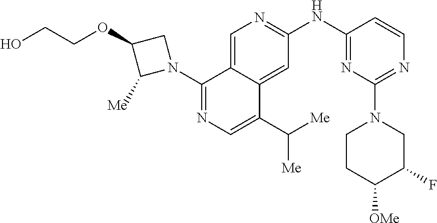 [SMILES: CO[C@@H]1CCN(c2nccc(Nc3cc4c(C(C)C)cnc(N5C[C@H](OCCO)[C@H]5C)c4cn3)n2)C[C@@H]1F] |
| 164 | 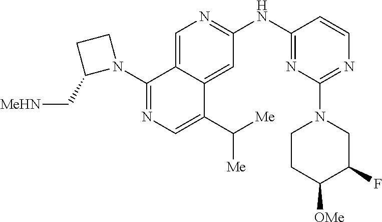 [SMILES: CNC[C@@H]1CCN1c1ncc(C(C)C)c2cc(Nc3ccnc(N4CC[C@H](OC)[C@H](F)C4)n3)ncc12] |
| 165 | 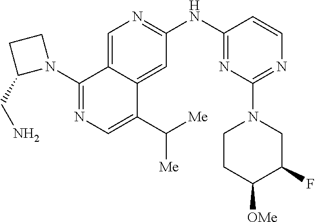 [SMILES: CO[C@H]1CCN(c2nccc(Nc3cc4c(C(C)C)cnc(N5CC[C@H]5CN)c4cn3)n2)C[C@H]1F] |
| 166 | 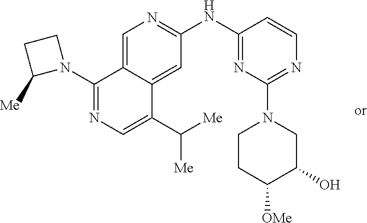 [SMILES: CO[C@@H]1CCN(c2nccc(Nc3cc4c(C(C)C)cnc(N5CC[C@@H]5C)c4cn3)n2)C[C@@H]1O] |
| | 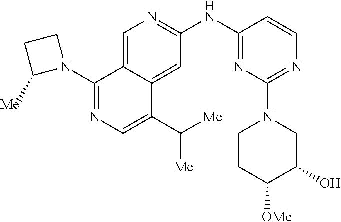 [SMILES: CO[C@@H]1CCN(c2nccc(Nc3cc4c(C(C)C)cnc(N5CC[C@H]5C)c4cn3)n2)C[C@@H]1O] |
| 167 | 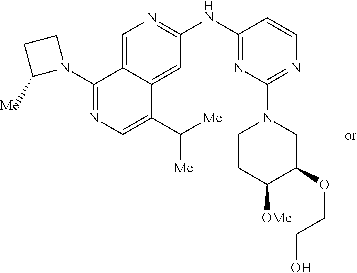 [SMILES: CO[C@H]1CCN(c2nccc(Nc3cc4c(C(C)C)cnc(N5CC[C@H]5C)c4cn3)n2)C[C@H]1OCCO] |
| | 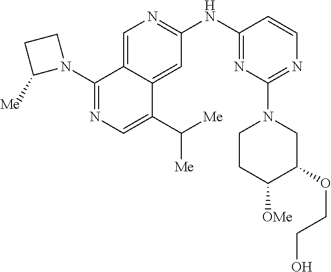 [SMILES: CO[C@@H]1CCN(c2nccc(Nc3cc4c(C(C)C)cnc(N5CC[C@H]5C)c4cn3)n2)C[C@@H]1OCCO] |
| 168 | 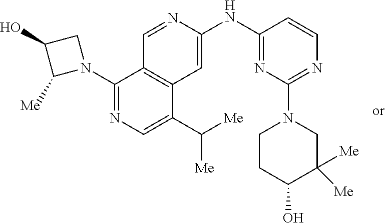 [SMILES: CC(C)c1cnc(N2C[C@H](O)[C@H]2C)c2cnc(Nc3ccnc(N4CC[C@@H](O)C(C)(C)C4)n3)cc12] |
| | 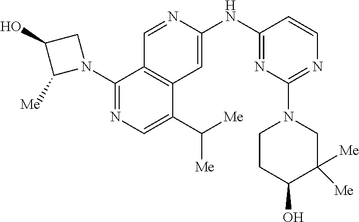 [SMILES: CC(C)c1cnc(N2C[C@H](O)[C@H]2C)c2cnc(Nc3ccnc(N4CC[C@H](O)C(C)(C)C4)n3)cc12] |
| 169 | 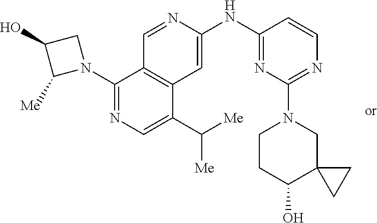 [SMILES: CC(C)c1cnc(N2C[C@H](O)[C@H]2C)c2cnc(Nc3ccnc(N4CC[C@@H](O)C5(CC5)C4)n3)cc12] |
| | 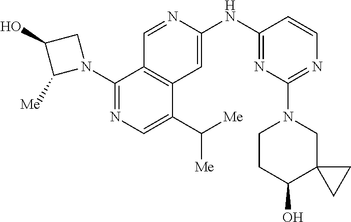 [SMILES: CC(C)c1cnc(N2C[C@H](O)[C@H]2C)c2cnc(Nc3ccnc(N4CC[C@H](O)C5(CC5)C4)n3)cc12] |
| 170 | 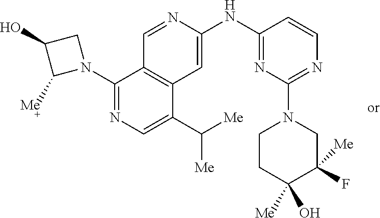 [SMILES: CC(C)c1cnc(N2C[C@H](O)[C@H]2C)c2cnc(Nc3ccnc(N4CC[C@](C)(O)[C@](C)(F)C4)n3)cc12] |
| | 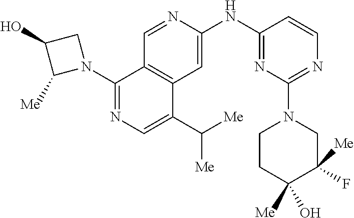 [SMILES: CC(C)c1cnc(N2C[C@H](O)[C@H]2C)c2cnc(Nc3ccnc(N4CC[C@@](C)(O)[C@@](C)(F)C4)n3)cc12] |
| 171 | 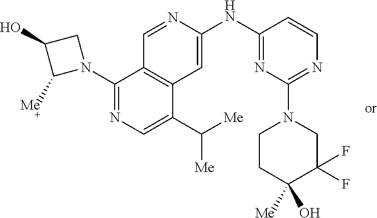 [SMILES: CC(C)c1cnc(N2C[C@H](O)[C@H]2C)c2cnc(Nc3ccnc(N4CC[C@](C)(O)C(F)(F)C4)n3)cc12] |
| | 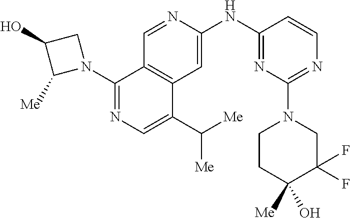 [SMILES: CC(C)c1cnc(N2C[C@H](O)[C@H]2C)c2cnc(Nc3ccnc(N4CC[C@@](C)(O)C(F)(F)C4)n3)cc12] |
| 172 | 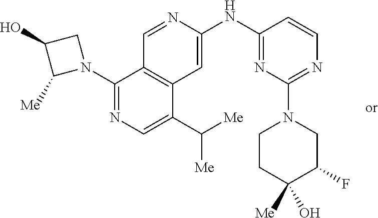 [SMILES: CC(C)c1cnc(N2C[C@H](O)[C@H]2C)c2cnc(Nc3ccnc(N4CC[C@@](C)(O)[C@@H](F)C4)n3)cc12] |
| | 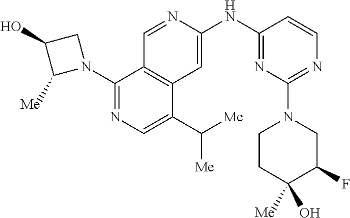 [SMILES: CC(C)c1cnc(N2C[C@H](O)[C@H]2C)c2cnc(Nc3ccnc(N4CC[C@](C)(O)[C@H](F)C4)n3)cc12] |
| 173 | 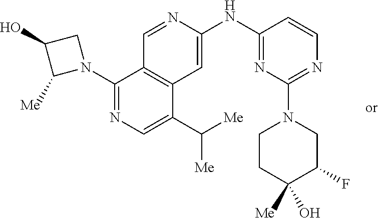 [SMILES: CC(C)c1cnc(N2C[C@H](O)[C@H]2C)c2cnc(Nc3ccnc(N4CC[C@@](C)(O)[C@@H](F)C4)n3)cc12] |
| | 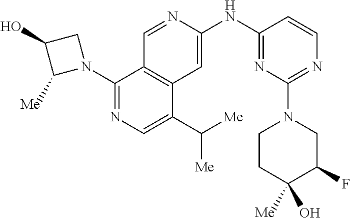 [SMILES: CC(C)c1cnc(N2C[C@H](O)[C@H]2C)c2cnc(Nc3ccnc(N4CC[C@](C)(O)[C@H](F)C4)n3)cc12] |
| 174 | 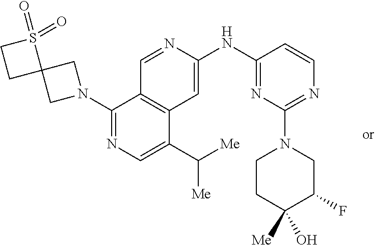 [SMILES: CC(C)c1cnc(N2CC3(CCS3(=O)=O)C2)c2cnc(Nc3ccnc(N4CC[C@@](C)(O)[C@@H](F)C4)n3)cc12] |
| | 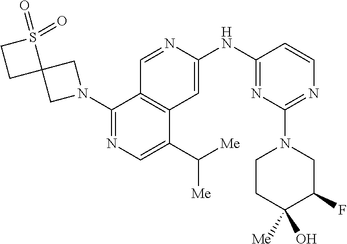 [SMILES: CC(C)c1cnc(N2CC3(CCS3(=O)=O)C2)c2cnc(Nc3ccnc(N4CC[C@](C)(O)[C@H](F)C4)n3)cc12] |
| 175 | 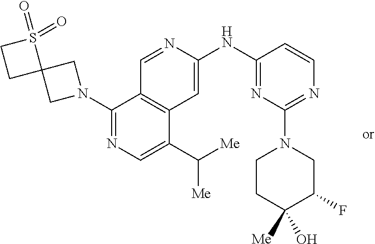 [SMILES: CC(C)c1cnc(N2CC3(CCS3(=O)=O)C2)c2cnc(Nc3ccnc(N4CC[C@@](C)(O)[C@@H](F)C4)n3)cc12] |
| | 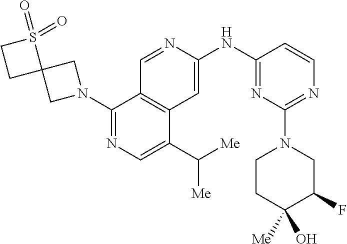 [SMILES: CC(C)c1cnc(N2CC3(CCS3(=O)=O)C2)c2cnc(Nc3ccnc(N4CC[C@](C)(O)[C@H](F)C4)n3)cc12] |
| 176 | 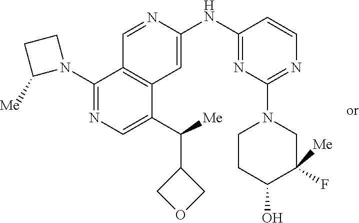 [SMILES: C[C@H](c1cnc(N2CC[C@H]2C)c2cnc(Nc3ccnc(N4CC[C@@H](O)[C@@](C)(F)C4)n3)cc12)C1COC1] |
| | 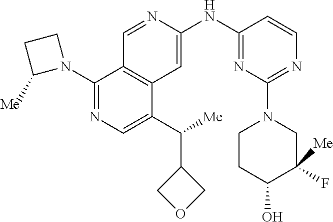 [SMILES: C[C@@H]1CCN1c1ncc([C@H](C)C2COC2)c2cc(Nc3ccnc(N4CC[C@@H](O)[C@@](C)(F)C4)n3)ncc12] |
| 177 | 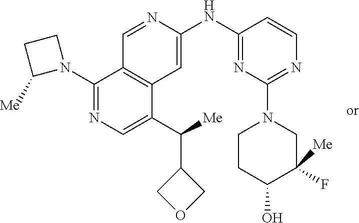 [SMILES: C[C@H](c1cnc(N2CC[C@H]2C)c2cnc(Nc3ccnc(N4CC[C@@H](O)[C@@](C)(F)C4)n3)cc12)C1COC1] |
| | 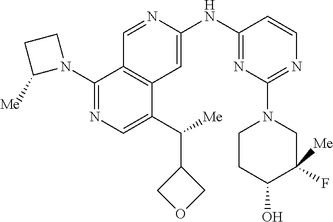 [SMILES: C[C@@H]1CCN1c1ncc([C@H](C)C2COC2)c2cc(Nc3ccnc(N4CC[C@@H](O)[C@@](C)(F)C4)n3)ncc12] |
| 178 | 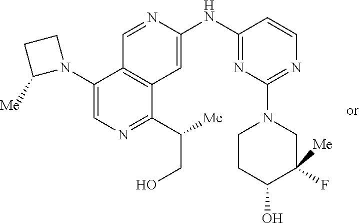 [SMILES: C[C@H](CO)c1ncc(N2CC[C@H]2C)c2cnc(Nc3ccnc(N4CC[C@@H](O)[C@@](C)(F)C4)n3)cc12] |
| | 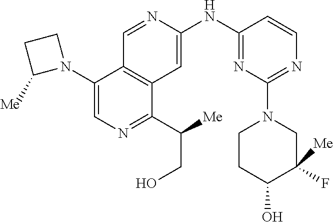 [SMILES: C[C@@H]1CCN1c1cnc([C@@H](C)CO)c2cc(Nc3ccnc(N4CC[C@@H](O)[C@@](C)(F)C4)n3)ncc12] |
| 179 | 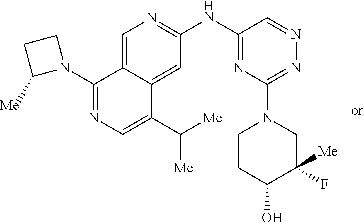 [SMILES: CC(C)c1cnc(N2CC[C@H]2C)c2cnc(Nc3cnnc(N4CC[C@@H](O)[C@@](C)(F)C4)n3)cc12] |
| | 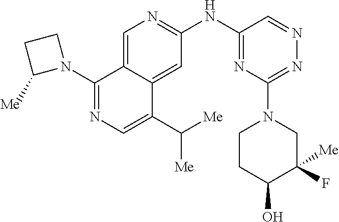 [SMILES: CC(C)c1cnc(N2CC[C@H]2C)c2cnc(Nc3cnnc(N4CC[C@H](O)[C@](C)(F)C4)n3)cc12] |
| 180 | 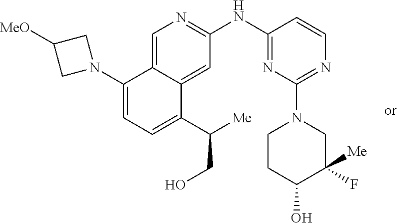 [SMILES: COC1CN(c2ccc([C@@H](C)CO)c3cc(Nc4ccnc(N5CC[C@@H](O)[C@@](C)(F)C5)n4)ncc23)C1] |
| | 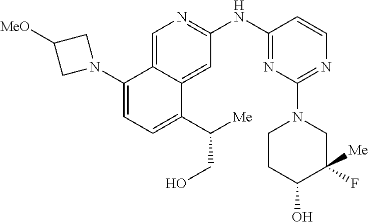 [SMILES: COC1CN(c2ccc([C@H](C)CO)c3cc(Nc4ccnc(N5CC[C@@H](O)[C@@](C)(F)C5)n4)ncc23)C1] |
| 181 | 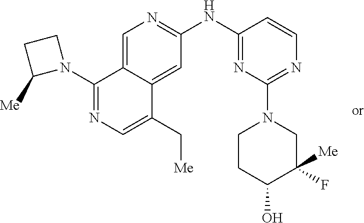 [SMILES: CCc1cnc(N2CC[C@@H]2C)c2cnc(Nc3ccnc(N4CC[C@@H](O)[C@@](C)(F)C4)n3)cc12] |
| | 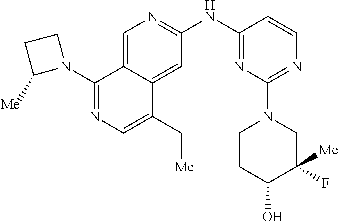 [SMILES: CCc1cnc(N2CC[C@H]2C)c2cnc(Nc3ccnc(N4CC[C@@H](O)[C@@](C)(F)C4)n3)cc12] |
| 182 | 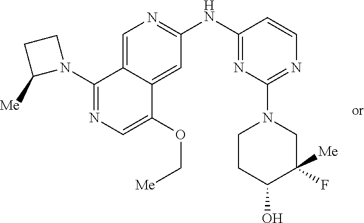 [SMILES: CCOc1cnc(N2CC[C@@H]2C)c2cnc(Nc3ccnc(N4CC[C@@H](O)[C@@](C)(F)C4)n3)cc12] |
| | 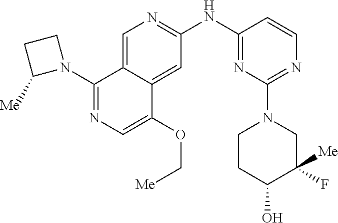 [SMILES: CCOc1cnc(N2CC[C@H]2C)c2cnc(Nc3ccnc(N4CC[C@@H](O)[C@@](C)(F)C4)n3)cc12] |
| 183 | 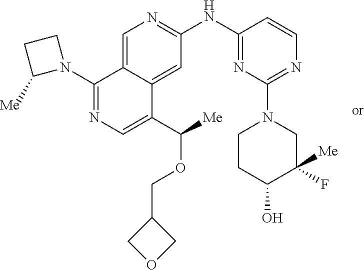 [SMILES: C[C@@H]1CCN1c1ncc([C@@H](C)OCC2COC2)c2cc(Nc3ccnc(N4CC[C@@H](O)[C@@](C)(F)C4)n3)ncc12] |
| | 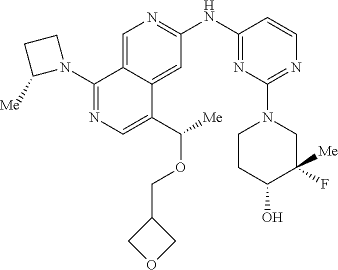 [SMILES: C[C@H](OCC1COC1)c1cnc(N2CC[C@H]2C)c2cnc(Nc3ccnc(N4CC[C@@H](O)[C@@](C)(F)C4)n3)cc12] |
| 184 | 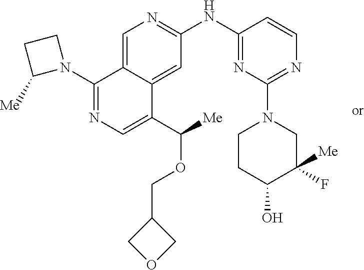 [SMILES: C[C@@H]1CCN1c1ncc([C@@H](C)OCC2COC2)c2cc(Nc3ccnc(N4CC[C@@H](O)[C@@](C)(F)C4)n3)ncc12] |
| | 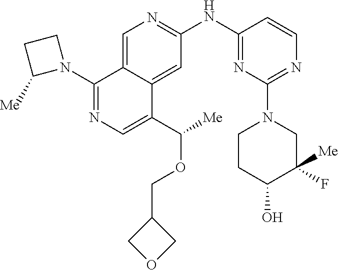 [SMILES: C[C@H](OCC1COC1)c1cnc(N2CC[C@H]2C)c2cnc(Nc3ccnc(N4CC[C@@H](O)[C@@](C)(F)C4)n3)cc12] |
| 185 | 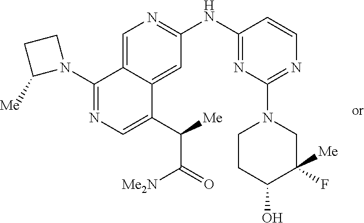 [SMILES: C[C@@H]1CCN1c1ncc([C@@H](C)C(=O)N(C)C)c2cc(Nc3ccnc(N4CC[C@@H](O)[C@@](C)(F)C4)n3)ncc12] |
| | 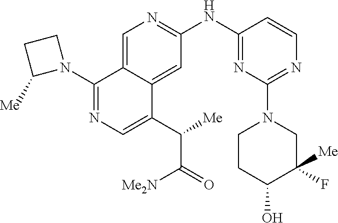 [SMILES: C[C@H](C(=O)N(C)C)c1cnc(N2CC[C@H]2C)c2cnc(Nc3ccnc(N4CC[C@@H](O)[C@@](C)(F)C4)n3)cc12] |
| 186 | 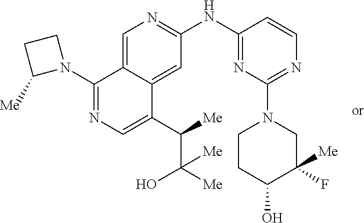 [SMILES: C[C@@H]1CCN1c1ncc([C@@H](C)C(C)(C)O)c2cc(Nc3ccnc(N4CC[C@@H](O)[C@@](C)(F)C4)n3)ncc12] |
| | 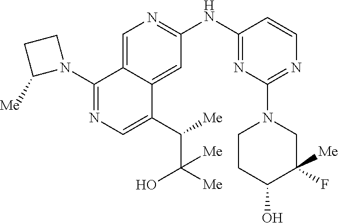 [SMILES: C[C@@H]1CCN1c1ncc([C@H](C)C(C)(C)O)c2cc(Nc3ccnc(N4CC[C@@H](O)[C@@](C)(F)C4)n3)ncc12] |
| 187 | 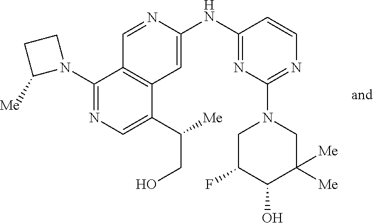 [SMILES: C[C@H](CO)c1cnc(N2CC[C@H]2C)c2cnc(Nc3ccnc(N4C[C@@H](F)[C@@H](O)C(C)(C)C4)n3)cc12] |
| | 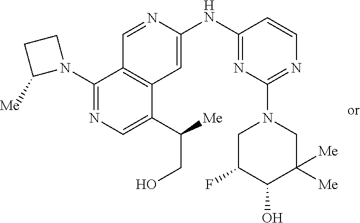 [SMILES: C[C@@H]1CCN1c1ncc([C@@H](C)CO)c2cc(Nc3ccnc(N4C[C@@H](F)[C@@H](O)C(C)(C)C4)n3)ncc12] |
| | 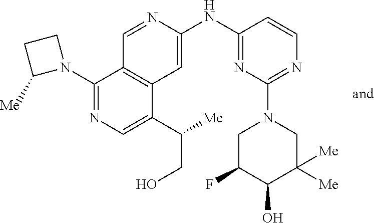 [SMILES: C[C@H](CO)c1cnc(N2CC[C@H]2C)c2cnc(Nc3ccnc(N4C[C@H](F)[C@H](O)C(C)(C)C4)n3)cc12] |
| | 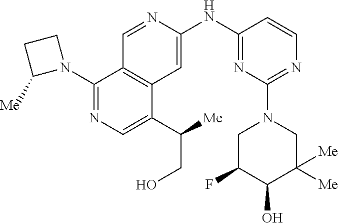 [SMILES: C[C@@H]1CCN1c1ncc([C@@H](C)CO)c2cc(Nc3ccnc(N4C[C@H](F)[C@H](O)C(C)(C)C4)n3)ncc12] |
| 188 | 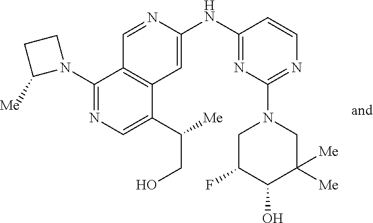 [SMILES: C[C@H](CO)c1cnc(N2CC[C@H]2C)c2cnc(Nc3ccnc(N4C[C@@H](F)[C@@H](O)C(C)(C)C4)n3)cc12] |
| | 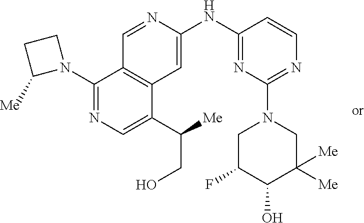 [SMILES: C[C@@H]1CCN1c1ncc([C@@H](C)CO)c2cc(Nc3ccnc(N4C[C@@H](F)[C@@H](O)C(C)(C)C4)n3)ncc12] |
| | 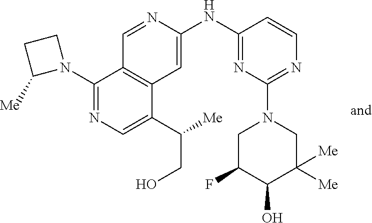 [SMILES: C[C@H](CO)c1cnc(N2CC[C@H]2C)c2cnc(Nc3ccnc(N4C[C@H](F)[C@H](O)C(C)(C)C4)n3)cc12] |
| | 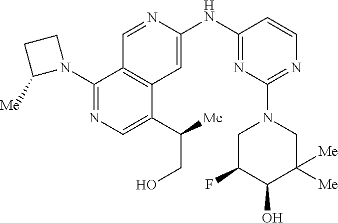 [SMILES: C[C@@H]1CCN1c1ncc([C@@H](C)CO)c2cc(Nc3ccnc(N4C[C@H](F)[C@H](O)C(C)(C)C4)n3)ncc12] |
| 189 | 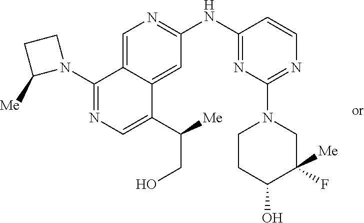 [SMILES: C[C@@H](CO)c1cnc(N2CC[C@@H]2C)c2cnc(Nc3ccnc(N4CC[C@@H](O)[C@@](C)(F)C4)n3)cc12] |
| | 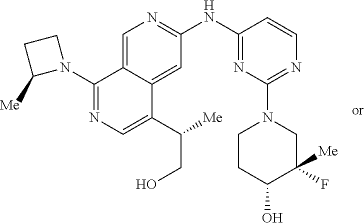 [SMILES: C[C@H](CO)c1cnc(N2CC[C@@H]2C)c2cnc(Nc3ccnc(N4CC[C@@H](O)[C@@](C)(F)C4)n3)cc12] |
| | 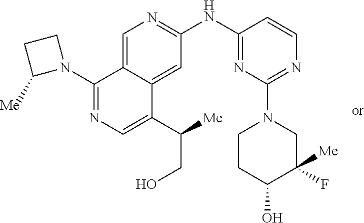 [SMILES: C[C@@H]1CCN1c1ncc([C@@H](C)CO)c2cc(Nc3ccnc(N4CC[C@@H](O)[C@@](C)(F)C4)n3)ncc12] |
| | 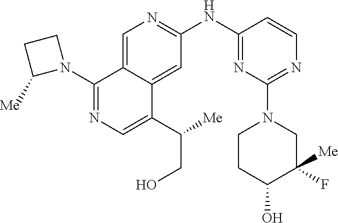 [SMILES: C[C@H](CO)c1cnc(N2CC[C@H]2C)c2cnc(Nc3ccnc(N4CC[C@@H](O)[C@@](C)(F)C4)n3)cc12] |
| 190 | 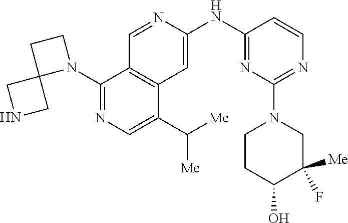 [SMILES: CC(C)c1cnc(N2CCC23CNC3)c2cnc(Nc3ccnc(N4CC[C@@H](O)[C@@](C)(F)C4)n3)cc12] |
| 191 | 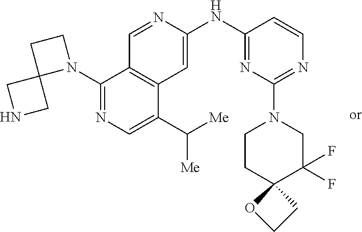 [SMILES: CC(C)c1cnc(N2CCC23CNC3)c2cnc(Nc3ccnc(N4CC[C@@]5(CCO5)C(F)(F)C4)n3)cc12] |
| | 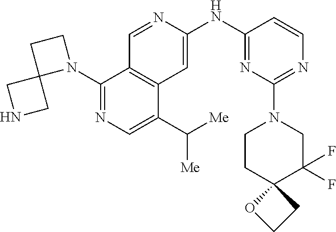 [SMILES: CC(C)c1cnc(N2CCC23CNC3)c2cnc(Nc3ccnc(N4CC[C@]5(CCO5)C(F)(F)C4)n3)cc12] |
| 192 | 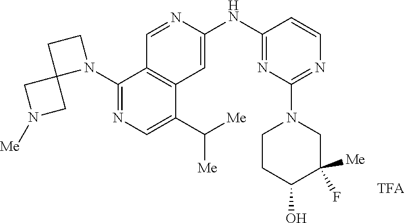 [SMILES: CC(C)c1cnc(N2CCC23CN(C)C3)c2cnc(Nc3ccnc(N4CC[C@@H](O)[C@@](C)(F)C4)n3)cc12] |
| 193 | 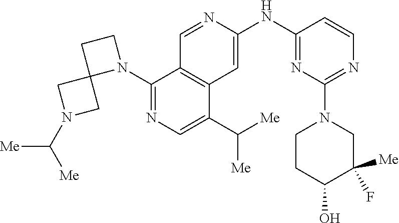 [SMILES: CC(C)c1cnc(N2CCC23CN(C(C)C)C3)c2cnc(Nc3ccnc(N4CC[C@@H](O)[C@@](C)(F)C4)n3)cc12] |
| 194 | 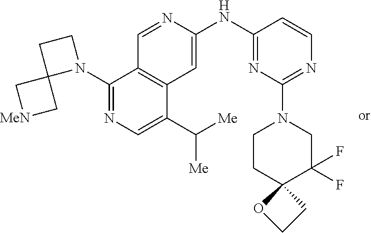 [SMILES: CC(C)c1cnc(N2CCC23CN(C)C3)c2cnc(Nc3ccnc(N4CC[C@@]5(CCO5)C(F)(F)C4)n3)cc12] |
| | 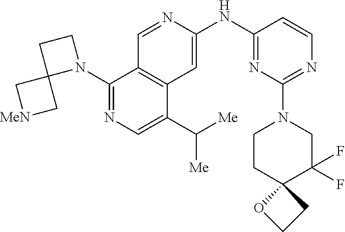 [SMILES: CC(C)c1cnc(N2CCC23CN(C)C3)c2cnc(Nc3ccnc(N4CC[C@]5(CCO5)C(F)(F)C4)n3)cc12] |
| 195 | 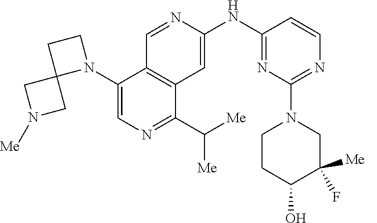 [SMILES: CC(C)c1ncc(N2CCC23CN(C)C3)c2cnc(Nc3ccnc(N4CC[C@@H](O)[C@@](C)(F)C4)n3)cc12] |
| 196 | 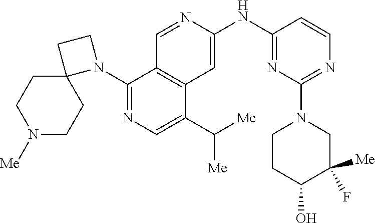 [SMILES: CC(C)c1cnc(N2CCC23CCN(C)CC3)c2cnc(Nc3ccnc(N4CC[C@@H](O)[C@@](C)(F)C4)n3)cc12] |
| 197 | 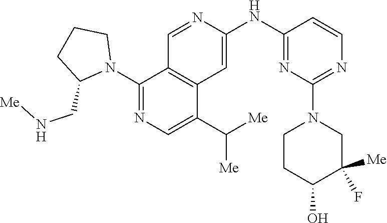 [SMILES: CNC[C@@H]1CCCN1c1ncc(C(C)C)c2cc(Nc3ccnc(N4CC[C@@H](O)[C@@](C)(F)C4)n3)ncc12] |
| 198 | 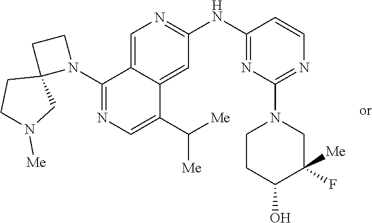 [SMILES: CC(C)c1cnc(N2CC[C@]23CCN(C)C3)c2cnc(Nc3ccnc(N4CC[C@@H](O)[C@@](C)(F)C4)n3)cc12] |
| | 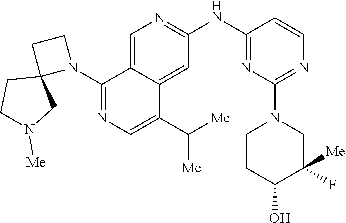 [SMILES: CC(C)c1cnc(N2CC[C@@]23CCN(C)C3)c2cnc(Nc3ccnc(N4CC[C@@H](O)[C@@](C)(F)C4)n3)cc12] |
| 199 | 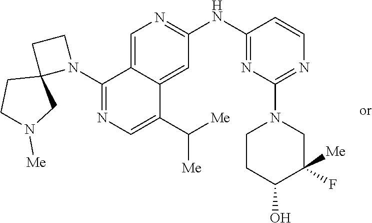 [SMILES: CC(C)c1cnc(N2CC[C@@]23CCN(C)C3)c2cnc(Nc3ccnc(N4CC[C@@H](O)[C@@](C)(F)C4)n3)cc12] |
| | 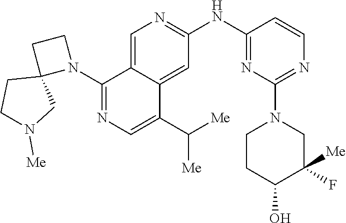 [SMILES: CC(C)c1cnc(N2CC[C@]23CCN(C)C3)c2cnc(Nc3ccnc(N4CC[C@@H](O)[C@@](C)(F)C4)n3)cc12] |
| 200 | 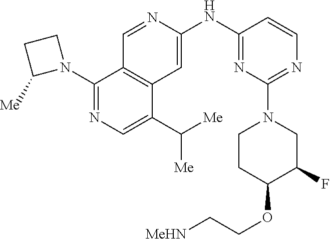 [SMILES: CNCCO[C@H]1CCN(c2nccc(Nc3cc4c(C(C)C)cnc(N5CC[C@H]5C)c4cn3)n2)C[C@H]1F] |
| 201 | 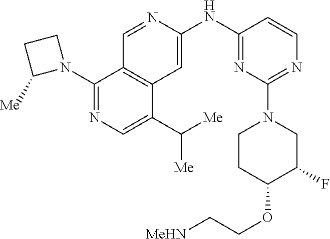 [SMILES: CNCCO[C@@H]1CCN(c2nccc(Nc3cc4c(C(C)C)cnc(N5CC[C@H]5C)c4cn3)n2)C[C@@H]1F] |
| 202 | 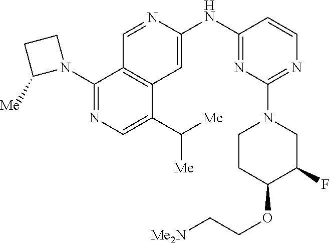 [SMILES: CC(C)c1cnc(N2CC[C@H]2C)c2cnc(Nc3ccnc(N4CC[C@H](OCCN(C)C)[C@H](F)C4)n3)cc12] |
| 203 | 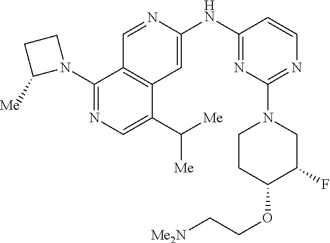 [SMILES: CC(C)c1cnc(N2CC[C@H]2C)c2cnc(Nc3ccnc(N4CC[C@@H](OCCN(C)C)[C@@H](F)C4)n3)cc12] |
| 204 | 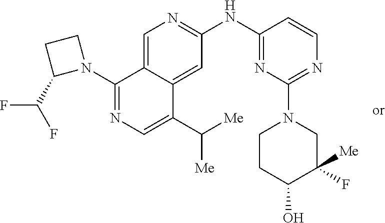 [SMILES: CC(C)c1cnc(N2CC[C@H]2C(F)F)c2cnc(Nc3ccnc(N4CC[C@@H](O)[C@@](C)(F)C4)n3)cc12] |
| | 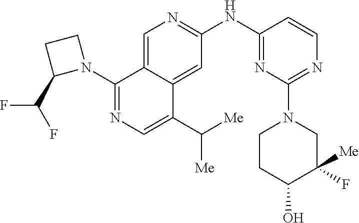 [SMILES: CC(C)c1cnc(N2CC[C@@H]2C(F)F)c2cnc(Nc3ccnc(N4CC[C@@H](O)[C@@](C)(F)C4)n3)cc12] |
| 205 | 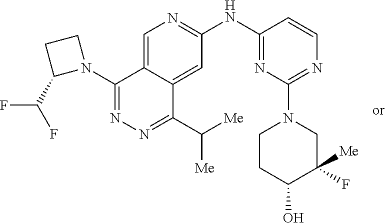 [SMILES: CC(C)c1nnc(N2CC[C@H]2C(F)F)c2cnc(Nc3ccnc(N4CC[C@@H](O)[C@@](C)(F)C4)n3)cc12] |
| | 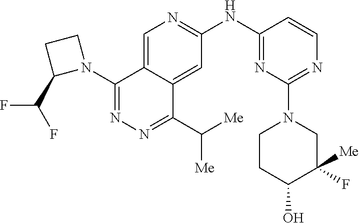 [SMILES: CC(C)c1nnc(N2CC[C@@H]2C(F)F)c2cnc(Nc3ccnc(N4CC[C@@H](O)[C@@](C)(F)C4)n3)cc12] |
| 206 | 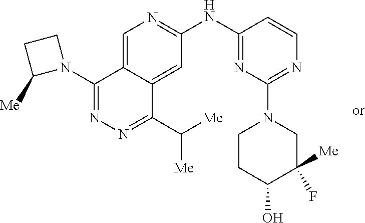 [SMILES: CC(C)c1nnc(N2CC[C@@H]2C)c2cnc(Nc3ccnc(N4CC[C@@H](O)[C@@](C)(F)C4)n3)cc12] |
| | 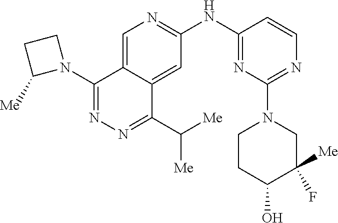 [SMILES: CC(C)c1nnc(N2CC[C@H]2C)c2cnc(Nc3ccnc(N4CC[C@@H](O)[C@@](C)(F)C4)n3)cc12] |
| 207 | 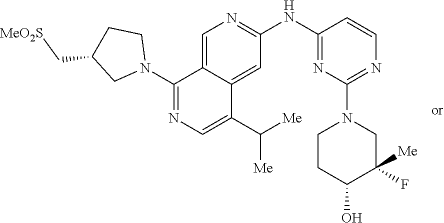 [SMILES: CC(C)c1cnc(N2CC[C@@H](CS(C)(=O)=O)C2)c2cnc(Nc3ccnc(N4CC[C@@H](O)[C@@](C)(F)C4)n3)cc12] |
| | 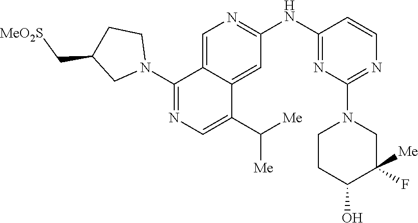 [SMILES: CC(C)c1cnc(N2CC[C@H](CS(C)(=O)=O)C2)c2cnc(Nc3ccnc(N4CC[C@@H](O)[C@@](C)(F)C4)n3)cc12] |
| 208 | 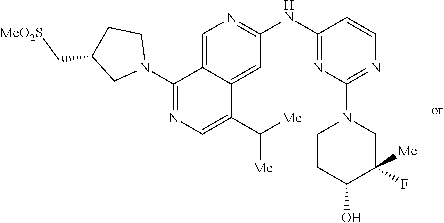 [SMILES: CC(C)c1cnc(N2CC[C@@H](CS(C)(=O)=O)C2)c2cnc(Nc3ccnc(N4CC[C@@H](O)[C@@](C)(F)C4)n3)cc12] |
| | 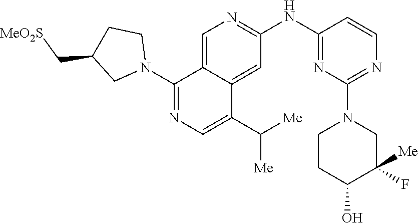 [SMILES: CC(C)c1cnc(N2CC[C@H](CS(C)(=O)=O)C2)c2cnc(Nc3ccnc(N4CC[C@@H](O)[C@@](C)(F)C4)n3)cc12] |
| 209 | 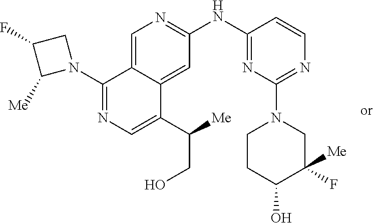 [SMILES: C[C@@H]1[C@H](F)CN1c1ncc([C@@H](C)CO)c2cc(Nc3ccnc(N4CC[C@@H](O)[C@@](C)(F)C4)n3)ncc12] |
| | 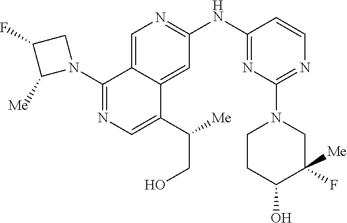 [SMILES: C[C@H](CO)c1cnc(N2C[C@@H](F)[C@H]2C)c2cnc(Nc3ccnc(N4CC[C@@H](O)[C@@](C)(F)C4)n3)cc12] |
| 210 | 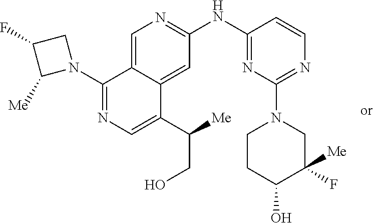 [SMILES: C[C@@H]1[C@H](F)CN1c1ncc([C@@H](C)CO)c2cc(Nc3ccnc(N4CC[C@@H](O)[C@@](C)(F)C4)n3)ncc12] |
| | 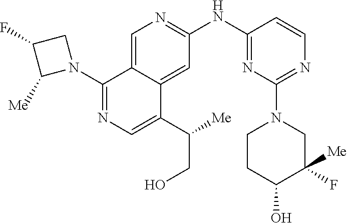 [SMILES: C[C@H](CO)c1cnc(N2C[C@@H](F)[C@H]2C)c2cnc(Nc3ccnc(N4CC[C@@H](O)[C@@](C)(F)C4)n3)cc12] |
| 211 | 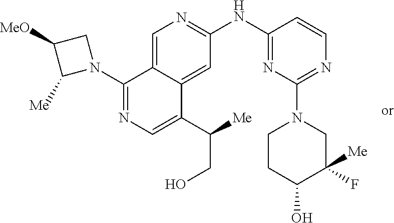 [SMILES: CO[C@H]1CN(c2ncc([C@@H](C)CO)c3cc(Nc4ccnc(N5CC[C@@H](O)[C@@](C)(F)C5)n4)ncc23)[C@@H]1C] |
| | 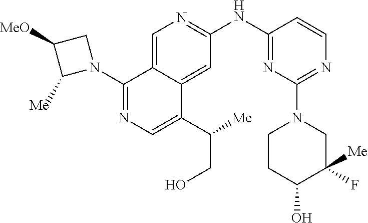 [SMILES: CO[C@H]1CN(c2ncc([C@H](C)CO)c3cc(Nc4ccnc(N5CC[C@@H](O)[C@@](C)(F)C5)n4)ncc23)[C@@H]1C] |
| 212 | 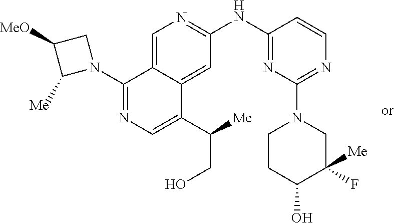 [SMILES: CO[C@H]1CN(c2ncc([C@@H](C)CO)c3cc(Nc4ccnc(N5CC[C@@H](O)[C@@](C)(F)C5)n4)ncc23)[C@@H]1C] |
| | 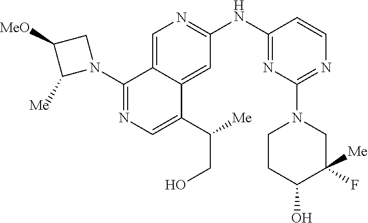 [SMILES: CO[C@H]1CN(c2ncc([C@H](C)CO)c3cc(Nc4ccnc(N5CC[C@@H](O)[C@@](C)(F)C5)n4)ncc23)[C@@H]1C] |
| 213 | 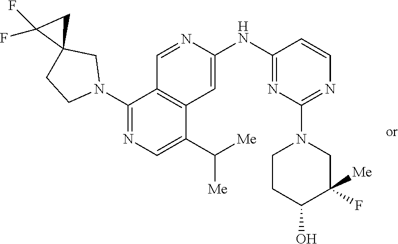 [SMILES: CC(C)c1cnc(N2CC[C@]3(C2)CC3(F)F)c2cnc(Nc3ccnc(N4CC[C@@H](O)[C@@](C)(F)C4)n3)cc12] |
| | 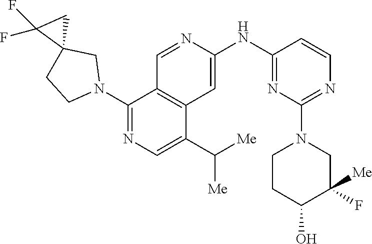 [SMILES: CC(C)c1cnc(N2CC[C@@]3(C2)CC3(F)F)c2cnc(Nc3ccnc(N4CC[C@@H](O)[C@@](C)(F)C4)n3)cc12] |
| 214 | 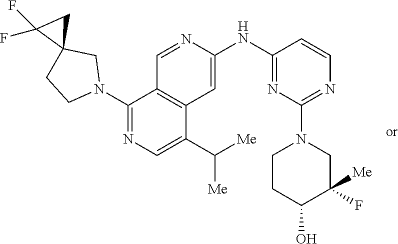 [SMILES: CC(C)c1cnc(N2CC[C@]3(C2)CC3(F)F)c2cnc(Nc3ccnc(N4CC[C@@H](O)[C@@](C)(F)C4)n3)cc12] |
| | 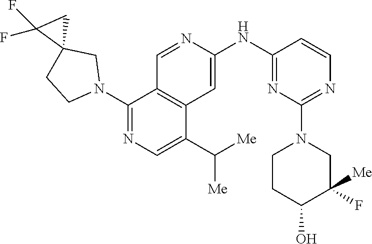 [SMILES: CC(C)c1cnc(N2CC[C@@]3(C2)CC3(F)F)c2cnc(Nc3ccnc(N4CC[C@@H](O)[C@@](C)(F)C4)n3)cc12] |
| 215 | 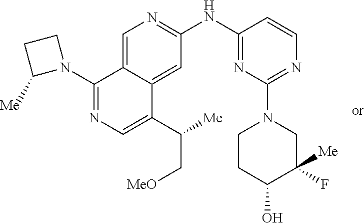 [SMILES: COC[C@@H](C)c1cnc(N2CC[C@H]2C)c2cnc(Nc3ccnc(N4CC[C@@H](O)[C@@](C)(F)C4)n3)cc12] |
| | 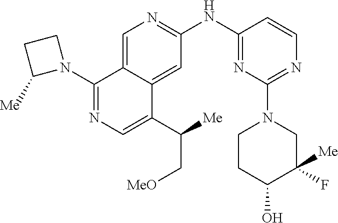 [SMILES: COC[C@H](C)c1cnc(N2CC[C@H]2C)c2cnc(Nc3ccnc(N4CC[C@@H](O)[C@@](C)(F)C4)n3)cc12] |
| 216 | 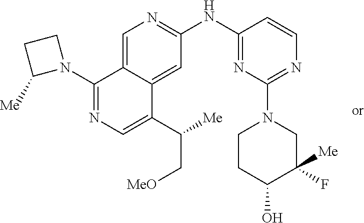 [SMILES: COC[C@@H](C)c1cnc(N2CC[C@H]2C)c2cnc(Nc3ccnc(N4CC[C@@H](O)[C@@](C)(F)C4)n3)cc12] |
| | 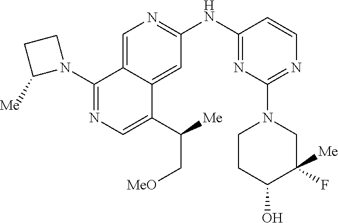 [SMILES: COC[C@H](C)c1cnc(N2CC[C@H]2C)c2cnc(Nc3ccnc(N4CC[C@@H](O)[C@@](C)(F)C4)n3)cc12] |
| 217 | 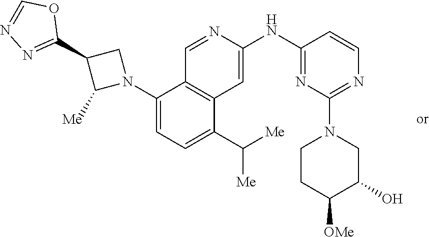 [SMILES: CO[C@H]1CCN(c2nccc(Nc3cc4c(C(C)C)ccc(N5C[C@H](c6nnco6)[C@H]5C)c4cn3)n2)C[C@@H]1O] |
| | 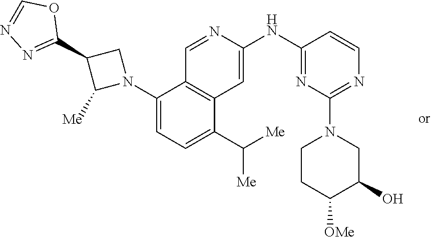 [SMILES: CO[C@@H]1CCN(c2nccc(Nc3cc4c(C(C)C)ccc(N5C[C@H](c6nnco6)[C@H]5C)c4cn3)n2)C[C@H]1O] |
| | 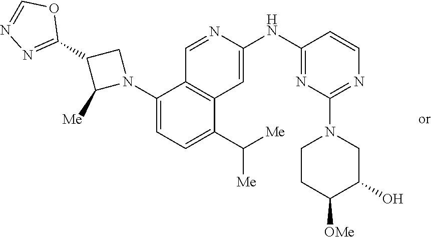 |
| | 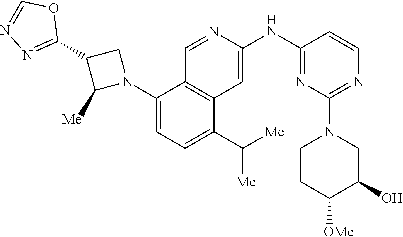 |
| 218 | 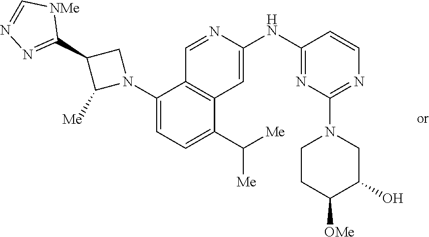 [SMILES: CO[C@H]1CCN(c2nccc(Nc3cc4c(C(C)C)ccc(N5C[C@H](c6nncn6C)[C@H]5C)c4cn3)n2)C[C@@H]1O] |
| | 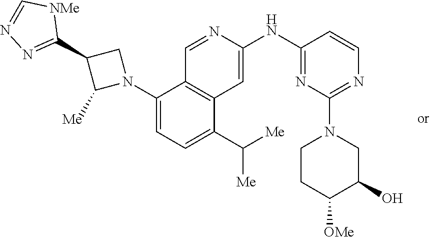 [SMILES: CO[C@@H]1CCN(c2nccc(Nc3cc4c(C(C)C)ccc(N5C[C@H](c6nncn6C)[C@H]5C)c4cn3)n2)C[C@H]1O] |
| | 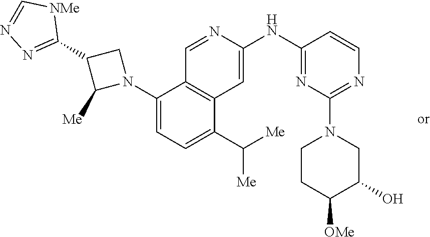 |
| | 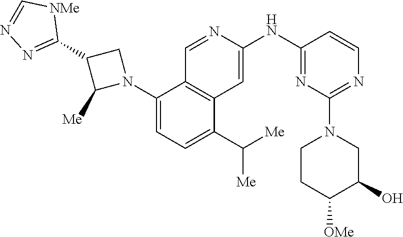 |
| 219 | 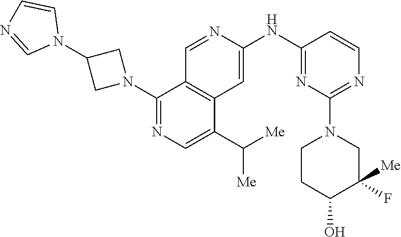 [SMILES: CC(C)c1cnc(N2CC(n3ccnc3)C2)c2cnc(Nc3ccnc(N4CC[C@@H](O)[C@@](C)(F)C4)n3)cc12] |

### Biological Example 1. Biochemical EGFR Inhibition Assays

Inhibitory effects of the compounds of the disclosure were measured in biochemical assays that measure the phosphorylation activity of EGFR enzyme phosphorylates 2.5 micromolar 5-FAM-EEPLYWSFPAKKK-CONH₂ peptide substrate (FL-Peptide 22, PerkinElmer, 760366) in the presence of adenosine-5′-triphosphate (ATP) and varying concentrations of the test compound in 100 mM 2-[4-(2-hydroxyethyl)piperazin-1-yl] ethanesulfonic acid (HEPES), pH 7.5, 10 mM MgCl₂, 0.015% Brij-35, 1 mM dithiothreitol (DTT), 1.0% dimethylsulfoxide (DMSO). Assays were performed at 1.0 mM ATP or at ATP Km of the EGFR enzymes. Reactions proceeded until between 10% to 20% total peptides were phosphorylated at room temperature (25° C.) and were terminated with 35 mM 2,2′,2″,2′″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following enzyme forms of EGFR were examples that were used in these assays:
EGFR WT (SignalChem, E10-112G)
EGFR (L858R T790M C797S) (SignalChem, E10-122VG)
EGFR (d746-750) T790M C797S (SignalChem, E10-122UG)
EGFR L858R (SignalChem, E10-122BG)
EGFR (d746-750) (SignalChem, E10-122JG)

### Biological Example 2. NCI-H1975 pEGFR AlphaLISA Assays

Inhibitory effects of the compounds of the disclosure were evaluated in cellular assays that measure level of intracellular phosphorylation of EGFR in NCI-H1975 cell line that harbors the EGFR L858R T790M mutations (ATCC, CRL-5908) using AlphaLISA sureFire ultra p-EGFR (Tyr1068) assay kit (PerkinElmer, ALSU-PEGFR-A50K). The NCI-H1975 cells were seeded at 12.5K/well in 22 μL into 384 well opti plate (PerkinElmer, 6007299) and adhering overnight at 37C/5% CO₂. On the next day, the test compounds and DMSO control were added into H1975 cell plate followed by incubation at 37C/5% CO₂ for 4-5 hours. The cells were then spin down in the 384-well plate and lysed with 10 μL of 1× AlphaLISA lysis buffer followed by shaking at 600 rpm for 10 minutes at room temperature. After that, 5 μL of an acceptor bead mix was added to each well followed by incubation at room temperature for 1.5-2 h in dark. Then 5 μL of a donor bead mix was added to each well followed by overnight incubation at room temperature in dark. On the next day, the plate was read at a compatible plate reader to obtain pEGFR signal. Percent of pEGFR inhibition was plotted against log concentration of compounds to generate IC₅₀ values. Biological assay data of the test compounds are provided in Table 2 below. For inhibitory activity against EGFR LRTMCS mutant, the following designations are used: ≤15 nM=A; >15-20 nM=B; >20-30 nM=C; >30-100 nM=D and >100=E. For inhibition of phosphorylation of mutant EGFR in cells: ≤10 nM=A; >10-20 nM=B; >20-30 nM=C; >30-50 nM=D; and >50 nM=E.

TABLE 2
Tabularized Data:

| | | Ext pEGFR |
|---|---|---|
| | LRTMCS | H1975 |
| | (PH) | GMean |
| | IC50 | IC50 (nM) |
| | (nM) | Protocol: |
| | @ 1 mM | @Pharmaron |
| | ATP | AlphaLISA |
| Example | (Num) | (Num) |
| 1 | A | A |
| 2 | A | E |
| 3 | A | C |
| 4 | A | C |
| 5 | A | B |
| 6 | A | E |
| 7 | B | E |
| 8 | C | E |
| 9 | A | D |
| 10 | A | B |
| 11 | C | D |
| 12 | A | B |
| 13 | D | E |
| 14 | A | E |
| 15 | A | E |
| 16 | B | E |
| 17 | A | B |
| 18 | B | D |
| 20 | A | D |
| 21 | A | E |
| 22 | B | E |
| 23 | A | E |
| 24 | A | D |
| 25 | B | D |
| 26 | A | D |
| 27 | A | E |
| 28 | A | A |
| 29 | A | A |
| 30 | A | A |
| 31 | A | C |
| 32 | A | A |
| 33 | A | A |
| 34 | A | E |
| 35 | A | A |
| 36 | A | A |
| 37 | A | B |
| 38 | A | A |
| 39 | A | E |
| 40 | A | C |
| 41 | A | D |
| 42 | C | E |
| 43 | B | E |
| 44 | A | C |
| 45 | C | E |
| 46 | A | C |
| 47 | A | C |
| 48 | A | B |
| 49 | B | D |
| 50 | C | E |
| 51 | A | C |
| 52 | A | A |
| 53 | A | C |
| 54 | B | C |
| 55 | A | B |
| 56 | A | E |
| 57 | A | C |
| 58 | A | B |
| 59 | A | B |
| 60 | A | B |
| 61 | A | D |
| 62 | A | D |
| 63 | B | C |
| 64 | A | D |
| 65 | A | C |
| 66 | C | D |
| 67 | B | E |
| 68 | C | D |
| 69 | A | D |
| 70 | B | E |
| 71 | C | D |
| 72 | B | E |
| 73 | D | E |
| 74 | A | A |
| 75 | B | B |
| 76 | A | B |
| 77 | B | C |
| 78 | B | C |
| 79 | D | E |
| 80 | A | D |
| 81 | B | E |
| 82 | B | D |
| 83 | A | C |
| 84 | A | C |
| 85 | C | D |
| 86 | C | D |
| 87 | A | C |
| 88 | A | D |
| 89 | A | A |
| 90 | A | D |
| 91 | B | E |
| 92 | B | D |
| 93 | A | B |
| 94 | A | C |
| 95 | A | B |
| 96 | C | C |
| 97 | A | B |
| 98 | A | B |
| 99 | D | D |
| 100 | A | B |
| 101 | A | D |
| 102 | C | E |
| 103 | A | B |
| 104 | A | A |
| 105 | A | C |
| 106 | A | C |
| 107 | A | A |
| 108 | A | B |
| 109 | C | B |
| 110 | D | E |
| 111 | A | A |
| 112 | A | B |
| 113 | C | B |
| 114 | D | E |
| 115 | D | D |
| 116 | C | E |
| 117 | B | E |
| 118 | C | E |
| 119 | C | E |
| 120 | C | D |
| 121 | D | E |
| 122 | A | B |
| 123 | B | E |
| 124 | A | D |
| 125 | A | D |
| 126 | A | B |
| 127 | B | B |
| 128 | A | E |
| 129 | D | C |
| 130 | B | C |
| 131 | B | E |
| 132 | A | B |
| 133 | A | B |
| 133 | A | B |
| 134 | D | E |
| 135 | C | E |
| 136 | A | C |
| 137 | A | A |
| 138 | A | B |
| 139 | A | A |
| 140 | A | A |
| 141 | A | A |
| 142 | A | A |
| 143 | A | B |
| 144 | B | D |
| 145 | A | C |
| 146 | A | D |
| 147 | A | C |
| 148 | A | A |
| 149 | A | B |
| 150 | A | A |
| 151 | C | D |
| 152 | B | D |
| 153 | D | E |
| 154 | A | B |
| 155 | A | A |
| 156 | C | E |
| 157 | A | E |
| 158 | B | D |
| 159 | A | B |
| 160 | A | B |
| 161 | A | D |
| 162 | A | D |
| 163 | A | C |
| 164 | A | D |
| 165 | A | C |
| 166 | A | D |
| 167 | A | A |
| 168 | A | D |
| 169 | A | D |
| 170 | A | C |
| 171 | A | D |
| 172 | B | E |
| 173 | A | D |
| 174 | C | E |
| 175 | A | E |
| 176 | A | C |
| 177 | A | C |
| 178 | C | E |
| 179 | A | B |
| 180 | D | E |
| 181 | B | D |
| 182 | D | E |
| 183 | D | E |
| 184 | C | D |
| 185 | C | E |
| 186 | A | B |
| 187 | D | E |
| 188 | A | C |
| 189 | A | B |
| 190 | A | E |
| 191 | A | E |
| 192 | A | D |
| 193 | A | C |
| 194 | A | E |
| 195 | C | D |
| 196 | A | B |
| 197 | A | D |
| 198 | B | D |
| 199 | A | C |
| 200 | A | A |
| 201 | A | B |
| 202 | D | D |
| 203 | A | D |
| 204 | A | B |
| 205 | D | E |
| 206 | A | C |
| 207 | A | C |
| 208 | A | B |
| 209 | B | E |
| 210 | A | B |
| 211 | D | E |
| 212 | A | B |
| 213 | A | B |
| 214 | D | D |
| 215 | A | D |
| 216 | B | C |
| 217 | B | A |
| 218 | A | D |
| 219 | C | E |

Additional compounds falling within the scope of formula (I) not disclosed herein were also tested in the assays described in Biological Examples 1 and 2, and all but three had inhibitory activities of less than 10 micromolar in these assays. The following three compounds had inhibitory activity greater than 10 micromolar in Biological Assays 1 or 2.

TABLE 3

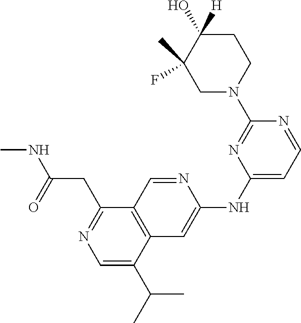 [SMILES: CNC(=O)Cc1ncc(C(C)C)c2cc(Nc3ccnc(N4CC[C@@H](O)[C@@](C)(F)C4)n3)ncc12]

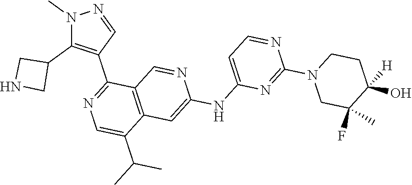 [SMILES: CC(C)c1cnc(-c2cnn(C)c2C2CNC2)c2cnc(Nc3ccnc(N4CC[C@@H](O)[C@@](C)(F)C4)n3)cc12]

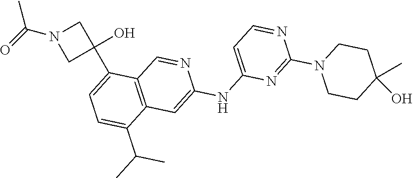 [SMILES: CC(=O)N1CC(O)(c2ccc(C(C)C)c3cc(Nc4ccnc(N5CCC(C)(O)CC5)n4)ncc23)C1]

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize or be able to ascertain using no more than routine experimentation, many equivalents to the specific embodiments of the invention described herein. Such equivalents are intended to be encompassed by the following claims.

1. A compound of Formula (I) 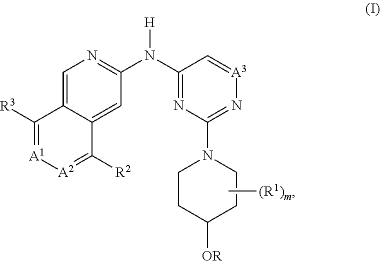
or a pharmaceutically acceptable salt thereof, wherein:
each A¹, A², and A³ is independently N or CR; wherein each R is independently H, halogen, or CH₃;
each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ or in the group represented by R¹ are optionally substituted with 1 to 3 groups selected from deuterium, halogen, OH, NR^{a}R^{b}, C₁-C₂ alkyl, and C₁-C₂ alkoxy; or two R¹, attached to the same carbon atom, taken together with carbon atom to which they are both attached form a C₃-C₄cycloalkyl optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is H, halogen, C₁-C₆ alkyl, C₁-C₆ alkoxy, C₃-C₆ cycloalkyl, 4 to 8 membered heterocyclyl, or 5 to 12-membered heteroaryl, wherein the alkyl, alkoxy, cycloalkyl, or heterocyclyl represented by R² are optionally substituted with 1 to 3 groups selected from R^{2a};
Each R^{2a} is independently selected from halogen, CN, OH, C(O)NR^{a}R^{b}, C₁-C₄ alkyl, C₁-C₄ alkoxy, and 4 to 8 membered heterocyclyl, wherein the alkoxy represented by R^{2a} is optionally substituted with 4 to 8 membered heterocyclyl, and the heterocyclyl represented by R^{2a} or in the group represented by R^{2a} is optionally substituted with C₁-C₄alkyl;
R³ is C₁-C₄ alkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is C₃-C₆cycloalkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O; or
R³ is 5 to 12 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and =0;
R³ is a 4 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O; or
R³ is 5 to 12-membered heteroaryl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O;
R⁴ is H, or C₁-C₄alkyl optionally substituted with 1 to 3 groups selected from deuterium, OR^{a}, and NR^{a}R^{b}, or R⁴, together with R¹ attached to the same carbon atom and their intervening atoms, form a 3 to 5 membered heterocyclyl;
each R^{a} and R^{b} is independently H or C₁-C₄ alkyl optionally substituted with 1 to 3 groups selected from deuterium, halogen, OH and NH₂; and
each R^{c} is independently C₁-C₄ alkyl optionally substituted with 1 to 3 halogen.

2. The compound of claim 1 or a pharmaceutically acceptable salt thereof, wherein A³ is CH.

3. The compound of any one of claim 1 or 2 or a pharmaceutically acceptable salt thereof, wherein each R¹ is independently halogen, CN, OH, NR^{a}R^{b}, C₁-C₄ alkyl, or C₁-C₄ alkoxy, wherein the alkyl or alkoxy represented by R¹ are optionally substituted with 1 to 3 groups selected from deuterium, halogen, OH; or two R¹, attached to the same carbon atom, together with the carbon atoms to which they are both attached form a C₃-C₄cycloalkyl; and/or
m is 0, 1, 2, 3, or 4.

4. The compound of any one of claim 1 to 3 or a pharmaceutically acceptable salt thereof, wherein each R¹ is independently halogen, OH, C₁-C₄ alkyl, or C1-C₄ alkoxy, wherein the alkyl or alkoxy represented by R¹ are optionally substituted with 1 to 3 groups selected from OH; or two R¹, attached to the same carbon atom, together with the carbon atoms to which they are both attached form a C₃-C₄cycloalkyl; and/or
m is 0, 1, 2, or 3.

5. The compound of any one of claim 1 to 4 or a pharmaceutically acceptable salt thereof, wherein R² is C₁-C₆ alkyl, C₁-C₆ alkoxy, C₃-C₆ cycloalkyl, 4 to 6 membered heterocyclyl, or 5 to 6-membered heteroaryl, wherein the alkyl, alkoxy, cycloalkyl, or heterocyclyl represented by R² are optionally substituted with 1 to 3 groups selected from R^{2a};
each R^{2a} is independently selected from halogen, CN, OH, C(O)NR^{a}R^{b}, C₁-C₄ alkyl, C₁-C₄ alkoxy, and 4 to 6 membered heterocyclyl, wherein the alkoxy represented by R^{2a} is optionally substituted with 4 to 6 membered heterocyclyl, and the heterocyclyl represented by R^{2a} or in the group represented by R^{2a} is optionally substituted with C₁-C₄alkyl.

6. The compound of any one of claim 1 to 5 or a pharmaceutically acceptable salt thereof, wherein R² is C₁-C₆ alkyl, C₁-C₆ alkoxy, C₃-C₆ cycloalkyl, 4 to 6 membered heterocyclyl, or 5 to 6-membered heteroaryl, wherein the alkyl, alkoxy, cycloalkyl, or heterocyclyl represented by R² or in the group represented by R² is optionally substituted with 1 to 3 groups selected from R^{2a};
Each R^{2a} is independently selected from halogen, CN, OH, C(O)NR^{a}R^{b}, C₁-C₄ alkoxy, and 4 to 6 membered heterocyclyl, wherein the alkoxy represented by R^{2a} or in the group represented by R^{2a} is optionally substituted with 4 to 6 membered heterocyclyl, and the heterocyclyl represented by R^{2a} or in the group represented by R^{2a} is optionally substituted with C₁-C₄alkyl.

7. The compound of any one of claim 1 to 6 or a pharmaceutically acceptable salt thereof, wherein R³ is C₁-C₄ alkyl optionally substituted with 1 to 3 groups selected from halogen, OR^{a}, ═O, CN, C(O)R^{c}, C(O)OR^{a}, 4 to 6-membered heterocycyl optionally substituted with 1 to 3 groups selected from halogen, deuterium, OR^{a}, CN, ═O, and C(O)R^{c}.

8. The compound of any one of claim 1 to 7 or a pharmaceutically acceptable salt thereof, wherein R³ is C₁-C₄ alkyl optionally substituted with 1 to 3 groups selected from 4 to 6-membered heterocycyl optionally substituted with C(O)R^{c}.

9. The compound of any one of claims 1 to 6 or a pharmaceutically acceptable salt thereof, R³ is C₃-C₆cycloalkyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, and ═O.

10. The compound of any one of claims 1 to 6 and 9 or a pharmaceutically acceptable salt thereof, R³ is C₃-C₆cycloalkyl optionally substituted with 1 to 3 groups selected from S(O)₂CH₃ and C₁-C₄ alkyl optionally substituted with 1 S(O)₂R^{c}.

11. The compound of any one of claims 1 to 6 or a pharmaceutically acceptable salt thereof, R³ is 5 to 10 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

12. The compound of any one of claims 1 to 6 and 11 or a pharmaceutically acceptable salt thereof, R³ is 5 to 10 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

13. The compound of any one of claims 1 to 6 and 11 or a pharmaceutically acceptable salt thereof, R³ is pyrrolidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

14. The compound of any one of claims 1 to 6 and 11 or a pharmaceutically acceptable salt thereof, R³ is 1,4,5,6-tetrahydropyrrolo[3,4-c]pyrazolyl, 1,6-diazaspiro[3.3]heptanyl, 1,6-diazaspiro[3.4]octanyl, 1,7-diazaspiro[3.5]nonanyl, 1-oxa-5-azaspiro[3.3]heptanyl, 1-thia-6-azaspiro[3.3]heptanyl, 2,6-diazaspiro[3.4]octanyl, 5-azaspiro[2.3]hexanyl, 5-azaspiro[2.4]heptanyl, 6-oxa-1-azaspiro[3.3]heptanyl or 5-oxa-2,7-diazaspiro[3.4]octan-6-onyl, each of which are optionally substituted with 1 to 3 groups selected from halogen, ═O, C(O)OR^{a}, and C₁-C₄ alkyl optionally substituted OR^{a}.

15. The compound of any one of claims 1 to 6 or a pharmaceutically acceptable salt thereof, R³ is a 4 membered heterocyclyl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O.

16. The compound of any one of claims 1 to 6 or a pharmaceutically acceptable salt thereof, R³ is a azetidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, OR^{a}, CN, C(O)NR^{a}R^{b}, NR^{a}R^{b}, NR^{a}C(O)OR^{c}, NR^{a}S(O)₂R^{c}, NS(O)(R^{c})₂, P(O)(OR^{c})₂, P(O)(R^{c})₂, S(O)R^{c}, 5-6 membered heteroaryl, C₁-C₄alkyl, and ═O.

17. The compound of any one of claims 1 to 6 or a pharmaceutically acceptable salt thereof, R³ is a azetidinyl optionally substituted with 1 to 3 groups selected from halogen, OR^{a}, ═O, CN, C(O)R^{c}, C(O)OR^{a}, C(O)NR^{a}R^{b}, NR^{a}R^{b}, S(O)₂CH₃, C₁-C₄ alkyl, triazolyl, diazolyl, oxadiazolyl, oxetanyl, and pyrrolidinonyl, wherein the triazolyl, diazolyl, oxadiazolyl, oxetanyl, and pyrrolidinonyl or in the group represented by R³ are optionally substituted with 1 to 3 C₁-C₄alkyl.

18. The compound of any one of claims 1 to 6 or a pharmaceutically acceptable salt thereof, R³ is 5 or 6-membered heteroaryl optionally substituted with 1 to 3 groups selected from halogen, OR^{a}, ═O, CN, C(O)R^{c}, C(O)OR^{a}, C(O)NR^{a}R^{b}, NR^{a}R^{b}, NHC(O)CH₃, S(O)₂CH₃, C₁-C₄ alkyl, 4 to 6-membered heterocycyl, and 5 to 6 membered heteroaryl, wherein the alkyl, heterocycyl and the heteroaryl in the group represented by R³ are optionally substituted with 1 to 3 groups selected from halogen, deuterium, OR^{a}, CN, C(O)R^{c}, C(O)NR^{a}R^{b}, NR^{a}R^{b}, NR^{a}C(O)R^{c}, NR^{a}C(O)OR^{c}, NR^{a}S(O)₂R^{c}, NS(O)(R^{c})₂, P(O)(OR^{c})₂, P(O)(R^{c})₂, S(O)R^{c}, S(O)₂R^{c}, 5-6 membered heteroaryl, C₁-C₄alkyl, and ═O.

19. The compound of any one of claims 1 to 6 or a pharmaceutically acceptable salt thereof, R³ is oxadiazolyl, pyrazolyl or triazolyl, each of which is optionally substituted with 1 to 3 groups selected from halogen and C₁-C₄ alkyl optionally substituted with halogen, OR^{a}, or NR^{a}R^{b}.

20. The compound of any one of claims 1 to 19 or a pharmaceutically acceptable salt thereof, wherein R⁴ is H, or C1-C₄alkyl optionally substituted with 1 to 3 groups selected from deuterium, OR^{a}, and NR^{a}R^{b}, or R⁴, together with R¹ form an oxetanyl.

21. The compound of any one of claims 1 to 20 or a pharmaceutically acceptable salt thereof, each R^{a} and R^{b} is independently H or C₁-C₂ alkyl optionally substituted with 1 to 3 groups selected from deuterium, halogen, OH and NH₂; and each R^{c} is independently C₁-C₂ alkyl optionally substituted with 1 to 3 halogen.

22. The compound of any one of claims 1-21, wherein the compound is a compound of Formula (II) 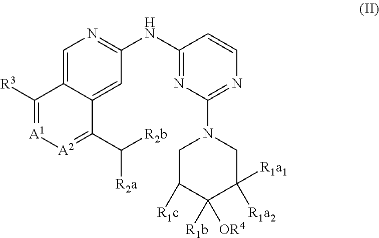
or a pharmaceutically acceptable salt thereof, wherein
R₁ₐ₁ is H, halogen, or C1-C₄alkyl optionally substituted with OH;
R₁ₐ₂ is H, halogen, OH, or C₁-C₄alkyl; or
R₁ₐ₁ and R₁ₐ₂ together with the carbon atom to which they are both attached form a C3-C₄ cycloalkyl;
R₂ₐ is H, CN, oxetane, or C₁-C₃alkyl optionally substituted with CN, OH or methoxy;
R_{2b} is H or methyl; or
R₂ₐ and R_{2b} together with the carbon atom to which they are both attached form a 3-4-member cycloalkyl or 4-6-member heterocyclyl, wherein the cycloalkyl or heterocyclyl is optionally substituted with C₁-C₄alkyl;
R_{1b} is H or C₁-C₄alkyl optionally substituted with OH;
R_{1c} is H or halogen; and
R⁴ is H or C1-C₄alkyl optionally substituted with 1 to 3 deuterium, OH or NR^{a}R^{b}; or
R_{1b} and R₄ together form a 3 to 5-member heterocyclyl ring.

23. The compound of claim 22 or a pharmaceutically acceptable salt thereof, wherein R₂ₐ and R_{2b} are each methyl.

24. The compound of claim 22 or a pharmaceutically acceptable salt thereof, wherein
R₁ₐ₁ is H or methyl and R₁ₐ₂ is F;
R₂ₐ is methyl or methylene substituted with CN;
R_{2b} is methyl;
R_{1b} and R₄ are each independently H or methyl; or R_{1b} and R₄ together form an oxetane ring; and
R_{1c} is H or F.

25. The compound of any one of claims 22 to 24 or a pharmaceutically acceptable salt thereof, wherein
a. R₁ₐ₁ is methyl, R₁ₐ₂ is F, R_{1b} is H and R₄ is H or CH₃;
b. R₁ₐ₁ is H, R₁ₐ₂ is F and R_{1b} is CH₃;
c. R_{1b} and R₄ together with the carbon atom to which they are both attached form an oxetane ring;
d. R₁ₐ₁ is H, R₄ is H and R_{1b} is CH₃; or
e. R⁴ is CH₃, and R₁ₐ₁ is H.

26. The compound of any one of claims 22 to 24 or a pharmaceutically acceptable salt thereof, wherein
a. A² is CH,
b. R₁ₐ₁ is methyl,
c. R₁ₐ₂ is F,
d. R_{1b} is H,
e. R_{1c} is hydrogen,
f. R₂ₐ and R_{2b} are both methyl, and
g. R⁴ is H.

27. A pharmaceutical composition comprising a pharmaceutically acceptable carrier and a compound of any one of claims 1-26, or a pharmaceutically acceptable salt thereof.

28. A method of treating a cancer, comprising administering a subject in need thereof an effective amount of a compound of any of claims 1-26, or a pharmaceutically acceptable salt thereof, or a pharmaceutical composition of claim 27.

29. The method of claim 28, wherein the cancer is non-small cell lung cancer.

30. The method of claim 28 or 29, wherein the cancer in the subject in need thereof has metastasized.

31. The method of any one of claims 28-30, wherein the cancer is characterized by: i) epidermal growth factor receptor EGFR L858R mutation and/or exon 19 deletion; and ii) T790M mutation.

32. The method of claim 31, wherein the cancer is further characterized by epidermal growth factor receptor (EGFR) C797S mutation.

33. The method of any one of claims 28-32, further comprises administering the subject in need thereof an effective amount of afatinib, osimertinib, erlotinib, or gefitinib.

34. A method of inhibiting epidermal growth factor receptor (EGFR), comprising administering to a subject in need thereof an effective amount of a compound of any of claims 1-26, or a pharmaceutically acceptable salt thereof, or a pharmaceutical composition of claim 27.
